(19) United States
(12) Patent Application Publication
(10) Pub. No.: US 2024/0261274 A1
(43) Pub. Date: Aug. 8, 2024

(54) TREATMENT OF MYC-DRIVEN CANCERS WITH GSPT1 DEGRADERS

(71) Applicant: MONTE ROSA THERAPEUTICS, INC., Boston, MA (US)

(72) Inventors: Gerald Gavory, Brinckheim (FR); Mahmoud GHANDI, Basel (CH); Agustin Chicas, Boston, MA (US); Marcus Warmuth, Basel (CH)

(21) Appl. No.: 18/271,954

(22) PCT Filed: Jan. 13, 2022
(86) PCT No.: PCT/EP2022/050702

§ 371 Date: Jul. 12, 2023

(30) Foreign Application Priority Data
Jan. 13, 2021 (CH) 00024/21
Jan. 13, 2021 (CH) 00026/21
Apr. 14, 2021 (CH) 00387/21
Apr. 14, 2021 (CH) 00388/21
Jun. 4, 2021 (CH) 00656/21
Jun. 4, 2021 (CH) 00657/21

(51) Int. Cl. A61K 31/454; A61P 35/00; C12Q 1/6886; G01N 33/574

(52) U.S. Cl. CPC A61K 31/454; A61P 35/00; C12Q 1/6886; G01N 33/57484; C12Q 2600/106; C12Q 2600/158; G01N 2440/14

(57) ABSTRACT

The present disclosure relates to new methods to predict the responsiveness of cancer patients to GSPT1 negative modulators and thus determine the of efficacy GSPT1 negative modulators to treat cancer patients by determining the level of one or more biomarkers in samples of the patients. The present disclosure also relates to applications of these methods, which includes stratifying cancer malignancies, in particular identifying myc-driven cancers, and thereby devising optimized and personalized treatments for these cancer patients, as well as optimizing the selection of patient populations for respective clinical trials.

### FIELD OF DISCLOSURE

The present disclosure relates to new methods to predict the responsiveness of cancer patients to GSPT1 negative modulators and thus assess the efficacy of GSPT1 modulators to treat cancer patients by determining the level of one or more biomarkers in samples of the patients. The present disclosure also relates to applications of these methods, which includes stratifying cancer malignancies, in particular identifying myc-driven cancers, and thereby devising optimized and personalized treatments for these cancer patients, as well as optimizing the selection of patient populations for respective clinical trials.

### BACKGROUND

The increasing number of cancer cases worldwide and the lack of successful therapies is a major concern. Currently there are only few treatments available for only very specific types of cancer, and even those provide typically no guarantee for a cure. While early detection and thus early intervention increases the chances for a successful treatment, a reliable assessment of the cancer and treatment options is still missing.

Tumorigenesis in humans is due to genetic alterations that drive the progressive transformation of normal human cells to abnormally high and uncontrollable levels, which ultimately results in the formation of various malignancies. The extent of deregulated expression differs between patients and patient populations and thus the likelihood of a successful therapeutic response may vary broadly and affects the outcome of a specific treatment and the survival chances of a patient. Yet, there is still a high need for specific and sensitive methods for predicting patient responsiveness and identifying treatment options targeted to a specific patient and specific patient populations, particularly in early stages of the disease. Since early treatment of a specific cancer is absolutely key for survival, a biomarker identifying patients more likely to respond to treatment would be highly beneficial. A reliable assessment of patient status and prediction of patient responsiveness would be highly desirable to be able to select an optimal treatment strategy (and/or tailor an ongoing treatment) and thus increase the chances for survival.

In addition, the ability to predict the efficacy of a treatment would be beneficial in clinical trials for new cancer treatments, as patients could be stratified according to their responsiveness for a participation. This may allow to reduce the number of patients necessary for a clinical study and/or accelerate the time required to complete a clinical development program and result in more meaningful outcomes of a trial.

Applicants have identified specific biomarkers or stratification markers that allow to predict the responsiveness to treatment of cancer, in particular a myc-driven cancer, with one or more GSPT1 negative modulators and thereby distinguish (before or during a treatment) between patients that are more sensitive to such treatment (more responsive patients) and patients that are less responsive to such treatment (less responsive patients). Thus, the use of these biomarkers will allow to devise a therapy specifically targeted to those patients who are more likely to benefit from a GSPT1 negative modulator therapy. This ability to predict the responsiveness to a treatment (with a GSPT1 negative modulator) is beneficial to both patients that are likely to be more responsive as well as patients that are likely to be less responsive.

### SUMMARY OF DISCLOSURE

The present disclosure relates to new methods, which are useful in predicting the responsiveness of a cancer patient to a treatment with one or more GSPT1 negative modulators, and thus are useful in stratifying patients, treating and/or monitoring of a treatment of cancer, such as a myc-driven cancer, with a GSPT1 negative modulator, for example a compound that promotes the degradation of GSPT1. Compounds that promote degradation of, GSPT1 are referred to as GSPT1 Targeted Protein Degraders (TPDs) and include both GSPT1 targeted molecular glue degraders (GSPT1 MSDs) and GSPT1 PROTACs. These methods include determining directly or indirectly the level of one or more biomarkers, such as myc transcription factor markers or surrogate markers thereof, e.g., translation addicted markers as defined herein. In some embodiments these biomarkers include, but are not limited to one or more of L-Myc, N-Myc and c-Myc EIF4EBP1, and EIF4EBP2. In some embodiments, the biomarkers are selected from the group consisting of: L-Myc, N-Myc, EIF4EBP1, and EIF4EBP2. In some cases the biomarker is selected from EIF4EBP1, and EIF4EBP2 and the degree of phosphorylation is determined. The present disclosure also relates to applications of these methods, which includes stratifying malignancies, in particular myc-driven cancers, and thereby devising optimized and personalized therapies for these cancer patients, as well as optimizing the selection of patient populations for respective clinical trials.

In some embodiments, the present disclosure relates to methods to predict and/or monitor the responsiveness of a myc-driven cancer patient to treatment with a GSPT1 negative modulator.

In some embodiments, the present disclosure relates to methods to predict and/or monitor the effectiveness of a GSPT1 negative modulator in the treatment of a myc-driven cancer as defined herein. In some embodiments, the present disclosure relates to methods to assess and monitor the progress of a treatment of a myc-driven cancer as defined herein with a GSPT1 negative modulator.

Described herein is a method of treating a patient suffering from a Myc-driven tumor, comprising: (a) determining the expression level of one or more Myc transcription factor biomarkers in a biological sample obtained from the patient; and (b) treating the patient with a treatment regimen comprising administering a therapeutically effective amount of a GSPT1 negative modulator if the expression level of the one more Myc transcription factor biomarkers is greater than a reference level for the one more Myc transcription factor biomarkers.

In various embodiments: the biological sample comprises tumor cells or tumor nucleic acid; the step of determining comprises acquiring data; the step of determining comprises obtaining a biological sample and measuring expression or having a biological sample obtained and having expression measured; the tumor nucleic acid is tumor DNA or tumor RNA; the step of determining expression level comprising measuring the copy number a gene encoding a Myc transcription factor biomarker; the one or more Myc transcription factor biomarkers are selected from the group consisting of: L-Myc, N-Myc, c-Myc, EIF4EBP1 and EIF4EBP2; the method further comprises treating the patient with a treatment regime other than administering a GSPT1 negative modulator if the expression level of the one more Myc transcription factor biomarkers is not greater than a reference level for the one more Myc transcription factor biomarkers; the GSPT1 negative modulator is a targeted protein degraders that promotes degradation of GSPT1 protein in cells; the GSPT1 negative modulator is a molecular glue degrader; the biological sample is obtained before the patient is treated with a GSPT1 negative modulator; and the biological sample is obtained after the patient is treated with a GSPT1 negative modulator.

Also described herein is a method of treating a patient suffering from a Myc-driven tumor, comprising: (a) determining the phosphorylation level of one or more of EIF4EBP1 and EIF4EBP2 in a biological sample obtained from the patient; (b) treating the patient with a treatment regimen comprising administering a therapeutically effective amount of a GSPT1 negative modulator if the phosphorylation level of one or more of EIF4EBP1 and EIF4EBP2 is greater than a reference level for the one more Myc transcription factor biomarkers.

In various embodiments: the step of determining comprises acquiring data; the step of determining comprises obtaining a biological sample and measuring expression or having a biological sample obtained and having expression measured; the method further comprises treating the patient with a treatment regime other than administering a GSPT1 negative modulator if the expression level of the one more Myc transcription factor biomarkers is not greater than a reference level for the one more Myc transcription factor biomarkers; the GSPT1 negative modulator is a targeted protein degraders that promotes degradation of GSPT1 protein in cells; the GSPT1 negative modulator is a molecular glue degrader; the biological sample is obtained before the patient is treated with a GSPT1 negative modulator; the biological sample is obtained after the patient is treated with a GSPT1 negative modulator.

Also described is a method of treating a patient suffering from a Myc-driven tumor, comprising: (a) identifying a patient having a Myc-driver tumor; and (b) treating the patient with a treatment regimen comprising administering a therapeutically effective amount of a GSPT1 negative modulator.

Also described is a method of treating a patient suffering from a Myc-driven tumor, comprising administering a therapeutically effective amount if a GSPT1 negative modulator.

Also described is a method for determining a treatment for a patient suffering from a Myc-driven tumor from whom a biological sample was obtained and the expression level of one or more Myc transcription factor biomarkers was measured, the method comprising selecting a treatment regimen from the group consisting of: (a) treating with a therapeutically effective amount of a GSPT1 negative modulator if the expression level of the one more Myc transcription factor biomarkers is greater than a reference level for the one more Myc transcription factor biomarkers; (b) treating with a therapeutic agent that other than a GSPT1 negative modulator if the expression level of the one more Myc transcription factor biomarkers is not greater than a reference level for the one more Myc transcription factor biomarkers.

In any of the forgoing methods: the Myc-driven cancer is selected from the group consisting of: breast cancer, small cell lung carcinoma, non-small cell lung carcinoma, a neuroendocrine cancer, acute myelogenous leukemia, lymphoma, and multiple myeloma.

In various embodiments of the forgoing methods the GSPT1 negative modulator is a compound or a pharmaceutically acceptable salt or stereoisomer thereof of formula I:

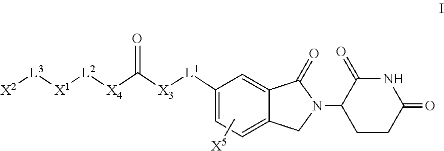

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

In various embodiments of the forgoing methods X⁴—CO—X³— is —NH—CO—NH— or —NH—CO—O— or —CH₂—CO—NH— or —CH₂—CO—O—.

In various embodiments of the forgoing methods the GSPT1 modulator is a compound or a pharmaceutically acceptable salt or stereoisomer thereof of formula II,

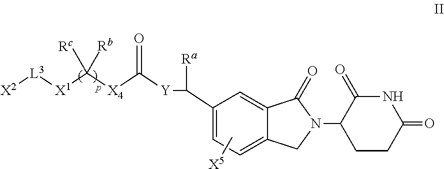

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is N or O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen; and
p is 0, 1, 2.

In various embodiments of the forgoing methods, GSPT1 negative modulator is a compound of formula 1 is a compound or a pharmaceutically acceptable salt or stereoisomer thereof of formula Va:

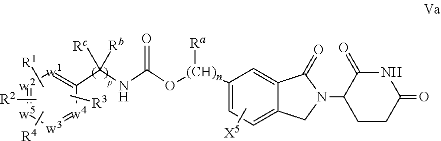

wherein
w¹, w², w³, w⁴, w⁵ are independently of each other selected from C and N, with the proviso that at least three of w¹, w² w³ w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.

In various embodiments of the forgoing methods, the GSPT1 negative modulator is selected from any of Compounds 1-160, 201-440 and 501 to 573 and pharmaceutically acceptable salts thereof.

In various embodiments of the forgoing methods the GSPT1 negative modulator is selected from any of Compounds 1-160, 29.

In various embodiments of the forgoing methods, the GSPT1 negative modulator is selected from any of:

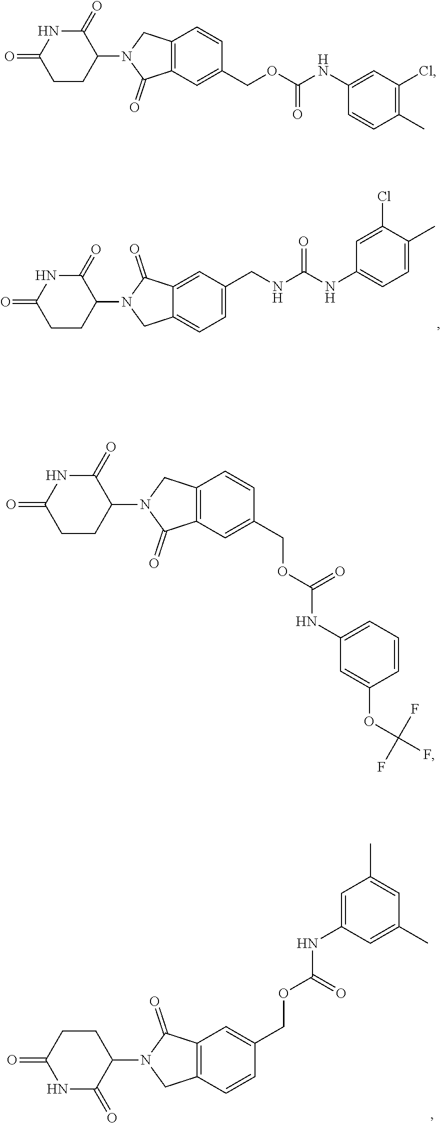 [SMILES: Cc1ccc(NC(=O)OCc2ccc3c(c2)C(=O)N(C2CCC(=O)NC2=O)C3)cc1Cl ; Cc1ccc(NC(=O)NCc2ccc3c(c2)C(=O)N(C2CCC(=O)NC2=O)C3)cc1Cl ; O=C1CCC(N2Cc3ccc(COC(=O)Nc4cccc(OC(F)(F)F)c4)cc3C2=O)C(=O)N1 ; Cc1cc(C)cc(NC(=O)OCc2ccc3c(c2)C(=O)N(C2CCC(=O)NC2=O)C3)c1]

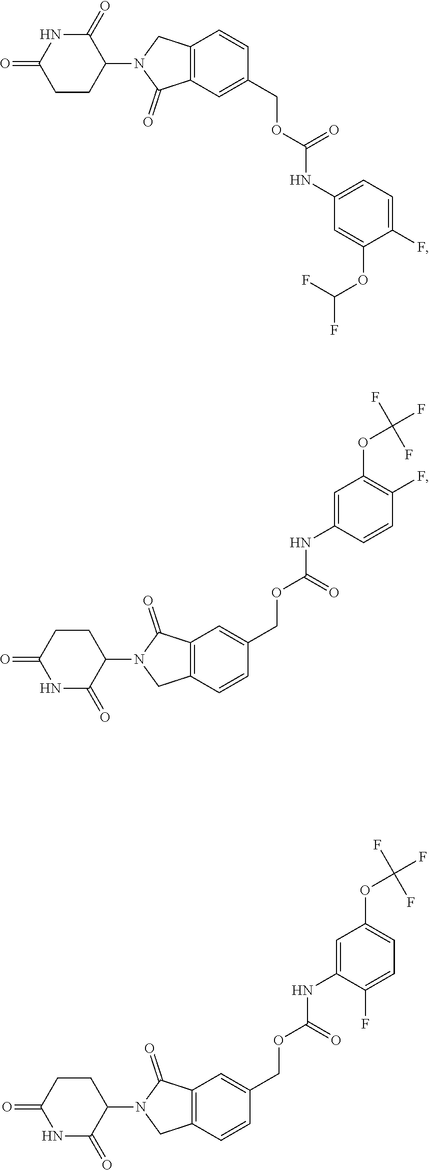 [SMILES: O=C1CCC(N2Cc3ccc(COC(=O)Nc4ccc(F)c(OC(F)F)c4)cc3C2=O)C(=O)N1 ; O=C1CCC(N2Cc3ccc(COC(=O)Nc4ccc(F)c(OC(F)(F)F)c4)cc3C2=O)C(=O)N1 ; O=C1CCC(N2Cc3ccc(COC(=O)Nc4cc(OC(F)(F)F)ccc4F)cc3C2=O)C(=O)N1]

and pharmaceutically acceptable salts thereof.

In some embodiments, the present disclosure relates to an in vitro method to determine or assess the responsiveness of a cancer patient to a treatment with at least one GSPT1 negative modulator, comprising the steps of (i) obtaining a cancerous sample from the patient, (ii) determining the level of one or more biomarkers selected from a myc transcription factor marker or surrogate marker thereof, such as a translation addicted marker, e.g. one or more of EIF4EBP1, EIF4EBP2, L-Myc, N-Myc and c-Myc, or combinations thereof in the cancerous sample, (iii) comparing the level of the one or more biomarkers determined in step (ii) with the level of one or more reference markers determined in a control sample, and (iv) identifying the patient as having an increased or decreased responsiveness to the treatment if the level of the one or more biomarkers in the cancerous sample is altered in comparison to the level of the one or more reference markers in the control sample.

Once the expression level of one or more of L-Myc, N-Myc, c-Myc EIF4EBP1 and EIF4EBP2 is measured in the sample obtained from the subject, the measured expression level is compared to a reference level (e.g., with one or both of the reference level and the measured level normalized, for example to the expression of one or more housekeeping genes). In some, aspects, the expression level of at least one, two or three (if not, four or five) of L-Myc, N-Myc, c-Myc EIF4EBP1 and EIF4EBP2 are measured. In some cases, the reference level is the corresponding tissue-matched expression level of one or more of L-Myc, N-Myc, c-Myc EIF4EBP1 and EIF4EBP2 in a population of subjects not suffering from cancer. In some cases, the phosphorylation of one or more of EIF4EBP1 and EIF4EBP2 is measured and compared to a reference level of phosphorylation. The reference level of phosphorylation the corresponding tissue-matched phosphorylation preferably determined in a population of subjects not suffering from cancer.

In some aspects of the methods described herein, the expression or phosphorylation level that is measured may be the same as a reference level, e.g., a control level or a cut off level or a threshold level, or may be increased or decreased relative to a reference level, e.g., control level or a cut off level or a threshold level. In exemplary aspects, the reference expression level(s) of the gene(s), protein(s), RNA, or the expression level(s) is/are level(s) of a subject known to not have a tumor. The reference level is determined in non-cancerous tissue of the same type as the tumor. In the case of phosphorylation, the degree of phosphorylation is compared to a reference level of phosphorylation for the marker in tissue-matched non-cancer (non-tumor) sample(s).

In some aspects, the reference level is that of a reference subject or population of subjects which may be a matched control of the same species, gender, ethnicity, age group, smoking status, BMI, current therapeutic regimen status, medical history, or a combination thereof, but differs from the subject being diagnosed or from whom a sample was obtained in that the reference does not suffer from the disease in question or is not at risk for the disease. alternative aspects, the reference expression level(s) of the gene(s), protein(s), RNA, or the expression level(s) or phosphorylation levels protein(s) is/are level(s) of a subject known to have a tumor. In exemplary aspects, as further described herein, the measured level is compared to both a reference level of a subject known to not have a tumor and a reference level of a subject known to have a tumor. In exemplary aspects, the reference level is the mean of a population of expression levels for the corresponding gene. For example, the reference level for an L-Myc expression level of the sample is the mean L-Myc expression level among a population, the reference level for an N-Myc expression level of the sample is the mean L-Myc expression level among a population, the reference level for a c-Myc expression level of the sample is the mean c-Myc expression level among a population, the reference level for an EIF4EBP1, expression level of the sample is the mean EIF4EBP1 expression level among a population, and the reference level for an EIF4EBP2 expression level of the sample is the mean EIF4EBP2 expression level among a population.

In some embodiments, the cancer is a myc-driven cancer. In some embodiments, the cancer is a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), lymphoma, Hodgkin's lymphoma, non-Hodgkin's lymphomas and multiple myeloma (MM).

In some embodiments, the biomarker is N-Myc and/or L-Myc and/or c-Myc and the cancer is AML. In some embodiments, the biomarker is N-Myc and/or L-Myc and the cancer is MM. In some embodiments, the biomarker is N-Myc and the cancer is AML. In some embodiments, the biomarker is N-Myc and the cancer is MM. In some embodiments, the biomarker is c-Myc and the cancer is lymphoma In some embodiments, the cancer is a myc-driven cancer. In some embodiments, the cancer is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments, the biomarker is EIF4EBP1 and/or EIF4EBP2 and/or c-Myc and the cancer is breast cancer. In some embodiment, the biomarker is EIF4EBP1 and/or EIF4EBP2 and/or L-Myc and/or N-Myc (i.e., high expression) and the cancer is SCLC.

In some embodiments, the biomarker is N-Myc (i.e., high expression) and the cancer is a neuroendocrine cancer, for example, neuroendocrine prostate cancer (for example, NEPC (castration-resistant neuroendocrine prostate cancer)) and lung neuroendocrine tumors (Lu-NETs). In some embodiments, the biomarker is L-Myc and/or N-Myc (i.e., high expression) and the cancer is NSCLC. In some embodiments, the biomarker is N-Myc (i.e., high expression) and the cancer is gastric cancer or liver cancer.

In some embodiments, the present disclosure relates to a method of treating a cancer patient with a therapeutically effective amount of a GSPT1 negative modulator comprising (i) obtaining a cancerous sample from the patient, (ii) determining the level of one or more biomarkers selected from myc transcription factor marker or surrogate marker thereof, such as a translation addicted marker, e.g. one or more of EIF4EBP1, EIF4EBP2, L-Myc, N-Myc and c-Myc, or combinations thereof, such as EIF4EBP1 and/or EIF4EBP2 and/or L-Myc, in the cancerous sample, (iii) comparing the level of the one or more biomarkers determined in step (ii) with the level of one or more reference markers determined in a control sample, (iv) identifying the patient as having an increased or decreased responsiveness to a treatment with a GSPT1 negative modulator if the level of the one or more biomarkers in the cancerous sample is altered in comparison to the level of the one or more reference markers in the control sample, and (v) administering to the patient having an increased responsiveness to the treatment with a GSPT1 negative modulator the therapeutically effective amount of a GSPT1 negative modulator.

In some embodiments, the present disclosure relates to a one or more biomarkers selected from a translation addicted marker and combinations thereof, such as EIF4EBP1 and/or EIF4EBP2 and/or L-Myc and/or N-Myc for use in the determination of the responsiveness of a cancer patient to a treatment with a GSPT1 negative modulator, wherein a different level of the one or more biomarkers in a cancerous sample obtained from said patient compared to a control sample is indicative that said patient has an increased or decreased likelihood of responsiveness to the treatment with a GSPT1 negative modulator.

In some embodiments, the GSPT1 negative modulator is selected from Compounds 1-160, 201-443 and 501-573. In some embodiments the GSPT1 negative modulator is Compound 8 or Compound 210 or Compound 345.

Described herein is an in vitro method to determine or assess the responsiveness of a cancer patient to a treatment with at least one GSPT1 modulator, comprising the steps of (i) obtaining a cancerous sample from the patient, (ii) determining the level of one or more biomarkers selected from a myc transcription factor marker or surrogate marker thereof, such as a translation addicted marker, e.g. one or more of EIF4EBP1, pEIF4EBP2, L-myc, N-myc and C-myc, or combinations thereof in the cancerous sample, (iii) comparing the level of the one or more biomarkers determined in step (ii) with the level of one or more reference markers determined in a control sample, and (iv) identifying the patient as having an increased or decreased responsiveness to the treatment if the level of the one or more biomarkers in the cancerous sample is altered in comparison to the level of the one or more reference markers in the control sample.

Also described is a method of treating a cancer patient with a therapeutically effective amount of a GSPT1 modulator comprising: (i) obtaining a cancerous sample from the patient, (ii) determining the level of one or more biomarkers selected from myc transcription factor marker or surrogate marker thereof, such as a translation addicted marker, e.g. one or more of EIF4EBP1, EIF4EBP2, L-myc, N-myc and C-myc, or combinations thereof, such as EIF4EBP1 and/or EIF4EBP2 and/or L-myc, in the cancerous sample, (ii) comparing the level of the one or more biomarkers determined in step (ii) with the level of one or more reference markers determined in a control sample, (iv) identifying the patient as having an increased or decreased responsiveness to a treatment with a GSPT1 modulator if the level of the one or more biomarkers in the cancerous sample is altered in comparison to the level of the one or more reference markers in the control sample, and (V) administering to the patient having an increased responsiveness to the treatment with a GSPT1 modulator the therapeutically effective amount of a GSPT1 modulator.

In various embodiments: The cancer is a solid tumor cancer, such as breast cancer, colorectal cancer, lung cancer, e.g. SCLC, NSCLC, liver cancer, neuroendocrine cancer, such as neuroendocrine prostate cancer, e.g. castration-resistant neuroendocrine prostate cancer, and lung neuroendocrine tumors, stomach cancer, pancreatic cancer, gastric cancer, esophageal cancer, bladder cancer, skin cancer, and head and neck cancer; the biomarker is EIF4EBP1 and the cancer is breast cancer; the biomarker is L-myc and the cancer is SCLC; the biomarker is N-myc and the cancer is a neuroendocrine cancer; the control sample is obtained (i) from a healthy subject, or (ii) from a non-cancerous sample obtained from the cancer patient, or (iii) from a cancerous biological sample obtained from the patient taken at an earlier time point, or (iv) from a cancer patient other than the cancer patient of which the cancerous sample has been obtained, which has previously been determined to be a responder or a non-responder; and the cancerous sample is obtained before the cancer patient is subjected to the treatment with a GSPT1 modulator or during the cancer patient is subjected to the treatment with a GSPT1 modulator or after the cancer patient is subjected to the treatment with a GSPT1 modulator.

Also described is the use of one or more biomarkers selected from a translation addicted marker and combinations thereof, such as EIF4EBP1 and/or EIF4EBP2 and/or L-myc, in the determination of the responsiveness of a cancer patient to a treatment with a GSPT1 modulator, wherein an increased level of the one or more biomarkers in a cancerous sample obtained from said patient compared to a control sample is indicative that said patient has an increased likelihood of responsiveness to the treatment with a GSPT1 modulator and wherein a decreased level of the one or more biomarkers in a cancerous sample obtained from said patient compared to a control sample is indicative that said patient has a decreased likelihood of responsiveness to the treatment with a GSPT1 modulator.

In any of the forgoing embodiments, the GSPT1 modulator is a compound or a pharmaceutically acceptable salt or stereoisomer thereof of formula I:

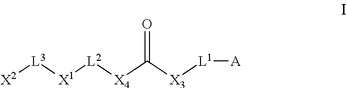

wherein
A is

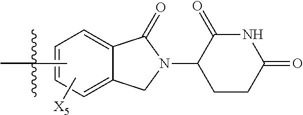

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

In any of the forgoing embodiments, the GSPT1 modulator is a compound or a pharmaceutically acceptable salt or stereoisomer thereof of formula Va and IVb, Va and Vb, VIa and VIb, or VIIa and VIIb,

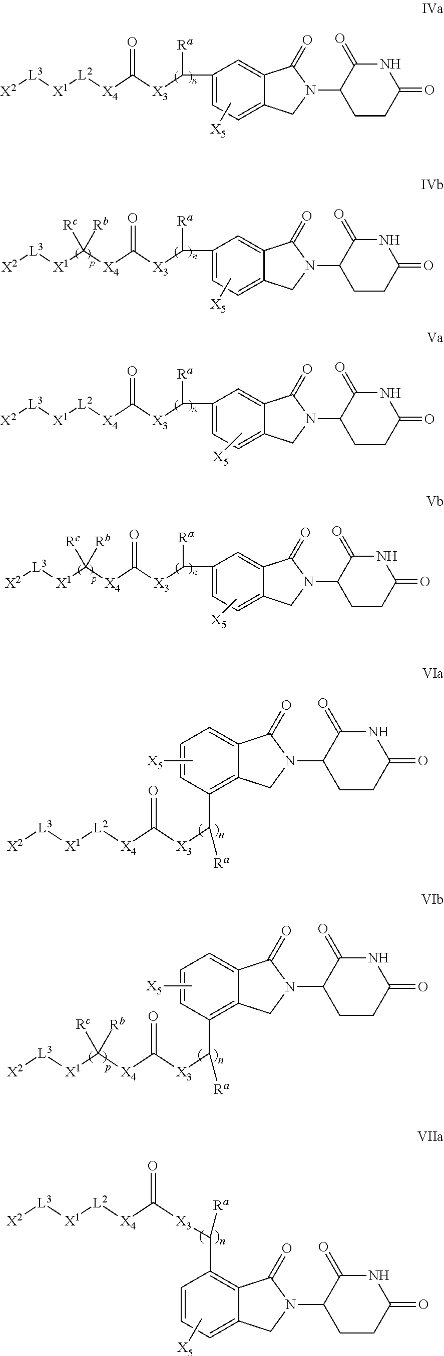

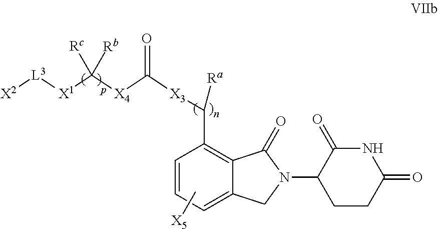

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen;
R^{a} is a H or C₁₋₄ alkyl;
R^{b}, R^{c} are independently of each other H, C₁₋₄ alkyl, such as methyl, ethyl, or halogen, such as F;
n is 0, 1, 2;
p is 0, 1, 2.

In any of the forgoing embodiments, X⁴—CO—X³— is —NH—CO—NH— or —NH—CO—O— or —CH₂—CO—NH— or —CH₂—CO—O—.

In any of the forgoing embodiments, the GSPT1 modulator is a compound or a pharmaceutically acceptable salt or stereoisomer, thereof of formula VIII

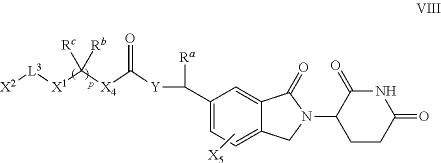

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁵ is H, linear or branched C₁₋₆ alkyl, —C₁₋₄ alkoxy, —CN, halogen, CF₃, CHF₂, CMeF₂, OCF₃, OCHF₂;
Y is N or O;
R^{a} is a H or C₁₋₄ alkyl;
R^{b}, R^{c} are independently of each other H, C₁₋₄ alkyl, such as methyl, ethyl, or halogen, such as F;
L³ is a covalent bond, —O—, —C₁₋₄ alkoxy or C₁₋₆ alkyl, which is unsubstituted or substituted with one or more of C₁₋₄ alkyl, halogen;
p is 0, 1, 2.

In any of the forgoing embodiments, the GSPT1 modulator is a compound or a pharmaceutically acceptable salt or stereoisomer, thereof of formula X

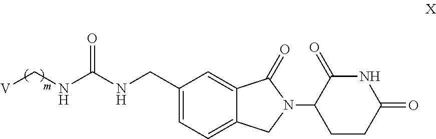

wherein
m is 0, 1, 2 or 3, and
V is selected from

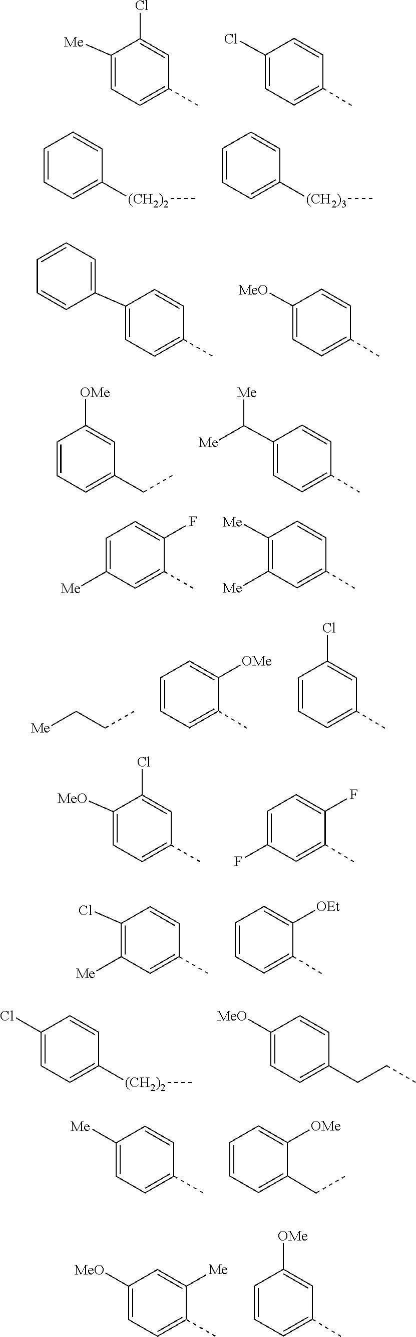

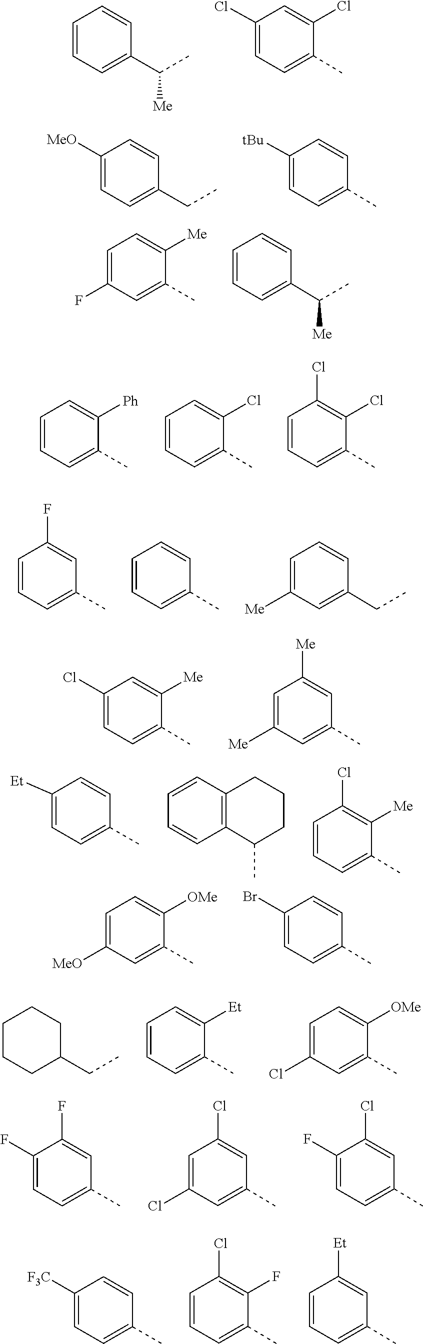

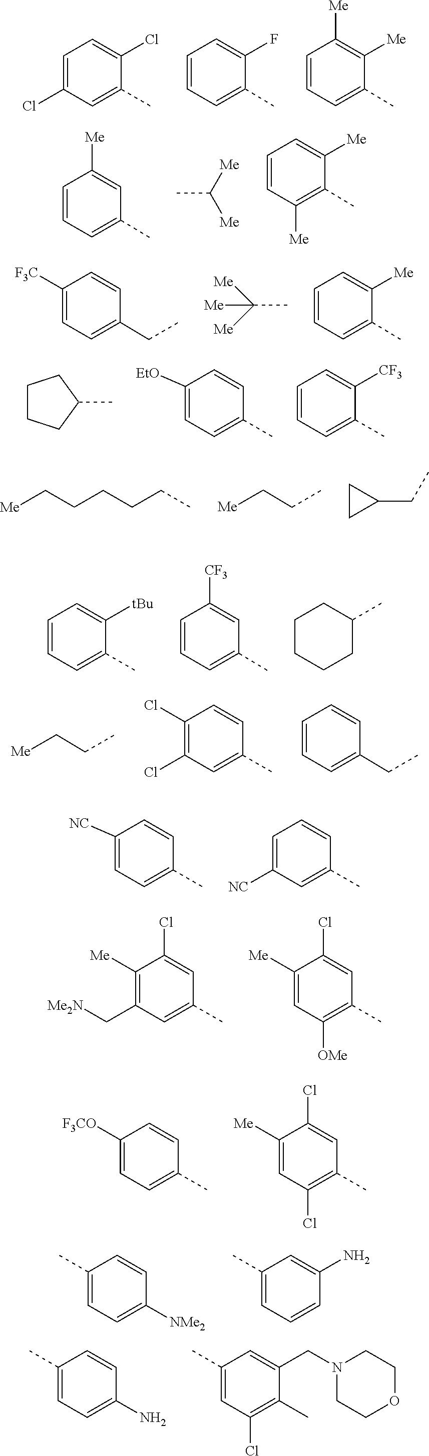

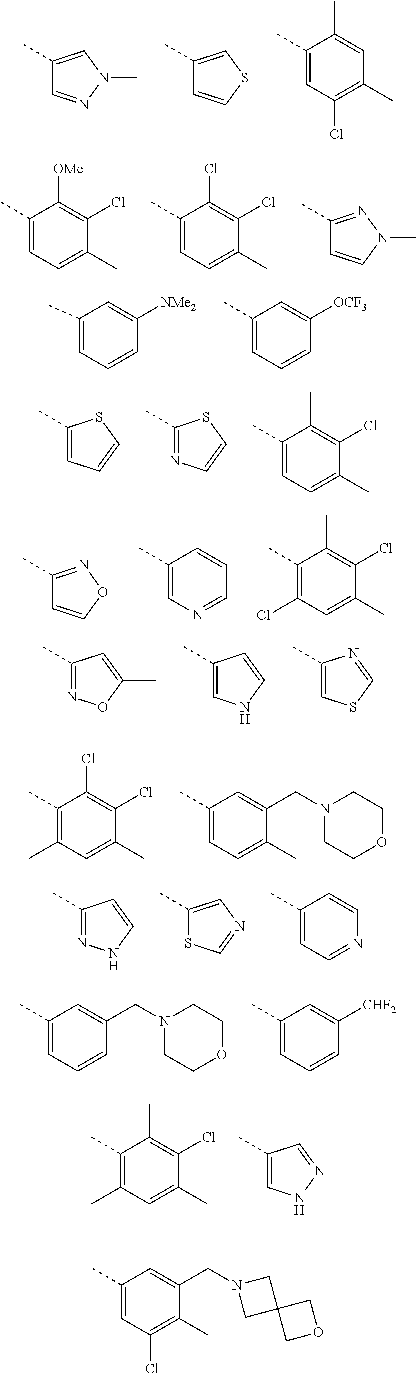

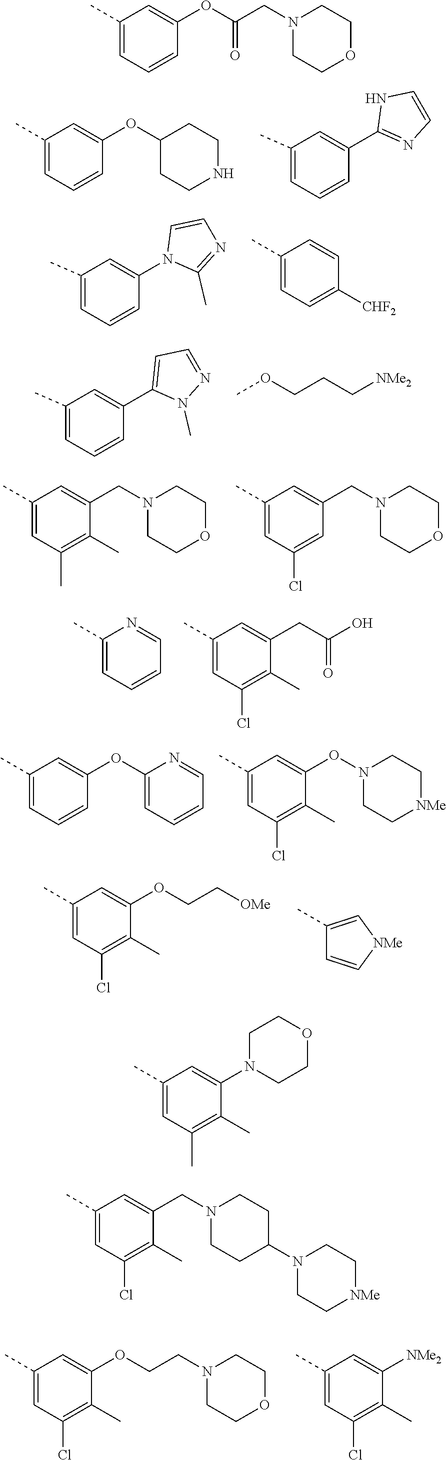

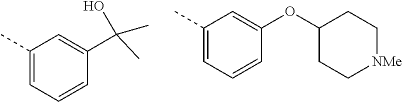

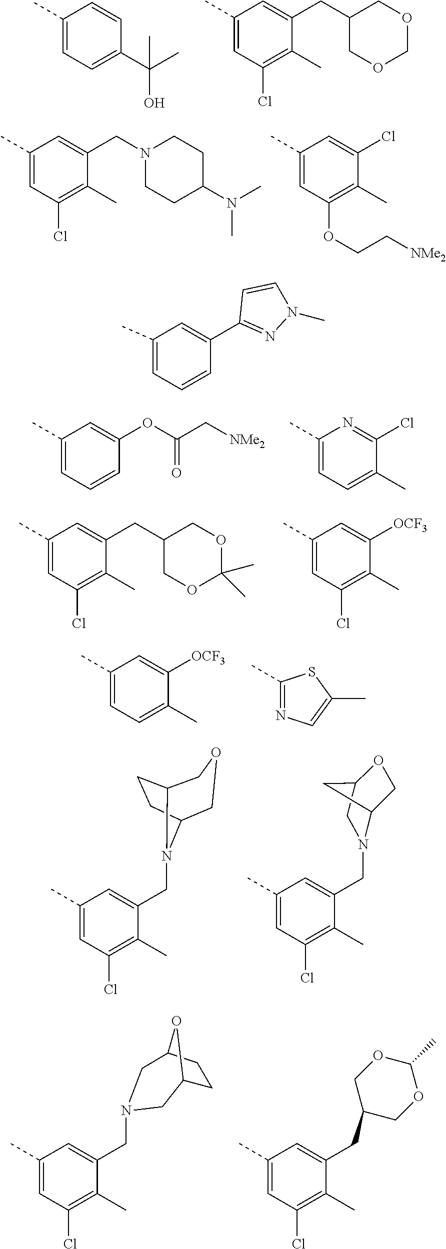

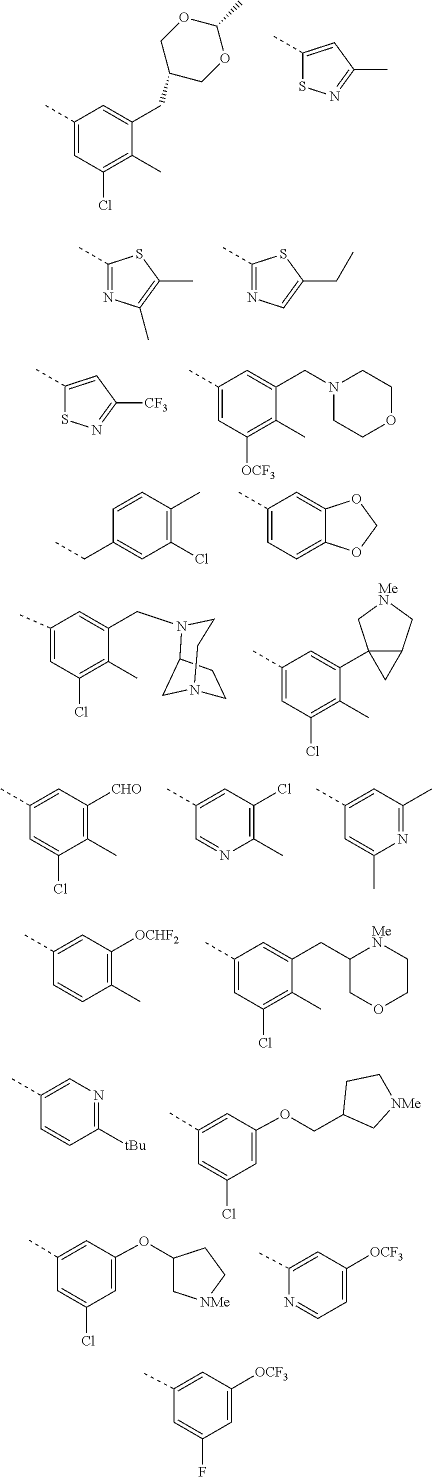

In any of the forgoing embodiments, the GSPT1 modulator is a compound or a pharmaceutically acceptable salt or thereof of formula XII

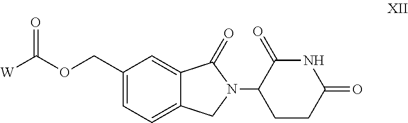

wherein W is selected from

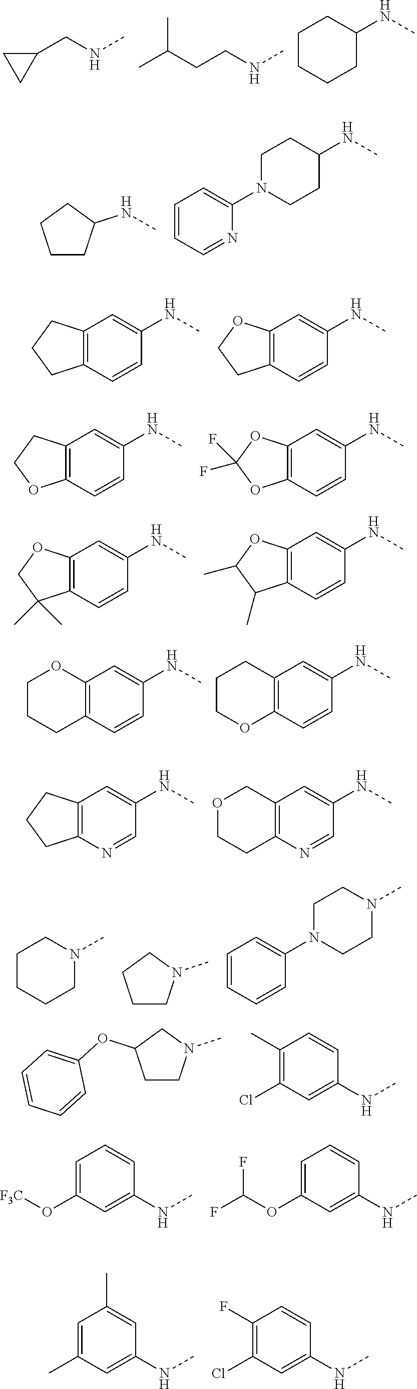

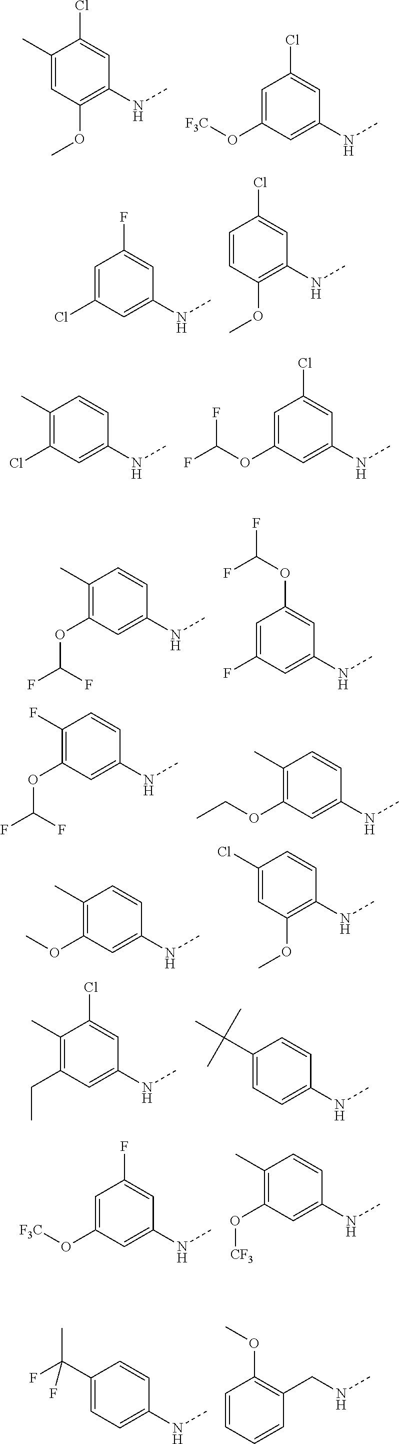

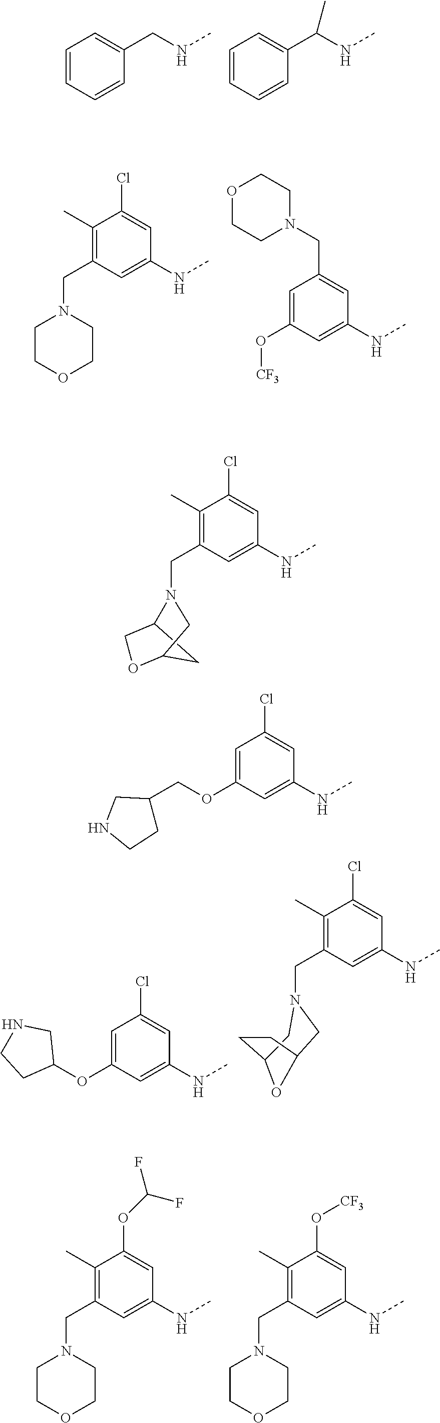

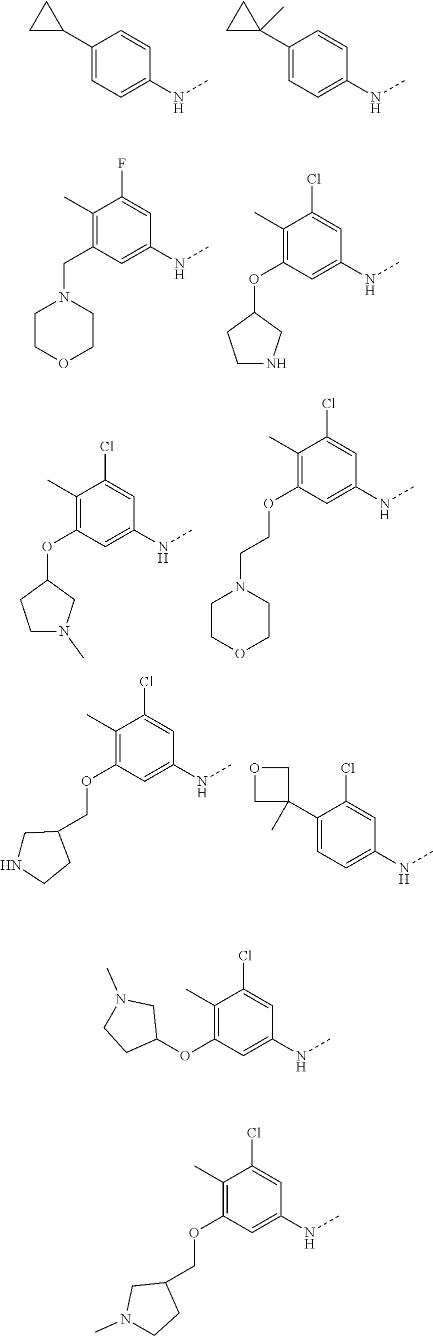

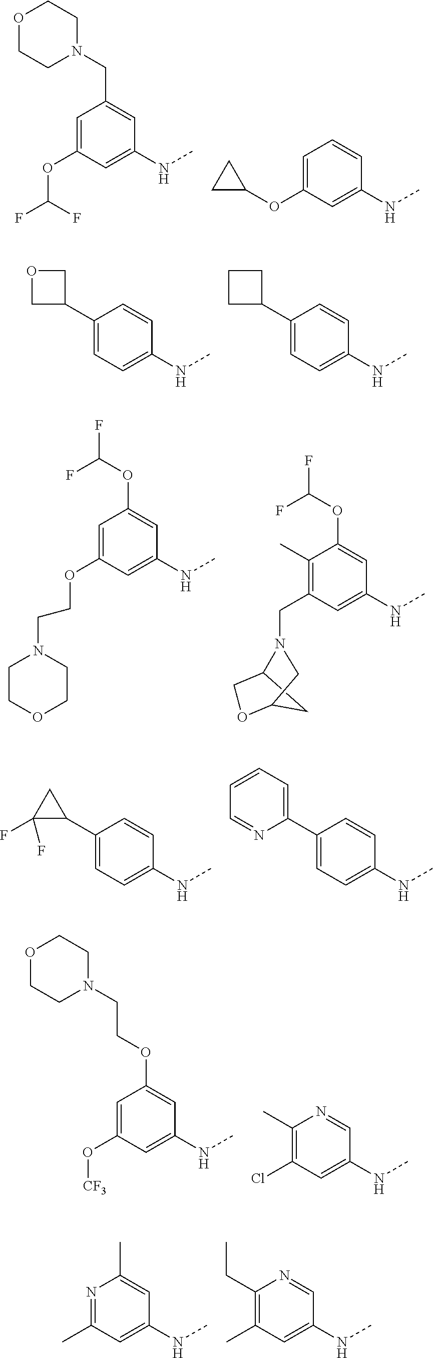

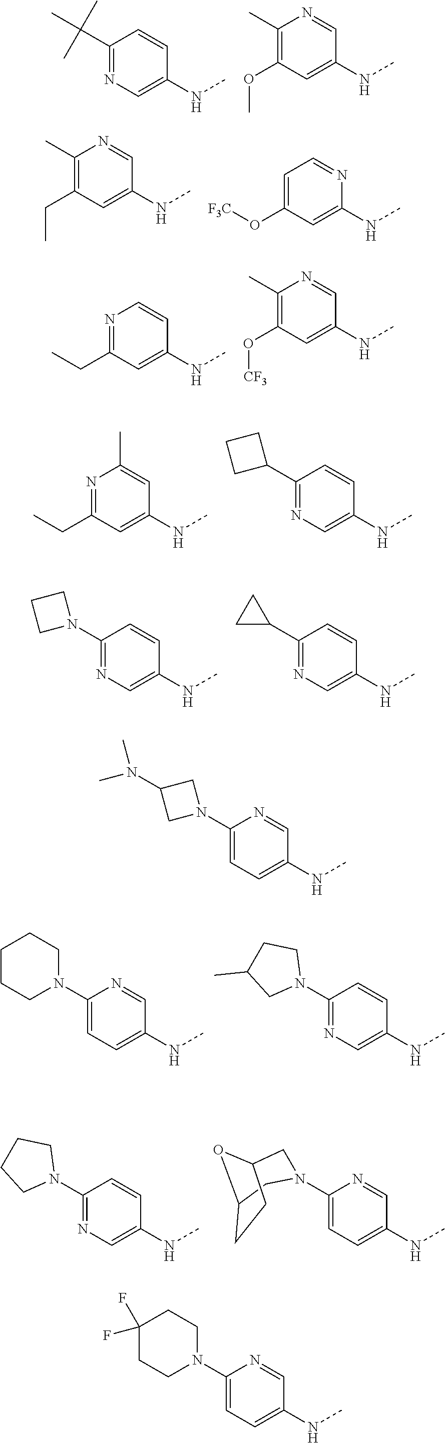

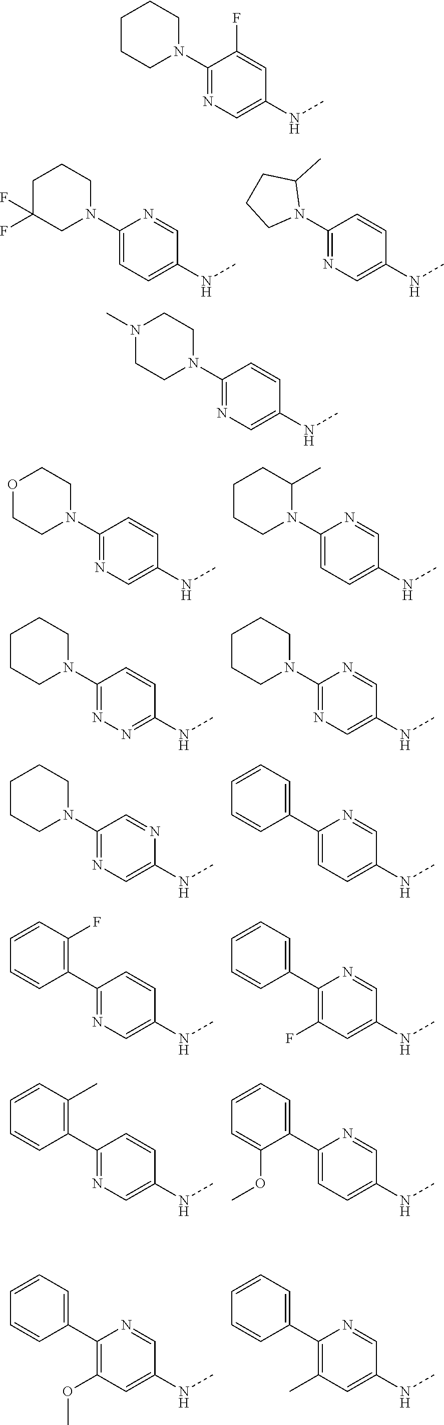

In any of the forgoing embodiments, the GSPT1 modulator is a compound or a pharmaceutically acceptable salt or stereoisomer thereof of formula XIII or XIIIa, XIIIb, XIIIc

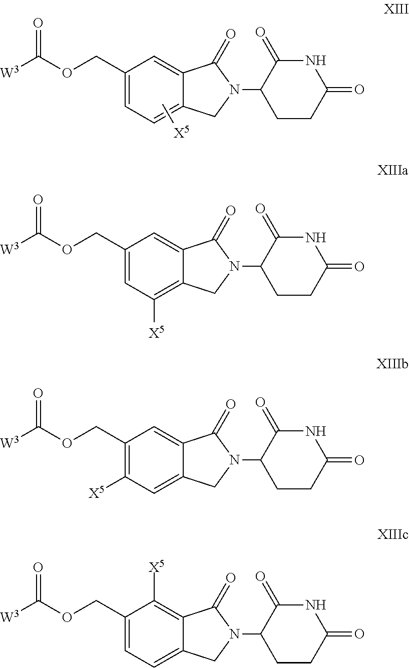

wherein X⁵ is linear or branched C₁₋₆ alkyl, —C₁₋₄ alkoxy, —CN, halogen, CF₃, CHF₂, CMeF₂, OCF₃, OCHF₂, in particular C₁₋₄ alkyl, such as methyl, —C₁₋₄ alkoxy, such as —OMe, —CN, halogen, such as F, Cl, Br; and W³ is selected from

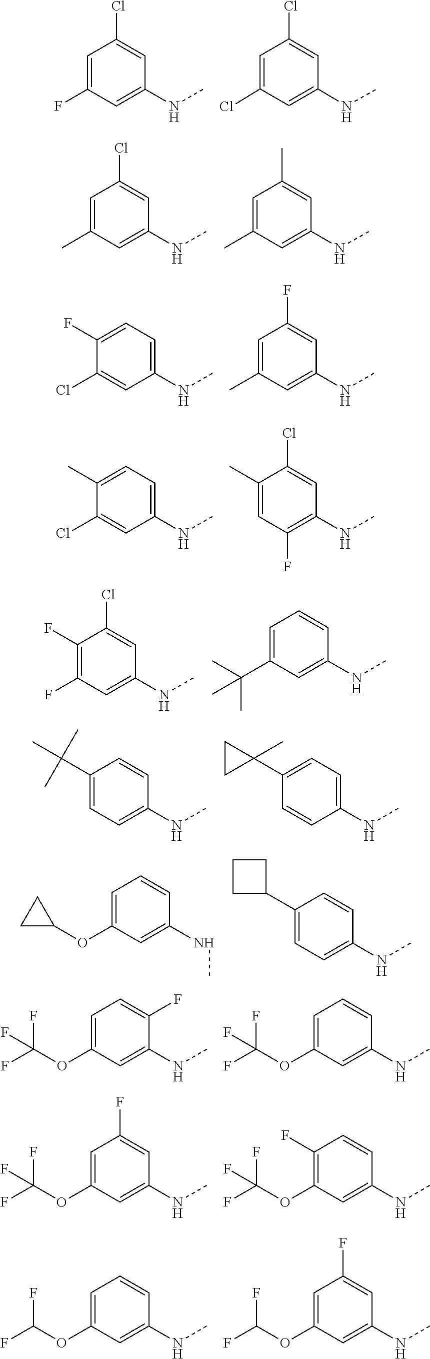

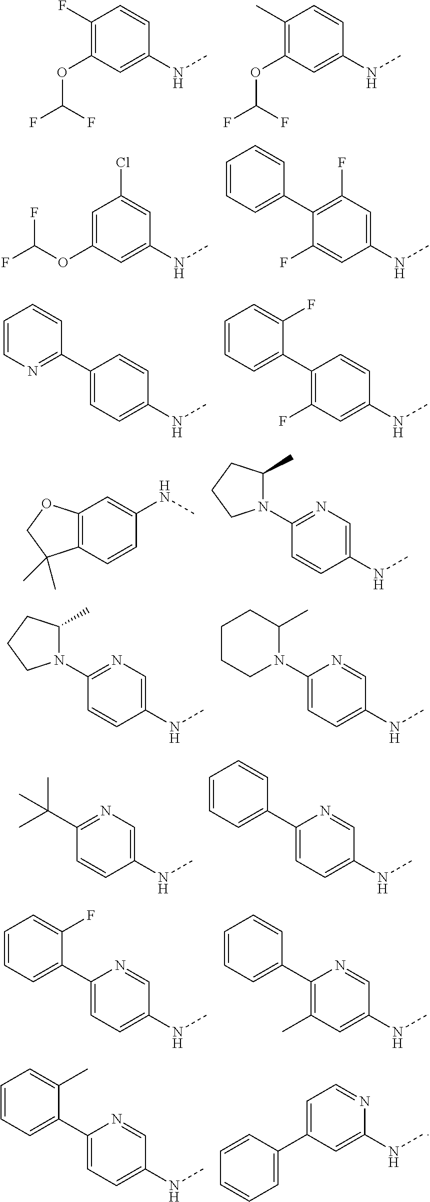

Described herein is an in vitro method to determine or assess the responsiveness of a cancer patient to a treatment with at least one GSPT1 modulator, comprising the steps of
(i) obtaining a cancerous sample from the patient,
(ii) determining the level of one or more biomarkers selected from a myc transcription factor marker or surrogate marker thereof, such as a translation addicted marker, e.g. one or more of EIF4EBP1, pEIF4EBP2, L-myc, N-myc and C-myc, or combinations thereof in the cancerous sample,
(iii) comparing the level of the one or more biomarkers determined in step (ii) with the level of one or more reference markers determined in a control sample, and
(iv) identifying the patient as having an increased or decreased responsiveness to the treatment if the level of the one or more biomarkers in the cancerous sample is altered in comparison to the level of the one or more reference markers in the control sample.

Also described herein is a method of treating a cancer patient with a therapeutically effective amount of a GSPT1 modulator comprising:
(i) obtaining a cancerous sample from the patient,
(ii) determining the level of one or more biomarkers selected from myc transcription factor marker or surrogate marker thereof, such as a translation addicted marker, e.g. one or more of EIF4EBP1, EIF4EBP2, L-myc, N-myc and c-myc, or combinations thereof, such as EIF4EBP1 and/or EIF4EBP2 and/or L-myc, in the cancerous sample,
(iii) comparing the level of the one or more biomarkers determined in step (ii) with the level of one or more reference markers determined in a control sample,
(iv) identifying the patient as having an increased or decreased responsiveness to a treatment with a GSPT1 modulator if the level of the one or more biomarkers in the cancerous sample is altered in comparison to the level of the one or more reference markers in the control sample, and
(v) administering to the patient having an increased responsiveness to the treatment with a GSPT1 modulator the therapeutically effective amount of a GSPT1 modulator.

In various embodiments: the cancer is a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM); the biomarker is N-myc and the cancer is AML; the biomarker is N-myc and the cancer is MM; the biomarker is L-myc and the cancer is AML; the biomarker is L-myc and the cancer is MM; the control sample is obtained (i) from a healthy subject, or (ii) from a non-cancerous sample obtained from the cancer patient, or (iii) from a cancerous biological sample obtained from the patient taken at an earlier time point, or (iv) from a cancer patient other than the cancer patient of which the cancerous sample has been obtained, which has previously been determined to be a responder or a non-responder; and the cancerous sample is obtained before the cancer patient is subjected to the treatment with a GSPT1 modulator or during the cancer patient is subjected to the treatment with a GSPT1 modulator or after the cancer patient is subjected to the treatment with a GSPT1 modulator.

Also described is the use of one or more biomarkers selected from a translation addicted marker and combinations thereof, such as EIF4EBP1 and/or EIF4EBP2 and/or L-myc, in the determination of the responsiveness of a cancer patient to a treatment with a GSPT1 modulator, wherein an increased level of the one or more biomarkers in a cancerous sample obtained from said patient compared to a control sample is indicative that said patient has an increased likelihood of responsiveness to the treatment with a GSPT1 modulator and wherein a decreased level of the one or more biomarkers in a cancerous sample obtained from said patient compared to a control sample is indicative that said patient has a decreased likelihood of responsiveness to the treatment with a GSPT1 modulator.

In various embodiment of the forgoing methods and uses: the use of claim 10 wherein the GSPT1 modulator is a compound or a pharmaceutically acceptable salt or stereoisomer thereof of formula I:

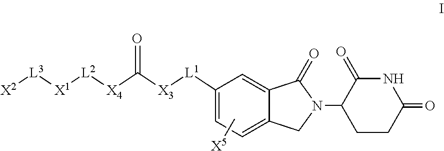

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

The method of claim 11, wherein X⁴—CO—X³— is —NH—CO—NH— or —NH—CO—O— or —CH₂—CO—NH— or —CH₂—CO—O—.

The method of claim 11, wherein the GSPT1 modulator is a compound or a pharmaceutically acceptable salt or stereoisomer thereof of formula II,

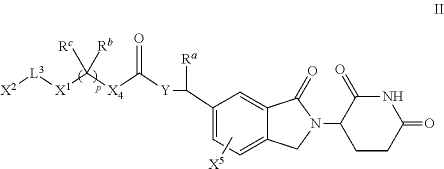

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
   or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is N or O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen;
p is 0, 1, 2.

The method of claim 11 wherein the GSPT1 modulator is a compound or a pharmaceutically acceptable salt or stereoisomer thereof of formula IV

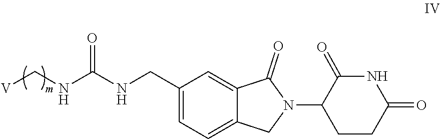

wherein
m is 0, 1, 2 or 3, and
V is selected from

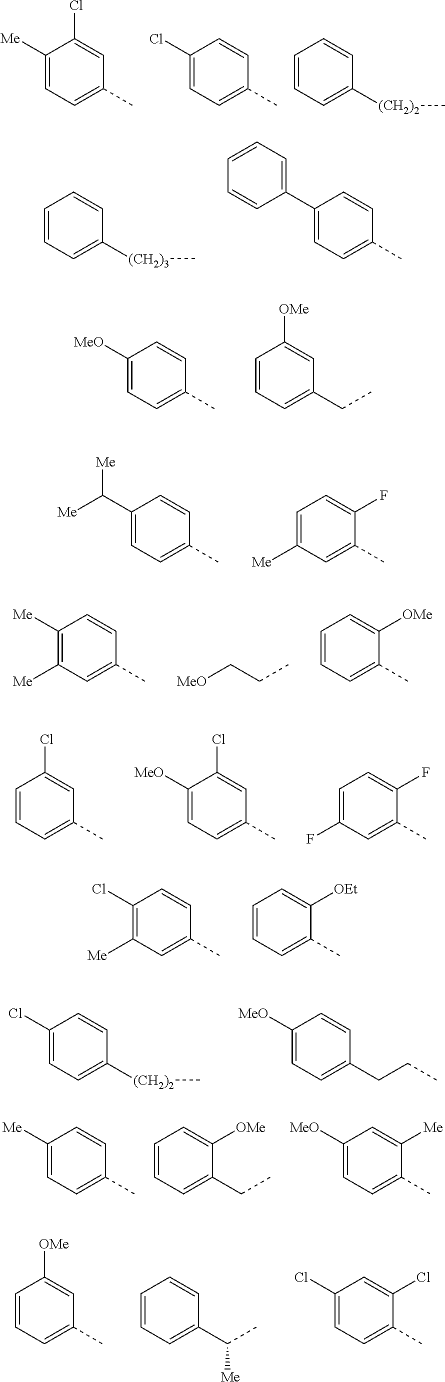

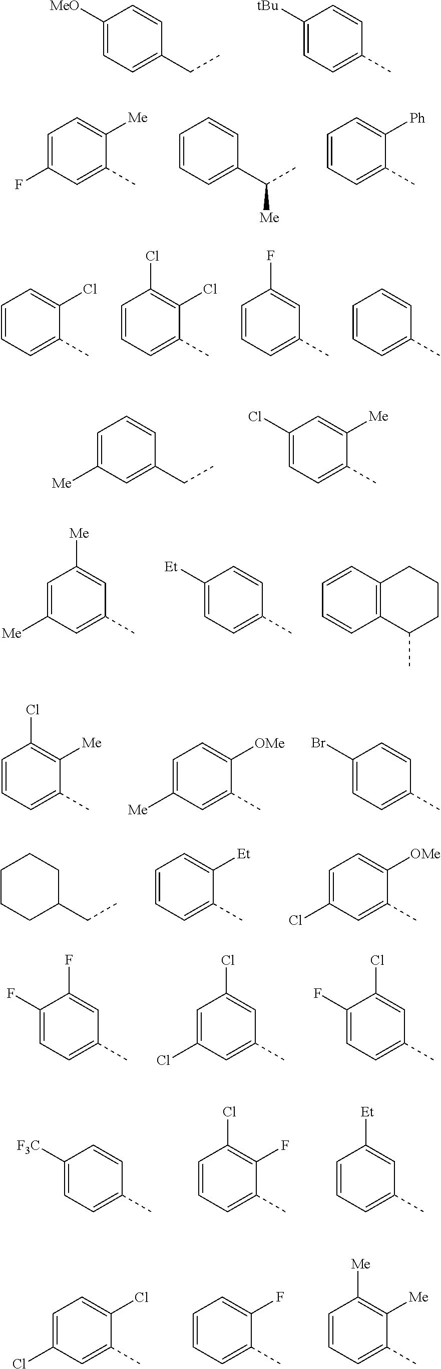

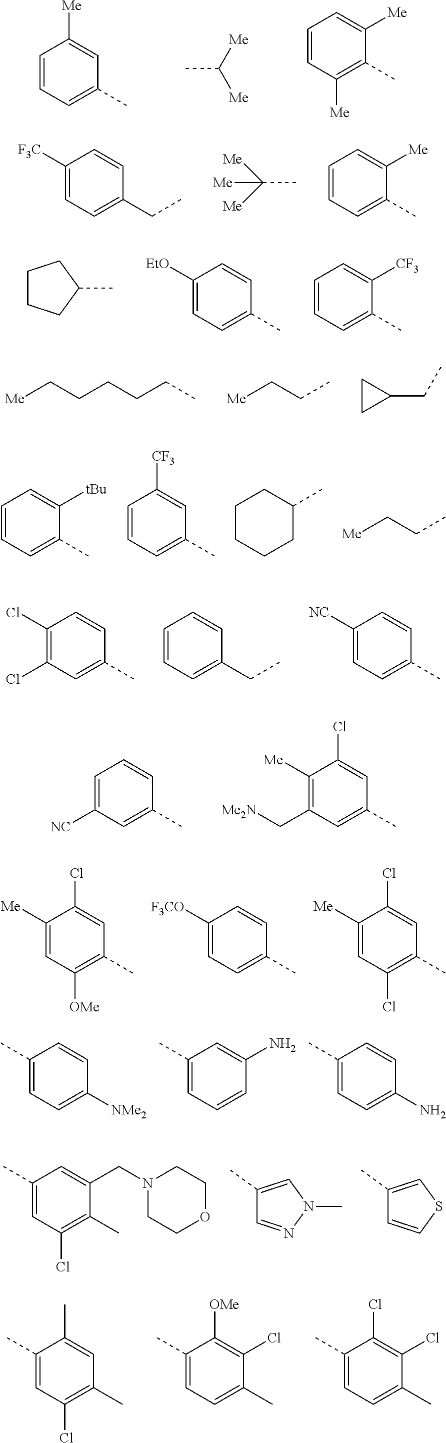

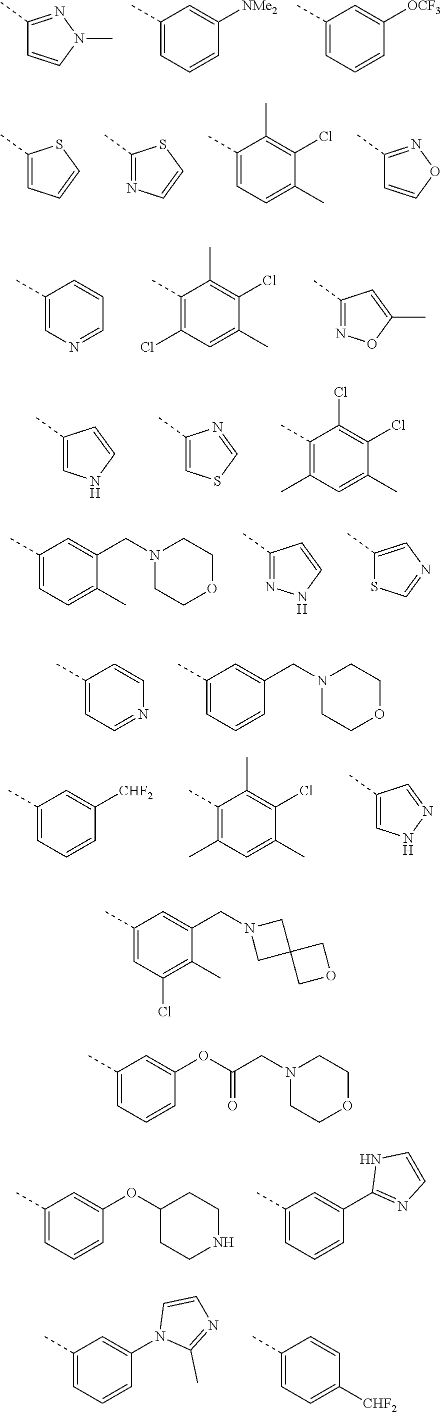

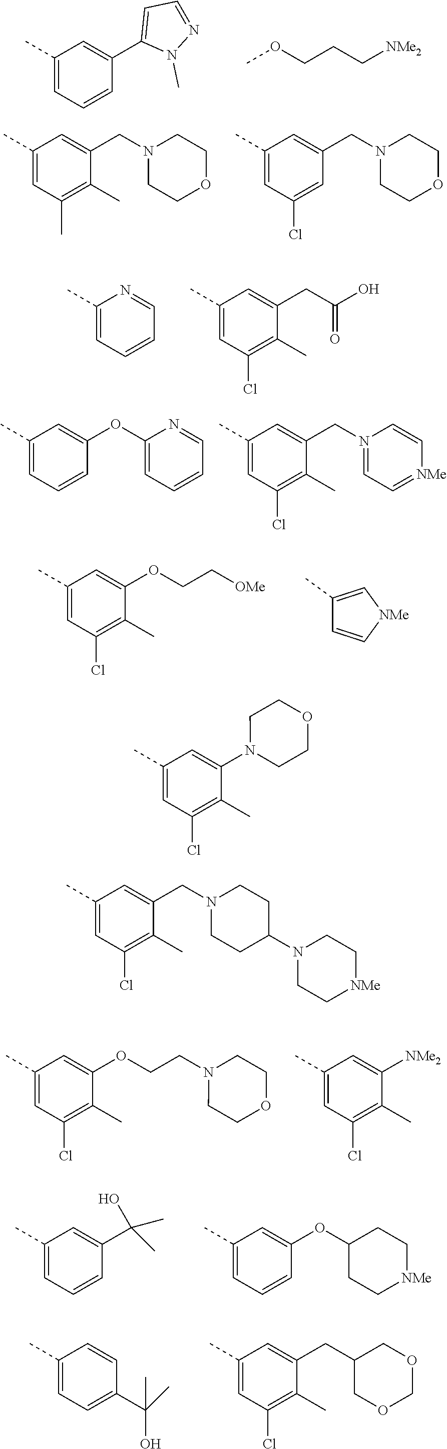

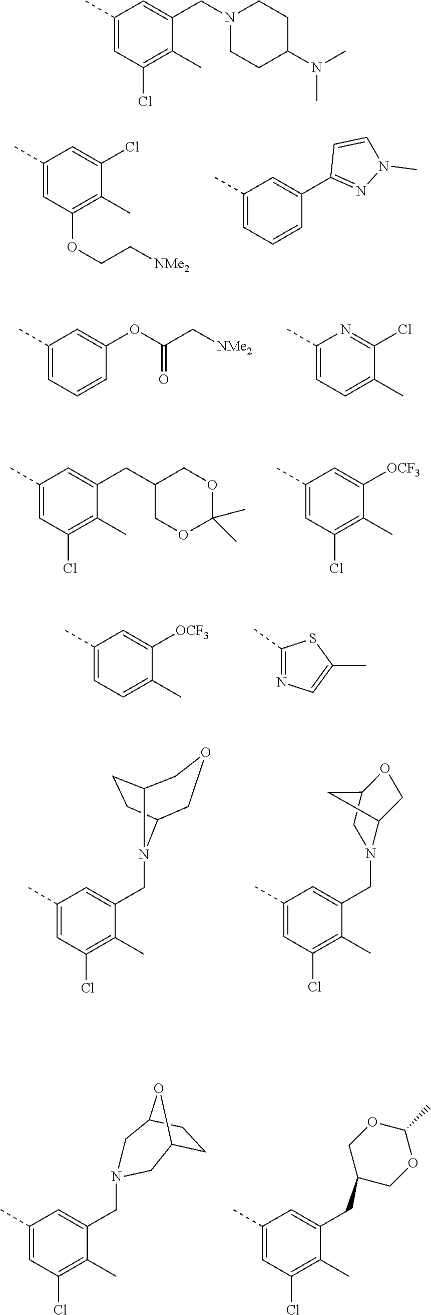

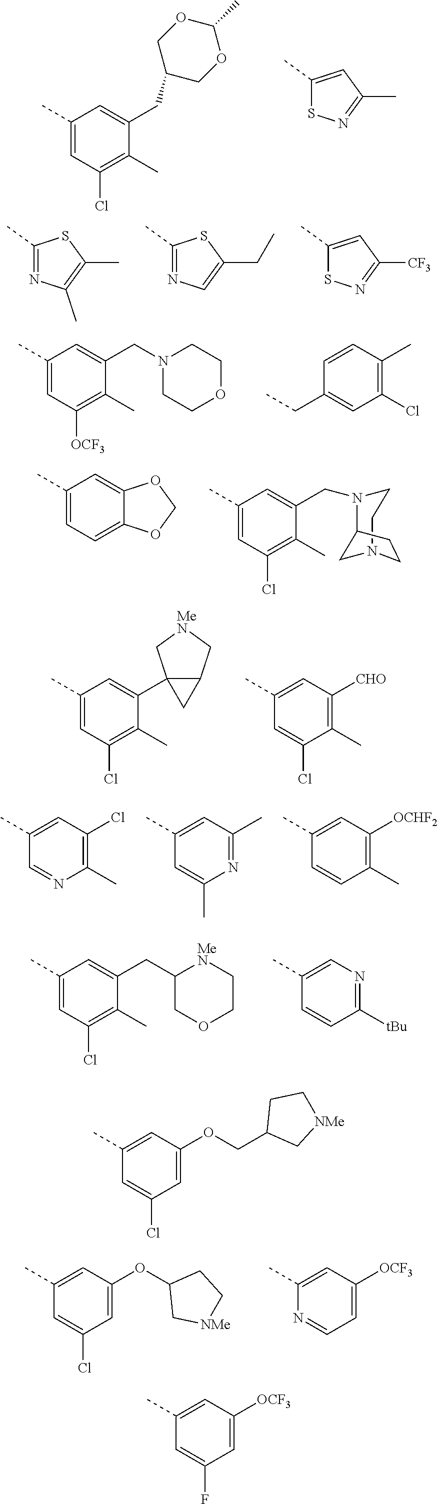

In additional embodiments: the GSPT1 modulator is a compound or a pharmaceutically acceptable salt or stereoisomer thereof of formula VI

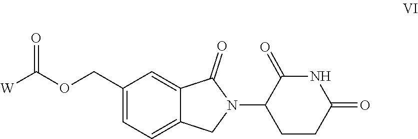

wherein W is selected from

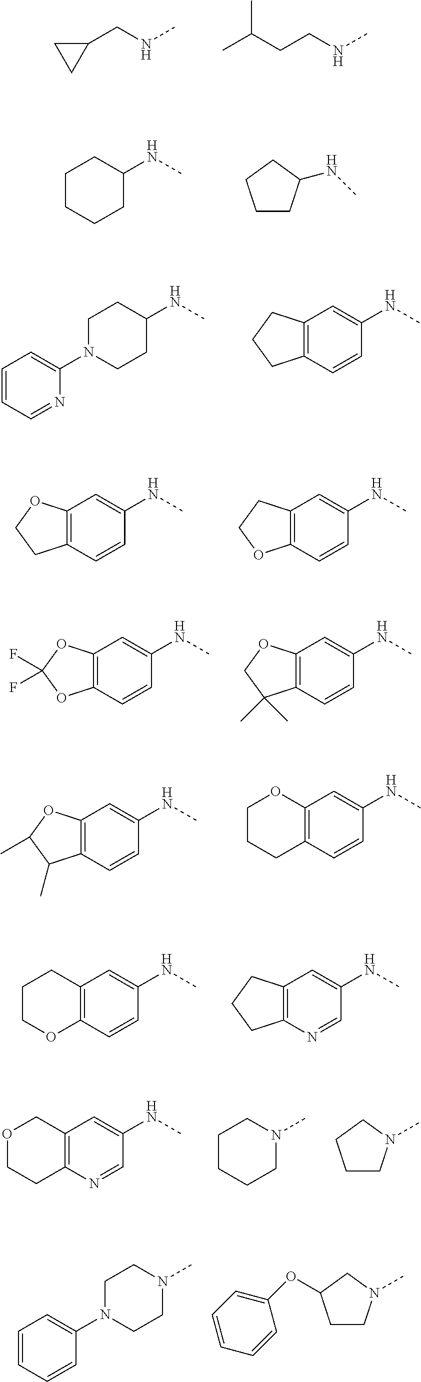

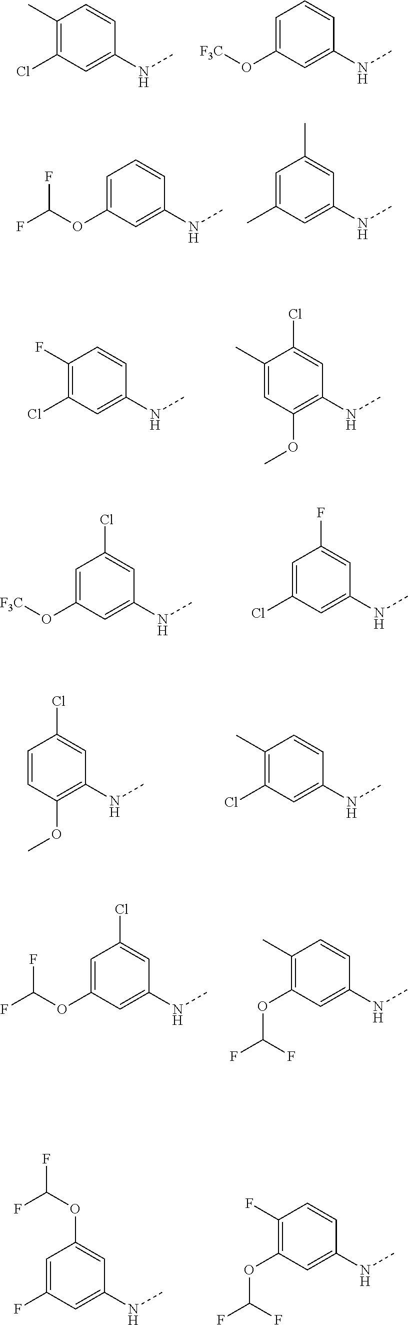

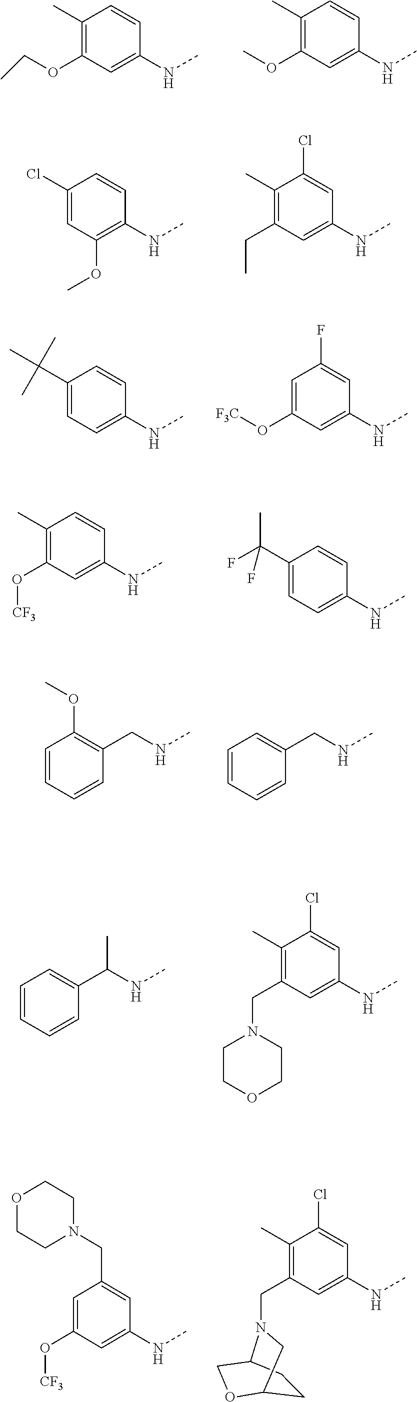

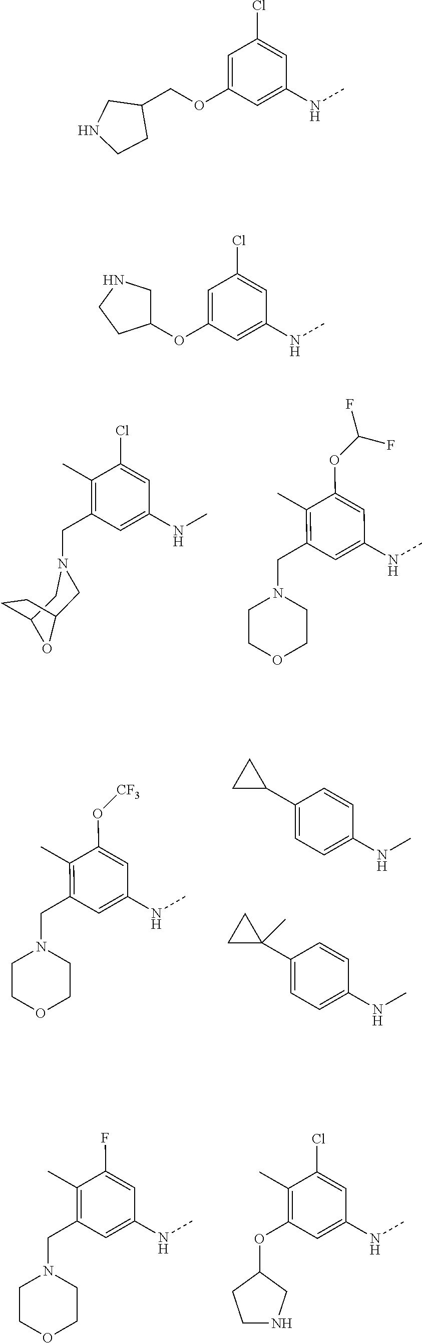

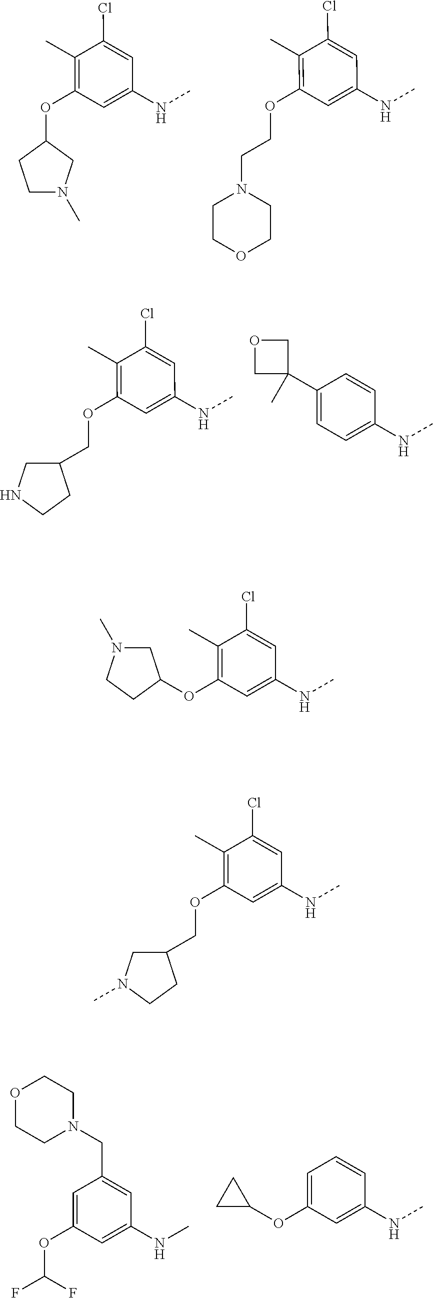

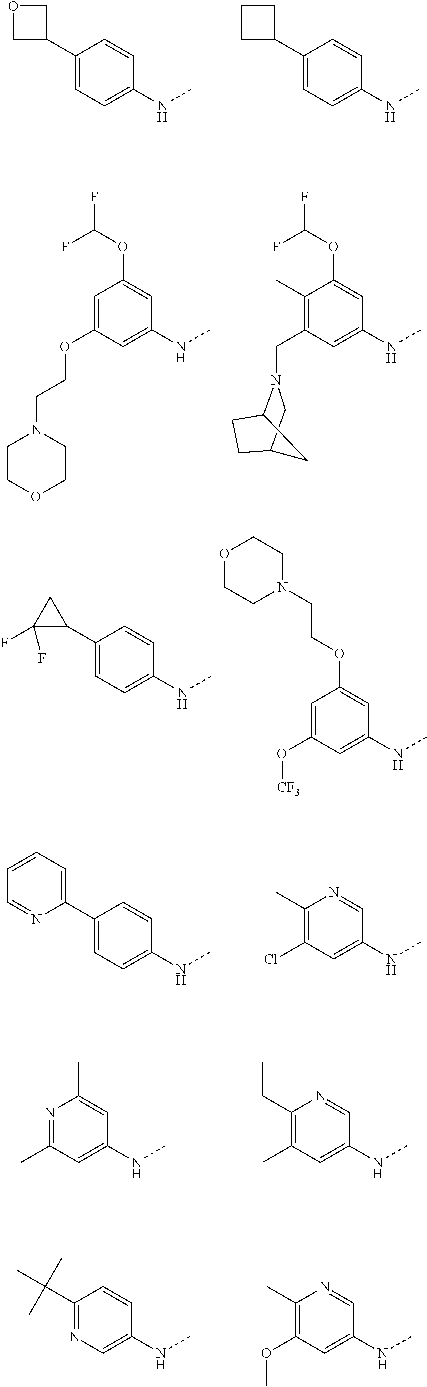

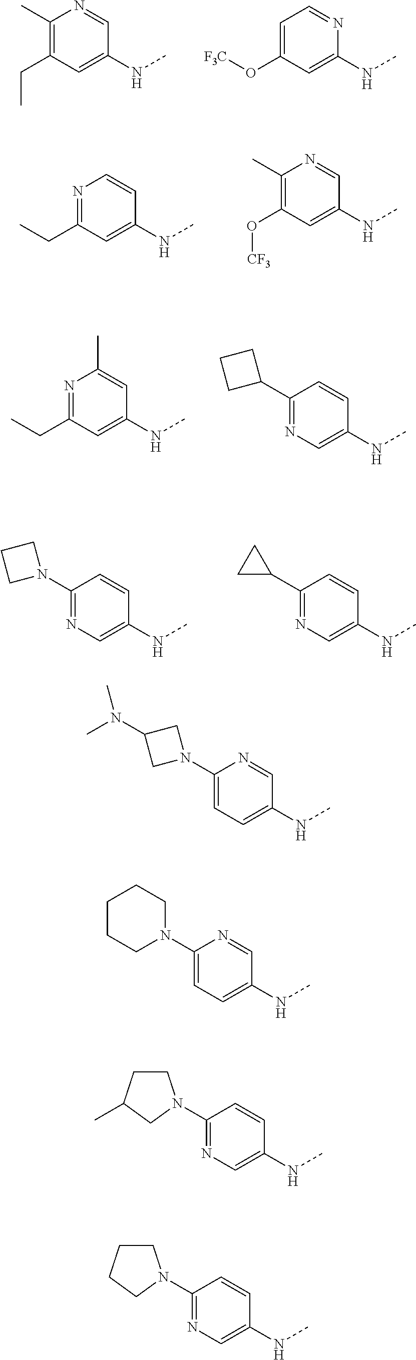

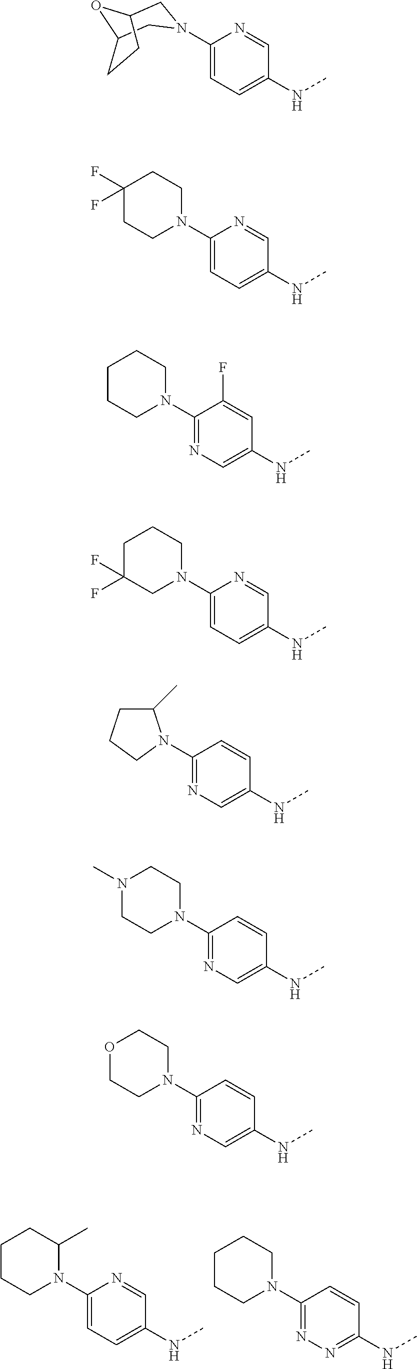

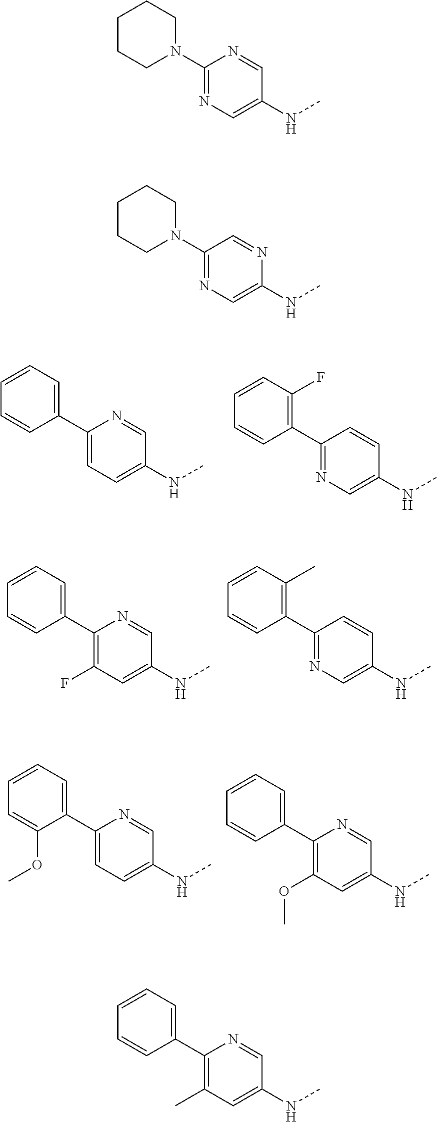

In further embodiments: the GSPT1 modulator is a compound or a pharmaceutically acceptable salt or stereoisomer thereof of formula VII or VIIa, VIIb, VIIc

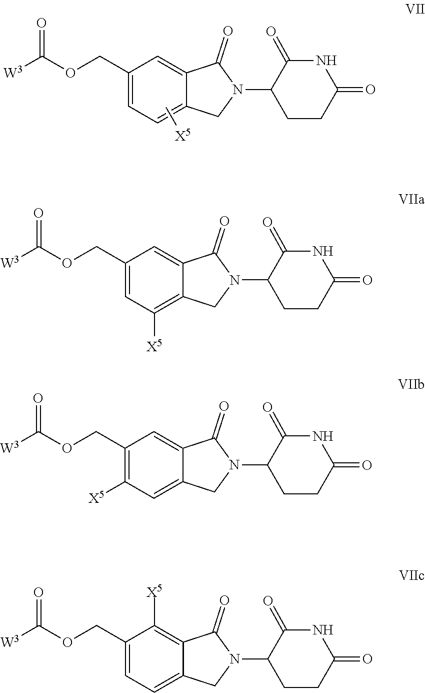

wherein X⁵ is linear or branched C₁₋₆ alkyl, —C₁₋₄ alkoxy, —CN, halogen, CF₃, CHF₂, CMeF₂, OCF₃, OCHF₂, in particular C₁₋₄ alkyl, such as methyl, —C₁₋₄ alkoxy, such as —OMe, —CN, halogen, such as F, Cl, Br; and W³ is selected from

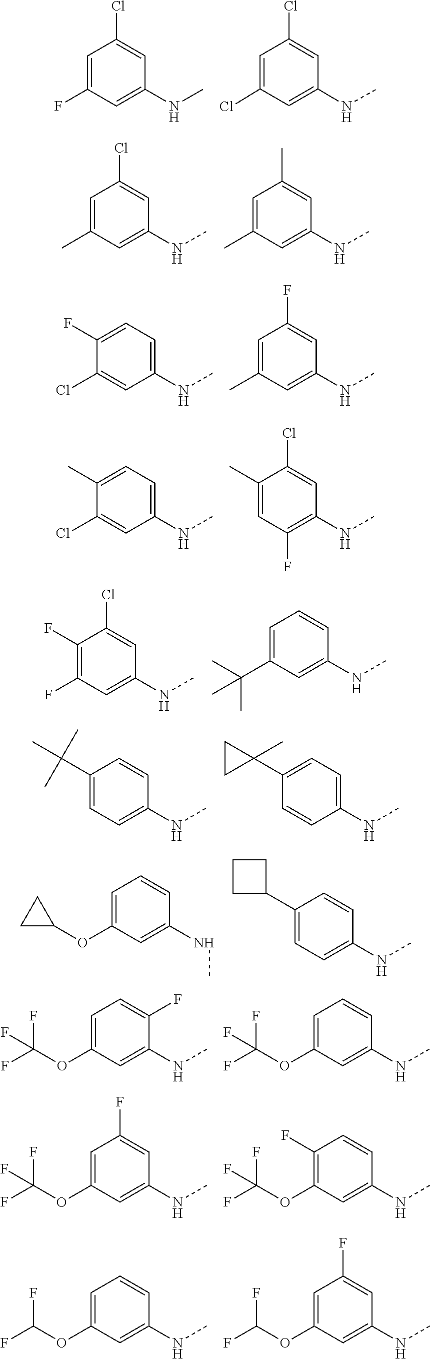

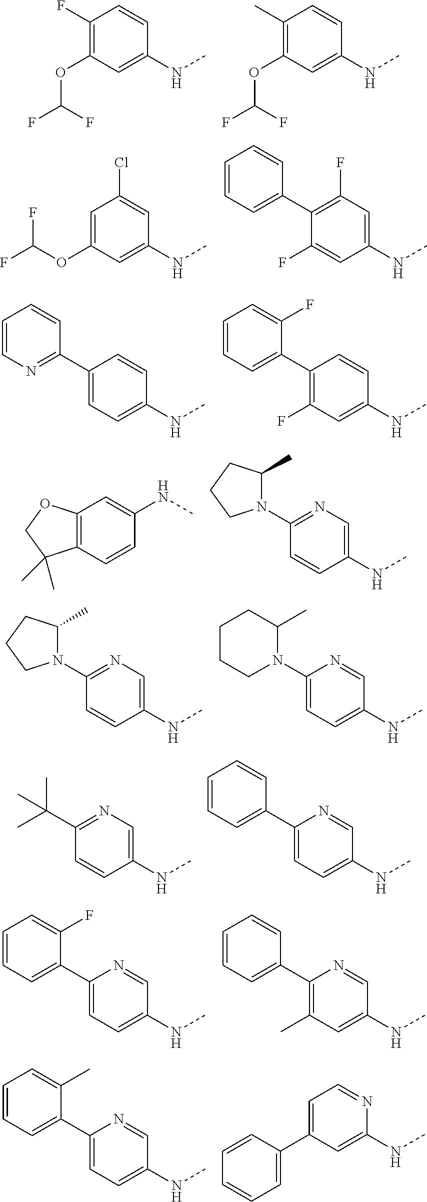

### BRIEF DESCRIPTION OF THE FIGURES

FIG. 1: Profiling of compound 8 in a representative panel of cell lines of cancers (as indicated). Viability was measured by CellTiter Glo 72 hr post treatment. Each dot represents a cancer line. From left to right the panels represent cell lines from the following cancers: TNBC: triple negative breast cancer; ER+: estrogen receptor-positive breast cancer; HER2+: human epidermal growth factor receptor 2 positive breast cancer; SCLC: small cell lung cancer; stomach, colorectal, U. bladder: urinary track (bladder); brain; pancreatic; prostate; NSCLC squam: non small cell lung cancer (squamous); NSCLC adeno: non small cell lung cancer (adenocarcinoma); ovarian; MM: multiple myeloma; AML: acute myeloid leukemia. Horizontal black lines (for each cancer sub-types) represent the mean EC50 value. The vertical axis represents EC50 (μM).

FIG. 2: Pearson correlation coefficients for compound 8 sensitivity vs mRNA expression (x-axis) and compound 8 sensitivity vs protein expression (y-axis) are plotted using a scatter plot allowing identification of genes with outlier correlation with compound 8 sensitivity. Top scoring hits are illustrated including EIF4EBP1 and EIF4EBP2. Analysis was performed based on the viability data from FIG. 1.

FIG. 3A: Representation of an unsupervised hierarchical clustering analysis for each indicated breast cancer line based on the mRNA expression (line 3), protein (line 4) and phospho protein (lines 1, 2, 5) data from the CCLE RNAseq and reverse-phase protein arrays (RPPA) datasets for this gene. Line A represents compound 8 (EC50 as shown on FIG. 1). Cluster A indicates a high EIF4EBP1 signature and sensitivity for Compound 8, cluster B indicates a low EIF4EBP1 signature and resistance to compound 8.

FIG. 3B: Representation of an unsupervised hierarchical clustering analysis for each indicated SCLC cancer line based on the mRNA expression (line 1), protein (line 2), copy number (lane 3) and phospho protein (lines 4, 5) data from the CCLE RNAseq and Reverse-phase protein arrays (RPPA) datasets for this gene. Line A represents compound 8 (EC50 as shown on FIG. 1). Cluster A indicates a high EIF4EBP1 signature and sensitivity for Compound 8, cluster B indicates a low EIF4EBP1 signature and resistance for Compound 8.

FIG. 4A; Representative examples of association between EIF4EBP1 (4EBP1) levels and sensitivity to Compound 8. Cancer subtypes are from left to right: First row: all cell lines; stomach; liver; urinary tract. Second row: breast; prostate; lung NSC (non small cell lung cancer); central nervous system. Vertical axis: compound 8 log₁₀EC₅₀. Data shows examples typically using 4EBP1 expression (“4EBP1 mRNA expression”), protein level (based on either RPPA, reverse phased protein array; “4EBP1 protein (RPPA)” or deep proteomics “4EBP1 protein”) or phosphorylated protein (for instance at tyrosine 70; “4EBP1_pT70”) as indicated. Analysis was performed based on the viability data from FIG. 1. Numbers (N) in parentheses represent the number of cell lines tested for each cancer subtypes. P represents the statistical p-value between the two groups.

FIG. 4B: Representative examples of association between EIF4EBP2 (4EBP2) levels and sensitivity to Compound 8. Cancer subtypes are from left to right: First row: all cell lines; ovary; lung NSC (non small cell lung cancer); breast. Second row: colorectal; liver; prostate; lung small cell (small cell lung cancer). Vertical axis: compound 8 log₁₀EC₅₀. Data shows examples typically using 4EBP2 expression (“4EBP2 mRNA expression”) or protein level (“4EBP2 protein”) as indicated. Analysis was performed based on the viability data from FIG. 1. Numbers (N) in parentheses represent the number of cell line tested. P represents the statistical p-value between the two groups.

FIG. 4C: Representative associations between N-Myc levels and sensitivity to Compound 8. Cancer subtypes are from left to right: First row: all cell lines; AML; liver; lung NSC (non small cell lung cancer). Second row: ovary; lung small cell (small cell lung cancer); stomach. Vertical axis: compound 8 log₁₀EC₅₀. Data shows representative examples using N-Myc expression (mRNA). Analysis was performed based on the viability data from FIG. 1. Numbers (N) in parentheses represent the number of cell line tested. P represents the statistical p-value between the two groups.

FIG. 4D: Representative associations between L-Myc levels and sensitivity to GSPT1 degrader Compound 8. Cancer subtypes are from left to right: First row: all cell lines; lung small cell (small cell lung cancer); stomach; upper aerodigestive (upper aerodigestive tract). Vertical axis: compound 8 log₁₀EC₅₀. Data shows representative examples using L-Myc expression (mRNA). Analysis was performed based on the viability data from FIG. 1. Numbers (N) in parentheses represent the number of cell line tested. P represents the statistical p-value between the two groups.

FIG. 5A: Human mammary epithelial cells, or HMECs, overexpressing c-Myc in a doxycycline-inducible manner were used to evaluate the vulnerability of Myc-driven tumors to disruption of protein translation through degradation of GSPT1. After c-Myc induction the cells displayed key biomarkers of enhanced protein translation, including upregulation and phosphorylation of 4EBP1.

FIG. 5B: Depiction of the results of a study that demonstrates that compound 8 induced cell death with an EC50 of 0.64 μM in the presence of high c-Myc expression (filled circles) but did not induce cell death at the highest concentration tested of 30 μM in the absence of doxycycline-driven c-Myc expression or after doxycycline was washed out to remove c-Myc expression in cells that previously expressed c-Myc (filled squares and filled triangles, respectively). X-axis represents m concentration of compound 8; y-axis represents viability normalized to DMSO.

FIG. 5C: Depiction of the results of a study that demonstrates that compound 8 did induce death in cells with wildtype (WT) cereblon (filled squares) but did not induce death in cells for which cereblon was knocked out (filled circles), confirming cereblon-dependence of compound 8's viability effect. X-axis axis represents m concentration of compound 8; y-axis represents viability normalized to DMSO.

FIG. 6A: Depiction of the results of a study that demonstrates that NSCLC cell lines expressing high levels of N-Myc (NCI-H1155 represented as filled triangles, ABC-1 represented as filled rhombi) were highly sensitive to treatment with compound 210, when compared to the cell lines expressing low levels of N-Myc (EBC-1 represented as empty triangles, NCI-H2023 represented as empty circles). GSPT1 was degraded by compound 210 after six hours of treatment in high N-Myc NCI-H1155 and ABC-1 cells with a DC50 of 3 nM and 22 nM, respectively (x-axis represents M concentration of compound 210; y-axis represents the viability in %). In both cell lines, we observed complete degradation of GSPT1.

FIG. 6B: Depiction of the results of a study that demonstrates that compound 210 degrades GSPT1 in a concentration dependent manner (x-axis represents M concentration of compound 210; y-axis represents relative levels of GSPT1)

FIG. 7A: In vivo anti-tumor activity of compound 8 in multiple breast cancer models. Shown are representative examples of triple negative breast cancer models with high 4EBP1 marker expression levels. Tumor growth was measured in MDA-MB-468 (ATCC HTB-132) (A), MDA-MB-231 (ATCC HTB-26) (B), MDA-MB-436 (ATCC HTB-130) (C) and CAL51 (DSMZ-ACC-302) (D) as indicated. compound 8 was dosed i.p at 37 and 75 mpk for 21 continuous days and tumor size measured every three days. Vehicle (black line), compound 8 dosed daily at 37 mpk (light grey) and 75 mpk (dark grey). X-axis represents days; y-axis represents the tumor volume (mm³). A robust and dose-dependent anti-tumor activity, including regressions, was observed in all cases.

FIG. 7B: In vivo anti-tumor activity of compound 8 in the MDA-MB-213 model. Mice were dosed daily i.p. with vehicle or compound 8 at 10 and 37 mg per kilogram i.p. or 37 mg per kilogram sub-cut. Mice were dosed for 21 days or 24 days and tumor volumes measured every 3 days. X-axis represents days; y-axis represents tumor volume in mm³.

FIG. 8A: Depiction of the results of a study that demonstrates that oral administration (PO) of compound 210 in a N-Myc-driven mouse xenograft model using the human cell line NCI-H1155 led to tumor growth inhibition (with no body weight loss observed). At a dose of 1 mg/kg once daily (filled squares), tumor growth was suppressed for two weeks. At a dose of 3 mg/kg once daily (filled triangles) or 6 mg/kg dosed for five days on and nine days off (filled triangles inverted, 5 on-9 off) tumor size decreased, became undetectable by day eight and remained so until the end of the study at day 21 (x-axis represents days post treatment initiation [days]; y-axis represents tumor volume [mm³], mean±SEM; empty circle O: vehicle, filled rhombi, gemcitabine 40 mg/kg IP, Q4D×5).

FIG. 8B: Depiction of the results of a study that demonstrates complete degradation of GSPT1 in tumors of mice treated with compound 210 at all three dose levels as compared to mice treated with vehicle control (from left to right: vehicle, 1 mg/kg, 3 mg/kg, 6 mg/kg).

FIG. 8C: Depiction of the results of a study that demonstrates that oral administration of compound 210 in a N-Myc-driven mouse xenograft model using the human cell line NCI-H1770 led to tumor growth inhibition (with no body weight loss observed). At a dose of 3 mg/kg once daily (filled triangles) or 6 mg/kg dosed for five days on and nine days off (filled triangles inverted, 5 on-9 off), tumor size decreased and remained so until the end of the study (x-axis represents days post treatment initiation [days]; y-axis represents tumor volume [mm³], mean±SEM; empty circle O: vehicle, PO, QD; filled rhombi, cisplatine 6 mg/kg IP, QW×3).

FIG. 8D: Depiction of the results of a study that demonstrates that oral administration of compound 210 in a N-Myc-driven mouse xenograft model using the human cell line NCI-H526 led to tumor growth inhibition (with no body weight loss observed). At a dose of 3 mg/kg once daily (filled triangles) or 6 mg/kg dosed for five days on and nine days off (filled triangles inverted, 5 on-9 off), tumor size decreased and remained so until the end of the study (x-axis represents days post treatment initiation [days]; y-axis represents tumor volume [mm³], mean±SEM; empty circle O: vehicle, PO, QD; filled rhombi, cisplatine 6 mg/kg IP, QW×3).

FIG. 9: Depiction of the results of a study that demonstrates robust and dose-dependent degradation of GSPT1 levels in tumors. Representative examples below show the levels of GSPT1 in MDA-MB-468 (ATCC HTB-132), MDA-MB-231 (ATCC HTB-26), MDA-MB-436 (ATCC HTB-130) and CAL51 (DSMZ-ACC-302) tumors (in lanes from top down as indicated) following three consecutive doses of compound 8 at 37 (right panel) and 75 mpk (middle panel) or administration of vehicle only (left panel). GADPH levels as control is shown in the lowest lane. Tumors were harvested 24 hr post third dose and levels of GSPT1 were determined by western blotting as indicated. Tumors were collected from the in vivo efficacy studies described in the previous FIG. 7A.

FIGS. 10A and 10B: Induced-degradation of GSPT1 following treatment with Compound 345 in NSCLC cancer cell lines. NCI-H1155 and ABC-1 were taken as representative of N-Myc positive NSCLC. NCI-H2023 and NCI-H441 as of N-Myc low representatives. 10A: western blotting analysis performed after 6 (NCI-H1155 and ABC-1) or 24 hr (NCI-H2023 and NCI-H441) posttreatment. Bortezomib addition (0.2 μM) rescued GSPT1 from degradation. 10B: corresponding densitometry analyses of GSPT1 normalized to GAPDH. DC₅₀ and Dₘₐₓ as indicated.

FIG. 11A: Anti-proliferative activity of Compound 345 against a panel of NSCLC cancer cell lines and association with N-Myc and p4EBP1 levels. Each dot represents an individual cell line. Viability activity as assessed by Cell Titer Glo assay after 72 hr.

FIG. 11B: Induced-degradation of GSPT1 following treatment with Compound 345 and anti-proliferative activity in NSCLC cancer cell lines. NCI-H1155 and ABC-1 were taken as representative of N-Myc high NSCLC. NCI-H2023 and NCI-H441 as of N-Myc low representatives. Left: densitometry analysis of GSPT1 normalized to GAPDH from western blotting following treatment with Compound 345 (as indicated). Right: 72 hr viability assay following treatment with Compound 345 as assessed by Cell Titer Glo. For both the GSPT1 expression plot and the cell viability plot, two Myc-driven cancer cell lines are represented by upright triangles and diamonds. Two non-Myc-driven cancer cell lines are represented by upside down triangles and circles.

FIGS. 12A and 12B: Timecourse experiment following the degradation of GSPT1 and concomitant downregulation of N-Myc total protein levels in NSCLC cancer lines following treatment with Compound 345. NCI-H1155 and NCI-H2023 were taken as representative of N-Myc high and low NSCLC. 12A: western blot analysis probing for GSPT1 and N-Myc following treatment with Compound 345 (as indicated). Tubulin was used as a loading control. N-Myc could not be detected by western blotting in the NCI-H2023 line. 12B: Single-sample gene set enrichment analysis (ssGSEA) results for a Myc target gene set at 6 hours (left) and 24 hours (right) after treatment. ssGSEA scores were normalized to DMSOs for each cell line and averaged for three replicates. Error bars denote+/−1 s.d. across replicates.

FIGS. 13A and 13B: In vivo anti-tumor activity of Compound 345 in the N-Myc high NCI-H1155 NSCLC subcutaneous xenograft model. 13A: Mice were dosed daily PO for 17 continuous days with vehicle or Compound 345 at 1, 3, 10 mg/kg (as indicated). Tumor volumes were measured twice weekly. X-axis represents days; y-axis represents mean tumor volume (in mm³). 13B: Compound 345 plasma concentration and GSPT1 and N-Myc total protein levels in the NCI-H1155 tumors following 5 consecutive daily doses of Compound 345 at 1 and 10 mg/kg (timepoints as indicated). Data represents the mean±SEM.

FIGS. 14A and 14B: In vivo mouse patient-derived xenograft experiment. Compound 345 treatment significantly prolonged survival in biomarker positive (high N-Myc or L-Myc mRNA expression) patient-derived xenografts (14A)—but not in the biomarker negative patient-derived xenografts (14B). p values determined by log-rank (Mantle-Cox) test.

FIG. 15A: Anti-proliferative activity of Compound 345 against a panel of SCLC cancer cell lines and association with L-Myc levels. Each dot represents an individual cell line. Viability activity as assessed by Cell Titer Glo assay after 72 hr.

FIG. 15B: Timecourse experiment following the degradation of GSPT1 and concomitant downregulation of L-Myc in SCLC cancer lines following treatment with Compound 345. NCI-H1836 and NCI-H1876 were taken as two representatives of L-Myc high SCLC. Western blot analysis probing for GSPT1 and L-Myc following treatment with Compound 345 as indicated. GAPDH was used as a loading control.

FIGS. 16A, 16B and 16C: Activity of Compound 345 in the c-Myc high multiple myeloma MM1S cell line. 16A: Degradation of GSPT1 and concomitant downregulation of c-Myc following treatment with Compound 345 as indicated (24 hr post treatment). GAPDH was used as a loading control. 16B: Apoptosis induction in the MM1S cell line following treatment with Compound 345 for 48 hr as assessed by Caspase 3/7 apoptosis Glo assay. Lenalidomide was used as a benchmark. 16C: Antiproliferative activity of Compound 345 (as indicated) following treatment with Compound 345 for 72 hr as assessed by Cell Titer Glo assay.

FIG. 17: In vivo anti-tumor activity of Compound 345 in the c-Myc high MM1S multiple myeloma subcutaneous xenograft model. Mice were dosed daily PO with vehicle or Compound 345 at 1, 3, 10 mg/kg (as indicated). Tumor volumes were measured twice weekly. X-axis represents the number of days for treatment duration; y-axis represents mean tumor volume (in mm³)±SEM. Lenalidomide was used as a positive control for this model. Drug-free vehicle is represented by black circles; lenalidomide 50 mg/kg represented by gray circles; Compound 345 1 mg/kg represented by triangles; Compound 345 3 mg/kg represented by upside down triangles; Compound 345 10 mg/kg represented by diamonds.

FIGS. 18A, 18B and 18C: Activity of Compound 345 in the c-Myc high WSU-DLCL2 lymphoma cell line. 18A: Degradation of GSPT1 and concomitant downregulation of c-Myc following treatment with Compound 345 as indicate (24 hr post treatment)d. GAPDH was used as a loading control. 18B: Antiproliferative activity of Compound 345 (as indicated) following treatment with Compound 345 for 72 hr as assessed by Cell Titer Glo assay. 18C: In vivo anti-tumor activity of Compound 345 in the WSU-DCLC2 subcutaneous xenograft model. Mice were dosed daily PO with vehicle or Compound 345 at 3, 10 mg/kg (as indicated). Tumor volumes were measured twice weekly. X-axis represents the number of days for treatment duration; y-axis represents mean tumor volume (in mm³)±SEM. CHOP was used as a positive control for this model. Drug-free vehicle is represented by black circles; CHOP combination represented by light squares; Compound 345 3 mg/kg represented by triangles; Compound 345 10 mg/kg represented by dark squares.

FIGS. 19A, 19B, and 19C: Activity of Compound 345 in the c-Myc high DOHH-2 lymphoma cell line. 19A: Degradation of GSPT1 and concomitant downregulation of c-Myc following treatment with Compound 345 as indicated (24 hr post treatment). GAPDH was used as a loading control. 19B: Antiproliferative activity of Compound 345 (as indicated) following treatment with Compound 345 for 72 hr as assessed by Cell Titer Glo assay. 19C: In vivo anti-tumor activity of Compound 345 in the DOHH-2 subcutaneous xenograft model. Mice were dosed daily PO with vehicle or Compound 345 at 3, 10 mg/kg (as indicated). Tumor volumes were measured twice weekly. X-axis represents the number of days for treatment duration; y-axis represents mean tumor volume (in mm³)±SEM. CHOP was used as a positive control for this model.

FIG. 20A: Anti-proliferative activity of Compound 345 against a panel of multiple myeloma:cancer cell lines. Each dot represents an individual cell line. Viability activity as assessed by Cell Titer Glo assay after 72 hr.

FIG. 20B: Degradation of GSPT1 and concomitant downregulation of c-Myc following treatment with Compound 345 in the MM1S multiple myeloma cancer cell line. Western blot analysis performed 24 hr post treatment initiation. GAPDH was used as a loading control. GSPT1 DC₅₀ and Dₘₐₓ as indicated.

FIG. 21A: Anti-proliferative activity of Compound 345 against a panel of lymphoma cancer cell lines. Each dot represents an individual cell line. Viability activity as assessed by Cell Titer Glo assay after 72 hr.

FIGS. 21B and 21C: Degradation of GSPT1 and concomitant downregulation of c-Myc following treatment with Compound 345 in the WSU-DLCL2 (21A) and DOHH-2 (21C) lymphoma cancer cell lines. Western blot analysis performed 24 hr post treatment initiation. GAPDH was used as a loading control. GSPT1 DC₅₀ and Dₘₐₓ as indicated.

### DETAILED DESCRIPTION

The present disclosure relates to new methods that are useful in the prediction of the responsiveness of a cancer patient to a treatment with one or more GSPT1 negative modulators. These methods include determining the level of one or more biomarkers, in particular myc transcription factor markers or surrogate markers thereof, such as translation addicted markers as defined herein. In some embodiments these biomarkers include, but are not limited to L-Myc, N-Myc, c-Myc, EIF4EBP1 and/or EIF4EBP2. The present disclosure also relates to applications of these methods, which includes stratifying malignancies, in particular myc-driven cancers, and thereby devising optimized and personalized therapies for these cancer patients, as well as optimizing the selection of patient populations for respective clinical trials.

In some embodiments the present disclosure relates to methods to predict and/or monitor the responsiveness of a myc-driven cancer patient to treatment with a GSPT1 negative modulator.

In some embodiments the present disclosure relates to methods to predict and/or monitor the effectiveness of a GSPT1 negative modulator in the treatment of a myc-driven cancer or tumor. In some embodiments the present disclosure relates to methods to assess and monitor the progress of a treatment of a myc-driven cancer with a GSPT1 negative modulator.

In some embodiments the myc-driven cancer or tumor as defined herein refers in particular to breast cancer, SCLC, NSCLC, colorectal cancer, stomach cancer, pancreatic cancer, gastric cancer, liver cancer, prostate cancer, multiple hematological cancers (e.g. AML), neuroblastoma, neuroendocrine cancer, e.g., neuroendocrine prostate cancer (for example, NEPC (castration-resistant neuroendocrine prostate cancer)) and lung neuroendocrine tumors (Lu-NETs), liver cancer.

In some embodiments the myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM). In some embodiments the myc-driven cancer or tumor as defined herein refers in particular to lymphoma, AML and MM.

Unless stated otherwise the following definitions apply throughout the text:

It is understood that the indication “a” or “the” is not limited to the singular form but also extends to the plural form, e.g. referring to a cell (such as a cancer cell) also includes cells.

The meaning of the term “cancer” or “cancerous” as used herein refers to a physiological condition characterized by cellular hyperproliferation, more particularly pathological hyperproliferation. The term cancer includes a cancer cell (or cancer cell derived therefrom) or a tumor or tumor cell and corresponds to all stages of the disease (precancerous as defined below, early, moderately advanced and advanced).

The term “subject” refers to a mammal, including, but not limited to, humans, primates, animals, rodents, preferably a human. A subject includes both a subject suffering from a cancer, e.g. a myc-driven cancer, as defined herein (also referred to as a patient), as well as a healthy subject. The term “patient” or “cancer patient” as used herein refers to subject that has been diagnosed with cancer, in particular a myc-addicted or a myc-driven cancer as defined herein. A patient also refers to a subject in a precancerous condition, i.e. which is a condition or lesion involving abnormal cells which are associated with an increased risk of developing into cancer, a subject who is afflicted with cancer and is suspected to have a metastatic spread of the primary tumor, a subject who was previously afflicted with cancer and is now in remission, and a subject who was previously afflicted with cancer and is now in remission (and thus has an increased risk for reoccurrence of the cancer, i.e., as a means to monitor for a reoccurrence of the cancer). A patient that is responsive to a specific treatment is also referred to as a responder, while a patient that is non-responsive to a specific treatment is also referred to as a non-responder

The term “myc transcription factor” refers to the myc family of transcription factors, which includes N-Myc (MYCN proto-oncogene; UniProtKB P04198 (MYCN_HUMAN); GenBank Gene ID 4613), L-Myc (MYCL proto-oncogene; UniProtKB P12524 (MYCL_HUMAN); GenBank Gene ID 4610) and c-Myc (MYCN proto-oncogene; UniProtKB P01106 (MYC_HUMAN); GeneBank Gene ID 4609). Myc is a member of a family of regulator genes and proto-oncogenes that code for transcription factors, namely the myc transcription factors. Myc leads to the increased expression of many genes, some of which are involved in metabolic reprogramming and cell proliferation, contributing to the formation of cancer. Unless specified otherwise (or apparent from the context), the term “myc” used herein includes the myc gene, the mRNA of the myc gene, and the myc transcription factor (also referred to as myc factor or myc protein). The level of a myc transcription factor may be determined directly, as well as indirectly by determining for example the level of its mRNA, the copy number of the myc gene encoding the myc protein, the level of some posttranslationally modified product of the myc protein, a metabolite of a myc protein or any other form that may be a representative measure for presence and/or level of a myc protein.

The term “EIF4EBP1” and “EIF4EBP2” (or 4EBP1 and 4EBP2), as used herein, refer to the human eukaryotic translation initiation factor 4E-binding proteins 1 and 2, respectively. EIF4EBP1 and EIF4EBP2 interact with the eukaryotic translation initiation factor 4E (eIF4E) by preventing its assembly into the eIF4F complex. The phosphorylated forms of EIF4EBP1 and EIF4EBP2 are regulated by mTOR, in the context of the mTORC1 complex, and activate translation. Nucleic acid and amino acid sequences for EIF4EBP1 (UniProtKB Q13541 (4EBP1_HUMAN) GenBank Gene ID 1978) and EIF4EBP2 (UniProtKB Q135442 (4EBP2_HUMAN) GeneBank Gene ID 1979) are known in the art and are publicly available in the GenBank database maintained by the U.S. National Center for Biotechnology Information.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-Myc and/or L-Myc and/or c-Myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell). An “overexpression” is a significantly higher level of a biomarker and refers to a level in a test sample that is greater than the standard error of the assay employed to assess the level. In some embodiments, the level is at least 10%, such as 10, 15, 20% or more higher than the expression activity or level of the biomarker in a control sample (as defined herein, e.g, a sample from a healthy subject not afflicted with the cancer associated with the biomarker or a sample from healthy tissue from the same patient). In some embodiments, the average level of the biomarker in several control samples. An “underexpression” is a significantly lower level of a biomarker and refers to a level in a test sample that is at least 10%, such as 10, 15, 20% or more lower than the level of the biomarker in a control sample (e.g., sample from a healthy subject not having the biomarker associated disease).

Thus, a myc-driven cancer cell may refer to a cell that has an increase (1.5×, 2×, 3×, 4×, etc.) in the number of copies (e.g., 6 copies or more (e.g., 7, 8, 9 or 10) of one of the myc family members, or a surrogate marker thereof.

In some embodiments, a myc-driven cancer includes, but is not limited to breast cancer (e.g. basal-like breast cancer) and breast invasive carcinoma, lung carcinoma (SCLC and NSCLC), colorectal cancer, stomach cancer, uterine cancer, ovarian cancer, lymphoma, pancreatic cancer, neuroblastoma, neuroendocrine cancer, e.g., neuroendocrine prostate cancer (for example, NEPC (castration-resistant neuroendocrine prostate cancer)) and lung neuroendocrine tumors (Lu-NETs), gastric cancer, liver cancer, hematological cancers, Burkitts' Lymphoma and others, and may have or may have not undergone any treatment. Myc-driven cancers include solid cancers and blood borne (or liquid) cancers.

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

The term “GSPT1” (G1 To S Phase Transition Protein 1 Homolog) refers to polypeptides (i.e. polypeptides, peptides, proteins) comprising the amino acid sequence of any GSPT1, such as a human GSPT1 protein (e.g., human GSPT1 isoform 1, GenBank Accession No. NP_002085.3; or human CRBN isoform 2, GenBank Accession No. NP 001123478.2 and others), and related polypeptides, including SNP variants thereof. Related GSPT1 polypeptides include allelic variants (e.g., SNP variants), splice variants, fragments, derivatives, substitution variant, deletion variant, insertion variant, fusion polypeptides, and interspecies homologs, which, in certain embodiments, retain GSPT1 activity and/or are sufficient to generate an anti-GSPT1 immune response. GSPT1 is a translation termination factor, which is involved in translation termination in response to the stop termination codons UAA, UAG, and UGA, and facilitates release of a nascent peptide from the ribosome. In addition, GSPT1 is also involved in several other critical cellular processes, such as cell cycle regulation, cytoskeleton organization and apoptosis. GSPT1 stimulates the activity of eRF1 and is a component of the transient SURF complex, which recruits UPF1 to stalled ribosomes in the context of nonsense-mediated decay (NMD) of mRNAs. GSPT1 has been implicated as an oncogenic driver of several different cancer types, including breast cancer (Wang, Shuyang et al, Breast Cancer Res Treat. 2018, 171, 199-207), lung cancer, leukemia, hepatocellular carcinoma, gastric cancer (Tian, Q-G et al, Eur Rev Med Pharmacol Sci. 2018, 22, 4138-4145), and prostate cancer. See, e.g., Brito, et al., Carcinogenesis, 2005, 26, 2046-49; Brito, et al., Cane. Genet. Cyto., 2009, 195, 132-42; Tavassoli, et al., Med. Oncol., 2011, 29, 1581-85; Wright and Lange, Rev. Urol., 2007, 9, 207-213; Hoshino, et al., Apoptosis, 2012, 17, 1287-99; Liu, et. al., PLOS One, 2014, 9, e8637; Jean-Jean, et al., Mol. Cell. Bio., 2007, 27, 5619-29. GSPT1 may also contribute to glial scar formation and astrogliosis after a central nervous system (CNS) injury (e.g., Ishii et al., J. Biol. Chem., 2017, 292, 1240-50.

The term “modulating” or “modulate” generally means interacting or interact with a target either directly or indirectly so as to alter the activity of the target, either reducing or inhibiting the expression and/or activity of, or alternatively increasing the expression and/activity of, a target molecule, e.g., GSPT1, e.g., as measured using a suitable in vitro, cellular, or in vivo assay (which will usually depend on the target involved), by at least 5%, at least 10%, at least 25%, at least 50%, at least 60%, at least 70%, at least 80%, at least 90%, or more, inclusive, compared to activity of the target in the same assay under the same conditions but without the presence of an agent. The skilled person knows that “modulating” involves a change in activity or function of the target, for example by effecting a change in affinity, avidity, specificity, selectivity, sensitivity of a target molecule, such as GSPT1, for one or more of its ligands, receptors, and/or binding partners.

Thus, as used herein, the term “modulator” and in particular “GSPT1 modulator” refers to any (modulatory) entity or combination of entities that interact with GSPT1 either directly or indirectly and thereby modulate, i.e. change, alter or modify, at least to some extent, the expression, function, activity and/or stability of GSPT1 with measurable affinity. In some embodiments such (modulatory) entities may be selected from a polypeptide, peptide, glycoprotein, a peptidomimetic, an antigen binding protein (e.g. an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a (hetero) bifunctional compound such as a PROTAC or HyT molecule, or a genetic construct for targeted gene editing (e.g. a CRISPR/Cas9 construct, a guide nucleic acid (gRNA or gDNA), a tracrRNA or the like).

GSPT1 modulators that decrease expression, function, activity and/or stability of GSPT1 are referred to as “GSPT1 negative modulators” and may be referred to as inhibitors, antagonists or degraders. Preferred GSPT1 negative modulators cause degradation of GSPT1. Such negative modulators are referred to a targeted protein degraders (TPD) and include GSP1 molecular glue degraders and PROTACs. GSPT1 TPDs act by bringing GSPT1 into proximity with cereblon, leading to ubiquination and subsequent degradation of GSPT1.

A decrease refers to a statistically significant decrease. A decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100%.

In some embodiments, a GSPT1 modulator is a direct modulator, meaning that it interacts directly with either GSPT1 or a nucleic acid encoding GSPT1. In some embodiments, a modulator is an inverse agonist, antagonist, a (binding) inhibitor, and/or a degrader. In some embodiments, a GSPT1 modulator is a GSPT1 inhibitor, which is capable of binding and inhibiting or decreasing the functional activity of GSPT1 in vivo and/or in vitro with measurable affinity. In some embodiments, the GSPT1 inhibitor may inhibit GSPT1 expression by at least about 10%, at least about 30%, at least about 50%, at least about 70, 75 or 80%, still by 85, 90, 95, or 100%. In some embodiments, an inhibitor has an IC₅₀ and/or binding constant of less than about 50 μM, less than about 1 μM, less than about 500 nM, less than about 100 nM, less than about 10 nM, or less than about 1 nM. In some embodiments, a GSPT1 negative modulator is a GSPT1 degrader, which is capable of degrading the functional activity of GSPT1 in vivo and/or in vitro. In some embodiments, a GSPT1 degrader is binding to both GSPT1 and an E3 ligase with measurable affinity resulting in the ubiqitination and subsequent degradation of GSPT1. In some embodiments, a degrader has an DC₅₀ of less than about 50 μM, less than about 1 μM, less than about 500 nM, less than about 100 nM, less than about 10 nM, or less than about 1 nM. All of the effects of a modulator can be determined in any suitable manner and/or using any suitable assay known in the art.

The term “(biological) marker” or “biomarker” or “stratification marker” used in the context of the present disclosure refers to a measurable entity whose detection indicates a particular biological state, such as, for example, the presence of cancer. In some embodiments, biomarkers can be determined individually. In some embodiments, several biomarkers can be measured simultaneously. A biomarker may be any entity, such as a mRNA, DNA, a polypeptide, a protein including posttranslationally modified forms, such as phosphorylated forms (e.g. mono- or biphosphorylated forms), metabolites and the like, which may be differentially present in a sample taken from a cancer patient (i.e. which may be present at an elevated or decreased level in a sample of a cancer patient) as compared to a control or reference sample as defined herein. Determination of the presence, absence and specific level of a biomarker is carried out by appropriate quantification methods as disclosed herein and known in the art, i.e. by direct measurement of the biomarker, by indirect quantification of the gene expression of the encoding gene of the biomarker, for example by quantification of the expressed mRNA encoding for the respective biomarker. Thus, in some embodiments, a biomarker as used herein indicates a change in the level of mRNA expression that may correlate with the risk or progression of a cancer, or with the susceptibility of cancer to a given treatment. In some embodiments, the biomarker is a nucleic acid, such as mRNA or cDNA. In some embodiments, a biomarker indicates a change in the level of polypeptide or protein expression that may correlate with the risk or progression of a cancer, or patient's susceptibility to treatment. In some embodiments, the biomarker can be a polypeptide or protein, or a fragment or a postmodified, e.g. phosphorylated, form thereof. The relative level of specific proteins can be determined by methods known in the art, such as e.g. antibody based methods, such as an immunoblot, enzyme-linked immunosorbent assay (ELISA), copy number variation analysis or other methods.

A marker for use in the methods of the present disclosure is a myc transcription factor or a surrogate marker thereof such as a translation addicted marker as defined herein, and combinations thereof. The term “surrogate marker”, as used herein, refers to an entity whose presence, level, or form, may act as a proxy for presence, level, or form of another entity (e.g., a biomarker) of interest. In some embodiments, a marker for use in the present invention is one or more of N-Myc, L-Myc, c-Myc, EIF4EBP1 and/or EIF4EBP2. In some embodiments the marker for use in the present methods is EIF4EBP1 and/or EIF4EBP2 and/orc-Myc, which may act as a stratification marker for patients afflicted with cancer, for example, a myc-driven cancer, with regards to their selection for, or responsiveness to, treatment with a GSPT1 negative modulator. In some embodiments the marker for use in the present methods is EIF4EBP1 and/or EIF4EBP2 and/or L-Myc, which may act as a stratification marker for patients afflicted with a cancer, for example, a myc-driven cancer, with regards to their selection for, or responsiveness to treatment with a GSPT1 negative modulator. In some embodiments the marker for use in the present methods is N-Myc, which act as a stratification marker for patients afflicted with cancer, for example, a myc-driven cancer, with regards to their selection for, or responsiveness to treatment with a GSPT1 negative modulator. In some embodiments the marker for use in the present methods is L-Myc, which act as a stratification marker for patients afflicted with cancer, for example, a myc-driven cancer, with regards to their selection for, or responsiveness to, treatment with a GSPT1 negative modulator. In some embodiments the marker for use in the present methods is EIF4EBP1, which act as a stratification marker for patients afflicted with cancer, for example, a myc-driven cancer, with regards to their selection for, or responsiveness to, treatment with a GSPT1 negative modulator. In some embodiments the marker for use in the present methods is EIF4EBP2, which act as a stratification marker for patients afflicted with cancer, for example, a myc-driven cancer, with regards to their selection for, or responsiveness to, treatment with a GSPT1 negative modulator.

The biomarker status before, during or after therapy, may be used for assessing the likelihood of response of a cancer to a treatment of a GSPT1 negative modulator, wherein the biomarker status refers to the altered (absolute or relative) presence or absence of the biomarker in a biological sample as defined herein, in a patient or a clinical subset of patients afflicted with cancer. The present disclosure shall not be restricted to any particular method for determining the level of a given biomarker, but shall encompass all means that allow for a quantification, or estimation, of the level of said biomarker, either directly or indirectly. The level of a biomolecule used in the present methods includes therefore a parameter describing the absolute amount of a biomarker in a given sample, for example as absolute weight, volume, or molar amounts; or alternatively pertains to the relative amounts with regards to a reference value. The level of a biomarker or the biomarker status may be assessed or confirmed as disclosed herein, such as by, e.g, (1) increased or decreased copy number (e.g, by FISH (fluorescence in situ hybridization), FISH plus SKY (spectral karyotyping), SMRT (single-molecule real-time sequencing), or qPCR (quantitative PCR), overexpression or underexpression of a biomarker nucleic acid (e.g, by ISH (in situ hybridization), Northern Blot, qPCR or NGS (next generation sequencing)), increased or decreased biomarker protein level (e.g, by IHC (immunohistochemistry)), and the like.

The term “level” refers to the amount, accumulation, or rate of a biomarker molecule. A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. As used herein, the term “level” is a general term to include “expression level”, copy number or any other wording used for quantification of a biomolecule such as a gene, a nucleic acid, a protein, a metabolite, and the like. Consequently, an “altered level” refers to an increased or decreased expression level or copy number of a biomarker in a test sample, e.g., a sample derived from a cancer patient suffering from cancer, that is greater or less than the standard error of the assay employed to assess expression level or copy number, of the biomarker (or other reference marker) in a control sample The term “level” refers to an absolute amount of a molecule in a sample or a relative amount of the molecule, determined under steady-state or non-steady-state conditions.

The term “stratification” as used herein refers to the ability of predicting the responsiveness of a patient to the treatment and thus allowing to alter the treatment with regards to its continuation and/or the specific regimen. The term “stratifying” as used herein refers to the ability to sorting individuals into different classes or strata based on their responsiveness, or predicted responsiveness, to a specific treatment. For example, stratifying a patient population with myc-driven cancer involves assigning the individuals on the basis of their responsiveness, or predicted responsiveness, to the treatment with a GSPT1 negative modulator using the methods of the disclosure.

The level of a stratification marker, or marker, from a patient sample can be different when compared to the level of the stratification marker, or marker (or a different molecule suitable as control molecule) in a control sample. This change can be about 5%, 10%, 20%, 30%, 40%, 50%, 60%, 70%, 90%, 100%, 200%, 300%, 500%, 1,000%, 5,000% or more of the comparative control molecule level, which with regards to the present methods would indicate an increase or a decrease in responsiveness to the treatment.

In some embodiments, the level of a stratification marker, or marker, from a patient sample can be higher or “elevated” when compared to the level of the stratification marker, or marker (or a different molecule suitable as control molecule) in a control sample. This increase or elevated level can be about 5%, 10%, 20%, 30%, 40%, 50%, 60%, 70%, 90%, 100%, 200%, 300%, 500%, 1,000%, 5,000% or more of the comparative control molecule level, which with regards to the present methods would indicate a responsiveness or non-responsiveness to the treatment depending on the nature of the biomarker used. Alternatively, the level of a marker may be decreased by for example 99%, 95%, 90%, 80%, 70%, 60%, 50%, 40%, 30%, 20%, 10%, 1% or less of the comparative control molecule level, which with regards to the present methods would indicate a responsiveness or non-responsiveness to the treatment depending on the nature of the marker used.

The term “expression profile” (or “profile”) or “expression value” (or “value”) as used in the context of the present methods refers to the extent of expression of one of the markers of the disclosure (or a molecule other than the stratification markers used as control molecule) measured in a sample of a patient afflicted with cancer, for example, a myc-driven cancer (or in a control sample) in accordance with the methods of the present disclosure. It includes both the expression on the nucleic acid or polypeptide level, which includes modified polypeptides, that have in addition undergone posttranslational modifications such as phosphorylation.

The term “(patient) predictive profile” refers to an expression profile that has been established from a patient or patient population with known responsiveness to treatment with a GSPT1 negative modulator. A responsive predictive profile is obtained from a patient or patient population suffering from cancer, e.g. myc-driven cancer, that are responsive to treatment with a GSPT1 negative modulator. A non-responsive predictive profile is obtained from a patient or patient population suffering from cancer, e.g. myc-driven cancer, that are non-responsive to treatment with a GSPT1 negative modulator.

The term “responsiveness” or “sensitivity” and “responsive” or sensitive” when made in reference to a cancer treatment refers to the degree of effectiveness of a cancer treatment by reducing or decreasing the symptoms of the cancer being treated, which includes cessation or reduction of tumor growth or tumor recurrence, partial or full remission of tumors. Thus, determining the responsiveness of a cancer patient to a particular cancer treatment refers to identifying the cancer patient as having an increased or reduced likelihood of responding to the particular treatment. For example, an increased (or decreased) responsiveness to or an increased (or decreased) likelihood for responding to a cancer treatment provided to a cancer patient refers to an increase (or decrease) of, at least 5%, at least 10%, at least 15%, at least 20%, at least 50%, at least 100%, at least 200%, at least 300%, at least 400%, at least 500%, at least 1000% or more, in the effectiveness in reducing or decreasing the symptoms of the cancer when measured using any methods well-known in the art.

The term “predict”, “predictive” or “prediction” refers to the ability to assess the probable course and outcome of a therapeutic intervention, i.e. the likelihood of amelioration of or recovery from the disease, and includes determining or assessing the responsiveness or likelihood of responsiveness of the effectiveness of a cancer treatment in a patient. Thus, a predictive biomarker, i.e. its over- or underactivity, altered level, etc. before, during or after a treatment, provides information about the effect of a therapeutic intervention. Predicting the outcome of a treatment or the course of a treatment may be carried out (i) before the treatment has been initiated (or before the treatment period has progressed substantially) to assess the responsiveness of a patient, and/or (ii) during the course of the treatment to monitor responsiveness of a patient and adjust treatment schedules (administered dosages and frequency of administration) should the responsiveness change and/or (iii) after completion of a treatment to assess responsiveness of a patient for further treatments. The term “likelihood” when used in reference to the effectiveness of a patient tumor response generally refers to an increase in the probability that the effectiveness of the treatment is increasing and/or that the rate of tumor progress or tumor cell growth is decreasing, i.e. that the symptoms of the cancer being treated will be ameliorated or decreased.

The term “effective (tumor) response” used in reference to a patient or a subject refers to any increase in the therapeutic benefit to the patient. An “effective patient tumor response” can be, for example, about 5%, about 10%, about 25%, about 50%, or about 100% decrease in the rate of progress of the tumor and/or in the physical symptoms of a cancer. An “effective patient tumor response” can also be, for example, about 5%, about 10%, about 25%, about 50%, about 100%, about 200%, or more increase in the response of the patient, as measured by any suitable means, such as gene expression, cell counts, assay results, tumor size, etc. An improvement in the cancer or cancer-related disease can be characterized as a complete or partial response. The term “complete response” refers to an absence of clinically detectable disease with normalization of any previously abnormal radiographic studies, bone marrow, and cerebrospinal fluid (CSF) or abnormal monoclonal protein measurements. The term “partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The terms “determine”, “measuring”, “evaluate”, “assess” and “assay” as used herein with reference to the responsiveness to a specific cancer treatment with a GSPT1 negative modulator refers to analysing quantitatively and/or qualitatively if a marker is present or not in absolute or relative terms (i.e. relative to one or more previous analyses). Thus, assessing the presence of a marker can include determining the amount of the marker present, as well as determining whether it is present or absent. In any of the methods comprising the measurement of gene expression in a test tissue sample as disclosed herein, however, it should be understood that any step comprising the provision of a test tissue sample obtained from a subject is an optional step. It should also be understood that in certain embodiments, the “measuring” or “assessing” step to determine the expression level, phosphorylation level or gene amplification level includes a transformative method of assaying for gene expression, for example by performing a reverse transcriptase-polymerase chain reaction (RT-PCR) assay or an IHC assay. In some cases, the expression level, phosphorylation level or gene amplification level is assessed or determined by, for example, reviewing a report of test results from a laboratory or performed by a different individual or entity. In certain cases, the steps of the methods up to, and including, assessing gene expression provides an intermediate result that can be provided to a physician or other healthcare provider for use in selecting a suitable candidate for treatment with a GSPT1 negative modulator. In certain embodiments, the steps that provide the intermediate result is performed by a medical practitioner or someone acting under the direction of a medical practitioner. In other embodiments, these steps are performed by an independent laboratory or by an independent person such as a laboratory technician.

The terms “isolated” and “purified” refer to isolation of a molecule (e.g. a polynucleotide or polypeptide) such that the substance comprises a substantial portion of the sample in which it resides, i.e., greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. Techniques for purifying polynucleotides are well known in the art and include, for example, gel electrophoresis, ion-exchange chromatography, affinity chromatography, flow sorting, and sedimentation according to density.

The term “monitor” and “monitoring a treatment” (or therapy) for the purpose of the present disclosure refers to the overseeing, supervision, regulation, watching, tracking, surveilling or observing of the progression of the cancer in a subject who receives a treatment or therapy for the particular cancer, in particular a treatment with a GSPT1 negative modulator. Monitoring the effectiveness of the treatment allows to estimate at an early stage during the therapy whether the prescribed treatment is effective or not, and therefore to adjust the treatment regime accordingly (by halting the treatment or changing the treatment schedule, such as increasing or decreasing of dosage or frequency of administration, and the like). The monitoring can be performed, for example, by following the expression of mRNA or protein biomarkers.

The term “prevent”, “preventing”, “prevention”, or “prophylactic treatment” refers to a measure to which a subject is subjected to in order to reducing the probability of developing cancer in a subject, who does not have, but may or may not be at risk of or susceptible to developing cancer.

The term “treat,” “treating” or “treatment” refers to a measure to which a cancer patient is subjected to in order to reduce the severity of the cancer, or to slow down the progression of the cancer. An “effective patient response” refers to any increase in the therapeutic benefit to the patient such as (i) observable and/or measurable reduction in the number (or absence) of cancer cell and/or (ii) a reduction in the proliferation or survival of cancer cells; and/or (iii) cessation or reduction in the size of a tumor, and/or (iv) relief of one or more of the symptoms associated with the specific cancer and/or (v) reduced mortality, improved survival and/or progression-free survival and/or (vi) improved quality of life. Any of these parameters are readily measurable by routine procedures familiar to a skilled physician. In some embodiments, an “effective patient response” can be, for example, a 5%, 10%, 25%, 50%, or 100% change in the physical signs or symptoms of the cancer.

The term “sample” or “biological sample” or “test sample” as used herein refers to a cancer-affected or cancerous sample obtained from a subject or patient. This includes a sample of a tissue or of a fluid obtained from e.g. organs, tissues, fractions and cells isolated from a subject, in either healthy state, precancerous state or cancerous state. Exemplary biological samples include but are not limited to a cell lysate, cell culture, cell line, circulating cells, e.g. PBMCs (peripheral blood mononuclear cells), tissue, skin, oral tissue, gastrointestinal tissue, organ, organelle, biological fluid, blood sample, serum, plasma, buccal scrape, saliva, cerebrospinal fluid, urine, stool, or bone marrow, and the like. A biological sample can include a solid tissue sample (e.g., bone marrow) or a liquid sample (e.g., blood, whole blood, plasma, amniotic fluid, pleural fluid, peritoneal fluid, central spinal fluid, urine, saliva or other body fluid that contains cells). Samples of tissues, cells and the like may be obtained from any part of the body (externally or internally) by a biopsy. A biopsy may be performed either using open surgical techniques or minimally invasive/percutaneous techniques, such as e.g. fine needle aspiration (FNA), transbronchial needle aspiration (TBNA), or core biopsies. In some embodiments biological samples include but are not limited to whole blood, partially purified blood, urine, tissue biopsies, circulating cells, e.g. PBMCs, (including circulating cancer cells, such as circulating tumor cells, circulating stem cells and/or circulating epithelial-mesenchymal transition cells), and the like. The term “test cells” refer to cells obtained from a patient suffering from a cancer, e.g. a myc-driven cancer, which is being tested for its (positive or negative) responsiveness to a treatment with a GSPT1 negative modulator.

The term “control” or “reference” when used in combination with a sample refers to a non-cancerous sample which is suitable for comparison with a sample that is afflicted with a cancer, e.g. a myc-driven cancer, (also referred to as a test sample) as defined herein, or a sample that is afflicted with a non myc-driven cancer which is suitable for comparison with a sample that is afflicted with a myc-driven cancer. Such a non-cancerous sample may be obtained from a healthy subject not having cancer or from a non-cancerous tissue or fluid obtained from the cancer patient. The term “control” or “reference” when used in combination with a sample may also refer to a cancerous sample from a different cancer patient having been assessed and identified as a responder (i.e. showing good responsiveness) or non-responder to treatment with a GSPT1 negative modulator. In some embodiments the level of at least one of the identified biomarkers is determined in the test sample relative to the level of the at least one of the identified biomarkers determined the control sample. In some embodiments the level of at least one of the identified biomarkers is determined in the test sample relative to the level of a molecule other than the at least one of the identified biomarker molecule, determined in the control sample or the test sample itself. Such a molecule may be e.g. a housekeeping gene (or gene sets) or gene products therefrom, which refers to a gene (or gene sets) involved in basic functions needed for maintenance of the cell. Housekeeping genes are transcribed at a relatively constant level and are thus used to normalize gene levels that vary across different samples. Examples include e.g. GAPDH, β-glucuronidase (GUSB), actin, ubiquitin, tubulin, and the like.

Likewise, the term “control” or “reference” when used in combination with a level or a value refers to a separate baseline level or value measured in a non-cancerous sample, which is suitable for comparison with a test sample that is afflicted with a cancer, e.g. a myc-driven cancer, as defined herein. In some embodiments, a control or reference sample (or a control or reference level/value) may be obtained from the same subject afflicted with a cancer, e.g. a myc-driven cancer, as defined herein, of which the test sample is obtained from, but using a sample that is non-cancerous, such as a sample from a site remote of the cancer site. In some embodiments such a control sample is a tissue matched, non-cancerous sample. In some embodiments, a control or reference sample (or a control or reference level/value) may be obtained from the same subject afflicted with a cancer, e.g. a myc-driven cancer, as defined herein, of which the test sample is obtained from, using a sample that is afflicted with the cancer but the sample is taken at an earlier point in time. This applies for example when the course of a treatment with a GSPT1 negative modulator is monitored and a first sample taken before or at a first time point during treatment is used as a control or reference sample for a test sample taken at a later, second time point during treatment. In some embodiments, a control or reference sample (or a control or reference level/value) may be obtained from a single or a plurality of subjects who are not afflicted with the cancer, i.e. a healthy subject. In some embodiments, a control or reference sample (or a control or reference level/value) may further be obtained from a single or a plurality of subjects who are afflicted with the same cancer, e.g. a myc-driven cancer, as defined herein, and for which prediction of responsiveness was already established, for example from patients with a good responsiveness, i.e. from responders. A control or reference level/value can be an absolute or a relative level/value; a level/value that has an upper and/or lower limit; a range of levels/values; an average or a median or a mean level/value, or a level/value as compared to a different control or baseline level/value. A control or reference level/value can be based on a single sample or a large number of samples.

In one aspect the disclosure provides an in vitro method to determine or assess the responsiveness of a cancer patient to a treatment with at least one GSPT1 negative modulator, comprising the steps of
(i) obtaining a cancerous sample from the patient,
(ii) determining the level of one or more biomarkers selected from a myc transcription factor markers or surrogate marker thereof, such as a translation addicted marker as defined herein and combinations thereof, such as one or more of EIF4EBP1, EIF4EBP2, L-Myc, N-Myc and c-Myc, or combinations thereof in the cancerous sample,
(iii) comparing the level of the one or more biomarkers determined in step (ii) with the level of one or more reference markers determined in a control sample, and
(iv) identifying the patient as having an increased or decreased responsiveness to the treatment if the level of the one or more biomarkers in the cancerous sample is altered as compared to the level of the one or more reference markers in the control sample.

In some embodiments, the cancer is a myc-driven cancer.

In some embodiments, the cancer is a blood-borne tumor cancer, such as a hematological cancer, such as a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphoma.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and the control sample.

Expression levels are generally normalized using one or more housekeeping genes (or gene sets) or gene products therefrom, which refers to a gene (or gene sets) involved in basic functions needed for maintenance of the cell, and which are transcribed at a relatively constant level and are thus used to normalize gene levels that vary across different samples, e.g. GAPDH, β-glucuronidase (GUSB), actin, ubiquitin, tubulin, and the like.

In some embodiments, the control sample is obtained from a healthy subject. In some embodiments, the control sample is a non-cancerous biological sample obtained from the cancer patient, i.e., from a tissue or body part not affected by the cancer, such as from a tissue matched healthy sample. In some embodiments, the control sample is obtained from the cancer patient during a treatment with a GSPT1 negative modulator, and is a cancerous biological sample taken prior to treatment or at an earlier time point during the treatment. In some embodiments, the control sample is obtained from a different cancer patient, i.e. a cancer patient other than the cancer patient of which the cancerous sample has been obtained, which has previously been determined to be a responder or a non-responder.

In some embodiments, the method to determine or assess the responsiveness of a cancer patient to a treatment with a GSPT1 negative modulator is carried out before the cancer patient is subjected to the treatment with a GSPT1 negative modulator. In some embodiments, the method is carried out during the cancer patient is subjected to the treatment with a GSPT1 negative modulator. In some embodiments, the method is carried out after the cancer patient has been subjected to the treatment with a GSPT1 negative modulator.

In some embodiments, the biomarker used in the methods of the disclosure is N-Myc. In some embodiments, the biomarker used in the methods of the disclosure is N-Myc and the myc-driven cancer to be treated is lymphoma, AML or MM.

Thus, in some embodiments the disclosure provides an in vitro method to determine or assess the responsiveness of a cancer patient, such as a patent suffering from AML or MM, to a treatment with at least one GSPT1 negative modulator as defined herein, comprising the steps of
(i) obtaining a cancerous sample from the patient,
(ii) determining the level of N-Myc in the cancerous sample,
(iii) comparing the level of N-Myc determined in step (ii) with the level of one or more reference markers determined in a control sample, and
(iv) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

In some embodiments, the biomarker used in the methods of the disclosure is L-Myc. In some embodiments, the biomarker used in the methods of the disclosure is L-Myc and the myc-driven cancer to be treated is lymphoma, AML or MM.

In some embodiments, the biomarker used in the methods of the disclosure is c-Myc. In some embodiments, the biomarker used in the methods of the disclosure is c-Myc and the myc-driven cancer to be treated is lymphoma, AML or MM.

Thus, in some embodiments the disclosure provides an in vitro method to determine or assess the responsiveness of a cancer patient, such as a patient suffering from AML or MM, to a treatment with at least one GSPT1 negative modulator as defined herein, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a control sample, and
(iv) identifying the patient as having an increased responsiveness to the treatment if the level of L-Myc in the cancerous sample is equal to or lower as compared to the level of the one or more reference markers in the control sample or identifying the patient as having a reduced responsiveness to the treatment if the level of L-Myc in the cancerous sample is elevated as compared to the level of the one or more reference markers in the control sample.

In some embodiments, the biomarker used in the methods of the disclosure is N-Myc and L-Myc. In some embodiments, the biomarker used in the methods of the disclosure is N-Myc and L-Myc and the myc-driven cancer to be treated is AML or MM.

Thus, in some embodiments the disclosure provides an in vitro method to determine or assess the responsiveness of a cancer patient, such as a patent suffering from AMI or MM, to a treatment with at least one GSPT1 negative modulator as defined herein, comprising the steps of
(i) obtaining a cancerous sample from the patient,
(ii) determining the level of N-Myc and L-Myc in the cancerous sample,
(iii) comparing the level of N-Myc and L-Myc determined in step (ii) with the level of one or more reference markers determined in a control sample, and
(iv) identifying the patient as having an increased responsiveness to the treatment if the level of N-Myc in the cancerous sample is elevated and the level of L-Myc in the cancerous sample is equal to or lower as compared to the level of the one or more reference markers in the control sample or identifying the patient as having a reduced responsiveness to the treatment if the level of N-Myc in the cancerous sample is equal to or lower and the level of L-Myc in the cancerous sample is elevated as compared to the level of the one or more reference markers in the control sample.

In one aspect, the disclosure also provides a method of treating a cancer patient with a therapeutically effective amount of at least one GSPT1 negative modulator comprising:
(i) obtaining a cancerous sample from the patient,
(ii) determining the level of one or more biomarkers selected from a myc transcription factor marker or surrogate marker thereof, such as a translation addicted marker as defined herein, e.g. one or more of EIF4EBP1, EIF4EBP2, L-Myc, N-Myc and c-Myc, or combinations thereof, in the cancerous sample,
(iii) comparing the level of the one or more biomarkers determined in step (ii) with the level of one or more reference markers determined in a control sample,
(iv) identifying the patient as having an increased or decreased responsiveness to a treatment with a GSPT1 negative modulator if the level of the one or more biomarkers in the cancerous sample is altered in comparison to the level of the one or more reference markers in the control sample, and
(v) administering to the patient having an increased responsiveness to the treatment with a GSPT1 negative modulator the therapeutically effective amount of a GSPT1 negative modulator.

In some embodiments, the cancer is a myc-driven cancer.

In some embodiments, the cancer is a blood-borne tumor cancer, such as a hematological cancer, such as a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphoma.

In one aspect, the disclosure also provides a method of treating hematological cancer with an elevated level in EIF4EBP1, EIF4EBP2, L-Myc, N-Myc and/or c-Myc with a therapeutically effective amount of at least one GSPT1 negative modulator.

In some embodiments, the method is treating AML with an elevated level in N-Myc with a therapeutically effective amount of at least one GSPT1 negative modulator. In some embodiments, the method is treating AML with an elevated level in N-Myc and decreased level in L-Myc with a therapeutically effective amount of at least one GSPT1 negative modulator.

In some embodiments, the method is treating MM with an elevated level in N-Myc with a therapeutically effective amount of at least one GSPT1 negative modulator. In some embodiments, the method is treating MM with an elevated level in N-Myc and decreased level in L-Myc with a therapeutically effective amount of at least one GSPT1 negative modulator.

In some embodiments, the method is treating ALL with an elevated level in N-Myc with a therapeutically effective amount of at least one GSPT1 negative modulator. In some embodiments, the method is treating ALL with an elevated level in N-Myc and decreased level in L-Myc with a therapeutically effective amount of at least one GSPT1 negative modulator.

In some embodiments, the method is treating CLL with an elevated level in N-Myc with a therapeutically effective amount of at least one GSPT1 negative modulator. In some embodiments, the method is treating CLL with an elevated level in N-Myc and decreased level in L-Myc with a therapeutically effective amount of at least one GSPT1 negative modulator.

In some embodiments, the method is treating CML with an elevated level in N-Myc with a therapeutically effective amount of at least one GSPT1 negative modulator. In some embodiments, the method is treating CML with an elevated level in N-Myc and decreased level in L-Myc with a therapeutically effective amount of at least one GSPT1 negative modulator.

In some embodiments, the method is treating Hodgkin's lymphoma with an elevated level in N-Myc with a therapeutically effective amount of at least one GSPT1 negative modulator.

In some embodiments, the method is treating Hodgkin's lymphoma with an elevated level in N-Myc and decreased level in L-Myc with a therapeutically effective amount of at least one GSPT1 negative modulator.

In some embodiments, the method is treating non-Hodgkin's lymphoma with an elevated level in N-Myc with a therapeutically effective amount of at least one GSPT1 negative modulator. In some embodiments, the method is treating non-Hodgkin's lymphoma with an elevated level in N-Myc and decreased level in L-Myc with a therapeutically effective amount of at least one GSPT1 negative modulator.

In some embodiments, the cancer is a solid tumor cancer, such as breast cancer, colorectal cancer, lung cancer, e.g. SCLC, NSCLC, liver cancer, stomach cancer, pancreatic cancer, neuroendocrine cancer, e.g., neuroendocrine prostate cancer (for example, NEPC (castration-resistant neuroendocrine prostate cancer)) and lung neuroendocrine tumors (Lu-NETs), gastric cancer, esophageal cancer, bladder cancer, skin cancer, and head and neck cancer.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and the control sample.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and a molecule other than the marker molecule, which can be used for determining a reference or control value is determined in the control sample. Molecules, whose level can be used as reference or control values include any housekeeping gene (or gene sets) or gene products therefrom, which refers to a gene (or gene sets) involved in basic functions needed for maintenance of the cell, and which are transcribed at a relatively constant level and are thus used to normalize gene levels that vary across different samples, e.g. GAPDH, β-glucuronidase (GUSB), actin, ubiquitin, tubulin, and the like.

In some embodiments, the control sample is obtained from a healthy subject. In some embodiments, the control sample is a non-cancerous biological sample obtained from the cancer patient, i.e. from a tissue or body part not affected by the cancer, such as from a tissue matched healthy sample. In some embodiments, the control sample is obtained from the cancer patient during a treatment with a GSPT1 negative modulator, and is a cancerous biological sample taken prior to treatment or at an earlier time point during the treatment. In some embodiments, the control sample is obtained from a different cancer patient, i.e. a cancer patient other than the cancer patient of which the cancerous sample has been obtained, which has previously been determined to be a responder or a non-responder.

In some embodiments, the method to determine or assess the responsiveness of a cancer patient to a treatment with a GSPT1 negative modulator is carried out before the cancer patient is subjected to the treatment with a GSPT1 negative modulator. In some embodiments, the method is carried out during the cancer patient is subjected to the treatment with a GSPT1 negative modulator. In some embodiments, the method is carried out after the cancer patient has been subjected to the treatment with a GSPT1 negative modulator.

In some embodiments, the biomarker used in the methods of the disclosure is EIF4EBP1. In some embodiments, the biomarker used in the methods of the disclosure is EIF4EBP1 and the cancer, e.g., myc-driven cancer, to be treated is breast cancer or SCLC. In some embodiments, the biomarker used in the methods of the disclosure is EIF4EBP1 and the cancer, e.g., myc-driven cancer, to be treated is breast cancer.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of EIF4EBP1 expression or phosphorylation in the cancerous sample,
(iii) comparing the level of EIF4EBP1 expression or phosphorylation determined in step (ii) with the level of one or more reference markers determined in a control sample, and
(iv) identifying the patient as having an increased responsiveness to the treatment if the level of EIF4EBP1 expression or phosphorylation in the cancerous sample is elevated as compared to the level of the one or more reference markers in the control sample or identifying the patient as having a reduced responsiveness to the treatment if the level of EIF4EBP1 expression or phosphorylation in the cancerous sample is equal to or lower as compared to the level of the one or more reference markers in the control sample.

In some embodiments, the biomarker used in the methods of the disclosure is EIF4EBP2. In some embodiments, the biomarker used in the methods of the disclosure is EIF4EBP2 and the cancer, e.g., myc-driven cancer, to be treated is breast cancer or SCLC. In some embodiments, the biomarker used in the methods of the disclosure is EIF4EBP2 and the cancer, e.g., myc-driven cancer, to be treated is breast cancer.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of EIF4EBP2 expression or phosphorylation in the cancerous sample,
(iii) comparing the level of EIF4EBP2 expression or phosphorylation determined in step (ii) with the level of one or more reference markers determined in a control sample, and
(iv) identifying the patient as having an increased responsiveness to the treatment if the level of EIF4EBP2 expression or phosphorylation in the cancerous sample is elevated as compared to the level of the one or more reference markers in the control sample or identifying the patient as having a reduced responsiveness to the treatment if the level of EIF4EBP2 expression or phosphorylation in the cancerous sample is equal to or lower as compared to the level of the one or more reference markers in the control sample.

In some embodiments, the biomarker used in the methods of the disclosure is L-Myc. In some embodiments, the biomarker used in the methods of the disclosure is L-Myc and the cancer, e.g., myc-driven cancer, to be treated is breast cancer or SCLC. In some embodiments, the biomarker used in the methods of the disclosure is L-Myc and the cancer, e.g., myc-driven cancer, to be treated is SCLC.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a control sample, and
(iv) identifying the patient as having an increased responsiveness to the treatment if the level of L-Myc in the cancerous sample is elevated as compared to the level of the one or more reference markers in the control sample or identifying the patient as having a reduced responsiveness to the treatment if the level of L-Myc in the cancerous sample is equal to or lower as compared to the level of the one or more reference markers in the control sample.

In some embodiments, a combination of biomarkers is used in the methods of the disclosure, which include EIF4EBP1 and L-Myc. In some embodiments, a combination of biomarkers is used in the methods of the disclosure, which include EIF4EBP1 and L-Myc and the myc-driven cancer to be treated is breast cancer or SCLC.

In some embodiments, a combination of biomarkers is used in the methods of the disclosure, which include EIF4EBP2 and L-Myc. In some embodiments, a combination of biomarkers is used in the methods of the disclosure, which include EIF4EBP2 and L-Myc and the myc-driven cancer to be treated is breast cancer or SCLC.

In some embodiments, a combination of biomarkers is used in the methods of the disclosure, which include EIF4EBP1 and EIF4EBP2. In some embodiments, a combination of biomarkers is used in the methods of the disclosure, which include EIF4EBP1 and EIF4EBP2 and the myc-driven cancer to be treated is breast cancer or SCLC.

In some embodiments, a combination of biomarkers is used in the methods of the disclosure, which include EIF4EBP1, EIF4EBP2 and L-Myc. In some embodiments, a combination of biomarkers is used in the methods of the disclosure, which include EIF4EBP1, EIF4EBP2 and L-Myc and the myc-driven cancer to be treated is breast cancer or SCLC.

Thus, in some embodiments the disclosure provides an in vitro method to determine or assess the responsiveness of a cancer patient to a treatment with at least one GSPT1 negative modulator as defined herein, comprising the steps of
(i) obtaining a cancerous sample from the patient,
(ii) determining the level of EIF4EBP1 expression or phosphorylation and/or EIF4EBP2 and/or L-Myc in the cancerous sample,
(iii) comparing the level of the EIF4EBP1 and/or EIF4EBP2 and/or L-Myc determined in step (ii) with the level of one or more reference markers determined in a control sample, and
(iv) identifying the patient as having an increased responsiveness to the treatment if the level of EIF4EBP1 expression or phosphorylation and/or EIF4EBP2 and/or L-Myc in the cancerous sample is elevated as compared to the level of the one or more reference markers in the control sample or identifying the patient as having a reduced responsiveness to the treatment if the level of EIF4EBP1 expression or phosphorylation and/or EIF4EBP2 and/or L-Myc in the cancerous sample is equal to or lower as compared to the level of the one or more reference markers in the control sample.

In some embodiments, the biomarker used in the methods of the disclosure is N-Myc. In some embodiments, the biomarker used in the methods of the disclosure is N-Myc and the cancer, myc-driven cancer, to be treated is NSCLC.

In some embodiments the disclosure provides an in vitro method to determine or assess the responsiveness of a cancer patient, such as a patient suffering from NSCLC, to a treatment with at least one GSPT1 negative modulator as defined herein, comprising the steps of
(i) obtaining a cancerous sample from the patient,
(ii) determining the level of N-Myc thereof in the cancerous sample,
(iii) comparing the level of N-Myc determined in step (ii) with the level of one or more reference markers determined in a control sample, and
(iv) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

In some embodiments, the biomarker used in the methods of the disclosure is N-Myc. In some embodiments, the biomarker used in the methods of the disclosure is N-Myc and the myc-driven cancer to be treated is a neuroendocrine cancer, for example, neuroendocrine prostate cancer (for example, NEPC (castration-resistant neuroendocrine prostate cancer)) and lung neuroendocrine tumors (Lu-NETs).

Thus, in some embodiments the disclosure provides an in vitro method to determine or assess the responsiveness of a cancer patient, such as a patent suffering from a neuroendocrine cancer, for example, neuroendocrine prostate cancer (for example, NEPC (castration-resistant neuroendocrine prostate cancer)) and lung neuroendocrine tumors (Lu-NETs), to a treatment with at least one GSPT1 negative modulator as defined herein, comprising the steps of
(v) obtaining a cancerous sample from the patient,
(vi) determining the level of N-Myc in the cancerous sample,
(vii) comparing the level of N-Myc determined in step (ii) with the level of one or more reference markers determined in a control sample, and
(viii) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

In one aspect, the disclosure also provides a method of treating a cancer patient with a therapeutically effective amount of at least one GSPT1 negative modulator comprising:
(vi) obtaining a cancerous sample from the patient,
(vii) determining the level of one or more biomarkers selected from a myc transcription factor marker or surrogate marker thereof, such as a translation addicted marker as defined herein, e.g. one or more of EIF4EBP1, EIF4EBP2, L-Myc, N-Myc and c-Myc, or combinations thereof, in the cancerous sample,
(viii) comparing the level of the one or more biomarkers determined in step (ii) with the level of one or more reference markers determined in a control sample,
(ix) identifying the patient as having an increased or decreased responsiveness to a treatment with a GSPT1 negative modulator if the level of the one or more biomarkers in the cancerous sample is altered in comparison to the level of the one or more reference markers in the control sample, and
(x) administering to the patient having an increased responsiveness to the treatment with a GSPT1 negative modulator the therapeutically effective amount of a GSPT1 negative modulator.

In some embodiments, the cancer is a myc-driven cancer.

In some embodiments, the cancer is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, and head and neck cancer.

In one aspect, the disclosure also provides a method of treating a cancer patient with an elevated level in EIF4EBP1, EIF4EBP2, L-Myc, N-Myc and/or c-Myc with a therapeutically effective amount of at least one GSPT1 negative modulator. In some embodiments, the method is treating breast cancer with an elevated level in EIF4EBP1 with a therapeutically effective amount of at least one GSPT1 negative modulator. In some embodiments, the method is treating SCLC with an elevated level in L-Myc with a therapeutically effective amount of at least one GSPT1 negative modulator. In some embodiments, the method is treating NSCLC with an elevated level in N-Myc with a therapeutically effective amount of at least one GSPT1 negative modulator. In some embodiments, the method is treating gastric cancer with an elevated level in N-Myc with a therapeutically effective amount of at least one GSPT1 negative modulator. In some embodiments, the method is treating liver cancer with an elevated level in N-Myc with a therapeutically effective amount of at least one GSPT1 negative modulator. In some embodiments, the method is treating neuroendocrine tumors with an elevated level in N-Myc with a therapeutically effective amount of at least one GSPT1 negative modulator. In some embodiments, the method is treating NEPC with an elevated level in N-Myc with a therapeutically effective amount of at least one GSPT1 negative modulator. In some embodiments, the method is treating lung neuroendocrine tumors (Lu-NETs) with an elevated level in N-Myc with a therapeutically effective amount of at least one GSPT1 negative modulator.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and the control sample.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and a molecule other than the marker molecule, which can be used for determining a reference or control value is determined in the control sample. Molecules, whose level can be used as reference or control values include any housekeeping gene (or gene sets) or gene products therefrom, which refers to a gene (or gene sets) involved in basic functions needed for maintenance of the cell, and which are transcribed at a relatively constant level and are thus used to normalize gene levels that vary across different samples, e.g. GAPDH, β-glucuronidase (GUSB), actin, ubiquitin, tubulin, and the like.

In some embodiments, the control sample is obtained from a healthy subject. In some embodiments, the control sample is a non-cancerous biological sample obtained from the cancer patient, i.e. from a tissue or body part not affected by the cancer, such as from a tissue matched healthy sample. In some embodiments, the control sample is obtained from the cancer patient during a treatment with a GSPT1 negative modulator, and is a cancerous biological sample taken prior to treatment or at an earlier time point during the treatment. In some embodiments, the control sample is obtained from a different cancer patient, i.e. a cancer patient other than the cancer patient of which the cancerous sample has been obtained, which has previously been determined to be a responder or a non-responder.

In some embodiments, the method to determine or assess the responsiveness of a cancer patient to a treatment with a GSPT1 negative modulator is carried out before the cancer patient is subjected to the treatment with a GSPT1 negative modulator. In some embodiments, the method is carried out during the cancer patient is subjected to the treatment with a GSPT1 negative modulator. In some embodiments, the method is carried out after the cancer patient has been subjected to the treatment with a GSPT1 negative modulator.

In some embodiments, the biomarker used in the methods of the disclosure is N-Myc. In some embodiments, the biomarker used in the methods of the disclosure is N-Myc and the myc-driven cancer to be treated is AML or MM.

Thus, in some embodiments the disclosure provides a method of treating a cancer patient, such as a patent suffering from AML or MM, with a therapeutically effective amount of at least one GSPT1 negative modulator as defined herein, comprising the steps of
(i) obtaining a cancerous sample from the patient,
(ii) determining the level of N-Myc in the cancerous sample,
(iii) comparing the level of N-Myc determined in step (ii) with the level of one or more reference markers determined in a control sample, and
(iv) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is L-Myc. In some embodiments, the biomarker used in the methods of the disclosure is L-Myc and the myc-driven cancer to be treated is AML or MM.

Thus, in some embodiments the disclosure provides a method of treating a cancer patient, such as a patent suffering from AML or MM, with a therapeutically effective amount of at least one GSPT1 negative modulator as defined herein, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a control sample, and
(iv) identifying the patient as having an increased responsiveness to the treatment if the level of L-Myc in the cancerous sample is equal to or lower as compared to the level of the one or more reference markers in the control sample or identifying the patient as having a reduced responsiveness to the treatment if the level of L-Myc in the cancerous sample is elevated as compared to the level of the one or more reference markers in the control sample and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is N-Myc and L-Myc. In some embodiments, the biomarker used in the methods of the disclosure is N-Myc and L-Myc and the myc-driven cancer to be treated is AML or MM.

Thus, in some embodiments the disclosure provides a method of treating a cancer patient, such as a patent suffering from AML or MM, with a therapeutically effective amount of at least one GSPT1 negative modulator as defined herein, comprising the steps of
(i) obtaining a cancerous sample from the patient,
(ii) determining the level of N-Myc and L-Myc in the cancerous sample,
(iii) comparing the level of N-Myc and L-Myc determined in step (ii) with the level of one or more reference markers determined in a control sample, and
(iv) identifying the patient as having an increased responsiveness to the treatment if the level of N-Myc in the cancerous sample is elevated and the level of L-Myc in the cancerous sample is equal to or lower as compared to the level of the one or more reference markers in the control sample or identifying the patient as having a reduced responsiveness to the treatment if the level of N-Myc in the cancerous sample is equal to or lower and the level of L-Myc in the cancerous sample is elevated as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In one aspect, the disclosure also provides a use of one or more biomarkers selected from a myc transcription factor marker or surrogate marker thereof, such as a translation addicted marker as defined herein, e.g. one or more of EIF4EBP1, EIF4EBP2, L-Myc, N-Myc and c-Myc, or combinations thereof, to evaluate the responsiveness of a cancer patient to a treatment with a GSPT1 negative modulator, wherein an altered level of the one or more biomarkers in a cancerous sample obtained from said patient compared to a control sample is indicative that said patient has an increased or decreased likelihood of responsiveness to the treatment with a GSPT1 negative modulator.

In some embodiments, the cancer is a myc-driven cancer.

In some embodiments, the cancer is a blood-borne tumor cancer, such as a hematological cancer, such as a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphoma.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and the control sample.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and a molecule other than the marker molecule, which can be used for determining a reference or control value is determined in the control sample. Molecules, whose level can be used as reference or control values include any housekeeping gene (or gene sets) or gene products therefrom, which refers to a gene (or gene sets) involved in basic functions needed for maintenance of the cell, and which are transcribed at a relatively constant level and are thus used to normalize gene levels that vary across different samples, e.g. GAPDH, β-glucuronidase (GUSB), actin, ubiquitin, tubulin, and the like.

In some embodiments, the control sample is obtained from a healthy subject. In some embodiments, the control sample is a non-cancerous biological sample obtained from the cancer patient, i.e. from a tissue or body part not affected by the cancer, such as from a tissue matched healthy sample. In some embodiments, the control sample is obtained from the cancer patient during a treatment with a GSPT1 negative modulator, and is a cancerous biological sample taken prior to treatment or at an earlier time point during the treatment. In some embodiments, the control sample is obtained from a different cancer patient, i.e. a cancer patient other than the cancer patient of which the cancerous sample has been obtained, which has previously been determined to be a responder or a non-responder.

In some embodiments, the method to determine or assess the responsiveness of a cancer patient to a treatment with a GSPT1 negative modulator is carried out before the cancer patient is subjected to the treatment with a GSPT1 negative modulator. In some embodiments, the method is carried out during the cancer patient is subjected to the treatment with a GSPT1 negative modulator. In some embodiments, the method is carried out after the cancer patient has been subjected to the treatment with a GSPT1 negative modulator.

In some embodiments, the biomarker according to the disclosure is N-Myc. In some embodiments, the biomarker used in the methods of the disclosure is N-Myc and the myc-driven cancer to be treated is AML or MM.

Thus, in some embodiments the disclosure provides a use of N-Myc to evaluate the responsiveness of a cancer patient, such as a patient suffering from AML or MM, to a treatment with a GSPT1 negative modulator, comprising the steps of
(i) obtaining a cancerous sample from the patient,
(ii) determining the level of N-Myc in the cancerous sample,
(iii) comparing the level of N-Myc determined in step (ii) with the level of one or more reference markers determined in a control sample, and
(iv) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

In some embodiments, the biomarker according to the disclosure is L-Myc. In some embodiments, the biomarker used in the methods of the disclosure is L-Myc and the myc-driven cancer to be treated is AML or MM.

Thus, in some embodiments the disclosure provides a use of L-Myc to evaluate the responsiveness of a cancer patient, such as a patient suffering from AML or MM, to a treatment with a GSPT1 negative modulator, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a control sample, and
(iv) identifying the patient as having an increased responsiveness to the treatment if the level of L-Myc in the cancerous sample is equal to or lower as compared to the level of the one or more reference markers in the control sample or identifying the patient as having a reduced responsiveness to the treatment if the level of L-Myc in the cancerous sample is elevated as compared to the level of the one or more reference markers in the control sample.

In some embodiments, the biomarker used according to the disclosure is N-Myc and L-Myc.

In some embodiments, the biomarker used in the methods of the disclosure is N-Myc and L-Myc and the myc-driven cancer to be treated is AML or MM.

Thus, in some embodiments the disclosure provides a use of N-Myc and L-Myc to evaluate the responsiveness of a cancer patient, such as a patient suffering from AML or MM, to a treatment with a GSPT1 negative modulator, comprising the steps of
(i) obtaining a cancerous sample from the patient,
(ii) determining the level of N-Myc and L-Myc in the cancerous sample,
(iii) comparing the level of N-Myc and L-Myc determined in step (ii) with the level of one or more reference markers determined in a control sample, and
(iv) identifying the patient as having an increased responsiveness to the treatment if the level of N-Myc in the cancerous sample is elevated and the level of L-Myc in the cancerous sample is equal to or lower as compared to the level of the one or more reference markers in the control sample or identifying the patient as having a reduced responsiveness to the treatment if the level of N-Myc in the cancerous sample is equal to or lower and the level of L-Myc in the cancerous sample is elevated as compared to the level of the one or more reference markers in the control sample.

In some embodiments, the biomarker used in the methods of the disclosure is EIF4EBP1. In some embodiments, the biomarker used in the methods of the disclosure is EIF4EBP1 and the cancer, e.g., myc-driven cancer, to be treated is breast cancer or SCLC. In some embodiments, the biomarker used in the methods of the disclosure is EIF4EBP1 and the cancer, e.g., myc-driven cancer, to be treated is breast cancer.

Thus, in some embodiments the disclosure provides a method of treating a cancer patient, such as a patient suffering from breast cancer or SCLC, with a therapeutically effective amount of at least one GSPT1 negative modulator comprising:
(i) obtaining a cancerous sample from the patient,
(ii) determining the level of EIF4EBP1 expression or phosphorylation in the cancerous sample,
(iii) comparing the level of EIF4EBP1 expression or phosphorylation determined in step (ii) with the level of one or more reference markers determined in a control sample,
(iv) identifying the patient as having an increased responsiveness to a treatment with a GSPT1 negative modulator if the level of EIF4EBP1 expression or phosphorylation in the cancerous sample is elevated in comparison to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is EIF4EBP2. In some embodiments, the biomarker used in the methods of the disclosure is EIF4EBP2 and the cancer, e.g., myc-driven cancer to be treated is breast cancer or SCLC. In some embodiments, the biomarker used in the methods of the disclosure is EIF4EBP2 and the cancer, e.g., myc-driven cancer, to be treated is breast cancer.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of EIF4EBP2 expression or phosphorylation in the cancerous sample,
(iii) comparing the level of EIF4EBP2 expression or phosphorylation determined in step (ii) with the level of one or more reference markers determined in a control sample, and
(iv) identifying the patient as having an increased responsiveness to the treatment if the level of EIF4EBP2 expression or phosphorylation in the cancerous sample is elevated as compared to the level of the one or more reference markers in the control sample or identifying the patient as having a reduced responsiveness to the treatment if the level of EIF4EBP2 expression or phosphorylation in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is L-Myc. In some embodiments, the biomarker used in the methods of the disclosure is L-Myc and the cancer, e.g., myc-driven cancer to be treated is breast cancer or SCLC. In some embodiments, the biomarker used in the methods of the disclosure is L-Myc and the cancer, e.g., myc-driven cancer, to be treated is SCLC.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a sample,
(iv) identifying the patient as having an increased responsiveness to a treatment with a GSPT1 negative modulator if the level of L-Myc in the cancerous sample is elevated in comparison to the level of the one or more reference markers in the control sample or identifying the patient as having a reduced responsiveness to the treatment if the level of L-Myc in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, a combination of biomarkers is used in the methods of the disclosure, which include EIF4EBP1 and L-Myc. In some embodiments, a combination of biomarkers is used in the methods of the disclosure, which include EIF4EBP1 and L-Myc and the cancer, e.g., myc-driven cancer, to be treated is breast cancer or SCLC.

In some embodiments, a combination of biomarkers is used in the methods of the disclosure, which include EIF4EBP2 and L-Myc. In some embodiments, a combination of biomarkers is used in the methods of the disclosure, which include EIF4EBP2 and L-Myc and the cancer, e.g., myc-driven cancer, to be treated is breast cancer or SCLC.

In some embodiments, a combination of biomarkers is used in the methods of the disclosure, which include EIF4EBP1 and EIF4EBP2. In some embodiments, a combination of biomarkers is used in the methods of the disclosure, which include EIF4EBP1 and EIF4EBP2 and the cancer, e.g., myc-driven cancer, to be treated is breast cancer or SCLC.

In some embodiments, a combination of biomarkers is used in the methods of the disclosure, which include EIF4EBP1, EIF4EBP2 and L-Myc. In some embodiments, a combination of biomarkers is used in the methods of the disclosure, which include EIF4EBP1, EIF4EBP2 and L-Myc and the cancer, e.g., myc-driven cancer, to be treated is breast cancer or SCLC.

Thus, in some embodiments the disclosure provides an in vitro method to determine or assess the responsiveness of a cancer patient, such as a patient suffering from breast cancer or SCLC, to a treatment with at least one GSPT1 negative modulator as defined herein, comprising the steps of
(i) obtaining a cancerous sample from the patient,
(ii) determining the level of EIF4EBP1 expression or phosphorylation and/or EIF4EBP2 and/or L-Myc in the cancerous sample,
(iii) comparing the level of EIF4EBP1 expression or phosphorylation and/or EIF4EBP2 and/or L-Myc determined in step (ii) with the level of one or more reference markers determined in a control sample,
(iv) identifying the patient as having an increased responsiveness to a treatment with a GSPT1 negative modulator if the level of EIF4EBP1 expression or phosphorylation and/or EIF4EBP2 and/or L-Myc in the cancerous sample is elevated in comparison to the level of the one or more reference markers in the control sample or identifying the patient as having a reduced responsiveness to the treatment if the level of EIF4EBP1 expression or phosphorylation and/or EIF4EBP2 and/or L-Myc in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is N-Myc. In some embodiments, the biomarker used in the methods of the disclosure is N-Myc and the cancer, e.g., myc-driven cancer, to be treated is NSCLC.

Thus, in some embodiments the disclosure provides a method of treating a cancer patient, such as a patient suffering from NSCLC, with a therapeutically effective amount of at least one GSPT1 negative modulator comprising:
(i) obtaining a cancerous sample from the patient,
(ii) determining the level of N-Myc in the cancerous sample,
(iii) comparing the level of N-Myc determined in step (ii) with the level of one or more reference markers determined in a control sample,
(iv) identifying the patient as having an increased responsiveness to a treatment with a GSPT1 negative modulator if the level of N-Myc in the cancerous sample is elevated in comparison to the level of the one or more reference markers in the control sample or identifying the patient as having a reduced responsiveness to the treatment if the level of N-Myc in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In some embodiments, the biomarker used in the methods of the disclosure is N-Myc. In some embodiments, the biomarker used in the methods of the disclosure is N-Myc and the cancer, e.g., myc-driven cancer to be treated is a neuroendocrine cancer, for example, neuroendocrine prostate cancer (for example, NEPC (castration-resistant neuroendocrine prostate cancer)) and lung neuroendocrine tumors (Lu-NETs).

Thus, in some embodiments the disclosure provides a method of treating a cancer patient, such as a patient suffering from a neuroendocrine cancer, for example, neuroendocrine prostate cancer (for example, NEPC (castration-resistant neuroendocrine prostate cancer)) and lung neuroendocrine tumors (Lu-NETs), with a therapeutically effective amount of at least one GSPT1 negative modulator comprising:
(i) obtaining a cancerous sample from the patient,
(ii) determining the level of N-Myc in the cancerous sample,
(iii) comparing the level of N-Myc determined in step (ii) with the level of one or more reference markers determined in a control sample,
(iv) identifying the patient as having an increased responsiveness to a treatment with a GSPT1 negative modulator if the level of N-Myc in the cancerous sample is elevated in comparison to the level of the one or more reference markers in the control sample or identifying the patient as having a reduced responsiveness to the treatment if the level of N-Myc in the cancerous sample is equal to or lower as compared to the level of the one or more reference markers in the control sample, and
(v) administering to the patient identified in step (v) as having an increased responsiveness to the treatment with a GSPT1 negative modulator the therapeutically effective amount of a GSPT1 negative modulator.

In one aspect, the disclosure also provides a use of one or more biomarkers selected from a myc transcription factor marker or surrogate marker thereof, such as a translation addicted marker as defined herein, e.g. one or more of EIF4EBP1, EIF4EBP2, L-Myc, N-Myc and c-Myc, or combinations thereof, to evaluate the responsiveness of a cancer patient to a treatment with a GSPT1 negative modulator, wherein an altered level of the one or more biomarkers in a cancerous sample obtained from said patient compared to a control sample is indicative that said patient has an increased or decreased likelihood of responsiveness to the treatment with a GSPT1 negative modulator.

In some embodiments, the cancer is a myc-driven cancer.

In some embodiments, the cancer is a solid tumor cancer, such as breast cancer, colorectal cancer, lung cancer, e.g. SCLC, NSCLC, liver cancer, neuroendocrine cancer, e.g., neuroendocrine prostate cancer (for example, NEPC (castration-resistant neuroendocrine prostate cancer)) and lung neuroendocrine tumors (Lu-NETs), stomach cancer, pancreatic cancer, gastric cancer, esophageal cancer, bladder cancer, skin cancer, and head and neck cancer.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and the control sample.

In some embodiments the level of at least one of the biomarkers of the invention, which are myc transcription factor markers or surrogate markers thereof, such as translation addicted markers as defined herein, e.g. one or more of EIF4EBP1, EIF4EBP2, L-Myc, N-Myc and c-Myc, or combinations thereof, is determined in the test sample and a molecule other than the marker molecule, which can be used for determining a reference or control value is determined in the control sample. Molecules, whose level can be used as reference or control values include any housekeeping gene (or gene sets) or gene products therefrom, which refers to a gene (or gene sets) involved in basic functions needed for maintenance of the cell, and which are transcribed at a relatively constant level and are thus used to normalize gene levels that vary across different samples, e.g. GAPDH, β-glucuronidase (GUSB), actin, ubiquitin, tubulin, and the like.

In some embodiments, the control sample is obtained from a healthy subject. In some embodiments, the control sample is a non-cancerous biological sample obtained from the cancer patient, i.e. from a tissue or body part not affected by the cancer, such as from a tissue matched healthy sample. In some embodiments, the control sample is obtained from the cancer patient during a treatment with a GSPT1 negative modulator, and is a cancerous biological sample taken prior to treatment or at an earlier time point during the treatment. In some embodiments, the control sample is obtained from a different cancer patient, i.e. a cancer patient other than the cancer patient of which the cancerous sample has been obtained, which has previously been determined to be a responder or a non-responder.

In some embodiments, the method to determine or assess the responsiveness of a cancer patient to a treatment with a GSPT1 negative modulator is carried out before the cancer patient is subjected to the treatment with a GSPT1 negative modulator. In some embodiments, the method is carried out during the cancer patient is subjected to the treatment with a GSPT1 negative modulator. In some embodiments, the method is carried out after the cancer patient has been subjected to the treatment with a GSPT1 negative modulator.

In some embodiments, the biomarker used according to the disclosure is EIF4EBP1. In some embodiments, the biomarker used according to the disclosure is EIF4EBP1 and the cancer, e.g., myc-driven cancer to be treated is breast cancer or SCLC. In some embodiments, the biomarker used according to the disclosure is EIF4EBP1 and the cancer, e.g., myc-driven cancer, to be treated is breast cancer.

Thus, in some embodiments, the disclosure provides a use of EIF4EBP1 to evaluate the responsiveness of a cancer patient, such as a patient suffering from breast cancer or SCLC, to a treatment with a GSPT1 negative modulator, comprising the steps of
(i) obtaining a cancerous sample from the patient,
(ii) determining the level of EIF4EBP1 expression or phosphorylation in the cancerous sample,
(iii) comparing the level of EIF4EBP1 expression or phosphorylation determined in step (ii) with the level of one or more reference markers determined in a control sample, and
(iv) identifying the patient as having an increased responsiveness to the treatment if the level of EIF4EBP1 expression or phosphorylation in the cancerous sample is elevated as compared to the level of the one or more reference markers in the control sample or identifying the patient as having a reduced responsiveness to the treatment if the level of EIF4EBP1 expression or phosphorylation in the cancerous sample is equal to or lower as compared to the level of the one or more reference markers in the control sample.

In some embodiments, the biomarker used according to the disclosure is EIF4EBP2. In some embodiments, the biomarker used according to the disclosure is EIF4EBP2 and the cancer, e.g., myc-driven cancer, to be treated is breast cancer or SCLC. In some embodiments, the biomarker used according to the disclosure is EIF4EBP2 and the cancer, e.g., myc-driven cancer, to be treated is breast cancer.

Thus, in some embodiments, the disclosure provides a use of EIF4EBP2 to evaluate the responsiveness of a cancer patient, such as a patient suffering from breast cancer or SCLC, to a treatment with a GSPT1 negative modulator, comprising the steps of
(i) obtaining a cancerous sample from the patient,
(ii) determining the level of EIF4EBP2 expression or phosphorylation in the cancerous sample,
(iii) comparing the level of EIF4EBP2 expression or phosphorylation determined in step (ii) with the level of one or more reference markers determined in a sample, and
(iv) identifying the patient as having an increased responsiveness to the treatment if the level of EIF4EBP2 expression or phosphorylation in the cancerous sample is elevated as compared to the level of the one or more reference markers in the control sample or identifying the patient as having a reduced responsiveness to the treatment if the level of EIF4EBP2 expression or phosphorylation in the cancerous sample is equal to or lower as compared to the level of the one or more reference markers in the control sample.

In some embodiments, the biomarker used according to the disclosure is L-Myc. In some embodiments, the biomarker used according to the disclosure is L-Myc and the cancer, e.g., myc-driven cancer to be treated is breast cancer or SCLC. In some embodiments, the biomarker used according to the disclosure is EIF4EBP1 and the cancer, e.g., myc-driven cancer, to be treated is SCLC.

Thus, in some embodiments, the disclosure provides a use of L-Myc to evaluate the responsiveness of a cancer patient, such as a patient suffering from breast cancer or SCLC, to a treatment with a GSPT1 negative modulator, comprising the steps of
(i) obtaining a cancerous sample from the patient,
(ii) determining the level of L-Myc in the cancerous sample,
(iii) comparing the level of L-Myc determined in step (ii) with the level of one or more reference markers determined in a control sample, and
(iv) identifying the patient as having an increased responsiveness to the treatment if the level of L-Myc in the cancerous sample is elevated as compared to the level of the one or more reference markers in the control sample or identifying the patient as having a reduced responsiveness to the treatment if the level of L-Myc in the cancerous sample is equal to or lower as compared to the level of the one or more reference markers in the control sample.

In some embodiments, a combination of biomarkers is used according to the disclosure, which include EIF4EBP1 and L-Myc. In some embodiments, a combination of biomarkers is used according to the disclosure, which include EIF4EBP1 and L-Myc and the cancer, e.g., myc-driven cancer, to be treated is breast cancer or SCLC.

In some embodiments, a combination of biomarkers is used according to the disclosure, which include EIF4EBP2 and L-Myc. In some embodiments, a combination of biomarkers is used according to the disclosure, which include EIF4EBP2 and L-Myc and the cancer, e.g., myc-driven cancer, to be treated is breast cancer or SCLC.

In some embodiments, a combination of biomarkers is used according to the disclosure, which include EIF4EBP1 and EIF4EBP2. In some embodiments, a combination of biomarkers is used according to the disclosure, which include EIF4EBP1 and EIF4EBP2 and the cancer, e.g., myc-driven cancer, to be treated is breast cancer or SCLC.

In some embodiments, a combination of biomarkers is used according to the disclosure, which include EIF4EBP1, EIF4EBP2 and L-Myc. In some embodiments, a combination of biomarkers is used according to the disclosure, which include EIF4EBP1, EIF4EBP2 and L-Myc and the cancer, e.g., myc-driven cancer, to be treated is breast cancer or SCLC.

Thus, in some embodiments the disclosure provides a use of EIF4EBP1 and/or EIF4EBP2 and/or L-Myc to evaluate the responsiveness of a cancer patient, such as a patient suffering from breast cancer or SCLC, to a treatment with a GSPT1 negative modulator, comprising the steps of
(i) obtaining a cancerous sample from the patient,
(ii) determining the level of EIF4EBP1 expression or phosphorylation and/or EIF4EBP2 and/or L-Myc in the cancerous sample,
(iii) comparing the level of the EIF4EBP1 and/or EIF4EBP2 and/or L-Myc determined in step (ii) with the level of one or more reference markers determined in a control sample, and
(iv) identifying the patient as having an increased responsiveness to the treatment if the level of EIF4EBP1 expression or phosphorylation and/or EIF4EBP2 and/or L-Myc in the cancerous sample is elevated as compared to the level of the one or more reference markers in the control sample or identifying the patient as having a reduced responsiveness to the treatment if the level of EIF4EBP1 expression or phosphorylation and/or EIF4EBP2 and/or L-Myc in the cancerous sample is equal to or lower as compared to the level of the one or more reference markers in the control sample.

In some embodiments, the biomarker used according to the disclosure is N-Myc. In some embodiments, the biomarker used according to the disclosure is N-Myc and the cancer, e.g., myc-driven cancer to be treated is NSCLC.

Thus, in some embodiments the disclosure provides a use of N-Myc to evaluate the responsiveness of a cancer patient, such as a patient suffering from NSCLC, to a treatment with a GSPT1 negative modulator, comprising the steps of
(i) obtaining a cancerous sample from the patient,
(ii) determining the level of N-Myc thereof in the cancerous sample,
(iii) comparing the level of N-Myc determined in step (ii) with the level of one or more reference markers determined in a control sample, and
(iv) identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

In some embodiments, the biomarker used according to the disclosure is N-Myc. In some embodiments, the biomarker used according to the disclosure is N-Myc and the cancer, e.g., myc-driven cancer to be treated is a neuroendocrine cancer, for example, neuroendocrine prostate cancer (for example, NEPC (castration-resistant neuroendocrine prostate cancer)) and lung neuroendocrine tumors (Lu-NETs).

Thus, in some embodiments the disclosure provides a use of N-Myc to evaluate the responsiveness of a cancer patient, such as a patient suffering from a neuroendocrine cancer, for example, neuroendocrine prostate cancer (for example, NEPC (castration-resistant neuroendocrine prostate cancer)) and lung neuroendocrine tumors (Lu-NETs), to a treatment with a GSPT1 negative modulator, comprising the steps of
(i) obtaining a cancerous sample from the patient,
(ii) determining the level of N-Myc thereof in the cancerous sample,
(iii) comparing the level of N-Myc determined in step (ii) with the level of one or more reference markers determined in a control sample, and
identifying the patient as having an increased responsiveness to the treatment if the level of N-Myc in the cancerous sample is elevated as compared to the level of the one or more reference markers in the control sample or identifying the patient as having a reduced responsiveness to the treatment if the level of N-Myc in the cancerous sample is equal to or lower as compared to the level of the one or more reference markers in the control sample.

The level of the one or more biomarkers is determined by measuring the expression at the nucleic acid level or at the polypeptide level, i.e. by measuring the level of the biomarker mRNA or the biomarker protein or a derivative form thereof, such as a form after posttranslational modifications, in particular the phosphorylated form.

The method according to the disclosure comprises a (quantitative) determination of the level in a biological sample (e.g. sample of the tumor tissue or tumor cells) of one or more biomarkers selected from a myc transcription factor marker or surrogate marker thereof, such as a translation addicted marker as defined herein, e.g. one or more of EIF4EBP1, EIF4EBP2, L-Myc, N-Myc and c-Myc, or combinations thereof for the assessment of sensitivity of a cancer (e.g. a tumor or a tumor cell) to a treatment with a GSPT1 negative modulator. An increased expression compared to a control sample indicates an increased sensitivity of a cancer (e.g. a tumor or a tumor cell) and a decreased expression compared to a control sample indicates a decreased sensitivity of a cancer (e.g. a tumor or a tumor cell).

A reference or control value can be obtained from a control sample and may be determined from any suitable reference or control tissue sample by measuring the extent of expression of the selected biomarker molecule or another molecule as a control. In one embodiment the extent of expression of the biomarker molecule or a different molecule is determined in a tissue sample obtained from the same patient but from the surrounding normal tissue (not affected by the cancer). In one embodiment the extent of expression of the biomarker molecule or a different molecule is determined in a tissue sample obtained from a patient diagnosed with the same cancer but responsive or non-responsive to the treatment with a GSPT1-modulator.

The reference or control value can be a level of biomarker (e.g., a myc transcription factor marker or surrogate marker thereof, such as a translation addicted marker as defined herein, e.g. one or more of EIF4EBP1, EIF4EBP2, L-Myc, N-Myc and c-Myc, or combinations thereof) expression that was previously determined to be a value above which a tumor will have increased sensitivity to a GSPT1 negative modulator.

Biomarkers of the disclosure (and the extent of (over- or under-) expression of a biomarker) may be analysed e.g. on the nucleic acid level and/or the polypeptide level according to the methods and standard procedures described herein and known in the art to quantify levels and identify a profile and any alterations thereof. Any method having adequate specificity and sensitivity known in the art is suitable. In some embodiments a determination is carried out at the nucleic acid level, e.g. by measuring DNA amplification, RNA, DNA hypo- or hyper-methylation. Quantitative determinations of expression at the nucleic acid level can include, for example, hybridization with labelled biomarker-specific probes, nucleic acid amplification reactions, gene chip hybridizations, transcript sequencing, and the like as detailed herein. Exemplary determination methods include e.g. quantitative PCR (qPCR) or realtime PCR (rtPCR).

Typical analysis methods include e.g. copy number detection of a biomarker nucleic acid, which are well known in the art, for example various hybridization-based assays, such as traditional “direct probe” methods, e.g. Southern blots, in situ hybridization (e.g, FISH and FISH plus SKY) methods, and “comparative probe” methods, such as comparative genomic hybridization (CGH), e.g, cDNA-based or oligonucleotide-based CGH. The methods can be used in a wide variety of formats including substrate (e.g. membrane or glass) bound methods or array-based approaches.

In some embodiments the biomarker gene copy number in a sample is determined using a Southern Blot, in which genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g, a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g, a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g, a non-amplified portion of the same or related cell tissue, organ, etc.) provides an estimate of the relative copy number of the target nucleic acid.

In some embodiments the copy number is determined by in situ hybridization (e.g, Angerer (1987) Meth. Enzymol 152: 649). Generally, in situ hybridization comprises the steps of: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labelled probes specific to the nucleic acid sequence encoding the protein. The targets (e.g., cells) are then typically washed at a predetermined stringency or at an increasing stringency until an appropriate signal to noise ratio is obtained. The probes are typically labelled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

In some embodiments the copy number is determined by comparative genomic hybridization. In general, genomic DNA is isolated from normal reference cells, as well as from test cells (e.g, tumor cells) and amplified, if necessary. The two nucleic acids are differentially labelled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labelled DNA sequences are then rendered in a visualizable form, if necessary.

An increasing or decreasing copy number of chromosomal regions in test cells can also be identified by detecting regions where the ratio of signal from the two DNAs is altered. For example, regions with a decreased copy number in the test cells will show relatively lower signal from the test DNA than the reference compared to other regions of the genome. Regions with an increased copy number in the test cells will show relatively higher signal from the test DNA. Where there are chromosomal deletions or multiplications, differences in the ratio of the signals from the two labels will be detected and the ratio will provide a measure of the copy number.

In some embodiments array CGH (aCGH) is used, wherein the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labelling (as opposed to labelling the control and the possible sample with two different dyes and mixing them prior to hybridization, which will yield a ratio due to competitive hybridization of probes on the arrays). In single colour CGH, the control is labelled and hybridized to one array and absolute signals are read, and the possible sample is labelled and hybridized to a second array (with identical content) and absolute signals are read. Copy number difference is calculated based on absolute signals from the two arrays. Methods of preparing immobilized chromosomes or arrays and performing comparative genomic hybridization are well known in the art is used.

In some embodiments the copy number is determined by amplification-based assays, wherein the nucleic acid sequences act as a template in an amplification reaction (e.g, Polymerase Chain Reaction (PCR). In a quantitative amplification, the amount of amplification product will be proportional to the amount of template in the original sample. Comparison to appropriate controls, e.g. healthy tissue, provides a measure of the copy number. Quantitative amplification methods are well known in the art and include e.g. quantitative PCR, which involves simultaneously co-amplifying a known quantity of a control sequence using the same primers (providing an internal standard to calibrate the PCR reaction). Detailed protocols for quantitative PCR may be found e.g. in Innis, et al. (1990) PCR Protocols, A Guide to Methods and Applications, Academic Press, Inc. N.Y.). Known nucleic acid sequence allows to routinely select primers to amplify any portion of the gene. Other variations that may be included is fluorogenic quantitative PCR, wherein quantitation is based on amount of fluorescence signals. Other suitable amplification methods include, e.g., ligase chain reaction, transcription amplification, self-sustained sequence replication, dot PCR, and linker adapter PCR, etc.

Expression of a biomarker may further be monitored by detecting mRNA levels, protein levels (or protein activity), which can be measured using standard techniques known in the art and involve e.g. quantification of the level of gene expression (e.g. genomic DNA, cDNA, mRNA, protein, or enzyme activity). In some embodiment, one or more cells from the subject to be tested are obtained and RNA is isolated from the cells, which includes cancer cells as well as healthy cells (as a control).

In some embodiments, a single cell can be isolated from a tissue sample by laser capture microdissection (LCM) known in the art. In some embodiments, cells obtained from a subject are cultured in vitro (using methods well known in the art) to obtain larger cell populations of which RNA can be extracted (e.g. by guanidium thiocyanate lysis followed by CsCl centrifugation).

In some embodiments, the RNA population is enriched in marker sequences, e.g., by primer-specific cDNA synthesis, or multiple rounds of linear amplification based on cDNA synthesis and template-directed in vitro transcription. Amplification processes, such as RT-PCR, strand displacement amplification, target mediated amplification, ligase chain reaction, self-sustained sequence replication, transcription amplification, may be used to amplify the mRNA, such that a signal is detectable or detection is enhanced.

Known methods in the art for determining absolute and relative levels of gene expression, include Northern analysis, RNase protection assays (RPA), microarrays and PCR-based techniques, such as quantitative PCR and differential display PCR, NGS and Nanostring platforms. One of skill will appreciate that other nucleic acid analysis methods can be used. For example, Northern blotting involves running a preparation of RNA on a denaturing agarose gel, and transferring it to a suitable support, such as activated cellulose, nitrocellulose or glass or nylon membranes. Radiolabelled cDNA or RNA is then hybridized to the preparation, washed and analyzed by autoradiography. In situ hybridization visualization may also be employed, wherein a radioactively labelled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used. NGS (next-generation sequencing) includes a variety of high-throughput sequencing technologies that may be applied to measure expression levels of nucleic acids (DNA, e.g. copy number; DNA mutations, e.g. of KRAS; RNA expression). Nanostring platforms (e.g. NanoString's Counter® system or Digital Spatial Profiling (DSP) platform may be used for nucleic acid or protein detection.

Alternatively, mRNA expression can be detected on a DNA array, chip or a microarray. Labelled nucleic acids of a test sample obtained from a subject may be hybridized to a solid surface comprising biomarker DNA. Positive hybridization signal is obtained with the sample containing biomarker transcripts. To monitor mRNA levels, for example, mRNA is extracted from the biological sample to be tested, reverse transcribed, and fluorescently-labelled cDNA probes are generated. The microarrays capable of hybridizing to marker cDNA are then probed with the labelled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In some embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker (wherein the term “stringent conditions” as used herein means hybridization will occur only if there is at least 95% identity in nucleotide sequences). In some embodiment, hybridization under stringent conditions occurs when there is at least 97% identity between the sequences.

Alternatively or additionally to the above methods, biomarkers may be detected and/or quantified on the (expressed) polypeptide level using methods well known in the art and include immunodiffusion, Immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

In some embodiments, ELISA and RIA procedures may be conducted such that a desired biomarker protein standard is labelled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labelled anti-biomarker protein antibody is allowed to react with the system, and radioactivity or the enzyme assayed (ELISA-sandwich assay). Other conventional methods may also be employed as suitable.

In some embodiments, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein. Enzymatic and radiolabelling methods are well known in the art and include covalent linking of the enzyme to the antigen or the antibody in question, such as by glutaraldehyde, specifically so as not to adversely affect the interaction of the enzyme with its substrate.

In some embodiments, a biomarker protein may be detected by Western blotting, wherein a suitably treated sample is run on an SDS-PAGE gel before being transferred to a solid support, such as a nitrocellulose filter. Anti-biomarker protein antibodies (unlabelled) are then brought into contact with the support and assayed by a secondary immunological reagent, such as labelled protein A or anti-immunoglobulin (suitable labels including ¹²⁵I, horseradish peroxidase and alkaline phosphatase).

In some embodiments, immunohistochemistry may be used to detect expression of biomarker protein, e.g., in a biopsy sample. A suitable antibody is brought into contact with, for example, a thin layer of cells, washed, and then contacted with a second, labelled antibody. Labelling may be by fluorescent markers, enzymes, such as peroxidase, avidin, or radiolabelling. The assay is scored visually, using microscopy. In some embodiments, anti-biomarker protein antibodies may be used for imaging purposes, for example, to detect the presence of biomarker protein in cells and tissues of a subject. Antibodies (commercially available or synthetic or engineered antibodies prepared according to methods known in the art) that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10⁶M, 10⁷M, 10⁸M, 10⁹M, 10¹⁰M, 10¹¹M, 10¹²M. Such antibodies and derivatives thereof include polyclonal or monoclonal antibodies, chimeric, human, humanized, primatized (CDR-grafted), veneered or single-chain antibodies as well as functional fragments, i.e., biomarker protein binding fragments, of antibodies.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

In some embodiments, the GSPT1 modulator used in the methods of the disclosure is a compound or a pharmaceutically acceptable salt or stereoisomer thereof of formula I:

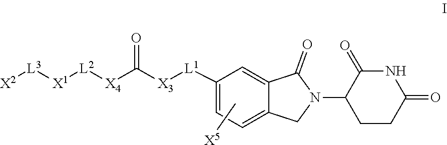

X¹ is linear or branched C₁₋₆ alkyl, C₃₋₈ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy, C₁₋₆ alkylhydroxy C₁₋₄alkylhydroxy, —CH₂F, —N(H)C(O)—O—C₁₋₆alkyl, or C(OH)(CF₃);
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen

Unless specified otherwise the following general definitions apply to all compounds of the disclosure according to the description.

The term “compound of the disclosure” as used herein, refers to compounds represented by formulae I to IV (including a pharmaceutically acceptable salt or stereoisomer thereof) and any of the specific examples disclosed herein.

It is understood that “independently of each other” means that when a group is occurring more than one time in any compound, its definition on each occurrence is independent from any other occurrence. It is further understood that a dashed line (or a wave being transverse to a bond) or a solid line without attachment, such as —C₁₋₄ alkyl, depicts the site of attachment of a residue (i.e. a partial formula). It is further understood that the abbreviations “C” and “N” are representative for all possible degrees of saturation, which typically do not result in radicals, nitrenes or carbenes, i.e. N includes —NH— and —N═, C includes —CH₂— and ═CH—. In addition, “C” as an atom in an aromatic or heteroaromatic ring which has a substituent R^{x} at any suitable position, includes ═CH— as well as ═CR^{x}—.

Based on the definitions given throughout the application the skilled person knows which combinations are synthetically feasible and realistic, e.g. typically combinations of groups leading to some heteroatoms directly linked to each other, e.g. —O—O—, are not contemplated, however synthetically feasible combinations, such as —S—N═ in aisothiazole are contemplated.

The term “saturated” in reference to ring systems refers to a ring having no double or triple bonds. The term “partially unsaturated” in reference to ring systems refers to a ring that includes at least one double or triple bond, but does not include aromatic systems.

The term “aromatic” refers to monocyclic or multicyclic (e.g. bicyclic) ring systems, which show some or complete conjugation or delocalization of their electrons. Aromatic monocyclic rings, such as aryl or heteroaryl rings as defined herein, include phenyl, pyridinyl, furyl and the like. Aromatic multicyclic rings, such as aryl or heteroaryl rings as defined herein, refer to ring systems, wherein at least one ring is an aromatic ring, and thus include (i) aromatic ring systems, wherein an aromatic ring is fused to one or more aromatic rings, such as in e.g. naphthyl, indolyl, benzimidazolyl, and the like (also referred to as fully aromatic ring systems), and (ii) aromatic ring systems, wherein an aromatic ring is fused to one or more non-aromatic rings, such as in e.g. indanyl, indenyl, phthalimidyl, naphthimidyl, phenanthridinyl, tetrahydronaphthyl, 1,4-dihydronapthyl, and the like (also referred to as partially aromatic ring systems).

The term “non-aromatic” refers to (i) fully saturated rings such as monocyclic rings, e.g. cyclohexyl, and bicyclic rings, e.g. tetrahydronaphthyl, and (ii) partially unsaturated rings such as monocyclic rings, e.g. cyclohexenyl, and bicyclic rings, e.g. 1,4-dihydronapthyl.

The term “C₆₋₁₀ aryl” refers to a fully or partially aromatic ring system having 6, 7, 8, 9, 10 ring atoms and includes monocycles and fused bicycles. Examples of fully aromatic C₆₋₁₀ aryl include e.g. phenyl, indenyl, naphthyl. Examples of partially aromatic C₆₋₁₀ aryl include e.g. 2,3-dihydroindenyl, 1, 2, 3, 4-tetrahydronaphthyl. In some embodiments for group X¹ C₆₋₁₀ aryl is phenyl, 2,3-dihydroindenyl. In some embodiments for group X² C₆₋₁₀ aryl is phenyl. The term “—C₁₋₆ alkyl-C₆₋₁₀ aryl” refers to a C₆₋₁₀ aryl which is linked through a C₁₋₆ alkyl group as defined herein. The term “—C₁₋₆ alkoxy-C₆₋₁₀ aryl” refers to a C₆₋₁₀ aryl which is linked through a C₁₋₆ alkoxy group as defined herein. The term “—O—C₆₋₁₀ aryl” or “C₆₋₁₀ aryloxy” refers to a C₆₋₁₀ aryl which is linked through a —O— group. The C₆₋₁₀ aryl group may be unsubstituted or substituted with C₁₋₄ alkyl, such as methyl, ethyl, C₁₋₄ alkoxy, such as methoxy, ethoxy, halogen, such as F, Cl, or Br, such as F or Cl.

The term “5-10 membered heteroaryl” refers to a fully or partially aromatic ring system in form of monocycles or fused bicycles having 5, 6, 7, 8, 9, 10 ring atoms selected from C, N, O, and S, such as C, N, and O, or C, N, and S, with the number of N atoms being e.g. 0, 1, 2 or 3 and the number of O and S atoms each being 0, 1 or 2. In some embodiments a 5-10 membered heteroaryl refers to a fully aromatic ring system having 5, 6, 7, 8, 9, 10, such as 5 or 6, e.g. 6 ring atoms selected from C and N, with the number of N atoms being 1, 2 or 3, such as 1 or 2. In some embodiments a 5-10 membered heteroaryl refers to a fully aromatic ring system having 6 ring atoms selected from C and N, with the number of N atoms being 1 or 2. In other embodiments a 5-10 membered heteroaryl refers to a partially aromatic ring system having 9 or 10 ring atoms selected from C, N and O, with the number of O atoms being 1, 2 or 3, such as 1 or 2, and the number of N atoms being 1 or 2, such as 1. In some embodiments, examples of “5-10 membered heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, indolyl, quinazolinyl, oxazolinyl, isoxazolinyl, indazolinyl, isothiazolyl, 1,3-benzodioxolyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, benzodihydropyrane, 1,2,3,4-tetrahydronaphthyl, 2,3-dihydroindenyl and the like. In some embodiments, examples of “5-10 membered heteroaryl” include 6-membered heteroaryl, such as pyridinyl, pyrimidinyl, pyridazinyl, pyrazinyl, 9-membered heteroaryl, such as 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, and 10-membered heteroaryl, such as benzodihydropyrane (chromane), dihydropyrano-pyridine. The term “—C₁₋₆ alkyl 5-10 membered heteroaryl” refers to a 5-10 membered heteroaryl, which is linked through a C₁₋₆ alkyl group as defined herein to its neighbouring group. The term “—C₁₋₆ alkoxy 5-10 membered heteroaryl” refers to a 5-10 membered heteroaryl, which is linked through a C₁₋₆ alkoxy group as defined herein to its neighbouring group. The term “—O-5-10 membered heteroaryl” refers to a 5-10 membered heteroaryl, which is linked through a —O— group to its neighbouring group. The term “—O—C₆₋₁₀ aryl” refers to a C₆₋₁₀ aryl which is linked through a —O— group. The 5-10 membered heteroaryl group may be unsubstituted or substituted with C₁₋₄ alkyl, such as methyl, ethyl, C₁₋₄ alkoxy, such as methoxy, ethoxy, halogen, such as F, Cl or Br, e.g. F or Cl.

The term “C₃₋₆ cycloalkyl” refers to a non-aromatic, i.e. saturated or partially unsaturated alkyl ring system containing 3, 4, 5 or 6 carbon atoms, such as cyclopropyl, cyclobutyl, cyclopentyl, cyclohexyl, unsubstituted or substituted by e.g. one or more of C₁₋₄ alkyl, such as methyl and halogen, such as F.

The term “4-8 membered heterocycloalkyl” refers to a non-aromatic, i.e. saturated or partially unsaturated ring system having 4, 5, 6, 7 or 8 ring atoms (of which at least one is a heteroatom), which ring atoms are selected from C, N, O, and S, such as C, N, and O, the number of N atoms being 0, 1, or 2 and the number of 0 and S atoms each being 0, 1, or 2. In some embodiments the term “4-8 membered heterocycloalkyl” comprises saturated or partially unsaturated monocycles, fused bicycles, bridged bicycles or spirobicycles. In some embodiments the term “4-8 membered heterocycloalkyl” comprises fully saturated or partially unsaturated monocycles and bridged bicycles. Examples of 4-8 membered heterocycloalkyl groups include azetidinyl, oxetanyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, and the like. Examples of 5-6 membered heterocycloalkyl groups include pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, 2-oxa-5-azabicyclo[2.2.1]heptan-5-yl, 1,4-diazabicyclo[3.2.1]octan-4-yl, 3-methyl-3-azabicyclo[3.1.0]hexan-1-yl, 8-oxa-3-azabicyclo[3.2.1]octan-3-yl and the like. The 4-8 membered heterocycloalkyl group may be unsubstituted or substituted with C₁₋₄ alkyl, such as methyl, ethyl, C₁₋₄ alkoxy, such as methoxy, ethoxy, halogen, such as F, Cl or Br, e.g. F or Cl.

In some embodiments, the term 4-8 membered heterocycloalkyl includes 5-membered heterocycloalkyl having 1 or 2 N-atoms, such as pyrrolidinyl, 6-membered heterocycloalkyl having N and O-atoms, such as morpholinyl, piperidinyl, piperazyinyl, dioxanyl, 7-membered heterocycloalkyl having N and O-atoms, such as 1 N- and 1 O-atom, such as 2-oxa-5-azabicyclo[2.2.1]heptan-5-yl, 1,4-diazabicyclo[3.2.1]octan-4-yl, 3-methyl-3-azabicyclo[3.1.0]hexan-1-yl; 8-membered heterocycloalkyl having N and O-atoms, such as 1 N- and 1 O-atom, such as 8-oxa-3-azabicyclo[3.2.1]octan-3-yl. The term “C₁₋₄ alkyl 4-8 membered heterocycloalkyl” refers to an alkyl as defined below with 1 to 4 carbon atoms, which is bound to a 4-8 membered heterocycloalkyl as defined above. Preferably, the C₁₋₄ alkyl may be C₁, resulting in —(CH₂)-(4-8 membered heterocycloalkyl) or C₂, resulting in —(CH₂)₂-(4-8 membered heterocycloalkyl) or C₃, resulting in —(CH₂)₃-(4-8 membered heterocycloalkyl). Examples include —(CH₂)-morpholinyl, —(CH₂)₂-morpholinyl, —(CH₂)₃-morpholinyl, —(CH₂)₄-morpholinyl, —(CH₂)-piperazinyl, —(CH₂)₂—N-methyl-piperazinyl, —(CH₂)₃-piperazinyl or —(CH₂)₄-piperazinyl. The term “C₁₋₄ alkoxy 4-8 membered heterocycloalkyl” refers to a 4-7 membered heterocycloalkyl as described above, which is linked via a C₁₋₄ alkoxy group to its neighbouring group. Preferably, the C₁₋₄ alkoxy may be C₁, resulting in —(O—CH₂)-(4-8 membered heterocycloalkyl) or C₂, resulting in —(O—CH₂)₂-(4-8 membered heterocycloalkyl) or C₃, resulting in —(O—CH₂)₃-(4-8 membered heterocycloalkyl). Examples include —(O—CH₂)—(N-morpholinyl), —(O—CH₂)₂—(N-morpholinyl). The term “—O-(4-8 membered heterocycloalkyl)” refers to a 4-8 membered heterocycloalkyl as described above, which is linked via a —O-group to its neighbouring group. Examples include —O— morpholinyl, —O-piperazinyl, —O-pyrrolidinyl and the like. The term “—O(CO)—C₁₋₄ alkyl 4-7 membered heterocycloalkyl” refers to a 4-8 membered heterocycloalkyl as described above, which is linked via a —O(CO)—C₁₋₄ alkyl group to its neighbouring group. Preferably, the “—O(CO)—C₁₋₄ alkyl may be C₁, resulting in —(O(CO)—CH₂)-(4-8 membered heterocycloalkyl) or C₂, resulting in —(O(CO)—CH₂)₂-(4-8 membered heterocycloalkyl) or C₃, resulting in —(O(CO)—CH₂)₃-(4-8 membered heterocycloalkyl). Examples include —(O(CO)—CH₂)—(N-morpholinyl) or —(O(CO)—CH₂—CH₂)—(N-morpholinyl). The term “halogen” or “hal” as used herein may be fluoro, chloro, bromo or iodo preferably fluoro, chloro or bromo, more preferably fluoro or chloro.

The term “halogen” or “hal” as used herein may be fluoro, chloro, bromo or iodo such as fluoro, chloro or bromo, e.g. fluoro or chloro.

The term “C₁₋₄ alkyl” and “C₁₋₆alkyl” refer to a fully saturated branched or unbranched hydrocarbon moiety having 1, 2, 3 or 4 and 1, 2, 3, 4, 5 or 6 carbon atoms, respectively. Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, iso-hexyl or neohexyl.

The term “C₁₋₆ heteroalkyl” refers to an alkyl as defined with 1, 2, 3, 4, 5 or 6 carbon atoms in which at least one carbon atom is replaced with a heteroatom, such as N, O, or S, e.g. N, O. It is understood that the heteroatom may further be substituted with one or two C₁₋₆ alkyl. Examples include —(CH₂)₂—O-Me, —(CH₂)₃—O-Me, —(CH₂)₂—O—CH₂Me, —(CH₂)₂—NMe₂, —(CH₂)—NMe₂, —(CH₂)₂—NEt₂, —(CH₂)—NEt₂ and the like.

The term “C₁₋₄alkylamino” refers to a fully saturated branched or unbranched C₁₋₄ alkyl, which is substituted with at least one, such as only one, amino group, alkylamino group or dialkylamino group, such as NH₂, HN(C₁₋₄alkyl) or N(C₁₋₄alkyl)₂. Thus, a C₁₋₄alkylamino refers to C₁₋₄alkylamino, C₁₋₄alkyl-(C₁₋₄alkyl)amino, C₁₋₄alkyl-(C₁₋₄dialkyl)amino. Examples include but are not limited to methylaminomethyl, dimethylamonimethyl, aminomethyl, dimethylaminoethyl, aminoethyl, methylaminoethyl, n-propylamino, iso-propylamino, n-butylamino, sec-butylamino, iso-butylamino, tert-butylamino.

The term “C₁₋₄ alkoxy” refers to an unsubstituted or substituted alkyl chain linked to the remainder of the molecule through an oxygen atom, and in particular to methoxy, ethoxy, n-propoxy, iso-propoxy, n-butoxy, iso-butoxy, and t-butoxy. The term “C₁₋₄ alkyl-C₁₋₄ alkoxy” refers to a C₁₋₄ alkyl group functionalized with a C₁₋₄ alkoxy group, such as e.g. —CH₂—O—CH₃, —(CH₂)₂—O—CH₃, —(CH₂)₃—O—CH₃, —(CH₂)₄—O—CH₃, —CH₂—O—CH₂—CH₃, —CH₂—O—(CH₂)₂—CH₃, —CH₂—O—(CH₂)₃—CH₃, and branched isomers thereof.

In some embodiments of a compound of formula I, —X⁴—CO—X³— is —NH—CO—NH—. In some embodiments of a compound of formula I, —X⁴—CO—X³— is —NH—CO—O—. In some embodiments of a compound of formula I, —X⁴—CO—X³— is —CH₂—CO—NH—. In some embodiments of a compound of formula I, —X⁴—CO—X³— is —CH₂—CO—O—.

In some embodiments of a compound of formula I, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, —NH₂, C₁₋₄ alkylhydroxy, or C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, or C₁₋₆ alkoxy.

In some embodiments of a compound of formula I, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, —C₁₋₄ alkylhydroxy.

In some embodiments of formula I, X⁵ is in the 4-position or in the 5-position or in the 7-position of the ring.

In some embodiments of formula I, X⁵ is H.

In some embodiments of formula I, —X⁴—CO—X³— is —NH—CO—O— and X⁵ is H, C₁₋₄ alkyl, such as methyl, —C₁₋₄ alkoxy, such as —OMe, —CN, halogen, such as F, Cl, Br.

In some embodiments of a compound of formula I, L¹ is a linear or branched C₁₋₆ alkyl. In some embodiments of a compound of formula I, L¹ is linear or branched C₁₋₄ alkyl, such as —CH₂— or —CH(CH₃)—.

In some embodiments of a compound of formula I, L² is a covalent bond. In some embodiments of a compound of formula I, L² is linear or branched C₁₋₆ alkyl, such as linear or branched C₁₋₄ alkyl, e.g. —CH₂— or —CH(CH₃)—.

In some embodiments of a compound of formula I, L³ is a covalent bond. In some embodiments L³ is linear or branched C₁₋₄ alkyl. In some embodiments of a compound of formula I, L³ is —O—. In some embodiments of a compound of formula I, L³ is linear or branched C₁₋₄ alkoxy, such as —O—CH₂—, —O—CH₂—CH₂—.

In some embodiments of a compound of formula I, L¹ is —CH₂— and L² is a covalent bond. In some embodiments of a compound of formula I, L¹ is —CH₂— and L² is —CH₂—. In some embodiments of a compound of formula I, L¹ is —CH₂— and L² is —CH(CH₂)—.

In some embodiments of a compound of formula I, L¹ is —CH₂—, L² is a covalent bond and L³ is a covalent bond. In some embodiments of a compound of formula I, L¹ is —CH₂—, L² is a covalent bond and L³ is —CH₂—. In some embodiments of a compound of formula I, L¹ is —CH₂—, L² is a covalent bond and L³ is —O—. In some embodiments of a compound of formula I, L¹ is —CH₂—, L² is a covalent bond and L³ is —O—CH₂—. In some embodiments of a compound of formula I, L¹ is —CH₂—, L² is a covalent bond and L³ is —O—CH₂—CH₂—.

In some embodiments the GSPT1 modulator used in the methods of the disclosure is a compound or a pharmaceutically acceptable salt or stereoisomer thereof of formula II

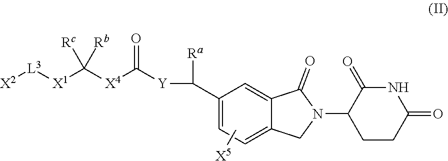

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is N or O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen;
p is 0, 1, 2.

In some embodiments of a compound of formula II, Y is NH.

In some embodiments of a compound of formula II, Y is O.

In some embodiments of a compound of formula II, R^{a} is H. In some embodiments of a compound of formula II, R^{a} is methyl.

In some embodiments of a compound of formula II, R^{b} and R^{c} are H. In some embodiments of a compound of formula II, R^{b} is linear or branched C₁₋₄ alkyl, such as methyl and R^{c} is H.

In some embodiments of formula II, X⁵ is in the 4-position or in the 5-position or in the 7-position of the ring.

In some embodiments of formula II, X⁵ is H.

In some embodiments of formula II, —X⁴—CO—X³— is —NH—CO—O— and X⁵ is H, C₁₋₄ alkyl, such as methyl, —C₁₋₄ alkoxy, such as —OMe, —CN, halogen, such as F, Cl, Br.

In some embodiments of a compound of formula II, L³ is a covalent bond. In some embodiments of a compound of formula II, L³ is linear or branched C₁₋₄ alkyl. In some embodiments of a compound of formula II, L³ is —O—. In some embodiments of a compound of formula II, L³ is linear or branched C₁₋₄ alkoxy, such as —O—CH₂—, —O—CH₂—CH₂—, O—CH₂—CH₂—CH₂—.

In some embodiments of a compound of formula II, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, and C₁₋₄ alkylhydroxy, or C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula II, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, p is 0 and X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0 and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0 and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0 and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0 and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula II, p is 0 and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 0 and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 0 and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, p is 0, R^{a} is H and X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0, R^{a} is H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula II, p is 0, R^{a} is H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 0, R^{a} is H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 0, R^{a} is H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, p is 1, R^{b} and R^{c} are H and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} and R^{c} are H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula II, p is 1, R^{b} and R^{c} are H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 1, R^{b} and R^{c} are H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 1, R^{b} and R^{c} are H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, p is 1, R^{b} is methyl and R^{c} is H and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} is methyl and R^{c} is H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula II, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄alkoxy; or X¹ together with X⁴ forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula II, p is 1, R^{b} is methyl and R^{c} is H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 1, R^{b} is methyl and R^{c} is H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, p is 1, R^{b} is methyl and R^{c} is H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is a covalent bond and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is a covalent bond and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is a covalent bond and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is a covalent bond and X² is cyclopropyl, azetidinyl, oxetanyl, cyclobutyl, pyrrolidinyl, piperdinyl, piperazinyl, morpholinyl, 8-oxa-3-azabicyclo[3.2.1]octan-3-yl, pyridyl, wherein X² is unsubstituted or substituted with one or more of linear or branched C₁₋₆ alkyl, such as methyl, —C₁₋₄ alkoxy, such as methoxy, NH₂, NMe₂ and halogen, such as fluoro.

In some embodiments of a compound of formula II, L³ is linear or branched C₁₋₄ alkyl, such as —CH₂—, and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is linear or branched C₁₋₄ alkyl, such as —CH₂—, and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is linear or branched C₁₋₄ alkyl, such as —CH₂—, and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is linear or branched C₁₋₄ alkyl, such as —CH₂—, and X² is morpholinyl, 2-oxa-5-azabicyclo[2.2.1]heptanyl, 8-oxa-3-azabicyclo[3.2.1]octan-3-yl.

In some embodiments of a compound of formula II, L³ is —O— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is —O— and X² is cyclopropyl, pyrrolidinyl, N-methyl-pyrrolidinyl, In some embodiments of a compound of formula II, L³ is —O—CH₂— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O—CH₂— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O—CH₂— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is —O—CH₂— and X² is pyrrolidinyl, N-methyl-pyrrolidinyl.

In some embodiments of a compound of formula II, L³ is —O—CH₂—CH₂— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O—CH₂—CH₂— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula II, L³ is —O—CH₂—CH₂— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula II, L³ is —O—CH₂—CH₂— and X² is morpholinyl.

In some embodiments of a compound of formula II, X¹ is a C₆ aryl or 6-membered heteroaryl, such as a pyridine, pyridazine, pyrimidine or pyrazine. In some embodiments of a compound of formula I, X¹ is a partially aromatic 6 to 10 membered heteroaryl, such as a 5-6 or 6-6 fused ring system with a 6 membered ring being a phenyl or pyridyl group. In some embodiments of a compound of formula I, X¹ is a C₁₋₆ alkyl, C₃₋₆ cycloalkyl.

In some embodiments, the present disclosure is directed towards a compound or a pharmaceutically acceptable salt or stereoisomer thereof of formula IIIa, IIIb, or IIIc

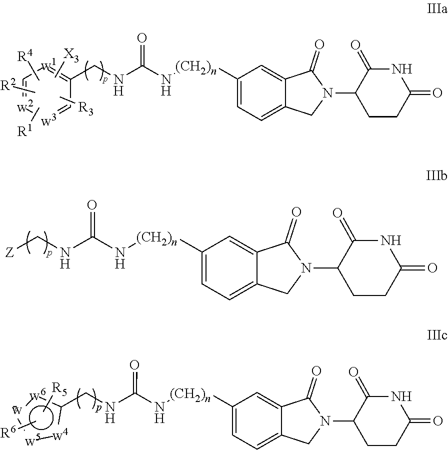

wherein
n is 1 or 2
p is 0 or 1
one of w¹, w² or w³ is selected from C and N, and the other two of w¹, w² or w³ are C;
one or two of w⁴, w⁵, w⁶, w⁷ is selected from C, O, N, NMe, NH, or S while two or three of w⁴, w⁵, w⁶ and w⁷ are C;
R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄alkylhydroxy, halogen, preferably F, Cl, Br, more preferably F or Cl; and/or two of R¹, R², R³, R⁴ form together a 5-6 membered heterocycloalkyl or a 5-6 membered heteroaryl;
R⁵, R⁶ each are independently selected from hydrogen, linear or branched C₁₋₄ alkyl, CF₃, CHF₂, halogen, preferably F, Cl, Br, more preferably F or Cl;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl;
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl.

In some embodiments of a compound of formula IIIa, IIIb, or IIIc, n is 1. In some embodiments of a compound of formula IIIa, IIIb or IIIc, p is 0. In some embodiments of a compound of formula IIIa, IIIb or IIIc, p is 1. In some embodiments of a compound of formula IIIa, IIIb or IIIc, n is 1 and p is 0 or 1. In some embodiments of a compound of formula IIIa, IIIb or IIIc, n is 1 and p is 0.

In some embodiments of a compound of formula IIIa, R¹, R², R³ are defined as above and R⁴ is hydrogen such that the aromatic ring contains 4 or 5 substituents which are not hydrogen.

In some embodiments of a compound of formula IIIa, R¹ and R² are defined as above and R³ and R⁴ each are hydrogen, such that the aromatic ring contains 3 or 4 substituents which are not hydrogen.

In some embodiments of a compound of formula IIIa, IIIb, or IIIc, R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, n is 1 and R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, preferably F, Cl, Br, more preferably F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, n is 1 and R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, p is 0 or 1 and R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, preferably F, Cl, Br, more preferably F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, p is 0 or 1 and R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, p is 0 or 1 and R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, preferably F, Cl, Br, more preferably F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, p is 0 or 1 and R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, n is 1, p is 0 or 1 and R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, preferably F, Cl, Br, more preferably F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, n is 1, p is 0 or 1 and R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa, IIIb, or IIIc, n is 1, p is 0 or 1 and R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, preferably F, Cl, Br, more preferably F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe, or 5-6 membered heterocycloalkyl.

In some embodiments of a compound of formula IIIa, IIIb, or IIIc, n is 1, p is 0 or 1 and R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄alkylhydroxy, halogen, preferably F, Cl, Br, more preferably F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIb, C₁₋₆ alkyl is selected from methyl, ethyl, n-propyl, iso-propyl, n-butyl, iso-butyl, t-butyl, n-pentyl, iso-pentyl, neopentyl, n-hexyl, iso-hexyl, and neohexyl.

In some embodiments of a compound of formula IIIb, C₃₋₆ cycloalkyl is selected from cyclopropyl, cyclobutyl, cyclopentyl, and cyclohexyl.

In some embodiments of a compound of formula IIIb, C₁₋₄ alkoxy is selected from methoxy, ethoxy, n-propoxy, iso-propoxy, n-butoxy, iso-butoxy, and t-butoxy.

In some embodiments of a compound of formula IIIb, “C₁₋₄ alkyl-C₁₋₄ alkoxy” is selected from methyl-methoxy, methyl-ethoxy, methyl-n-propoxy, methyl-iso-propoxy, methyl-n-butoxy, methyl-iso-butoxy, methyl-t-butoxy, ethyl-methoxy, ethyl-ethoxy, ethyl-n-propoxy, ethyl-iso-propoxy, ethyl-n-butoxy, ethyl-iso-butoxy, ethyl-t-butoxy, propyl-methoxy, propyl-ethoxy, propyl-n-propoxy, propyl-iso-propoxy, propyl-n-butoxy, propyl-iso-butoxy, and propyl-t-butoxy.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy and n is 1.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0 or 1.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0 or 1.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1 and p is 0 or 1.

In some embodiments of a compound of formula IIIb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1 and p is 0 or 1.

In some embodiments, the present disclosure is directed to compounds or a pharmaceutically acceptable salt or stereoisomer thereof of formula IIIa-1

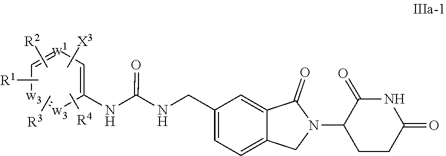

wherein
one of w¹, w² or w³ is selected from C and N, and the other two of w¹, w² or w³ are C;
R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄alkylhydroxy, halogen, preferably F, Cl, Br, more preferably F or Cl; and/or two of R¹, R², R³, R⁴ form together a 5-6 membered heterocycloalkyl or a 5-6 membered heteroaryl;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa-1, R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa-1, R¹, R², R³ are defined as above and R⁴ is hydrogen such that the aromatic ring contains 4 or 5 substituents which are not hydrogen.

In some embodiments of a compound of formula IIIa-1, R¹ and R² are defined as above and R³ and R⁴ each are hydrogen, such that the aromatic ring contains 3 or 4 substituents which are not hydrogen.

In some embodiments, the present disclosure is directed to compounds or a pharmaceutically acceptable salt or stereoisomer thereof of formula IIIa-2, IIIa-3, IIIa-4 or IIIa-5

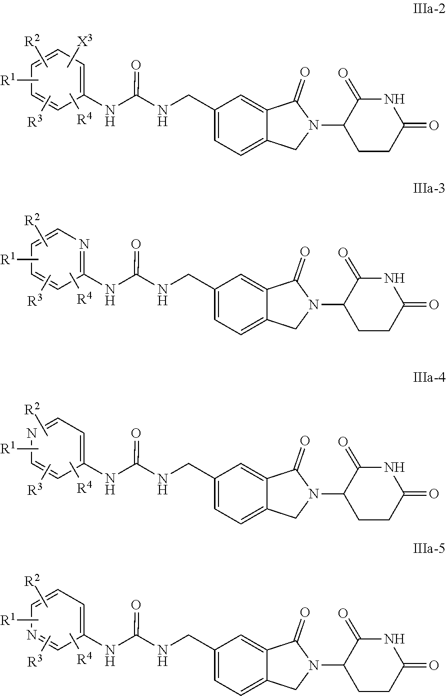

wherein
R¹, R², R³, R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄alkylhydroxy, halogen, preferably F, Cl, Br, more preferably F or Cl; and/or two of R¹, R², R³, R⁴ form together a 5-6 membered heterocycloalkyl or a 5-6 membered heteroaryl;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa-2, IIIa-3, IIIa-4 or IIIa-5 R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IIIa-2, R¹, R², R³ and R⁴ each are independently selected from hydrogen, linear or branched C₁₋₆ alkyl, linear or branched C₁₋₆ heteroalkyl, C₆ aryl, preferably phenyl, CF₃, CHF₂, —O—CHF₂, OCF₃, —CN, —CHO, —C₁₋₆alkylC(O)OH, NH₂, C₁₋₄ alkylhydroxy, halogen, preferably F, Cl, Br, more preferably F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl; while in compounds of formula IIIa-3, IIIa-4 or IIIa-5 R¹ and R² each are independently selected from hydrogen, linear or branched C₁₋₆ alkyl, and halogen, preferably Cl, while R³ and R⁴ are hydrogen.

In some embodiments the compound of formula IIIb has formula IIIb-1

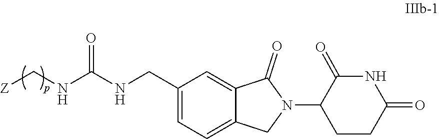

wherein
p is 0 or 1; and
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl.

In some embodiments of the compound of formula IIIb-1, C₁₋₆ alkyl is selected from methyl, ethyl, n-propyl, iso-propyl, n-butyl, iso-butyl, t-butyl, n-pentyl, iso-pentyl, neopentyl, n-hexyl, iso-hexyl, and neohexyl.

In some embodiments of the compound of formula IIIb-1, C₃₋₆ cycloalkyl is selected from cyclopropyl, cyclobutyl, cyclopentyl, and cyclohexyl.

In some embodiments of the compound of formula IIIb-1, C₁₋₄ alkoxy is selected from methoxy, ethoxy, n-propoxy, iso-propoxy, n-butoxy, iso-butoxy, and t-butoxy.

In some embodiments of the compound of formula IIIb-1, C₁₋₄ alkyl-C₁₋₄ alkoxy” is selected from methyl-methoxy, methyl-ethoxy, methyl-n-propoxy, methyl-iso-propoxy, methyl-n-butoxy, methyl-iso-butoxy, methyl-t-butoxy, ethyl-methoxy, ethyl-ethoxy, ethyl-n-propoxy, ethyl-iso-propoxy, ethyl-n-butoxy, ethyl-iso-butoxy, ethyl-t-butoxy, propyl-methoxy, propyl-ethoxy, propyl-n-propoxy, propyl-iso-propoxy, propyl-n-butoxy, propyl-iso-butoxy, and propyl-t-butoxy.

In further specific embodiments of the compound of formula IIIb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0.

In further specific embodiments of the compound of formula IIIb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 1.

In yet further specific embodiments of the compound of formula IIIb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 2.

In some embodiments, the compounds of formula IIIc are of formula IIIc-1

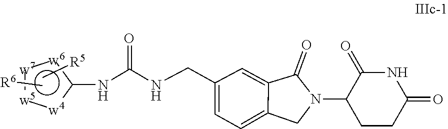

wherein
one or two of w⁴, w⁵, w⁶, w⁷ is selected from C, O, N, NMe, NH, or S while two or three of w⁴, w⁵, w⁶ and w⁷ are C;
R⁵, R⁶ each are independently selected from hydrogen, linear or branched C₁₋₄ alkyl, CF₃, CHF₂, halogen, preferably F, Cl, Br, more preferably F or Cl.

In yet specific embodiments of compounds of formula IIIc-1 or a pharmaceutically acceptable salt or stereoisomer thereof, R⁵, R⁶ each are independently selected from hydrogen, methyl, ethyl and CF₃.

In some embodiments of compounds of formula IIIc-1 or a pharmaceutically acceptable s salt or stereoisomer thereof, w⁵ is N, w⁷ is NMe, w⁶ and w⁴ are C; or w⁵ is C, w¹ is S, w⁶ and w⁴ are C; or w⁵ is C, w⁷ is NMe, w⁶ is N and w⁴ is C; or w⁵ is C, w⁷ is C, w⁶ is C and w⁴ is S; or w⁵ is C, w⁷ is C, w⁶ is N and w⁴ is N; or w⁵ is O, w⁷ is C, w⁶ is C and w⁴ is S; or w⁵ is NH, w⁷ is C, w⁶ is C and w⁴ is C; or w⁵ is C, w⁷ is S, w⁶ is C and w⁴ is N; or w⁵ is NH, w⁷ is C, w⁶ is C and w⁴ is N; or w⁵ is C, w⁷ is N, w⁶ is C and w⁴ is S; or w⁵ is NH, w⁷ is N, w⁶ is C and w⁴ is C; or w⁵ is C, w¹ is NMe, w⁶ is C and w⁴ is C; or w⁵ is N, w⁷ is C, w⁶ is C and w⁴ is S; or w⁵ is C, w is C, w⁶ is S and w⁴ is N.

In some embodiments of compounds of formula IIIc-1 or a pharmaceutically acceptable salt or stereoisomer thereof, R⁵, R⁶ each are independently selected from hydrogen, methyl, ethyl and CF₃ and w⁵ is N, w⁷ is NMe, w⁶ and w⁴ are C; or w⁵ is C, w⁷ is S, w⁶ and w⁴ are C; or w⁵ is C, w⁷ is NMe, w⁶ is N and w⁴ is C; or w⁵ is C, w⁷ is C, w⁶ is C and w⁴ is S; or w⁵ is C, w⁷ is C, w⁶ is N and w⁴ is N; or w⁵ is 0, w⁷ is C, w⁶ is C and w⁴ is S; or w⁵ is NH, w⁷ is C, w⁶ is C and w⁴ is C; or w⁵ is C, w⁷ is S, w⁶ is C and w⁴ is N; or w⁵ is NH, w⁷ is C, w⁶ is C and w⁴ is N; or w⁵ is C, w⁷ is N, w⁶ is C and w⁴ is S; or w⁵ is NH, w⁷ is N, w⁶ is C and w⁴ is C; or w⁵ is C, w⁷ is NMe, w⁶ is C and w⁴ is C; or w⁵ is N, w⁷ is C, w⁶ is C and w⁴ is S; or w⁵ is C, w⁷ is C, w⁶ is Sand w⁴ is N.

In more specific embodiments, the present disclosure is directed towards a compound or a pharmaceutically acceptable salt or stereoisomer thereof of formula IV, IVa, IVb, IVc or IVd

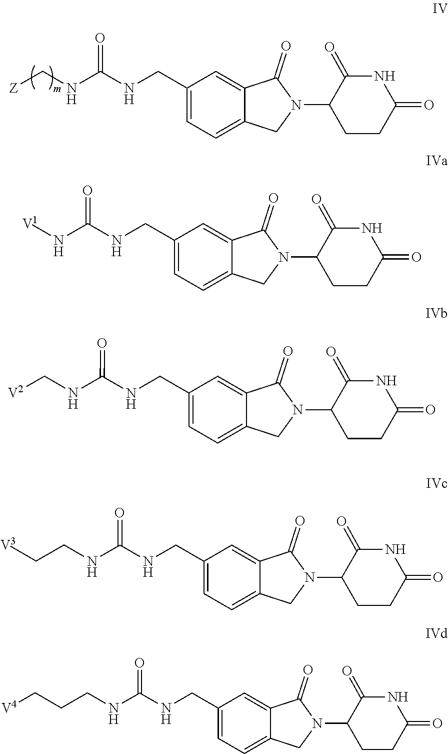

wherein
m is 0, 1, 2 or 3, and
V, V¹, V², V³, V⁴ is selected from

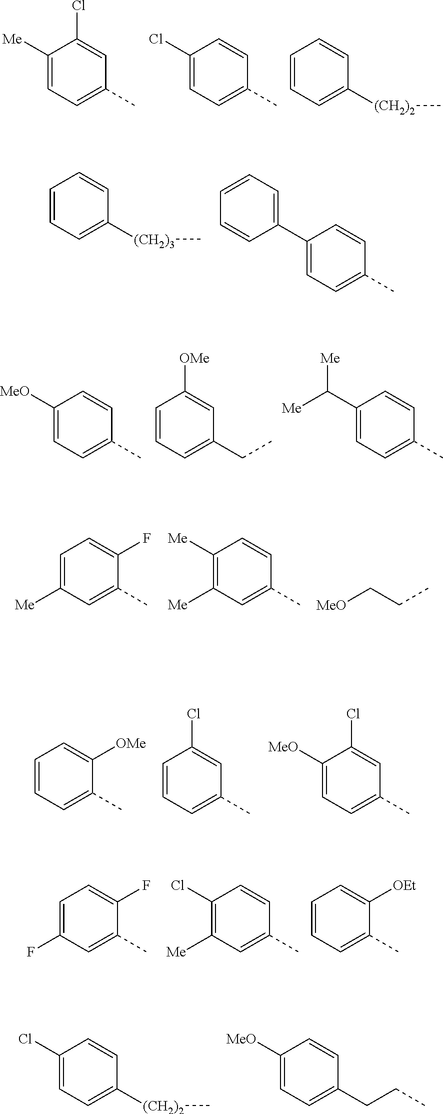

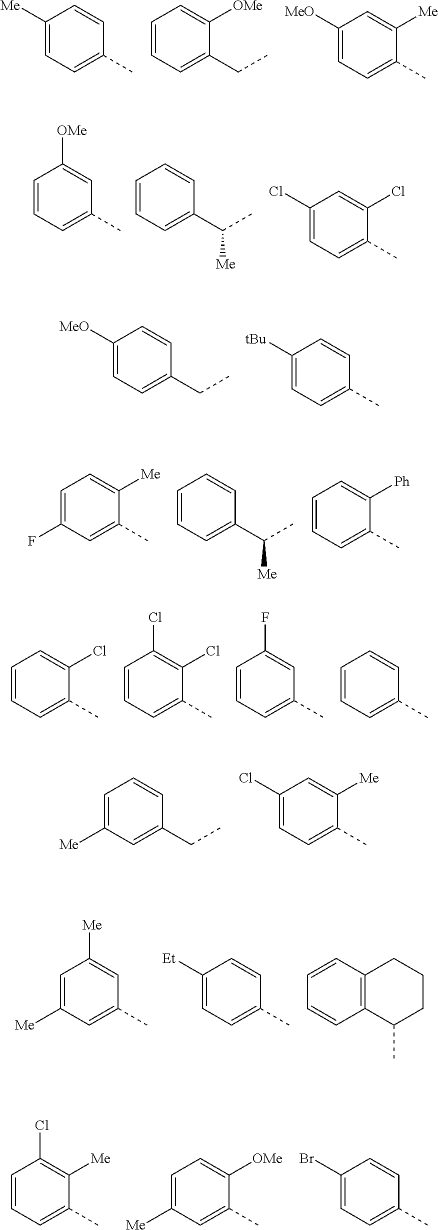

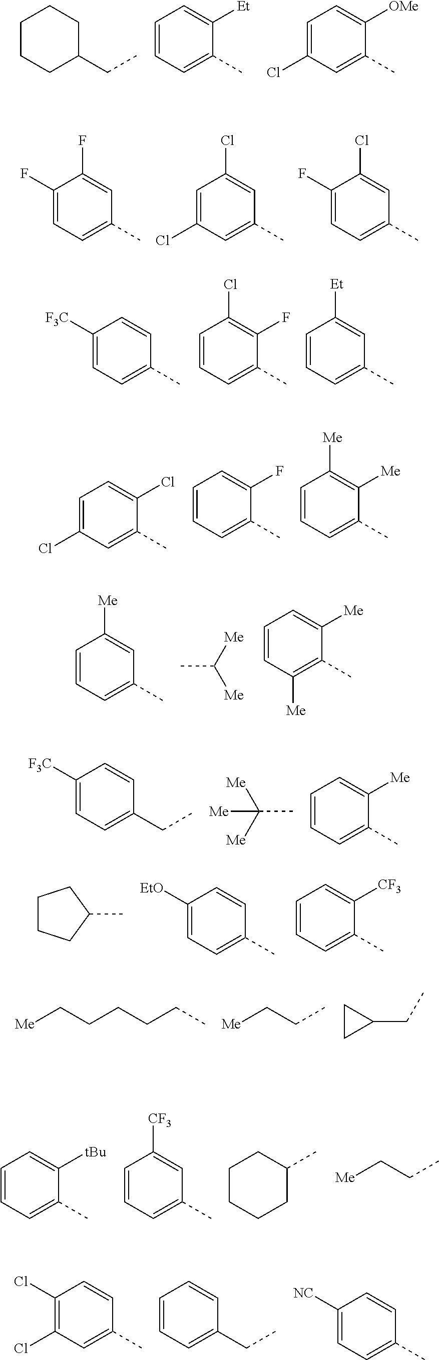

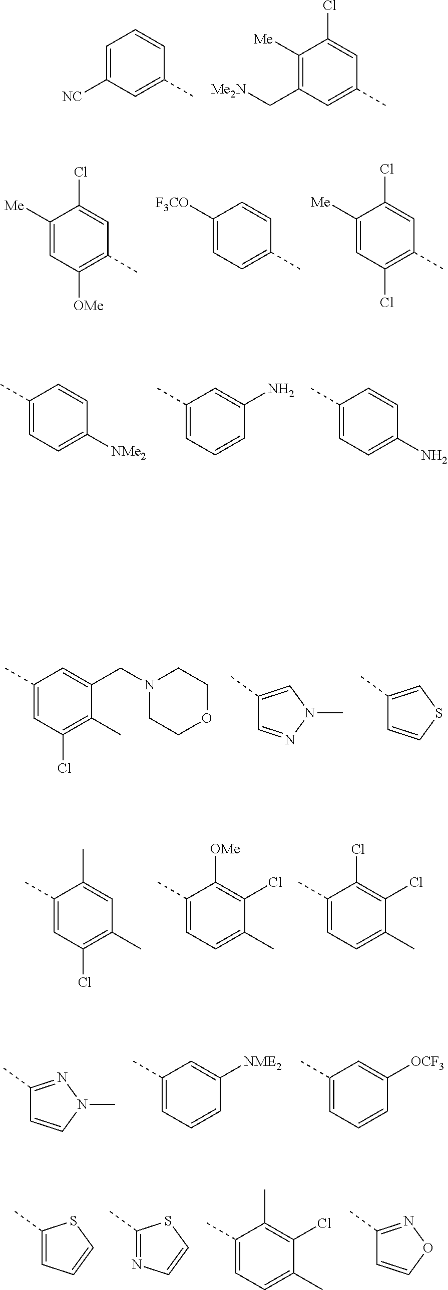

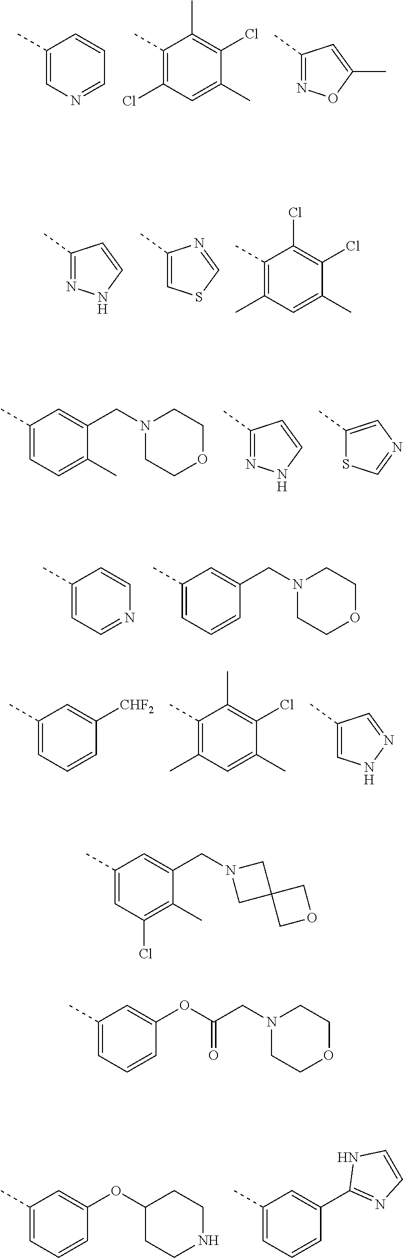

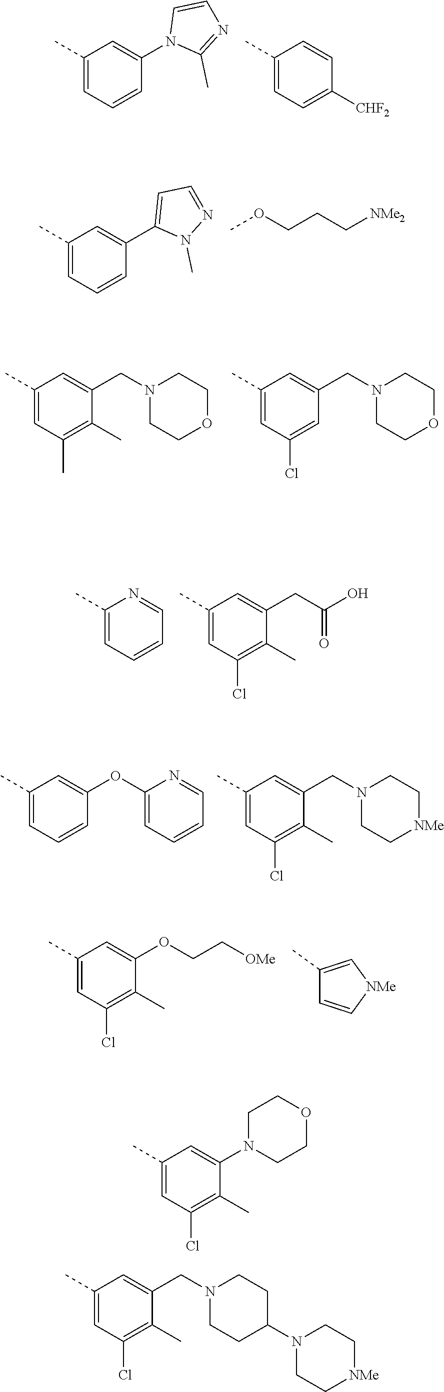

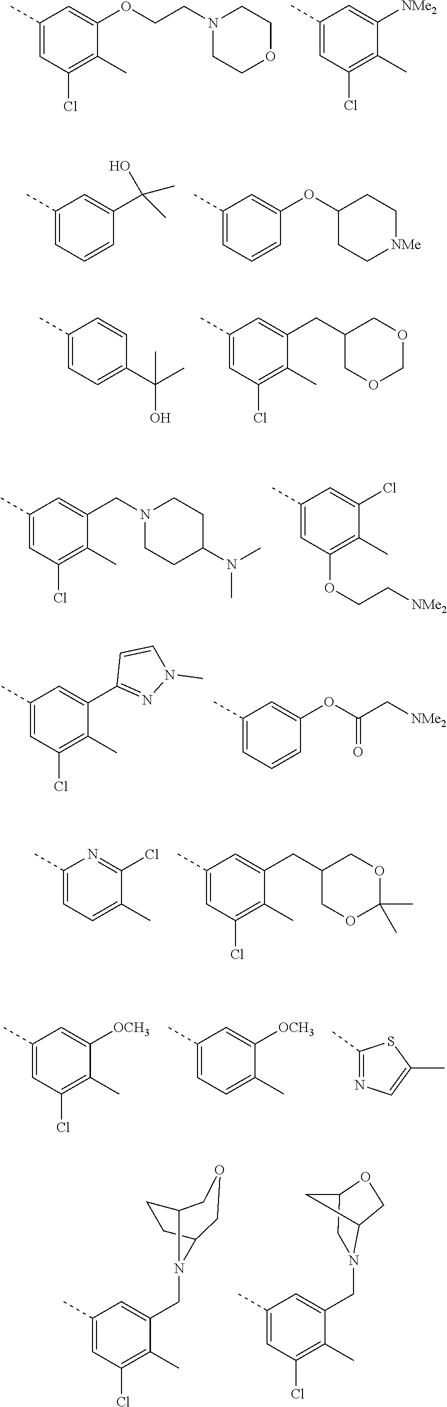

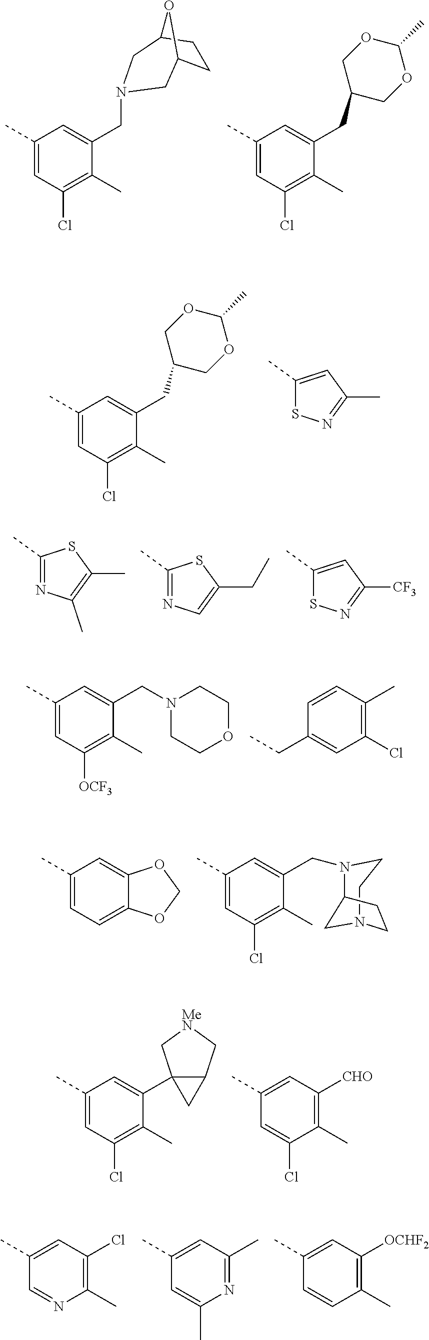

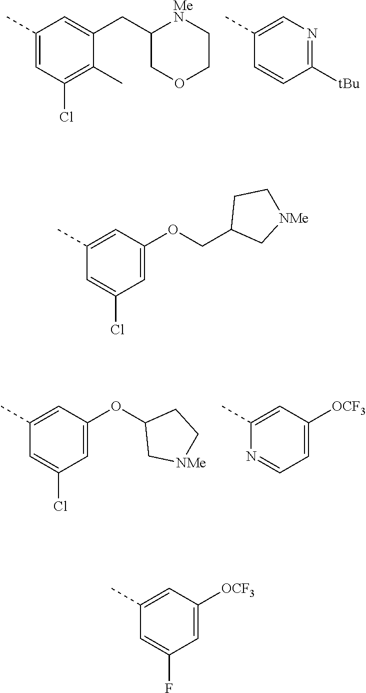

In some embodiments the present disclosure is directed towards a compound or a pharmaceutically acceptable salt or stereoisomer thereof of formula Va:

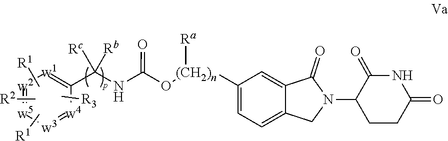

wherein
w¹, w², w³, w⁴, w are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.

In some embodiments of a compound of formula Va, n is 1. In some embodiments n is 1 and R^{a} is H. In some embodiments of a compound of formula Va, n is 1 and R^{a} is methyl. In some embodiments of a compound of formula Va, n is 1, p is 0 and R^{a} is H. In some embodiments of a compound of formula Va, n is 1, p is 0 and R^{a} is methyl

In some embodiments of a compound of formula Va, p is 0. In some embodiments of a compound of formula Va, p is 1. In some embodiments of a compound of formula Va, p is 1, and R^{b} and R^{c} are H. In some embodiments of a compound of formula Va, p is 1, R^{b} is methyl and R^{c} is H.

In some embodiments of formula Va, X⁵ is in the 4-position or in the 5-position or in the 7-position of the ring.

In some embodiments of formula Va, X⁵ is H. In some embodiments of formula Va, X⁵ is C₁₋₄ alkyl, such as methyl, —C₁₋₄ alkoxy, such as —OMe, —CN, halogen, such as F, Cl, Br.

In some embodiments of a compound of formula Va, w¹, w², w³, w⁴, w⁵ are C. In some embodiments of a compound of formula Va, either w¹ or w² or w³ or w⁴ or w⁵ is N and the remaining 4 of w¹, w², w³, w⁴, w⁵ are C. In some embodiments of a compound of formula Va, w¹, w² or w¹, w³ or w¹, w⁴ or w², w³ are N and the remaining 3 of w¹, w², w³, w⁴, w⁵ are C. In some embodiments of a compound of formula Va, w¹, w², w³, w⁴, w⁵ are C.

In some embodiments of a compound of formula Va, L³ is a covalent bond. In some embodiments of a compound of formula Va, L³ is linear or branched C₁₋₄ alkyl, such as —CH₂—. In some embodiments of a compound of formula Va, L³ is —O—. In some embodiments of a compound of formula Va, L³ is linear or branched C₁₋₄ alkoxy, such as —O—CH₂—, —O—(CH₂)₂—.

In some embodiments of a compound of formula Va, R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula Va, R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, R¹ is H, and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula Va, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, n is 1, R^{a} is H and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va, n is 1, R^{a} is H and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, n is 1, R^{a} is H, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va, n is 1, R^{a} is H, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, p is 0 and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va, p is 0 and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, p is 0, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va, p is 1, R^{b}, R^{c} are H, and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is C₃₋₆ cycloalkyl, C₆₋₁₀ aryl, 5-10 membered heteroaryl, 4-8 membered heterocycloalkyl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is —C₁₋₄ alkyl-C₃₋₆ cycloalkyl, —C₁₋₄ alkyl-C₆₋₁₀ aryl, —C₁₋₄ alkyl-(5-10 membered heteroaryl), —C₁₋₄ alkyl-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is —O—C₃₋₆ cycloalkyl, —O—C₆₋₁₀ aryl, —O-(5-10 membered heteroaryl), —O-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is —C₁₋₄ alkoxy-C₃₋₆ cycloalkyl, —C₁₋₄ alkoxy-C₆₋₁₀ aryl, —C₁₋₄ alkoxy-(5-10 membered heteroaryl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl.

In some embodiments of a compound of formula Va, R¹ is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe.

In some embodiments of a compound of formula Va, R¹ is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe.

In some embodiments of a compound of formula Va, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is C₃₋₆ cycloalkyl, C₆₋₁₀ aryl, 5-10 membered heteroaryl, 4-8 membered heterocycloalkyl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is —C₁₋₄ alkyl-C₃₋₆ cycloalkyl, —C₁₋₄ alkyl-C₆₋₁₀ aryl, —C₁₋₄ alkyl-(5-10 membered heteroaryl), —C₁₋₄ alkyl-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is —O—C₃₋₆ cycloalkyl, —O—C₆₋₁₀ aryl, —O-(5-10 membered heteroaryl), —O-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is —C₁₋₄ alkoxy-C₃₋₆ cycloalkyl, —C₁₋₄ alkoxy-C₆₋₁₀ aryl, —C₁₋₄ alkoxy-(5-10 membered heteroaryl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is H; and p is 0.

In some embodiments of a compound of formula Va, R¹ is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula Va, R¹ is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula Va, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is CH₃; and p is 0.

In some embodiments of a compound of formula Va, R¹ is C₆₋₁₀ aryl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n is 1, R^{a} is CH₃; and p is 0.

In some embodiments of a compound of formula Va, R¹ is C₆₋₁₀ aryl, wherein R¹ is unsubstituted or substituted with linear or branched —C₁₋₄ alkyl, e.g. methyl, —C₁₋₄alkoxy, e.g. —OMe, and halogen, such as F, Cl, e.g. Cl; and n is 1, R^{a} is CH₃; and p is 0.

In some embodiments of a compound of formula Va, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n, p are 1, R^{a}, R^{b}, R^{c} are H.

In some embodiments of a compound of formula Va, R¹ is C₆₋₁₀ aryl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n, p are 1, R^{a}, R^{b}, R^{c} are H.

In some embodiments of a compound of formula Va, R¹ is C₆₋₁₀ aryl, wherein R¹ is unsubstituted or substituted with —C₁₋₄ alkoxy, e.g. —OMe; and n, p are 1, R^{a}, R^{b}, R^{c} are H.

In some embodiments of a compound of formula Va, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n, p are 1, R^{a}, R^{c} are H, R^{b} is CH₃.

In some embodiments of a compound of formula Va, R¹ is C₆₋₁₀ aryl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl; and n, p are 1, R^{a}, R^{c} are H, R^{b} is CH₃.

In some embodiments of a compound of formula Va, R¹ is C₆₋₁₀ aryl; and n, p are 1, R^{a}, R^{c} are H, R^{b} is CH₃.

Some embodiments of the compound of formula Va are provided by formula Va-1, wherein w¹ to w⁵ are C, and by formula Va-2, Va-3, and Va-4, wherein one of w¹ to w⁵ is N, more specifically, wherein w¹ is N, w² to w⁵ are C; or w² is N, w¹ and w³ to w⁵ are C; or w³ is N, w¹, w² and w⁴, w⁵ are C

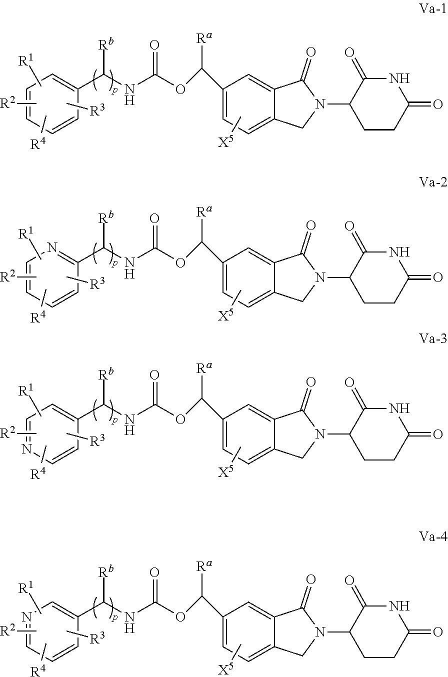

wherein
R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, or —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
X⁵ is H, linear or branched C₁₋₆ alkyl, —C₁₋₄ alkoxy, —CN, halogen, CF₃, CHF₂, CMeF₂, OCF₃, OCHF₂;
R^{a}, R^{b} are independently of each other H or methyl; and p is 0 or 1.

In some embodiments of a compound of formula Va-1, Va-2, Va-3, Va-4, p is 0. In some embodiments of a compound of formula Va-1, Va-2, Va-3, Va-4, p is 1. In some embodiments of a compound of formula Va-1, Va-2, Va-3, Va-4, p is 1 and R^{b} is H. In some embodiments of a compound of formula Va-1, Va-2, Va-3, Va-4, p is 1 and R^{b} is methyl.

In some embodiments of a compound of formula Va-1, Va-2, Va-3, Va-4, p is 0 and R^{a} is H. In some embodiments of a compound of formula Va-1, Va-2, Va-3, Va-4, p is 0 and R^{a} is methyl. In some embodiments of a compound of formula Va-1, Va-2, Va-3, Va-4, p is 1, R^{b} is H and R^{a} is H. In some embodiments of a compound of formula Va-1, Va-2, Va-3, Va-4, p is 1, R^{b} is methyl and R^{a} is H.

In some embodiments of a compound of formula Va-1, Va-2, Va-3, Va-4, L³ is a covalent bond. In some embodiments of a compound of formula Va-1, Va-2, Va-3, Va-4, L³ is linear or branched C₁₋₄ alkyl, such as —CH₂—. In some embodiments of a compound of formula Va-1, Va-2, Va-3, Va-4, L³ is —O—. In some embodiments L³ is linear or branched C₁₋₄ alkoxy, such as —O—CH₂—, —O—(CH₂)₂—.

In some embodiments of formula Va-1, Va-2, Va-3, Va-4, X⁵ is in the 4-position or in the 5-position or in the 7-position of the ring.

In some embodiments of formula Va-1, Va-2, Va-3, Va-4, X⁵ is H. In some embodiments of formula Va-1, Va-2, Va-3, Va-4, X⁵ is C₁₋₄ alkyl, such as methyl, —C₁₋₄ alkoxy, such as —OMe, —CN, halogen, such as F, Cl, Br.

In some embodiments of a compound of formula Va-1, Va-2, Va-3, Va-4, R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va-1, Va-2, Va-3, Va-4, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va-1, Va-2, Va-3, Va-4, R^{a} is H and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R^{a} is H and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or C₁.

In some embodiments of a compound of formula Va-1, Va-2, Va-3, Va-4, R^{a} is H, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R^{a} is H, R¹ is H and R², R³, R⁴ are independently of each other selected from H, linear or branched —C₁₋₆ alkyl, —C₁ 4 alkoxy, CF₃, CHF₂, CMeF₂, OCF₃, OCHF₂, —CN, and halogen, such as F, Cl or Br, e.g. F or Cl.

In some embodiments of a compound of formula Va-1, Va-2, Va-3, Va-4, p is 0 and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, p is 0 and R¹, R², R³, and R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or C₁.

In some embodiments of a compound of formula Va-1, Va-2, Va-3, Va-4, p is 0, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, p is 0, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g. F or Cl.

In some embodiments of a compound of formula Va-1, Va-2, Va-3, Va-4, p is 0, R^{a} is H and R¹, R², R³, and R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, p is 0, R^{a} is H and R¹, R², R³, and R⁴ are independently of each other selected from H, linear or branched —C₁₋₆ alkyl, C₁₋₄ alkoxy, CF₃, CHF₂, CMeF₂, OCF₃, OCHF₂, —CN, and halogen, such as F, Cl or Br, e.g.

F or Cl.

In some embodiments of a compound of formula Va-1, Va-2, Va-3, Va-4, p is 0, R^{a} is H, R¹ is H and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, p is 0, R^{a} is H, R¹ is H and R², R³, R⁴ are independently of each other selected from H, linear or branched —C₁₋₆ alkyl, —C₁₋₄ alkoxy, CF₃, CHF₂, CMeF₂, OCF₃, OCHF₂, —CN, and halogen, such as F, Cl or Br, e.g.

F or Cl.

In some embodiments of a compound of formula, Va-1, Va-2, Va-3, Va-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₃, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R¹ is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl.

In some embodiments of a compound of formula Va-1, Va-2, Va-3, Va-4, R¹ is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₃, CMeF₂, —OCF₃, OCHF₂, CN, and C₁₋₄ alkoxy, e.g. —OMe.

In some embodiments of a compound of formula Va-1, Va-2, Va-3, Va-4, R¹ is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe.

In some embodiments of a compound of formula Va-1, Va-2, Va-3, Va-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl; and n is 1, R^{a} is H; and p is 0.

In some embodiments of a compound of formula Va-1, Va-2, Va-3, Va-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆ alkylC(O)O—C₁₋₆alkyl, NH₂, —C₁₋₄ alkylhydroxy, and halogen, such as F, Cl or Br, e.g. F or Cl; and n is 1, R^{a} is H; and p is 0.

In some embodiments of a compound of formula Va-1, Va-2, Va-3, Va-4, R¹ is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₄alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O-C₆alkyl, —COOH, —C₁₋₆alkylC(O)OH, —C₁₋₆alkylC(O)O—C₁₋₆alkyl, NH₂, —C₁₋₄ alkylhydroxy, and halogen, such as F, Cl or Br, e.g. F or Cl; and n is 1, R^{a} is H; and p is 0.

In some embodiments of a compound of formula Va-1, Va-2, Va-3, Va-4, R¹ is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula Va-1, Va-2, Va-3, Va-4, R¹ is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

Some embodiments of the compound of formula Va are also provided by formula Va-5, Va-6, Va-7, Va-8, Va-9, and Va-10, wherein two of w¹ to w⁵ are N, for example, wherein w¹, w² are N, w³ to w⁵ are C; or w¹, w⁵ are N, w² to w⁴ are C; or w², w⁴ are N, w¹, w³, w⁵ are C; or w¹, w³ are N, w², w⁴, w⁵ are C; or w², w³ are N, w², w³, w⁴, w⁵ are C; or w¹, w⁴ are N, w², w³, w⁵ are C

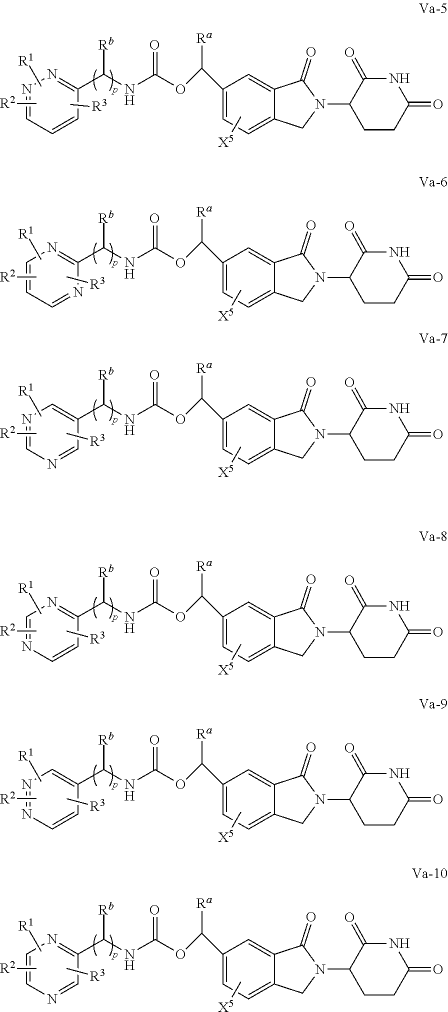

wherein
R¹, R², R³, R⁴ are independently of each other selected from H, linear or branched —C₁₋₆ alkyl, linear or branched C₁₋₆ heteroalkyl, —C₁₋₄ alkoxy, CF₃, CHF₂, CMeF₂, —O—(CH₂)₂—OMe, OCF₃, OCHF₂, —C₁₋₆ alkylamino, —CN, NH₂, —C₁₋₄ alkylhydroxy, and halogen, such as F, Cl or Br, e.g. F or Cl, or a group of formula -L³-X², wherein L³ is a covalent bond, linear or branched C₁₋₆ alkyl, —O—, or —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
X⁵ is H, linear or branched C₁₋₆ alkyl, —C₁₋₄ alkoxy, —CN, halogen, CF₃, CHF₂, CMeF₂, OCF₃, OCHF₂;
R^{a}, R^{b} are independently of each other H or methyl; and p is 0 or 1.

In some embodiments of a compound of formula Va-5, Va-6, Va-7, Va-8, Va-9, Va-10, p is 0. In some embodiments of a compound of formula Va-5, Va-6, Va-7, Va-8, Va-9, Va-10, p is 1.

In some embodiments of a compound of formula Va-5, Va-6, Va-7, Va-8, Va-9, Va-10, R^{a} is H. In some embodiments of a compound of formula Va-5, Va-6, Va-7, Va-8, Va-9, Va-10, R^{a} is methyl. In some embodiments of a compound of formula Va-5, Va-6, Va-7, Va-8, Va-9, Va-10, p is 0 and R^{a} is H. In some embodiments of a compound of formula Va-5, Va-6, Va-7, Va-8, Va-9, Va-10, p is 0 and R^{a} is methyl.

In some embodiments of a compound of formula Va-5, Va-6, Va-7, Va-8, Va-9, Va-10, p is 1 and R^{b} is H. In some embodiments of a compound of formula Va-5, Va-6, Va-7, Va-8, Va-9, Va-10, p is 1 and R^{b} is methyl. In some embodiments of a compound of formula Va-5, Va-6, Va-7, Va-8, Va-9, Va-10, p is 1, R^{b} is H and R^{a} is H. In some embodiments of a compound of formula Va-5, Va-6, Va-7, Va-8, Va-9, Va-10, p is 1, R^{b} is methyl and R^{a} is H.

In some embodiments of formula Va-5, Va-6, Va-7, Va-8, Va-9, and Va-10, X⁵ is in the 4-position or in the 5-position or in the 7-position of the ring.

In some embodiments of formula Va-5, Va-6, Va-7, Va-8, Va-9, and Va-10, X⁵ is H. In some embodiments of formula Va-5, Va-6, Va-7, Va-8, Va-9, and Va-10, X⁵ is C₁₋₄ alkyl, such as methyl, —C₁₋₄ alkoxy, such as —OMe, —CN, halogen, such as F, Cl, Br.

In some embodiments of a compound of formula Va-5, Va-6, Va-7, Va-8, Va-9, and Va-10, R¹, R², R³ each are independently selected from hydrogen, linear or branched C₁₋₄ alkyl, C₁₋₄ alkoxy, pyridinyl, pyrrolidinyl, N-methyl pyrrolidinyl, piperdinyl, N-methyl piperdinyl, morpholinyl, oxetanyl, methyl oxetanyl, furanyl, piperazinyl, N-methyl piperazinyl, azetidinyl, methyl azetidinyl, —C₁₋₄ alkyl-pyrrolidinyl, —C₁₋₄ alkyl-morpholinyl, —C₁₋₄ alkyl-(N-methyl-pyrrolidinyl), —C₁₋₄ alkoxyl-pyrrolidinyl, —C₁₋₄ alkoxyl-morpholinyl, —C₁₋₄ alkoxyl-(N-methyl-pyrrolidinyl), —O-pyrrolidinyl, —O-morpholinyl, —O—(N-methyl-pyrrolidinyl), 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, phenyl, CF₃, CHF₂, CMeF₂, —O—(CH₂)₂—OMe, OCF₃, OCHF₂, C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, C₆ aryloxy and halogen, such as F, Cl or Br, e.g. F or Cl.

In specific embodiments of a compound of formula Va-5, Va-6, Va-7, Va-8, Va-9, and Va-10, R¹, R², R³ each are independently selected from hydrogen, linear or branched C₁₋₄ alkyl, C₁₋₄ alkoxy, pyridinyl, pyrrolidinyl, N-methyl pyrrolidinyl, piperdinyl, morpholinyl, oxetanyl, methyl oxetanyl, furanyl, —C₁₋₄ alkyl-pyrrolidinyl, —C₁₋₄ alkyl-morpholinyl, —C₁₋₄ alkyl-(N-methyl-pyrrolidinyl), —C₁₋₄ alkoxyl-pyrrolidinyl, —C₁₋₄ alkoxyl-morpholinyl, —C₁₋₄ alkoxyl-(N-methyl-pyrrolidinyl), —O-pyrrolidinyl, —O-morpholinyl, —O—(N-methyl-pyrrolidinyl), 1,4-diazabicyclo[3.2.1]octan-4-yl, 3-methyl-3-azabicyclo[3.1.0]hexan-1-yl, 8-oxa-3-azabicyclo[3.2.1]octan-3-yl, phenyl, CF₃, CHF₂, CMeF₂, OCF₃, OCHF₂, C₁₋₄ alkylhydroxy, C₆ aryloxy and halogen, such as F, Cl or Br, e.g. F or Cl.

In some embodiments of a compound of formula Va-5, Va-6, Va-7, Va-8, Va-9, Va-10, R¹ is 4-8 membered heterocycloalkyl, which is unsubstituted or substituted with one or more of linear or branched C₁₋₆ alkyl, halogen, CF₃, CHF₂, CMeF₂, OCF₃, OCHF₂; and R², R³ each are independently selected from H, linear or branched C₁₋₄ alkyl, CF₃, CHF₂, CMeF₂, OCF₃, OCHF₂, and halogen, such as F, Cl or Br, e.g. F or Cl.

In some embodiments of a compound of formula Va-5, Va-6, Va-7, Va-8, Va-9, Va-10, R¹ is selected from pyrrolidinyl, piperidinyl, piperazinyl, morpholinyl; and R², R³ each are independently selected from H, linear or branched C₁₋₄ alkyl, CF₃, CHF₂, CMeF₂, OCF₃, OCHF₂, and halogen, such as F, Cl or Br, e.g. F or Cl.

In some embodiments of a compound of formula Va-5, Va-6, Va-7, Va-8, Va-9, Va-10, R¹ is piperidinyl; and R², R³ each are independently selected from H, linear or branched C₁₋₄ alkyl, CF₃, CHF₂, CMeF₂, OCF₃, OCHF₂, and halogen, such as F, Cl or Br, e.g. F or Cl.

In some embodiments, the present disclosure is directed towards a compound containing a fused 6 (saturated)-6 (aromatic) ring system or a fused 5 (saturated)-6 (aromatic) ring system of formula Vb:

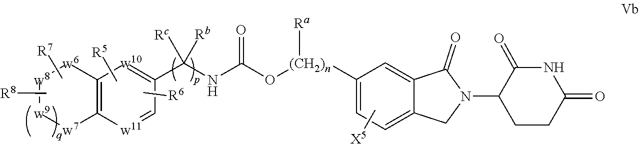

wherein one or two of w⁶, w⁷, w⁸, w⁹ are selected from C and O and the remaining of w⁶, w⁷, w⁸, w⁹ are C; w¹⁰, w¹¹ are independently of each other selected from C and N;
X⁵ is H, linear or branched C₁₋₆ alkyl, —C₁₋₄ alkoxy, —CN, halogen, CF₃, CHF₂, CMeF₂, OCF₃, OCHF₂;
R⁵, R⁶, R⁷ R⁸ are independently of each other selected from H, linear or branched C₁₋₄ alkyl, halogen, such as F or Cl, e.g. F;
R^{a} is H, linear or branched C₁₋₄ alkyl; R^{b}, R^{c} are independently of each other H, linear or branched C₁₋₄ alkyl; q is 0, 1; n is 1 or 2; and p is 0 or 1.

In some embodiments of a compound of formula Vb, R^{a} is H. In some embodiments of a compound of formula Vb, R^{a} is methyl. In some embodiments of a compound of formula Vb, n is 1. In some embodiments of a compound of formula Vb, n is 1 and R^{a} is H. In some embodiments of a compound of formula Vb, n is 1 and R^{a} is methyl.

In some embodiments of a compound of formula Vb, p is 0. In some embodiments of a compound of formula Vb, p is 0 and R^{a} is H. In some embodiments of a compound of formula Vb, p is 0 and R^{a} is methyl. In some embodiments of a compound of formula Vb, p is 1. In some embodiments of a compound of formula Vb, p is 1, and R^{b} and R^{c} are H. In some embodiments of a compound of formula Vb, p is 1, R^{b} is methyl and R^{c} is H.

In some embodiments of a compound of formula Vb, one of w¹⁰ and w¹¹ is C. In some embodiments of a compound of formula Vb, one of w¹⁰ and w¹¹ is C and the other is N.

In some embodiments of a compound of formula Vb, q is 0 and w⁸ is C. In some embodiments of a compound of formula Vb, q is 0, w⁸ is C and w⁶, w⁷ are selected from C and O. In some embodiments of a compound of formula Vb, q is 0, w⁸ is C and w⁶, w⁷ are O. In some embodiments of a compound of formula Vb, q is 0, w⁸ is C and one of w⁶, w⁷ is C and the other of w⁶, w⁷ is O.

In some embodiments of a compound of formula Vb, q is 1, and w⁶, w⁷, w⁸, w⁹ are C. In some embodiments of a compound of formula Vb, q is 1, and w⁶ is O and w⁷, w⁸, w⁹ are C. In some embodiments of a compound of formula Vb, q is 1, and w⁷ is O and w⁶, w⁸, w⁹ are C. In some embodiments of a compound of formula Vb, q is 1, and w⁸ is O and w⁶, w⁷, w⁹ are C. In some embodiments of a compound of formula Vb, q is 1, and w⁹ is 0 and w⁶, w⁷, w⁸ are C.

In some embodiments of formula Vb, X⁵ is in the 4-position or in the 5-position or in the 7-position of the ring.

In some embodiments of formula Vb, X⁵ is H. In some embodiments of formula Vb, X⁵ is C₁₋₄ alkyl, such as methyl, —C₁₋₄ alkoxy, such as —OMe, —CN, halogen, such as F, Cl, Br.

In some embodiments of a compound of formula Vb, R⁵, R⁶ are H.

In some embodiments of a compound of formula Vb, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula Vb are provided by formula Vb′

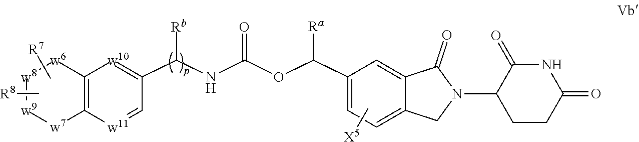

wherein one or two of w⁶, w⁷, w⁸, w⁹ are selected from C and O and the remaining of w⁶, w⁷, w⁸, w⁹ are C; w¹⁰, w¹¹ are independently of each other selected from C and N;
X⁵ is H, linear or branched C₁₋₆ alkyl, —C₁₋₄ alkoxy, —CN, halogen, CF₃, CHF₂, CMeF₂, OCF₃, OCHF₂;
R⁷, R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl; and p is 0 or 1.

In some embodiments of a compound of formula Vb′, R^{a} is H. In some embodiments of a compound of formula Vb′, R^{a} is methyl. In some embodiments of a compound of formula Vb′, p is 0 and R^{a} is H. In some embodiments of a compound of formula Vb′, p is 0 and R^{a} is methyl. In some embodiments of a compound of formula Vb′, p is 1 and R^{b} is H. In some embodiments of a compound of formula Vb′, p is 1 and R^{b} is methyl.

In some embodiments of a compound of formula Vb′, one of w¹⁰ and w¹¹ are C. In some embodiments of a compound of formula Vb′, one of w¹⁰ an d w¹¹ is C and the other is N.

In some embodiments of a compound of formula Vb′, w⁶, w⁷, w⁸, w⁹ are C. In some embodiments of a compound of formula Vb′, w⁶ is O and w⁷, w⁸, w⁹ are C. In some embodiments of a compound of formula Vb′, w⁷ is O and w⁶, w⁸, w⁹ are C. In some embodiments of a compound of formula Vb′, w⁸ is O and w⁶, w⁷, w⁹ are C. In some embodiments of a compound of formula Vb′, w⁹ is O and w⁶, w⁷, w⁸ are C.

In some embodiments of formula Vb′, X⁵ is in the 4-position or in the 5-position or in the 7-position of the ring.

In some embodiments of formula Vb′, X⁵ is H. In some embodiments of formula Vb′, X⁵ is C₁ 4 alkyl, such as methyl, —C₁₋₄ alkoxy, such as —OMe, —CN, halogen, such as F, Cl, Br.

In some embodiments of a compound of formula Vb′, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula Vb and Vb′ are provided by formula Vb-1, Vb-2, Vb-3

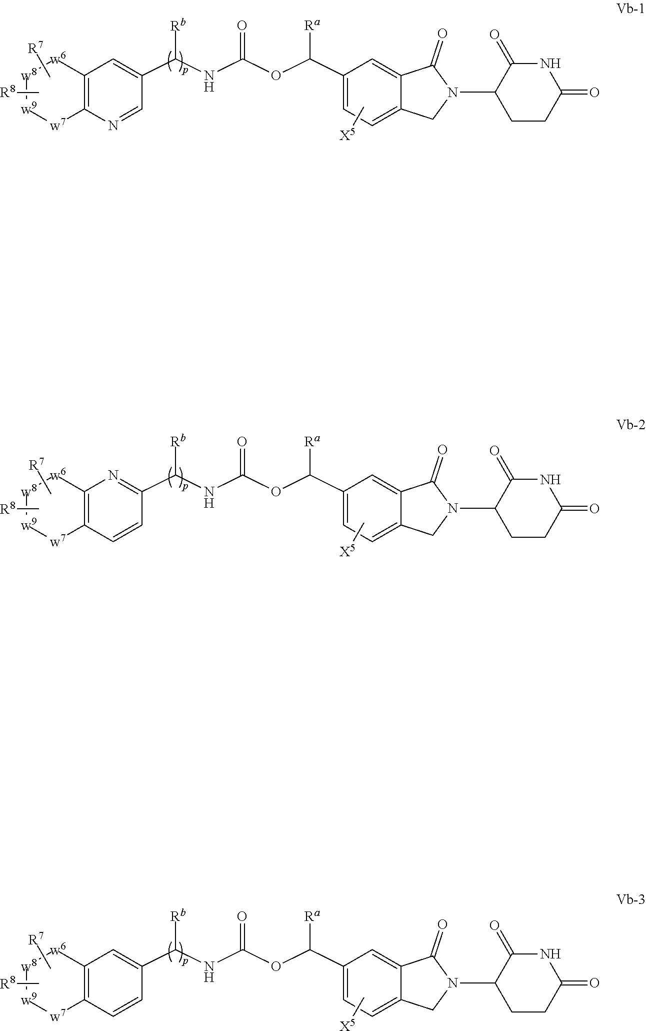

wherein one or two of w⁶, w⁷, w⁸, w⁹ are selected from C and O and the remaining of w⁶, w⁷, w⁸, w⁹ are C;
X⁵ is H, linear or branched C₁₋₆ alkyl, —C₁₋₄ alkoxy, —CN, halogen, CF₃, CHF₂, CMeF₂, OCF₃, OCHF₂;
R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl; and p is 0 or 1.

In some embodiments of a compound of formula Vb-1, Vb-2 or Vb-3, R^{a} is H. In some embodiments of a compound of formula Vb-1, Vb-2 or Vb-3, R^{a} is methyl. In some embodiments of a compound of formula Vb-1, Vb-2 or Vb-3, p is 0 and R^{a} is H. In some embodiments of a compound of formula Vb-1, Vb-2 or Vb-3, p is 0 and R^{a} is methyl.

In some embodiments of a compound of formula Vb-1, Vb-2 or Vb-3, p is 1 and R^{b} is H. In some embodiments of a compound of formula Vb-1, Vb-2 or Vb-3, p is 1 and R^{b} is methyl.

In some embodiments of a compound of formula Vb-1, Vb-2 or Vb-3, p is 1, R^{b} is H and R^{a} is H. In some embodiments of a compound of formula Vb-1, Vb-2 or Vb-3, p is 1, R^{b} is methyl and R^{a} is H.

In some embodiments of a compound of formula Vb-1, Vb-2 or Vb-3, w⁶, w⁷, w⁸, w⁹ are C.

In some embodiments of a compound of formula Vb-1, Vb-2 or Vb-3, w⁶ is O and w⁷, w⁸, w⁹ are C. In some embodiments of a compound of formula Vb-1, Vb-2 or Vb-3, w⁷ is O and w⁶, w⁸, w⁹ are C. In some embodiments of a compound of formula Vb-1, Vb-2 or Vb-3, w⁸ is O and w⁶, w⁷, w⁹ are C. In some embodiments of a compound of formula Vb-1, Vb-2 or Vb-3, w⁹ is O and w⁶, w⁷, w⁸ are C.

In some embodiments of formula Vb-1, Vb-2 or Vb-3, X⁵ is in the 4-position or in the 5-position or in the 7-position of the ring.

In some embodiments of formula Vb-1, Vb-2 or Vb-3, X⁵ is H. In some embodiments of formula Vb-1, Vb-2 or Vb-3, X⁵ is C₁₋₄ alkyl, such as methyl, —C₁₋₄ alkoxy, such as —OMe, —CN, halogen, such as F, Cl, Br.

In some embodiments of a compound of formula Vb-1, Vb-2 or Vb-3, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula Vb-1 are provided by formula Vb-1a, Vb-1b, Vb-1c, and Vb-1d

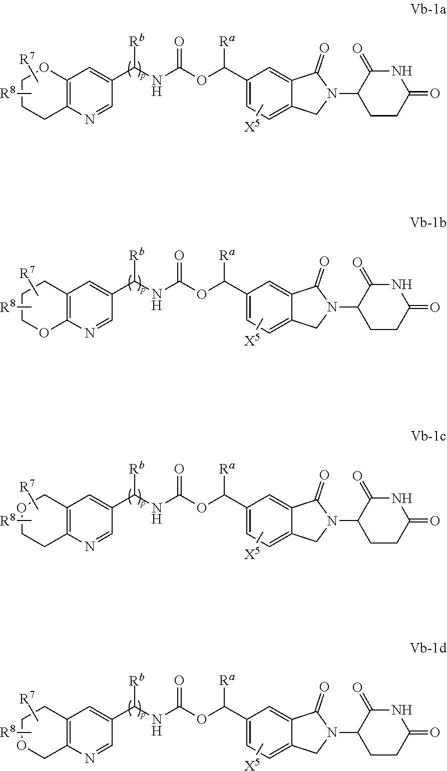

wherein
X⁵ is H, C₁₋₄ alkyl, such as methyl, —C₁₋₄ alkoxy, such as —OMe, —CN, halogen, such as F, Cl, Br;
R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl, and p is 0 or 1.

In some embodiments of a compound of formula Vb-1a, Vb-1b, Vb-1c, and Vb-1d, p is 0.

In some embodiments of a compound of formula Vb-1a, Vb-1b, Vb-1c, and Vb-1d, p is 1.

In some embodiments of a compound of formula Vb-1a, Vb-1b, Vb-1c, and Vb-1d, R^{a} is H.

In some embodiments of a compound of formula Vb-1a, Vb-1b, Vb-1c, and Vb-1d, R^{a} is methyl. In some embodiments of a compound of formula Vb-1a, Vb-1b, Vb-1c, and Vb-1d, p is 0 and R^{a} is H. In some embodiments of a compound of formula Vb-1a, Vb-1b, Vb-1c, and Vb-1d, p is 0 and R^{a} is methyl.

In some embodiments of a compound of formula Vb-1a, Vb-1b, Vb-1c, and Vb-1d, p is 1 and R^{b} is H. In some embodiments of a compound of formula Vb-1a, Vb-1b, Vb-1c, and Vb-1d, p is 1 and R^{b} is methyl. In some embodiments of a compound of formula Vb-1a, Vb-1b, Vb-1c, and Vb-1d, p is 1, R^{b} is H and R^{a} is H. In some embodiments of a compound of formula Vb-1a, Vb-1b, Vb-1c, and Vb-1d, p is 1, R^{b} is methyl and R^{a} is H.

In some embodiments of formula Vb-1a, Vb-1b, Vb-1c, and Vb-1d, X⁵ is in the 4-position or in the 5-position or in the 7-position of the ring.

In some embodiments of formula Vb-1a, Vb-1b, Vb-1c, and Vb-1d, X⁵ is H. In some embodiments of formula Vb-1a, Vb-1b, Vb-1c, and Vb-1d, X⁵ is methyl, —OMe, —CN, F, Cl, Br.

In some embodiments of a compound of formula Vb-1a, Vb-1b, Vb-1c, and Vb-1d, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula Vb-2 are provided by formula Vb-2a, Vb-2b, Vb-2c, or Vb-2d

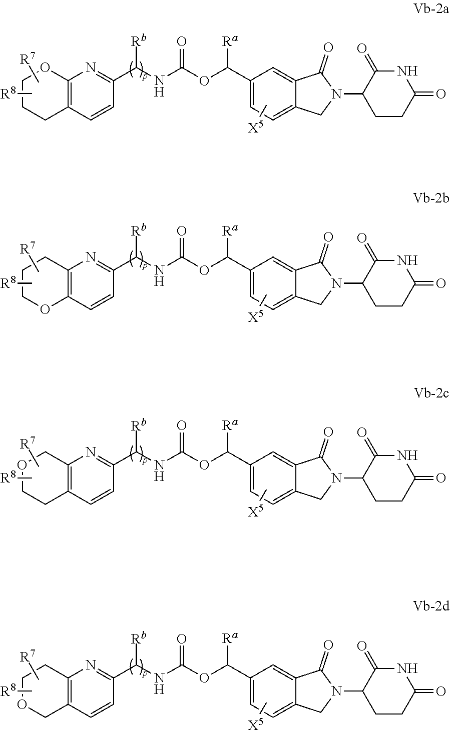

wherein
X⁵ is H, C₁₋₄ alkyl, such as methyl, —C₁₋₄ alkoxy, such as —OMe, —CN, halogen, such as F, Cl, Br; R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl, and p is 0 or 1.

In some embodiments of a compound of formula Vb-2a, Vb-2b, Vb-2c, or Vb-2d, p is 0. In some embodiments of a compound of formula Vb-2a, Vb-2b, Vb-2c, or Vb-2d, p is 1.

In some embodiments of a compound of formula Vb-2a, Vb-2b, Vb-2c, or Vb-2d, R^{a} is H. In some embodiments of a compound of formula Vb-2a, Vb-2b, Vb-2c, or Vb-2d, R^{a} is methyl. In some embodiments of a compound of formula Vb-2a, Vb-2b, Vb-2c, or Vb-2d, p is 0 and R^{a} is H. In some embodiments of a compound of formula Vb-2a, Vb-2b, Vb-2c, or Vb-2d, p is 0 and R^{a} is methyl.

In some embodiments of a compound of formula Vb-2a, Vb-2b, Vb-2c, or Vb-2d, p is 1 and R^{b} is H. In some embodiments of a compound of formula Vb-2a, Vb-2b, Vb-2c, or Vb-2d, p is 1 and R^{b} is methyl. In some embodiments of a compound of formula Vb-2a, Vb-2b, Vb-2c, or Vb-2d, p is 1, R^{b} is H and R^{a} is H. In some embodiments of a compound of formula Vb-2a, Vb-2b, Vb-2c, or Vb-2d, p is 1, R^{b} is methyl and R^{a} is H.

In some embodiments of formula Vb-2a, Vb-2b, Vb-2c, or Vb-2d, X⁵ is in the 4-position or in the 5-position or in the 7-position of the ring.

In some embodiments of formula Vb-2a, Vb-2b, Vb-2c, or Vb-2d, X⁵ is H. In some embodiments of formula Vb-2a, Vb-2b, Vb-2c, or Vb-2d, X⁵ is methyl, —OMe, —CN, F, Cl, Br.

Some embodiments of a compound of formula Vb-3 are provided by formula Vb-3a, Vb-3b, Vb-3c, or Vb-3d

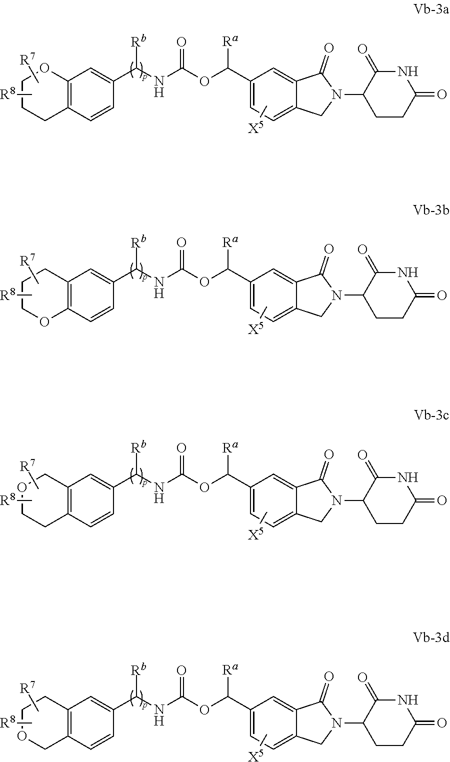

wherein
X⁵ is H, C₁₋₄ alkyl, such as methyl, —C₁₋₄ alkoxy, such as —OMe, —CN, halogen, such as F, Cl, Br;
R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl, and p is 0 or 1.

In some embodiments of a compound of formula Vb-3a, Vb-3b, Vb-3c, or Vb-3d, p is 0. In some embodiments of a compound of formula Vb-3a, Vb-3b, Vb-3c, or Vb-3d, p is 1.

In some embodiments of a compound of formula Vb-3a, Vb-3b, Vb-3c, or Vb-3d, R^{a} is H. In some embodiments of a compound of formula Vb-3a, Vb-3b, Vb-3c, or Vb-3d, R^{a} is methyl. In some embodiments of a compound of formula Vb-3a, Vb-3b, Vb-3c, or Vb-3d, p is 0 and R^{a} is H. In some embodiments of a compound of formula Vb-3a, Vb-3b, Vb-3c, or Vb-3d, p is 0 and R^{a} is methyl.

In some embodiments of a compound of formula Vb-3a, Vb-3b, Vb-3c, or Vb-3d, p is 1 and R^{b} is H. In some embodiments of a compound of formula Vb-3a, Vb-3b, Vb-3c, or Vb-3d, p is 1 and R^{b} is methyl. In some embodiments of a compound of formula Vb-3a, Vb-3b, Vb-3c, or Vb-3d, p is 1, R^{b} is H and R^{a} is H. In some embodiments of a compound of formula Vb-3a, Vb-3b, Vb-3c, or Vb-3d, p is 1, R^{b} is methyl and R^{a} is H.

In some embodiments of formula Vb-3a, Vb-3b, Vb-3c, or Vb-3d, X⁵ is in the 4-position or in the 5-position or in the 7-position of the ring.

In some embodiments of formula Vb-3a, Vb-3b, Vb-3c, or Vb-3d, X⁵ is H. In some embodiments of formula Vb-3a, Vb-3b, Vb-3c, or Vb-3d, X⁵ is methyl, —OMe, —CN, F, Cl, Br.

In some embodiments, the compound of formula Va q is 0 and is provided by a compound of formula Vb-5

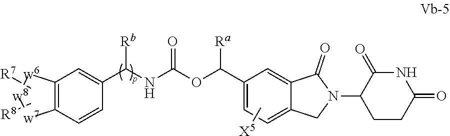

wherein
w⁶, w⁷, w⁸ are independently of each other selected from C and O;
X⁵ is H, linear or branched C₁₋₆ alkyl, —C₁₋₄ alkoxy, —CN, halogen, CF₃, CHF₂, CMeF₂, OCF₃, OCHF₂;
R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl and p is 0 or 1.

In some embodiments of a compound of formula Vb-5, w⁶, w⁷, w⁸ are independently of each other selected from C and O; with the proviso that neighbouring groups cannot be both O.

In some embodiments of a compound of formula Vb-5, p is 0. In some embodiments of a compound of formula Vb-5, p is 1.

In some embodiments of a compound of formula Vb-5, R^{a} is H. In some embodiments of a compound of formula Vb-5, R^{a} is methyl. In some embodiments of a compound of formula Vb-5, p is 0 and R^{a} is H. In some embodiments of a compound of formula Vb-5, p is 0 and R^{a} is methyl.

In some embodiments of a compound of formula Vb-5, p is 1 and R^{b} is H. In some embodiments of a compound of formula Vb-5, p is 1 and R^{b} is methyl. In some embodiments of a compound of formula Vb-5, p is 1, R^{b} is H and R^{a} is H. In some embodiments of a compound of formula Vb-5, p is 1, R^{b} is methyl and R^{a} is H.

In some embodiments of a compound of formula Vb-5, w⁵ is C. In some embodiments of a compound of formula Vb-5, w⁵ is C and w⁶, w⁷ are selected from C and O. In some embodiments of a compound of formula Vb-5, w⁵ is C and w⁶, w⁷ are O. In some embodiments of a compound of formula Vb-5, w⁵ is C and one of w⁶, w⁷ is C and the other of w⁶, w⁷ is O.

In some embodiments of formula Vb-5, X⁵ is in the 4-position or in the 5-position or in the 7-position of the ring.

In some embodiments of formula Vb-5, X⁵ is H. In some embodiments of formula Vb-5, X⁵ is C₁₋₄ alkyl, such as methyl, —C₁₋₄ alkoxy, such as —OMe, —CN, halogen, such as F, Cl, Br.

In some embodiments of a compound of formula Vb-5, R⁷ R⁸ are independently of each other selected from H, linear or branched C₁₋₄ alkyl, halogen, such as F or Cl, e.g. F. R⁷ R⁸ may be attached to the same ring atom or to different ring atoms.

In some embodiments the compound of formula Vb and Vb-5 is provided by formula Vb-5a, Vb-5b, Vb-5c, and Vb-5d

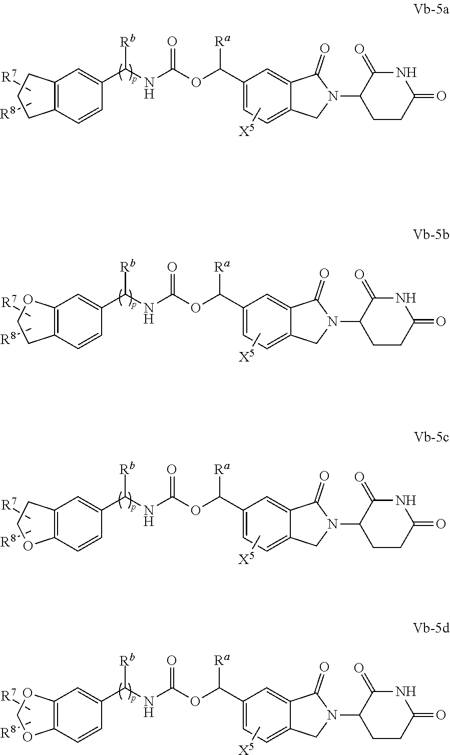

wherein
X⁵ is H, C₁₋₄ alkyl, such as methyl, —C₁₋₄ alkoxy, such as —OMe, —CN, halogen, such as F, Cl, Br;
R⁷ R⁸ are independently of each other selected from H, linear or branched C₁₋₄ alkyl, such as methyl, halogen, such as F or Cl, e.g. F; R^{a}, R^{b} are independently of each other H, linear or branched C₁₋₄ alkyl, and p is 0 or 1.

In some embodiments of formula Vb-5a, Vb-5b, Vb-5c, and Vb-5d, X⁵ is H. In some embodiments of formula Vb-5a, Vb-5b, Vb-5c, and Vb-5d, X⁵ is methyl, —OMe, —CN, F, Cl, Br.

In some embodiments of a compound of formula Vb-5a, Vb-5b, Vb-5c, and Vb-5d, p is 0. In some embodiments of a compound of formula Vb-5a, Vb-5b, Vb-5c, and Vb-5d, p is 1.

In some embodiments of a compound of formula Vb-5a, Vb-5b, Vb-5c, and Vb-5d, R^{a} is H.

In some embodiments of a compound of formula Vb-5a, Vb-5b, Vb-5c, and Vb-5d, R^{a} is methyl. In some embodiments of a compound of formula Vb-5a, Vb-5b, Vb-5c, and Vb-5d, p is 0 and R^{a} is H. In some embodiments of a compound of formula Vb-5a, Vb-5b, Vb-5c, and Vb-5d, p is 0 and R^{a} is methyl.

In some embodiments of a compound of formula Vb-5a, Vb-5b, Vb-5c, and Vb-5d, p is 1 and R^{b} is H. In some embodiments of a compound of formula Vb-5a, Vb-5b, Vb-5c, and Vb-5d, p is 1 and R^{b} is methyl. In some embodiments of a compound of formula Vb-5a, Vb-5b, Vb-5c, and Vb-5d, p is 1, R^{b} is H and R^{a} is H. In some embodiments of a compound of formula Vb-5a, Vb-5b, Vb-5c, and Vb-5d, p is 1, R^{b} is methyl and R^{a} is H.

In some embodiments of a compound of formula Vb-5a, Vb-5b, Vb-5c, and Vb-5d, R¹, R⁸ are independently of each other selected from H, linear or branched C₁₋₄ alkyl, such as methyl, halogen, such as F or Cl, e.g. F. In some embodiments of a compound of formula Vb-5a, Vb-5b, Vb-5c, and Vb-5d, R⁷, R⁸ are H. In some embodiments of a compound of formula Vb-5a, Vb-5b, Vb-5c, and Vb-5d, R⁷, R⁸ are methyl. In some embodiments of a compound of formula Vb-5a, Vb-5b, Vb-5c, and Vb-5d, one of R⁷, R⁸ is H, the other is methyl.

R⁷, R⁸ may be attached to the same ring atom or to different ring atoms.

In some embodiments, the present disclosure is directed towards a compound of formula Vc:

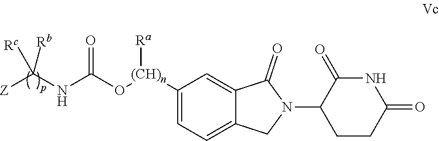

wherein
Z is H, linear or branched —C₁₋₆ alkyl, —C₃₋₆ cycloalkyl, —C₁₋₄ alkoxy, 4-8 membered heterocycloalkyl, wherein Z is unsubstituted or substituted with C₁₋₄ alkyl, C₆ aryl, C₆ aryloxy, 6 membered heteroaryl or CF₃;
or Z together with the N atom of the carbamate forms a 4-8 membered heterocycloalkyl, which is unsubstituted or substituted with C₁₋₄ alkyl, C₆ aryl, C₆ aryloxy, 6 membered heteroaryl or CF₃;
R^{a} is H, linear or branched C₁₋₄ alkyl, such as methyl; R^{b}, R^{c} are independently of each other H, linear or branched C₁₋₄ alkyl, such as methyl;
n is 1, or 2; p is 0 or 1.

In some embodiments of a compound of formula Vc, n is 1. In some embodiments of a compound of formula Vc, n is 1 and R^{a} is H. In some embodiments of a compound of formula Vc, n is 1 and R^{a} is methyl.

In some embodiments of a compound of formula Vc, p is 0. In some embodiments of a compound of formula Vc, p is 1. In some embodiments of a compound of formula Vc, p is 1, and R^{b} and R^{c} are H. In some embodiments of a compound of formula Vc, p is 1, R^{b} is methyl and R^{c} is H.

In some embodiments of a compound of formula Vc, n is 1 and p is 1. In some embodiments of a compound of formula Vc, n is 1, p is 1 and R^{a} is H. In some embodiments of a compound of formula Vc, n is 1, p is 1 and R^{a} is methyl.

In some embodiments of a compound of formula Vc, n is 1 and p is 0. In some embodiments of a compound of formula Vc, n is 1, p is 0 and R^{a} is H. In some embodiments of a compound of formula Vc, n is 1, p is 0 and R^{a} is methyl.

In some embodiments of a compound of formula Vc, Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula Vc, Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, C₆ aryl, C₆ aryloxy, 6 membered heteroaryl or CF₃.

In some embodiments of a compound of formula Vc, Z is linear or branched C₁₋₆ alkyl, C₃₋₆ cycloalkyl, pyrrolidinyl, piperdinyl, wherein Z is unsubstituted or substituted with C₁₋₄ alkyl, phenyl, phenoxy, pyridinyl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, phenyl, phenoxy, pyridinyl or CF₃.

In some embodiments of a compound of formula Vc, n is 1 and p is 1. Thus, in some embodiments a compound of formula Vc is a compound or a pharmaceutically acceptable salt or stereoisomer thereof of formula Vc-1 or Vc-1a

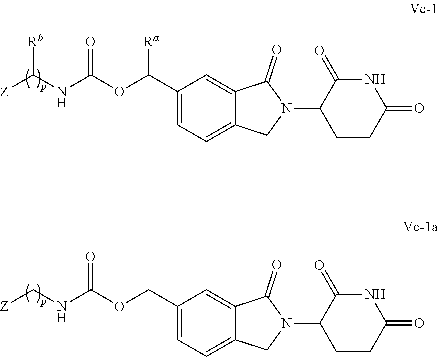

wherein
Z is H, linear or branched —C₁₋₆ alkyl, —C₃₋₆ cycloalkyl, —C₁₋₄ alkoxy, 4-8 membered heterocycloalkyl, wherein Z is unsubstituted or substituted with C₁₋₄ alkyl, C₆ aryl, C₆ aryloxy, 6 membered heteroaryl or CF₃;
or Z together with the N atom of the carbamate forms a 4-8 membered heterocycloalkyl, which is unsubstituted or substituted with C₁₋₄ alkyl, C₆ aryl, C₆ aryloxy, 6 membered heteroaryl or CF₃;
R^{a}, R^{b} are independently of each other H, linear or branched C₁₋₄ alkyl, such as methyl; and p is 0 or 1.

In some embodiments of a compound of formula Vc-1, R^{a} is H. In some embodiments of a compound of formula Vc-1, R^{a} is methyl.

In some embodiments of a compound of formula Vc-1, p is 0. In some embodiments of a compound of formula Vc-1, p is 0 and R^{a} is H. In some embodiments of a compound of formula Vc-1, p is 0 and R^{a} is methyl.

In some embodiments of a compound of formula Vc-1, p is 1. In some embodiments of a compound of formula Vc-1, p is 1 and R^{b} is H. In some embodiments of a compound of formula Vc-1, p is 1 and R^{b} is methyl. In some embodiments of a compound of formula Vc-1, p is 1 and R^{a} and R^{b} are H. In some embodiments of a compound of formula Vc-1, p is 1, R^{a} is methyl and R^{b} is H. In some embodiments of a compound of formula Vc-1, p is 1, R^{a} and R^{b} are methyl.

In some embodiments of a compound of formula Vc-1a, p is 0. In some embodiments of a compound of formula Vc-1a, p is 1.

In some embodiments of a compound of formula Vc-1 and Vc-1a, Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula Vc-1 and Vc-1a, Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, C₆ aryl, C₆ aryloxy, 6 membered heteroaryl or CF₃.

In some embodiments of a compound of formula Vc-1 and Vc-1a, Z is linear or branched C₁₋₆ alkyl, C₃₋₆ cycloalkyl, pyrrolidinyl, piperdinyl, wherein Z is unsubstituted or substituted with C₁₋₄ alkyl, phenyl, phenoxy, pyridinyl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, phenyl, phenoxy, pyridinyl or CF₃.

In some embodiments of a compound of formula Vc, n is 1 and p is 0. Thus, in some embodiments the disclosure provides a compound or a pharmaceutically acceptable salt or stereoisomer thereof of formula Vc-2:

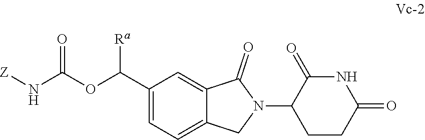

wherein
Z is —C₃₋₆ cycloalkyl, 4-8 membered heterocycloalkyl, wherein Z is unsubstituted or substituted with C₁₋₄ alkyl, C₆ aryl, C₆ aryloxy, 6 membered heteroaryl or CF₃; R^{a} is H, linear or branched C₁₋₄ alkyl, such as methyl.

In some embodiments of a compound of formula Vc-2, R^{a} is H. In some embodiments of a compound of formula Vc-2, R^{a} is methyl.

In some embodiments of a compound of formula Vc-2, Z is C₃₋₆ cycloalkyl, 4-6 membered heterocycloalkyl, wherein Z is unsubstituted or substituted with C₁₋₄ alkyl, C₆ aryl, C₆ aryloxy, 6 membered heteroaryl or CF₃.

In some embodiments of a compound of formula Vc-2, Z is C₃₋₆ cycloalkyl, 5-6 membered heterocycloalkyl, wherein Z is unsubstituted or substituted with C₁₋₄ alkyl, C₆ aryl, C₆ aryloxy, 6 membered heteroaryl or CF₃.

In some embodiments of a compound of formula Vc-2, Z is cyclopropyl, cyclobutyl, cyclopentyl, cyclohexyl, pyrrolidinyl, wherein Z is unsubstituted or substituted with linear or branched C₁₋₄ alkyl, phenyl, pyridinyl, pyrazinyl or CF₃.

In more specific embodiments, the present disclosure is directed towards a compound or a pharmaceutically acceptable salt or stereoisomer thereof of formula VI

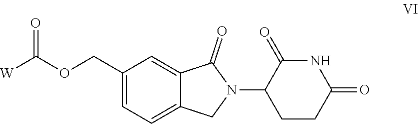

wherein W is selected from

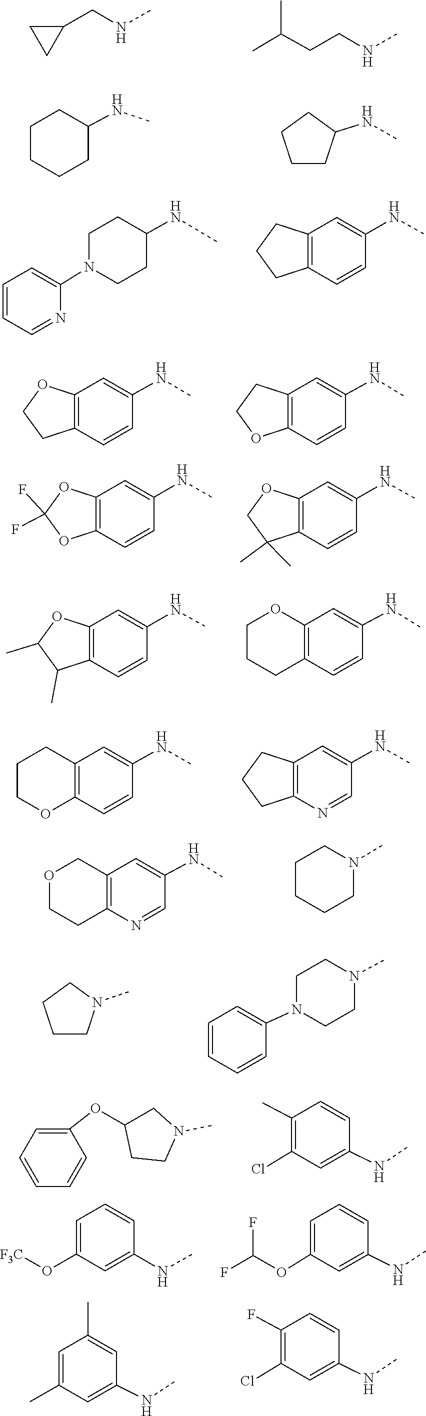

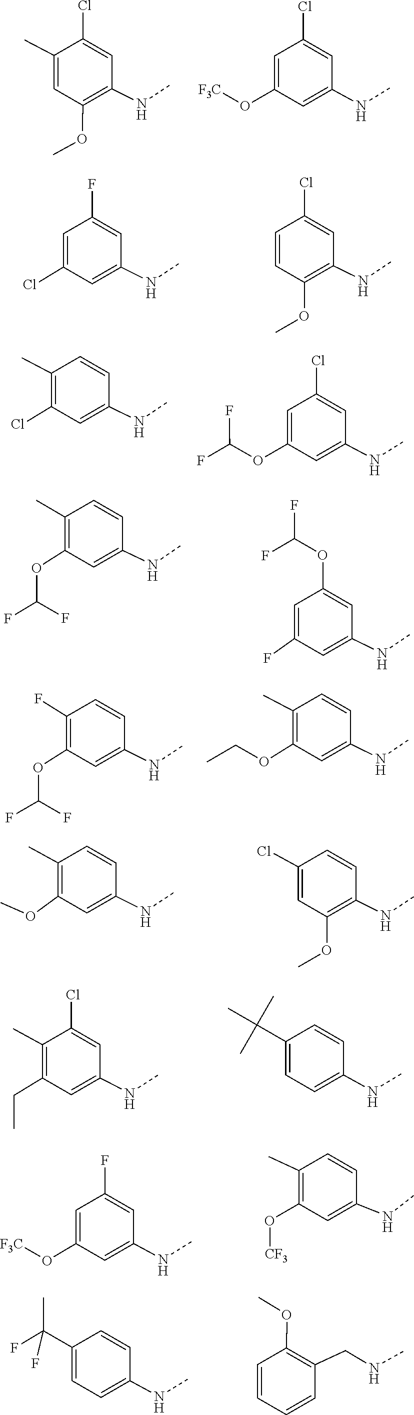

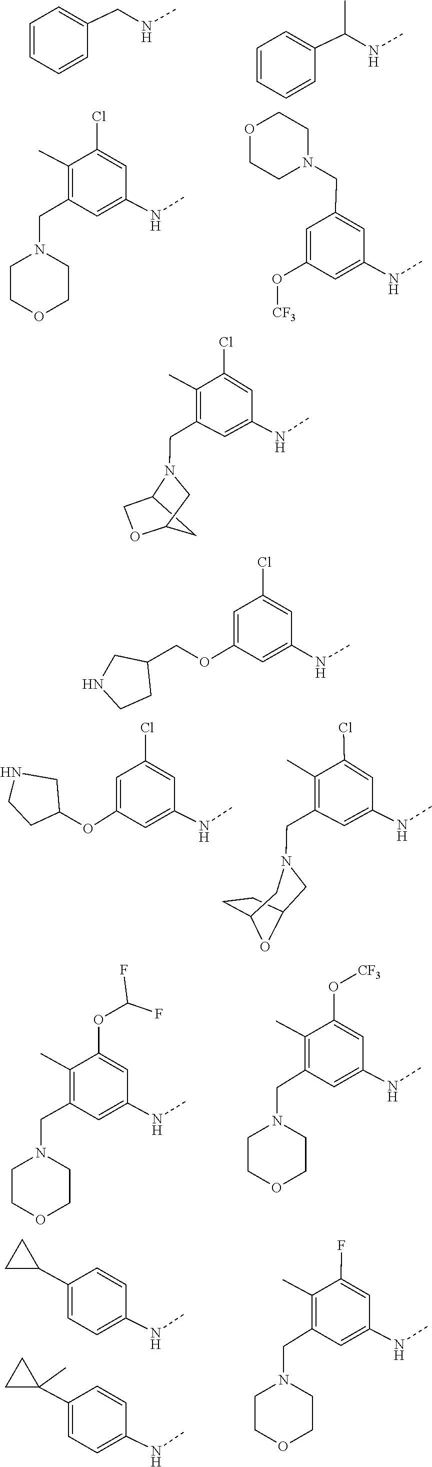

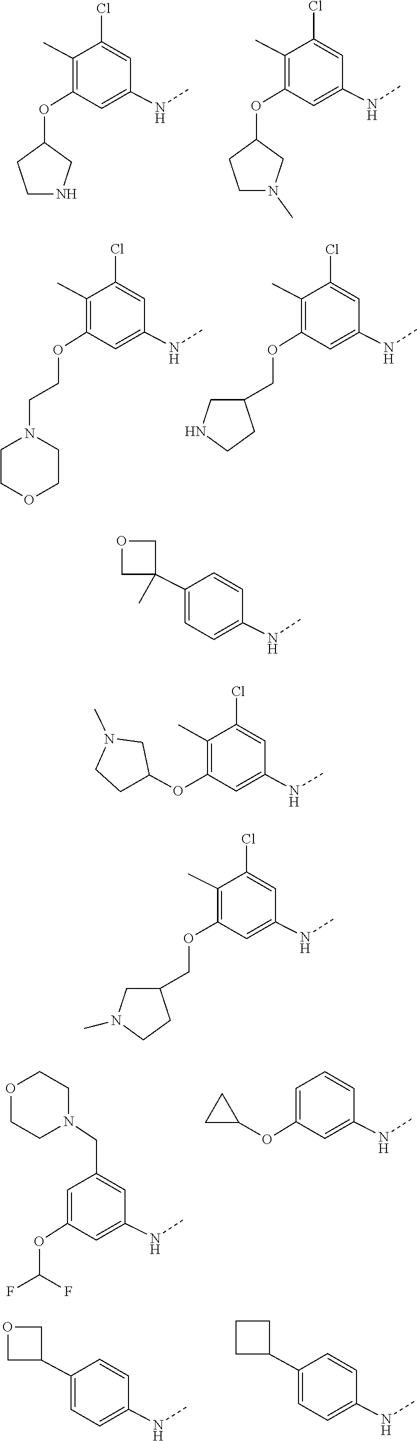

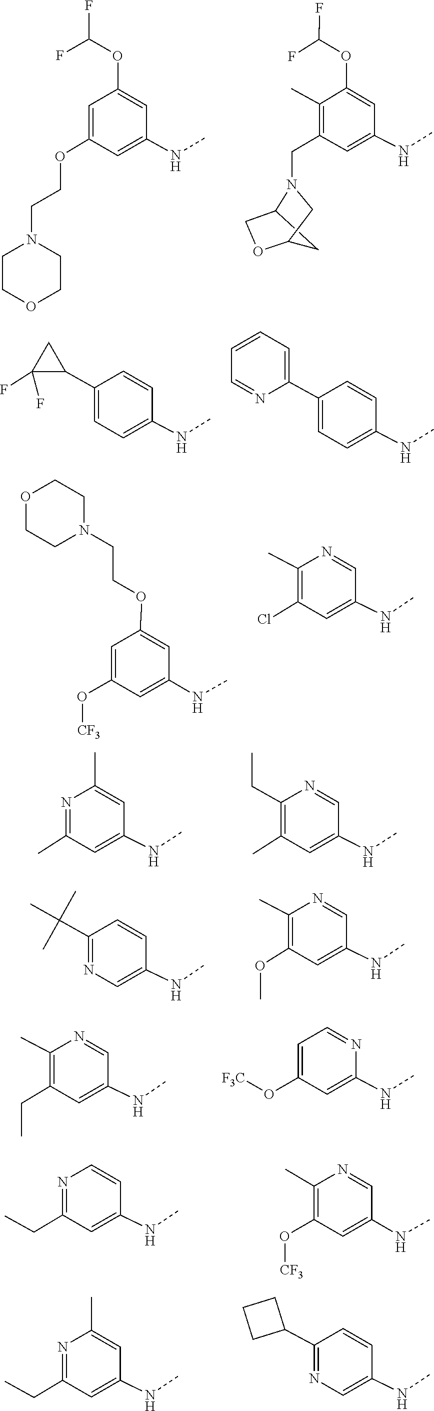

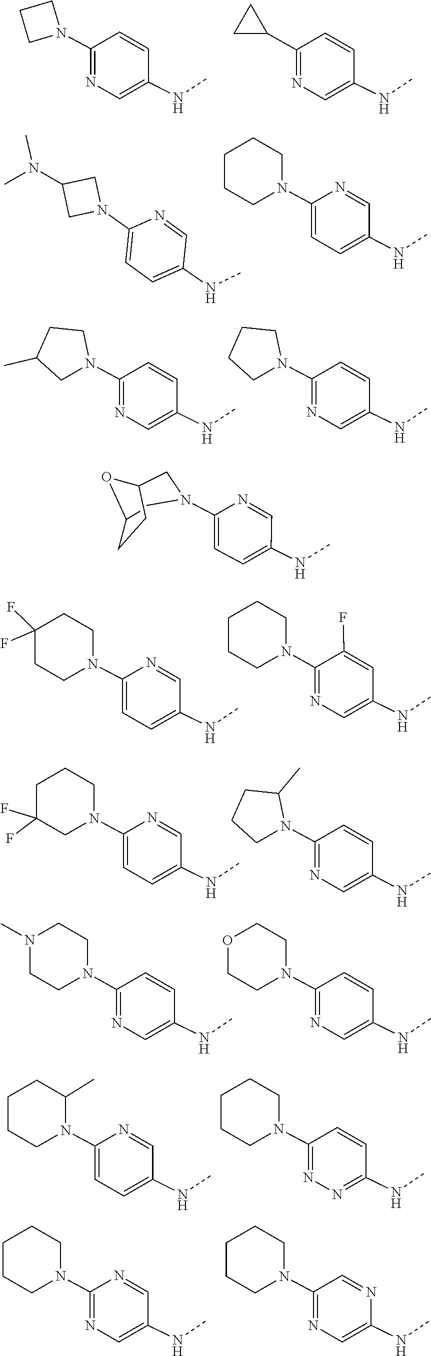

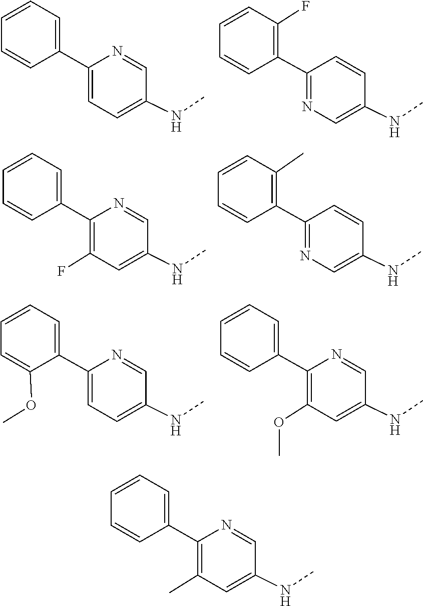

In some embodiments, the disclosure is directed to the specific examples disclosed in Tables 1, 2 and 3.

In some embodiments, the present disclosure is directed towards a compound or pharmaceutically acceptable salts or stereoisomers thereof of formula VII or VIIa, VIIb, VIIc

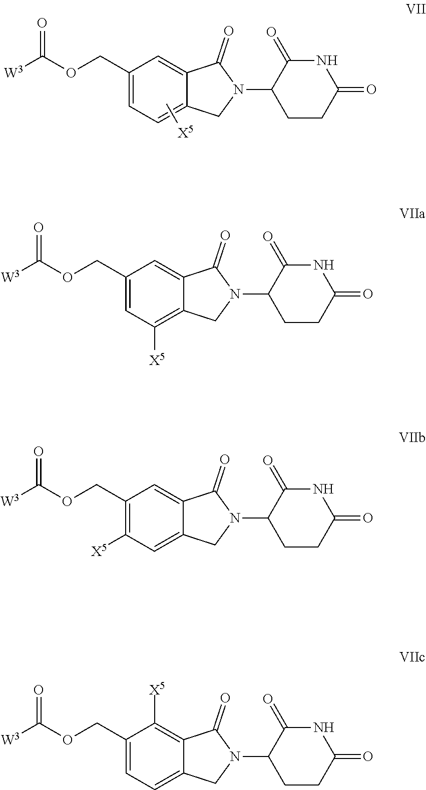

wherein X⁵ is linear or branched C₁₋₆ alkyl, —C₁₋₄ alkoxy, —CN, halogen, CF₃, CHF₂, CMeF₂, OCF₃, OCHF₂, in particular C₁₋₄ alkyl, such as methyl, —C₁₋₄ alkoxy, such as —OMe, —CN, halogen, such as F, Cl, Br; and W³ is selected from

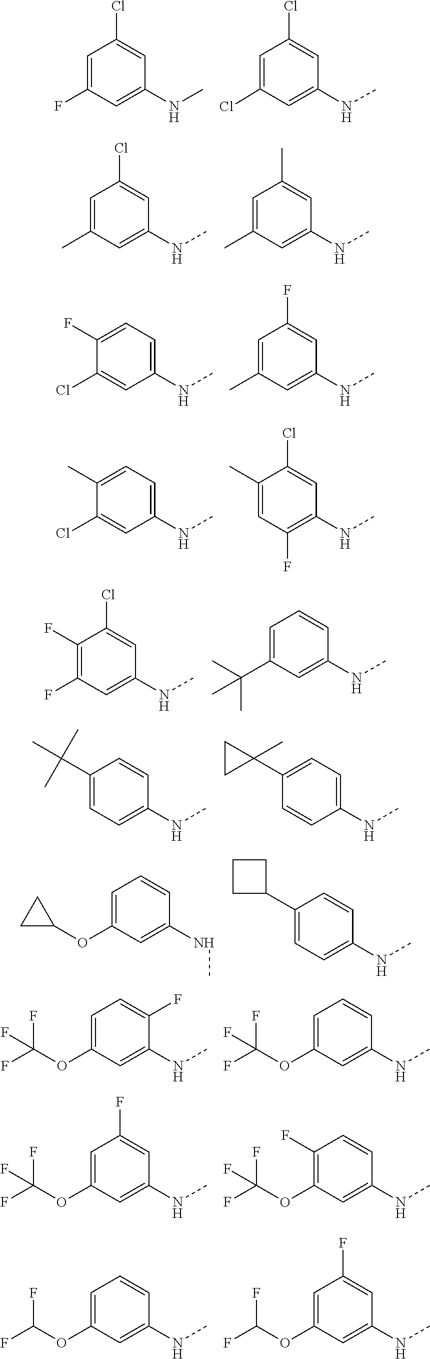

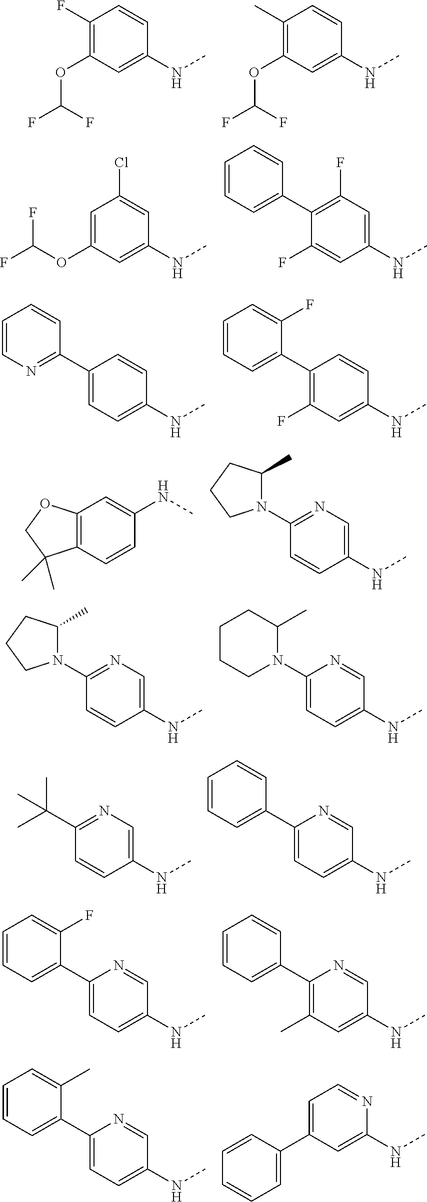

In some embodiments, the disclosure is directed to the (S) enantiomer of the compounds of any of formula I-VII. In some embodiments, the disclosure is directed to the (R) enantiomer of the compounds of any of formula I-VII. In some embodiments, the disclosure is directed to the racemate of the compounds of any of formula I-VII.

The compounds of the disclosure may contain one or more asymmetric centers in the molecule. A compound without designation of the stereochemistry is to be understood to include all the optical isomers (e.g., diastereomers, enantiomers, etc.) in pure or substantially pure form, as well as mixtures thereof (e.g. a racemic mixture, or an enantiomerically enriched mixture). It is well known in the art how to prepare such optically active forms (e.g. by resolution of the racemic form by recrystallization techniques, by synthesis from optically-active starting materials, by chiral synthesis, by chromatographic separation using a chiral stationary phase, and other methods).

The compounds may be isotopically-labeled compounds, for example, compounds including various isotopes of hydrogen, carbon, nitrogen, oxygen, phosphorous, fluorine, iodine, or chlorine. The disclosed compounds may exist in tautomeric forms and mixtures and separate individual tautomers are contemplated. In addition, some compounds may exhibit polymorphism.

The compounds of the disclosure include the free form as well as a pharmaceutically acceptable salt or stereoisomer thereof. The pharmaceutically acceptable salts include all the typical pharmaceutically acceptable salts. The pharmaceutically acceptable salts of the present compounds can be synthesized from the compounds of this disclosure which contain a basic or acidic moiety by conventional chemical methods, see e.g. Berge et al, “Pharmaceutical Salts,” J. Pharm. ScL, 1977:66:1-19. Furthermore, the compounds of the disclosure also include lyophilized and polymorphs of the free form.

For example, conventional pharmaceutically acceptable salts for a basic compound include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional pharmaceutically acceptable salts for an acidic compound include those derived from inorganic bases include aluminum, ammonium, calcium, copper, ferric, ferrous, lithium, magnesium, manganic salts, manganous, potassium, sodium, zinc and the like. Salts derived from pharmaceutically acceptable organic bases include salts of primary, secondary and tertiary amines, substituted amines including naturally occurring substituted amines, cyclic amines and basic ion exchange resins, such as arginine, betaine caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The compounds of the disclosure may exist in solid, i.e. crystalline or noncrystalline form (optionally as solvates) or liquid form. In the solid state, it may exist in, or as a mixture thereof. In crystalline solvates, solvent molecules are incorporated into the crystalline lattice during crystallization. The formation of solvates may include non-aqueous solvents such as, but not limited to, ethanol, isopropanol, DMSO, acetic acid, ethanolamine, or ethyl acetate, or aqueous solvents such as water (also called “hydrates”). It is common knowledge that crystalline forms (and solvates thereof) may exhibit polymorphism, i.e. exist in different crystalline structures known as “polymorphs”, that have the same chemical composition but differ in packing, geometrical arrangement, and other descriptive properties of the crystalline solid state. Polymorphs, therefore, may have different physical properties such as shape, density, hardness, deformability, stability, and dissolution properties, and may display different melting points, IR spectra, and X-ray powder diffraction patterns, which may be used for identification. Such different polymorphs may be produced, for example, by changing or adjusting the reaction conditions or reagents, during preparation of the compound of the disclosure.

In a further aspect, the disclosure also provides methods of preparation of the compounds of formula I-VII of the disclosure. In some embodiments, they are prepared according to the general procedure A.

In yet another aspect, the disclosure further provides a pharmaceutical composition comprising a therapeutically-effective amount of one or more of the compounds of the disclosure a pharmaceutically acceptable salt or stereoisomer thereof and one or more pharmaceutically acceptable carriers and/or excipients (also referred to as diluents). The excipients are acceptable in the sense of being compatible with the other ingredients of the formulation and not deleterious to the recipient thereof (i.e., the patient). The term “therapeutically-effective amount” as used herein refers to the amount of a compound (as such or in form of a pharmaceutical composition) of the present disclosure which is effective for producing some desired therapeutic effect.

Pharmaceutical compositions may be in unit dose form containing a predetermined amount of a compound of the disclosure per unit dose. Such a unit may contain a therapeutically effective dose of a compound of the disclosure a pharmaceutically acceptable salt or stereoisomer thereof or a fraction of a therapeutically effective dose such that multiple unit dosage forms might be administered at a given time to achieve the desired therapeutically effective dose. Preferred unit dosage formulations are those containing a daily dose or sub-dose, or an appropriate fraction thereof, of a compound of the disclosure a pharmaceutically acceptable salt or stereoisomer thereof.

The compounds of the disclosure may be administered by any acceptable means in solid or liquid form, including (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; (9) pulmonary; or (10) intrathecally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical compositions.

Such compositions may contain further components conventional in pharmaceutical preparations, e.g. wetting agents, emulsifiers and lubricants, such as sodium lauryl sulfate and magnesium stearate, as well as coloring agents, release agents, coating agents, sweetening, flavoring and perfuming agents, preservatives and antioxidants, pH modifiers, bulking agents, and further active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediaminetetraacetic acid (EDTA), sorbitol, tartaric acid, phosphoric acid, and the like.

Such compositions may be prepared by any method known in the art, for example, by bringing into association the active ingredient with one or more carriers and/or excipients. Different compositions and examples of carriers and/or excipients are well known to the skilled person and are described in detail in, e.g., Remington: The Science and Practice of Pharmacy. Pharmaceutical Press, 2013; Rowe, Sheskey, Quinn: Handbook of Pharmaceutical Excipients. Pharmaceutical Press, 2009. Excipients that may be used in the preparation of the pharmaceutical compositions may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

As indicated above, the compounds of the present disclosure may be in solid or liquid form and administered by various routes in any convenient administrative form, e.g., tablets, powders, capsules, solutions, dispersions, suspensions, syrups, sprays, suppositories, gels, emulsions, patches, etc.

In solid dosage forms of the disclosure for oral administration (capsules, tablets, pills, dragees, powders, granules, trouches and the like), a compound is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. An oral composition can also include adjuvants such as wetting agents, emulsifying and suspending agents, sweetening, flavoring, coloring, perfuming and preservative agents.

In form of suspensions, a compound may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for rectal or vaginal administration of a compound of the disclosure include a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Other suitable forms include pessaries, tampons, creams, gels, pastes, foams or spray formulations containing such carriers as are known in the art to be appropriate.

Dosage forms for the topical or transdermal administration of a compound of the disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. Such ointments, pastes, creams and gels may contain, in addition to a compound of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Dosage forms such as powders and sprays for administration of a compound of the disclosure, may contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms such as transdermal patches for administration of a compound of the disclosure may include absorption enhancers or retarders to increase or decrease the flux of the compound across the skin. The rate of such flux can be controlled by either providing a rate controlling membrane or dispersing the compound in a polymer matrix or gel. Other dosage forms contemplated include ophthalmic formulations, eye ointments, powders, solutions and the like. It is understood that all contemplated compositions must be stable under the conditions of manufacture and storage, and preserved against the contaminating action of microorganisms, such as bacteria and fungi.

The dosage levels of a compound of the disclosure in the pharmaceutical compositions of the disclosure may be adjusted in order to obtain an amount of a compound of the disclosure which is effective to achieve the desired therapeutic response for a particular patient, composition, and mode of administration, without being deleterious to the patient. The dosage of choice will depend upon a variety of factors including the nature of the particular compound of the present disclosure used, the route of administration, the time of administration, the rate of excretion or metabolism of the particular compound used, the rate and extent of absorption, the duration of the treatment, other drugs, compounds and/or materials used in combination with the particular compound, the age, sex, weight, condition, general health and prior medical history of the patient being treated, and like factors well known in the medical arts. A medical practitioner having ordinary skill in the art can readily determine and prescribe the effective amount of the pharmaceutical composition required.

Typically, a suitable daily dose of a compound of the disclosure will be that amount of the compound which is the lowest dose effective to produce a therapeutic effect. Such an effective dose will generally depend upon the factors described above. Generally, oral, intravenous, intracerebroventricular and subcutaneous doses of the compounds of this disclosure for a patient, when used for the indicated analgesic effects, will range from about 0.0001 to about 100 mg, more usual 0.1 to 100 mg/kg per kilogram of body weight of recipient (patient, mammal) per day. Acceptable daily dosages may be from about 1 to about 1000 mg/day, and for example, from about 1 to about 100 mg/day.

### EXAMPLES

### Example 1: Preparation of Compounds 1 to 160 and 200-307 and 400-473

Compounds 1 to 160 were prepared as described in WO 2021/069705, hereby incorporated by reference.

TABLE 1
Specific examples 1-160

| Compound | NO |
|---|---|
| 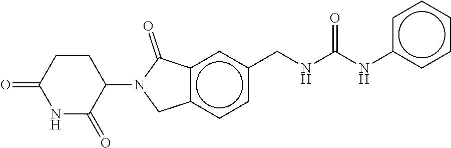 | 1 |
| 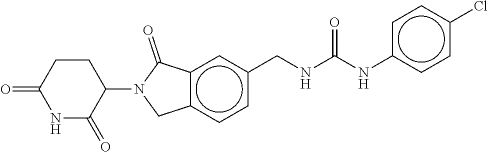 | 2 |
| 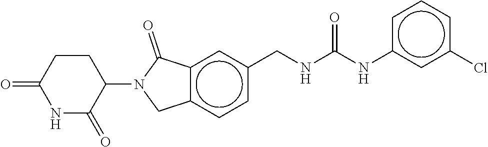 | 3 |
| 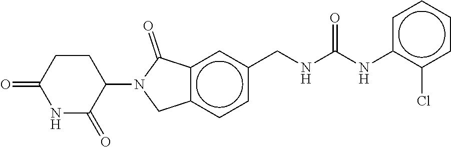 | 4 |
| 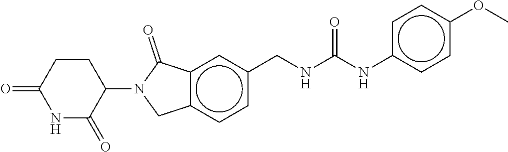 | 5 |
| 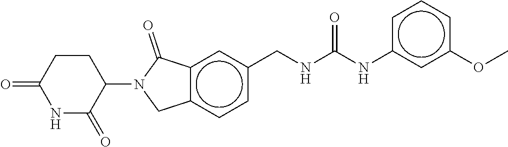 | 6 |
| 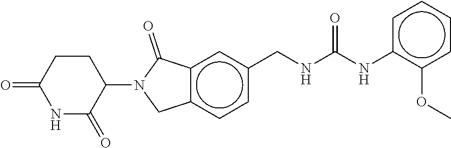 | 7 |
| 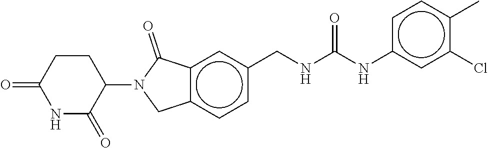 | 8 |
| 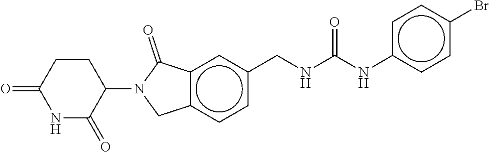 | 9 |
| 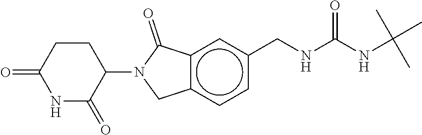 | 10 |
| 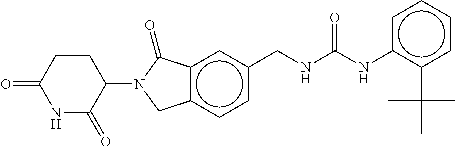 | 11 |
| 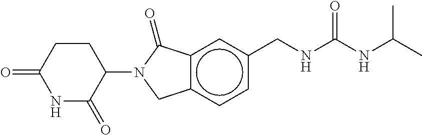 | 12 |
| 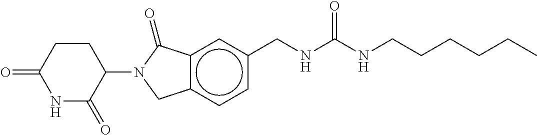 | 13 |
| 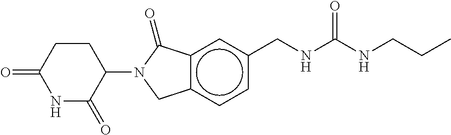 | 14 |
| 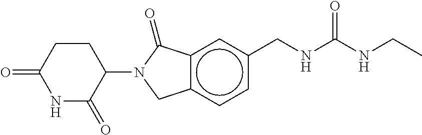 | 15 |
| 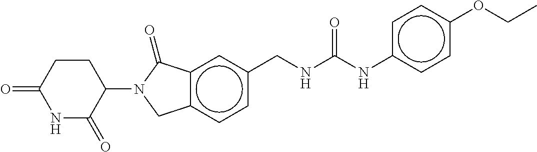 | 16 |
| 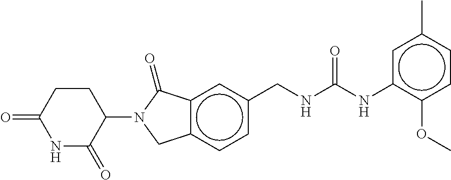 | 17 |
| 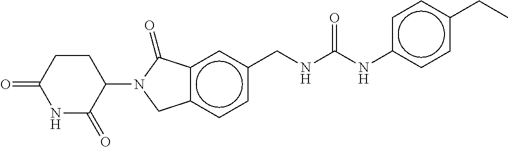 | 18 |
| 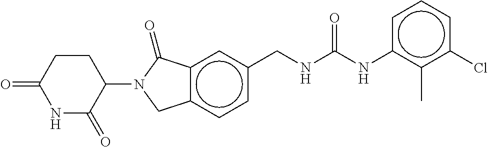 | 19 |
| 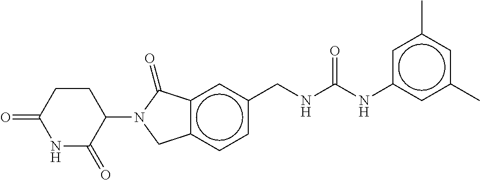 | 20 |
| 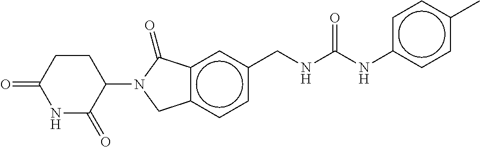 | 21 |
| 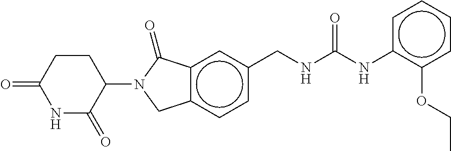 | 22 |
| 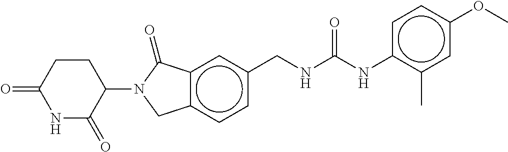 | 23 |
| 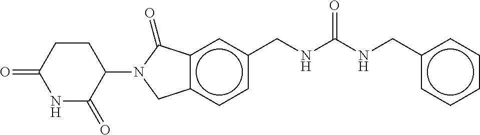 | 24 |
| 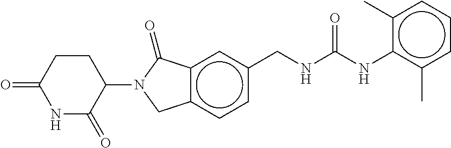 | 25 |
| 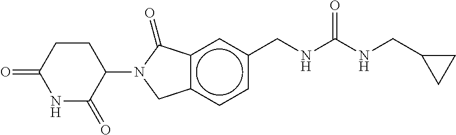 | 26 |
| 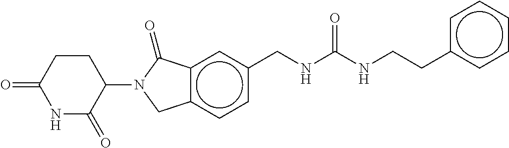 | 27 |
| 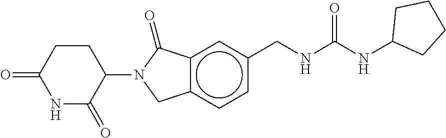 | 28 |
| 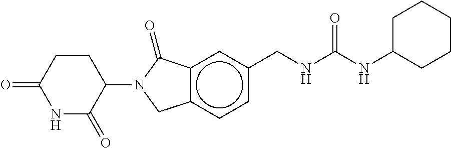 | 29 |
| 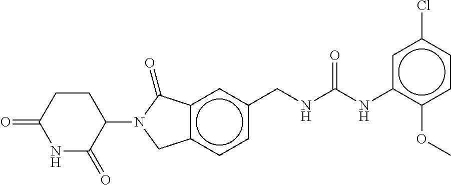 | 30 |
| 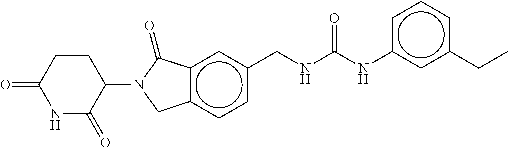 | 31 |
| 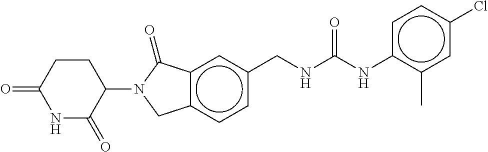 | 32 |
| 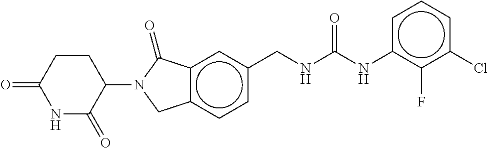 | 33 |
| 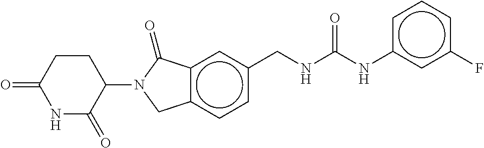 | 34 |
| 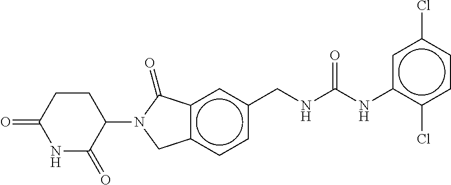 | 35 |
| 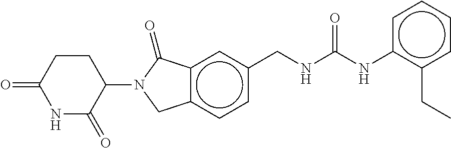 | 36 |
| 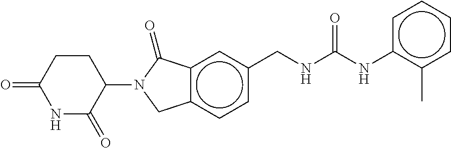 | 37 |
| 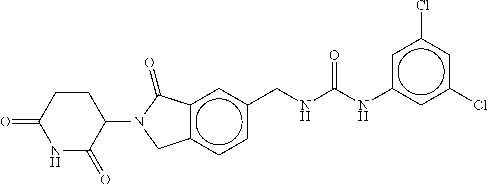 | 38 |
| 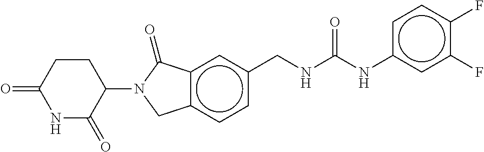 | 39 |
| 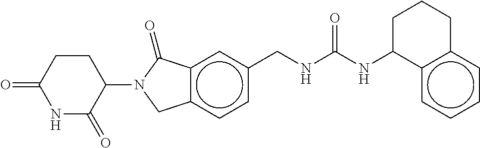 | 40 |
| 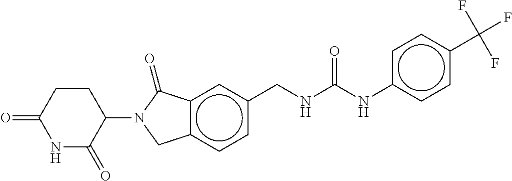 | 41 |
| 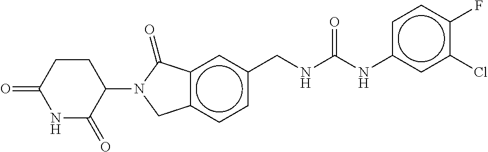 | 42 |
| 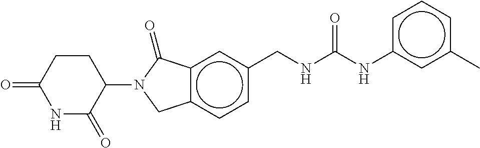 | 43 |
| 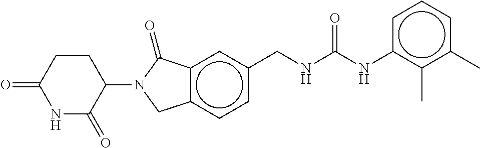 | 44 |
| 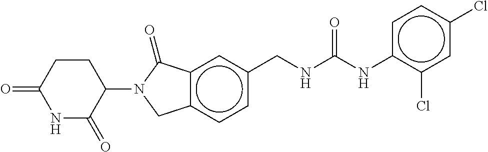 | 45 |
| 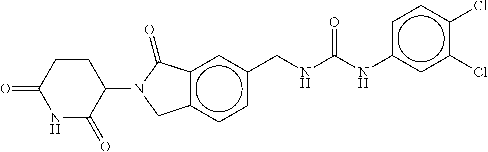 | 46 |
| 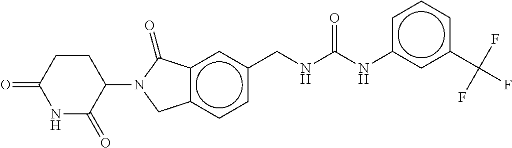 | 47 |
| 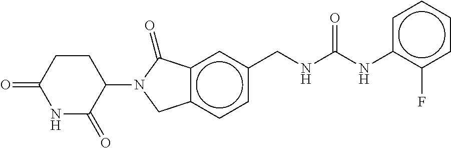 | 48 |
| 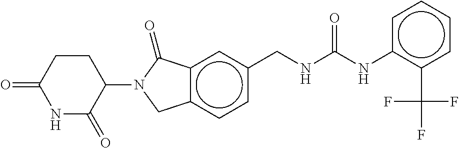 | 49 |
| 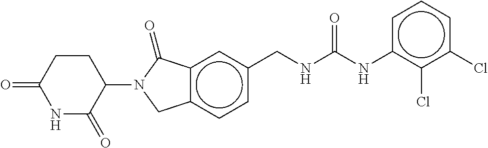 | 50 |
| 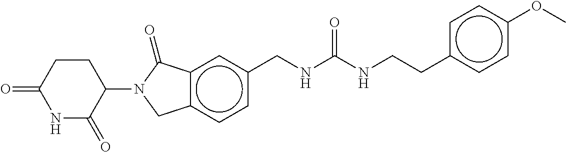 | 51 |
| 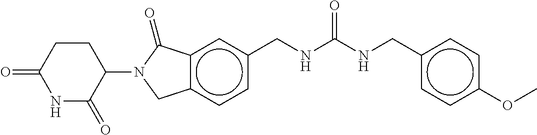 | 52 |
| 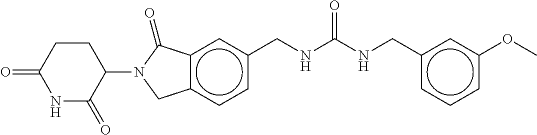 | 53 |
| 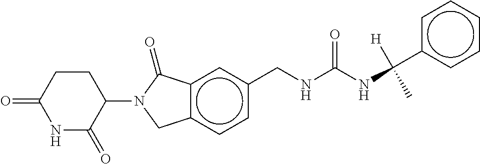 | 54 |
| 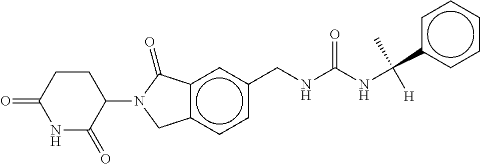 | 55 |
| 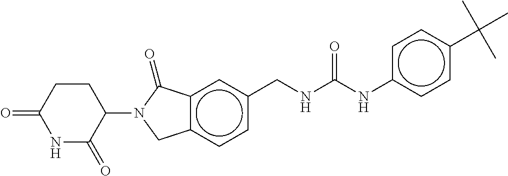 | 56 |
| 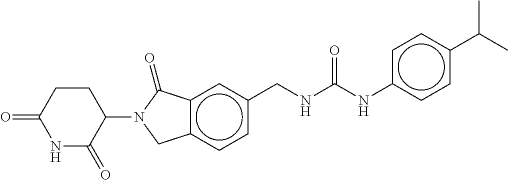 | 57 |
| 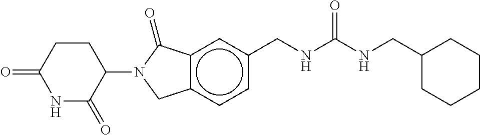 | 58 |
| 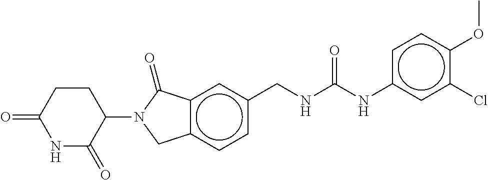 | 59 |
| 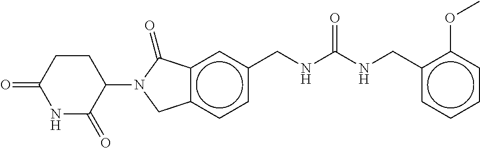 | 60 |
| 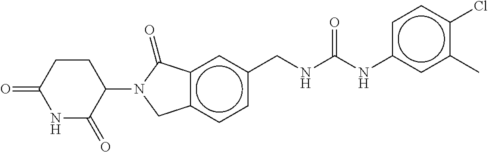 | 61 |
| 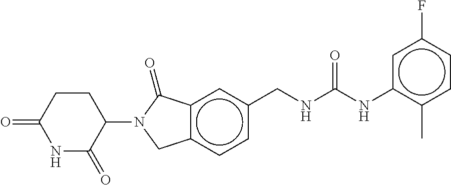 | 62 |
| 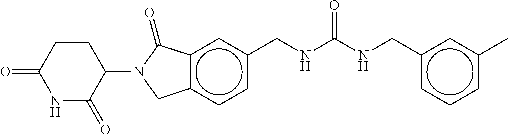 | 63 |
| 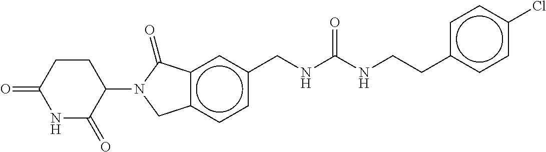 | 64 |
| 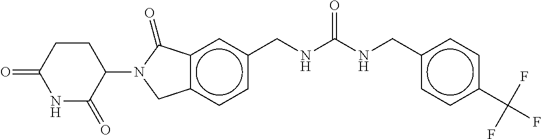 | 65 |
| 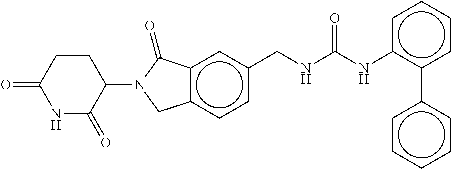 | 66 |
| 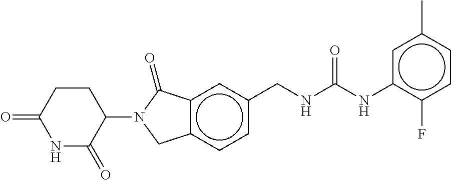 | 67 |
| 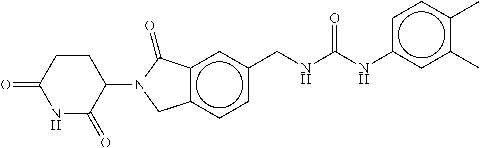 | 68 |
| 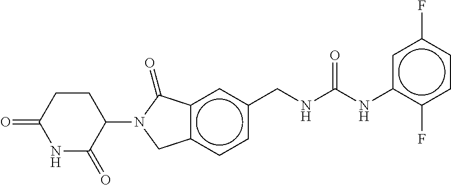 | 69 |
| 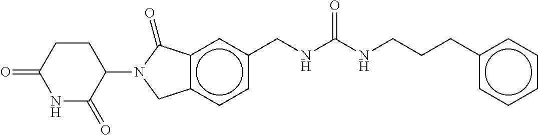 | 70 |
| 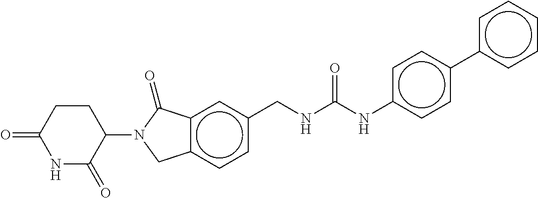 | 71 |
| 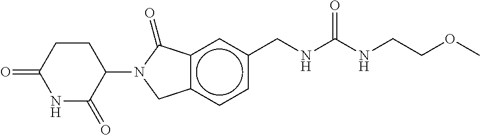 | 72 |
| 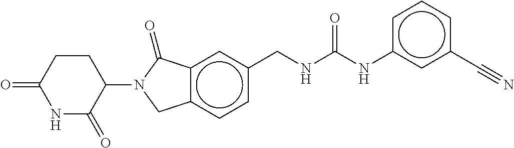 | 73 |
| 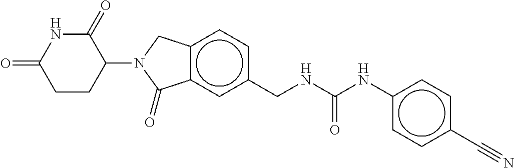 | 74 |
| 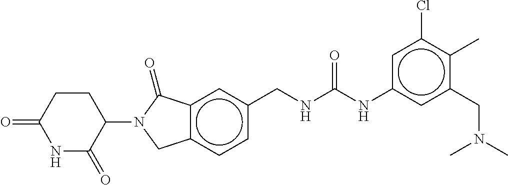 | 75 |
| 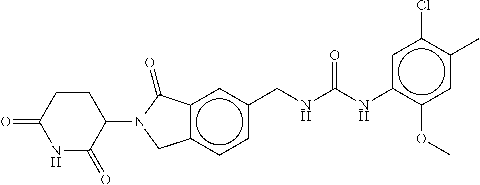 | 76 |
| 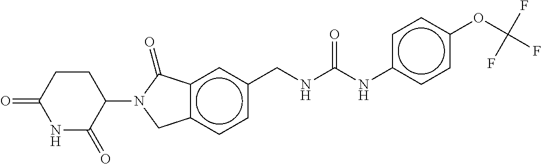 | 77 |
| 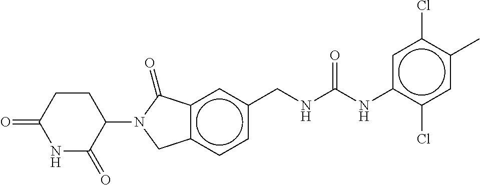 | 78 |
| 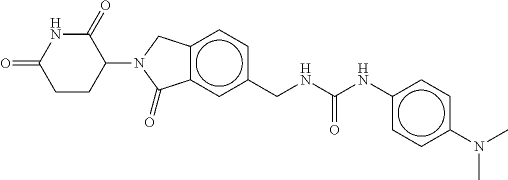 | 79 |
| 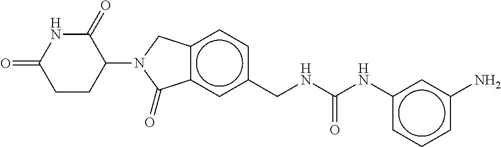 | 80 |
| 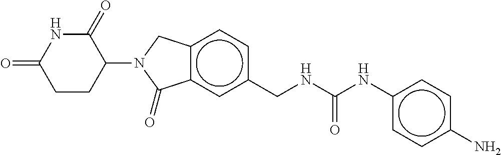 | 81 |
| 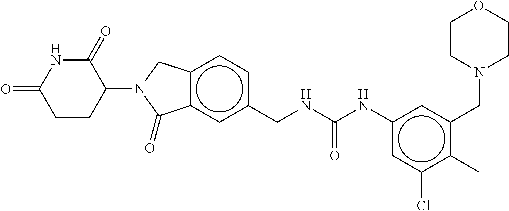 | 82 |
| 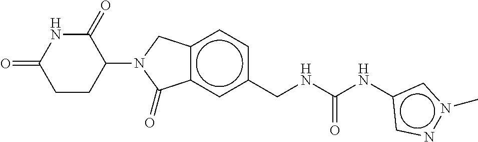 | 83 |
| 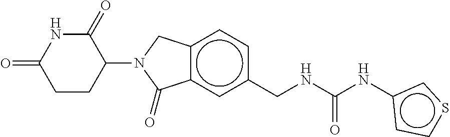 | 84 |
| 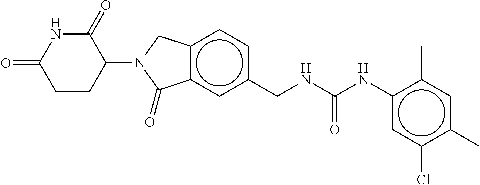 | 85 |
| 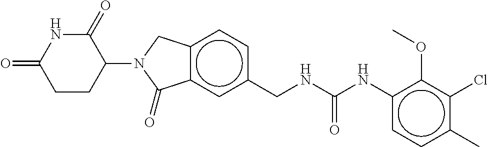 | 86 |
| 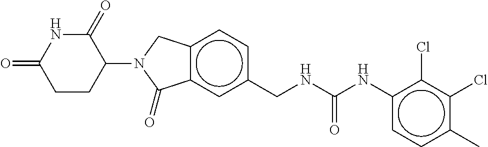 | 87 |
| 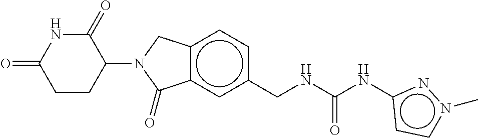 | 88 |
| 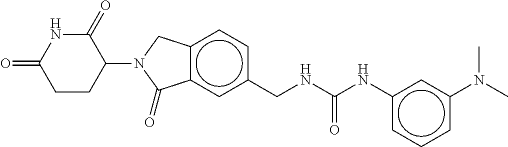 | 89 |
| 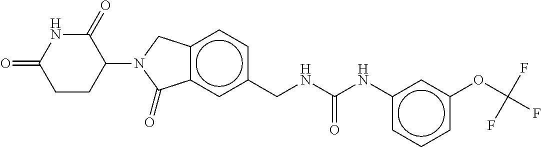 | 90 |
| 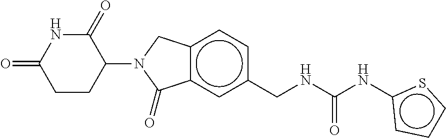 | 91 |
| 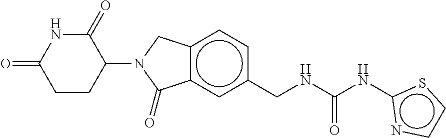 | 92 |
| 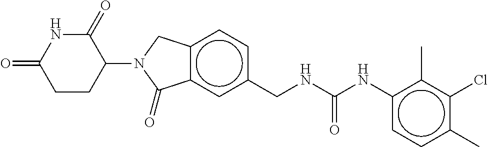 | 93 |
| 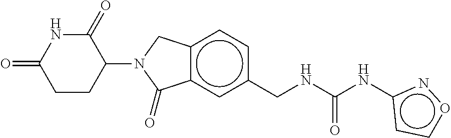 | 94 |
| 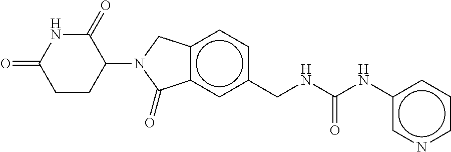 | 95 |
| 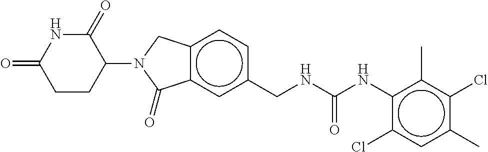 | 96 |
| 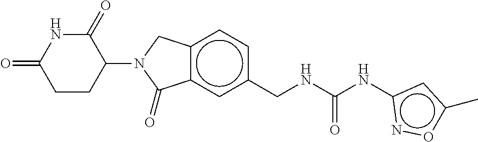 | 97 |
| 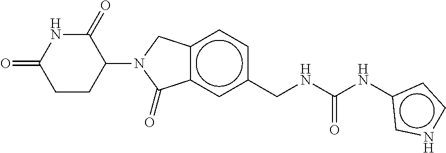 | 98 |
| 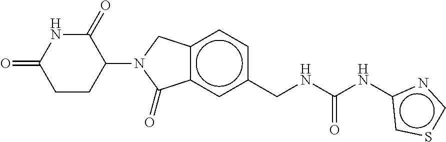 | 99 |
| 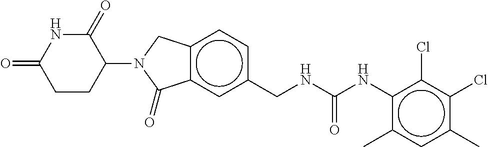 | 100 |
| 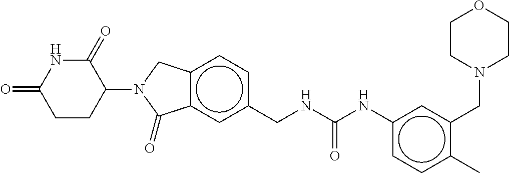 | 101 |
| 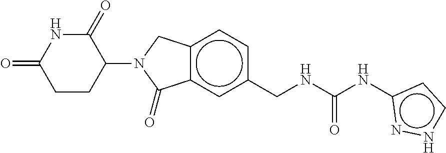 | 102 |
| 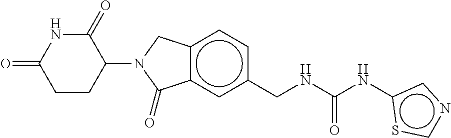 | 103 |
| 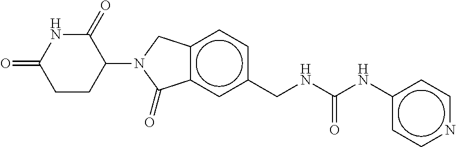 | 104 |
| 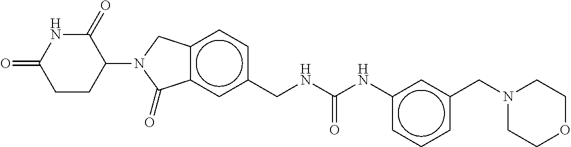 | 105 |
| 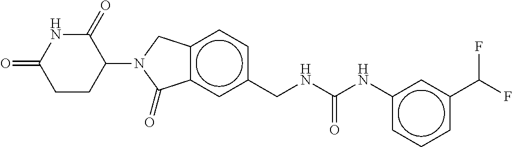 | 106 |
| 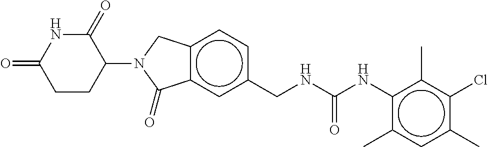 | 107 |
| 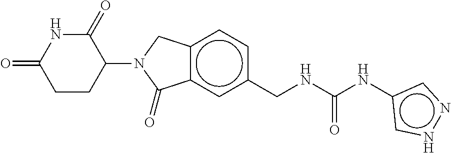 | 108 |
| 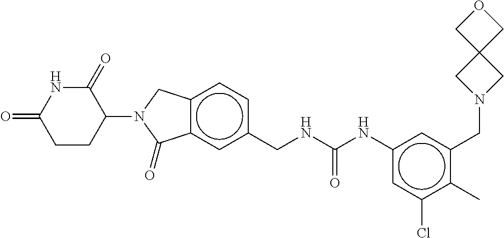 | 109 |
| 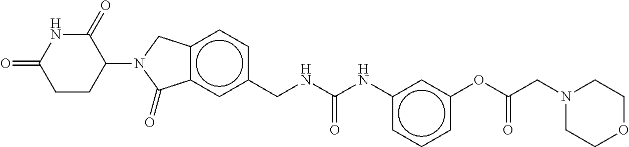 | 110 |
| 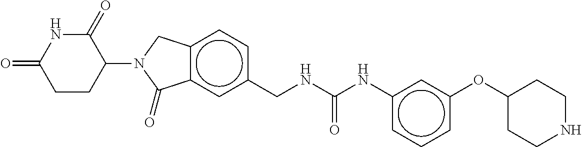 | 111 |
| 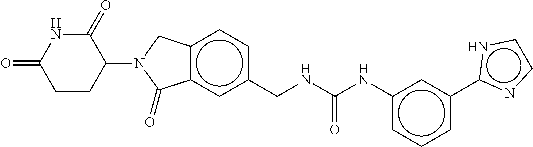 | 112 |
| 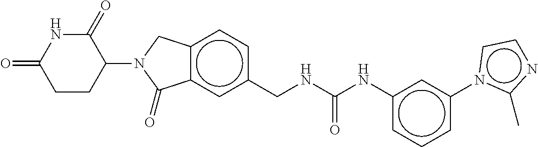 | 113 |
| 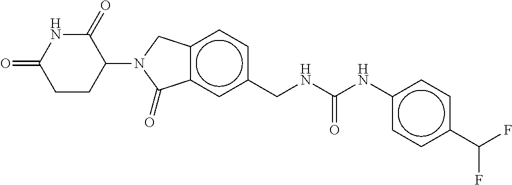 | 114 |
| 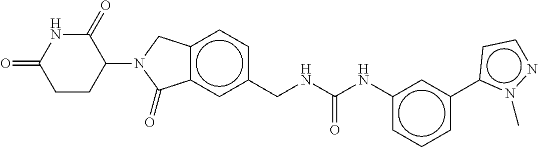 | 115 |
| 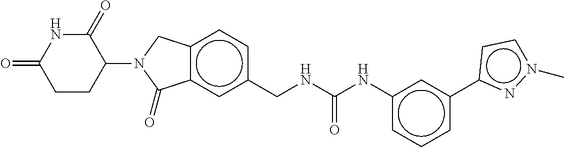 | 116 |
| 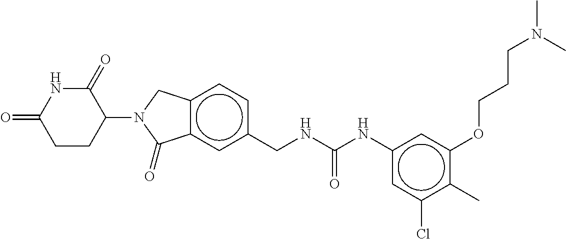 | 117 |
| 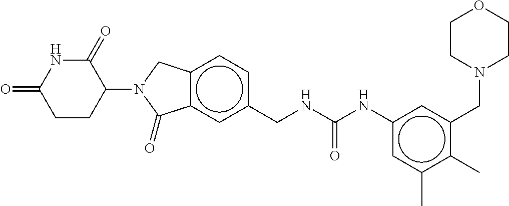 | 118 |
| 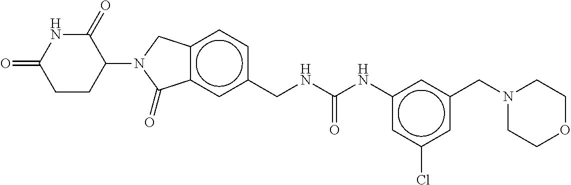 | 119 |
| 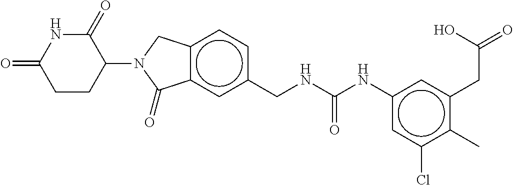 | 120 |
| 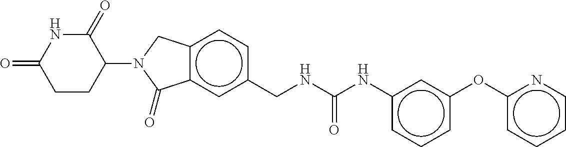 | 121 |
| 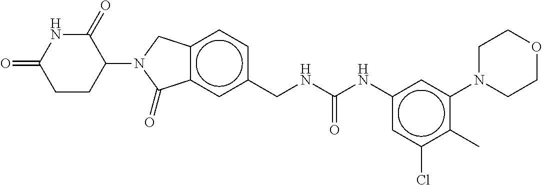 | 122 |
| 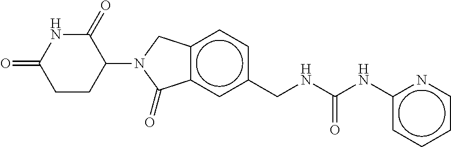 | 123 |
| 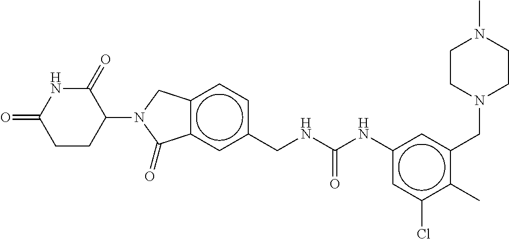 | 124 |
| 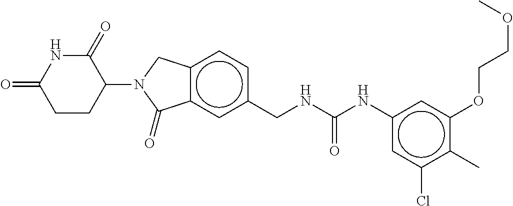 | 125 |
| 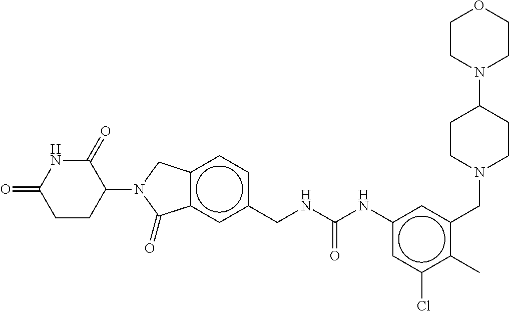 | 126 |
| 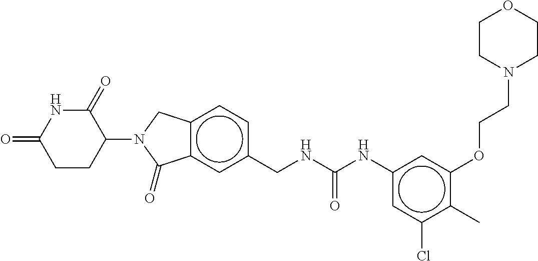 | 127 |
| 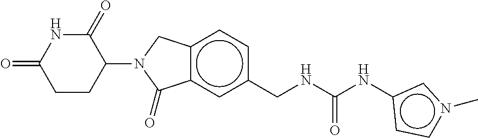 | 128 |
| 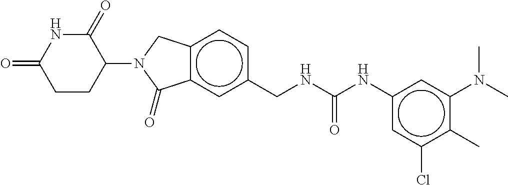 | 129 |
| 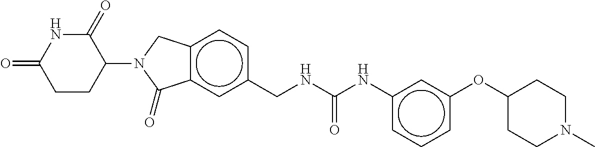 | 130 |
| 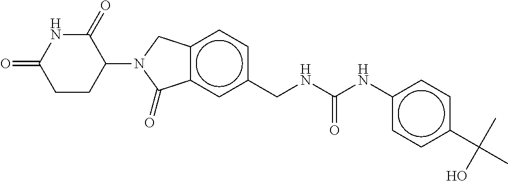 | 131 |
| 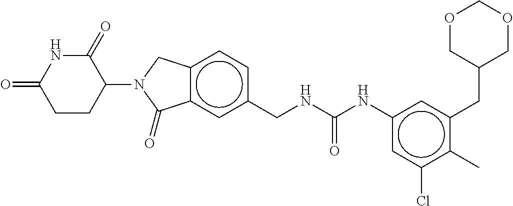 | 132 |
| 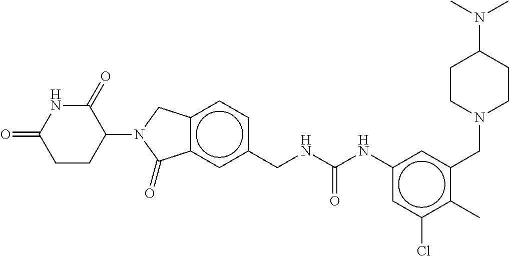 | 133 |
| 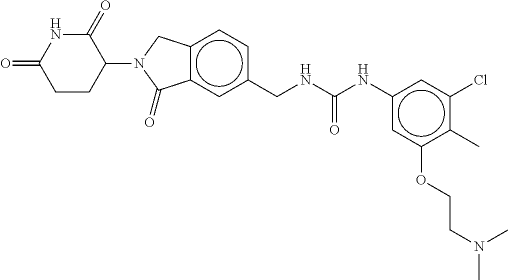 | 134 |
| 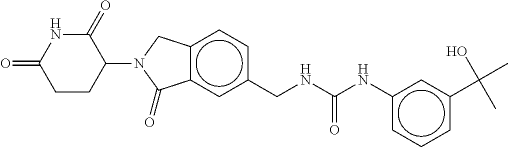 | 135 |
| 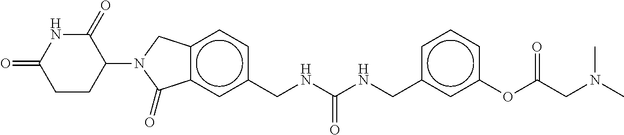 | 136 |
| 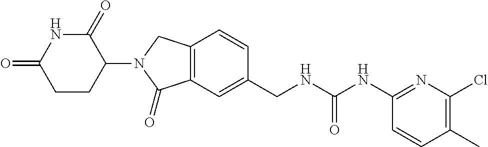 [SMILES: Cc1ccc(NC(=O)NCc2ccc3c(c2)C(=O)N(C2CCC(=O)NC2=O)C3)nc1Cl] | 137 |
| 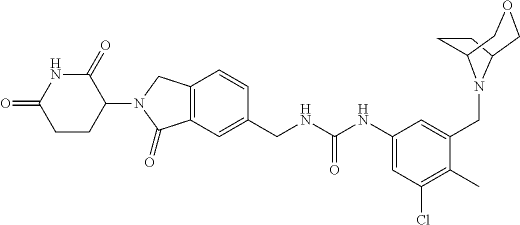 [SMILES: Cc1c(Cl)cc(NC(=O)NCc2ccc3c(c2)C(=O)N(C2CCC(=O)NC2=O)C3)cc1CN1C2CCC1COC2] | 138 |
| 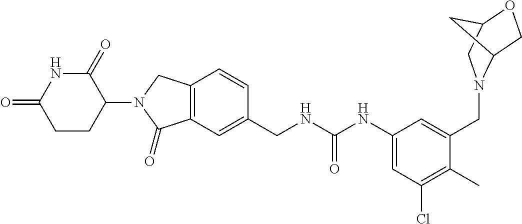 [SMILES: Cc1c(Cl)cc(NC(=O)NCc2ccc3c(c2)C(=O)N(C2CCC(=O)NC2=O)C3)cc1CN1CC2CC1CO2] | 139 |
| 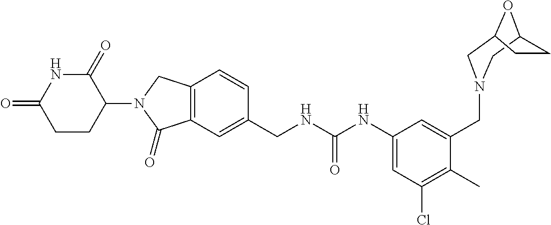 [SMILES: Cc1c(Cl)cc(NC(=O)NCc2ccc3c(c2)C(=O)N(C2CCC(=O)NC2=O)C3)cc1CN1CC2CCC(C1)O2] | 140 |
| 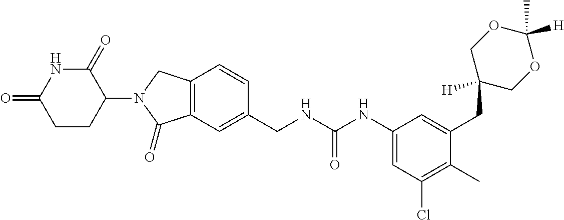 [SMILES: Cc1c(Cl)cc(NC(=O)NCc2ccc3c(c2)C(=O)N(C2CCC(=O)NC2=O)C3)cc1C[C@H]1CO[C@H](C)OC1] | 141 |
| 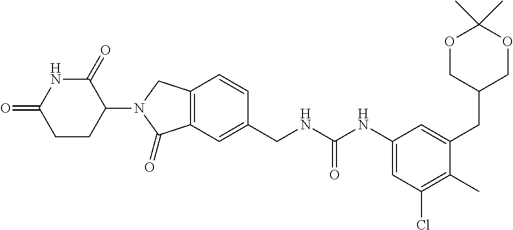 [SMILES: Cc1c(Cl)cc(NC(=O)NCc2ccc3c(c2)C(=O)N(C2CCC(=O)NC2=O)C3)cc1CC1COC(C)(C)OC1] | 142 |
| 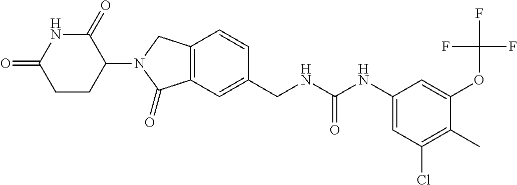 [SMILES: Cc1c(Cl)cc(NC(=O)NCc2ccc3c(c2)C(=O)N(C2CCC(=O)NC2=O)C3)cc1OC(F)(F)F] | 143 |
| 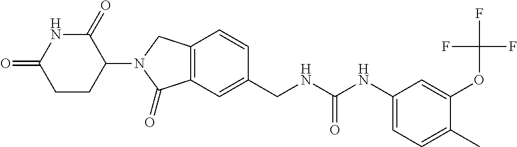 [SMILES: Cc1ccc(NC(=O)NCc2ccc3c(c2)C(=O)N(C2CCC(=O)NC2=O)C3)cc1OC(F)(F)F] | 144 |
| 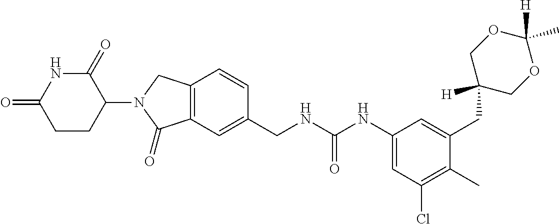 [SMILES: Cc1c(Cl)cc(NC(=O)NCc2ccc3c(c2)C(=O)N(C2CCC(=O)NC2=O)C3)cc1C[C@H]1CO[C@@H](C)OC1] | 145 |
| 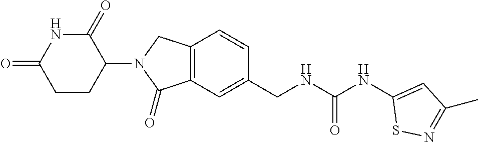 [SMILES: Cc1cc(NC(=O)NCc2ccc3c(c2)C(=O)N(C2CCC(=O)NC2=O)C3)sn1] | 146 |
| 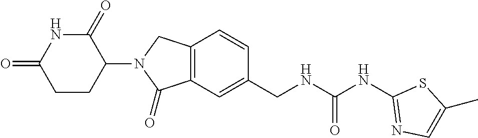 [SMILES: Cc1cnc(NC(=O)NCc2ccc3c(c2)C(=O)N(C2CCC(=O)NC2=O)C3)s1] | 147 |
| 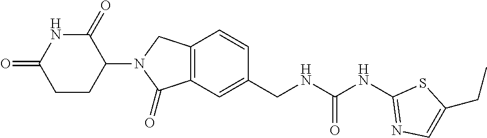 [SMILES: CCc1cnc(NC(=O)NCc2ccc3c(c2)C(=O)N(C2CCC(=O)NC2=O)C3)s1] | 148 |
| 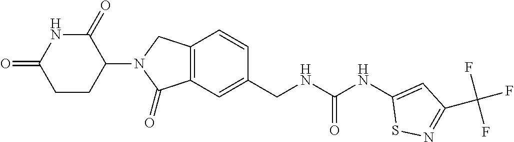 [SMILES: O=C1CCC(N2Cc3ccc(CNC(=O)Nc4cc(C(F)(F)F)ns4)cc3C2=O)C(=O)N1] | 149 |
| 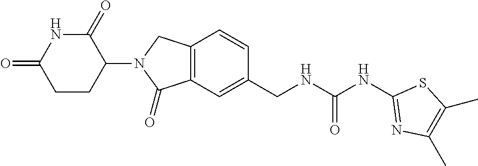 [SMILES: Cc1nc(NC(=O)NCc2ccc3c(c2)C(=O)N(C2CCC(=O)NC2=O)C3)sc1C] | 150 |
| 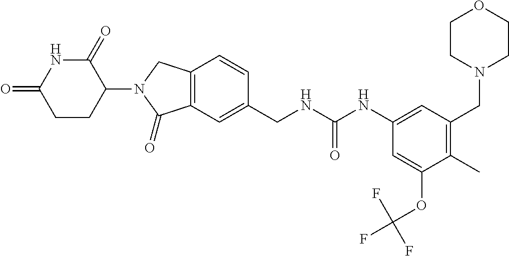 [SMILES: Cc1c(CN2CCOCC2)cc(NC(=O)NCc2ccc3c(c2)C(=O)N(C2CCC(=O)NC2=O)C3)cc1OC(F)(F)F] | 151 |
| 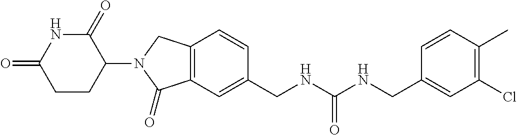 [SMILES: Cc1ccc(CNC(=O)NCc2ccc3c(c2)C(=O)N(C2CCC(=O)NC2=O)C3)cc1Cl] | 152 |
| 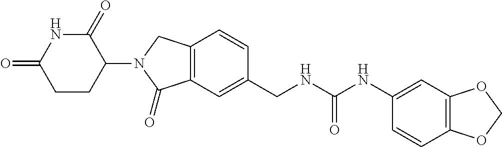 [SMILES: O=C1CCC(N2Cc3ccc(CNC(=O)Nc4ccc5c(c4)OCO5)cc3C2=O)C(=O)N1] | 153 |
| 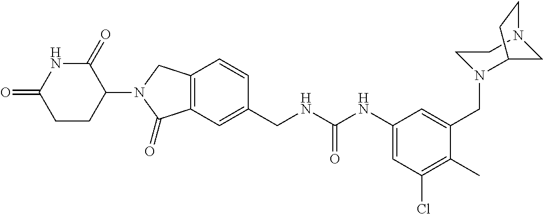 [SMILES: Cc1c(Cl)cc(NC(=O)NCc2ccc3c(c2)C(=O)N(C2CCC(=O)NC2=O)C3)cc1CN1CCN2CCC1C2] | 154 |
| 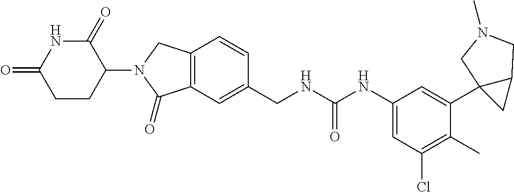 [SMILES: Cc1c(Cl)cc(NC(=O)NCc2ccc3c(c2)C(=O)N(C2CCC(=O)NC2=O)C3)cc1C12CC1CN(C)C2] | 155 |
| 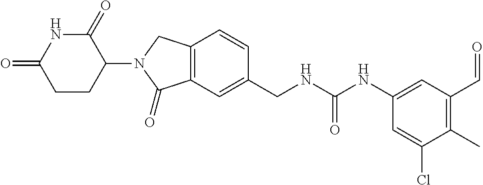 [SMILES: Cc1c(Cl)cc(NC(=O)NCc2ccc3c(c2)C(=O)N(C2CCC(=O)NC2=O)C3)cc1C=O] | 156 |
| 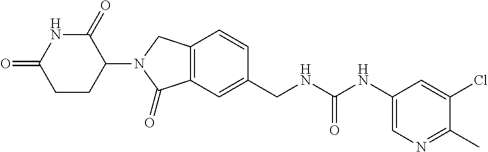 [SMILES: Cc1ncc(NC(=O)NCc2ccc3c(c2)C(=O)N(C2CCC(=O)NC2=O)C3)cc1Cl] | 157 |
| 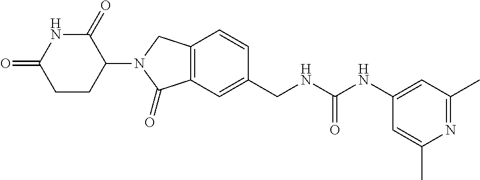 [SMILES: Cc1cc(NC(=O)NCc2ccc3c(c2)C(=O)N(C2CCC(=O)NC2=O)C3)cc(C)n1] | 158 |
| 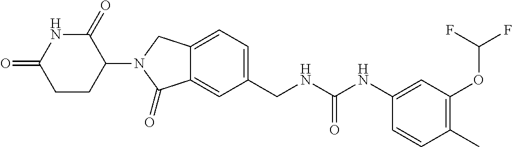 [SMILES: Cc1ccc(NC(=O)NCc2ccc3c(c2)C(=O)N(C2CCC(=O)NC2=O)C3)cc1OC(F)F] | 159 |
| 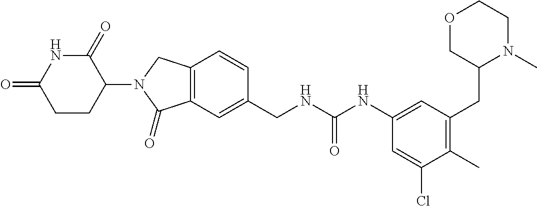 [SMILES: Cc1c(Cl)cc(NC(=O)NCc2ccc3c(c2)C(=O)N(C2CCC(=O)NC2=O)C3)cc1CC1COCCN1C] | 160 |

TABLE 2
Specific examples 200-440

| Compound | No. |
|---|---|
| 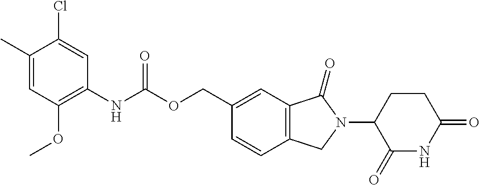 [SMILES: COc1cc(C)c(Cl)cc1NC(=O)OCc1ccc2c(c1)C(=O)N(C1CCC(=O)NC1=O)C2] | 201 |
| 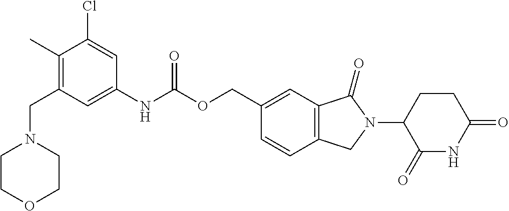 [SMILES: Cc1c(Cl)cc(NC(=O)OCc2ccc3c(c2)C(=O)N(C2CCC(=O)NC2=O)C3)cc1CN1CCOCC1] | 202 |
| 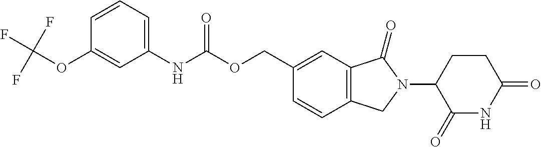 [SMILES: O=C1CCC(N2Cc3ccc(COC(=O)Nc4cccc(OC(F)(F)F)c4)cc3C2=O)C(=O)N1] | 203 |
| 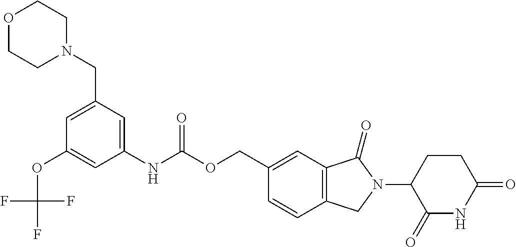 [SMILES: O=C1CCC(N2Cc3ccc(COC(=O)Nc4cc(CN5CCOCC5)cc(OC(F)(F)F)c4)cc3C2=O)C(=O)N1] | 204 |
| 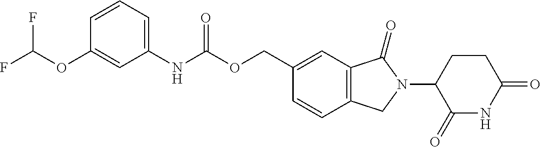 [SMILES: O=C1CCC(N2Cc3ccc(COC(=O)Nc4cccc(OC(F)F)c4)cc3C2=O)C(=O)N1] | 205 |
| 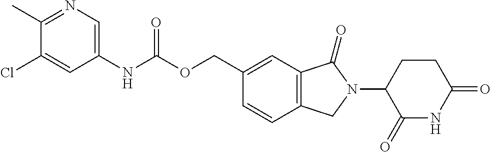 [SMILES: Cc1ncc(NC(=O)OCc2ccc3c(c2)C(=O)N(C2CCC(=O)NC2=O)C3)cc1Cl] | 206 |
| 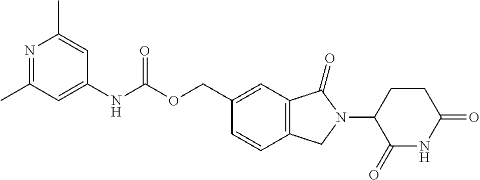 [SMILES: Cc1cc(NC(=O)OCc2ccc3c(c2)C(=O)N(C2CCC(=O)NC2=O)C3)cc(C)n1] | 207 |
| 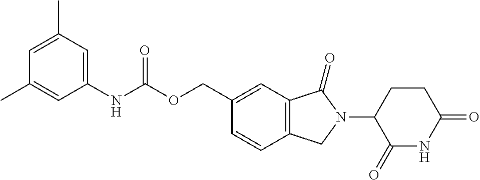 [SMILES: Cc1cc(C)cc(NC(=O)OCc2ccc3c(c2)C(=O)N(C2CCC(=O)NC2=O)C3)c1] | 208 |
| 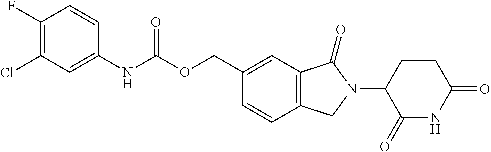 [SMILES: O=C1CCC(N2Cc3ccc(COC(=O)Nc4ccc(F)c(Cl)c4)cc3C2=O)C(=O)N1] | 209 |
| 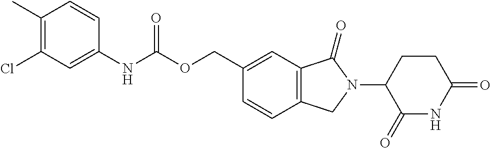 [SMILES: Cc1ccc(NC(=O)OCc2ccc3c(c2)C(=O)N(C2CCC(=O)NC2=O)C3)cc1Cl] | 210 |
| 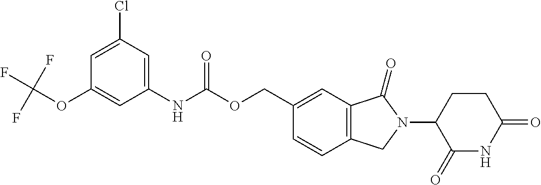 [SMILES: O=C1CCC(N2Cc3ccc(COC(=O)Nc4cc(Cl)cc(OC(F)(F)F)c4)cc3C2=O)C(=O)N1] | 211 |
| 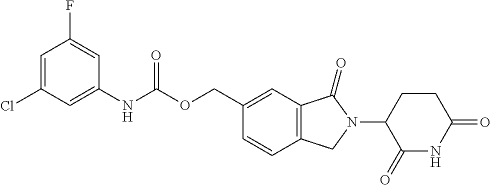 [SMILES: O=C1CCC(N2Cc3ccc(COC(=O)Nc4cc(F)cc(Cl)c4)cc3C2=O)C(=O)N1] | 212 |
| 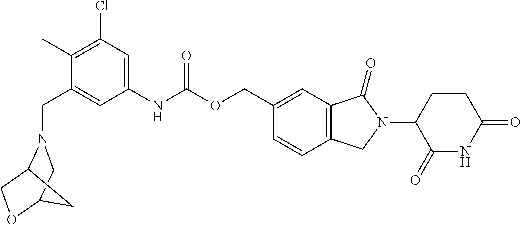 [SMILES: Cc1c(Cl)cc(NC(=O)OCc2ccc3c(c2)C(=O)N(C2CCC(=O)NC2=O)C3)cc1CN1CC2CC1CO2] | 213 |
| 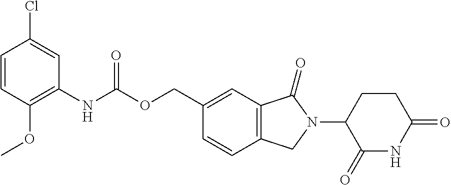 [SMILES: COc1ccc(Cl)cc1NC(=O)OCc1ccc2c(c1)C(=O)N(C1CCC(=O)NC1=O)C2] | 214 |
| 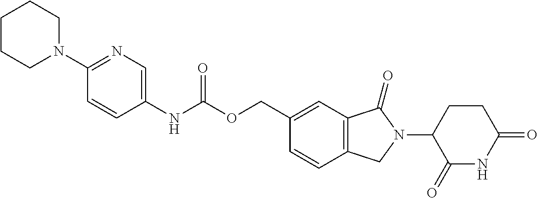 [SMILES: O=C1CCC(N2Cc3ccc(COC(=O)Nc4ccc(N5CCCCC5)nc4)cc3C2=O)C(=O)N1] | 215 |
| 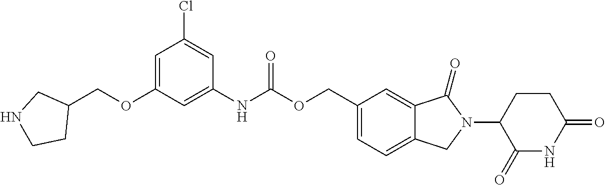 [SMILES: O=C1CCC(N2Cc3ccc(COC(=O)Nc4cc(Cl)cc(OCC5CCNC5)c4)cc3C2=O)C(=O)N1] | 216 |
| 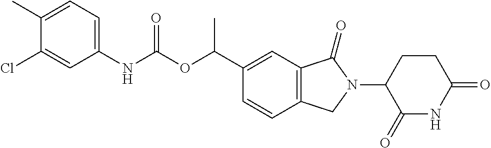 [SMILES: Cc1ccc(NC(=O)OC(C)c2ccc3c(c2)C(=O)N(C2CCC(=O)NC2=O)C3)cc1Cl] | 217 |
| 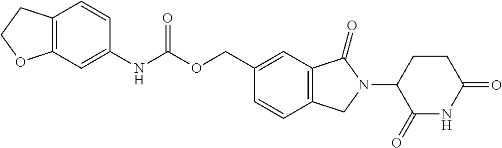 [SMILES: O=C1CCC(N2Cc3ccc(COC(=O)Nc4ccc5c(c4)OCC5)cc3C2=O)C(=O)N1] | 218 |
| 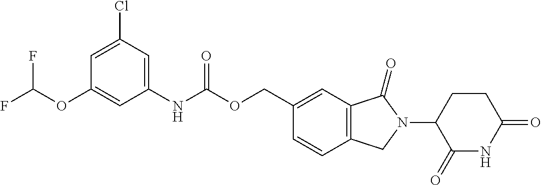 [SMILES: O=C1CCC(N2Cc3ccc(COC(=O)Nc4cc(Cl)cc(OC(F)F)c4)cc3C2=O)C(=O)N1] | 219 |
| 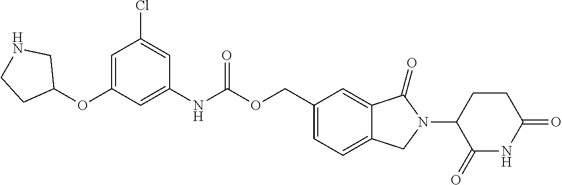 [SMILES: O=C1CCC(N2Cc3ccc(COC(=O)Nc4cc(Cl)cc(OC5CCNC5)c4)cc3C2=O)C(=O)N1] | 220 |
| 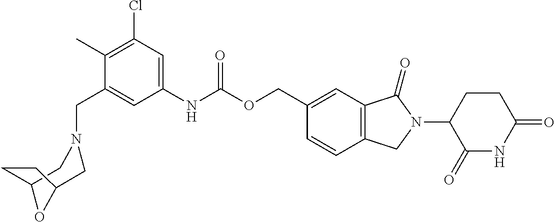 [SMILES: Cc1c(Cl)cc(NC(=O)OCc2ccc3c(c2)C(=O)N(C2CCC(=O)NC2=O)C3)cc1CN1CC2CCC(C1)O2] | 221 |
| 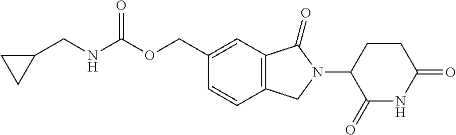 [SMILES: O=C1CCC(N2Cc3ccc(COC(=O)NCC4CC4)cc3C2=O)C(=O)N1] | 222 |
| 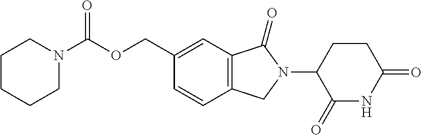 [SMILES: O=C1CCC(N2Cc3ccc(COC(=O)N4CCCCC4)cc3C2=O)C(=O)N1] | 223 |
| 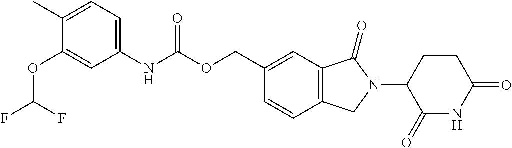 [SMILES: Cc1ccc(NC(=O)OCc2ccc3c(c2)C(=O)N(C2CCC(=O)NC2=O)C3)cc1OC(F)F] | 224 |
| 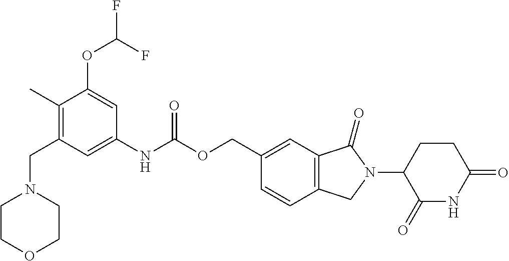 [SMILES: Cc1c(CN2CCOCC2)cc(NC(=O)OCc2ccc3c(c2)C(=O)N(C2CCC(=O)NC2=O)C3)cc1OC(F)F] | 225 |
| 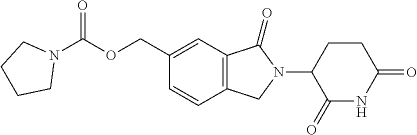 [SMILES: O=C1CCC(N2Cc3ccc(COC(=O)N4CCCC4)cc3C2=O)C(=O)N1] | 226 |
| 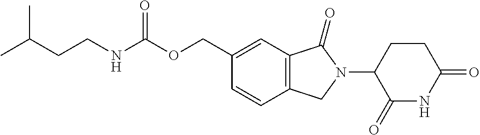 [SMILES: CC(C)CCNC(=O)OCc1ccc2c(c1)C(=O)N(C1CCC(=O)NC1=O)C2] | 227 |
| 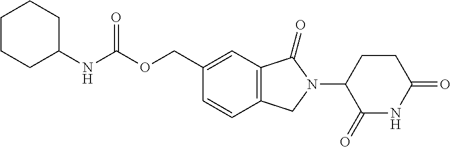 [SMILES: O=C1CCC(N2Cc3ccc(COC(=O)NC4CCCCC4)cc3C2=O)C(=O)N1] | 28 |
| 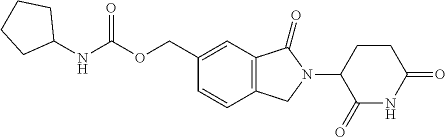 [SMILES: O=C1CCC(N2Cc3ccc(COC(=O)NC4CCCC4)cc3C2=O)C(=O)N1] | 229 |
| 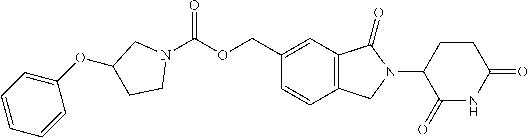 [SMILES: O=C1CCC(N2Cc3ccc(COC(=O)N4CCC(Oc5ccccc5)C4)cc3C2=O)C(=O)N1] | 230 |
| 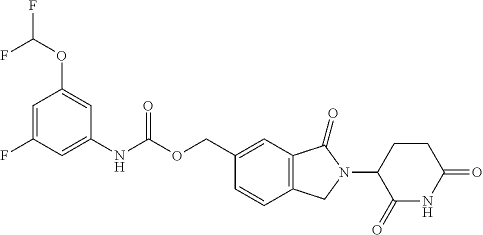 [SMILES: O=C1CCC(N2Cc3ccc(COC(=O)Nc4cc(F)cc(OC(F)F)c4)cc3C2=O)C(=O)N1] | 231 |
| 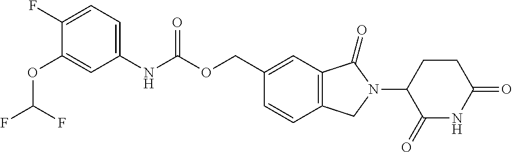 [SMILES: O=C1CCC(N2Cc3ccc(COC(=O)Nc4ccc(F)c(OC(F)F)c4)cc3C2=O)C(=O)N1] | 232 |
| 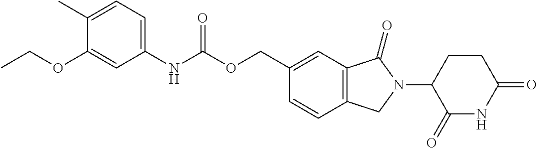 [SMILES: CCOc1cc(NC(=O)OCc2ccc3c(c2)C(=O)N(C2CCC(=O)NC2=O)C3)ccc1C] | 233 |
| 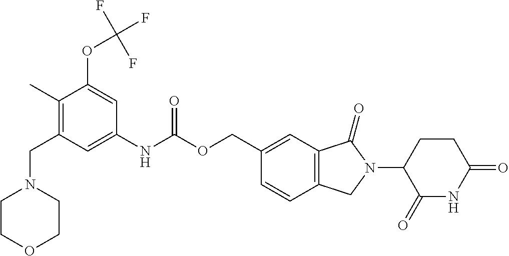 [SMILES: Cc1c(CN2CCOCC2)cc(NC(=O)OCc2ccc3c(c2)C(=O)N(C2CCC(=O)NC2=O)C3)cc1OC(F)(F)F] | 234 |
| 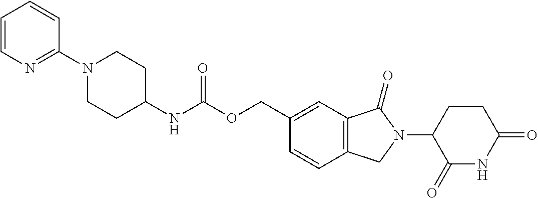 [SMILES: O=C1CCC(N2Cc3ccc(COC(=O)NC4CCN(c5ccccn5)CC4)cc3C2=O)C(=O)N1] | 235 |
| 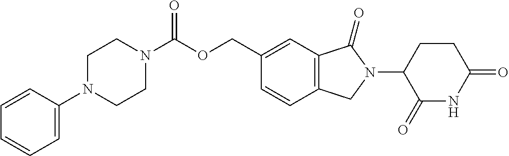 [SMILES: O=C1CCC(N2Cc3ccc(COC(=O)N4CCN(c5ccccc5)CC4)cc3C2=O)C(=O)N1] | 236 |
| 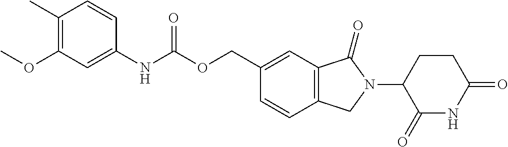 [SMILES: COc1cc(NC(=O)OCc2ccc3c(c2)C(=O)N(C2CCC(=O)NC2=O)C3)ccc1C] | 237 |
| 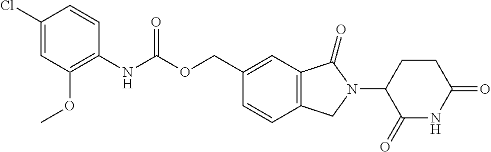 [SMILES: COc1cc(Cl)ccc1NC(=O)OCc1ccc2c(c1)C(=O)N(C1CCC(=O)NC1=O)C2] | 238 |
| 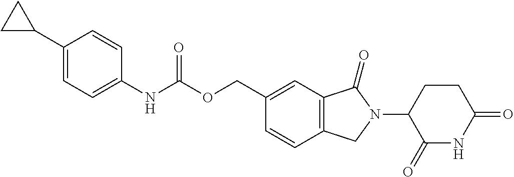 [SMILES: O=C1CCC(N2Cc3ccc(COC(=O)Nc4ccc(C5CC5)cc4)cc3C2=O)C(=O)N1] | 239 |
| 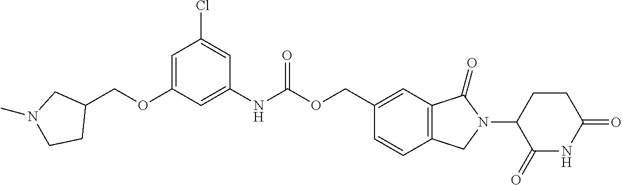 [SMILES: CN1CCC(COc2cc(Cl)cc(NC(=O)OCc3ccc4c(c3)C(=O)N(C3CCC(=O)NC3=O)C4)c2)C1] | 240 |
| 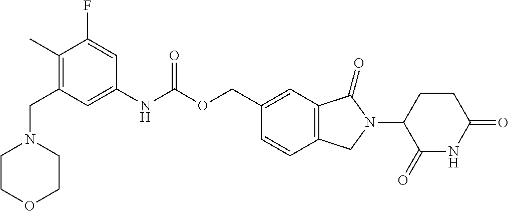 [SMILES: Cc1c(F)cc(NC(=O)OCc2ccc3c(c2)C(=O)N(C2CCC(=O)NC2=O)C3)cc1CN1CCOCC1] | 241 |
| 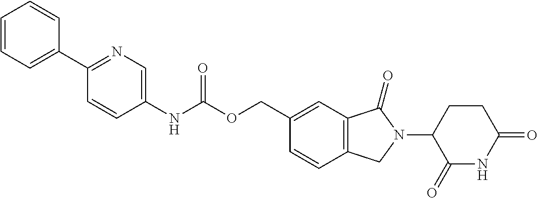 [SMILES: O=C1CCC(N2Cc3ccc(COC(=O)Nc4ccc(-c5ccccc5)nc4)cc3C2=O)C(=O)N1] | 242 |
| 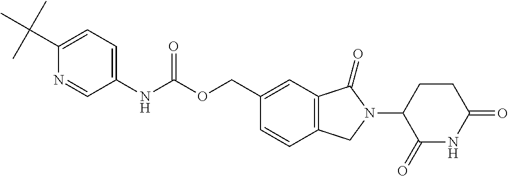 [SMILES: CC(C)(C)c1ccc(NC(=O)OCc2ccc3c(c2)C(=O)N(C2CCC(=O)NC2=O)C3)cn1] | 243 |
| 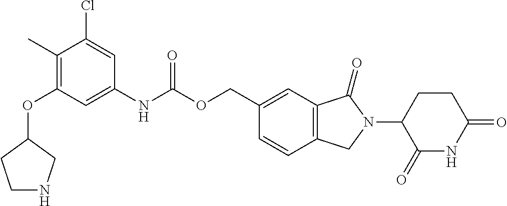 [SMILES: Cc1c(Cl)cc(NC(=O)OCc2ccc3c(c2)C(=O)N(C2CCC(=O)NC2=O)C3)cc1OC1CCNC1] | 244 |
| 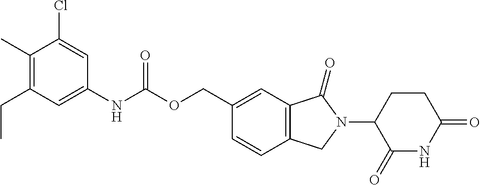 [SMILES: CCc1cc(NC(=O)OCc2ccc3c(c2)C(=O)N(C2CCC(=O)NC2=O)C3)cc(Cl)c1C] | 245 |
| 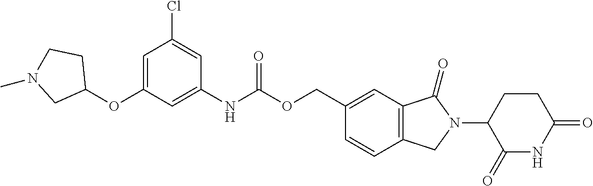 [SMILES: CN1CCC(Oc2cc(Cl)cc(NC(=O)OCc3ccc4c(c3)C(=O)N(C3CCC(=O)NC3=O)C4)c2)C1] | 246 |
| 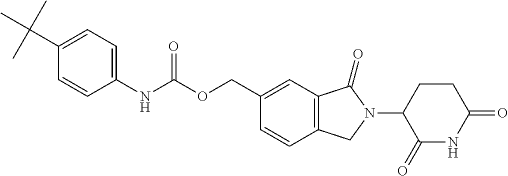 [SMILES: CC(C)(C)c1ccc(NC(=O)OCc2ccc3c(c2)C(=O)N(C2CCC(=O)NC2=O)C3)cc1] | 247 |
| 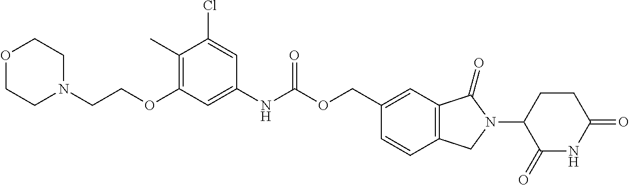 [SMILES: Cc1c(Cl)cc(NC(=O)OCc2ccc3c(c2)C(=O)N(C2CCC(=O)NC2=O)C3)cc1OCCN1CCOCC1] | 248 |
| 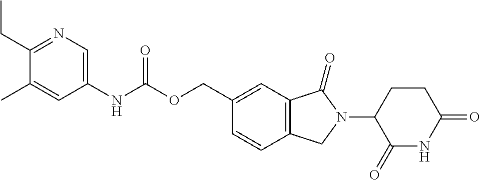 [SMILES: CCc1ncc(NC(=O)OCc2ccc3c(c2)C(=O)N(C2CCC(=O)NC2=O)C3)cc1C] | 249 |
| 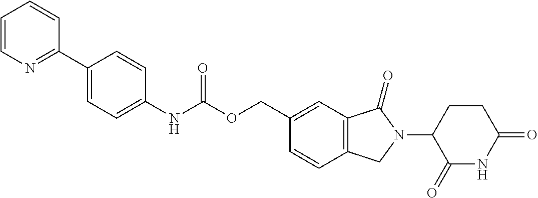 [SMILES: O=C1CCC(N2Cc3ccc(COC(=O)Nc4ccc(-c5ccccn5)cc4)cc3C2=O)C(=O)N1] | 250 |
| 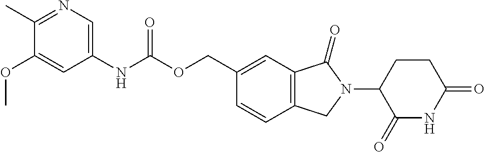 [SMILES: COc1cc(NC(=O)OCc2ccc3c(c2)C(=O)N(C2CCC(=O)NC2=O)C3)cnc1C] | 251 |
| 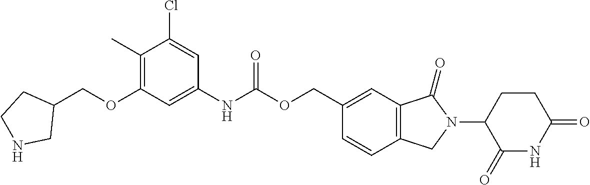 [SMILES: Cc1c(Cl)cc(NC(=O)OCc2ccc3c(c2)C(=O)N(C2CCC(=O)NC2=O)C3)cc1OCC1CCNC1] | 252 |
| 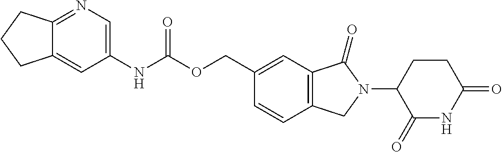 [SMILES: O=C1CCC(N2Cc3ccc(COC(=O)Nc4cnc5c(c4)CCC5)cc3C2=O)C(=O)N1] | 253 |
| 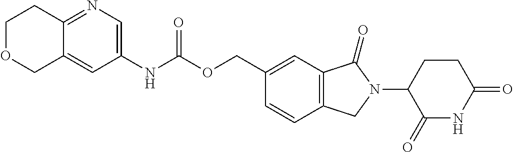 [SMILES: O=C1CCC(N2Cc3ccc(COC(=O)Nc4cnc5c(c4)COCC5)cc3C2=O)C(=O)N1] | 254 |
| 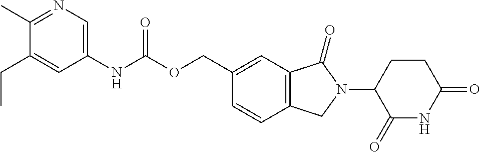 [SMILES: CCc1cc(NC(=O)OCc2ccc3c(c2)C(=O)N(C2CCC(=O)NC2=O)C3)cnc1C] | 255 |
| 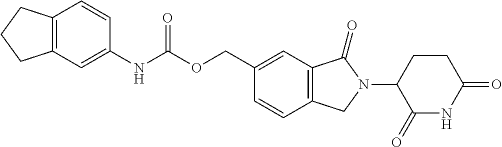 [SMILES: O=C1CCC(N2Cc3ccc(COC(=O)Nc4ccc5c(c4)CCC5)cc3C2=O)C(=O)N1] | 256 |
| 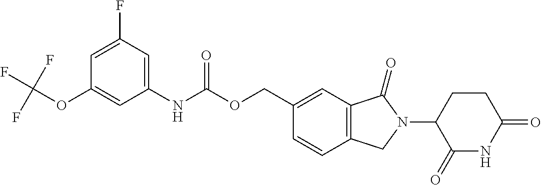 [SMILES: O=C1CCC(N2Cc3ccc(COC(=O)Nc4cc(F)cc(OC(F)(F)F)c4)cc3C2=O)C(=O)N1] | 257 |
| 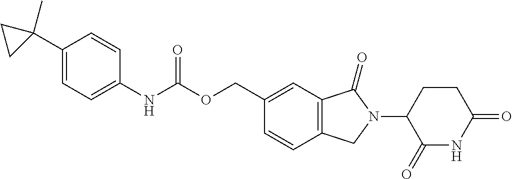 [SMILES: CC1(c2ccc(NC(=O)OCc3ccc4c(c3)C(=O)N(C3CCC(=O)NC3=O)C4)cc2)CC1] | 258 |
| 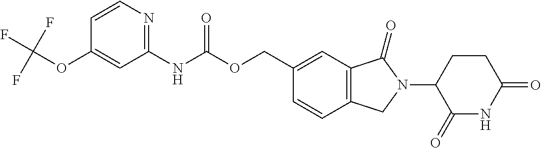 [SMILES: O=C1CCC(N2Cc3ccc(COC(=O)Nc4cc(OC(F)(F)F)ccn4)cc3C2=O)C(=O)N1] | 259 |
| 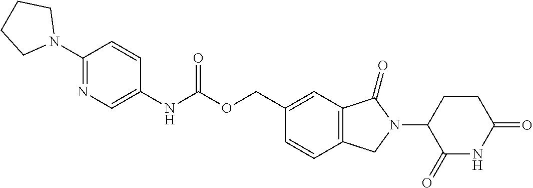 [SMILES: O=C1CCC(N2Cc3ccc(COC(=O)Nc4ccc(N5CCCC5)nc4)cc3C2=O)C(=O)N1] | 260 |
| 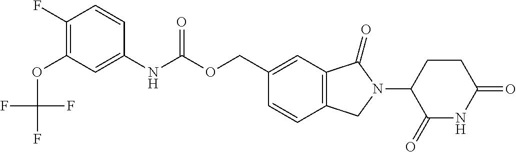 [SMILES: O=C1CCC(N2Cc3ccc(COC(=O)Nc4ccc(F)c(OC(F)(F)F)c4)cc3C2=O)C(=O)N1] | 261 |
| 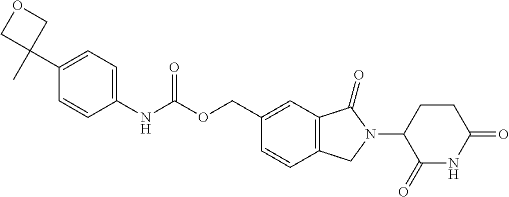 [SMILES: CC1(c2ccc(NC(=O)OCc3ccc4c(c3)C(=O)N(C3CCC(=O)NC3=O)C4)cc2)COC1] | 262 |
| 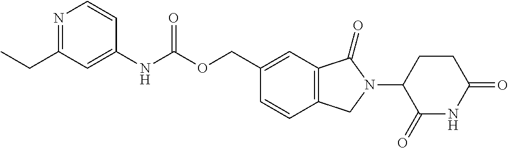 [SMILES: CCc1cc(NC(=O)OCc2ccc3c(c2)C(=O)N(C2CCC(=O)NC2=O)C3)ccn1] | 263 |
| 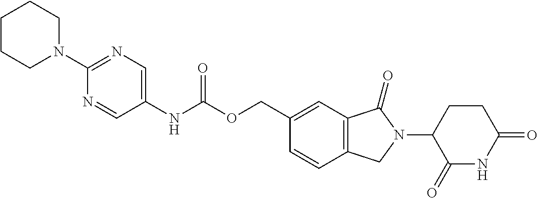 [SMILES: O=C1CCC(N2Cc3ccc(COC(=O)Nc4cnc(N5CCCCC5)nc4)cc3C2=O)C(=O)N1] | 264 |
| 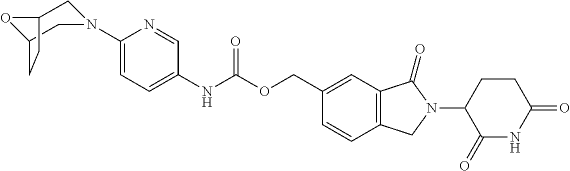 [SMILES: O=C1CCC(N2Cc3ccc(COC(=O)Nc4ccc(N5CC6CCC(C5)O6)nc4)cc3C2=O)C(=O)N1] | 265 |
| 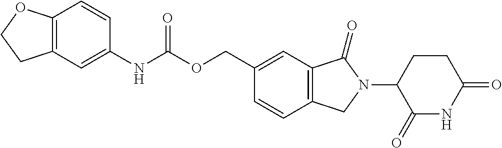 [SMILES: O=C1CCC(N2Cc3ccc(COC(=O)Nc4ccc5c(c4)CCO5)cc3C2=O)C(=O)N1] | 266 |
| 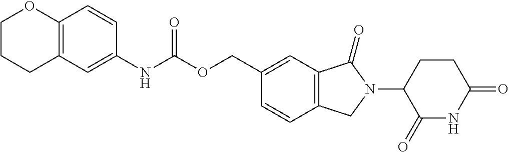 [SMILES: O=C1CCC(N2Cc3ccc(COC(=O)Nc4ccc5c(c4)CCCO5)cc3C2=O)C(=O)N1] | 267 |
| 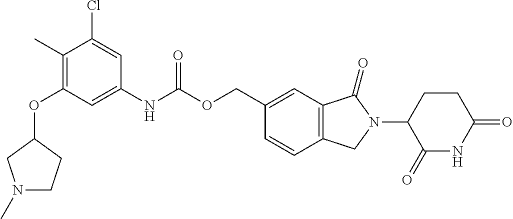 [SMILES: Cc1c(Cl)cc(NC(=O)OCc2ccc3c(c2)C(=O)N(C2CCC(=O)NC2=O)C3)cc1OC1CCN(C)C1] | 268 |
| 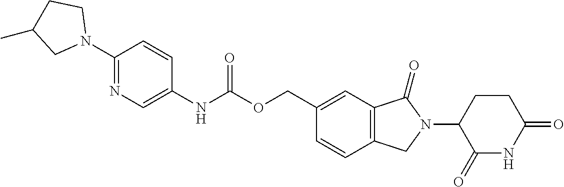 [SMILES: CC1CCN(c2ccc(NC(=O)OCc3ccc4c(c3)C(=O)N(C3CCC(=O)NC3=O)C4)cn2)C1] | 269 |
| 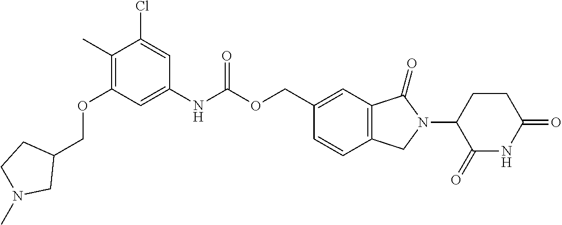 [SMILES: Cc1c(Cl)cc(NC(=O)OCc2ccc3c(c2)C(=O)N(C2CCC(=O)NC2=O)C3)cc1OCC1CCN(C)C1] | 270 |
| 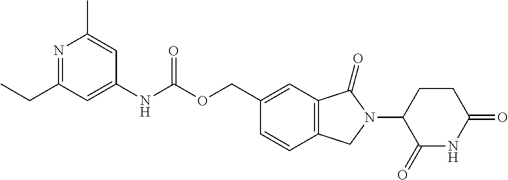 [SMILES: CCc1cc(NC(=O)OCc2ccc3c(c2)C(=O)N(C2CCC(=O)NC2=O)C3)cc(C)n1] | 271 |
| 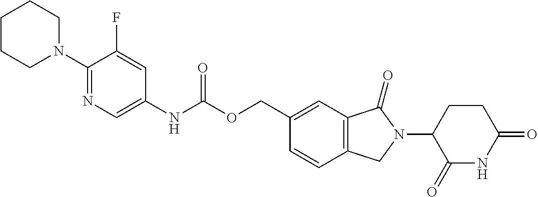 [SMILES: O=C1CCC(N2Cc3ccc(COC(=O)Nc4cnc(N5CCCCC5)c(F)c4)cc3C2=O)C(=O)N1] | 272 |
| 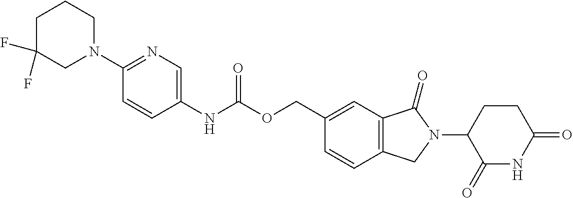 [SMILES: O=C1CCC(N2Cc3ccc(COC(=O)Nc4ccc(N5CCCC(F)(F)C5)nc4)cc3C2=O)C(=O)N1] | 273 |
| 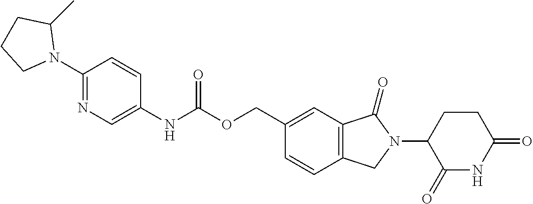 [SMILES: CC1CCCN1c1ccc(NC(=O)OCc2ccc3c(c2)C(=O)N(C2CCC(=O)NC2=O)C3)cn1] | 274 |
| 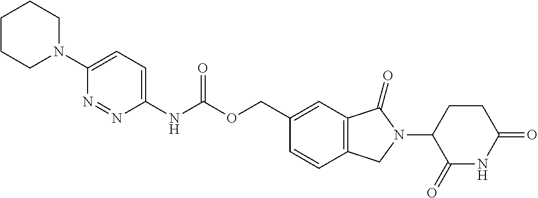 [SMILES: O=C1CCC(N2Cc3ccc(COC(=O)Nc4ccc(N5CCCCC5)nn4)cc3C2=O)C(=O)N1] | 275 |
| 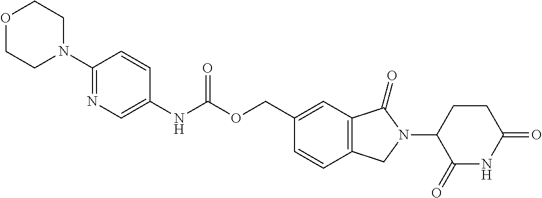 [SMILES: O=C1CCC(N2Cc3ccc(COC(=O)Nc4ccc(N5CCOCC5)nc4)cc3C2=O)C(=O)N1] | 276 |
| 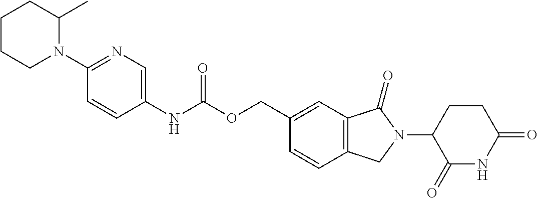 [SMILES: CC1CCCCN1c1ccc(NC(=O)OCc2ccc3c(c2)C(=O)N(C2CCC(=O)NC2=O)C3)cn1] | 277 |
| 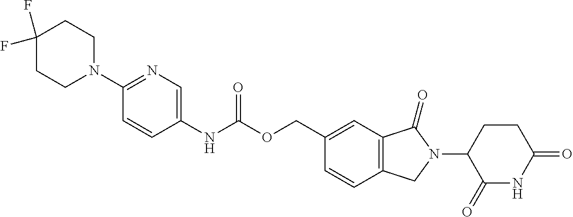 [SMILES: O=C1CCC(N2Cc3ccc(COC(=O)Nc4ccc(N5CCC(F)(F)CC5)nc4)cc3C2=O)C(=O)N1] | 278 |
| 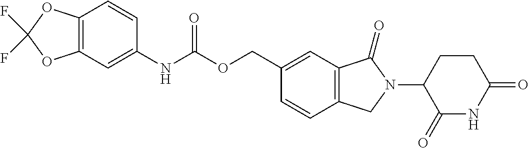 [SMILES: O=C1CCC(N2Cc3ccc(COC(=O)Nc4ccc5c(c4)OC(F)(F)O5)cc3C2=O)C(=O)N1] | 279 |
| 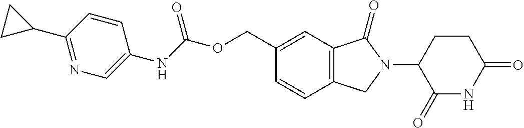 [SMILES: O=C1CCC(N2Cc3ccc(COC(=O)Nc4ccc(C5CC5)nc4)cc3C2=O)C(=O)N1] | 280 |
| 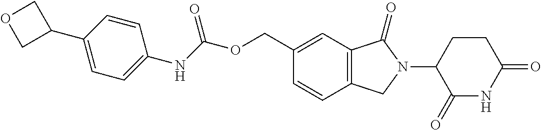 [SMILES: O=C1CCC(N2Cc3ccc(COC(=O)Nc4ccc(C5COC5)cc4)cc3C2=O)C(=O)N1] | 281 |
| 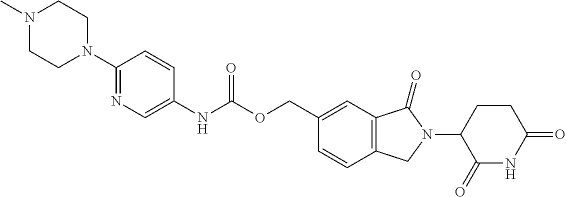 [SMILES: CN1CCN(c2ccc(NC(=O)OCc3ccc4c(c3)C(=O)N(C3CCC(=O)NC3=O)C4)cn2)CC1] | 282 |
| 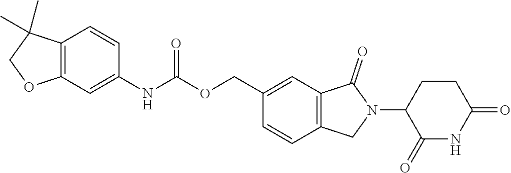 [SMILES: CC1(C)COc2cc(NC(=O)OCc3ccc4c(c3)C(=O)N(C3CCC(=O)NC3=O)C4)ccc21] | 283 |
| 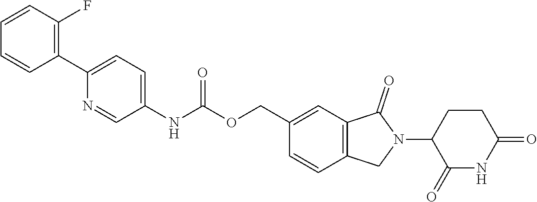 [SMILES: O=C1CCC(N2Cc3ccc(COC(=O)Nc4ccc(-c5ccccc5F)nc4)cc3C2=O)C(=O)N1] | 284 |
| 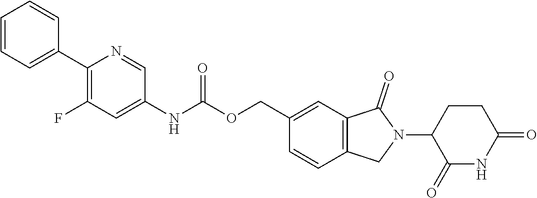 [SMILES: O=C1CCC(N2Cc3ccc(COC(=O)Nc4cnc(-c5ccccc5)c(F)c4)cc3C2=O)C(=O)N1] | 285 |
| 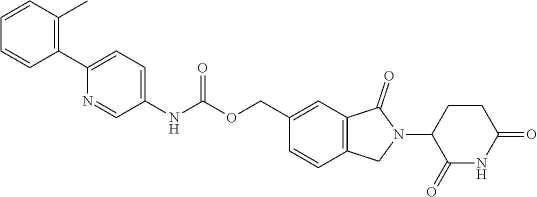 [SMILES: Cc1ccccc1-c1ccc(NC(=O)OCc2ccc3c(c2)C(=O)N(C2CCC(=O)NC2=O)C3)cn1] | 286 |
| 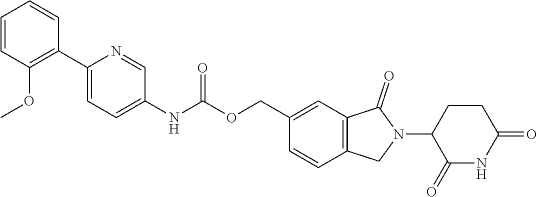 [SMILES: COc1ccccc1-c1ccc(NC(=O)OCc2ccc3c(c2)C(=O)N(C2CCC(=O)NC2=O)C3)cn1] | 287 |
| 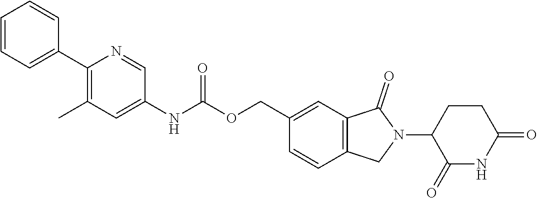 [SMILES: Cc1cc(NC(=O)OCc2ccc3c(c2)C(=O)N(C2CCC(=O)NC2=O)C3)cnc1-c1ccccc1] | 288 |
| 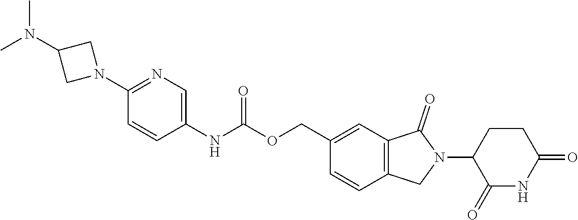 [SMILES: CN(C)C1CN(c2ccc(NC(=O)OCc3ccc4c(c3)C(=O)N(C3CCC(=O)NC3=O)C4)cn2)C1] | 289 |
| 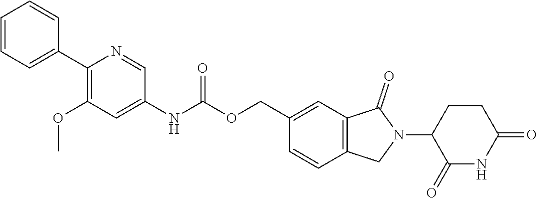 [SMILES: COc1cc(NC(=O)OCc2ccc3c(c2)C(=O)N(C2CCC(=O)NC2=O)C3)cnc1-c1ccccc1] | 290 |
| 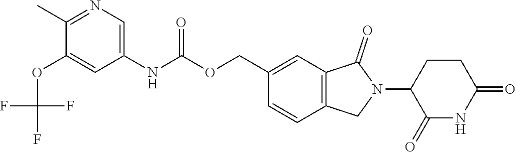 [SMILES: Cc1ncc(NC(=O)OCc2ccc3c(c2)C(=O)N(C2CCC(=O)NC2=O)C3)cc1OC(F)(F)F] | 291 |
| 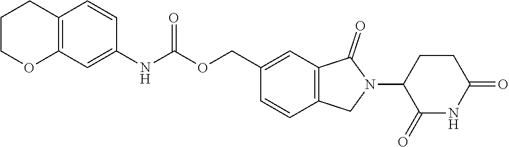 [SMILES: O=C1CCC(N2Cc3ccc(COC(=O)Nc4ccc5c(c4)OCCC5)cc3C2=O)C(=O)N1] | 292 |
| 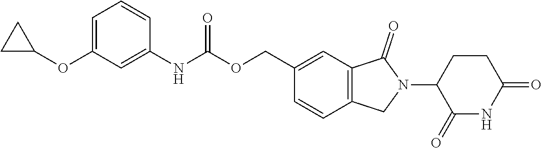 [SMILES: O=C1CCC(N2Cc3ccc(COC(=O)Nc4cccc(OC5CC5)c4)cc3C2=O)C(=O)N1] | 293 |
| 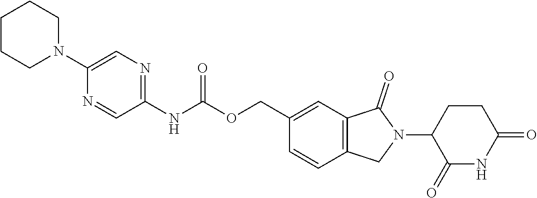 [SMILES: O=C1CCC(N2Cc3ccc(COC(=O)Nc4cnc(N5CCCCC5)cn4)cc3C2=O)C(=O)N1] | 294 |
| 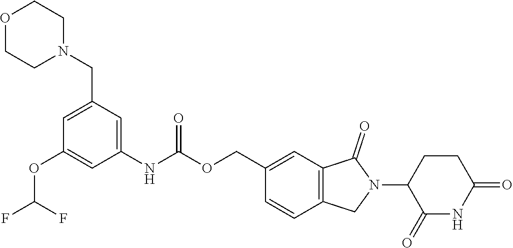 [SMILES: O=C1CCC(N2Cc3ccc(COC(=O)Nc4cc(CN5CCOCC5)cc(OC(F)F)c4)cc3C2=O)C(=O)N1] | 295 |
| 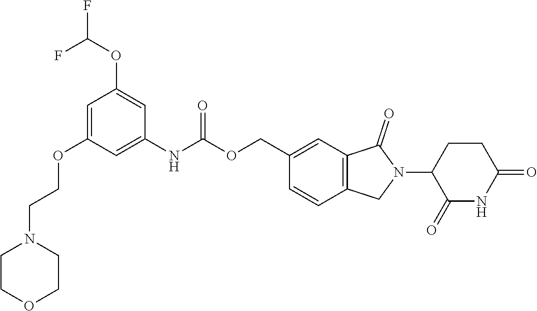 [SMILES: O=C1CCC(N2Cc3ccc(COC(=O)Nc4cc(OCCN5CCOCC5)cc(OC(F)F)c4)cc3C2=O)C(=O)N1] | 296 |
| 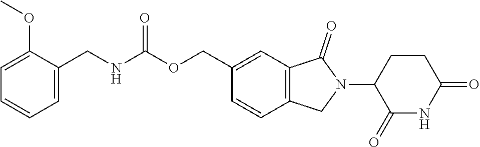 [SMILES: COc1ccccc1CNC(=O)OCc1ccc2c(c1)C(=O)N(C1CCC(=O)NC1=O)C2] | 297 |
| 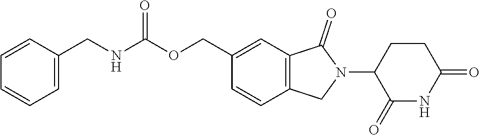 [SMILES: O=C1CCC(N2Cc3ccc(COC(=O)NCc4ccccc4)cc3C2=O)C(=O)N1] | 298 |
| 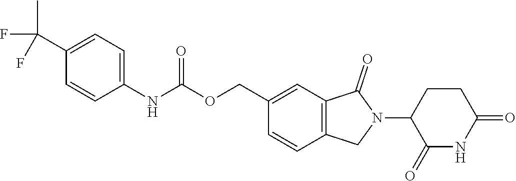 [SMILES: CC(F)(F)c1ccc(NC(=O)OCc2ccc3c(c2)C(=O)N(C2CCC(=O)NC2=O)C3)cc1] | 299 |
| 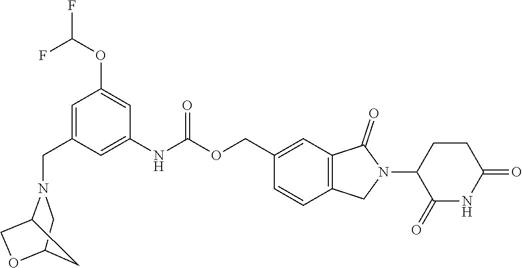 [SMILES: O=C1CCC(N2Cc3ccc(COC(=O)Nc4cc(CN5CC6CC5CO6)cc(OC(F)F)c4)cc3C2=O)C(=O)N1] | 300 |
| 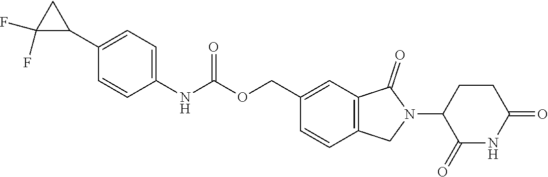 [SMILES: O=C1CCC(N2Cc3ccc(COC(=O)Nc4ccc(C5CC5(F)F)cc4)cc3C2=O)C(=O)N1] | 301 |
| 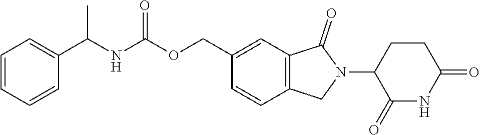 [SMILES: CC(NC(=O)OCc1ccc2c(c1)C(=O)N(C1CCC(=O)NC1=O)C2)c1ccccc1] | 302 |
| 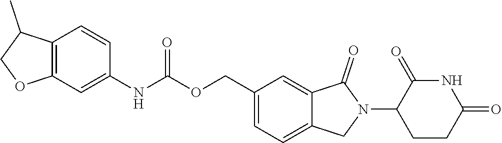 [SMILES: CC1COc2cc(NC(=O)OCc3ccc4c(c3)C(=O)N(C3CCC(=O)NC3=O)C4)ccc21] | 303 |
| 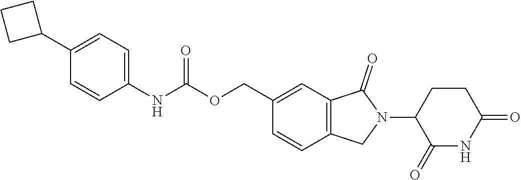 [SMILES: O=C1CCC(N2Cc3ccc(COC(=O)Nc4ccc(C5CCC5)cc4)cc3C2=O)C(=O)N1] | 304 |
| 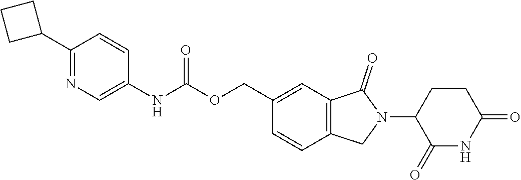 [SMILES: O=C1CCC(N2Cc3ccc(COC(=O)Nc4ccc(C5CCC5)nc4)cc3C2=O)C(=O)N1] | 305 |
| 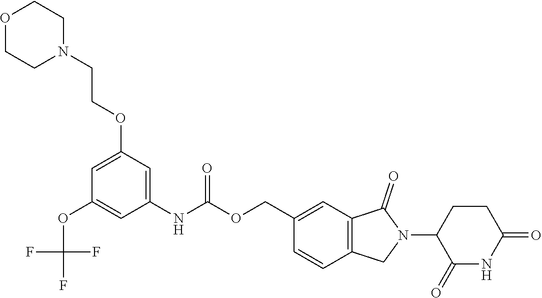 [SMILES: O=C1CCC(N2Cc3ccc(COC(=O)Nc4cc(OCCN5CCOCC5)cc(OC(F)(F)F)c4)cc3C2=O)C(=O)N1] | 306 |
| 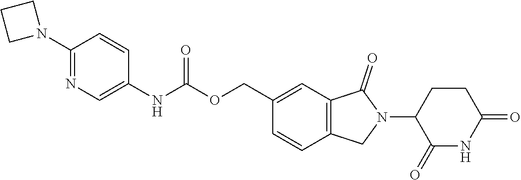 [SMILES: O=C1CCC(N2Cc3ccc(COC(=O)Nc4ccc(N5CCC5)nc4)cc3C2=O)C(=O)N1] | 307 |
| 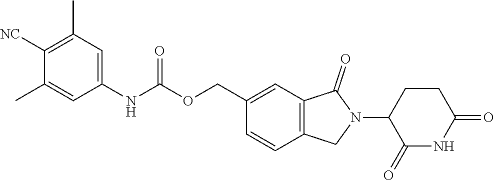 [SMILES: Cc1cc(NC(=O)OCc2ccc3c(c2)C(=O)N(C2CCC(=O)NC2=O)C3)cc(C)c1C#N] | 308 |
| 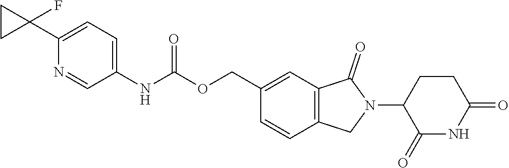 [SMILES: O=C1CCC(N2Cc3ccc(COC(=O)Nc4ccc(C5(F)CC5)nc4)cc3C2=O)C(=O)N1] | 309 |
| 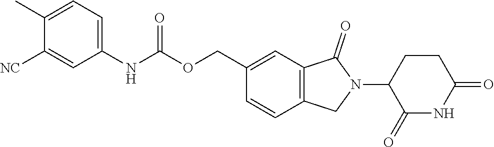 [SMILES: Cc1ccc(NC(=O)OCc2ccc3c(c2)C(=O)N(C2CCC(=O)NC2=O)C3)cc1C#N] | 310 |
| 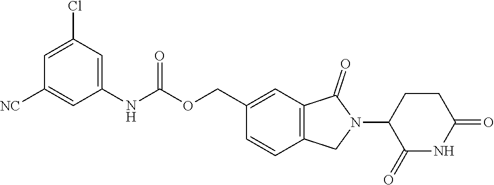 [SMILES: N#Cc1cc(Cl)cc(NC(=O)OCc2ccc3c(c2)C(=O)N(C2CCC(=O)NC2=O)C3)c1] | 311 |
| 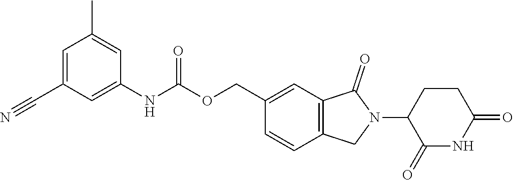 [SMILES: Cc1cc(C#N)cc(NC(=O)OCc2ccc3c(c2)C(=O)N(C2CCC(=O)NC2=O)C3)c1] | 312 |
| 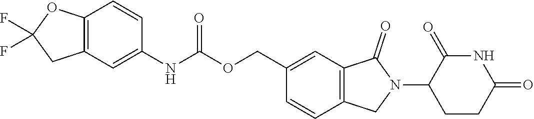 [SMILES: O=C1CCC(N2Cc3ccc(COC(=O)Nc4ccc5c(c4)CC(F)(F)O5)cc3C2=O)C(=O)N1] | 313 |
| 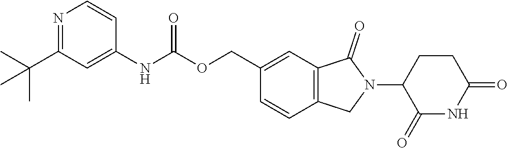 [SMILES: CC(C)(C)c1cc(NC(=O)OCc2ccc3c(c2)C(=O)N(C2CCC(=O)NC2=O)C3)ccn1] | 314 |
| 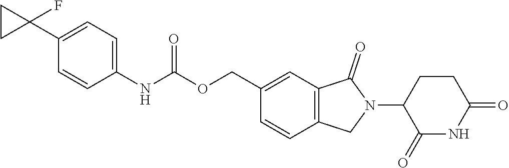 [SMILES: O=C1CCC(N2Cc3ccc(COC(=O)Nc4ccc(C5(F)CC5)cc4)cc3C2=O)C(=O)N1] | 315 |
| 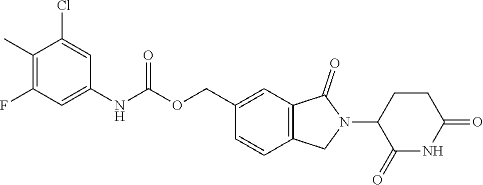 [SMILES: Cc1c(F)cc(NC(=O)OCc2ccc3c(c2)C(=O)N(C2CCC(=O)NC2=O)C3)cc1Cl] | 316 |
| 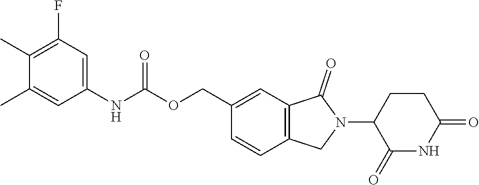 [SMILES: Cc1cc(NC(=O)OCc2ccc3c(c2)C(=O)N(C2CCC(=O)NC2=O)C3)cc(F)c1C] | 317 |
| 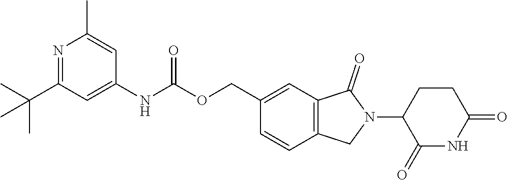 [SMILES: Cc1cc(NC(=O)OCc2ccc3c(c2)C(=O)N(C2CCC(=O)NC2=O)C3)cc(C(C)(C)C)n1] | 318 |
| 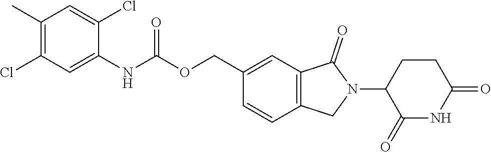 [SMILES: Cc1cc(Cl)c(NC(=O)OCc2ccc3c(c2)C(=O)N(C2CCC(=O)NC2=O)C3)cc1Cl] | 319 |
| 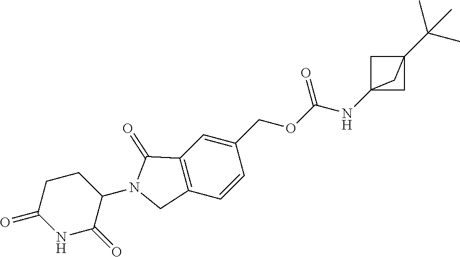 [SMILES: CC(C)(C)C12CC(NC(=O)OCc3ccc4c(c3)C(=O)N(C3CCC(=O)NC3=O)C4)(C1)C2] | 320 |
| 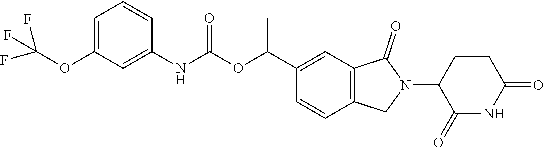 [SMILES: CC(OC(=O)Nc1cccc(OC(F)(F)F)c1)c1ccc2c(c1)C(=O)N(C1CCC(=O)NC1=O)C2] | 321 |
| 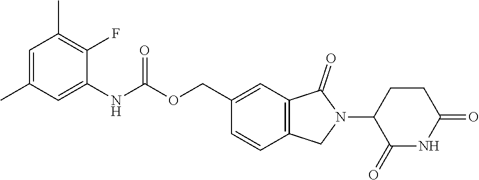 [SMILES: Cc1cc(C)c(F)c(NC(=O)OCc2ccc3c(c2)C(=O)N(C2CCC(=O)NC2=O)C3)c1] | 322 |
| 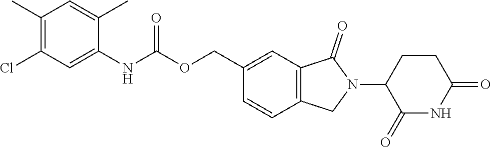 [SMILES: Cc1cc(C)c(NC(=O)OCc2ccc3c(c2)C(=O)N(C2CCC(=O)NC2=O)C3)cc1Cl] | 323 |
| 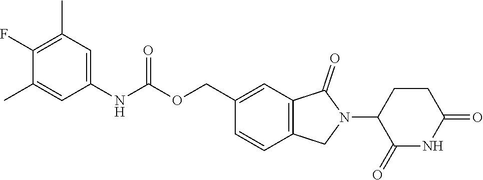 [SMILES: Cc1cc(NC(=O)OCc2ccc3c(c2)C(=O)N(C2CCC(=O)NC2=O)C3)cc(C)c1F] | 324 |
| 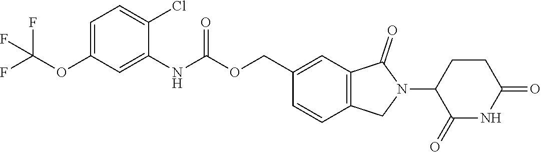 [SMILES: O=C1CCC(N2Cc3ccc(COC(=O)Nc4cc(OC(F)(F)F)ccc4Cl)cc3C2=O)C(=O)N1] | 325 |
| 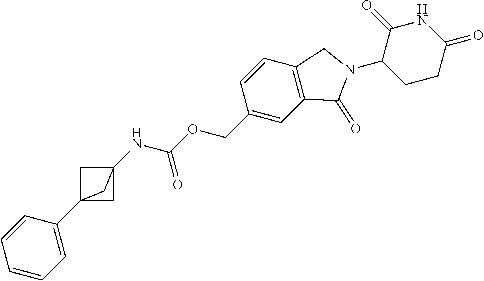 [SMILES: O=C1CCC(N2Cc3ccc(COC(=O)NC45CC(c6ccccc6)(C4)C5)cc3C2=O)C(=O)N1] | 326 |
| 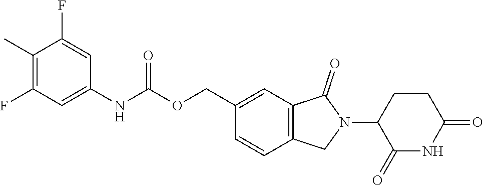 [SMILES: Cc1c(F)cc(NC(=O)OCc2ccc3c(c2)C(=O)N(C2CCC(=O)NC2=O)C3)cc1F] | 327 |
| 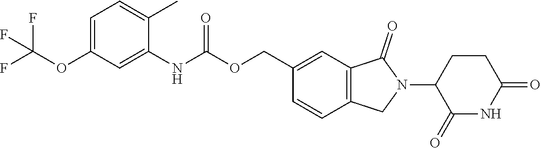 [SMILES: Cc1ccc(OC(F)(F)F)cc1NC(=O)OCc1ccc2c(c1)C(=O)N(C1CCC(=O)NC1=O)C2] | 328 |
| 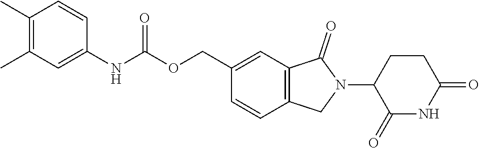 [SMILES: Cc1ccc(NC(=O)OCc2ccc3c(c2)C(=O)N(C2CCC(=O)NC2=O)C3)cc1C] | 329 |
| 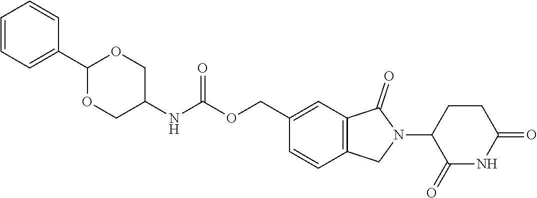 [SMILES: O=C1CCC(N2Cc3ccc(COC(=O)NC4COC(c5ccccc5)OC4)cc3C2=O)C(=O)N1] | 330 |
| 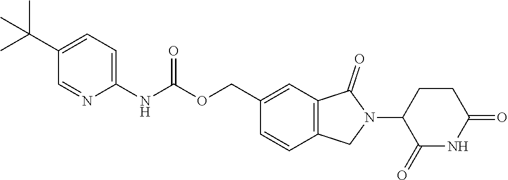 [SMILES: CC(C)(C)c1ccc(NC(=O)OCc2ccc3c(c2)C(=O)N(C2CCC(=O)NC2=O)C3)nc1] | 331 |
| 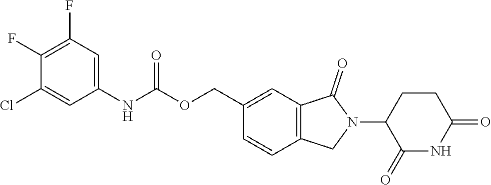 [SMILES: O=C1CCC(N2Cc3ccc(COC(=O)Nc4cc(F)c(F)c(Cl)c4)cc3C2=O)C(=O)N1] | 332 |
| 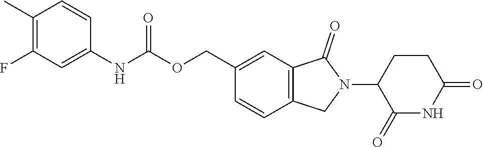 [SMILES: Cc1ccc(NC(=O)OCc2ccc3c(c2)C(=O)N(C2CCC(=O)NC2=O)C3)cc1F] | 333 |
| 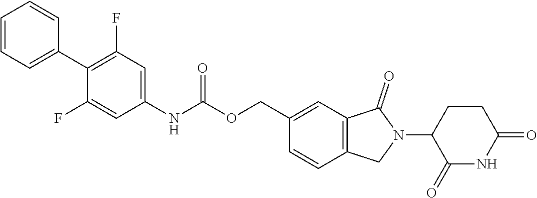 [SMILES: O=C1CCC(N2Cc3ccc(COC(=O)Nc4cc(F)c(-c5ccccc5)c(F)c4)cc3C2=O)C(=O)N1] | 334 |
| 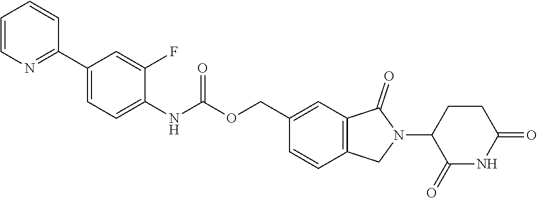 [SMILES: O=C1CCC(N2Cc3ccc(COC(=O)Nc4ccc(-c5ccccn5)cc4F)cc3C2=O)C(=O)N1] | 335 |
| 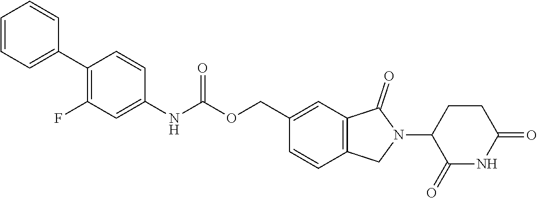 [SMILES: O=C1CCC(N2Cc3ccc(COC(=O)Nc4ccc(-c5ccccc5)c(F)c4)cc3C2=O)C(=O)N1] | 336 |
| 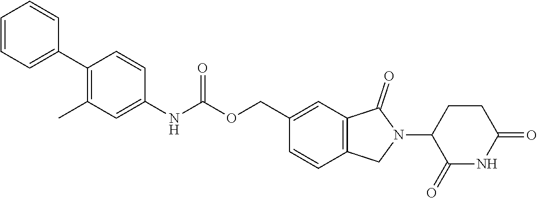 [SMILES: Cc1cc(NC(=O)OCc2ccc3c(c2)C(=O)N(C2CCC(=O)NC2=O)C3)ccc1-c1ccccc1] | 337 |
| 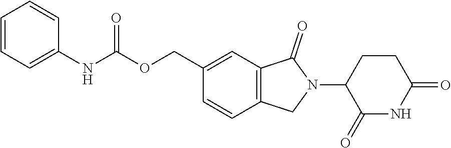 [SMILES: O=C1CCC(N2Cc3ccc(COC(=O)Nc4ccccc4)cc3C2=O)C(=O)N1] | 338 |
| 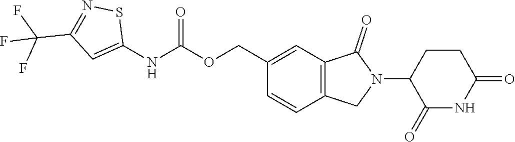 [SMILES: O=C1CCC(N2Cc3ccc(COC(=O)Nc4cc(C(F)(F)F)ns4)cc3C2=O)C(=O)N1] | 339 |
| 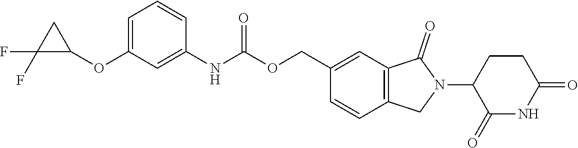 [SMILES: O=C1CCC(N2Cc3ccc(COC(=O)Nc4cccc(OC5CC5(F)F)c4)cc3C2=O)C(=O)N1] | 340 |
| 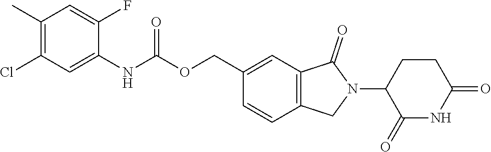 [SMILES: Cc1cc(F)c(NC(=O)OCc2ccc3c(c2)C(=O)N(C2CCC(=O)NC2=O)C3)cc1Cl] | 341 |
| 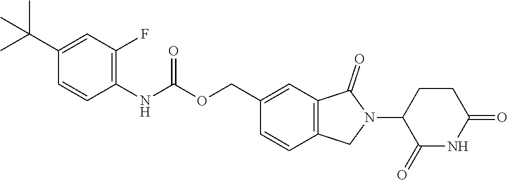 [SMILES: CC(C)(C)c1ccc(NC(=O)OCc2ccc3c(c2)C(=O)N(C2CCC(=O)NC2=O)C3)c(F)c1] | 342 |
| 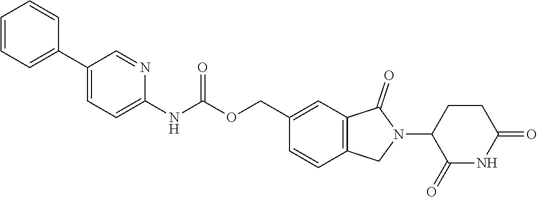 [SMILES: O=C1CCC(N2Cc3ccc(COC(=O)Nc4ccc(-c5ccccc5)cn4)cc3C2=O)C(=O)N1] | 343 |
| 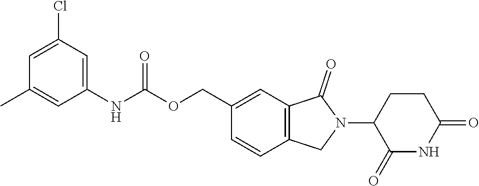 [SMILES: Cc1cc(Cl)cc(NC(=O)OCc2ccc3c(c2)C(=O)N(C2CCC(=O)NC2=O)C3)c1] | 344 |
| 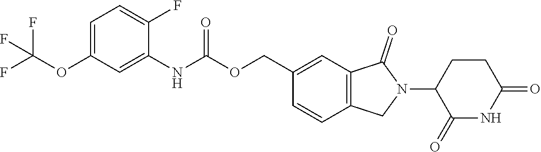 [SMILES: O=C1CCC(N2Cc3ccc(COC(=O)Nc4cc(OC(F)(F)F)ccc4F)cc3C2=O)C(=O)N1] | 345 |
| 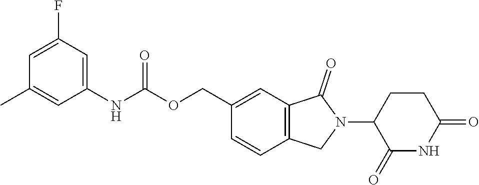 [SMILES: Cc1cc(F)cc(NC(=O)OCc2ccc3c(c2)C(=O)N(C2CCC(=O)NC2=O)C3)c1] | 346 |
| 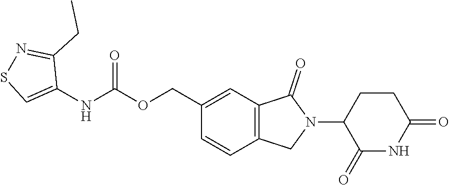 [SMILES: CCc1nscc1NC(=O)OCc1ccc2c(c1)C(=O)N(C1CCC(=O)NC1=O)C2] | 347 |
| 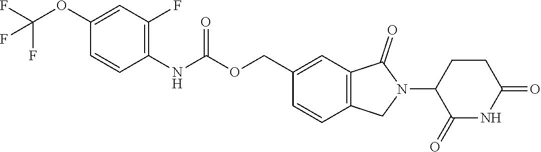 [SMILES: O=C1CCC(N2Cc3ccc(COC(=O)Nc4ccc(OC(F)(F)F)cc4F)cc3C2=O)C(=O)N1] | 348 |
| 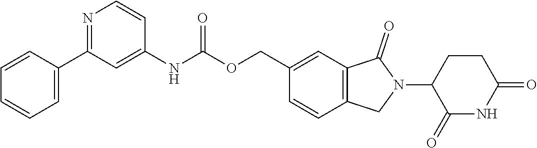 [SMILES: O=C1CCC(N2Cc3ccc(COC(=O)Nc4ccnc(-c5ccccc5)c4)cc3C2=O)C(=O)N1] | 349 |
| 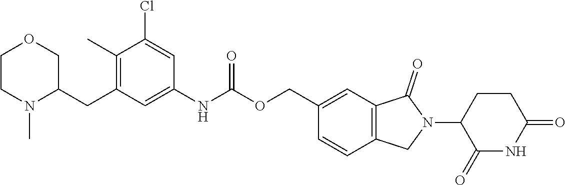 [SMILES: Cc1c(Cl)cc(NC(=O)OCc2ccc3c(c2)C(=O)N(C2CCC(=O)NC2=O)C3)cc1CC1COCCN1C] | 350 |
| 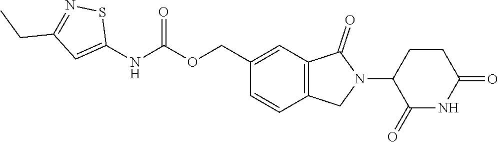 [SMILES: CCc1cc(NC(=O)OCc2ccc3c(c2)C(=O)N(C2CCC(=O)NC2=O)C3)sn1] | 351 |
| 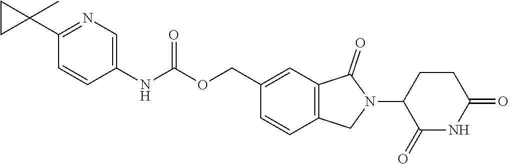 [SMILES: CC1(c2ccc(NC(=O)OCc3ccc4c(c3)C(=O)N(C3CCC(=O)NC3=O)C4)cn2)CC1] | 352 |
| 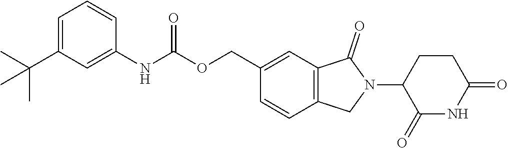 [SMILES: CC(C)(C)c1cccc(NC(=O)OCc2ccc3c(c2)C(=O)N(C2CCC(=O)NC2=O)C3)c1] | 353 |
| 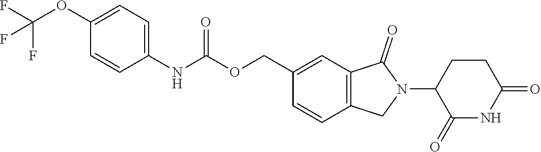 [SMILES: O=C1CCC(N2Cc3ccc(COC(=O)Nc4ccc(OC(F)(F)F)cc4)cc3C2=O)C(=O)N1] | 354 |
| 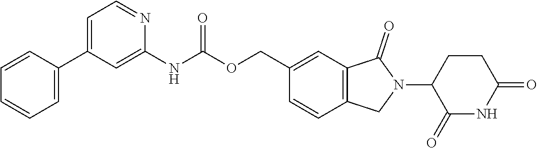 [SMILES: O=C1CCC(N2Cc3ccc(COC(=O)Nc4cc(-c5ccccc5)ccn4)cc3C2=O)C(=O)N1] | 355 |
| 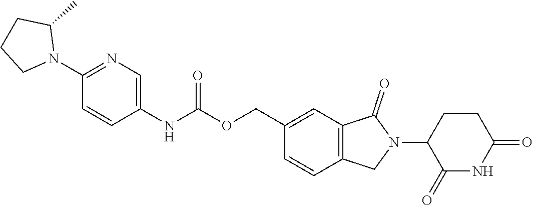 [SMILES: C[C@H]1CCCN1c1ccc(NC(=O)OCc2ccc3c(c2)C(=O)N(C2CCC(=O)NC2=O)C3)cn1] | 356 |
| 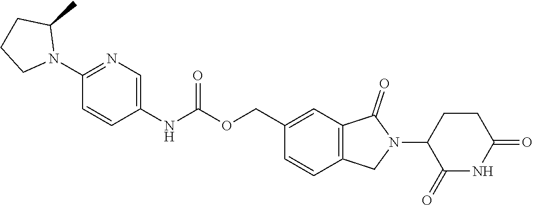 [SMILES: C[C@@H]1CCCN1c1ccc(NC(=O)OCc2ccc3c(c2)C(=O)N(C2CCC(=O)NC2=O)C3)cn1] | 357 |
| 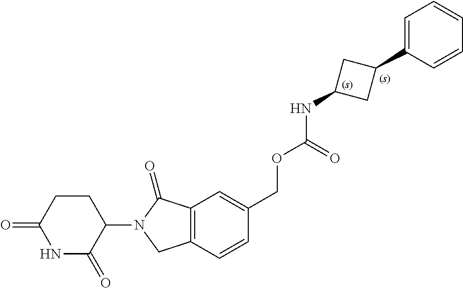 [SMILES: O=C1CCC(N2Cc3ccc(COC(=O)N[C@H]4C[C@@H](c5ccccc5)C4)cc3C2=O)C(=O)N1] | 358 |
| 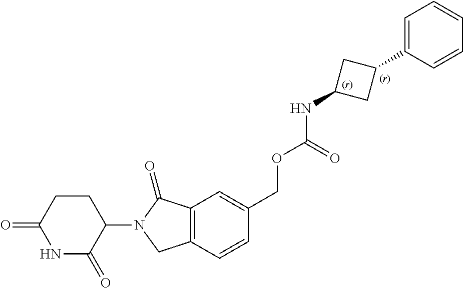 [SMILES: O=C1CCC(N2Cc3ccc(COC(=O)N[C@H]4C[C@H](c5ccccc5)C4)cc3C2=O)C(=O)N1] | 359 |
| 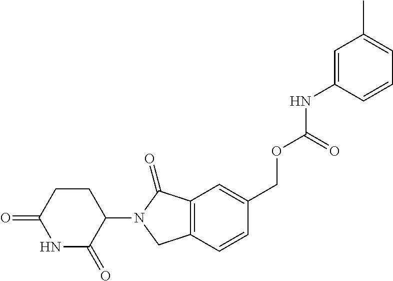 [SMILES: Cc1cccc(NC(=O)OCc2ccc3c(c2)C(=O)N(C2CCC(=O)NC2=O)C3)c1] | 360 |
| 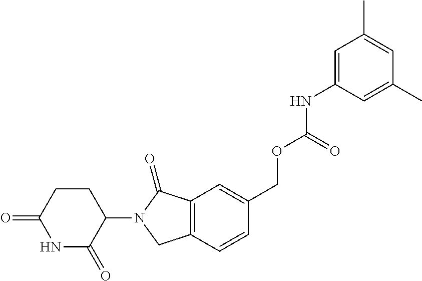 [SMILES: Cc1cc(C)cc(NC(=O)OCc2ccc3c(c2)C(=O)N(C2CCC(=O)NC2=O)C3)c1] | 361 |
| 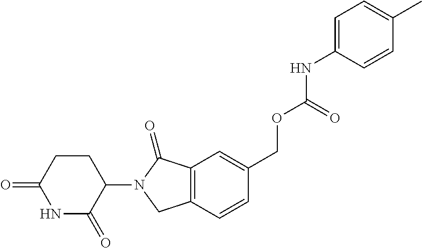 [SMILES: Cc1ccc(NC(=O)OCc2ccc3c(c2)C(=O)N(C2CCC(=O)NC2=O)C3)cc1] | 362 |
| 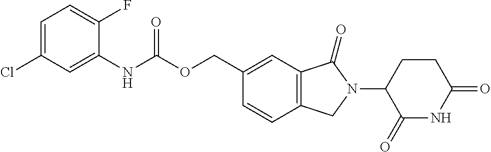 [SMILES: O=C1CCC(N2Cc3ccc(COC(=O)Nc4cc(Cl)ccc4F)cc3C2=O)C(=O)N1] | 363 |
| 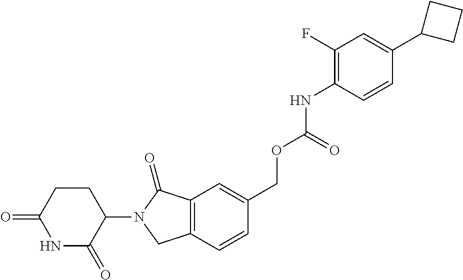 [SMILES: O=C1CCC(N2Cc3ccc(COC(=O)Nc4ccc(C5CCC5)cc4F)cc3C2=O)C(=O)N1] | 364 |
| 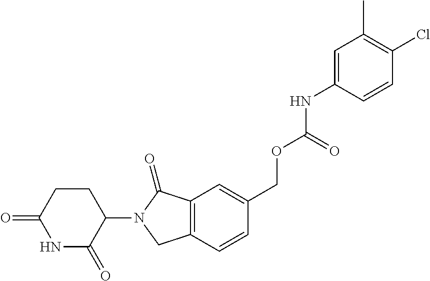 [SMILES: Cc1cc(NC(=O)OCc2ccc3c(c2)C(=O)N(C2CCC(=O)NC2=O)C3)ccc1Cl] | 365 |
| 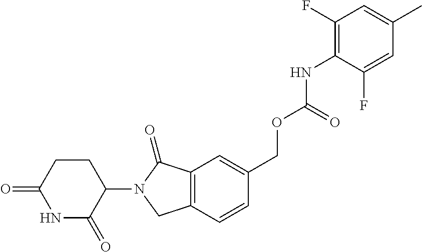 [SMILES: Cc1cc(F)c(NC(=O)OCc2ccc3c(c2)C(=O)N(C2CCC(=O)NC2=O)C3)c(F)c1] | 366 |
| 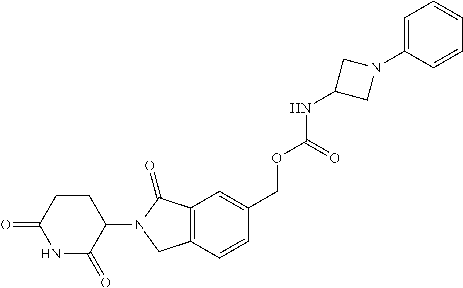 [SMILES: O=C1CCC(N2Cc3ccc(COC(=O)NC4CN(c5ccccc5)C4)cc3C2=O)C(=O)N1] | 367 |
| 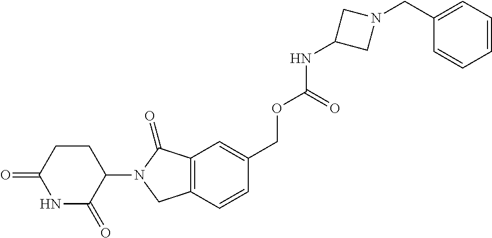 [SMILES: O=C1CCC(N2Cc3ccc(COC(=O)NC4CN(Cc5ccccc5)C4)cc3C2=O)C(=O)N1] | 368 |
| 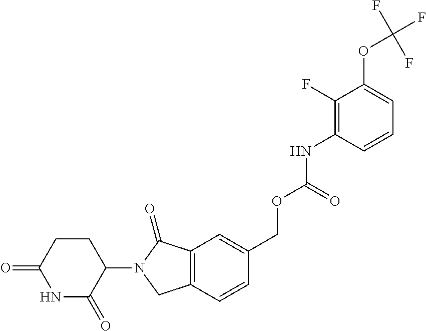 [SMILES: O=C1CCC(N2Cc3ccc(COC(=O)Nc4cccc(OC(F)(F)F)c4F)cc3C2=O)C(=O)N1] | 369 |
| 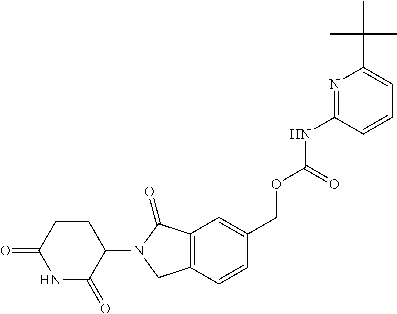 [SMILES: CC(C)(C)c1cccc(NC(=O)OCc2ccc3c(c2)C(=O)N(C2CCC(=O)NC2=O)C3)n1] | 370 |
| 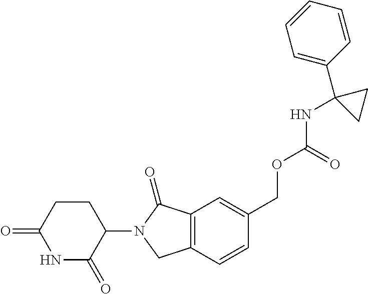 [SMILES: O=C1CCC(N2Cc3ccc(COC(=O)NC4(c5ccccc5)CC4)cc3C2=O)C(=O)N1] | 371 |
| 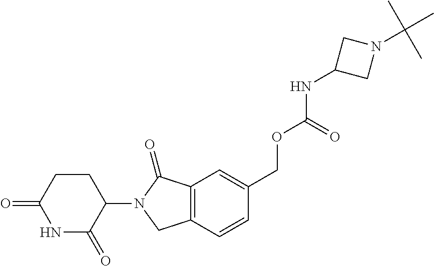 [SMILES: CC(C)(C)N1CC(NC(=O)OCc2ccc3c(c2)C(=O)N(C2CCC(=O)NC2=O)C3)C1] | 372 |
| 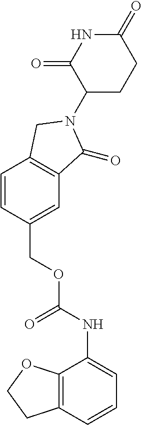 [SMILES: O=C1CCC(N2Cc3ccc(COC(=O)Nc4cccc5c4OCC5)cc3C2=O)C(=O)N1] | 373 |
| 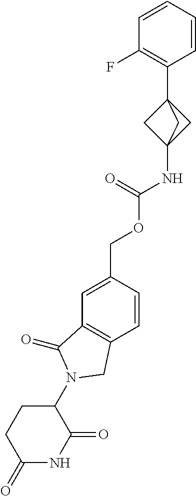 [SMILES: O=C1CCC(N2Cc3ccc(COC(=O)NC45CC(c6ccccc6F)(C4)C5)cc3C2=O)C(=O)N1] | 374 |
| 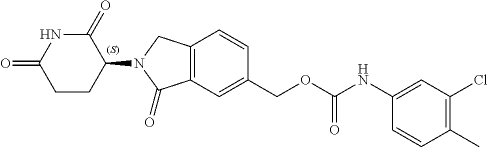 | 375 |
| 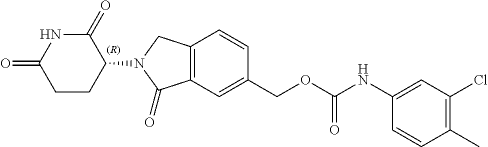 | 376 |
| 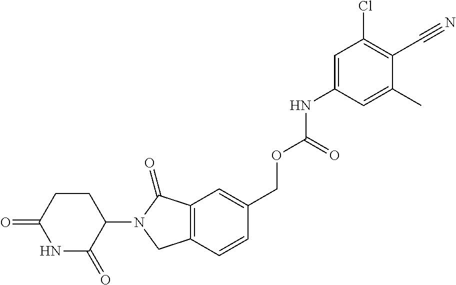 [SMILES: Cc1cc(NC(=O)OCc2ccc3c(c2)C(=O)N(C2CCC(=O)NC2=O)C3)cc(Cl)c1C#N] | 377 |
| 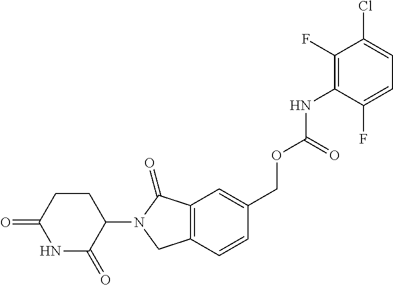 [SMILES: O=C1CCC(N2Cc3ccc(COC(=O)Nc4c(F)ccc(Cl)c4F)cc3C2=O)C(=O)N1] | 378 |
| 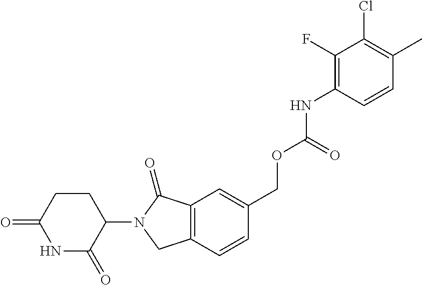 [SMILES: Cc1ccc(NC(=O)OCc2ccc3c(c2)C(=O)N(C2CCC(=O)NC2=O)C3)c(F)c1Cl] | 379 |
| 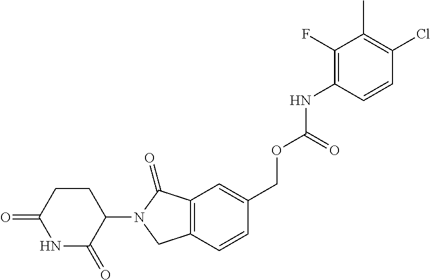 [SMILES: Cc1c(Cl)ccc(NC(=O)OCc2ccc3c(c2)C(=O)N(C2CCC(=O)NC2=O)C3)c1F] | 380 |
| 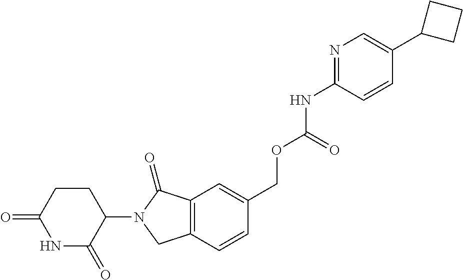 [SMILES: O=C1CCC(N2Cc3ccc(COC(=O)Nc4ccc(C5CCC5)cn4)cc3C2=O)C(=O)N1] | 381 |
| 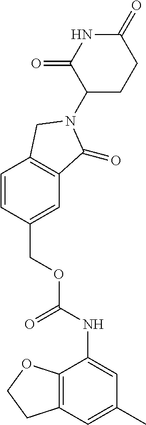 [SMILES: Cc1cc2c(c(NC(=O)OCc3ccc4c(c3)C(=O)N(C3CCC(=O)NC3=O)C4)c1)OCC2] | 382 |
| 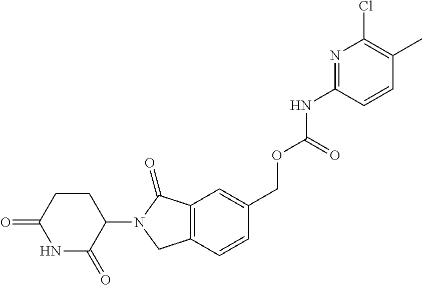 [SMILES: Cc1ccc(NC(=O)OCc2ccc3c(c2)C(=O)N(C2CCC(=O)NC2=O)C3)nc1Cl] | 383 |
| 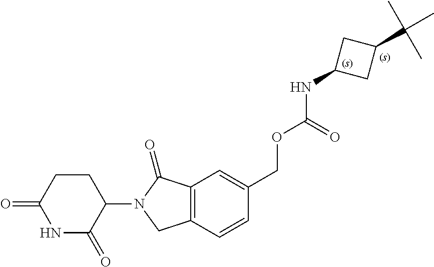 [SMILES: CC(C)(C)[C@H]1C[C@@H](NC(=O)OCc2ccc3c(c2)C(=O)N(C2CCC(=O)NC2=O)C3)C1] | 384 |
| 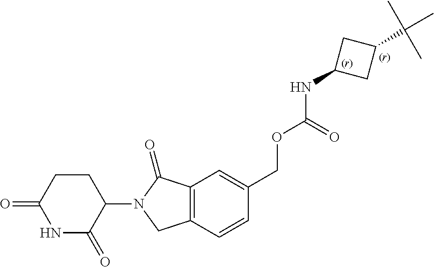 [SMILES: CC(C)(C)[C@H]1C[C@H](NC(=O)OCc2ccc3c(c2)C(=O)N(C2CCC(=O)NC2=O)C3)C1] | 385 |
| 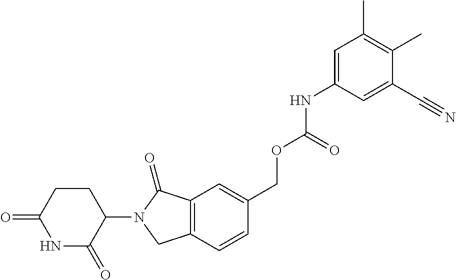 [SMILES: Cc1cc(NC(=O)OCc2ccc3c(c2)C(=O)N(C2CCC(=O)NC2=O)C3)cc(C#N)c1C] | 386 |
| 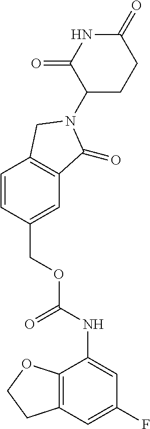 [SMILES: O=C1CCC(N2Cc3ccc(COC(=O)Nc4cc(F)cc5c4OCC5)cc3C2=O)C(=O)N1] | 387 |
| 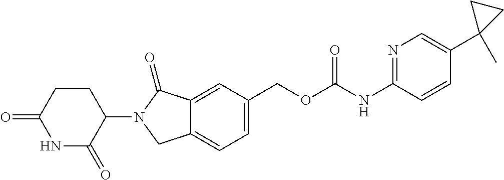 [SMILES: CC1(c2ccc(NC(=O)OCc3ccc4c(c3)C(=O)N(C3CCC(=O)NC3=O)C4)nc2)CC1] | 388 |
| 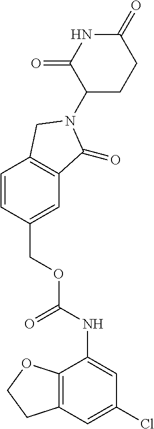 [SMILES: O=C1CCC(N2Cc3ccc(COC(=O)Nc4cc(Cl)cc5c4OCC5)cc3C2=O)C(=O)N1] | 389 |
| 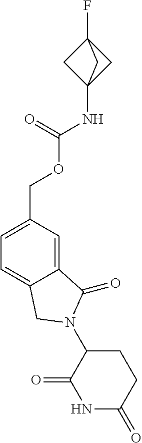 [SMILES: O=C1CCC(N2Cc3ccc(COC(=O)NC45CC(F)(C4)C5)cc3C2=O)C(=O)N1] | 390 |
| 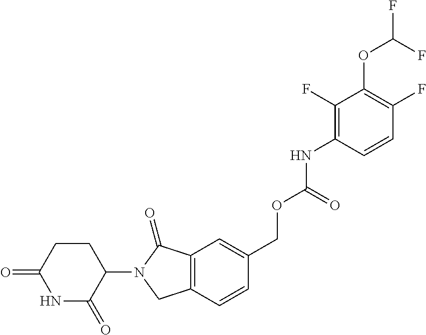 [SMILES: O=C1CCC(N2Cc3ccc(COC(=O)Nc4ccc(F)c(OC(F)F)c4F)cc3C2=O)C(=O)N1] | 391 |
| 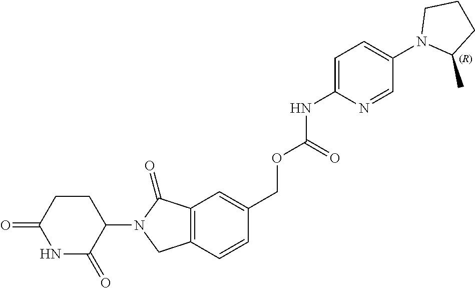 | 392 |
| 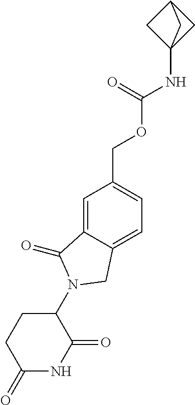 [SMILES: O=C1CCC(N2Cc3ccc(COC(=O)NC45CC(C4)C5)cc3C2=O)C(=O)N1] | 393 |
| 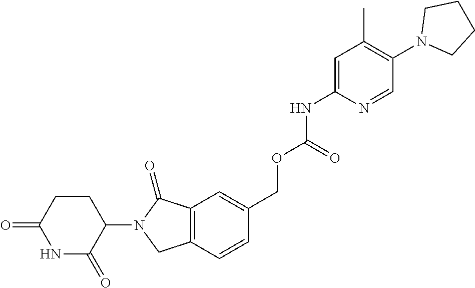 [SMILES: Cc1cc(NC(=O)OCc2ccc3c(c2)C(=O)N(C2CCC(=O)NC2=O)C3)ncc1N1CCCC1] | 394 |
| 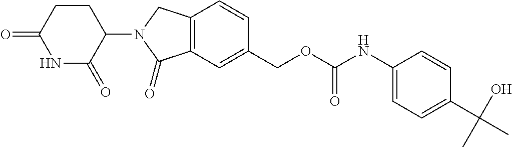 [SMILES: CC(C)(O)c1ccc(NC(=O)OCc2ccc3c(c2)C(=O)N(C2CCC(=O)NC2=O)C3)cc1] | 395 |
| 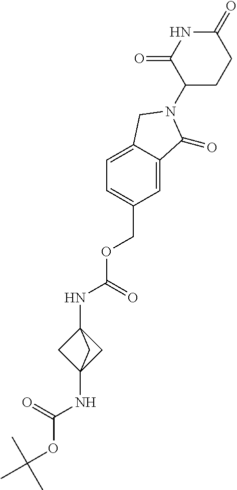 [SMILES: CC(C)(C)OC(=O)NC12CC(NC(=O)OCc3ccc4c(c3)C(=O)N(C3CCC(=O)NC3=O)C4)(C1)C2] | 396 |
| 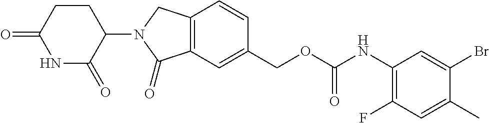 [SMILES: Cc1cc(F)c(NC(=O)OCc2ccc3c(c2)C(=O)N(C2CCC(=O)NC2=O)C3)cc1Br] | 397 |
| 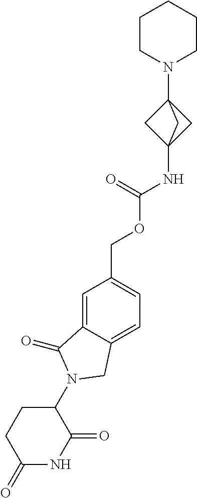 [SMILES: O=C1CCC(N2Cc3ccc(COC(=O)NC45CC(N6CCCCC6)(C4)C5)cc3C2=O)C(=O)N1] | 398 |
| 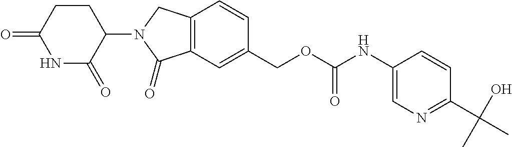 [SMILES: CC(C)(O)c1ccc(NC(=O)OCc2ccc3c(c2)C(=O)N(C2CCC(=O)NC2=O)C3)cn1] | 399 |
| 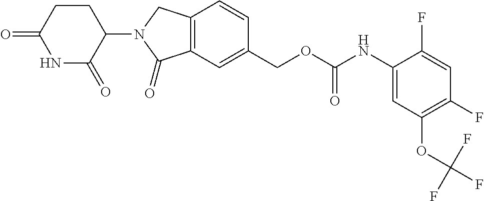 [SMILES: O=C1CCC(N2Cc3ccc(COC(=O)Nc4cc(OC(F)(F)F)c(F)cc4F)cc3C2=O)C(=O)N1] | 400 |
| 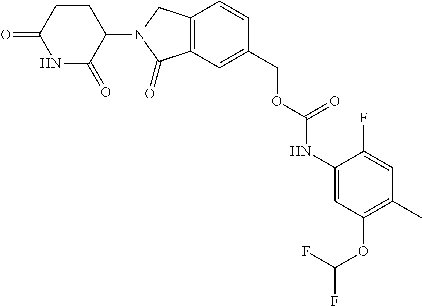 [SMILES: Cc1cc(F)c(NC(=O)OCc2ccc3c(c2)C(=O)N(C2CCC(=O)NC2=O)C3)cc1OC(F)F] | 401 |
| 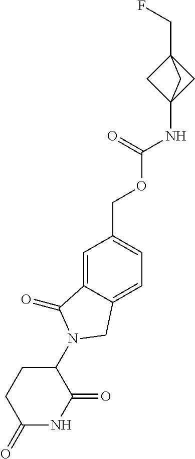 [SMILES: O=C1CCC(N2Cc3ccc(COC(=O)NC45CC(CF)(C4)C5)cc3C2=O)C(=O)N1] | 402 |
| 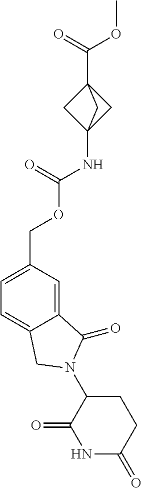 [SMILES: COC(=O)C12CC(NC(=O)OCc3ccc4c(c3)C(=O)N(C3CCC(=O)NC3=O)C4)(C1)C2] | 403 |
| 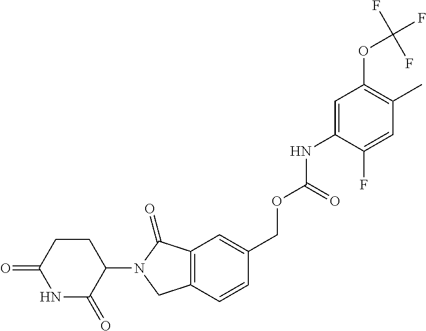 [SMILES: Cc1cc(F)c(NC(=O)OCc2ccc3c(c2)C(=O)N(C2CCC(=O)NC2=O)C3)cc1OC(F)(F)F] | 404 |
| 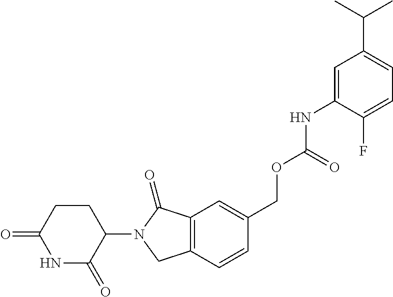 [SMILES: CC(C)c1ccc(F)c(NC(=O)OCc2ccc3c(c2)C(=O)N(C2CCC(=O)NC2=O)C3)c1] | 405 |
| 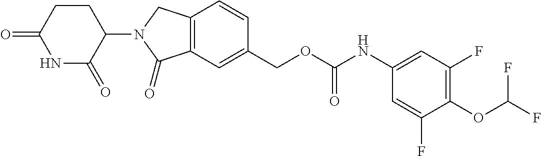 [SMILES: O=C1CCC(N2Cc3ccc(COC(=O)Nc4cc(F)c(OC(F)F)c(F)c4)cc3C2=O)C(=O)N1] | 406 |
| 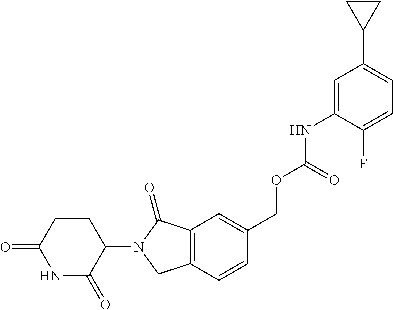 [SMILES: O=C1CCC(N2Cc3ccc(COC(=O)Nc4cc(C5CC5)ccc4F)cc3C2=O)C(=O)N1] | 407 |
| 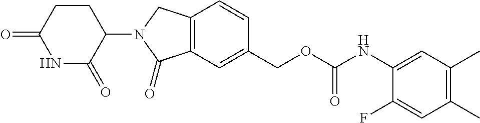 [SMILES: Cc1cc(F)c(NC(=O)OCc2ccc3c(c2)C(=O)N(C2CCC(=O)NC2=O)C3)cc1C] | 408 |
| 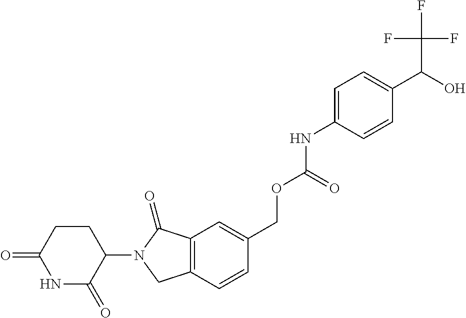 [SMILES: O=C1CCC(N2Cc3ccc(COC(=O)Nc4ccc(C(O)C(F)(F)F)cc4)cc3C2=O)C(=O)N1] | 409 |
| 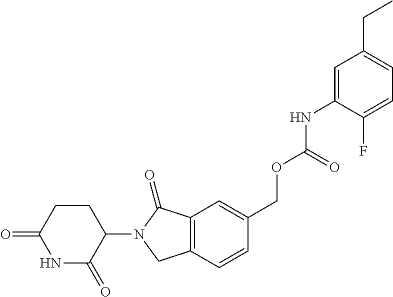 [SMILES: CCc1ccc(F)c(NC(=O)OCc2ccc3c(c2)C(=O)N(C2CCC(=O)NC2=O)C3)c1] | 410 |
| 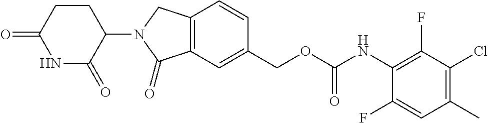 [SMILES: Cc1cc(F)c(NC(=O)OCc2ccc3c(c2)C(=O)N(C2CCC(=O)NC2=O)C3)c(F)c1Cl] | 411 |
| 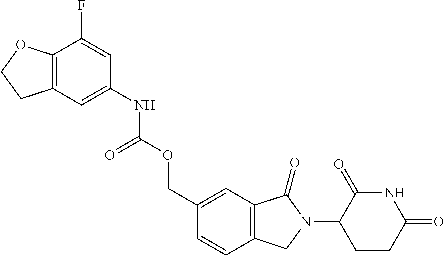 [SMILES: O=C1CCC(N2Cc3ccc(COC(=O)Nc4cc(F)c5c(c4)CCO5)cc3C2=O)C(=O)N1] | 412 |
| 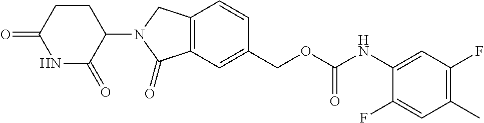 [SMILES: Cc1cc(F)c(NC(=O)OCc2ccc3c(c2)C(=O)N(C2CCC(=O)NC2=O)C3)cc1F] | 413 |
| 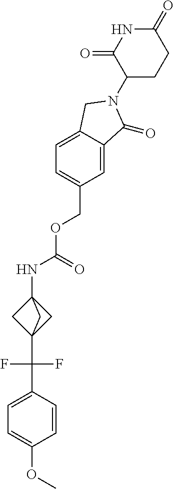 [SMILES: COc1ccc(C(F)(F)C23CC(NC(=O)OCc4ccc5c(c4)C(=O)N(C4CCC(=O)NC4=O)C5)(C2)C3)cc1] | 414 |
| 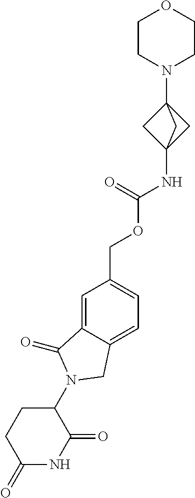 [SMILES: O=C1CCC(N2Cc3ccc(COC(=O)NC45CC(N6CCOCC6)(C4)C5)cc3C2=O)C(=O)N1] | 415 |
| 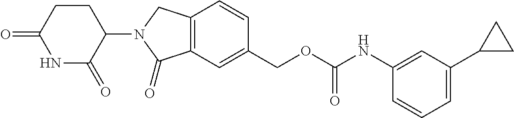 [SMILES: O=C1CCC(N2Cc3ccc(COC(=O)Nc4cccc(C5CC5)c4)cc3C2=O)C(=O)N1] | 416 |
| 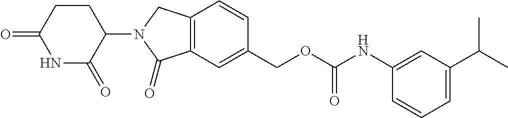 [SMILES: CC(C)c1cccc(NC(=O)OCc2ccc3c(c2)C(=O)N(C2CCC(=O)NC2=O)C3)c1] | 417 |
| 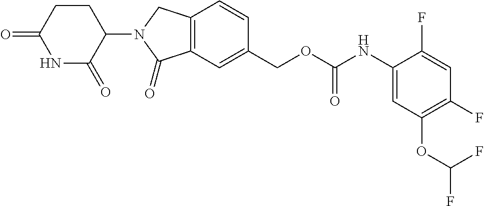 [SMILES: O=C1CCC(N2Cc3ccc(COC(=O)Nc4cc(OC(F)F)c(F)cc4F)cc3C2=O)C(=O)N1] | 418 |
| 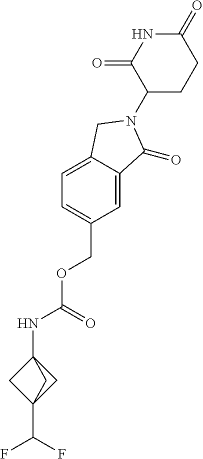 [SMILES: O=C1CCC(N2Cc3ccc(COC(=O)NC45CC(C(F)F)(C4)C5)cc3C2=O)C(=O)N1] | 419 |
| 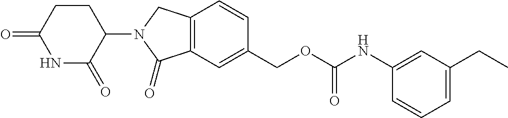 [SMILES: CCc1cccc(NC(=O)OCc2ccc3c(c2)C(=O)N(C2CCC(=O)NC2=O)C3)c1] | 420 |
| 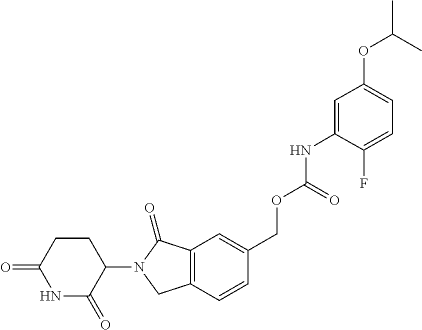 [SMILES: CC(C)Oc1ccc(F)c(NC(=O)OCc2ccc3c(c2)C(=O)N(C2CCC(=O)NC2=O)C3)c1] | 421 |
| 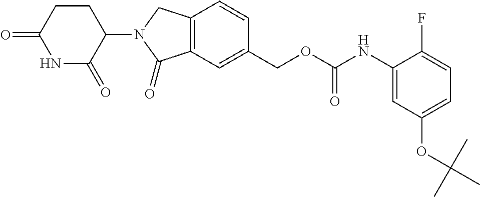 [SMILES: CC(C)(C)Oc1ccc(F)c(NC(=O)OCc2ccc3c(c2)C(=O)N(C2CCC(=O)NC2=O)C3)c1] | 422 |
| 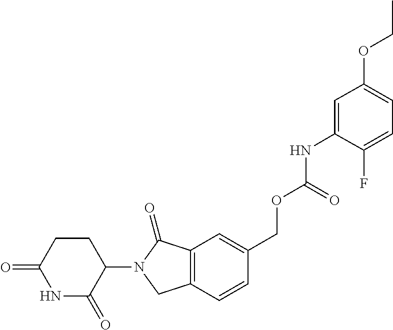 [SMILES: CCOc1ccc(F)c(NC(=O)OCc2ccc3c(c2)C(=O)N(C2CCC(=O)NC2=O)C3)c1] | 423 |
| 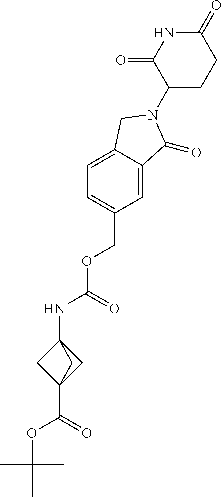 [SMILES: CC(C)(C)OC(=O)C12CC(NC(=O)OCc3ccc4c(c3)C(=O)N(C3CCC(=O)NC3=O)C4)(C1)C2] | 424 |
| 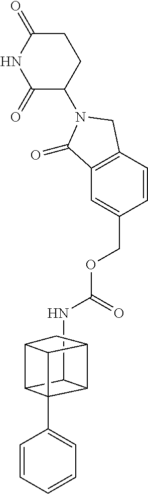 [SMILES: O=C1CCC(N2Cc3ccc(COC(=O)NC4C5C6CC7C6C5(c5ccccc5)C74)cc3C2=O)C(=O)N1] | 425 |
| 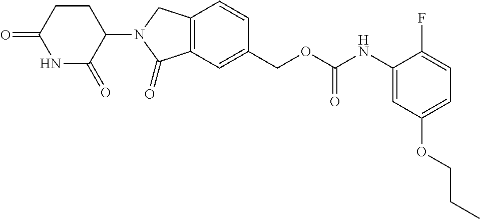 [SMILES: CCCOc1ccc(F)c(NC(=O)OCc2ccc3c(c2)C(=O)N(C2CCC(=O)NC2=O)C3)c1] | 426 |
| 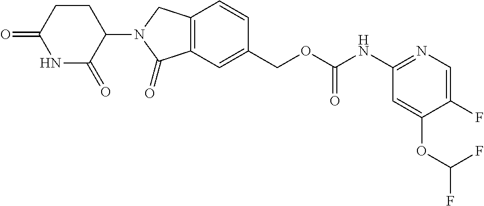 [SMILES: O=C1CCC(N2Cc3ccc(COC(=O)Nc4cc(OC(F)F)c(F)cn4)cc3C2=O)C(=O)N1] | 427 |
| 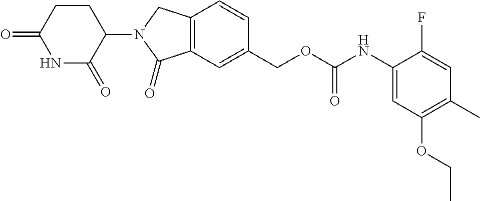 [SMILES: CCOc1cc(NC(=O)OCc2ccc3c(c2)C(=O)N(C2CCC(=O)NC2=O)C3)c(F)cc1C] | 428 |
| 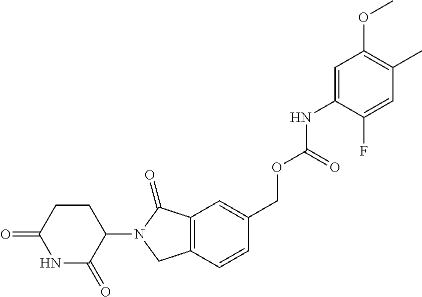 [SMILES: COc1cc(NC(=O)OCc2ccc3c(c2)C(=O)N(C2CCC(=O)NC2=O)C3)c(F)cc1C] | 429 |
| 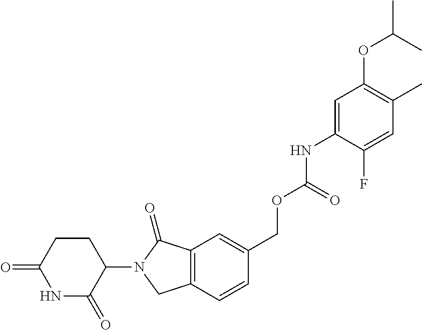 [SMILES: Cc1cc(F)c(NC(=O)OCc2ccc3c(c2)C(=O)N(C2CCC(=O)NC2=O)C3)cc1OC(C)C] | 430 |
| 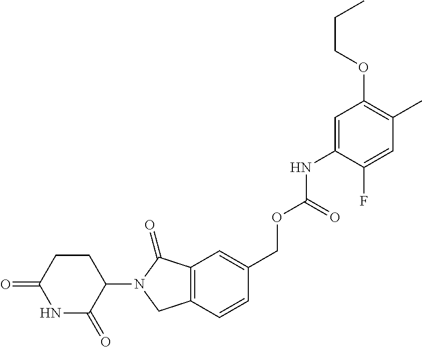 [SMILES: CCCOc1cc(NC(=O)OCc2ccc3c(c2)C(=O)N(C2CCC(=O)NC2=O)C3)c(F)cc1C] | 431 |
| 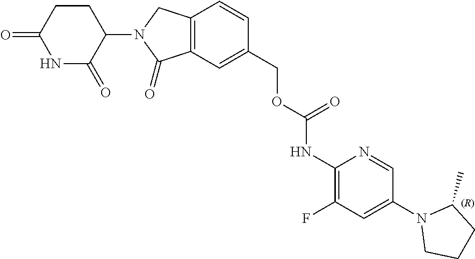 | 432 |
| 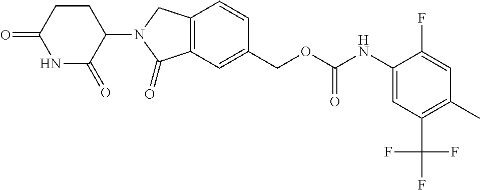 [SMILES: Cc1cc(F)c(NC(=O)OCc2ccc3c(c2)C(=O)N(C2CCC(=O)NC2=O)C3)cc1C(F)(F)F] | 433 |
| 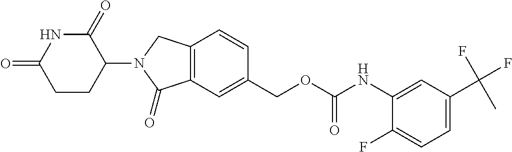 [SMILES: CC(F)(F)c1ccc(F)c(NC(=O)OCc2ccc3c(c2)C(=O)N(C2CCC(=O)NC2=O)C3)c1] | 434 |
| 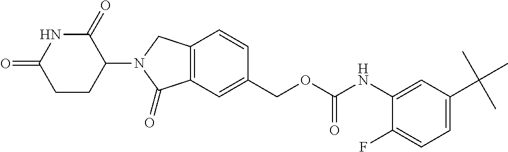 [SMILES: CC(C)(C)c1ccc(F)c(NC(=O)OCc2ccc3c(c2)C(=O)N(C2CCC(=O)NC2=O)C3)c1] | 435 |
| 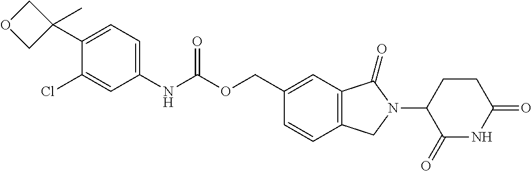 [SMILES: CC1(c2ccc(NC(=O)OCc3ccc4c(c3)C(=O)N(C3CCC(=O)NC3=O)C4)cc2Cl)COC1] | 436 |
| 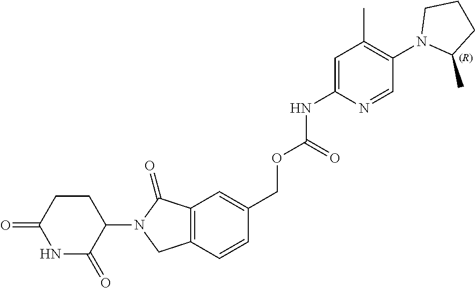 | 437 |
| 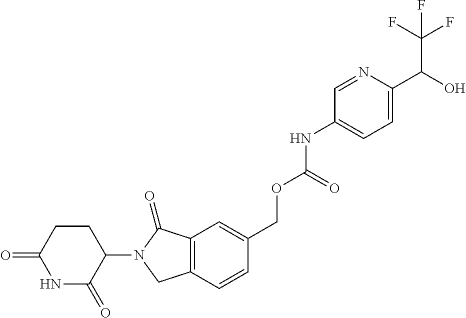 [SMILES: O=C1CCC(N2Cc3ccc(COC(=O)Nc4ccc(C(O)C(F)(F)F)nc4)cc3C2=O)C(=O)N1] | 438 |
| 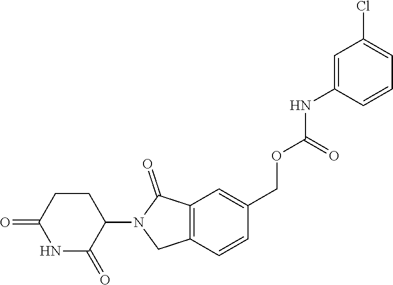 [SMILES: O=C1CCC(N2Cc3ccc(COC(=O)Nc4cccc(Cl)c4)cc3C2=O)C(=O)N1] | 439 |
| 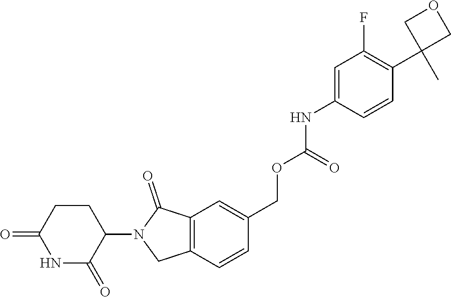 [SMILES: CC1(c2ccc(NC(=O)OCc3ccc4c(c3)C(=O)N(C3CCC(=O)NC3=O)C4)cc2F)COC1] | 440 |

Compounds 201-440 were prepared as follows.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

### General Procedure A:

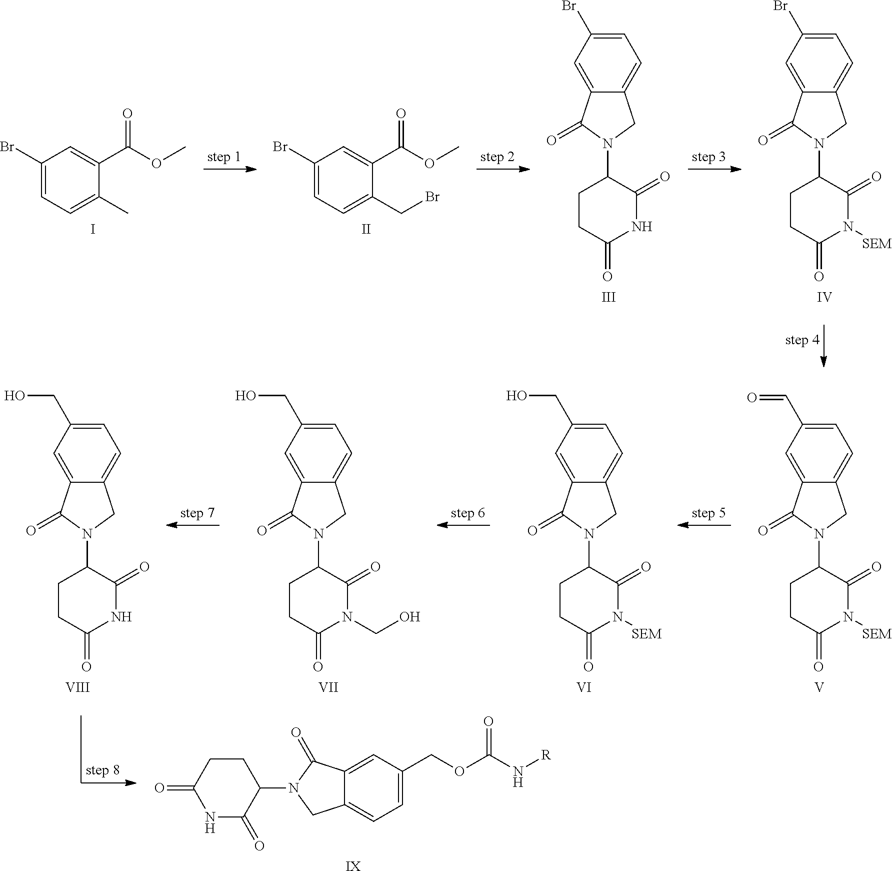

II: To a solution of methyl 5-bromo-2-methylbenzoate 1 (100 g, 436 mmol, 1.00 eq) in trichloromethane (800 mL) was added N-bromosuccinimide (77.6 g, 436 mmol, 1.00 eq) and (E)-2,2′-(diazene-1,2-diyl)bis(2-methylpropanenitrile) (10.5 g, 43.3 mmol, 0.10 eq). The solution was degassed by purging with nitrogen, and the reaction was stirred at 80° C. for 12 h under nitrogen atmosphere. The mixture was concentrated under reduced pressure to give a residue. The residue was purified by silica gel column chromatography (petroleum ether/ethyl acetate=1/0 to 20/1) to afford methyl 5-bromo-2-(bromomethyl)benzoate 1l. ¹H NMR (400 MHz, DMSO-d₆) δ=7.98 (d, J=2.0 Hz, 1H), 7.81-7.79 (m, 1H), 7.57-7.55 (m, 1H), 4.97 (s, 2H), 3.87 (m, 3H).

III: To a solution of methyl 5-bromo-2-(bromomethyl)benzoate 11 (72.5 g, 235 mmol 1.00 eq) and 3-aminopiperidine-2,6-dione hydrochloride (46.6 g, 283 mmol, 1.20 eq, hydrochloride) in acetonitrile (600 mL) was added diisopropylethylamine (123 mL, 706 mmol, 3.00 eq) in one portion under nitrogen. The reaction was stirred at 80° C. for 4 h. After cooling to room temperature, the mixture was concentrated under reduced pressure to give a residue. The residue was triturated with hydrochloric acid (1 M)/ethyl acetate (300 mL/200 mL) at 25° C. for 30 min. The mixture was filtered, and the filter cake was washed with ethyl acetate (100 mL) and dried under reduced pressure to afford 3-(6-bromo-1-oxoisoindolin-2-yl)piperidine-2,6-dione III. ¹H NMR (400 MHz, DMSO-d₆) 5=11.04 (s, 1H), 7.86 (d, J=1.6 Hz, 1H), 7.82 (dd, J=1.8, 8.1 Hz, 1H), 7.60 (d, J=8.1 Hz, 1H), 5.12 (dd, J=5.1, 13.3 Hz, 1H), 4.49-4.28 (m, 2H), 2.99-2.84 (m, 1H), 2.60 (br d, J=17.5 Hz, 1H), 2.39 (dq, J=4.4, 13.2 Hz, 1H), 2.13-1.91 (m, 1H).

IV: To a solution of 3-(6-bromo-1-oxoisoindolin-2-yl)piperidine-2,6-dione III (40.0 g, 123 mmol, 1.00 eq) in dimethylformamide (200 mL) was added 1,8-Diazabicyclo[5.4.0]-7-undecene (48.0 mL, 318 mmol, 2.57 eq) and 2-(chloromethoxy)ethyltrimethylsilane (stabilized with diisopropylethylamine) (40.0 mL, 226 mmol, 1.83 eq) dropwise at 0° C. The reaction was stirred at 25° C. for 6 h. The mixture was diluted with water (500 mL) and ethyl acetate (800 mL). The organic phase was washed with brine (200 mL), dried over anhydrous sodium sulfate, filtered, and concentrated under reduced pressure. The residue was purified by silica gel column chromatography (petroleum ether/ethyl acetate=4/1 to 1/1) to afford 3-(6-bromo-1-oxoisoindolin-2-yl)-1-((2-(trimethylsilyl)ethoxy)methyl)piperidine-2,6-dione IV ¹H NMR (400 MHz, DMSO-d₆) 5=7.90 (d, J=1.6 Hz, 1H), 7.86 (dd, J=2.0, 8.1 Hz, 1H), 7.63 (d, J=8.1 Hz, 1H), 5.26 (dd, J=5.1, 13.4 Hz, 1H), 5.07 (q, J=9.8 Hz, 2H), 4.50 (d, J=17.6 Hz, 1H), 4.36-4.28 (m, 1H), 3.60-3.49 (m, 2H), 3.13-3.01 (m, 1H), 2.86-2.78 (m, 1H), 2.48-2.36 (m, 1H), 2.12-2.05 (m, 1H), 0.90-0.83 (m, 2H), 0.02-−0.01 (m, 9H).

V: To a solution of 3-(6-bromo-1-oxoisoindolin-2-yl)-1-((2-(trimethylsilyl)ethoxy)methyl)piperidine-2,6-dione IV (53.0 g, 117 mmol, 1 eq) in dimethylformamide (300 mL) was added diisopropylethylamine (100 mL, 574 mmol, 4.91 eq), [1,1′-bis(diphenylphosphino)ferrocene]dichloropalladium(II) (8.48 g, 11.6 mmol, 0.10 eq) and triethylsilane (150 mL, 939 mmol, 8.00 eq). The reaction was stirred at 80° C. for 12 h under carbon monoxide atmosphere (50 Psi). The mixture was diluted with ethyl acetate (1.00 L) and water (1.00 L). The organic layer was separated and concentrated under reduced pressure to give a residue. The residue was purified by silica gel column chromatography (petroleum ether/ethyl acetate=1/1) to afford 2-(2,6-dioxo-1-((2-(trimethylsilyl)ethoxy)methyl)piperidin-3-yl)-3-oxoisoindoline-5-carbaldehyde V. ¹H NMR (400 MHz, DMSO-d₆) δ=10.16 (s, 1H), 8.29 (s, 1H), 8.19 (d, J=7.8 Hz, 1H), 7.87 (d, J=7.8 Hz, 1H), 5.30 (br dd, J=4.9, 13.4 Hz, 1H), 5.13-5.03 (m, 2H), 4.65 (br d, J=18.4 Hz, 1H), 4.50-4.40 (m, 1H), 3.62-3.51 (m, 2H), 3.17-3.06 (m, 1H), 2.83 (br d, J=15.8 Hz, 1H), 2.44 (br dd, J=4.1, 13.1 Hz, 1H), 2.15-2.07 (m, 1H), 0.86 (br t, J=7.6 Hz, 2H), 0.00 (s, 9H)

VI: To a solution of 2-(2,6-dioxo-1-((2-(trimethylsilyl)ethoxy)methyl)piperidin-3-yl)-3-oxoisoindoline-5-carbaldehyde V (38.0 g, 94.4 mmol, 1.00 eq) in dimethylformamide (100 mL) and dichloromethane (500 mL) was added sodium triacetoxyborohydride (64.0 g, 302 mmol, 3.20 eq) and acetic acid (27.9 g, 465 mmol, 4.93 eq). The reaction was stirred at 50° C. for 2 h. The mixture was diluted with water (500 mL) and dichloromethane (500 mL). The organic layer was separated and concentrated under reduced pressure to give a residue. The residue was purified by silica gel column chromatography (petroleum ether/ethyl acetate=5/1 to 0/1) to give 3-(6-(hydroxymethyl)-1-oxoisoindolin-2-yl)-1-((2-(trimethylsilyl)ethoxy)methyl) piperidine-2,6-dione VI. ¹H NMR (400 MHz, DMSO-d₆) δ=7.70 (s, 1H), 7.62-7.52 (m, 2H), 5.35 (t, J=5.8 Hz, 1H), 5.29-5.20 (m, 1H), 5.06 (q, J=9.8 Hz, 2H), 4.61 (d, J=5.8 Hz, 2H), 4.47 (br d, J=17.1 Hz, 1H), 4.28 (d, J=16.9 Hz, 1H), 3.61-3.49 (m, 2H), 3.11-3.02 (m, 1H), 2.84-2.77 (m, 1H), 2.40 (br d, J=8.7 Hz, 1H), 2.11-2.03 (m, 1H), 0.89-0.81 (m, 2H), 0.01-−0.03 (m, 9H).

VII: A solution of 3-(6-(hydroxymethyl)-1-oxoisoindolin-2-yl)-1-((2-(trimethylsilyl)ethoxy)methyl)piperidine-2,6-dione VI (17.0 g, 42.0 mmol, 1.00 eq) in hydrochloric acid/dioxane (150 mL) (6 M) was stirred at 50° C. for 2 h. The mixture was concentrated under reduced pressure to afford 1-(hydroxymethyl)-3-(6-(hydroxymethyl)-1-oxoisoindolin-2-yl)piperidine-2,6-dione VII.

VIII: To a solution of 1-(hydroxymethyl)-3-(6-(hydroxymethyl)-1-oxoisoindolin-2-yl)piperidine-2,6-dione VII (13.0 g, 42.7 mmol, 1 eq) in acetonitrile (50.0 mL) was added ammonium hydroxide 30% (0.500 mL, 0.09 eq). The reaction was stirred at 25° C. for 1 h.

The pH was adjusted to pH=5 by addition of 0.5 M hydrochloric acid, and the mixture was concentrated under reduced pressure to give a residue. The residue was purified by reversed phase preparative HPLC to afford 3-(6-(hydroxymethyl)-1-oxoisoindolin-2-yl)piperidine-2,6-dione VIII. ¹H NMR (400 MHz, DMSO-d₆) δ=11.24-10.78 (m, 1H), 7.69 (s, 1H), 7.60-7.53 (m, 2H), 5.35 (t, J=5.8 Hz, 1H), 5.12 (dd, J=5.1, 13.4 Hz, 1H), 4.61 (d, J=5.5 Hz, 2H), 4.49-4.40 (m, 1H), 4.35-4.26 (m, 1H), 2.98-2.86 (m, 1H), 2.66-2.57 (m, 1H), 2.40 (dd, J=4.4, 13.1 Hz, 1H), 2.01 (dtd, J=2.2, 5.2, 12.6 Hz, 1H).

IX: Variant i): To a solution of 3-(6-(hydroxymethyl)-1-oxoisoindolin-2-yl)piperidine-2,6-dione VIII (1.00 eq) in dimethylformamide or acetonitrile (0.04-0.73 M reaction) was added the phenyl carbamate (0.66-1.70 eq) and sodium hydride (60% dispersion in mineral oil) (1.70-3.00 eq) at 0° C. The reaction was stirred at a temperature range of 0 to 25° C. for 0.5-4 h. If necessary, upon completion the reaction was acidified with HCL. If the product precipitated, it was collected by filtration to afford the final carbamate compounds. Otherwise, the mixture was either concentrated to give a residue, or it was extracted and the organic layers were combined, dried over sodium sulfate, filtered, and concentrated to give a residue. The residue was purified by standard methods to afford the final carbamate compounds.

IX: Variant ii): To a solution of 3-(6-(hydroxymethyl)-1-oxoisoindolin-2-yl)piperidine-2,6-dione VIII (1.50 g, 5.47 mmol, 1.00 eq) in dimethylformamide (15.0 mL) was added pyridine (2.21 mL, 27.3 mmol, 5.00 eq) and phenyl chloroformate (1.37 mL, 10.9 mmol, 2.00 eq) at 0° C. The reaction was stirred at 25° C. for 12 h. The mixture was concentrated under reduced pressure to give a residue. The residue was purified by reversed phase preparative HPLC to afford (2-(2,6-dioxopiperidin-3-yl)-3-oxoisoindolin-5-yl)methyl phenyl carbonate. To a solution of (2-(2,6-dioxopiperidin-3-yl)-3-oxoisoindolin-5-yl)methyl phenyl carbonate (1.00 eq) in dimethylformamide (0.1-0.14 M reaction) was added the amine (1.50-5.00 eq). The reaction was stirred at 25° C. for 1-36 h. The mixture was either: diluted to 0.07 M with dimethylformamide and purified by a standard method, or: diluted to 0.10 M with formic acid to give a precipitate which was filtered and dried, or: diluted to 0.10 M with formic acid and purified by a standard method, to afford the final carbamate compounds.

Compound 201: General procedure A with variant i) was used for the preparation from compound VIII employing phenyl (5-chloro-2-methoxy-4-methylphenyl)carbamate. ¹H NMR (400 MHz, DMSO-d₆) δ=11.17-10.81 (m, 1H), 8.87 (s, 1H), 7.82 (s, 1H), 7.75-7.57 (m, 3H), 7.03 (s, 1H), 5.26 (s, 2H), 5.14 (br dd, J=5.1, 13.2 Hz, 1H), 4.53-4.40 (m, 1H), 4.39-4.29 (m, 1H), 3.80 (s, 3H), 2.92 (br s, 1H), 2.65-2.57 (m, 1H), 2.44-2.38 (m, 1H), 2.29 (s, 3H), 2.07-1.97 (m, 1H). MS (ESI) m/z 472.1 [M+H]⁺

Step 1: To a solution of 4-chloro-5-methyl-2-nitrophenol (500 mg, 2.67 mmol, 1.00 eq) in dimethylformamide (3.00 mL) were added potassium carbonate (740 mg, 5.35 mmol, 2.01 eq) and methyl iodide (0.17 mL, 2.73 mmol, 1.02 eq). The reaction was stirred at 25° C. for 2 h. The mixture was extracted with water/ethyl acetate (2.00 mL/2.00 mL). The organic layer was collected and concentrated to afford 1-chloro-4-methoxy-2-methyl-5-nitrobenzene (490 mg, crude) as a yellow solid.

Step 2: To a solution of 1-chloro-4-methoxy-2-methyl-5-nitrobenzene (490 mg, 2.43 mmol, 1.00 eq) in methanol (9.00 mL) and water (3.00 mL) were added iron power (680 mg, 12.1 mmol, 5.01 eq) and ammonium chloride (1.04 g, 19.4 mmol, 8.00 eq). The reaction was stirred at 80° C. for 2 h. The mixture was filtered, and the filtrate was concentrated under reduced pressure to give a concentrated aqueous solution. The solution was extracted with water/ethyl acetate. The organic layers were collected and concentrated under reduced pressure to afford 5-chloro-2-methoxy-4-methylaniline.

Step 3: To a solution of 5-chloro-2-methoxy-4-methylaniline (200 mg, 1.17 mmol, 1.00 eq) in acetonitrile (3.00 mL) was added pyridine (0.47 mL, 5.82 mmol, 4.99 eq), phenyl chloroformate (0.22 mL, 1.75 mmol, 1.50 eq) at 0° C. The reaction was stirred at 0° C. for 2 h. The mixture was concentrated and the obtained residue was purified by standard methods to afford phenyl (5-chloro-2-methoxy-4-methylphenyl)carbamate (690 mg, crude) as an off-white solid.

Compound 202: General procedure A with variant i) was used for the preparation from compound VIII employing phenyl (3-chloro-4-methyl-5-(morpholinomethyl) phenyl)carbamate. ¹H NMR (400 MHz, DMSO-d₆) δ=11.00 (s, 1H), 9.90 (br s, 1H), 8.14 (s, 1H), 7.80 (s, 1H), 7.70-7.62 (m, 2H), 7.55 (s, 1H), 7.43-7.28 (m, 1H), 5.28 (s, 2H), 5.13 (dd, J=5.1, 13.3 Hz, 1H), 4.51-4.44 (m, 1H), 4.39-4.31 (m, 1H), 3.57 (br s, 4H), 3.41 (br s, 2H), 2.96-2.87 (m, 1H), 2.63 (br s, 1H), 2.46-2.32 (m, 5H), 2.29 (s, 3H), 2.06-1.98 (m, 1H). MS (ESI) m/z 541.2 [M+H]⁺

Step 1: To a solution of 2-methyl-5-nitro-benzoic acid (10.0 g, 55.2 mmol, 1.00 eq) in concentrated sulfuric acid (30.0 mL, 98% purity) was added 1,3-dichloro-5,5-dimethyl-imidazolidine-2,4-dione (13.0 g, 66.0 mmol, 1.20 eq). The mixture was stirred at 80° C. for 12 h. After cooling to room temperature, the mixture was poured into ice-water. The resulting white precipitate was collected by filtration and dried under reduced pressure to afford 3-chloro-2-methyl-5-nitro-benzoic acid.

Step 2: To a solution of 3-chloro-2-methyl-5-nitro-benzoic acid (15.0 g, 69.6 mmol, 1.00 eq) in tetrahydrofuran (100 mL) was added borane dimethyl sulfide complex (10 M, 14.0 mL, 140.0 mmol, 2.01 eq). The mixture was stirred at 20° C. for 12 h. The mixture was quenched with methanol (10.0 mL) at 0° C. and then concentrated under reduced pressure to give a residue. The residue was diluted with ethyl acetate (200 mL) and adjusted pH=8 with aqueous sodium bicarbonate (10%, 200 mL). The organic layer was separated and concentrated under reduced pressure to afford (3-chloro-2-methyl-5-nitrophenyl)methanol.

Step 3: To a solution of (3-chloro-2-methyl-5-nitro-phenyl)methanol (11.0 g, 54.5 mmol, 1 eq) (crude) in dichloromethane (200 mL) was added carbon tetrabromide (21 g, 63.32 mmol, 1.16 eq) and triphenylphosphine (18.6 g, 70.9 mmol, 1.30 eq) at 0° C. The mixture was stirred at 20° C. for 1 h. The mixture was concentrated under reduced pressure to give a residue. The residue was triturated with petroleum ether/ethyl acetate=1/1 (200 mL) and filtered. The filtrate was concentrated under reduced pressure to give a residue. The residue was purified by silica gel column chromatography (petroleum ether/ethyl acetate=8/1) to afford 1-(bromomethyl)-3-chloro-2-methyl-5-nitrobenzene.

Step 4: To a solution of 1-(bromomethyl)-3-chloro-2-methyl-5-nitro-benzene (16.0 g, 60.5 mmol, 1.00 eq) in acetonitrile (150 mL) was added morpholine (7.98 mL, 90.7 mmol, 1.50 eq), potassium carbonate (25.0 g, 181 mmol, 2.99 eq) and potassium iodide (1.00 g, 6.05 mmol, 0.100 eq). The reaction was stirred at 80° C. for 12 h. After cooling to room temperature, the mixture was filtered, and the filtrate was concentrated under reduced pressure to give a residue. The residue was purified by silica gel column chromatography (petroleum ether/ethyl acetate=8/1) to give 4-(3-chloro-2-methyl-5-nitrobenzyl)morpholine.

Step 5: To a solution of 4-(3-chloro-2-methyl-5-nitrobenzyl)morpholine (9.70 g, 35.8 mmol, 1.00 eq) in methanol (100 mL) and water (30.0 mL) was added ammonium chloride (15.0 g, 280 mmol, 7.83 eq) and ferrous powder (10.0 g, 179 mmol, 5.00 eq). The reaction was stirred at 80° C. for 2 h. The mixture was filtered, and methanol was removed under reduced pressure. The remaining aqueous solution was extracted with ethyl acetate (2×100 mL). The combined organic layers were washed with brine (20.0 mL), dried over sodium sulfate, filtered, and concentrated under reduced pressure to afford 3-chloro-4-methyl-5-(morpholinomethyl)aniline.

Step 6: To a solution of 3-chloro-4-methyl-5-(morpholinomethyl)aniline (1.00 g, 4.15 mmol, 1.00 eq) and pyridine (1.01 mL, 12.4 mmol, 3.00 eq) in acetonitrile (20.0 mL) was added phenyl chloroformate (0.52 mL, 4.15 mmol, 1.00 eq) at 0° C. The reaction was stirred at 20° C. for 2 h. The mixture was diluted with water (20.0 mL). The resulting precipitate was collected by filtration and dried to afford phenyl (3-chloro-4-methyl-5-(morpholinomethyl)phenyl)carbamate.

Compound 203: General procedure A with variant i) was used for the preparation from compound VIII employing phenyl (3-(trifluoromethoxy)phenyl)carbamate. ¹H NMR (400 MHz, DMSO-d₆) δ=11.00 (s, 1H), 10.15 (s, 1H), 7.81 (s, 1H), 7.72-7.68 (m, 1H), 7.67-7.62 (m, 1H), 7.59 (s, 1H), 7.45-7.39 (m, 2H), 7.03-6.96 (m, 1H), 5.30 (s, 2H), 5.13 (dd, J=5.2, 13.3 Hz, 1H), 4.52-4.44 (m, 1H), 4.39-4.31 (m, 1H), 2.97-2.87 (m, 1H), 2.63 (br d, J=2.4 Hz, 1H), 2.47-2.35 (m, 1H), 2.02 (dtd, J=2.0, 5.1, 12.5 Hz, 1H). MS (ESI) m/z 478.1 [M+H]⁺

Step 1: To a solution of 1-nitro-3-(trifluoromethoxy)benzene (1.00 g, 4.83 mmol, 1.00 eq) in methanol (15.0 mL) and water (5.00 mL) was added ammonium chloride (2.07 g, 38.6 mmol, 8.00 eq) and iron power (1.35 g, 24.1 mmol, 5.00 eq). The reaction was stirred at 80° C. for 2 h. The mixture was filtered, and the solvents were mostly removed under reduced pressure to give a concentrated solution. The solution was triturated with ethyl acetate/water (20.0 ml/10.0 ml), and a drop of saturated sodium bicarbonate solution was added. The organic layer was collected and concentrated under reduced pressure to afford 3-(trifluoromethoxy)aniline.

Step 2: To a solution of 3-(trifluoromethoxy)aniline (0.15 mL, 1.10 mmol, 1.00 eq) in acetonitrile (5.00 mL) was added pyridine (0.44 mL, 5.50 mmol, 5.00 eq). The reaction was stirred for 0.5 h, then phenyl chloroformate (0.16 mL, 1.32 mmol, 1.20 eq) was added at 0° C. The reaction was stirred for 1.5 h at 0° C. The mixture was concentrated under reduced pressure to give a residue. The residue was diluted with water (10.0 ml) and ethyl acetate (30.0 mL). The organic layer was collected and concentrated and the obtained residue was purified by standard methods to afford phenyl (3-(trifluoromethoxy)phenyl)carbamate.

Compound 204: General procedure A with variant i) was used for the preparation from compound VIII employing phenyl (3-(morpholinomethyl)-5-(trifluoromethoxy)phenyl)carbamate. ¹H NMR (400 MHz, DMSO-d₆) δ=10.99 (s, 1H), 10.11 (s, 1H), 7.80 (s, 1H), 7.71-7.60 (m, 2H), 7.52-7.37 (m, 2H), 6.91 (s, 1H), 5.29 (s, 2H), 5.18-5.07 (m, 1H), 4.53-4.29 (m, 2H), 3.62-3.52 (m, 4H), 3.45 (s, 2H), 2.98-2.85 (m, 1H), 2.60 (br d, J=18.1 Hz, 1H), 2.43-2.38 (m, 1H), 2.34 (br s, 4H), 2.05-1.96 (m, 1H). MS (ESI) m/z 577.1 [M+H]⁺

Step 1: To a solution of 3-hydroxy-5-nitro-benzoic acid (4.50 g, 24.6 mmol, 1.00 eq) and morpholine (2.57 g, 29.5 mmol, 2.60 mL, 1.2 eq) in dichloromethane (100 mL) was added O-(7-azabenzotriazol-1-yl)-N,N,N,N-tetramethyluronium hexafluorophosphate (11.2 g, 29.5 mmol, 1.20 eq) and triethylamine (5.06 mL, 49.2 mmol, 20.00 eq). The reaction was stirred at 20° C. for 12 h. The mixture was diluted with water (150 mL) and extracted with ethyl acetate (3×100 mL). The combined organic layers were washed with brine (100 mL), dried over sodium sulfate, filtered, and concentrated under reduced pressure to give a residue. The residue was purified by silica gel column chromatography (petroleum ether/ethyl acetate=0/1) to afford (3-hydroxy-5-nitrophenyl)(morpholino) methanone.

Step 2: To a solution of (3-hydroxy-5-nitro-phenyl)-morpholino-methanone (2.40 g, 9.52 mmol, 1.00 eq) in toluene (200 mL) were added trimethyl(trifluoromethyl)silane (6.77 g, 47.6 mmol, 5.00 eq), silver trifluoromethanesulfonate (12.2 g, 47.6 mmol, 5.00 eq), 2-fluoropyridine (4.09 mL, 47.6 mmol, 5.00 eq), caesium fluoride (2.10 mL, 57.1 mmol, 6.00 eq), N-fluorobenzenesulfonimide (6.00 g, 19.0 mmol, 2.00 eq) and 1-(chloromethyl)-4-fluoro-1,4-diazoniabicyclo(2.2.2)octane bis(tetrafluoroborate) (6.74 g, 19.0 mmol, 2.00 eq). The reaction was stirred at 25° C. under a nitrogen atmosphere for 12 h. The mixture was filtered, and the filtrate was concentrated under reduced pressure to give a residue. The residue was diluted with water (100 mL) and extracted with ethyl acetate (3×100 mL). The combined organic layers were washed with brine (100 mL), dried over anhydrous sodium sulfate, filtered, and the filtrate was concentrated to give a residue. The residue was purified by silica gel column chromatography (petroleum ether/ethyl acetate=1/1) to afford morpholino(3-nitro-5-(trifluoromethoxy) phenyl)methanone.

Step 3: To a solution of morpholino(3-nitro-5-(trifluoromethoxy)phenyl)methanone (2.60 g, 8.12 mmol, 1.00 eq) in tetrahydrofuran (30.0 mL) was added borane dimethyl sulfide complex (10.0 M, 1.62 mL, 2.00 eq) dropwise at 0° C. under nitrogen atmosphere. The reaction was stirred at 60° C. for 4 h. The mixture was quenched with methanol (9.00 mL) and concentrated to give a residue. The residue was purified by reversed phase preparative HPLC to afford 4-(3-nitro-5-(trifluoromethoxy)benzyl)morpholine.

Step 4: To a solution of 4-(3-nitro-5-(trifluoromethoxy)benzyl)morpholine (1.10 g, 3.59 mmol, 1.00 eq) in methanol (50.0 mL) and water (50.0 mL) were added iron powder (1.40 g, 25.14 mmol, 7.00 eq) and ammonium chloride (1.34 g, 25.14 mmol, 7.00 eq). The reaction was stirred at 80° C. for 3 h. The mixture was filtered over Celite, and the filtrate was concentrated under reduced pressure to afford 3-(morpholinomethyl)-5-(trifluoromethoxy)aniline.

Step 5: To a solution of 3-(morpholinomethyl)-5-(trifluoromethoxy)aniline (580 mg, 2.10 mmol, 1.00 eq) in acetonitrile (3.00 mL) was added phenyl chloroformate (0.39 mL, 3.15 mmol, 1.50 eq) and pyridine (0.5 mL, 6.30 mmol, 3.00 eq) at 0° C. The reaction was stirred at 25° C. for 12 h. The mixture was diluted with water (100 mL) and extracted with ethyl acetate (3×30.0 mL). The combined organic layers were washed with brine (30 mL), dried over anhydrous sodium sulfate, filtered, and the filtrate was concentrated and the obtained residue was purified by standard methods to afford phenyl (3-(morpholinomethyl)-5-(trifluoromethoxy)phenyl)carbamate.

Compound 205: General procedure A with variant i) was used for the preparation from compound VIII employing phenyl (3-(difluoromethoxy)phenyl)carbamate. ¹H NMR (400 MHz, DMSO-d₆) δ=11.00 (br s, 1H), 10.05 (s, 1H), 7.80 (s, 1H), 7.71-7.67 (m, 1H), 7.67-7.62 (m, 1H), 7.40 (br s, 1H), 7.37 (s, 1H), 7.36-7.28 (m, 2H), 7.19 (t, J=76 Hz, 1H), 6.81 (br d, J=7.5 Hz, 1H), 5.29 (s, 2H), 5.13 (dd, J=5.1, 13.3 Hz, 1H), 4.52-4.44 (m, 1H), 4.39-4.30 (m, 1H), 2.97-2.87 (m, 1H), 2.64-2.58 (m, 1H), 2.48-2.37 (m, 1H), 2.07-1.98 (m, 1H). MS (ESI) m/z 460.1 [M+H]⁺

Step 1: To a solution of 1-(difluoromethoxy)-3-nitrobenzene (2.00 g, 10.6 mmol, 1.00 eq) in methanol (15.0 mL) and water (5.00 mL) was added iron powder (2.95 g, 52.9 mmol, 5.00 eq) and ammonium chloride (4.53 g, 84.6 mmol, 8.00 eq). The reaction was stirred at 80° C. for 2 h. The reaction solution was filtered, and the filtrate was extracted with ethyl acetate. The combined organic layers were washed with brine, dried with anhydrous sodium sulfate, filtered, and concentrated under reduced pressure to afford 3-(difluoromethoxy)aniline.

Step 2: To a solution of 3-(difluoromethoxy)aniline (1.00 g, 6.28 mmol, 1.00 eq) and pyridine (2.54 mL, 31.4 mmol, 5.00 eq) in acetonitrile (5.00 mL) was added phenyl chloroformate (0.94 mL, 7.54 mmol, 1.20 eq). The mixture was stirred at 25° C. for 3 h. The mixture was concentrated and the obtained residue was purified by standard methods to afford phenyl (3-(difluoromethoxy)phenyl)carbamate.

Compound 206: General procedure A with variant i) was used for the preparation from compound VIII employing phenyl(5-chloro-6-methylpyridin-3-yl)carbamate. ¹H NMR (400 MHz, DMSO-d₆) δ=11.00 (s, 1H), 10.18 (br s, 1H), 8.50-8.42 (m, 1H), 7.99 (s, 1H), 7.80 (s, 1H), 7.72-7.66 (m, 1H), 7.66-7.62 (m, 1H), 5.30 (s, 2H), 5.17-5.08 (m, 1H), 4.52-4.43 (m, 1H), 4.38-4.30 (m, 1H), 2.98-2.85 (m, 1H), 2.65-2.56 (m, 1H), 2.46 (s, 3H), 2.44-2.34 (m, 1H), 2.05-1.95 (m, 1H). MS (ESI) m/z 443.2 [M+H]⁺

Step 1: To a solution of 3-chloro-2-methyl-5-nitropyridine (500 mg, 2.90 mmol, 1.00 eq) in methanol (5.00 mL) and water (5.00 mL) were added iron powder (1.13 g, 20.3 mmol, 7.00 eq) and ammonium chloride (1.08 g, 20.3 mmol, 7.00 eq). The reaction was stirred at 80° C. for 2 h. The mixture was filtered over Celite, and the filtrate was concentrated under reduced pressure to afford 5-chloro-6-methylpyridin-3-amine.

Step 2: To a solution of 5-chloro-6-methylpyridin-3-amine (200 mg, 1.40 mmol, 1.00 eq) in acetonitrile (2.00 mL) were added phenyl chloroformate (0.26 mL, 2.10 mmol, 1.50 eq) and pyridine (0.34 mL, 4.21 mmol, 3.00 eq). The reaction was stirred at 25° C. for 12 h. The mixture was diluted with ethyl acetate (30.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (5-chloro-6-methylpyridin-3-yl)carbamate.

Compound 207: General procedure A with variant i) was used for the preparation from compound VIII employing phenyl (2,6-dimethylpyridin-4-yl)carbamate. ¹H NMR (400 MHz, DMSO-d₆) δ=10.99 (s, 1H), 10.10 (s, 1H), 8.18 (s, 1H), 7.80 (s, 1H), 7.71-7.60 (m, 2H), 7.12 (s, 2H), 5.29 (s, 2H), 5.12 (dd, J=5.1, 13.3 Hz, 1H), 4.51-4.43 (m, 1H), 4.39-4.30 (m, 1H), 2.91 (ddd, J=5.4, 13.7, 17.4 Hz, 1H), 2.64-2.57 (m, 1H), 2.40 (br dd, J=4.6, 13.1 Hz, 1H), 2.33 (s, 6H), 2.07-1.97 (m, 1H). MS (ESI) m/z 423.1 [M+H]⁺

To a solution of 2,6-dimethylpyridin-4-amine (1.00 g, 8.19 mmol, 1.00 eq) in acetonitrile (20.0 mL) was added pyridine (3.30 mL, 40.9 mmol, 5.00 eq) and phenyl chloroformate (1.54 mL, 12.2 mmol, 1.50 eq) at 0° C. The reaction was stirred at 25° C. for 0.5 h. The mixture was concentrated and the obtained residue was purified by standard methods to afford phenyl (2,6-dimethylpyridin-4-yl)carbamate.

Compound 208: General procedure A with variant i) was used for the preparation from compound VIII employing phenyl (3,5-dimethylphenyl)carbamate. ¹H NMR (400 MHz, DMSO-d₆) δ=10.9 (br s, 1H), 9.65 (s, 1H), 7.78 (s, 1H), 7.70-7.59 (m, 2H), 7.09 (s, 2H), 6.64 (s, 1H), 5.25 (s, 2H), 5.13 (dd, J=5.1, 13.4 Hz, 1H), 4.52-4.42 (m, 1H), 4.39-4.28 (m, 1H), 2.99-2.84 (m, 1H), 2.60 (td, J=2.1, 15.3 Hz, 1H), 2.40 (br dd, J=4.4, 13.1 Hz, 1H), 2.21 (s, 6H), 2.06-1.98 (m, 1H). MS (ESI) m/z 422.1 [M+H]⁺

To a solution of 3,5-dimethylaniline (0.51 mL, 4.13 mmol, 1.00 eq) in acetonitrile (10.00 mL) was added pyridine (1.67 mL, 20.6 mmol, 5.00 eq) and phenyl chloroformate (1.03 mL, 8.25 mmol, 2.00 eq) at 0° C. in portions. The reaction was stirred at 25° C. for 1 h. The mixture was concentrated under reduced pressure to give a residue, which was purified by silica gel column chromatography (petroleum ether/ethyl acetate=1/0 to 3/1) to afford phenyl (3,5-dimethylphenyl) carbamate.

Compound 209: General procedure A with variant i) was used for the preparation from compound VIII employing phenyl (3-chloro-4-fluorophenyl)carbamate. ¹H NMR (400 MHz, DMSO-d₆) δ=10.99 (br s, 1H), 10.05 (br s, 1H), 7.79 (s, 1H), 7.74-7.61 (m, 3H), 7.44-7.31 (m, 2H), 5.28 (s, 2H), 5.12 (dd, J=5.1, 13.3 Hz, 1H), 4.52-4.41 (m, 1H), 4.39-4.29 (m, 1H), 2.98-2.85 (m, 1H), 2.60 (br dd, J=2.1, 15.4 Hz, 1H), 2.42 (dt, J=4.4, 13.3 Hz, 1H), 2.05-1.96 (m, 1H). MS (ESI) m/z 446.1 [M+H]⁺

To a solution of 3-chloro-4-fluoroaniline (1.00 g, 6.87 mmol, 1.00 eq) in acetonitrile (10.0 mL) was added pyridine (2.77 mL, 34.4 mmol, 5.00 eq) and phenyl chloroformate (1.72 mL, 13.7 mmol, 2.00 eq) in portions at 0° C. The reaction was stirred at 25° C. for 0.5 h. The mixture was concentrated and the obtained residue was purified by standard methods to afford phenyl (3-chloro-4-fluorophenyl)carbamate.

Compound 210: General procedure A with variant i) was used for the preparation from compound VIII employing phenyl (3-chloro-4-methylphenyl)carbamate. ¹H NMR (400 MHz, DMSO-d₆) δ=11.00 (s, 1H), 9.93 (s, 1H), 7.80 (s, 1H), 7.74-7.54 (m, 3H), 7.37-7.19 (m, 2H), 5.28 (s, 2H), 5.13 (dd, J=5.1, 13.2 Hz, 1H), 4.53-4.44 (m, 1H), 4.38-4.31 (m, 1H), 2.98-2.87 (m, 1H), 2.61 (br d, J=17.6 Hz, 1H), 2.41 (br dd, J=4.4, 13.2 Hz, 1H), 2.26 (s, 3H), 2.06-1.97 (m, 1H). MS (ESI) m/z 442.1 [M+H]⁺

Preparation of phenyl (3-chloro-4-methylphenyl)carbamate: To a solution of 3-chloro-4-methylaniline (5.00 g, 35.3 mmol, 1.00 eq) in acetonitrile (50.0 mL) was added pyridine (5.70 mL, 70.6 mmol, 2.00 eq) and phenyl chloroformate (4.87 mL, 38.8 mmol, 1.10 eq) at 0° C. The mixture was stirred at 30° C. for 2 h. The mixture was concentrated and the obtained residue was purified by standard methods to afford phenyl (3-chloro-4-methylphenyl)carbamate.

Compound 211: General procedure A with variant i) was used for the preparation from compound VIII employing phenyl (3-chloro-5-(trifluoromethoxy)phenyl)carbamate. ¹H NMR (400 MHz, DMSO-d₆) 5=11.00 (s, 1H), 10.35 (s, 1H), 7.81 (s, 1H), 7.73-7.68 (m, 1H), 7.67-7.62 (m, 1H), 7.60-7.49 (m, 2H), 7.17 (s, 1H), 5.31 (s, 2H), 5.13 (dd, J=5.1, 13.3 Hz, 1H), 4.53-4.44 (m, 1H), 4.39-4.31 (m, 1H), 2.99-2.85 (m, 1H), 2.64-2.58 (m, 1H), 2.41 (dd, J=4.4, 13.0 Hz, 1H), 2.07-1.95 (m, 1H). MS (ESI) m/z 332.0 [M+H]⁺

To a solution of 3-chloro-5-(trifluoromethoxy)aniline (150 mg, 708 μmol, 1.00 eq) in acetonitrile (1.00 mL) were added pyridine (0.29 mL, 3.54 mmol, 5.00 eq) and phenyl chloroformate (0.11 mL, 850 μmol, 1.20 eq) at 0° C. The reaction was stirred at 0° C. for 2 h. The mixture was filtered to give a solution, which was purified by standard methods to afford phenyl (3-chloro-5-(trifluoromethoxy)phenyl)carbamate.

Compound 212: General procedure A with variant i) was used for the preparation from compound VIII employing phenyl (3-chloro-5-fluorophenyl)carbamate. ¹H NMR (400 MHz, DMSO-d₆) δ=10.99 (s, 1H), 10.25 (s, 1H), 7.80 (s, 1H), 7.74-7.56 (m, 2H), 7.44-7.28 (m, 2H), 7.10-6.96 (m, 1H), 5.30 (s, 2H), 5.18-5.06 (m, 1H), 4.54-4.42 (m, 1H), 4.40-4.31 (m, 1H), 2.98-2.86 (m, 1H), 2.62 (br d, J=2.6 Hz, 1H), 2.46-2.37 (m, 1H), 2.05-1.96 (m, 1H). MS (ESI) m/z 446.0 [M+H]⁺

To a solution of 3-chloro-5-fluoroaniline (1.00 g, 6.87 mmol, 1.00 eq) in acetonitrile (20.0 mL) were added pyridine (2.77 mL, 34.3 mmol, 5.00 eq) and phenyl chloroformate (1.29 mL, 10.3 mmol, 1.50 eq) at 0° C. The reaction was stirred at 25° C. for 1 h. The mixture was concentrated and the obtained residue was purified by standard methods to afford phenyl (3-chloro-5-fluorophenyl)carbamate.

Compound 213: General procedure A with variant i) was used for the preparation from compound VIII employing phenyl (3-(2-oxa-5-azabicyclo[2.2.1]heptan-5-ylmethyl)-5-chloro-4-methylphenyl)carbamate.

¹H NMR (400 MHz, DMSO-d₆) δ=11.00 (s, 1H), 9.89 (br s, 1H), 8.14 (s, 1H), 7.80 (s, 1H), 7.71-7.62 (m, 2H), 7.54 (br s, 1H), 7.41 (d, J=1.5 Hz, 1H), 5.27 (s, 2H), 5.13 (dd, J=5.0, 13.3 Hz, 1H), 4.46 (s, 1H), 4.37 (s, 2H), 3.91 (s, 1H), 3.72-3.64 (m, 2H), 3.54 (dd, J=1.6, 7.4 Hz, 1H), 3.46 (br s, 1H), 2.97-2.87 (m, 1H), 2.75 (br d, J=9.0 Hz, 1H), 2.61 (br d, J=17.0 Hz, 1H), 2.47 (br d, J=10.3 Hz, 1H), 2.41 (br dd, J=4.6, 13.0 Hz, 1H), 2.26 (s, 3H), 2.07-1.97 (m, 1H), 1.80 (br s, 1H), 1.61 (br d, J=9.3 Hz, 1H). MS (ESI) m/z 553.2 [M+H]⁺

To a solution of 3-(2-oxa-5-azabicyclo[2.2.1]heptan-5-ylmethyl)-5-chloro-4-methylaniline (350 mg, 1.38 mmol, 1.00 eq) in acetonitrile (2.00 mL) were added pyridine (0.56 mL, 6.92 mmol, 5.00 eq) and phenyl chloroformate (0.21 mL, 1.66 mmol, 1.20 eq) at 0° C. The reaction was stirred at 0° C. for 2 h. The mixture was concentrated and the obtained residue was purified by standard methods to afford phenyl (3-(2-oxa-5-azabicyclo[2.2.1]heptan-5-ylmethyl)-5-chloro-4-methylphenyl)carbamate.

Compound 214: General procedure A with variant i) was used for the preparation from compound VIII employing phenyl(5-chloro-2-methoxyphenyl)carbamate.

¹H NMR (400 MHz, DMSO-d₆) δ=10.99 (br s, 1H), 8.95 (s, 1H), 7.82 (s, 1H), 7.77 (br d, J=1.5 Hz, 1H), 7.72-7.66 (m, 1H), 7.65-7.60 (m, 1H), 7.13-7.08 (m, 1H), 7.06-7.01 (m, 1H), 5.27 (s, 2H), 5.13 (dd, J=5.1, 13.3 Hz, 1H), 4.51-4.43 (m, 1H), 4.37-4.30 (m, 1H), 3.80 (s, 3H), 2.97-2.86 (m, 1H), 2.60 (td, J=2.1, 15.3 Hz, 1H), 2.40 (br dd, J=4.5, 13.1 Hz, 1H), 2.05-1.97 (m, 1H). MS (ESI) m/z 458.1 [M+H]⁺

To a solution of 5-chloro-2-methoxyaniline (1.00 g, 6.35 mmol, 1.00 eq) in acetonitrile (10.0 mL) were added pyridine (2.56 mL, 31.7 mmol, 5.00 eq) and phenyl chloroformate (1.59 mL, 12.7 mmol, 2.00 eq) in portions at 0° C. The reaction was stirred at 25° C. for 1 h. The mixture was concentrated under reduced pressure to give a residue, which was diluted with water (50.0 mL) and exacted with ethyl acetate (3×30.0 mL). The combined organic layers were washed with water, dried over anhydrous sodium sulfate, filtered, and concentrated under reduced pressure to give a residue. The residue was purified by reversed phase preparative HPLC. The desired fraction was collected, and acetonitrile was removed under reduced pressure. The residual aqueous solution was exacted with ethyl acetate (3×50.0 mL). The combined organic layers were washed with brine, dried over anhydrous sodium sulfate, filtered, and concentrated to afford phenyl(5-chloro-2-methoxyphenyl)carbamate.

Compound 215: General procedure A with variant i) was used for the preparation from compound VIII employing phenyl(6-(piperidin-1-yl)pyridin-3-yl)carbamate. ¹H NMR (400 MHz, DMSO-d₆) δ=10.99 (s, 1H), 10.12 (br s, 1H), 8.19 (br s, 1H), 7.95-7.89 (m, 1H), 7.79 (s, 1H), 7.70-7.62 (m, 2H), 7.41 (br d, J=9.3 Hz, 1H), 5.29 (s, 2H), 5.12 (dd, J=5.1, 13.3 Hz, 1H), 4.52-4.44 (m, 1H), 4.39-4.31 (m, 1H), 3.64 (br s, 4H), 2.91 (ddd, J=5.4, 13.6, 17.5 Hz, 1H), 2.63-2.58 (m, 1H), 2.45-2.35 (m, 1H), 2.06-1.96 (m, 1H), 1.63 (br s, 6H). MS (ESI) m/z 478.1 [M+H]⁺

Step 1: To a solution of 2-fluoro-5-nitro-pyridine (5.00 g, 35.2 mmol, 1.00 eq) in acetonitrile (50.0 mL) were added potassium carbonate (9.73 g, 70.4 mmol, 2.00 eq) and piperidine (4.17 mL, 42.2 mmol, 1.20 eq). The reaction was stirred at 25° C. for 1 h. The mixture was filtered and concentrated under reduced pressure to afford 5-nitro-2-(1-piperidyl)pyridine.

Step 2: To a solution of 5-nitro-2-(piperidin-1-yl)pyridine (5.20 g, 25.1 mmol, 1.00 eq) and ammonium chloride (6.71 g, 125 mmol, 5.00 eq) in methanol (40.0 mL) and water (10.0 mL) was added iron powder (7.01 g, 126 mmol, 5.00 eq) in portions. The reaction was stirred at 80° C. for 2 h. The mixture was filtered, and the filtrate was concentrated under reduced pressure to give a residue. The residue was diluted with water (150 mL) and extracted with ethyl acetate (3×60.0 mL). The combined organic layers were washed with brine (60.0 mL), dried over sodium sulfate, filtered, and concentrated under reduced pressure to give a residue. The residue was purified by reversed phase preparative HPLC. The desired fraction was collected, diluted with saturated sodium bicarbonate (150 mL), and extracted with ethyl acetate (3×60.0 mL). The combined organic layers were washed with brine (60.0 mL), dried over sodium sulfate, filtered, and concentrated under reduced pressure to afford 6-(piperidin-1-yl)pyridin-3-amine.

Step 3: To a solution of 6-(piperidin-1-yl)pyridin-3-amine (300 mg, 1.69 mmol, 1.00 eq) and pyridine (0.41 mL, 5.08 mmol, 3.00 eq) in acetonitrile (3.00 mL) was added phenyl chloroformate (0.32 mL, 2.54 mmol, 1.50 eq) dropwise at 0° C. The reaction was stirred at 25° C. for 12 h. The mixture was diluted with water (50.0 mL) and extracted with ethyl acetate (3×40.0 mL). The combined organic layers were washed with brine (20.0 mL), dried over sodium sulfate, filtered, and concentrated under reduced pressure to give a residue. The residue was purified by reversed phase preparative HPLC. The desired fraction was collected, diluted with saturated sodium bicarbonate (30 mL), and extracted with ethyl acetate (3×40.0 mL). The combined organic layers were washed with brine (20.0 mL), dried over sodium sulfate, filtered, and concentrated to afford phenyl (6-(piperidin-1-yl)pyridin-3-yl)carbamate.

### Compound 216:

Step 1: To a solution of tert-butyl 3-(hydroxymethyl)pyrrolidine-1-carboxylate (3.00 g, 14.9 mmol, 1.00 eq) and triethylamine (5.19 mL, 37.3 mmol, 2.50 eq) in dichloromethane (30.0 mL) at 0° C. was added methylsulfamoyl chloride (1.50 mL, 19.4 mmol, 1.30 eq) dropwise over 2 min under nitrogen atmosphere. The reaction was then stirred at 25° C. for 2 h. The mixture was diluted with ethyl acetate (100 mL) and water (150 mL). The organic layer was separated, and the aqueous phase was extracted with ethyl acetate (3×100 mL). The combined organic layers were dried over sodium sulfate, filtered, and concentrated under reduced pressure to afford tert-butyl 3-(((methylsulfonyl)oxy)methyl)pyrrolidine-1-carboxylate.

Step 2: A solution of tert-butyl 3-(((methylsulfonyl)oxy)methyl) pyrrolidine-1-carboxylate (3.00 g, 10.8 mmol, 1.00 eq), 3-chloro-5-nitrophenol (2.05 g, 11.8 mmol, 1.10 eq) and caesium carbonate (10.5 g, 32.2 mmol, 3.00 eq) in dimethylformamide (30.0 mL) was stirred at 80° C. for 12 h. The mixture was diluted with ethyl acetate (100 mL) and water (100 mL). The organic layer was separated, and the aqueous phase was extracted with ethyl acetate (3×80.0 mL). The combined organic layers were dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl)pyrrolidine-1-carboxylate.

Step 3: A solution of tert-butyl 3-((3-chloro-5-nitrophenoxy)methyl)pyrrolidine-1-carboxylate (2.00 g, 5.61 mmol, 1.00 eq), iron powder (2.19 g, 39.2 mmol, 7.00 eq) and ammonium chloride (2.10 g, 39.2 mmol, 7.00 eq) in methanol (30.0 mL) and water (30.0 mL) was stirred at 80° C. for 2 h. The mixture was filtered over Celite, and the filtrate was concentrated under reduced pressure to afford tert-butyl 3-((3-amino-5-chlorophenoxy) methyl)pyrrolidine-1-carboxylate.

Step 4: A solution of phenyl chloroformate (0.11 mL, 918 μmol, 1.50 eq), tert-butyl 3-((3-amino-5-chlorophenoxy)methyl)pyrrolidine-1-carboxylate (200 mg, 612 μmol, 1.00 eq) and pyridine (0.15 mL, 1.84 mmol, 3.00 eq) in acetonitrile (2.00 mL) was stirred at 25° C. for 12 h. The mixture was diluted with ethyl acetate (80.0 mL) and water (80.0 mL). The organic layer was separated, and the aqueous phase was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-((phenoxycarbonyl)amino)phenoxy) methyl)pyrrolidine-1-carboxylate.

Step 5: A solution of 3-(6-(hydroxymethyl)-1-oxoisoindolin-2-yl)piperidine-2,6-dione VIII (80.0 mg, 292 μmol, 1.00 eq), tert-butyl 3-((3-chloro-5-((phenoxycarbonyl)amino)phenoxy)methyl) pyrrolidine-1-carboxylate (156 mg, 350 μmol, 1.20 eq) and sodium hydride (60% dispersion in mineral oil) (23.3 mg, 583 μmol, 2.00 eq) in dimethylformamide (2.00 mL) was stirred at 0° C. for 4 h. The mixture was quenched by addition by hydrochloric acid (1 M, 5.00 mL) and the filtrate was concentrated under reduced pressure to afford tert-butyl 3-((3-chloro-5-((((2-(2,6-dioxopiperidin-3-yl)-3-oxoisoindolin-5-yl)methoxy)carbonyl)amino)phenoxy)methyl)pyrrolidine-1-carboxylate.

Step 6: A solution of tert-butyl 3-((3-chloro-5-((((2-(2,6-dioxopiperidin-3-yl)-3-oxoisoindolin-5-yl)methoxy)carbonyl)amino)phenoxy)methyl)pyrrolidine-1-carboxylate (200 mg, 319 μmol, 1.00 eq) and hydrochloric acid (12 M, 26.6 μL, 1.00 eq) in water (2.00 mL) was stirred at 25° C. for 12 h. The mixture was diluted with acetonitrile (2.00 mL) and filtered. The filtrate was purified by a standard method to afford Compound 216. ¹H NMR (400 MHz, DMSO-d₆) δ=10.99 (s, 1H), 10.04 (s, 1H), 9.12 (br d, J=2.7 Hz, 2H), 7.79 (s, 1H), 7.71-7.61 (m, 2H), 7.15-7.09 (m, 2H), 6.70 (t, J=2.0 Hz, 1H), 5.28 (s, 2H), 5.16-5.08 (m, 1H), 4.52-4.43 (m, 1H), 4.38-4.31 (m, 1H), 4.03-3.93 (m, 2H), 3.30-3.10 (m, 3H), 3.05-2.84 (m, 2H), 2.78-2.65 (m, 1H), 2.60 (br d, J=16.8 Hz, 1H), 2.47-2.35 (m, 1H), 2.13-1.96 (m, 2H), 1.80-1.68 (m, 1H). MS (ESI) m/z 527.3 [M+H]⁺

### Compound 217:

Step 1: To a solution of 2-(2,6-dioxo-1-((2-(trimethylsilyl)ethoxy)methyl)piperidin-3-yl)-3-oxoisoindoline-5-Carbaldehyde V (750 mg, 1.86 mmol, 1.00 eq) in dichloromethane (10.0 mL) was added methylmagnesium bromide 3.00 M in diethyl ether (3.00 M, 0.75 mL, 1.20 eq) dropwise at −78° C. The reaction was stirred at −78° C. for 2 h. The reaction was quenched with ammonium chloride (50.0 mL) to reach pH=7, and the mixture was extracted with ethyl acetate (3×30.0 mL). The combined organic layers were washed with brine (20.0 mL), dried over anhydrous sodium sulfate, filtered, and concentrated under reduces pressure to give a residue. The residue was purified by silica gel column chromatography (petroleum ether/ethyl acetate=10/1 to 2/3) to afford 3-(6-(1-hydroxyethyl)-1-oxoisoindolin-2-yl)-1-((2-(trimethylsilyl) ethoxy)methyl)piperidine-2,6-dione.

Step 2: A solution of 3-(6-(1-hydroxyethyl)-1-oxoisoindolin-2-yl)-1-((2-(trimethylsilyl)ethoxy) methyl)piperidine-2,6-dione (600 mg, 1.43 mmol, 1.00 eq) in hydrochloric acid/dioxane (6 M, 6.67 mL, 27.9 eq) was stirred at 50° C. for 1 h. The mixture was concentrated under reduced pressure to afford 3-(6-(1-hydroxyethyl)-1-oxoisoindolin-2-yl)-1-(hydroxymethyl)piperidine-2,6-dione.

Step 3: To a solution of 3-(6-(1-hydroxyethyl)-1-oxoisoindolin-2-yl)-1-(hydroxymethyl) piperidine-2,6-dione (450 mg, 1.41 mmol, 1.00 eq) in acetonitrile (10.0 mL) was added ammonium hydroxide 30% (0.20 mL, 1.56 mmol, 1.10 eq) dropwise. The reaction was stirred at 25° C. for 0.5 h. The reaction was quenched with 1 M HCl to reach pH=3 and concentrated under reduced pressure to give a residue. The residue was purified by reversed phase preparative HPLC to afford 3-(6-(1-hydroxyethyl)-1-oxoisoindolin-2-yl)piperidine-2,6-dione.

Step 4: To a solution of 3-(6-(1-hydroxyethyl)-1-oxoisoindolin-2-yl)piperidine-2,6-dione (85.0 mg, 295 μmol, 1.00 eq) and phenyl (3-chloro-4-methylphenyl)carbamate (described in example 1) (84.9 mg, 324 μmol, 1.10 eq) in dimethylformamide (3.00 mL) was added sodium hydride (60% dispersion in mineral oil) (23.6 mg, 590 μmol, 2.00 eq) in portions at 0° C. The reaction was stirred at 0° C. for 0.5 h. The reaction was quenched with 1 M hydrochloric acid (2.00 mL), filtered, and concentrated to give a residue. The residue was purified by a standard method to afford Compound 217. ¹H NMR (400 MHz, DMSO-d₆) δ=11.00 (br s, 1H), 9.93 (br s, 1H), 7.77 (s, 1H), 7.71-7.60 (m, 2H), 7.58 (d, J=1.6 Hz, 1H), 7.35-7.18 (m, 2H), 5.91 (q, J=6.4 Hz, 1H), 5.13 (dd, J=5.0, 13.3 Hz, 1H), 4.56-4.26 (m, 2H), 2.98-2.86 (m, 1H), 2.65-2.56 (m, 1H), 2.45-2.35 (m, 1H), 2.24 (s, 3H), 2.06-1.96 (m, 1H), 1.58 (d, J=6.6 Hz, 3H). MS (ESI) m/z 456.1 [M+H]⁺

Compound 218: General procedure A with variant i) was used for the preparation from compound VIII employing phenyl (2,3-dihydrobenzofuran-6-yl)carbamate.

¹H NMR (400 MHz, DMSO-d₆) δ=10.99 (s, 1H), 9.72 (brs, 1H), 7.79 (s, 1H), 7.69-7.61 (m, 2H), 7.09 (d, J=8.0 Hz, 1H), 6.98 (s, 1H), 6.88 (br d, J=7.9 Hz, 1H), 5.25 (s, 2H), 5.13 (dd, J=5.1, 13.2 Hz, 1H), 4.54-4.43 (m, 3H), 4.38-4.30 (m, 1H), 3.08 (t, J=8.6 Hz, 2H), 2.98-2.85 (m, 1H), 2.63-2.57 (m, 1H), 2.43-2.33 (m, 1H), 2.06-1.96 (m, 1H). MS (ESI) m/z 436.2 [M+H]⁺

To a solution of 2,3-dihydrobenzofuran-6-amine (200 mg, 1.48 mmol, 1.00 eq) in acetonitrile (8.00 mL) were added pyridine (0.60 mL, 7.40 mmol, 5.00 eq) and phenyl chloroformate (0.37 mL, 2.96 mmol, 2.00 eq) at 0° C. in portions. The reaction was stirred at 25° C. for 1 h. The mixture was concentrated and the obtained residue was purified by standard methods to afford phenyl (2,3-dihydrobenzofuran-6-yl)carbamate.

Compound 219: General procedure A with variant i) was used for the preparation from compound VIII employing phenyl (3-chloro-5-(difluoromethoxy)phenyl)carbamate.

¹H NMR (400 MHz, DMSO-d₆) 5=10.99 (s, 1H), 10.23 (s, 1H), 7.79 (s, 1H), 7.72-7.61 (m, 2H), 7.42 (t, J=1.8 Hz, 1H), 7.31 (s, 1H), 7.06 (t, J=44 Hz, 1H), 6.94 (t, J=2.0 Hz, 1H), 5.29 (s, 2H), 5.12 (dd, J=5.1, 13.3 Hz, 1H), 4.51-4.43 (m, 1H), 4.38-4.30 (m, 1H), 2.97-2.85 (m, 1H), 2.60 (br d, J=17.5 Hz, 1H), 2.40 (dd, J=4.4, 13.1 Hz, 1H), 2.06-1.96 (m, 1H). MS (ESI) m/z 494.1 [M+H]⁺

Step 1: To a solution of 3-chloro-5-nitro-phenol (1.50 g, 8.64 mmol, 1.00 eq) and sodium 2-chloro-2,2-difluoroacetate (5.27 g, 34.6 mmol, 4.00 eq) in dimethylformamide (17.0 mL) and water (2.00 mL) was added potassium carbonate (2.39 g, 17.3 mmol, 2.00 eq). The reaction was stirred at 100° C. for 12 h. The mixture was filtered and concentrated under reduced pressure to give a residue. The residue was diluted with water (100 mL) and extracted with ethyl acetate (3×20.0 mL). The combined organic layers were washed with saturated sodium carbonate solution (50.0 mL) and brine (20.0 mL), dried over anhydrous sodium sulfate, filtered, and concentrated under reduced pressure to afford 1-chloro-3-(difluoromethoxy)-5-nitrobenzene.

Step 2: To a solution of 1-chloro-3-(difluoromethoxy)-5-nitro-benzene (1.70 g, 7.60 mmol, 1.00 eq) in methanol (10.0 mL) and water (10.0 mL) were added iron powder (2.12 g, 38.0 mmol, 5.00 eq) and ammonium chloride (2.03 g, 38.0 mmol, 5.00 eq). The reaction was stirred at 80° C. for 2 h. The mixture was concentrated under reduced pressure to give a residue. The residue was diluted with saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-5-(difluoromethoxy)aniline.

Step 3: To a solution 3-chloro-5-(difluoromethoxy)aniline (400 mg, 2.07 mmol, 1.00 eq) in acetonitrile (10.0 mL) were added pyridine (0.83 mL, 10.3 mmol, 5.00 eq) and phenyl chloroformate (0.39 mL, 3.10 mmol, 1.50 eq). The reaction was stirred at 25° C. for 1 h. The mixture was concentrated and the obtained residue was purified by standard methods to afford phenyl (3-chloro-5-(difluoromethoxy)phenyl)carbamate.

### Compound 220:

Step 1: To a solution of 3-(6-(hydroxymethyl)-1-oxoisoindolin-2-yl)piperidine-2,6-dione (120 mg, 437 μmol, 1.00 eq) and tert-butyl 3-(3-chloro-5-((phenoxycarbonyl)amino) phenoxy)pyrrolidine-1-carboxylate (284 mg, 656 μmol, 1.50 eq) in dimethylformamide (2.00 mL) was added sodium hydride (60% dispersion in mineral oil) (35.0 mg, 875 μmol, 2.00 eq). The reaction was stirred at 25° C. for 1 h.

Step 2: A solution of tert-butyl 3-(3-chloro-5-((((2-(2,6-dioxopiperidin-3-yl)-3-oxoisoindolin-5-yl)methoxy)carbonyl)amino)phenoxy)pyrrolidine-1-carboxylate (250 mg, 407 μmol, 1.00 eq) in hydrochloric acid (4 M, 5.00 mL, 49.0 eq) was stirred at 25° C. for 12 h. The mixture was concentrated under reduced pressure to give a residue. The residue was purified by a standard method to afford Compound 220. ¹H NMR (400 MHz, DMSO-d₆) δ=11.00 (s, 1H), 10.07 (s, 1H), 9.32-9.13 (m, 2H), 7.80 (s, 1H), 7.75-7.60 (m, 2H), 7.23-7.09 (m, 2H), 6.75 (t, J=2.0 Hz, 1H), 5.29 (s, 2H), 5.18-5.08 (m, 2H), 4.56-4.41 (m, 1H), 4.41-4.31 (m, 1H), 3.32-3.21 (m, 2H), 3.00-2.87 (m, 1H), 2.63 (br d, J=2.8 Hz, 2H), 2.43 (dt, J=4.5, 13.1 Hz, 2H), 2.25-2.10 (m, 2H), 2.06-1.97 (m, 1H). MS (ESI) m/z 513.0 [M+H]⁺

### Preparation of tert-butyl 3-(3-chloro-5-((phenoxycarbonyl)amino) phenoxy)pyrrolidine-1-carboxylate

Step 1: To a solution of 3-chloro-5-nitro-phenol (5.00 g, 28.8 mmol, 1.00 eq), tert-butyl 3-hydroxypyrrolidine-1-carboxylate (5.93 g, 31.7 mmol, 1.10 eq) and triphenylphosphine (8.31 g, 31.7 mmol, 1.10 eq) in tetrahydrofuran (2.00 mL) was added diisopropyl azodiformate (6.16 mL, 31.7 mmol, 1.10 eq) at 0° C. The reaction was stirred at 25° C. for 12 h. The mixture was concentrated under reduced pressure to give a residue. The residue was purified by reversed phase preparative HPLC to afford tert-butyl 3-(3-chloro-5-nitrophenoxy)pyrrolidine-1-carboxylate.

Step 2: A mixture of tert-butyl 3-(3-chloro-5-nitro-phenoxy)pyrrolidine-1-carboxylate (3.00 g, 8.75 mmol, 100 eq), iron powder (1.47 g, 26.2 mmol, 3.00 eq) and ammonium chloride (2.34 g, 43.8 mmol, 5.00 eq) in methanol (20.0 mL) and water (10.0 mL) was stirred at 80° C. for 2 h. The mixture was filtered, and the filtrate was concentrated under reduced pressure to give a residue. It was added to water (80.0 mL) and saturated sodium bicarbonate (40.0 mL) and stirred for 10 min. The aqueous layer was extracted with ethyl acetate (3×60.0 mL). The combined organic layers were washed with brine (100 mL), dried over anhydrous sodium sulfate, filtered, and concentrated under reduced pressure to afford tert-butyl 3-(3-amino-5-chlorophenoxy)pyrrolidine-1-carboxylate.

Compound 221: General procedure A with variant i) was used for the preparation from compound VIII employing phenyl (3-(8-oxa-3-azabicyclo[3.2.1]octan-3-ylmethyl)-5-chloro-4-methylphenyl)carbamate. ¹H NMR (400 MHz, DMSO-d₆) δ=11.05-10.93 (m, 1H), 9.95-9.84 (m, 1H), 7.79 (s, 1H), 7.71-7.61 (m, 2H), 7.51 (s, 1H), 7.33 (d, J=1.7 Hz, 1H), 5.27 (s, 2H), 5.12 (dd, J=5.2, 13.4 Hz, 1H), 4.51-4.42 (m, 1H), 4.39-4.29 (m, 1H), 4.21-4.15 (m, 2H), 2.95-2.86 (m, 1H), 2.60 (br dd, J=1.7, 16.8 Hz, 1H), 2.45 (br d, J=10.3 Hz, 4H), 2.41-2.36 (m, 1H), 2.29-2.25 (m, 3H), 2.19 (br d, J=9.8 Hz, 2H), 2.04-1.98 (m, 1H), 1.88-1.79 (m, 2H), 1.68 (br dd, J=4.1, 7.0 Hz, 2H). MS (ESI) m/z 567.2 [M+H]⁺

Step 1: To a solution of (3-chloro-2-methyl-5-nitro-phenyl)methanol (2.00 g, 9.92 mmol, 1.00 eq) in dichloromethane (30.0 mL) was added thionyl chloride (3.60 mL, 49.6 mmol, 5.00 eq) at 25° C. The reaction was stirred at 25° C. for 12 h. The reaction was quenched by addition of ice water (20.0 m L) at 0° C., and the aqueous layer was extracted with ethyl acetate (3×20.0 mL). The combined organic layers were washed with brine (30.0 mL), dried over anhydrous sodium sulfate, filtered, and concentrated under reduced pressure to afford 1-chloro-3-(chloromethyl)-2-methyl-5-nitrobenzene.

Step 2: To a solution of 1-chloro-3-(chloromethyl)-2-methyl-5-nitro-benzene (1.40 g, 6.36 mmol, 1.00 eq), 8-oxa-3-azabicyclo[3.2.1]octane (1.14 g, 7.63 mmol, 1.20 eq, HCl) and potassium carbonate (2.64 g, 19.1 mmol, 3.00 eq) in acetonitrile (20.0 mL) was added potassium iodide (106 mg, 0.64 mmol, 0.10 eq) at 25° C. The reaction was stirred at 80° C. for 12 h. The mixture was filtered and concentrated under reduced pressure to give a residue. The residue was purified by silica gel column chromatography (petroleum ether/ethyl acetate=10/1 to 2/1) to afford 3-(3-chloro-2-methyl-5-nitrobenzyl)-8-oxa-3-azabicyclo[3.2.1]octane.

Step 3: To a solution of 3-(3-chloro-2-methyl-5-nitrobenzyl)-8-oxa-3-azabicyclo[3.2.1]octane (1.00 g, 3.37 mmol, 1.00 eq) in methanol (20.0 mL) were added iron powder (941 mg, 16.9 mmol, 5.00 eq), ammonium chloride (901 mg, 16.9 mmol, 5.00 eq) and water (5.00 mL) at 25° C. The reaction was stirred at 80° C. for 2 h. The mixture was concentrated under reduced pressure to give a residue. Sodium bicarbonate (20.0 mL) was added, and the aqueous layer was extracted with ethyl acetate (3×20.0 mL). The combined organic layers were washed with brine (20.0 mL), dried over anhydrous sodium sulfate, filtered, and concentrated under reduced pressure to afford 3-(8-oxa-3-azabicyclo[3.2.1]octan-3-ylmethyl)-5-chloro-4-methylaniline.

Step 4: To a solution of 3-(8-oxa-3-azabicyclo[3.2.1]octan-3-ylmethyl)-5-chloro-4-methylaniline (200 mg, 749 μmol, 1.00 eq) in acetonitrile (5.00 mL) were added pyridine (0.30 mL, 3.75 mmol, 5.00 eq) and phenyl chloroformate (0.14 mL, 1.12 mmol, 1.50 eq). The reaction was stirred at 25° C. for 1 h. The mixture was concentrated and the obtained residue was purified by standard methods to afford phenyl (3-(8-oxa-3-azabicyclo[3.2.1]octan-3-ylmethyl)-5-chloro-4-methylphenyl)carbamate.

Compound 222: General procedure A with variant ii) was used for the preparation from compound VIII employing cyclopropylmethanamine. ¹H NMR (400 MHz, DMSO-d₆) δ=10.99 (s, 1H), 7.71 (s, 1H), 7.60 (s, 2H), 7.40 (brt, J=5.6 Hz, 1H), 5.16-5.06 (m, 3H), 4.49-4.42 (m, 1H), 4.36-4.29 (m, 1H), 2.96-2.85 (m, 3H), 2.65-2.56 (m, 1H), 2.43-2.37 (m, 1H), 2.06-1.96 (m, 1H), 0.96-0.85 (m, 1H), 0.43-0.35 (m, 2H), 0.19-0.09 (m, 2H). MS (ESI) m/z 372.1 [M+H]⁺

Compound 223: General procedure A with variant ii) was used for the preparation from compound VIII employing piperidine. ¹H NMR (400 MHz, DMSO-d₆) δ=11.06-10.92 (m, 1H), 7.69 (s, 1H), 7.64-7.54 (m, 2H), 5.17 (s, 2H), 5.11 (dd, J=5.1, 13.3 Hz, 1H), 4.51-4.41 (m, 1H), 4.38-4.28 (m, 1H), 3.37 (br s, 4H), 2.97-2.85 (m, 1H), 2.60 (br d, J=17.6 Hz, 1H), 2.40 (br dd, J=4.3, 13.0 Hz, 1H), 2.06-1.95 (m, 1H), 1.59-1.50 (m, 2H), 1.48-1.39 (m, 4H). MS (ESI) m/z 386.1 [M+H]⁺

Compound 224: General procedure A with variant i) was used for the preparation from compound VIII employing phenyl (3-(difluoromethoxy)-4-methylphenyl)carbamate. ¹H NMR (400 MHz, DMSO-d₆) δ=10.99 (br s, 1H), 9.92 (br s, 1H), 7.79 (s, 1H), 7.70-7.61 (m, 2H), 7.41 (s, 1H), 7.19 (s, 2H), 7.09 (t, J=74 Hz, 1H), 5.27 (s, 2H), 5.12 (dd, J=5.1, 13.2 Hz, 1H), 4.52-4.43 (m, 1H), 4.38-4.30 (m, 1H), 2.96-2.85 (m, 1H), 2.63-2.57 (m, 1H), 2.40 (br dd, J=4.3, 13.1 Hz, 1H), 2.15 (s, 3H), 2.04-1.96 (m, 1H). MS (ESI) m/z 474.1 [M+H]⁺

Step 1: To a solution of 2-methyl-5-nitrophenol (5.00 g, 32.7 mmol, 1.00 eq) and sodium 2-chloro-2,2-difluoroacetate (12.4 g, 81.6 mmol, 2.50 eq) in dimethylformamide (50.0 mL) was added caesium carbonate (21.3 g, 65.3 mmol, 2.00 eq) in portions. The reaction was stirred at 100° C. for 2 h. The mixture was diluted with water (800 mL) and extracted with ethyl acetate (3×100 mL). The combined organic layers were washed with brine (80.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-(difluoromethoxy)-1-methyl-4-nitrobenzene.

Step 2: To a solution of 2-(difluoromethoxy)-1-methyl-4-nitrobenzene (4.85 g, 23.8 mmol, 1.00 eq) and ammonium chloride (6.39 g, 119 mmol, 5.00 eq) in methanol (40.0 mL) and water (40.0 mL) was added iron powder (4.00 g, 71.6 mmol, 3.00 eq) in portions. The reaction was stirred at 80° C. for 2 h. The mixture was filtered, and the filtrate was concentrated under reduced pressure to give a residue. Water (100 mL) was added, and the mixture was extracted with ethyl acetate (3×50.0 mL). The combined organic layers were washed with brine (20 mL), dried over anhydrous sodium sulfate, filtered, and concentrated to afford 3-(difluoromethoxy)-4-methylaniline.

Step 3: To a solution of 3-(difluoromethoxy)-4-methylaniline (1.00 g, 5.78 mmol, 1.00 eq) and pyridine (1.40 mL, 17.3 mmol, 3.00 eq) in acetonitrile (10.0 mL) was added phenyl chloroformate (1.09 mL, 8.66 mmol, 1.50 eq) dropwise. The reaction was stirred at 25° C. for 12 h. The mixture was diluted with water (150 mL) and extracted with ethyl acetate (3×50.0 mL). The combined organic layers were washed with brine (20.0 mL), dried over anhydrous sodium sulfate, filtered, and concentrated and the obtained residue was purified by standard methods to afford phenyl (3-(difluoromethoxy)-4-methylphenyl)carbamate.

Compound 225: General procedure A with variant i) was used for the preparation from compound VIII employing phenyl (3-(difluoromethoxy)-4-methyl-5-(morpholinomethyl)phenyl)carbamate. ¹H NMR (400 MHz, DMSO-d₆) δ=10.99 (s, 1H), 9.89 (s, 1H), 8.21 (s, 1H), 7.78 (s, 1H), 7.70-7.60 (m, 2H), 7.35 (br s, 1H), 7.26 (d, J=1.8 Hz, 1H), 7.25-6.85 (m, 1H), 5.27 (s, 2H), 5.16-5.08 (m, 1H), 4.51-4.28 (m, 2H), 3.55 (brt, J=4.3 Hz, 4H), 3.39 (s, 2H), 2.98-2.84 (m, 1H), 2.65-2.56 (m, 1H), 2.45-2.38 (m, 1H), 2.35 (br s, 4H), 2.14 (s, 3H), 2.06-1.96 (m, 1H). MS (ESI) m/z 573.4 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and the resulting precipitate was collected by filtration. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.100 eq), and quinolin-8-ol (0.56 mL, 3.26 mmol, 0.200 eq) in water (3.00 mL) and dimethylsulfoxide (3.00 mL) was added potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to afford a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: A solution of (3-hydroxy-2-methyl-5-nitro-phenyl)-morpholino-methanone (2.00 g, 7.51 mmol, 1.00 eq), potassium carbonate (2.08 g, 15.0 mmol, 2.00 eq) and sodium 2-chloro-2,2-difluoroacetate (4.58 g, 30.0 mmol, 4.00 eq) in dimethylformamide (24.0 mL) and water (3.00 mL) was stirred at 100° C. for 12 h. The mixture was diluted with water (150 mL) and extracted with ethyl acetate (3×100 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=3/1) to afford (3-(difluoromethoxy)-2-methyl-5-nitrophenyl)(morpholino)methanone.

Step 5: To a solution of (3-(difluoromethoxy)-2-methyl-5-nitrophenyl)(morpholino)methanone (1.70 g, 5.38 mmol, 1.00 eq) in tetrahydrofuran (3.00 mL) was added borane dimethyl sulfide complex (10 M, 1.08 mL, 2.00 eq) dropwise at 0° C. under nitrogen atmosphere. The reaction was stirred at 60° C. for 4 h. The reaction was quenched by addition with methanol (5.00 mL) and the solvents were removed under reduced pressure to afford 4-(3-(difluoromethoxy)-2-methyl-5-nitrobenzyl)morpholine.

Step 6: A solution of 4-(3-(difluoromethoxy)-2-methyl-5-nitrobenzyl)morpholine (1.50 g, 4.96 mmol, 1.00 eq), iron powder (1.94 g, 34.7 mmol, 7.00 eq) and ammonium chloride (1.86 g, 34.7 mmol, 7.00 eq) in methanol (5.00 mL) and water (5.00 mL) was stirred at 80° C. for 2 h. The mixture was filtered, and the filtrate was concentrated under reduced pressure to afford 3-(difluoromethoxy)-4-methyl-5-(morpholinomethyl)aniline.

Step 7: To a solution of 3-(difluoromethoxy)-4-methyl-5-(morpholinomethyl)aniline (500 mg, 1.84 mmol, 1.00 eq) in acetonitrile (5.00 mL) were added pyridine (0.45 mL, 5.51 mmol, 3.00 eq) and phenyl chloroformate (0.28 mL, 2.20 mmol, 1.20 eq). The reaction was stirred at 25° C. for 2 h. The mixture was diluted with ethyl acetate (80.0 mL) and water (80.0 mL). The organic layer was separated, and the aqueous phase was extracted with ethyl acetate (3×50.0 mL). The combined organic layers were dried over sodium sulfate, filtered, and concentrated and the obtained residue was purified by standard methods to afford phenyl (3-(difluoromethoxy)-4-methyl-5-(morpholinomethyl)phenyl) carbamate.

Compound 226: General procedure A with variant ii) was used for the preparation from compound VIII employing pyrrolidine. ¹H NMR (400 MHz, DMSO-d₆) δ=10.99 (s, 1H), 7.70 (s, 1H), 7.65-7.58 (m, 2H), 5.17 (s, 2H), 5.12 (dd, J=5.1, 13.3 Hz, 1H), 4.51-4.41 (m, 1H), 4.37-4.27 (m, 1H), 3.31-3.25 (m, 4H), 2.98-2.86 (m, 1H), 2.60 (br d, J=17.5 Hz, 1H), 2.43-2.32 (m, 1H), 2.05-1.97 (m, 1H), 1.86-1.76 (m, 4H). MS (ESI) m/z 372.2 [M+H]⁺

Compound 227: General procedure A with variant ii) was used for the preparation from compound VIII employing 3-methylbutan-1-amine. ¹H NMR (400 MHz, DMSO-d₆) δ=10.99 (s, 1H), 7.70 (s, 1H), 7.66-7.55 (m, 2H), 7.28 (br t, J=5.4 Hz, 1H), 5.22-5.06 (m, 3H), 4.52-4.42 (m, 1H), 4.38-4.28 (m, 1H), 3.06-2.98 (m, 2H), 2.97-2.86 (m, 1H), 2.64-2.57 (m, 1H), 2.44-2.36 (m, 1H), 2.06-1.97 (m, 1H), 1.63-1.50 (m, 1H), 1.30 (q, J=6.9 Hz, 2H), 0.86 (d, J=6.6 Hz, 6H). MS (ESI) m/z 388.2 [M+H]⁺

Compound 228: General procedure A with variant ii) was used for the preparation from compound VIII employing cyclohexanamine. ¹H NMR (400 MHz, DMSO-d₆) δ=11.00 (s, 1H), 7.72 (s, 1H), 7.61 (s, 2H), 7.25 (br d, J=7.9 Hz, 1H), 5.18-5.08 (m, 3H), 4.50-4.42 (m, 1H), 4.37-4.29 (m, 1H), 3.30-3.22 (m, 1H), 2.98-2.86 (m, 1H), 2.61 (br dd, J=2.1, 15.6 Hz, 1H), 2.44-2.34 (m, 1H), 2.06-1.95 (m, 1H), 1.76 (br d, J=11.7 Hz, 2H), 1.71-1.61 (m, 2H), 1.54 (br d, J=12.8 Hz, 1H), 1.31-1.07 (m, 5H). MS (ESI) m/z 400.2 [M+H]⁺

Compound 229: General procedure A with variant ii) was used for the preparation from compound VIII employing cyclopentanamine. ¹H NMR (400 MHz, DMSO-d₆) δ=10.98 (br s, 1H), 7.71 (s, 1H), 7.60 (s, 2H), 7.31 (br d, J=7.1 Hz, 1H), 5.19-5.07 (m, 3H), 4.50-4.41 (m, 1H), 4.37-4.28 (m, 1H), 3.85-3.74 (m, 1H), 2.98-2.85 (m, 1H), 2.65-2.57 (m, 1H), 2.43-2.34 (m, 1H), 2.05-1.95 (m, 1H), 1.83-1.71 (m, 2H), 1.65-1.54 (m, 2H), 1.52-1.35 (m, 4H). MS (ESI) m/z 386.1 [M+H]⁺

Compound 230: General procedure A with variant ii) was used for the preparation from compound VIII employing 3-phenoxypyrrolidine. ¹H NMR (400 MHz, DMSO-d₆) δ=11.00 (s, 1H), 7.77-7.68 (m, 1H), 7.68-7.56 (m, 2H), 7.37-7.20 (m, 2H), 7.05-6.84 (m, 3H), 5.30-5.08 (m, 3H), 5.07-4.98 (m, 1H), 4.54-4.40 (m, 1H), 4.38-4.27 (m, 1H), 3.72-3.57 (m, 1H), 3.55-3.41 (m, 3H), 2.97-2.87 (m, 1H), 2.65-2.58 (m, 1H), 2.41 (br dd, J=4.5, 12.8 Hz, 1H), 2.19-1.99 (m, 3H). MS (ESI) m/z 464.1 [M+H]⁺

Step 1: To a solution of tert-butyl 3-hydroxypyrrolidine-1-carboxylate (3.00 g, 16.0 mmol, 1.00 eq) and triethylamine (5.58 mL, 40.0 mmol, 2.50 eq) in dichloromethane (30.0 mL) was added methanesulfonyl chloride (1.74 mL, 22.4 mmol, 1.40 eq) dropwise at 0° C. The reaction was stirred at 25° C. for 2 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford tert-butyl 3-((methylsulfonyl)oxy)pyrrolidine-1-carboxylate.

Step 2: To a solution of tert-butyl 3-((methylsulfonyl)oxy)pyrrolidine-1-carboxylate (1.00 g, 3.77 mmol, 1.00 eq) and phenol (0.40 mL, 4.52 mmol, 1.20 eq) in dimethylformamide (10.0 mL) was added caesium carbonate (3.68 g, 11.3 mmol, 3.00 eq) in one portion. The reaction was stirred at 80° C. for 12 h. The mixture was diluted with water (50.0 mL) and extracted with ethyl acetate (3×60.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=20/1 to 5/1) and then by reversed phase preparative HPLC to afford tert-butyl 3-phenoxypyrrolidine-1-carboxylate.

Step 3: A solution of tert-butyl 3-phenoxypyrrolidine-1-carboxylate (460 mg, 1.75 mmol, 1.00 eq) in 4 M hydrochloric acid/dioxane (4.00 mL) was stirred at 25° C. for 1 h and concentrated to afford 3-phenoxypyrrolidine.

Compound 231: General procedure A with variant i) was used for the preparation from compound VIII employing phenyl (3-(difluoromethoxy)-5-fluorophenyl) carbamate. ¹H NMR (400 MHz, DMSO-d₆) δ=11.00 (br s, 1H), 10.25 (s, 1H), 7.80 (s, 1H), 7.72-7.62 (m, 2H), 7.24 (t, J=73.2 Hz, 1H), 7.23-7.19 (m, 1H), 7.16 (s, 1H), 6.76 (td, J=2.2, 9.7 Hz, 1H), 5.30 (s, 2H), 5.13 (dd, J=5.1, 13.3 Hz, 1H), 4.51-4.45 (m, 1H), 4.38-4.32 (m, 1H), 2.95-2.86 (m, 1H), 2.64-2.60 (m, 1H), 2.46-2.36 (m, 1H), 2.05-1.98 (m, 1H). MS (ESI) m/z 478.1 [M+H]⁺

Step 1: To a solution of methanol (3.00 mL, 74.1 mmol, 3.93 eq) in N-methyl-pyrrolidone (10.0 mL) was added sodium hydride (60% dispersion in mineral oil) (830 mg, 20.7 mmol, 1.10 eq). The reaction was stirred at 0° C. for 1 h, then 1,3-difluoro-5-nitro-benzene (3.00 g, 18.9 mmol, 1.00 eq) was added. The reaction was stirred at 25° C. for another 11 h. The reaction was quenched with 1 M hydrochloric acid (40.0 mL) and extracted with ethyl acetate (3×30.0 mL). The combined organic layers were washed with brine (15.0 mL), dried over anhydrous sodium sulfate, filtered, and concentrated under reduced pressure give a residue. The residue was purified by silica gel column chromatography (petroleum ether/ethyl acetate=10/1 to 1/1) to afford 1-fluoro-3-methoxy-5-nitro-benzene.

Step 2: To a solution of 1-fluoro-3-methoxy-5-nitro-benzene (2.50 g, 14.6 mmol, 1.00 eq) in dichloromethane (15.0 mL) was added boron tribromide (11.0 g, 43.8 mmol, 4.22 mL, 3.00 eq) at −78° C. The reaction was stirred at −78° C. for 1 h, and then at 25° C. for 11 h. The reaction was quenched with methanol (30.0 mL) and concentrated under reduced pressure to give a residue. The residue was purified by silica gel column chromatography (petroleum ether/ethyl acetate=10/1 to 5/1) to afford 3-fluoro-5-nitro-phenol.

Step 3: A solution of 3-fluoro-5-nitro-phenol (0.500 g, 3.18 mmol, 1.00 eq), sodium 2-chloro-2,2-difluoro-acetate (1.46 g, 9.55 mmol, 3.00 eq) and potassium carbonate (879 mg, 6.37 mmol, 2.00 eq) in dimethylformamide (10.0 mL) and water (2.00 mL) was stirred at 100° C. for 12 h. The mixture was poured into water (20.0 mL). The aqueous phase was extracted with ethyl acetate (3×30 mL). The combined organic layers were washed with brine (30.0 mL), dried over anhydrous sodium sulfate, filtered, and concentrated under reduced pressure to give a residue. The residue was purified by silica gel column chromatography (petroleum ether/ethyl acetate=10/1 to 5/1) to afford 1-(difluoromethoxy)-3-fluoro-5-nitrobenzene.

Step 4: A mixture of 1-(difluoromethoxy)-3-fluoro-5-nitrobenzene (700 mg, 3.38 mmol, 1.00 eq), ferrous powder (566 mg, 10.1 mmol, 3.00 eq) and ammonium chloride (904 mg, 16.9 mmol, 5.00 eq) in methanol (6.00 mL) and water (3.00 mL) was stirred at 80° C. for 1 h. The mixture was filtered, and the filtrate was concentrated under reduced pressure to afford 3-(difluoromethoxy)-5-fluoroaniline.

Step 5: To a solution of 3-(difluoromethoxy)-5-fluoroaniline (300 mg, 1.69 mmol, 1.00 eq) in acetonitrile (20.0 mL) were added pyridine (0.68 mL, 8.42 mmol, 4.97 eq) and phenyl chloroformate (0.25 mL, 2.00 mmol, 1.18 eq) at 0° C. The reaction was stirred at 25° C. for 1 h. The mixture was concentrated and the obtained residue was purified by standard methods to afford phenyl (3-(difluoromethoxy)-5-fluorophenyl)carbamate.

Compound 232: General procedure A with variant i) was used for the preparation from compound VIII employing phenyl (3-(difluoromethoxy)-4-fluorophenyl) carbamate. 1H NMR (400 MHz, DMSO-d₆) δ=11.00 (s, 1H), 10.04 (s, 1H), 7.80 (s, 1H), 7.71-7.67 (m, 1H), 7.67-7.63 (m, 1H), 7.58 (br d, J=6.6 Hz, 1H), 7.37-7.27 (m, 2H), 7.21 (t, J=73.2 Hz, 1H), 5.29 (s, 2H), 5.13 (br d, J=8.2 Hz, 1H), 4.46 (s, 1H), 4.40-4.30 (m, 1H), 2.92 (br d, J=1.6 Hz, 1H), 2.64-2.59 (m, 1H), 2.46-2.37 (m, 1H), 2.06-1.98 (m, 1H). MS (ESI) m/z 478.1 [M+H]⁺

Step 1: To a solution of 2-fluoro-5-nitrophenol (500 mg, 3.18 mmol, 1.00 eq) and methyl 2-chloro-2,2-difluoroacetate (2.43 g, 15.9 mmol, 5.00 eq) in dimethylformamide (20.0 mL) and water (3.00 mL) was added potassium carbonate (880 mg, 6.37 mmol, 2.00 eq). The reaction was stirred at 100° C. for 12 h. Water (50.0 mL) was added, and the aqueous layer was extracted with ethyl acetate (3×20.0 mL). The organic layers were gathered, dried over anhydrous sodium sulfate, filtered, and concentrated under reduced pressure to give a residue. The residue was purified by silica gel column chromatography (petroleum ether/ethyl acetate=20/1 to 10/1) to afford 2-(difluoromethoxy)-1-fluoro-4-nitrobenzene.

Step 2: To a solution of 2-(difluoromethoxy)-1-fluoro-4-nitrobenzene (400 mg, 1.93 mmol, 1.00 eq) in methanol (9.00 mL) and water (3.00 mL) were added ammonium chloride (516 mg, 9.66 mmol, 5.00 eq) and ferrous powder (539 mg, 9.66 mmol, 5.00 eq). The reaction was stirred at 80° C. for 2 h. The mixture was filtered and concentrated under reduced pressure to afford 3-(difluoromethoxy)-4-fluoroaniline.

Step 3: To a solution of 3-(difluoromethoxy)-4-fluoroaniline (110 mg, 621 μmol, 1.00 eq) in acetonitrile (2.00 mL) were added pyridine (0.25 mL, 3.11 mmol, 5.00 eq) and phenyl chloroformate (0.08 mL, 0.68 mmol, 1.10 eq) at 0° C. The reaction was stirred at 0° C. for 0.5 h. The mixture was filtered, and the filtrate was concentrated and the obtained residue was purified by standard methods to afford phenyl (3-(difluoromethoxy)-4-fluorophenyl)carbamate.

Compound 233: General procedure A with variant i) was used for the preparation from compound VIII employing phenyl (3-ethoxy-4-methylphenyl) carbamate. ¹H NMR (400 MHz, DMSO-d₆) δ=10.99 (br s, 1H), 9.68 (br s, 1H), 7.79 (s, 1H), 7.69-7.65 (m, 1H), 7.65-7.61 (m, 1H), 7.14 (br s, 1H), 7.00 (d, J=8.2 Hz, 1H), 6.90 (br d, J=7.9 Hz, 1H), 5.25 (s, 2H), 5.12 (dd, J=5.1, 13.3 Hz, 1H), 4.50-4.43 (m, 1H), 4.38-4.29 (m, 1H), 3.95 (q, J=6.8 Hz, 2H), 2.96-2.86 (m, 1H), 2.63-2.56 (m, 1H), 2.40 (br dd, J=4.5, 13.1 Hz, 1H), 2.07-2.05 (m, 3H), 2.01 (dt, J=2.1, 6.2 Hz, 1H), 1.33 (t, J=6.9 Hz, 3H). MS (ESI) m/z 452.2 [M+H]⁺

Step 1: To a solution of 2-methyl-5-nitrophenol (1.00 g, 6.53 mmol, 1.00 eq) and iodoethane (0.57 mL, 7.18 mmol, 1.10 eq) in dimethylformamide (10.0 mL) was added potassium carbonate (2.71 g, 19.6 mmol, 3.00 eq) under nitrogen. The reaction was stirred at 40° C. for 2 h under nitrogen atmosphere. The mixture was poured into water (20.0 mL) and extracted with ethyl acetate (3×20.0 mL). The combined organic layers were gathered, washed with brine (10.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-ethoxy-1-methyl-4-nitrobenzene.

Step 2: To a solution of 2-ethoxy-1-methyl-4-nitrobenzene (0.900 g, 4.97 mmol, 1.00 eq) in methanol (10.0 mL) and water (10.0 mL) were added iron powder (1.39 g, 24.8 mmol, 5.00 eq) and ammonium chloride (1.33 g, 24.8 mmol, 5.00 eq). The reaction was stirred at 80° C. for 2 h. The mixture was concentrated under reduced pressure to give a slurry. The slurry was poured into saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-ethoxy-4-methylaniline.

Step 3: To a solution of 3-ethoxy-4-methylaniline (500 mg, 3.31 mmol, 1.00 eq) in acetonitrile (5.00 mL) were added pyridine (1.33 mL, 16.5 mmol, 5.00 eq) and phenyl chloroformate (0.50 mL, 3.97 mmol, 1.20 eq) at 0° C. The reaction was stirred at 25° C. for 1 h. The mixture was concentrated and the obtained residue was purified by standard methods to afford phenyl (3-ethoxy-4-methylphenyl)carbamate.

Compound 234: General procedure A with variant i) was used for the preparation from compound VIII employing phenyl (4-methyl-3-(morpholinomethyl)-5-(trifluoromethoxy)phenyl) carbamate. ¹H NMR (400 MHz, DMSO-d₆) 5=11.00 (s, 1H), 10.26 (br s, 1H), 10.20 (br s, 1H), 7.80 (s, 1H), 7.74-7.62 (m, 3H), 7.58 (br s, 1H), 5.31 (s, 2H), 5.12 (dd, J=5.1, 13.3 Hz, 1H), 4.52-4.30 (m, 4H), 3.94 (br d, J=12.1 Hz, 2H), 3.75 (brt, J=11.6 Hz, 2H), 3.30-3.15 (m, 3H), 2.99-2.84 (m, 1H), 2.61 (br d, J=17.0 Hz, 1H), 2.43 (br dd, J=8.7, 13.3 Hz, 2H), 2.35-2.30 (m, 3H), 2.07-1.98 (m, 1H). MS (ESI) m/z 591.1 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and filtered. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.10 eq) and quinolin-8-ol (563 μL, 3.26 mmol, 0.20 eq) in water (3.00 mL) and dimethylsulfoxide (3.00 mL) was added a solution of potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to give a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: To a solution of (3-hydroxy-2-methyl-5-nitro-phenyl)-morpholino-methanone (1.30 g, 4.88 mmol, 1.00 eq), silver trifluoromethanesulfonate (6.27 g, 24.4 mmol, 5.00 eq), 1-(chloromethyl)-4-fluoro-1,4-diazoniabicyclo[2.2.2]octane bis(tetrafluoroborate) (3.46 g, 9.77 mmol, 2.00 eq), N-fluorobenzenesulfonimide (3.08 g, 9.77 mmol, 2.00 eq) and caesium fluoride (4.45 g, 29.3 mmol, 1.08 mL, 6.00 eq) in toluene (130 mL) were added trimethyl(trifluoromethyl)silane (3.47 g, 24.4 mmol, 5.00 eq) and 2-fluoropyridine (2.10 mL, 24.4 mmol, 5.00 eq) under nitrogen. The reaction was stirred at 20° C. for 12 h under nitrogen. The mixture was filtered, and the filtrate was concentrated under reduced pressure to give a residue. The residue was diluted with water (100 mL) and extracted with ethyl acetate (100 mL). The organic layer was washed with water (20.0 mL), dried over anhydrous sodium sulfate, filtered, and the filtrate was concentrated under reduced pressure to give a residue. The residue was purified by silica gel column chromatography (petroleum ether/ethyl acetate=1/0 to 2/1) to afford (2-methyl-5-nitro-3-(trifluoromethoxy)phenyl)(morpholino)methanone.

Step 5: To a solution of (2-methyl-5-nitro-3-(trifluoromethoxy)phenyl)-morpholino-methanone (900 mg, 2.69 mmol, 1.00 eq) in tetrahydrofuran (15.0 mL) was added borane dimethyl sulfide complex (10.0 M, 539 μL, 2.00 eq) at 0° C. The reaction was stirred at 60° C. for 30 min. The mixture was quenched with methanol (2.00 mL) and concentrated under reduced pressure to give a residue. The residue was purified by reversed phase preparative HPLC to afford 4-(2-methyl-5-nitro-3-(trifluoromethoxy)benzyl)morpholine.

Step 6: To a solution of 4-(2-methyl-5-nitro-3-(trifluoromethoxy)benzyl)morpholine (400 mg, 1.25 mmol, 1.00 eq) in methanol (5.00 mL) and water (5.00 mL) was added iron powder (488 mg, 8.74 mmol, 7.00 eq) and ammonium chloride (468 mg, 8.74 mmol, 7.00 eq). The reaction was stirred at 80° C. for 2 h. The mixture was diluted with saturated sodium carbonate (1.00 mL) and extracted with ethyl acetate (2×10.0 mL). The combined organic layers were washed with water (5.00 mL), dried over anhydrous sodium sulfate, filtered, and the filtrate was concentrated under reduced pressure to give a residue. The residue was purified by silica gel column chromatography (petroleum ether/ethyl acetate=3/1 to 1/1) to afford 4-methyl-3-(morpholinomethyl)-5-(trifluoromethoxy)aniline.

Step 7: To a solution of 4-methyl-3-(morpholinomethyl)-5-(trifluoromethoxy)aniline (100 mg, 344 μmol, 1.00 eq) and potassium carbonate (57.1 mg, 413 μmol, 1.20 eq) in acetone (1.00 mL) was added phenyl chloroformate (47 μL, 379μ, 1.10 eq) at 25° C. The reaction was stirred at 25° C. for 1 h. The mixture was diluted with water (6.00 mL) and extracted with ethyl acetate (10.0 mL). The organic layer was dried over anhydrous sodium sulfate, filtered, and the filtrate was concentrated to afford phenyl (4-methyl-3-(morpholinomethyl)-5-(trifluoromethoxy)phenyl)carbamate.

Compound 235: General procedure A with variant ii) was used for the preparation from compound VIII employing 1-(pyridin-2-yl)piperidin-4-amine. 1H NMR (400 MHz, DMSO-d₆) δ=11.00 (s, 1H), 8.35-8.27 (m, 1H), 8.09 (dd, J=1.4, 4.9 Hz, 1H), 7.72 (s, 1H), 7.61 (s, 2H), 7.53-7.47 (m, 1H), 7.36 (br d, J=7.5 Hz, 1H), 6.83 (d, J=8.4 Hz, 1H), 6.59 (dd, J=5.0, 6.8 Hz, 1H), 5.17-5.08 (m, 3H), 4.50-4.43 (m, 1H), 4.37-4.30 (m, 1H), 4.20 (br d, J=13.1 Hz, 2H), 3.58 (br s, 1H), 2.92 (br s, 3H), 2.63-2.58 (m, 1H), 2.43-2.38 (m, 1H), 2.04-1.98 (m, 1H), 1.80 (br d, J=10.3 Hz, 2H), 1.41-1.30 (m, 2H). MS (ESI) m/z 478.2 [M+H]⁺

Step 1: A solution of 2-fluoropyridine (1.79 mL, 20.8 mmol, 1.00 eq), tert-butyl piperidin-4-ylcarbamate (5.00 g, 25.0 mmol, 1.20 eq) and potassium carbonate (5.75 g, 41.6 mmol, 2.00 eq) in dimethylacetamide (20.0 mL) was stirred at 120° C. for 12 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=5/1 to 3/1) to afford tert-butyl (1-(pyridin-2-yl)piperidin-4-yl)carbamate.

Step 2: A solution of tert-butyl (1-(pyridin-2-yl)piperidin-4-yl)carbamate (1.27 g, 4.58 mmol, 1.00 eq) and hydrochloric acid/ethyl acetate (8.00 mL) in ethyl acetate (24.0 mL) was stirred at 25° C. for 2 h. The mixture was concentrated and the obtained residue was purified by standard methods and filtered to afford 1-(pyridin-2-yl)piperidin-4-amine.

Compound 236: General procedure A with variant ii) was used for the preparation from compound VIII employing 1-phenylpiperazine. ¹H NMR (400 MHz, DMSO-d₆) δ=11.00 (s, 1H), 7.74 (s, 1H), 7.69-7.59 (m, 2H), 7.27-7.18 (m, 2H), 6.95 (d, J=7.9 Hz, 2H), 6.81 (t, J=7.3 Hz, 1H), 5.22 (s, 2H), 5.12 (dd, J=5.1, 13.3 Hz, 1H), 4.52-4.42 (m, 1H), 4.39-4.29 (m, 1H), 3.55 (br s, 4H), 3.17-3.09 (m, 4H), 2.92 (ddd, J=5.4, 13.7, 17.4 Hz, 1H), 2.63-2.58 (m, 1H), 2.44-2.35 (m, 1H), 2.06-1.98 (m, 1H). MS (ESI) m/z 463.2 [M+H]⁺

Compound 237: General procedure A with variant i) was used for the preparation from compound VIII employing phenyl (3-methoxy-4-methylphenyl)carbamate. ¹H NMR (400 MHz, DMSO-d₆) δ=11.00 (br s, 1H), 9.71 (br s, 1H), 7.80 (s, 1H), 7.71-7.67 (m, 1H), 7.66-7.61 (m, 1H), 7.17 (s, 1H), 7.01 (d, J=8.2 Hz, 1H), 6.93 (s, 1H), 5.27 (s, 2H), 5.16-5.10 (m, 1H), 4.46 (s, 1H), 4.37 (s, 1H), 3.73 (s, 3H), 2.97-2.86 (m, 1H), 2.58 (br s, 1H), 2.43-2.35 (m, 1H), 2.07 (s, 3H), 2.03 (br s, 1H). MS (ESI) m/z 438.1 [M+H]⁺

To a solution of 3-methoxy-4-methylaniline (500 mg, 3.64 mmol, 1.00 eq) in acetonitrile (5.00 mL) were added pyridine (1.47 mL, 18.2 mmol, 5.00 eq) and phenyl chloroformate (0.91 mL, 7.29 mmol, 2.00 eq) at 0° C. The reaction was stirred at 0° C. for 1 h. The mixture was concentrated under reduced pressure to give a residue. The residue was purified by reversed phase preparative HPLC. The desired fraction was collected and concentrated under reduced pressure to give concentrated solution. The solution was diluted with water/ethyl acetate (40.0 ml/80.0 ml). The organic layer was separated, dried over anhydrous sodium sulfate, filtered, and concentrated by standard methods to afford phenyl (3-methoxy-4-methylphenyl)carbamate.

Compound 238: General procedure A with variant i) was used for the preparation from compound VIII employing phenyl (4-chloro-2-methoxyphenyl)carbamate. ¹H NMR (400 MHz, DMSO-d₆) δ=11.08-10.95 (m, 1H), 8.86 (s, 1H), 7.81 (s, 1H), 7.71-7.61 (m, 3H), 7.10 (d, J=2.3 Hz, 1H), 6.98 (dd, J=2.3, 8.6 Hz, 1H), 5.26 (s, 2H), 5.18-5.10 (m, 1H), 4.51-4.44 (m, 1H), 4.39-4.30 (m, 1H), 3.83 (s, 3H), 2.97-2.88 (m, 1H), 2.62-2.59 (m, 1H), 2.41 (br dd, J=4.4, 12.9 Hz, 1H), 2.06-1.98 (m, 1H). MS (ESI) m/z 458.1 [M+H]⁺

To a solution of 4-chloro-2-methoxyaniline (1.00 g, 6.35 mmol, 1.00 eq) in acetonitrile (5.00 mL) were added pyridine (2.57 mL, 31.8 mmol, 5.01 eq) and phenyl chloroformate (0.88 mL 7.03 mmol, 1.11 eq) at 0° C. The reaction was stirred at 0° C. for 1 h. The mixture was concentrated and the obtained residue was purified by standard methods to afford phenyl (4-chloro-2-methoxyphenyl)carbamate.

Compound 239: General procedure A with variant i) was used for the preparation with from compound VIII employing phenyl (4-cyclopropylphenyl)carbamate. ¹H NMR (400 MHz, DMSO-d₆) δ=10.99 (s, 1H), 9.68 (br s, 1H), 7.78 (s, 1H), 7.71-7.59 (m, 2H), 7.34 (br d, J=8.4 Hz, 2H), 6.98 (d, J=8.6 Hz, 2H), 5.25 (s, 2H), 5.18-5.08 (m, 1H), 4.53-4.43 (m, 1H), 4.37-4.29 (m, 1H), 2.99-2.85 (m, 1H), 2.65-2.56 (m, 1H), 2.47-2.34 (m, 1H), 2.05-1.97 (m, 1H), 1.89-1.79 (m, 1H), 0.92-0.84 (m, 2H), 0.62-0.55 (m, 2H). MS (ESI) m/z 434.3 [M+H]⁺

To a solution of 4-cyclopropylaniline (500 mg, 3.75 mmol, 1.00 eq) in acetonitrile (5.00 mL) was added pyridine (0.91 mL, 11.3 mmol, 3.00 eq) and phenyl chloroformate (0.56 mL, 4.50 mmol, 1.20 eq). The reaction was stirred at 25° C. for 2 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (4-cyclopropylphenyl)carbamate.

Compound 240: General procedure A with variant i) was used for the preparation from compound VIII employing phenyl (3-chloro-5-((1-methylpyrrolidin-3-yl)methoxy)phenyl)carbamate. ¹H NMR (400 MHz, DMSO-d₆) δ=10.99 (s, 1H), 9.99 (s, 1H), 8.15 (s, 1H), 7.79 (s, 1H), 7.70-7.61 (m, 2H), 7.13 (s, 1H), 7.06 (s, 1H), 6.67 (t, J=1.9 Hz, 1H), 5.28 (s, 2H), 5.12 (dd, J=5.2, 13.1 Hz, 1H), 4.52-4.43 (m, 1H), 4.38-4.30 (m, 1H), 3.85 (br dd, J=4.7, 7.0 Hz, 2H), 2.96-2.88 (m, 1H), 2.74-2.68 (m, 1H), 2.62 (br s, 1H), 2.58 (br s, 2H), 2.55-2.52 (m, 2H), 2.43-2.38 (m, 1H), 2.35 (s, 3H), 2.04-1.91 (m, 2H), 1.53 (br dd, J=6.3, 12.5 Hz, 1H). MS (ESI) m/z 541.2 [M+H]⁺

Step 1: To a solution of tert-butyl 3-(hydroxymethyl)pyrrolidine-1-carboxylate (3.00 g, 14.9 mmol, 1.00 eq) and triethylamine (3.77 g, 37.3 mmol, 5.19 mL, 2.50 eq) in dichloromethane (30.0 mL) at 0° C. was added methylsulfamoyl chloride (1.50 mL, 19.4 mmol, 1.30 eq) dropwise under nitrogen atmosphere. The reaction was stirred at 25° C. for 2 h. The mixture was diluted with ethyl acetate (100 mL) and water (150 mL). The organic layer was separated, and the aqueous layer was extracted with ethyl acetate (3×100 mL). The organic layers were gathered, dried over sodium sulfate, filtered, and concentrated under reduced pressure to afford tert-butyl 3-(((methylsulfonyl)oxy)methyl)pyrrolidine-1-carboxylate.

Step 2: To a solution of tert-butyl 3-(((methylsulfonyl)oxy)methyl) pyrrolidine-1-carboxylate (3.00 g, 10.8 mmol, 1.00 eq) in dimethylformamide (30.0 mL) was added 3-chloro-5-nitrophenol (2.05 g, 11.8 mmol, 1.10 eq) and cesium carbonate (10.5 g, 32.2 mmol, 3.00 eq). The reaction was stirred at 80° C. for 12 h. The mixture was diluted with ethyl acetate (100 mL) and water (100 mL). The organic layer was separated, and the aqueous layer was extracted with ethyl acetate (3×80.0 mL). The organic layers were gathered, dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl) pyrrolidine-1-carboxylate.

Step 3: To a solution of tert-butyl 3-((3-chloro-5-nitrophenoxy)methyl)pyrrolidine-1-carboxylate (1.10 g, 3.08 mmol, 1.00 eq) in ethyl acetate (5.00 mL) was added hydrochloric acid in ethyl acetate (4 M, 10 mL). The reaction was stirred at 25° C. for 2 h. The mixture was concentrated under reduced pressure to afford 3-((3-chloro-5-nitrophenoxy) methyl)pyrrolidine.

Step 4: To a solution of 3-((3-chloro-5-nitrophenoxy)methyl)pyrrolidine (1.50 g, 5.84 mmol, 1.00 eq) in 2,2,2-trifluoroethanol (10.0 mL) was added paraformaldehyde (0.80 mL, 29.2 mmol, 5.00 eq). The reaction was stirred at 60° C. for 0.5 h. Sodium borohydride (442 mg, 11.7 mmol, 2.00 eq) was added in portions, and the reaction was stirred at 60° C. for 1 h. The reaction was quenched with saturated ammonium chloride solution (10.0 mL) and concentrated under reduced pressure to give a residue. The residue was purified by reversed phase preparative HPLC to afford 3-((3-chloro-5-nitrophenoxy)methyl)-1-methylpyrrolidine.

Step 5: To a solution of 3-((3-chloro-5-nitrophenoxy)methyl)-1-methylpyrrolidine (1.20 g, 4.43 mmol, 1.00 eq) in methanol (6.00 mL) and water (6.00 mL) was added ammonium chloride (1.66 g, 31.0 mmol, 7.00 eq) and iron powder (1.73 g, 31.0 mmol, 7.00 eq). The reaction was stirred at 80° C. for 2 h. The mixture was filtered over Celite, and the filtrate was concentrated under reduced pressure to afford 3-chloro-5-((1-methylpyrrolidin-3-yl)methoxy)aniline.

Step 6: To a solution of phenyl chloroformate (0.28 mL, 2.24 mmol, 1.20 eq) in acetonitrile (5.00 mL) was added pyridine (0.45 mL, 5.61 mmol, 3.00 eq) and 3-chloro-5-((1-methylpyrrolidin-3-yl) methoxy)aniline (450 mg, 1.87 mmol, 1.00 eq). The reaction was stirred at 25° C. for 2 h. The mixture was concentrated and the obtained residue was purified by standard methods to afford phenyl (3-chloro-5-((1-methylpyrrolidin-3-yl) methoxy)phenyl)carbamate.

Compound 241: General procedure A with variant i) was used for the preparation from compound VIII employing phenyl (3-fluoro-4-methyl-5-(morpholinomethyl)phenyl)carbamate. ¹H NMR (400 MHz, DMSO-d₆) δ=10.99 (s, 1H), 10.90 (br s, 1H), 10.12 (s, 1H), 7.79 (s, 1H), 7.71-7.61 (m, 2H), 7.50 (s, 1H), 7.36 (br d, J=11.7 Hz, 1H), 5.29 (s, 2H), 5.11 (dd, J=5.1, 13.3 Hz, 1H), 4.51-4.43 (m, 1H), 4.38-4.29 (m, 3H), 3.90-3.81 (m, 4H), 3.34-3.14 (m, 4H), 2.97-2.86 (m, 1H), 2.60 (br d, J=17.7 Hz, 1H), 2.46-2.36 (m, 1H), 2.29 (d, J=1.7 Hz, 3H), 2.09-1.94 (m, 1H). MS (ESI) m/z 525.3 [M+H]⁺

Step 1: To a solution of 3-fluoro-2-methylbenzoic acid (10.0 g, 64.9 mmol, 1.00 eq) in sulfuric acid (100 mL) was added potassium nitrate (7.22 g, 71.4 mmol, 1.10 eq) in portions at 0° C. The reaction was stirred at 0° C. for 1 h. The mixture was poured into water (100 mL) and the resulting precipitate was collected by filtration. The filter cake was dried under vacuum, then added to water (100 mL) and extracted with ethyl acetate (3×80.0 mL). The combined organic layers were washed with brine (60.0 mL), dried over sodium sulfate, filtered, and concentrated under reduced pressure to afford 3-fluoro-2-methyl-5-nitrobenzoic acid.

Step 2: To a solution of 3-fluoro-2-methyl-5-nitrobenzoic acid (11.0 g, 55.2 mmol, 1.00 eq) in tetrahydrofuran (100 mL) was added borane dimethyl sulfide complex (10 M, 11.0 mL, 2.00 eq) at 0° C. The reaction was stirred at 25° C. for 12 h. The mixture was poured into methanol (200 mL) and concentrated under reduced pressure to give a residue. Water (150 mL) was added, and the pH was adjusted to pH=10 by addition of 15% sodium hydroxide solution. The aqueous layer was extracted with ethyl acetate (3×80.0 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=4/1) to afford (3-fluoro-2-methyl-5-nitrophenyl)methanol.

Step 3: To a solution of (3-fluoro-2-methyl-5-nitrophenyl)methanol (870 mg, 4.70 mmol, 1.00 eq) in dichloromethane (10.0 mL) was added thionyl chloride (1.70 mL, 23.5 mmol, 5.00 eq) dropwise at 0° C. The reaction was stirred at 25° C. for 12 h. The mixture was concentrated under reduced pressure to afford 1-(chloromethyl)-3-fluoro-2-methyl-5-nitrobenzene.

Step 4: To a solution of 1-(chloromethyl)-3-fluoro-2-methyl-5-nitrobenzene (950 mg, 4.67 mmol, 1.00 eq) and triethylamine (1.62 mL, 11.7 mmol, 2.50 eq) in acetonitrile (10.0 mL) was added morpholine (0.51 mL, 5.83 mmol, 1.25 eq) dropwise. The reaction was stirred at 25° C. for 12 h. The mixture was concentrated under reduced pressure to give a residue. The residue was diluted with water (80.0 mL) and extracted with ethyl acetate (3×60.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=4/1) to afford 4-(3-fluoro-2-methyl-5-nitrobenzyl)morpholine.

Step 5: To a solution of 4-(3-fluoro-2-methyl-5-nitrobenzyl)morpholine (1.00 g, 3.93 mmol, 1.00 eq) and ammonium chloride (1.05 g, 19.7 mmol, 5.00 eq) in methanol (8.00 mL) and water (2.00 mL) was added iron powder (1.10 g, 19.7 mmol, 5.00 eq) in portions. The reaction was stirred at 80° C. for 2 h. The mixture was filtered, and the filtrate was diluted with water (80.0 mL) and extracted with ethyl acetate (3×60.0 mL). The combined organic layers were washed with brine (40.0 mL), dried over sodium sulfate, filtered, and concentrated under reduced pressure to afford 3-fluoro-4-methyl-5-(morpholinomethyl)aniline.

Step 6: To a solution of 3-fluoro-4-methyl-5-(morpholinomethyl)aniline (300 mg, 1.34 mmol, 1.00 eq) and pyridine (0.54 mL, 6.69 mmol, 5.00 eq) in acetonitrile (5.00 mL) was added phenyl chloroformate (0.20 mL, 1.61 mmol, 1.20 eq) dropwise at 0° C. The reaction was stirred at 25° C. for 12 h. The mixture was diluted with water (50.0 mL) and extracted with ethyl acetate (3×40.0 mL). The combined organic layers were washed with brine (30.0 mL), dried over sodium sulfate, filtered, and concentrated and the obtained residue was purified by standard methods to afford phenyl (3-fluoro-4-methyl-5-(morpholinomethyl)phenyl)carbamate.

Compound 242: General procedure A with variant i) was used for the preparation from compound VIII employing phenyl (6-phenylpyridin-3-yl)carbamate. ¹H NMR (400 MHz, DMSO-d₆) δ=10.99 (s, 1H), 10.23 (br s, 1H), 8.75 (s, 1H), 8.06-7.95 (m, 4H), 7.82 (s, 1H), 7.73-7.68 (m, 1H), 7.67-7.62 (m, 1H), 7.51-7.45 (m, 2H), 7.44-7.39 (m, 1H), 5.33 (s, 2H), 5.13 (dd, J=5.1, 13.2 Hz, 1H), 4.51-4.45 (m, 1H), 4.38-4.32 (m, 1H), 2.97-2.86 (m, 1H), 2.60 (br d, J=17.2 Hz, 1H), 2.41 (br dd, J=4.4, 12.9 Hz, 1H), 2.05-1.97 (m, 1H). MS (ESI) m/z 471.2 [M+H]⁺

To a solution of 6-phenylpyridin-3-amine (300 mg, 1.76 mmol, 1.00 eq) and pyridine (0.71 mL, 8.81 mmol, 5.00 eq) in acetonitrile (5.00 mL) was added phenyl chloroformate (0.26 mL, 2.12 mmol, 1.20 eq) in portions at 0° C. The reaction was stirred at 25° C. for 1 h. The mixture was concentrated and the obtained residue was purified by standard methods to afford phenyl (6-phenylpyridin-3-yl)carbamate.

Compound 243: General procedure A with variant i) was used for the preparation from compound VIII employing phenyl (6-(tert-butyl)pyridin-3-yl)carbamate. ¹H NMR (400 MHz, DMSO-d₆) δ=11.01 (s, 1H), 10.80-10.56 (m, 1H), 8.91-8.80 (m, 1H), 8.29 (br d, J=8.5 Hz, 1H), 7.87 (br d, J=8.6 Hz, 1H), 7.82 (s, 1H), 7.73-7.69 (m, 1H), 7.68-7.63 (m, 1H), 5.35 (s, 2H), 5.13 (dd, J=5.1, 13.3 Hz, 1H), 4.53-4.43 (m, 1H), 4.38-4.32 (m, 1H), 2.97-2.87 (m, 1H), 2.66-2.57 (m, 1H), 2.47-2.35 (m, 1H), 2.06-1.97 (m, 1H), 1.45-1.37 (m, 9H). MS (ESI) m/z 451.2 [M+H]⁺

To a solution of 6-(tert-butyl)pyridin-3-amine (150 mg, 1.00 mmol, 1.00 eq) and pyridine (0.40 mL, 5.00 mmol, 5.00 eq) in acetonitrile (5.00 mL) was added phenyl chloroformate (0.15 mL, 1.20 mmol, 1.20 eq). The reaction was stirred at 25° C. for 1 h. The mixture was concentrated and the obtained residue was purified by standard methods to afford phenyl (6-(tert-butyl)pyridin-3-yl)carbamate.

### Compound 244:

Step 1: A solution of 3-chloro-2-methyl-5-nitrophenol (260 mg, 1.39 mmol, 1.00 eq), tert-butyl 3-((methylsulfonyl)oxy)pyrrolidine-1-carboxylate (441 mg, 1.66 mmol, 1.20 eq) and potassium carbonate (575 mg, 4.16 mmol, 3.00 eq) in dimethylformamide (10.0 mL) was stirred at 80° C. for 4 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=1/0 to 5/1) to afford tert-butyl 3-(3-chloro-2-methyl-5-nitrophenoxy)pyrrolidine-1-carboxylate.

Step 2: To a mixture of tert-butyl 3-(3-chloro-2-methyl-5-nitrophenoxy)pyrrolidine-1-carboxylate (430 mg, 1.21 mmol, 1.00 eq), iron powder (337 mg, 6.03 mmol, 5.00 eq) and ammonium chloride (322 g, 6.03 mmol, 5.00 eq) in methanol (10.0 mL) was added water (10.0 mL) at 25° C. The reaction was stirred at 80° C. for 2 h. The mixture was concentrated under reduced pressure to give a residue. The residue was added to saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford tert-butyl 3-(5-amino-3-chloro-2-methylphenoxy)pyrrolidine-1-carboxylate.

Step 3: To a solution of tert-butyl 3-(5-amino-3-chloro-2-methylphenoxy)pyrrolidine-1-carboxylate (170 mg, 520 μmol, 1.00 eq) and pyridine (0.21 mL, 2.60 mmol, 5.00 eq) in acetonitrile (5.00 mL) was added phenyl chloroformate (78.0 μL, 623 μmol, 1.20 eq) in portions at 0° C. The reaction was stirred at 25° C. for 1 h. The mixture was concentrated under reduced pressure to give a residue, which was purified by reversed phase preparative HPLC to afford tert-butyl 3-(3-chloro-2-methyl-5-((phenoxycarbonyl)amino)phenoxy)pyrrolidine-1-carboxylate.

Step 4: To a solution of 3-(6-(hydroxymethyl)-1-oxoisoindolin-2-yl)piperidine-2,6-dione VIII (100 mg, 365 μmol, 1.00 eq) and tert-butyl 3-(3-chloro-2-methyl-5-((phenoxycarbonyl)amino)phenoxy)pyrrolidine-1-carboxylate (179 mg, 401 μmol, 1.10 eq) in dimethylformamide (3.00 mL) was added sodium hydride (60% dispersion in mineral oil) (30.0 mg, 750 μmol, 2.06 eq) in portions at 0° C. The reaction was stirred at 25° C. for 1 h. The reaction was quenched with hydrochloric acid (1 M, 0.50 mL), and the solvents were removed under reduced pressure to give a residue. The residue was purified by reversed phase preparative HPLC to afford tert-butyl 3-(3-chloro-5-((((2-(2,6-dioxopiperidin-3-yl)-3-oxoisoindolin-5-yl)methoxy)carbonyl)amino)-2-methylphenoxy)pyrrolidine-1-carboxylate.

Step 5: To a solution of tert-butyl 3-(3-chloro-5-((((2-(2,6-dioxopiperidin-3-yl)-3-oxoisoindolin-5-yl)methoxy)carbonyl)amino)-2-methylphenoxy)pyrrolidine-1-carboxylate (120 mg, 191 μmol, 1.00 eq) in ethyl acetate (10.0 mL) was added hydrochloric acid/ethyl acetate (4 M, 4.62 mL, 96.5 eq). The reaction was stirred at 25° C. for 1 h. The mixture was concentrated to give a residue, and purified by a standard method to afford Compound 244 (hydrochloride). ¹H NMR (400 MHz, DMSO-d₆) δ=10.99 (s, 1H), 9.95 (br s, 1H), 9.54 (br s, 1H), 9.49-9.38 (m, 1H), 7.79 (s, 1H), 7.70-7.62 (m, 2H), 7.25-7.11 (m, 2H), 5.28 (s, 2H), 5.12 (dd, J=5.1, 13.2 Hz, 1H), 5.02 (br s, 1H), 4.52-4.44 (m, 1H), 4.38-4.31 (m, 1H), 3.51-3.43 (m, 2H), 3.37-3.31 (m, 2H), 3.27 (br dd, J=6.9, 9.9 Hz, 1H), 2.97-2.85 (m, 1H), 2.60 (br d, J=16.9 Hz, 1H), 2.40 (br dd, J=4.5, 13.1 Hz, 1H), 2.23-2.16 (m, 1H), 2.14 (s, 3H), 2.06-1.95 (m, 1H). MS (ESI) m/z 527.2 [M+H]⁺

Compound 245: General procedure A with variant i) was used for the preparation with from compound VIII employing phenyl (3-chloro-5-ethyl-4-methylphenyl)carbamate. ¹H NMR (400 MHz, DMSO-d₆) δ=10.99 (s, 1H), 9.85 (s, 1H), 7.79 (s, 1H), 7.70-7.61 (m, 2H), 7.46 (s, 1H), 7.22 (d, J=1.9 Hz, 1H), 5.27 (s, 2H), 5.12 (dd, J=5.1, 13.3 Hz, 1H), 4.50-4.44 (m, 1H), 4.37-4.31 (m, 1H), 2.99-2.86 (m, 1H), 2.65-2.61 (m, 1H), 2.60-2.56 (m, 2H), 2.46-2.36 (m, 1H), 2.22 (s, 3H), 2.06-1.98 (m, 1H), 1.11 (t, J=7.5 Hz, 3H). MS (ESI) m/z 470.2 [M+H]⁺

Step 1: To a solution of 2-chloro-1-methyl-4-nitrobenzene (0.60 mL, 2.91 mmol, 1.00 eq) in sulfuric acid (5.00 mL, 98% purity) was added N-iodosuccinimide (787 mg, 3.50 mmol, 1.20 eq) in portions. The reaction was stirred at 60° C. for 1 h. The mixture was quenched with sodium carbonate (10%, 100 mL) and extracted with ethyl acetate (3×30.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 1-chloro-3-iodo-2-methyl-5-nitrobenzene.

Step 2: To a solution of 1-chloro-3-iodo-2-methyl-5-nitrobenzene (760 mg, 2.55 mmol, 1.00 eq) in toluene (15.0 mL) were added diisopropylethylamine (1.34 mL, 7.66 mmol, 3.00 eq), [1,1′-bis(diphenylphosphino)ferrocene]dichloropalladium(II) (187 mg, 256 μmol, 0.10 eq) and potassium vinyltrifluoroborate (685 mg, 5.11 mmol, 2.00 eq) in portions under nitrogen. The reaction was stirred at 110° C. for 12 h. The mixture was concentrated under reduced pressure to give a residue, which was purified by silica gel column chromatography (petroleum ether/ethyl acetate=1/0 to 10/1) to afford 1-chloro-2-methyl-5-nitro-3-vinylbenzene.

Step 3: To a solution of 1-chloro-2-methyl-5-nitro-3-vinylbenzene (370 mg, 1.87 mmol, 1.00 eq) in tetrahydrofuran (6.00 mL) and ethyl acetate (6.00 mL) were added zinc chloride (12.7 μL, 271 μmol, 0.140 eq) and palladium on activated carbon (10%) (wetted with ca. 55% water) (50.0 mg) in portions under H₂ (15 Psi). The reaction was stirred at 25° C. for 1 h. The mixture was filtered, and the filtrate was concentrated under reduced pressure to give a residue. The residue was purified by silica gel column chromatography (petroleum ether/ethyl acetate=20/1 to 5/1) to afford 3-chloro-5-ethyl-4-methylaniline.

Step 4: To a solution of 3-chloro-5-ethyl-4-methylaniline (125 mg, 737 μmol, 1.00 eq) in acetonitrile (8.00 mL) were added pyridine (0.30 mL, 3.72 mmol, 5.04 eq) and phenyl chloroformate (0.11 mL, 886 μmol, 1.20 eq) in portions at 0° C. The reaction was stirred at 25° C. for 1 h. The mixture was concentrated and the obtained residue was purified by standard methods to afford phenyl (3-chloro-5-ethyl-4-methylphenyl)carbamate.

Compound 246: General procedure A with variant i) was used for the preparation from compound VIII employing phenyl (3-chloro-5-((1-methylpyrrolidin-3-yl)oxy)phenyl)carbamate. ¹H NMR (400 MHz, DMSO-d₆) δ=10.99 (s, 1H), 9.99 (s, 1H), 8.15 (s, 1H), 7.79 (s, 1H), 7.70-7.62 (m, 2H), 7.15 (s, 1H), 6.99 (s, 1H), 6.60 (s, 1H), 5.28 (s, 2H), 5.12 (dd, J=5.0, 13.2 Hz, 1H), 4.82 (br d, J=5.4 Hz, 1H), 4.50-4.44 (m, 1H), 4.37-4.31 (m, 1H), 2.97-2.88 (m, 2H), 2.77 (br d, J=5.5 Hz, 1H), 2.72 (br d, J=6.1 Hz, 2H), 2.62 (brd, J=1.5 Hz, 1H), 2.58 (br s, 2H), 2.29 (s, 3H), 2.04-1.98 (m, 1H), 1.80-1.71 (m, 1H). MS (ESI) m/z 527.2 [M+H]⁺

Step 1: To a solution of tert-butyl 3-(3-chloro-5-nitro-phenoxy)pyrrolidine-1-carboxylate (2.00 g, 5.83 mmol, 1.00 eq) in ethyl acetate (10.0 mL) was added hydrochloric acid in ethyl acetate (4 M, 20.0 mL, 13.7 eq). The reaction was stirred at 25° C. for 1 h. The mixture was concentrated under reduced pressure to afford 3-(3-chloro-5-nitrophenoxy)pyrrolidine.

Step 2: To a solution of 3-(3-chloro-5-nitrophenoxy)pyrrolidine (1.00 g, 4.12 mmol, 1.00 eq) in methanol (6.00 mL) was added paraformaldehyde 37% purity (6.00 mL, 80.6 mmol, 19.60 eq), acetic acid (0.23 mL, 4.11 mmol, 1.00 eq) and sodium cyanoborohydride (1.29 g, 20.6 mmol, 5.00 eq) in portions. The reaction was stirred at 25° C. for 1 h. The mixture was concentrated under reduced pressure to give a residue. The residue was purified first by silica gel column chromatography (petroleum ether/ethyl acetate to ethyl acetate/methanol=3/1 to 0/1), then by reversed phase preparative HPLC, to afford 3-(3-chloro-5-nitrophenoxy)-1-methylpyrrolidine.

Step 3: To a solution of 3-(3-chloro-5-nitrophenoxy)-1-methylpyrrolidine (460 mg, 1.79 mmol, 1.00 eq) in methanol (15.0 mL) and water (8.00 mL) was added iron powder (300 mg, 5.37 mmol, 3.00 eq) and ammonium chloride (479 mg, 8.95 mmol, 5.00 eq) in portions. The reaction was stirred at 80° C. for 1 h. The mixture was concentrated under reduced pressure to give a residue, which was diluted with water (50.0 mL) and extracted with ethyl acetate (3×20.0 mL). The combined organic layers were washed with water, dried over anhydrous sodium sulfate, filtered, and concentrated under reduced pressure to afford 3-chloro-5-((1-methylpyrrolidin-3-yl)oxy)aniline.

Step 4: To a solution of 3-chloro-5-((1-methylpyrrolidin-3-yl)oxy)aniline (290 mg, 1.28 mmol, 1.00 eq) in acetonitrile (5.00 mL) was added pyridine (0.52 mL, 6.39 mmol, 5.00 eq) and phenyl chloroformate (0.19 mL, 1.54 mmol, 1.20 eq) in portions. The reaction was stirred at 25° C. for 1 h. The mixture was concentrated and the obtained residue was purified by standard methods to afford phenyl (3-chloro-5-((1-methylpyrrolidin-3-yl)oxy) phenyl) carbamate.

Compound 247: General procedure A with variant i) was used for the preparation from compound VIII employing (4-(tert-butyl)phenyl)carbamate. ¹H NMR (400 MHz, DMSO-d₆) δ=10.99 (s, 1H), 9.70 (br s, 1H), 7.79 (s, 1H), 7.72-7.60 (m, 2H), 7.38 (br d, J=8.7 Hz, 2H), 7.32-7.25 (m, 2H), 5.26 (s, 2H), 5.16-5.08 (m, 1H), 4.52-4.43 (m, 1H), 4.39-4.28 (m, 1H), 2.98-2.85 (m, 1H), 2.64-2.63 (m, 1H), 2.65-2.56 (m, 1H), 2.47-2.33 (m, 1H), 2.07-1.97 (m, 1H), 1.24 (s, 9H). MS (ESI) m/z 450.2 [M+H]⁺

To a solution of phenyl carbamate (0.63 mL, 5.03 mmol, 1.50 eq) and pyridine (0.81 mL, 10.1 mmol, 3.00 eq) in acetonitrile (5.00 mL) was added 4-(tert-butyl)aniline (0.53 mL, 3.35 mmol, 1.00 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×50.0 mL). The combined organic layers were dried over sodium sulfate, filtered, and concentrated and the obtained residue was purified by standard methods to afford phenyl (4-(tert-butyl)phenyl)carbamate.

Compound 248: General procedure A with variant i) was used for the preparation from compound VIII employing phenyl (3-chloro-4-methyl-5-(2-morpholinoethoxy)phenyl) carbamate. ¹H NMR (400 MHz, DMSO-d₆) δ=11.00 (s, 1H), 9.89 (s, 1H), 8.20 (s, 1H), 7.79 (s, 1H), 7.71-7.60 (m, 2H), 7.14 (br d, J=19.5 Hz, 2H), 5.27 (s, 2H), 5.13 (dd, J=5.1, 13.3 Hz, 1H), 4.51-4.43 (m, 1H), 4.38-4.30 (m, 1H), 4.03 (t, J=5.7 Hz, 2H), 3.58-3.55 (m, 4H), 2.96-2.87 (m, 1H), 2.72 (t, J=5.6 Hz, 2H), 2.60 (br d, J=17.5 Hz, 1H), 2.47 (br d, J=4.6 Hz, 4H), 2.35 (br d, J=4.5 Hz, 1H), 2.12 (s, 3H), 2.05-1.97 (m, 1H). MS (ESI) m/z 571.2 [M+H]⁺

Step 1: A solution of 2-chloro-1-methyl-4-nitrobenzene (12.1 mL, 58.3 mmol, 1.00 eq) and N-iodosuccinimide (14.4 g, 64.1 mmol, 1.10 eq) in sulfuric acid (100 mL) was stirred at 60° C. for 2 h. The reaction was quenched by addition of ice water (200 mL) at 0° C. The aqueous layer was extracted with ethyl acetate (3×50.0 mL). The combined organic layers were washed with brine (100 mL), dried over sodium sulfate, filtered, and concentrated under reduced pressure to give a residue. The residue was purified by silica gel column chromatography (petroleum ether/ethyl acetate=1/0) to afford 1-chloro-3-iodo-2-methyl-5-nitrobenzene.

Step 2: A solution of 1-chloro-3-iodo-2-methyl-5-nitrobenzene (7.80 g, 26.2 mmol, 1.00 eq), potassium hydroxide (4.41 g, 78.7 mmol, 3.00 eq), tris(dibenzylidenethyl acetatecetone)dipalladium(0) (1.20 g, 1.31 mmol, 0.05 eq) and 2-di-tert-butylphosphino-2′,4′,6′-triisopropylbiphenyl (557 mg, 1.31 mmol, 0.050 eq) in dioxane (80.0 mL) and water (16.0 mL) was stirred at 80° C. for 12 h under nitrogen atmosphere. The mixture was acidified to pH˜4 and concentrated under reduced pressure to give a residue. Brine (200 mL) was added, and the aqueous layer was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=1/0 to 17/3) to afford 3-chloro-2-methyl-5-nitrophenol.

Step 3: A suspension of 3-chloro-2-methyl-5-nitrophenol (500 mg, 2.67 mmol, 1.00 eq), 4-(2-chloroethyl) morpholine (479 mg, 3.20 mmol, 1.20 eq), potassium carbonate (553 mg, 4.00 mmol, 1.50 eq) and potassium iodide (133 mg, 0.80 mmol, 0.30 eq) in dimethylformamide (5.00 mL) was stirred at 80° C. for 1 h. The mixture was poured into brine (50.0 mL) and extracted with ethyl acetate (3×15.0 mL). The combined organic layers were dried over sodium sulfate, filtered, and concentrated under reduced pressure to afford 4-(2-(3-chloro-2-methyl-5-nitrophenoxy)ethyl)morpholine.

Step 4: To a solution of 4-(2-(3-chloro-2-methyl-5-nitrophenoxy)ethyl)morpholine (570 mg, 1.90 mmol, 1.00 eq) in methanol (5.00 mL) and water (1.00 mL) were added iron powder (529 mg, 9.48 mmol, 5.00 eq) and ammonium chloride (507 mg, 9.48 mmol, 5.00 eq). The reaction was stirred at 80° C. for 12 h under nitrogen. The mixture was filtered, and the filtrate was concentrated under reduced pressure to give a residue. The residue was treated with brine (5.00 mL) and saturated aqueous sodium bicarbonate (5.00 mL). The aqueous layer was extracted with ethyl acetate (3×10.0 mL), dried over sodium sulfate, filtered, and concentrated under reduced pressure to afford 3-chloro-4-methyl-5-(2-morpholinoethoxy)aniline.

Step 5: To a solution of 3-chloro-4-methyl-5-(2-morpholinoethoxy)aniline (300 mg, 1.11 mmol, 1.00 eq) in acetonitrile (10.0 mL) were added pyridine (0.45 mL, 5.54 mmol, 5.00 eq) and phenyl chloroformate (0.17 mL, 1.33 mmol, 1.20 eq) at 0° C. The reaction was stirred at 25° C. for 1 h. The mixture was concentrated and the obtained residue was purified by standard methods to afford phenyl (3-chloro-4-methyl-5-(2-morpholinoethoxy)phenyl)carbamate.

Compound 249: General procedure A with variant i) was used for the preparation from compound VIII employing phenyl (6-ethyl-5-methylpyridin-3-yl)carbamate. ¹H NMR (400 MHz, DMSO-d₆) δ=11.11-10.94 (m, 1H), 10.81-10.66 (m, 1H), 8.67 (d, J=1.6 Hz, 1H), 8.20 (br s, 1H), 7.82 (s, 1H), 7.74-7.59 (m, 2H), 5.35 (s, 2H), 5.19-5.02 (m, 1H), 4.53-4.44 (m, 1H), 4.40-4.27 (m, 1H), 3.04-2.90 (m, 3H), 2.61 (br d, J=16.5 Hz, 1H), 2.43 (s, 3H), 2.42-2.35 (m, 1H), 2.07-1.97 (m, 1H), 1.24 (t, J=7.6 Hz, 3H). MS (ESI) m/z 437.1 [M+H]⁺

Step 1: To a solution of 2-chloro-3-methyl-5-nitropyridine (2.00 g, 11.6 mmol, 1.00 eq), ethylboronic acid (2.14 g, 29.0 mmol, 2.50 eq) and potassium carbonate (4.81 g, 34.8 mmol, 3.00 eq) in dioxane (20.0 mL) was added tetrakis[triphenylphosphine]palladium(0) (1.34 g, 1.16 mmol, 0.100 eq) at 20° C. The reaction was stirred at 110° C. under nitrogen for 12 h. The mixture was concentrated under reduced pressure to give a residue. The residue was purified by silica gel column chromatography (petroleum ether/ethyl acetate=1/0 to 20/1) to afford 2-ethyl-3-methyl-5-nitropyridine.

Step 2: A mixture of 2-ethyl-3-methyl-5-nitropyridine (800 mg, 4.81 mmol, 1.00 eq), iron powder (806 mg, 14.4 mmol, 3.00 eq) and ammonium chloride (1.29 g, 24.0 mmol, 5.00 eq) in methanol (8.00 mL) and water (4.00 mL) was stirred at 80° C. for 1 h. The mixture was filtered, and the filtrate was concentrated under reduced pressure to give a residue. The residue was added to water (100 mL) and the solution was stirred for 10 min, then extracted with ethyl acetate (3×50.0 mL). The combined organic layers were washed with brine (100 mL), dried over anhydrous sodium sulfate, filtered, and concentrated under reduced pressure to afford 6-ethyl-5-methylpyridin-3-amine.

Step 3: To a solution of 6-ethyl-5-methyl-pyridin-3-amine (300 mg, 2.20 mmol, 1 eq) and pyridine (0.89 mL, 11.0 mmol, 5.00 eq) in acetonitrile (5.00 mL) was added phenyl chloroformate (0.41 mL, 3.30 mmol, 1.50 eq). The reaction was stirred at 25° C. for 1 h. The mixture was concentrated under reduced pressure to give a residue. The residue was added to water (80.0 mL) and stirred for 10 min, then extracted with ethyl acetate (3×40.0 mL). The combined organic layers were washed with brine (80.0 mL), dried over anhydrous sodium sulfate, filtered, and concentrated and the obtained residue was purified by standard methods to afford phenyl (6-ethyl-5-methylpyridin-3-yl)carbamate.

Compound 250: General procedure A with variant i) was used for the preparation from compound VIII employing phenyl (4-(pyridin-2-yl)phenyl)carbamate. ¹H NMR (400 MHz, DMSO-d₆) δ=11.00 (s, 1H), 10.01 (s, 1H), 8.62 (d, J=4.1 Hz, 1H), 8.29 (s, 1H), 8.03 (d, J=8.8 Hz, 2H), 7.93-7.88 (m, 1H), 7.87-7.79 (m, 2H), 7.73-7.69 (m, 1H), 7.67-7.63 (m, 1H), 7.60 (d, J=8.6 Hz, 2H), 7.29 (dd, J=5.3, 6.7 Hz, 1H), 5.30 (s, 2H), 5.13 (dd, J=5.0, 13.3 Hz, 1H), 4.53-4.44 (m, 1H), 4.41-4.30 (m, 1H), 2.96-2.86 (m, 1H), 2.65-2.58 (m, 1H), 2.41 (dq, J=4.4, 13.2 Hz, 1H), 2.06-1.99 (m, 1H). MS (ESI) m/z 471.2 [M+H]⁺

Step 1: To a solution of (4-nitrophenyl)boronic acid (1.41 g, 8.44 mmol, 1.00 eq) and 2-bromopyridine (1.20 mL, 12.7 mmol, 1.50 eq) in ethanol (35.0 mL) were added potassium carbonate (2.33 g, 16.9 mmol, 2.00 eq) and tetrakis[triphenylphosphine]palladium(0) (1.95 g, 1.69 mmol, 0.20 eq) in one portion. The reaction was stirred at 90° C. under nitrogen for 12 h. The mixture was diluted with water (30.0 mL) and exacted with ethyl acetate (3×30 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 2-(4-nitrophenyl)pyridine.

Step 2: To a solution of 2-(4-nitrophenyl)pyridine (600 mg, 3.00 mmol, 1.00 eq) in methanol (6.00 mL) and water (3.00 mL) were added ferrous powder (502 mg, 8.99 mmol, 3.00 eq) and ammonium chloride (802 mg, 15.0 mmol, 5.00 eq). The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 4-(2-pyridyl)aniline.

Step 3: To a solution of 4-(pyridin-2-yl)aniline (200 mg, 1.18 mmol, 1.00 eq) and pyridine (0.28 mL, 3.53 mmol, 3.00 eq) in acetonitrile (3.00 mL) was added phenyl chloroformate (0.22 mL, 1.76 mmol, 1.50 eq) dropwise at 0° C. The reaction was stirred at 25° C. for 12 h. The mixture was concentrated and the obtained residue was purified by standard methods to afford phenyl (4-(pyridin-2-yl)phenyl)carbamate.

Compound 251: General procedure A with variant i) was used for the preparation from compound VIII employing phenyl(5-methoxy-6-methylpyridin-3-yl)carbamate. ¹H NMR (400 MHz, DMSO-d₆) δ=10.99 (s, 1H), 10.70 (br s, 1H), 8.34 (s, 1H), 7.98 (s, 1H), 7.81 (s, 1H), 7.73-7.62 (m, 2H), 5.35 (s, 2H), 5.12 (dd, J=5.1, 13.3 Hz, 1H), 4.51-4.45 (m, 1H), 4.37-4.30 (m, 1H), 3.93 (s, 3H), 2.97-2.86 (m, 1H), 2.60 (br d, J=17.6 Hz, 1H), 2.50-2.49 (m, 3H), 2.40 (dq, J=4.4, 13.2 Hz, 1H), 2.07-1.95 (m, 1H). MS (ESI) m/z 439.1 [M+H]⁺

Step 1: To a solution of 2-chloro-3-methoxy-5-nitro-pyridine (3.00 g, 15.9 mmol, 1.00 eq), methylboronic acid (1.90 g, 31.8 mmol, 2.00 eq) and potassium carbonate (6.60 g, 47.7 mmol, 3.00 eq) in dioxane (30.0 mL) was added tetrakis[triphenylphosphine]palladium(0) (1.84 g, 1.59 mmol, 0.10 eq) at 25° C. The reaction was stirred at 110° C. for 12 h under nitrogen. The mixture was concentrated under reduced pressure to give a residue. The residue was purified by silica gel column chromatography (petroleum ether/ethyl acetate=1/0 to 30/1) to afford 3-methoxy-2-methyl-5-nitropyridine.

Step 2: A mixture of 3-methoxy-2-methyl-5-nitropyridine (500 mg, 2.97 mmol, 1.00 eq), iron powder (498 mg, 8.92 mmol, 3.00 eq), and ammonium chloride (795 mg, 14.9 mmol, 5.00 eq) in methanol (4.00 mL) and water (2.00 mL) was stirred at 80° C. for 1 h. The mixture was filtered, and the filtrate was concentrated under reduced pressure to give a residue. The residue was added to water (80.0 mL) and stirred for 10 min. The solution was extracted with ethyl acetate (3×50.0 mL). The combined organic layers were washed with brine (80.0 mL), dried over anhydrous sodium sulfate, filtered, and concentrated under reduced pressure to afford 5-methoxy-6-methylpyridin-3-amine.

Step 3: To a solution of 5-methoxy-6-methylpyridin-3-amine (200 mg, 1.45 mmol, 1.00 eq), and pyridine (0.58 mL, 7.24 mmol, 5.00 eq) in acetonitrile (4.00 mL) was added phenyl chloroformate (0.22 mL, 1.74 mmol, 1.20 eq) dropwise at 0° C. The reaction was stirred at 25° C. for 12 h. The mixture was concentrated under reduced pressure to give a residue. The residue was diluted with water (50.0 mL) and extracted with ethyl acetate (3×40.0 mL). The combined organic layers were washed with brine (20.0 mL), dried over sodium sulfate, filtered, and concentrated and the obtained residue was purified by standard methods to afford phenyl (5-methoxy-6-methylpyridin-3-yl)carbamate.

### Compound 252:

Step 1: To a solution of 2-chloro-1-methyl-4-nitrobenzene (10.0 g, 58.3 mmol, 12.1 mL, 1.00 eq) in sulfuric acid (100 mL) was added N-iodosuccinimide (14.4 g, 64.1 mmol, 1.10 eq) in portions. The reaction was stirred at 60° C. for 2 h. The mixture was poured into water (300 mL) and extracted with ethyl acetate (3×80.0 mL). The combined organic layers were washed with brine (50 mL) and dried over anhydrous sodium sulfate, filtered, and concentrated under reduced pressure to afford 1-chloro-3-iodo-2-methyl-5-nitrobenzene.

Step 2: To a solution of 1-chloro-3-iodo-2-methyl-5-nitrobenzene (7.00 g, 23.5 mmol, 1.00 eq) and potassium hydroxide (3.96 g, 70.6 mmol, 3.00 eq) in dioxane (70.0 mL) and water (10.0 mL) were added tris(dibenzylideneacetone)dipalladium(0) (2.15 g, 2.35 mmol, 0.10 eq) and 2-di-tert-butylphosphino-2,4,6-triisopropylbiphenyl (999 mg, 2.35 mmol, 0.10 eq) under nitrogen. The reaction was stirred at 80° C. for 12 h. The mixture was diluted with water (300 mL) and extracted with ethyl acetate (3×80.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 3-chloro-2-methyl-5-nitrophenol.

Step 3: To a solution of 3-chloro-2-methyl-5-nitrophenol (1.00 g, 5.33 mmol, 1.00 eq) and tert-butyl 3-(((methylsulfonyl)oxy)methyl)pyrrolidine-1-carboxylate (1.64 g, 5.86 mmol, 1.10 eq) in dimethylformamide (10.0 mL) was added potassium carbonate (2.21 g, 15.9 mmol, 3.00 eq) in portions. The reaction was stirred at 80° C. for 12 h. The mixture was filtered, and the filtrate was concentrated under reduced pressure to give a residue. The residue was purified by reversed phase preparative HPLC to afford tert-butyl 3-((3-chloro-2-methyl-5-nitrophenoxy)methyl) pyrrolidine-1-carboxylate.

Step 4: To a solution of tert-butyl 3-((3-chloro-2-methyl-5-nitrophenoxy)methyl)pyrrolidine-1-carboxylate (400 mg, 1.08 mmol, 1.00 eq) and ammonium chloride (289 mg, 5.39 mmol, 5.00 eq) in methanol (4.00 mL) and water (4.00 mL) was added iron powder (181 mg, 3.24 mmol, 3.00 eq) in portions. The reaction was stirred at 80° C. for 2 h. The mixture was filtered, and the filtrate was concentrated under reduced pressure to give a residue. The residue was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford tert-butyl 3-((5-amino-3-chloro-2-methylphenoxy)methyl)pyrrolidine-1-carboxylate.

Step 5: To a solution of tert-butyl 3-((5-amino-3-chloro-2-methylphenoxy)methyl)pyrrolidine-1-carboxylate (200 mg, 587 μmol, 1.00 eq) and pyridine (0.14 mL, 1.76 mmol, 3.00 eq) in acetonitrile (5.00 mL) was added phenyl chloroformate (0.11 mL, 880 μmol, 1.50 eq) dropwise at 0° C. The reaction was stirred at 25° C. for 12 h. The mixture was concentrated under reduced pressure to give a residue. The residue was purified by reversed phase preparative HPLC to afford tert-butyl 3-((3-chloro-2-methyl-5-((phenoxycarbonyl)amino)phenoxy)methyl) pyrrolidine-1-carboxylate.

Step 6: To a solution of 3-(6-(hydroxymethyl)-1-oxoisoindolin-2-yl)piperidine-2,6-dione VIII (100 mg, 365 μmol, 1.00 eq) and tert-butyl 3-((3-chloro-2-methyl-5-((phenoxycarbonyl)amino)phenoxy)methyl)pyrrolidine-1-carboxylate (168 mg, 365 μmol, 1.00 eq) in dimethylformamide (1.00 mL) was added sodium hydride (60% dispersion in mineral oil) (29.2 mg, 729 μmol, 2.00 eq) in portions at 0° C. The reaction was stirred at 25° C. for 1 h. The mixture was quenched slowly with 1 M hydrochloric acid (2.00 mL) and concentrated under reduced pressure to afford tert-butyl 3-((3-chloro-5-((((2-(2,6-dioxopiperidin-3-yl)-3-oxoisoindolin-5-yl)methoxy)carbonyl)amino)-2-methylphenoxy)methyl)pyrrolidine-1-carboxylate.

Step 7: A solution of tert-butyl 3-((3-chloro-5-((((2-(2,6-dioxopiperidin-3-yl)-3-oxoisoindolin-5-yl)methoxy)carbonyl)amino)-2-methylphenoxy)methyl)pyrrolidine-1-carboxylate (200 mg, 312 μmol, 1.00 eq) in hydrochloric acid/ethyl acetate (3 M, 5.00 mL) was stirred at 25° C. for 1 h. The mixture was concentrated to give a residue. The residue was purified by a standard method to afford Compound 252. ¹H NMR (400 MHz, DMSO-d₆) δ=11.00 (s, 1H), 9.95 (s, 1H), 9.24 (br d, J=3.5 Hz, 2H), 7.79 (s, 1H), 7.71-7.62 (m, 2H), 7.17 (br d, J=13.4 Hz, 2H), 5.28 (s, 2H), 5.13 (dd, J=5.1, 13.3 Hz, 1H), 4.54-4.30 (m, 2H), 4.03-3.90 (m, 2H), 3.48-3.40 (m, 1H), 3.28-3.15 (m, 2H), 3.08-2.99 (m, 1H), 2.97-2.86 (m, 1H), 2.75 (td, J=7.2, 14.4 Hz, 1H), 2.61 (br d, J=17.6 Hz, 1H), 2.41 (dq, J=4.5, 13.2 Hz, 1H), 2.14 (s, 3H), 2.13-2.06 (m, 1H), 2.06-1.97 (m, 1H), 1.85-1.73 (m, 1H). MS (ESI) m/z 541.2 [M+H]⁺

Compound 253: General procedure A with variant i) was used for the preparation from compound VIII employing phenyl N-(6,7-dihydro-5H-cyclopenta[b]pyridin-3-yl)carbamate. ¹H NMR (400 MHz, DMSO-d₆) δ=11.01 (s, 1H), 10.59-10.34 (m, 1H), 8.67-8.54 (m, 1H), 8.18-8.01 (m, 1H), 7.86-7.77 (m, 1H), 7.73-7.69 (m, 1H), 7.67-7.63 (m, 1H), 5.33 (s, 2H), 5.13 (dd, J=5.2, 13.4 Hz, 1H), 4.53-4.44 (m, 1H), 4.39-4.32 (m, 1H), 3.02 (br dd, J=7.5, 18.5 Hz, 4H), 2.96-2.89 (m, 1H), 2.66-2.57 (m, 1H), 2.41 (dd, J=4.4, 13.2 Hz, 1H), 2.23-2.11 (m, 2H), 2.06-1.98 (m, 1H). MS (ESI) m/z 435.1 [M+H]⁺

Step 1: To a solution of 3-nitro-6,7-dihydro-5H-cyclopenta[b]pyridine (600 mg, 3.65 mmol, 1.00 eq) in methanol (6.00 mL) and water (3.00 mL) were added ferrous powder (612 mg, 11.0 mmol, 3.00 eq) and ammonium chloride (977 mg, 18.3 mmol, 5.00 eq). The reaction was stirred at 80° C. for 2 h. The mixture was filtered, and the filtrate was concentrated under reduced pressure to give a residue. Water (30.0 mL) was added to the residue, and the mixture was exacted with ethyl acetate (3×30.0 mL). The combined organic phases were washed with brine (2×10.0 mL), dried over sodium sulfate, filtered, and concentrated under reduced pressure to afford 6,7-dihydro-5H-cyclopenta[b]pyridin-3-amine.

Step 2: To a solution of 6,7-dihydro-5H-cyclopenta[b]pyridin-3-amine (350 mg, 2.61 mmol, 1.00 eq) and phenyl chloroformate (0.49 mL, 3.91 mmol, 1.50 eq) in methanol (3.00 mL) was added pyridine (0.63 mL, 7.83 mmol, 3.00 eq) in one portion. The reaction was stirred at 25° C. for 2 h. The mixture was diluted with water (30.0 mL) and exacted with ethyl acetate (3×30.0 mL). The combined organic phases were washed with brine (2×10.0 mL), dried over sodium sulfate, filtered, and concentrated and the obtained residue was purified by standard methods to afford phenyl (6,7-dihydro-5H-cyclopenta[b]pyridin-3-yl)carbamate. Compound 254: General procedure A with variant i) was used for the preparation from compound VIII employing phenyl (7,8-dihydro-5H-pyrano[4,3-b]pyridin-3-yl)carbamate. ¹H NMR (400 MHz, DMSO-d₆) δ=10.99 (s, 1H), 10.72 (s, 1H), 8.69 (s, 1H), 8.11 (s, 1H), 7.81 (s, 1H), 7.74-7.63 (m, 2H), 5.34 (s, 2H), 5.12 (dd, J=5.2, 13.2 Hz, 1H), 4.81 (s, 2H), 4.51-4.44 (m, 1H), 4.37-4.31 (m, 1H), 3.98 (t, J=5.6 Hz, 2H), 3.05 (s, 2H), 2.97-2.85 (m, 1H), 2.60 (d, J=17.6 Hz, 1H), 2.40 (dq, J=4.4, 13.2 Hz, 1H), 2.07-1.97 (m, 1H). MS (ESI) m/z 451.1 [M+H]⁺

To a solution of 7,8-dihydro-5H-pyrano[4,3-b]pyridin-3-amine (140 mg, 0.93 mmol, 1.00 eq) in acetonitrile (3.00 mL) were added phenyl chloroformate (0.13 mL, 1.03 mmol, 1.10 eq) and pyridine (0.23 mL, 2.80 mmol, 3.00 eq). The reaction was stirred at 25° C. for 2 h. The mixture was diluted with water 10.0 mL and ethyl acetate (10.0 mL). The aqueous layer was extracted with ethyl acetate (3×10.0 mL). The combined organic layers were dried over anhydrous sodium sulfate, filtered, and concentrated and the obtained residue was purified by standard methods to afford phenyl (7,8-dihydro-5H-pyrano[4,3-b] pyridin-3-yl)carbamate.

Compound 255: General procedure A with variant i) was used for the preparation from compound VIII employing phenyl (5-ethyl-6-methylpyridin-3-yl)carbamate. ¹H NMR (400 MHz, DMSO-d₆) δ=11.00 (s, 1H), 9.85 (br s, 1H), 8.34 (s, 1H), 8.15 (s, 1H), 7.80 (s, 1H), 7.74-7.61 (m, 3H), 5.28 (s, 2H), 5.13 (dd, J=5.2, 13.2 Hz, 1H), 4.53-4.43 (m, 1H), 4.40-4.30 (m, 1H), 2.96-2.88 (m, 1H), 2.63-2.59 (m, 1H), 2.57 (d, J=7.8 Hz, 2H), 2.46-2.41 (m, 1H), 2.38 (s, 3H), 2.06-1.98 (m, 1H), 1.14 (t, J=7.4 Hz, 3H). MS (ESI) m/z 437.3 [M+H]⁺

Step 1: To a solution of 3-bromo-2-methyl-5-nitropyridine (900 mg, 4.15 mmol, 1.00 eq), ethylboronic acid (919 mg, 12.4 mmol, 3.00 eq) and potassium carbonate (1.72 g, 12.4 mmol, 3.00 eq) in dioxane (10.0 mL) was added tetrakis[triphenylphosphine]palladium(0) (479 mg, 415 μmol, 0.100 eq) under nitrogen. The reaction was stirred at 110° C. for 12 h under nitrogen atmosphere. The mixture was concentrated under reduced pressure to give a residue. The residue was purified by silica gel column chromatography (petroleum ether/ethyl acetate=20/1 to 5/1) to afford 3-ethyl-2-methyl-5-nitropyridine.

Step 2: A mixture of 3-ethyl-2-methyl-5-nitropyridine (600 mg, 3.61 mmol, 1.00 eq), iron powder (605 mg, 10.8 mmol, 3.00 eq) and ammonium chloride (579 mg, 10.8 mmol, 3.00 eq) in methanol (10.0 mL) and water (10.0 mL) was stirred at 80° C. for 2 h. The mixture was concentrated under reduced pressure to give a residue. The residue was added to a saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 5-ethyl-6-methylpyridin-3-amine.

Step 3: To a solution of 5-ethyl-6-methylpyridin-3-amine (350 mg, 2.57 mmol, 1.00 eq) and pyridine (0.62 mL, 7.71 mmol, 3.00 eq) in acetonitrile (5.00 mL) was added phenyl chloroformate (0.48 mL, 3.85 mmol, 1.50 eq) at 25° C. The reaction was stirred at 25° C. for 2 h. The mixture was concentrated and the obtained residue was purified by standard methods to afford phenyl (5-ethyl-6-methylpyridin-3-yl)carbamate.

Compound 256: General procedure A with variant i) was used for the preparation from compound VIII employing phenyl (2,3-dihydro-1H-inden-5-yl)carbamate. ¹H NMR (400 MHz, DMSO-d₆) δ=10.99 (s, 1H), 9.66 (br s, 1H), 7.79 (s, 1H), 7.73-7.61 (m, 2H), 7.47-7.30 (m, 1H), 7.20 (br d, J=8.0 Hz, 1H), 7.14-7.06 (m, 1H), 5.26 (s, 2H), 5.13 (dd, J=5.1, 13.3 Hz, 1H), 4.55-4.43 (m, 1H), 4.39-4.31 (m, 1H), 2.92 (ddd, J=5.4, 13.6, 17.5 Hz, 1H), 2.80 (td, J=7.2, 12.7 Hz, 4H), 2.61 (br d, J=17.6 Hz, 1H), 2.41 (dd, J=4.3, 13.1 Hz, 1H), 2.06-1.89 (m, 3H). MS (ESI) m/z 434.1 [M+H]⁺

To a solution of 2,3-dihydro-1H-inden-5-amine (300 mg, 2.25 mmol, 1.00 eq) and pyridine (0.91 mL, 11.3 mmol, 5.00 eq) in acetonitrile (6.00 mL) was added phenyl chloroformate (0.42 mL, 3.38 mmol, 1.50 eq). The reaction was stirred at 25° C. for 1 h. The mixture concentrated under reduced pressure to give a residue. The residue was added to water (100 mL) and stirred for 10 min. The aqueous solution was extracted with ethyl acetate (3×40.0 mL). The combined organic layers were washed with brine (100 mL), dried over anhydrous sodium sulfate, filtered, and concentrated and the obtained residue was purified by standard methods to afford phenyl (2,3-dihydro-1H-inden-5-yl)carbamate.

Compound 257: General procedure A with variant i) was used for the preparation from compound VIII employing phenyl (3-fluoro-5-(trifluoromethoxy)phenyl)carbamate. ¹H NMR (400 MHz, DMSO-d₆) δ=10.99 (br s, 1H), 10.35 (s, 1H), 7.80 (s, 1H), 7.71-7.67 (m, 1H), 7.67-7.61 (m, 1H), 7.39-7.31 (m, 2H), 6.99 (br d, J=8.8 Hz, 1H), 5.30 (s, 2H), 5.13 (dd, J=5.1, 13.3 Hz, 1H), 4.52-4.42 (m, 1H), 4.38-4.30 (m, 1H), 2.98-2.85 (m, 1H), 2.63-2.57 (m, 1H), 2.43-2.35 (m, 1H), 2.06-1.97 (m, 1H). MS (ESI) m/z 496.3 [M+H]⁺

To a solution of 3-fluoro-5-(trifluoromethoxy)aniline (200 mg, 1.03 mmol, 1.00 eq) and pyridine (0.25 mL, 3.10 mmol, 3.02 eq) in acetonitrile (10.0 mL) was added phenyl chloroformate (0.15 mL, 1.23 mmol, 1.20 eq) in portions at 0° C. The reaction was stirred at 25° C. for 1 h. The mixture was concentrated and the obtained residue was purified by standard methods to afford phenyl (3-fluoro-5-(trifluoromethoxy)phenyl)carbamate.

Compound 258: General procedure A with variant i) was used for the preparation from compound VIII employing phenyl (4-(1-methylcyclopropyl)phenyl)carbamate. ¹H NMR (400 MHz, DMSO-d₆) δ=11.00 (s, 1H), 9.71 (br s, 1H), 7.79 (s, 1H), 7.70-7.66 (m, 1H), 7.66-7.60 (m, 1H), 7.37 (br d, J=8.4 Hz, 2H), 7.17-7.09 (m, 2H), 5.26 (s, 2H), 5.13 (dd, J=5.1, 13.3 Hz, 1H), 4.52-4.43 (m, 1H), 4.39-4.29 (m, 1H), 2.96-2.87 (m, 1H), 2.62 (br d, J=2.0 Hz, 1H), 2.43-2.35 (m, 1H), 2.05-1.98 (m, 1H), 1.34 (s, 3H), 0.80-0.73 (m, 2H), 0.73-0.66 (m, 2H). MS (ESI) m/z 448.2 [M+H]⁺

Step 1: To freshly distilled dichloromethane (50.0 mL) was added diethylzinc (1 M in toluene, 40.6 mL, 4.00 eq). The solution was cooled to −40° C., and diiodomethane (40.6 mL, 4.00 eq) in dichloromethane (10.0 mL) was added slowly. The mixture was stirred at −40° C. for 30 min, then trifluoroacetic acid (0.15 mL, 2.03 mmol, 0.20 eq) and N,N-dimethylacetamide (1.05 mL, 10.1 mmol, 1.00 eq) in dichloromethane (10.0 mL) were added. The reaction was stirred at −15° C. for 0.5 h, then 1-bromo-4-(prop-1-en-2-yl)benzene (2.00 g, 10.1 mmol, 1.00 eq) in dichloromethane (10.0 mL) was added at 0° C. The mixture was stirred at 25° C. for 12 h. The reaction was quenched with ice-water (50.0 mL) at 0° C. The organic layer was separated and concentrated under reduced pressure to give a residue. The residue was purified by silica gel column chromatography (petroleum ether 100%) to afford 1-bromo-4-(1-methylcyclopropyl)benzene.

Step 2: To a solution of 1-bromo-4-(1-methylcyclopropyl)benzene (2.00 g, 9.47 mmol, 1.00 eq) (crude) in tert-amyl alcohol (100 mL) were added tert-butyl carbamate (2.00 g, 17.1 mmol, 1.80 eq), methanesulfonato(2-di-tbutylphosphino-2,4,6-tri-ipropyl-1,1-biphenyl)(2-amino-1,1-biphenyl-2-yl)palladium(II) (600 mg, 755 μmol, 0.0800 eq) and sodium tert-butoxide (2 M in tetrahydrofuran, 14.0 mL, 2.96 eq). The reaction was stirred at 90° C. for 3 h under nitrogen. The mixture was diluted with ethyl acetate (200 mL) and water (200 mL). The organic layer was separated and concentrated under reduced pressure to give a residue. The residue was purified by reversed phase preparative HPLC to afford tert-butyl (4-(1-methylcyclopropyl)phenyl) carbamate.

Step 3: To a solution of tert-butyl (4-(1-methylcyclopropyl)phenyl) carbamate (520 mg, 2.10 mmol, 1.00 eq) in ethyl acetate (10.0 mL) was added hydrogen chloride/ethyl acetate (4 M, 10 mL, 19.0 eq). The reaction was stirred at 25° C. for 1 h. The mixture was concentrated under reduced pressure to afford 4-(1-methylcyclopropyl)aniline.

Step 4: To a solution of 4-(1-methylcyclopropyl)aniline (200 mg, 1.09 mmol, 1.00 eq, hydrochloric acid) in acetonitrile (50.0 mL) were added pyridine (0.50 mL, 6.19 mmol, 5.69 eq) and phenyl chloroformate (187 mg, 1.20 mmol, 1.10 eq) at 0° C. The reaction was stirred at 25° C. for 1 h. The mixture was concentrated and the obtained residue was purified by standard methods to afford phenyl (4-(1-methylcyclopropyl)phenyl)carbamate.

Compound 259: General procedure A with variant i) was used for the preparation from compound VIII employing phenyl (4-(trifluoromethoxy)pyridin-2-yl)carbamate. ¹H NMR (400 MHz, DMSO-d₆) δ=10.99 (br s, 1H), 10.80 (s, 1H), 8.38 (d, J=5.6 Hz, 1H), 7.82 (s, 2H), 7.74-7.59 (m, 2H), 7.08 (br d, J=5.5 Hz, 1H), 5.32 (s, 2H), 5.13 (br dd, J=5.0, 13.4 Hz, 1H), 4.50-4.43 (m, 1H), 4.38-4.31 (m, 1H), 2.95-2.88 (m, 1H), 2.62 (br d, J=2.1 Hz, 1H), 2.44-2.35 (m, 1H), 2.06-1.97 (m, 1H). MS (ESI) m/z 479.1 [M+H]⁺

To a solution of 4-(trifluoromethoxy)pyridin-2-amine (300 mg, 1.68 mmol, 1.00 eq) and pyridine (0.68 mL, 8.42 mmol, 5.00 eq) in acetonitrile (3.00 mL) was added phenyl chloroformate (0.25 mL, 2.02 mmol, 1.20 eq) dropwise at 0° C. The reaction was stirred at 25° C. for 3 h. The mixture was concentrated and the obtained residue was purified by standard methods to afford phenyl (4-(trifluoromethoxy)pyridin-2-yl)carbamate.

Compound 260: General procedure A with variant i) was used for the preparation from compound VIII employing phenyl (6-(pyrrolidin-1-yl)pyridin-3-yl)carbamate. ¹H NMR (400 MHz, DMSO-d₆) δ=11.00 (s, 1H), 9.43 (br s, 1H), 8.16 (s, 1H), 8.09 (br s, 1H), 7.77 (s, 1H), 7.64 (q, J=7.7 Hz, 2H), 7.57 (br d, J=9.4 Hz, 1H), 6.40 (d, J=8.9 Hz, 1H), 5.23 (s, 2H), 5.12 (dd, J=5.1, 13.3 Hz, 1H), 4.50-4.42 (m, 1H), 4.37-4.30 (m, 1H), 3.33 (br d, J=6.7 Hz, 4H), 2.93-2.86 (m, 1H), 2.63-2.58 (m, 1H), 2.43-2.36 (m, 1H), 2.04-1.98 (m, 1H), 1.94-1.89 (m, 4H). MS (ESI) m/z 464.2 [M+H]⁺

Step 1: To a solution of 2-chloro-5-nitropyridine (2.00 g, 12.6 mmol, 1.00 eq) in dimethylformamide (10.0 mL) were added pyrrolidine (1.58 mL, 18.9 mmol, 1.50 eq) and potassium carbonate (5.23 g, 37.8 mmol, 3.00 eq). The reaction was stirred at 60° C. for 1 h. After cooling to room temperature, the mixture was poured into ice-water (200 mL). The resulting yellow precipitate was collected by filtration and dried under reduced pressure to afford 5-nitro-2-(pyrrolidin-1-yl)pyridine.

Step 2: To a solution of 5-nitro-2-(pyrrolidin-1-yl)pyridine (2.40 g, 12.4 mmol, 1.00 eq) in methanol (240 mL) was added palladium on carbon (100 mg, 10% weight on C). The reaction was stirred at 25° C. for 1 h under hydrogen (15 Psi). The mixture was filtered, and the filtrate was concentrated under reduced pressure to afford 6-(pyrrolidin-1-yl)pyridin-3-amine.

Step 3: To a solution of 6-(pyrrolidin-1-yl)pyridin-3-amine (1.50 g, 9.19 mmol, 1.00 eq) in acetonitrile (15.0 mL) were added pyridine (3.71 mL, 45.9 mmol, 5.00 eq) and phenyl chloroformate (1.50 mL, 11.9 mmol, 1.30 eq). The reaction was stirred at 25° C. for 1 h. The mixture was concentrated and the obtained residue was purified by standard methods to afford phenyl (6-(pyrrolidin-1-yl)pyridin-3-yl)carbamate.

Compound 261: General procedure A with variant i) was used for the preparation from compound VIII employing phenyl (4-fluoro-3-(trifluoromethoxy)phenyl)carbamate. ¹H NMR (400 MHz, DMSO-d₆) δ=11.00 (br s, 1H), 10.16 (br s, 1H), 7.84-7.62 (m, 4H), 7.50-7.38 (m, 2H), 5.30 (br s, 2H), 5.14 (br dd, J=4.6, 13.2 Hz, 1H), 4.54-4.30 (m, 2H), 3.06-2.84 (m, 1H), 2.612 (br d, J=16.8 Hz, 1H), 2.46-2.38 (m, 1H), 2.08-1.96 (m, 1H). MS (ESI) m/z 496.1 [M+H]⁺

To a solution of 4-fluoro-3-(trifluoromethoxy)aniline (400 mg, 2.05 mmol, 1.00 eq) and phenyl chloroformate (0.28 mL, 2.26 mmol, 1.10 eq) in acetonitrile (3.00 mL) was added pyridine (0.50 mL, 6.15 mmol, 3.00 eq) in one portion. The reaction was stirred at 25° C. for 2 h. The mixture was diluted with water (30.0 mL) and exacted with ethyl acetate (3×30.0 mL). The combined organic phases were washed with brine (2×10.0 mL), dried over sodium sulfate, filtered, and concentrated and the obtained residue was purified by standard methods to afford phenyl (4-fluoro-3-(trifluoromethoxy)phenyl)carbamate.

Compound 262: General procedure A with variant i) was used for the preparation from compound VIII employing phenyl (4-(3-methyloxetan-3-yl)phenyl)carbamate. ¹H NMR (400 MHz, DMSO-d₆) δ=11.03 (s, 1H), 9.82 (br s, 1H), 7.83 (s, 1H), 7.74-7.65 (m, 2H), 7.48 (br d, J=8.5 Hz, 2H), 7.21 (d, J=8.6 Hz, 2H), 5.30 (s, 2H), 5.16 (dd, J=5.2, 13.3 Hz, 1H), 4.80 (d, J=5.5 Hz, 2H), 4.55-4.48 (m, 3H), 4.40 (s, 1H), 2.95 (s, 1H), 2.66 (br s, 1H), 2.44 (br dd, J=4.5, 12.9 Hz, 1H), 2.08-2.01 (m, 1H), 1.63 (s, 3H). MS (ESI) m/z 464.2 [M+H]⁺

Step 1: To a solution of diethyl 2-methylmalonate (13.6 g, 78.0 mmol, 13.3 mL, 1.10 eq) in dimethylformamide (80.0 mL) was added sodium hydride (60% dispersion in mineral oil) (3.40 g, 85.0 mmol, 1.20 eq) slowly at 0° C. The reaction was stirred at 0° C. for 0.5 h, then 1-fluoro-4-nitrobenzene (10.0 g, 70.8 mmol, 7.52 mL, 1.00 eq) was added. The mixture was stirred at 25° C. for 3 h. Water (200 mL) was added, and the mixture was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=10/1 to 8/1) to afford diethyl 2-methyl-2-(4-nitrophenyl)malonate.

Step 2: To a solution of diethyl 2-methyl-2-(4-nitrophenyl)malonate (7.00 g, 23.7 mmol, 1.00 eq) in tetrahydrofuran (15.0 mL) was added lithium aluminium hydride (953 mg, 25.1 mmol, 1.06 eq) slowly at 0° C. under nitrogen. The reaction was stirred at 0° C. for 3 h. The mixture was then partitioned between dichloromethane and 1 M hydrochloric acid. The organic layer was separated, and the aqueous layer was extracted with dichloromethane (3×50.0 mL). The combined organic layers were concentrated under reduced pressure to give a residue. The residue was purified by silica gel column chromatography (petroleum ether/ethyl acetate=10/1 to 0/1) to afford 2-methyl-2-(4-nitrophenyl)propane-1,3-diol.

Step 3: To a solution of 2-methyl-2-(4-nitrophenyl)propane-1,3-diol (200 mg, 947 μmol, 1.00 eq) in tetrahydrofuran (10.0 mL) was added n-butyllithium (2.5 M in hexane, 0.46 mL, 1.21 eq) and tosyl chloride (271 mg, 1.42 mmol, 1.50 eq) at 0° C. The reaction was stirred at 25° C. for 1 h. After cooling to 0° C., n-butyllithium (2.5 M in hexane, 0.46 mL, 1.21 eq) was added. The reaction was stirred at 65° C. for another 2 h. The reaction was quenched with saturated ammonium chloride (10 mL) and extracted with ethyl acetate (50.0 mL). The organic layer was separated and concentrated under reduced pressure to give a residue. The residue was purified by silica gel column chromatography (petroleum ether/ethyl acetate=3/1) to afford 3-methyl-3-(4-nitrophenyl)oxetane.

Step 4: To a solution of 3-methyl-3-(4-nitrophenyl)oxetane (56.0 mg, 290 μmol, 1.00 eq) in ethyl acetate (5.00 mL) was added wet palladium on carbon (10% weight on C) (20.0 mg). The reaction was stirred at 25° C. for 2 h under hydrogen (15.0 Psi). The mixture was filtered, and the filtrate was concentrated under reduced pressure to afford 4-(3-methyloxetan-3-yl)aniline.

Step 5: To a solution of 4-(3-methyloxetan-3-yl)aniline (60.0 mg, 368 μmol, 1.00 eq) in acetonitrile (3.00 mL) was added pyridine (0.15 mL, 1.84 mmol, 5.00 eq) and phenyl chloroformate (0.07 mL, 551 μmol, 1.50 eq) at 0° C. The reaction was stirred at 0° C. for 0.5 h. The mixture was filtered, and the filtrate was concentrated and the obtained residue was purified by standard methods to afford phenyl (4-(3-methyloxetan-3-yl)phenyl)carbamate.

Compound 263: General procedure A with variant i) was used for the preparation from compound VIII employing phenyl (2-ethylpyridin-4-yl)carbamate. ¹H NMR (400 MHz, DMSO-d₆) δ=11.37 (br d, J=10.6 Hz, 1H), 11.00 (s, 1H), 8.54 (d, J=6.7 Hz, 1H), 7.85-7.80 (m, 2H), 7.77-7.70 (m, 2H), 7.69-7.64 (m, 1H), 5.40 (s, 2H), 5.13 (dd, J=5.1, 13.3 Hz, 1H), 4.54-4.45 (m, 1H), 4.40-4.32 (m, 1H), 2.97-2.87 (m, 3H), 2.61 (br d, J=17.1 Hz, 1H), 2.41 (br dd, J=4.5, 13.0 Hz, 1H), 2.06-1.98 (m, 1H), 1.27 (t, J=7.5 Hz, 3H). MS (ESI) m/z 423.2 [M+H]⁺

To a solution of 2-ethylpyridin-4-amine (0.500 g, 4.09 mmol, 1.00 eq) and pyridine (1.65 mL, 20.4 mmol, 5.00 eq) in dimethylformamide (50.0 mL) was added phenyl chloroformate (0.77 mL, 6.19 mmol, 1.51 eq) at 0° C. The reaction was stirred at 0° C. for 12 h. The mixture was filtered and concentrated and the obtained residue was purified by standard methods to afford phenyl (2-ethylpyridin-4-yl)carbamate.

Compound 264: General procedure A with variant i) was used for the preparation from compound VIII employing phenyl (2-(piperidin-1-yl)pyrimidin-5-yl)carbamate. ¹H NMR (400 MHz, DMSO-d₆) δ=11.00 (s, 1H), 9.55 (br s, 1H), 8.39 (br s, 2H), 7.78 (s, 1H), 7.70-7.61 (m, 2H), 5.26 (s, 2H), 5.17-5.07 (m, 1H), 4.53-4.28 (m, 2H), 3.71-3.64 (m, 4H), 3.00-2.85 (m, 1H), 2.66-2.57 (m, 1H), 2.44-2.34 (m, 1H), 2.08-1.97 (m, 1H), 1.67-1.57 (m, 2H), 1.55-1.42 (m, 4H). MS (ESI) m/z 479.1 [M+H]⁺

Step 1: A solution of 2-chloro-5-nitro-pyrimidine (1.00 g, 6.27 mmol, 1.00 eq), piperidine (1.24 mL, 12.5 mmol, 2.00 eq) and potassium carbonate (2.60 g, 18.8 mmol, 3.00 eq) in dimethylformamide (5.00 mL) was stirred at 25° C. for 12 h. The mixture was diluted with ethyl acetate (100 mL) and water (100 mL). The organic layer was separated, and the aqueous layer was extracted with ethyl acetate (3×200 mL). The combined organic layers were dried over sodium sulfate, filtered, and concentrated under reduced pressure to afford 5-nitro-2-(piperidin-1-yl)pyrimidine.

Step 2: A mixture of 5-nitro-2-(1-piperidyl) pyrimidine (1.31 g, 6.27 mmol, 1.00 eq) and palladium on carbon (10% weight on C) (2.00 g, 6.27 mmol, 1.00 eq) in methanol (10.0 mL) was stirred at 25° C. for 12 h under hydrogen (15 Psi). The mixture was filtered over Celite, and the filtrate was concentrated under reduced pressure to afford 2-(piperidin-1-yl)pyrimidin-5-amine.

Step 3: To a solution of 2-(piperidin-1-yl)pyrimidin-5-amine (300 mg, 1.68 mmol, 1.00 eq), and pyridine (0.41 mL, 5.05 mmol, 3.00 eq) in acetonitrile (10.0 mL) was added phenyl chloroformate (0.32 mL, 2.52 mmol, 1.50 eq). The reaction was stirred at 25° C. for 12 h. The mixture was diluted with ethyl acetate (50.0 mL) and water (50.0 mL). The organic layer was separated, and the aqueous layer was extracted with ethyl acetate (3×100 mL). The combined organic layers were dried over sodium sulfate, filtered, and concentrated and the obtained residue was purified by standard methods to afford phenyl (2-(piperidin-1-yl)pyrimidin-5-yl)carbamate.

Compound 265: General procedure A with variant i) was used for the preparation from compound VIII employing phenyl (6-(8-oxa-3-azabicyclo[3.2.1]octan-3-yl)pyridin-3-yl)carbamate. ¹H NMR (400 MHz, DMSO-d₆) δ=11.00 (s, 1H), 9.57 (br s, 1H), 8.28 (s, 1H), 8.17 (br s, 1H), 7.78 (s, 1H), 7.72-7.58 (m, 3H), 6.69 (d, J=9.1 Hz, 1H), 5.25 (s, 2H), 5.13 (dd, J=5.1, 13.3 Hz, 1H), 4.50-4.32 (m, 4H), 3.71 (d, J=11.6 Hz, 2H), 2.97-2.89 (m, 1H), 2.88 (d, J=2.4 Hz, 1H), 2.85 (d, J=2.4 Hz, 1H), 2.65-2.58 (m, 1H), 2.46-2.35 (m, 1H), 2.06-1.98 (m, 1H), 1.88-1.78 (m, 2H), 1.77-1.70 (m, 2H). MS (ESI) m/z 506.3 [M+H]⁺

Step 1: To a solution of 2-chloro-5-nitropyridine (1.00 g, 6.31 mmol, 1.00 eq) and 8-oxa-3-azabicyclo[3.2.1]octane hydrochloride (1.00 g, 6.68 mmol, 1.06 eq, hydrochloride) in dimethylformamide (10.0 mL) was added potassium carbonate (2.62 g, 18.9 mmol, 3.00 eq) in portions. The reaction was stirred at 25° C. for 3 h. The mixture was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford 3-(5-nitro-2-pyridyl)-8-oxa-3-azabicyclo[3.2.1]octane.

Step 2: To a solution of 3-(5-nitropyridin-2-yl)-8-oxa-3-azabicyclo[3.2.1]octane (800 mg, 3.40 mmol, 1.00 eq) in methanol (10.0 mL) was added palladium on carbon (10% weight on C) (80.0 mg) in one portion under nitrogen. The reaction was stirred at 25° C. for 1 h under hydrogen (15 Psi). The mixture was filtered, and the filtrate was concentrated under reduced pressure to afford 6-(8-oxa-3-azabicyclo[3.2.1]octan-3-yl)pyridin-3-amine.

Step 3: To a solution of 6-(8-oxa-3-azabicyclo[3.2.1]octan-3-yl)pyridin-3-amine (400 mg, 1.95 mmol, 1.00 eq) and pyridine (0.79 mL, 9.74 mmol, 5.00 eq) in acetonitrile (5.00 mL) was added phenyl chloroformate (0.32 mL, 2.53 mmol, 1.30 eq) at 0° C. The reaction was stirred at 25° C. for 12 h. The mixture was concentrated and the obtained residue was purified by standard methods to afford phenyl (6-(8-oxa-3-azabicyclo[3.2.1]octan-3-yl)pyridin-3-yl)carbamate.

Compound 266: General procedure A with variant i) was used for the preparation from compound VIII employing phenyl (2,3-dihydrobenzofuran-5-yl)carbamate. ¹H NMR (400 MHz, DMSO-d₆) δ=11.00 (s, 1H), 9.56 (br s, 1H), 7.80 (s, 1H), 7.74-7.58 (m, 2H), 7.36 (br s, 1H), 7.14 (br d, J=8.0 Hz, 1H), 6.68 (d, J=8.6 Hz, 1H), 5.26 (s, 2H), 5.14 (dd, J=5.0, 13.4 Hz, 1H), 4.54-4.44 (m, 3H), 4.42-4.30 (m, 1H), 3.18-3.10 (m, 2H), 2.98-2.86 (m, 1H), 2.62 (br d, J=16.8 Hz, 1H), 2.48-2.32 (m, 1H), 2.10-1.98 (m, 1H). MS (ESI) m/z 436.2 [M+H]⁺

To a mixture of 2,3-dihydrobenzofuran-5-amine (400 mg, 2.96 mmol, 1.00 eq) and phenyl chloroformate (0.41 mL, 3.26 mmol, 1.10 eq) in acetonitrile (3.00 mL) was added pyridine (0.72 mL, 8.88 mmol, 3.00 eq) in one portion. The reaction was stirred at 25° C. for 2 h. The mixture was diluted with water (30.0 mL) and exacted with ethyl acetate (3×30.0 mL). The combined organic phases were separated, washed with brine (2×10.0 mL), dried over sodium sulfate, filtered, and concentrated and the obtained residue was purified by standard methods to afford phenyl (2,3-dihydrobenzofuran-5-yl)carbamate.

Compound 267: General procedure A with variant i) was used for the preparation from compound VIII employing phenyl chroman-6-ylcarbamate. ¹H NMR (400 MHz, DMSO-d₆) δ=11.00 (s, 1H), 9.52 (br s, 1H), 7.78 (s, 1H), 7.70-7.58 (m, 2H), 7.17 (br s, 1H), 7.10 (br d, J=8.8 Hz, 1H), 6.64 (d, J=8.8 Hz, 1H), 5.24 (s, 2H), 5.13 (dd, J=5.2, 13.2 Hz, 1H), 4.51-4.42 (m, 1H), 4.39-4.30 (m, 1H), 4.11-4.03 (m, 2H), 2.97-2.86 (m, 1H), 2.69 (t, J=6.4 Hz, 2H), 2.61 (br d, J=17.6 Hz, 1H), 2.41 (dt, J=8.8, 13.2 Hz, 1H), 2.06-1.98 (m, 1H), 1.93-1.84 (m, 2H). MS (ESI) m/z 450.3 [M+H]⁺

Step 1: A solution of chromane (1.00 g, 7.45 mmol, 1.00 eq) in nitric acid (10.0 mL) was stirred at −10° C. for 1 h. The reaction mixture was diluted with ice water (50.0 mL) and then quenched by addition of sodium hydroxide aqueous solution (1 N, 50.0 mL) at 0° C. The mixture was extracted with dichloromethane (3×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=10/1 to 5/1) to afford 6-nitrochroman.

Step 2: To a solution of 6-nitrochromane (650 mg, 3.63 mmol, 1.00 eq) in ethyl acetate (6.00 mL) was added palladium on carbon (10% weight on C) (100 mg) under nitrogen atmosphere. The reaction mixture was stirred at 25° C. for 2 h under hydrogen. The mixture was filtered and concentrated under reduced pressure to afford chroman-6-amine.

Step 3: To a solution of chroman-6-amine (120 mg, 804 μmol, 1.00 eq) and pyridine (0.20 mL, 2.41 mmol, 3.00 eq) in acetonitrile (2.00 mL) was added phenyl chloroformate (0.12 mL, 965 μmol, 1.20 eq) at 25° C. The reaction was stirred at 25° C. for 12 h. The mixture was concentrated and the obtained residue was purified by standard methods to afford phenyl chroman-6-ylcarbamate.

Compound 268: General procedure A with variant i) was used for the preparation from compound VIII employing phenyl (3-chloro-4-methyl-5-((1-methylpyrrolidin-3-yl)oxy)phenyl)carbamate. ¹H NMR (400 MHz, DMSO-d₆) δ=11.70-11.08 (m, 1H), 10.70 (br s, 1H), 9.70 (s, 1H), 7.80 (s, 1H), 7.70-7.66 (m, 1H), 7.66-7.62 (m, 1H), 7.26 (br s, 1H), 7.12 (s, 1H), 5.30 (s, 2H), 5.08 (br dd, J=5.4, 13.0 Hz, 2H), 4.54-4.46 (m, 1H), 4.46-4.36 (m, 1H), 4.06-3.56 (m, 3H), 3.44-3.36 (m, 2H), 2.92-2.84 (m, 4H), 2.68-2.62 (m, 1H), 2.44 (dd, J=4.6, 13.2 Hz, 1H), 2.20 (s, 4H), 2.12-2.04 (m, 1H). MS (ESI) m/z 541.3 [M+H]⁺

Step 1: A solution of tert-butyl 3-(3-chloro-2-methyl-5-nitrophenoxy)pyrrolidine-1-carboxylate (described in example 44) (730 mg, 2.05 μmol, 1.00 eq) and hydrochloric acid/ethyl acetate (10.0 mL) in ethyl acetate (20.0 mL) was stirred at 25° C. for 4 h. The mixture was concentrated under reduced pressure to give a residue. The residue was diluted with saturated sodium bicarbonate (50.0 mL) and extracted with ethyl acetate (3×20.0 mL). The combined organic layers were washed with brine (20.0 mL), dried over anhydrous sodium sulfate, filtered, and concentrated under reduced pressure to afford 3-(3-chloro-2-methyl-5-nitrophenoxy)pyrrolidine.

Step 2: To a solution of 3-(3-chloro-2-methyl-5-nitrophenoxy)pyrrolidine (400 mg, 1.56 mmol, 1.00 eq) and formaldehyde 37% (12.0 mL, 161 mmol, 103 eq) in methanol (3.00 mL) was added acetic acid (0.09 mL, 1.56 mmol, 1.00 eq) at 25° C. The reaction was stirred at 25° C. for 0.5 h. Then sodium cyanoborohydride (979 mg, 15.6 mmol, 10.0 eq) was added, and the reaction was stirred at 25° C. for another 1.5 h. The mixture was concentrated under reduced pressure to give a residue. The residue was diluted with water (50.0 mL) and extracted with ethyl acetate (3×20.0 mL). The combined organic layers were washed with brine (20.0 mL), dried over sodium sulfate, filtered, and concentrated under reduced pressure to give a residue. The residue was purified by reversed phase preparative HPLC to afford 3-(3-chloro-2-methyl-5-nitrophenoxy)-1-methylpyrrolidine.

Step 3: To a mixture of 3-(3-chloro-2-methyl-5-nitrophenoxy)-1-methylpyrrolidine (250 mg, 923 μmol, 1.00 eq), iron powder (258 mg, 4.62 mmol, 5.00 eq) and ammonium chloride (247 g, 4.62 mmol, 5.00 eq) in methanol (5.00 mL) was added water (5.00 mL) at 25° C. The reaction was stirred at 80° C. for 2 h. The mixture was concentrated under reduced pressure to give a residue. The residue was poured into saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-4-methyl-5-((1-methylpyrrolidin-3-yl)oxy)aniline.

Step 4: To a solution of 3-chloro-4-methyl-5-(1-methylpyrrolidin-3-yl)oxy-aniline (116 mg, 482 μmol, 1.00 eq) and phenyl chloroformate (0.66 mL, 530 μmol, 1.10 eq) in acetonitrile (5.00 mL) was added pyridine (0.12 mL, 1.45 mmol, 3.00 eq) in one portion at 25° C. The reaction was stirred at 25° C. for 2 h. The mixture was diluted with water (30.0 mL) and exacted with ethyl acetate (3×30.0 mL). The combined organic layers were washed with brine (2×10.0 mL), dried over sodium sulfate, filtered, and concentrated and the obtained residue was purified by standard methods to afford (3-chloro-4-methyl-5-((1-methylpyrrolidin-3-yl)oxy)phenyl)carbamate.

Compound 269: General procedure A with variant i) was used for the preparation from compound VIII employing phenyl (6-(3-methylpyrrolidin-1-yl)pyridin-3-yl)carbamate. ¹H NMR (400 MHz, DMSO-d₆) δ=11.00 (s, 1H), 9.42 (br s, 1H), 8.15 (s, 1H), 8.10 (br s, 1H), 7.78 (s, 1H), 7.70-7.62 (m, 2H), 7.58 (br d, J=7.7 Hz, 1H), 6.38 (d, J=8.9 Hz, 1H), 5.24 (s, 2H), 5.13 (dd, J=5.1, 13.3 Hz, 1H), 4.52-4.43 (m, 1H), 4.39-4.31 (m, 1H), 3.54 (dd, J=7.3, 9.9 Hz, 1H), 3.47-3.43 (m, 1H), 3.29-3.27 (m, 1H), 2.98-2.85 (m, 2H), 2.65-2.56 (m, 1H), 2.47-2.38 (m, 1H), 2.36-2.29 (m, 1H), 2.12-1.95 (m, 2H), 1.55 (qd, J=8.3, 12.1 Hz, 1H), 1.07 (d, J=6.6 Hz, 3H). MS (ESI) m/z 478.2 [M+H]⁺

Step 1: To a solution of 2-chloro-5-nitropyridine (2.00 g, 12.6 mmol, 1.00 eq) in dimethylformamide (20.0 mL) was added 3-methylpyrrolidine hydrochloride (2.30 g, 18.9 mmol, 1.50 eq) and potassium carbonate (5.23 g, 37.8 mmol, 3.00 eq). The reaction was stirred at 60° C. for 1 h. The mixture was poured into water (500 ml) at 0° C. The resulting precipitate was collected by filtration and dried under vacuum to afford 2-(3-methylpyrrolidin-1-yl)-5-nitropyridine.

Step 2: To a solution of 2-(3-methylpyrrolidin-1-yl)-5-nitropyridine (2.60 g, 12.5 mmol, 1.00 eq) in methanol (30.0 mL) was added palladium on carbon (10% weight on C) (50.0 mg). The reaction was stirred at 25° C. for 1 h under hydrogen (15 Psi). The mixture was filtered, and the filtrate was concentrated under reduced pressure to afford 6-(3-methylpyrrolidin-1-yl)pyridin-3-amine.

Step 3: To a solution of 6-(3-methylpyrrolidin-1-yl)pyridin-3-amine (1.60 g, 9.03 mmol, 1.00 eq) and pyridine (3.64 mL, 45.1 mmol, 5.00 eq) in acetonitrile (16.0 mL) was added phenyl chloroformate (1.47 mL, 11.7 mmol, 1.30 eq). The reaction was stirred at 25° C. for 1 h. The mixture was concentrated and the residue was purified by a standard method to afford phenyl(6-(3-methylpyrrolidin-1-yl)pyridin-3-yl)carbamate.

Compound 270: General procedure A with variant i) was used for the preparation from compound VIII employing phenyl (3-chloro-4-methyl-5-((1-methylpyrrolidin-3-yl)methoxy)phenyl)carbamate. ¹H NMR (400 MHz, DMSO-d₆) δ=11.00 (s, 1H), 10.85 (br d, J=3.7 Hz, 1H), 9.95 (s, 1H), 7.79 (s, 1H), 7.73-7.60 (m, 2H), 7.26-7.10 (m, 2H), 5.28 (s, 2H), 5.13 (dd, J=5.1, 13.3 Hz, 1H), 4.54-4.44 (m, 1H), 4.41-4.29 (m, 1H), 4.08-3.93 (m, 2H), 3.75-3.69 (m, 1H), 3.49-3.41 (m, 1H), 3.26-3.16 (m, 1H), 3.13-3.01 (m, 1H), 2.97-2.86 (m, 2H), 2.81 (t, J=4.8 Hz, 3H), 2.61 (br d, J=17.9 Hz, 1H), 2.56-2.52 (m, 1H), 2.41 (dq, J=4.4, 13.2 Hz, 1H), 2.32-2.15 (m, 1H), 2.14 (s, 3H), 2.06-1.97 (m, 1H), 1.96-1.71 (m, 1H). MS (ESI) m/z 555.2 [M+H]⁺

Step 1: A mixture of tert-butyl 3-((3-chloro-2-methyl-5-nitrophenoxy)methyl)pyrrolidine-1-carboxylate (described in example 52) (1.26 g, 3.40 mmol, 1.00 eq) in hydrochloric acid/ethyl acetate (4.0 M, 1.13 mL) was stirred at 25° C. for 1 h. The mixture was concentrated under reduced pressure to afford 3-((3-chloro-2-methyl-5-nitrophenoxy)methyl)pyrrolidine.

Step 2: To a solution of 3-((3-chloro-2-methyl-5-nitrophenoxy)methyl)pyrrolidine (900 mg, 3.32 mmol, 1.00 eq) in 2,2,2-trifluoroethanol (10.0 mL) was added paraformaldehyde (0.46 mL, 16.6 mmol, 5.00 eq). The reaction was stirred at 60° C. for 0.5 h. Then sodium borohydride (252 mg, 6.65 mmol, 2.00 eq) was added in portions, and the reaction was stirred at 60° C. for 1 h. The mixture was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford 3-((3-chloro-2-methyl-5-nitrophenoxy)methyl)-1-methylpyrrolidine.

Step 3: To a solution of 3-((3-chloro-2-methyl-5-nitrophenoxy)methyl)-1-methylpyrrolidine (850 mg, 2.99 mmol, 1.00 eq) and ammonium chloride (798 mg, 14.9 mmol, 5.00 eq) in methanol (5.00 mL) and water (5.00 mL) was added iron powder (500 mg, 8.96 mmol, 3.00 eq) in portions. The reaction was stirred at 80° C. for 2 h. The mixture was concentrated under reduced pressure to give a residue. The residue was purified by reversed phase preparative HPLC to afford 3-chloro-4-methyl-5-((1-methylpyrrolidin-3-yl)methoxy)aniline.

Step 4: To a solution of 3-chloro-4-methyl-5-((1-methylpyrrolidin-3-yl)methoxy)aniline (300 mg, 1.18 mmol, 1.00 eq) and pyridine (0.29 mL, 3.53 mmol, 3.00 eq) in acetonitrile (5.00 mL) was added phenyl chloroformate (0.22 mL, 1.77 mmol, 1.50 eq) dropwise at 0° C. The reaction was stirred at 25° C. for 12 h. The mixture was concentrated and the obtained residue was purified by standard methods to afford phenyl (3-chloro-4-methyl-5-((1-methylpyrrolidin-3-yl) methoxy)phenyl)carbamate.

Compound 271: General procedure A with variant i) was used for the preparation from compound VIII employing phenyl (2-ethyl-6-methylpyridin-4-yl)carbamate. ¹H NMR (400 MHz, DMSO-d₆) δ=11.00 (br s, 1H), 10.11 (s, 1H), 8.27 (br s, 1H), 7.80 (s, 1H), 7.72-7.59 (m, 2H), 7.14 (d, J=3.5 Hz, 2H), 5.30 (s, 2H), 5.13 (dd, J=5.1, 13.3 Hz, 1H), 4.51-4.31 (m, 2H), 2.98-2.87 (m, 1H), 2.61 (q, J=7.5 Hz, 3H), 2.41 (br dd, J=4.4, 13.1 Hz, 1H), 2.35 (s, 3H), 2.07-1.96 (m, 1H), 1.17 (t, J=7.6 Hz, 3H). MS (ESI) m/z 437.1 [M+H]⁺

Step 1: To a mixture of 2-chloro-6-methyl-4-nitropyridine (2.00 g, 11.6 mmol, 1.00 eq), ethylboronic acid (2.57 g, 34.8 mmol, 3.00 eq) and potassium carbonate (4.81 g, 34.8 mmol, 3.00 eq) in dioxane (20.0 mL) was added tetrakis(triphenylphosphine)palladium(0) (1.34 g, 1.16 mmol, 0.100 eq) under nitrogen atmosphere. The reaction was stirred at 110° C. for 12 h. The mixture was concentrated under reduced pressure to give a residue. The residue was purified by silica gel column chromatography (petroleum ether/ethyl acetate=10/1) to afford 2-ethyl-6-methyl-4-nitropyridine.

Step 2: To a solution of 2-ethyl-6-methyl-4-nitropyridine (650 mg, 3.91 mmol, 1.00 eq) in methanol (50.0 mL) was added palladium on carbon (10% weight on C) (10.0 mg) in one portion. The reaction was stirred at 25° C. for 2 h under hydrogen (15 Psi). The mixture was filtered, and the filtrate was concentrated under reduced pressure to afford 2-ethyl-6-methylpyridin-4-amine.

Step 3: To a solution of 2-ethyl-6-methylpyridin-4-amine (450 mg, 3.30 mmol, 1.00 eq) in dimethylformamide (5.00 mL) was added sodium hydride (60% dispersion in mineral oil) (396 mg, 9.91 mmol, 3.00 eq) in portions at 0° C. The reaction was stirred at 0° C. for 0.5 h. Then phenyl chloroformate (0.50 mL, 3.96 mmol, 1.20 eq) was added dropwise at 0° C. The reaction was stirred at 25° C. for 12 h, then it was quenched with 1 M hydrochloric acid and filtered. The filtrate was concentrated and the obtained residue was purified by standard methods to afford phenyl (2-ethyl-6-methylpyridin-4-yl)carbamate.

Compound 272: General procedure A with variant i) was used for the preparation from compound VIII employing phenyl (5-fluoro-6-(piperidin-1-yl)pyridin-3-yl)carbamate. ¹H NMR (400 MHz, DMSO-d₆) δ=11.00 (s, 1H), 9.92 (br s, 1H), 8.07 (s, 1H), 7.79 (s, 1H), 7.72-7.59 (m, 3H), 5.28 (s, 2H), 5.13 (dd, J=5.1, 13.3 Hz, 1H), 4.52-4.43 (m, 1H), 4.38-4.31 (m, 1H), 3.23 (br d, J=5.5 Hz, 4H), 2.97-2.87 (m, 1H), 2.65-2.57 (m, 1H), 2.46-2.35 (m, 1H), 2.07-1.97 (m, 1H), 1.66-1.54 (m, 6H). MS (ESI) m/z 496.2 [M+H]⁺

Step 1: To a solution of 2-chloro-3-fluoro-5-nitropyridine (0.500 g, 2.83 mmol, 1.00 eq) in dimethylformamide (5.00 mL) were added piperidine (250 mg, 2.94 mmol, 1.04 eq) and potassium carbonate (800 mg, 5.79 mmol, 2.04 eq). The reaction was stirred at 25° C. for 2 h. The mixture was poured into water (100 mL). The resulting precipitate was collected by filtration and dried under vacuum to afford 3-fluoro-5-nitro-2-(piperidin-1-yl)pyridine.

Step 2: To a solution of 3-fluoro-5-nitro-2-(piperidin-1-yl)pyridine (515 mg, 2.29 mmol, 1.00 eq) in methanol (10.0 mL) was added wet palladium on carbon (10% weight on C) (50.0 mg) under hydrogen. The mixture was stirred at 25° C. for 2 h under hydrogen (15.0 Psi). The mixture was filtered, and the filtrate was concentrated under reduced pressure to afford 5-fluoro-6-(piperidin-1-yl)pyridin-3-amine.

Step 3: To a solution of 5-fluoro-6-(piperidin-1-yl)pyridin-3-amine (440 mg, 2.25 mmol, 1.00 eq) in acetonitrile (10.0 mL) were added pyridine (0.11 mL, 11.3 mmol, 5.00 eq) and phenyl chloroformate (0.34 mL, 2.70 mmol, 1.20 eq) at 0° C. The reaction was stirred at 0° C. for 1 h. The pH was adjusted to pH=5 with formic acid (1.00 mL) and the solution was filtered. The filtrate was concentrated and the obtained residue was purified by standard methods to afford phenyl (5-fluoro-6-(piperidin-1-yl)pyridin-3-yl)carbamate.

Compound 273: General procedure A with variant i) was used for the preparation from compound VIII employing phenyl (6-(3,3-difluoropiperidin-1-yl)pyridin-3-yl)carbamate. ¹H NMR (400 MHz, DMSO-d₆) δ=11.00 (br s, 1H), 9.61 (br s, 1H), 8.32 (br s, 1H), 8.18 (br s, 1H), 7.79 (s, 1H), 7.72-7.60 (m, 3H), 6.91 (d, J=9.1 Hz, 1H), 5.26 (s, 2H), 5.13 (dd, J=5.1, 13.3 Hz, 1H), 4.52-4.44 (m, 1H), 4.39-4.30 (m, 1H), 3.80 (t, J=12.1 Hz, 2H), 3.51-3.47 (m, 2H), 2.98-2.86 (m, 1H), 2.64-2.58 (m, 1H), 2.47-2.36 (m, 1H), 2.11-1.99 (m, 3H), 1.77-1.69 (m, 2H). MS (ESI) m/z 514.2 [M+H]⁺

Step 1: To a solution of 2-chloro-5-nitropyridine (300 mg, 1.89 mmol, 1.00 eq) in dimethylformamide (6.00 mL) were added potassium carbonate (784 mg, 5.68 mmol, 3.00 eq) and 3,3-difluoropiperidine hydrochloride (447 mg, 2.84 mmol, 1.50 eq). The reaction was stirred at 50° C. for 2 h. The mixture was poured slowly into ice-water (40.0 mL). The resulting precipitate was collected by filtration and dried under vacuum to afford 2-(3,3-difluoropiperidin-1-yl)-5-nitropyridine.

Step 2: To a solution of 2-(3,3-difluoropiperidin-1-yl)-5-nitropyridine (452 mg, 1.86 mmol, 1.00 eq) in methanol (10.0 mL) was added wet palladium on carbon (10% weight on C) (50.0 mg) under hydrogen. The reaction was stirred at 25° C. for 3 h under hydrogen (15 Psi). The mixture was filtered, and the filtrate was concentrated under reduced pressure to afford 6-(3,3-difluoropiperidin-1-yl)pyridin-3-amine.

Step 3: To a solution of 6-(3,3-difluoropiperidin-1-yl)pyridin-3-amine (380 mg, 1.78 mmol, 1.00 eq) and pyridine (0.72 mL, 8.91 mmol, 5.00 eq) in acetonitrile (10.0 mL) was added phenyl chloroformate (0.34 mL, 2.67 mmol, 1.50 eq) at 0° C. The reaction was stirred at 0° C. for 1 h. The mixture was concentrated and the obtained residue was purified by standard methods to afford phenyl (6-(3,3-difluoropiperidin-1-yl)pyridin-3-yl)carbamate.

Compound 274: General procedure A with variant i) was used for the preparation from compound VIII employing phenyl (6-(2-methylpyrrolidin-1-yl)pyridin-3-yl)carbamate. ¹H NMR (400 MHz, DMSO-d₆) δ=11.00 (s, 1H), 9.42 (br s, 1H), 8.14 (s, 1H), 8.10 (br s, 1H), 7.78 (s, 1H), 7.65 (q, J=7.9 Hz, 2H), 7.57 (br d, J=7.8 Hz, 1H), 6.41 (d, J=9.1 Hz, 1H), 5.24 (s, 2H), 5.13 (dd, J=5.1, 13.3 Hz, 1H), 4.53-4.43 (m, 1H), 4.39-4.29 (m, 1H), 4.11-4.00 (m, 1H), 3.43 (ddd, J=2.4, 7.4, 9.8 Hz, 1H), 3.26-3.15 (m, 1H), 2.99-2.86 (m, 1H), 2.61 (br d, J=18.5 Hz, 1H), 2.41 (br dd, J=4.5, 13.1 Hz, 1H), 2.07-1.96 (m, 3H), 1.95-1.87 (m, 1H), 1.65 (br dd, J=2.9, 4.6 Hz, 1H), 1.13 (d, J=6.1 Hz, 3H). MS (ESI) m/z 478.3 [M+H]⁺

Step 1: To a solution of 2-chloro-5-nitropyridine (400 mg, 2.52 mmol, 1.00 eq) in dimethylformamide (6.00 mL) were added potassium carbonate (1.05 g, 7.57 mmol, 3.00 eq) and 2-methylpyrrolidine hydrochloride (460 mg, 3.78 mmol, 1.50 eq). The reaction was stirred at 50° C. for 1 h. The mixture was slowly poured into ice-water (40.0 ml). The resulting precipitate was collected by filtration and dried under vacuum to afford 2-(2-methylpyrrolidin-1-yl)-5-nitropyridine.

Step 2: To a solution of 2-(2-methylpyrrolidin-1-yl)-5-nitropyridine (512 mg, 2.47 mmol, 1.00 eq) in methanol (10.0 mL) was added wet palladium on carbon (10% weight on C) (50.0 mg) under hydrogen. The reaction was stirred at 25° C. for 3 h under hydrogen (15.0 psi). The mixture was filtered, and the filtrate was concentrated under reduced pressure to afford 6-(2-methylpyrrolidin-1-yl)pyridin-3-amine.

Step 3: To a solution of 6-(2-methylpyrrolidin-1-yl)pyridin-3-amine (371 mg, 2.09 mmol, 1.00 eq) and pyridine (0.85 mL, 10.5 mmol, 5.00 eq) in acetonitrile (10.0 mL) was added phenyl chloroformate (0.39 mL, 3.14 mmol, 1.50 eq) at 0° C. The reaction was stirred at 0° C. for 1 h. The mixture was concentrated and the obtained residue was purified by standard methods to afford phenyl (6-(2-methylpyrrolidin-1-yl)pyridine-3-yl)carbamate.

Compound 275: General procedure A with variant i) was used for the preparation from compound VIII employing phenyl (6-(piperidin-1-yl)pyridazin-3-yl)carbamate. ¹H NMR (400 MHz, DMSO-d₆) δ=11.08 (s, 1H), 11.00 (s, 1H), 8.24-8.15 (m, 1H), 8.13-8.04 (m, 1H), 7.82 (s, 1H), 7.76-7.60 (m, 2H), 5.34 (s, 2H), 5.13 (dd, J=5.1, 13.3 Hz, 1H), 4.52-4.45 (m, 1H), 4.39-4.33 (m, 1H), 3.70 (br s, 4H), 3.01-2.84 (m, 1H), 2.61 (br d, J=17.9 Hz, 1H), 2.45-2.36 (m, 1H), 2.07-1.97 (m, 1H), 1.65 (br s, 6H). MS (ESI) m/z 479.2 [M+H]⁺

Step 1: To a solution of 6-chloropyridazin-3-amine (500 mg, 3.86 mmol, 1.00 eq) and piperidine (0.76 mL, 7.72 mmol, 2.00 eq) in dimethylformamide (3.00 mL) was added N,N-diisopropylethylamine (1.34 mL, 7.72 mmol, 2.00 eq) dropwise. The reaction was stirred at 180° C. for 2 h under microwave irradiation. The mixture was filtered, and the filtrate was concentrated under reduced pressure to give a residue. The residue was purified by reversed phase preparative HPLC to afford 6-(piperidin-1-yl)pyridazin-3-amine.

Step 2: To a solution of 6-(piperidin-1-yl)pyridazin-3-amine (180 mg, 1.01 mmol, 1.00 eq) and pyridine (0.25 mL, 3.03 mmol, 3.00 eq) in dimethylformamide (0.50 mL) and acetonitrile (3.00 mL) was added phenyl chloroformate (0.16 mL, 1.31 mmol, 1.30 eq) dropwise at 0° C. The reaction was stirred at 25° C. for 12 h. The mixture was concentrated and the obtained residue was purified by standard methods to afford phenyl (6-(piperidin-1-yl)pyridazin-3-yl)carbamate.

Compound 276: General procedure A with variant i) was used for the preparation from compound VIII employing phenyl (6-morpholinopyridin-3-yl)carbamate. ¹H NMR (400 MHz, DMSO-d₆) δ=10.99 (br s, 1H), 9.61 (br s, 1H), 8.20 (br s, 1H), 7.78 (s, 1H), 7.70-7.61 (m, 3H), 6.81 (d, J=9.0 Hz, 1H), 5.25 (s, 2H), 5.12 (dd, J=5.1, 13.4 Hz, 1H), 4.51-4.43 (m, 1H), 4.37-4.30 (m, 1H), 3.70-3.66 (m, 4H), 3.35 (br s, 4H), 2.97-2.86 (m, 1H), 2.63-2.57 (m, 1H), 2.40 (br dd, J=4.6, 13.2 Hz, 1H), 2.05-1.97 (m, 1H). MS (ESI) m/z 480.2 [M+H]⁺

To a solution of 6-morpholinopyridin-3-amine (300 mg, 1.67 mmol, 1.00 eq) and pyridine (0.41 mL, 5.02 mmol, 3.00 eq) in acetonitrile (8.00 mL) was added phenyl chloroformate (0.25 mL, 2.01 mmol, 1.20 eq) in portions at 0° C. The reaction was stirred at 25° C. for 1 h. The mixture was concentrated under reduced pressure to give a residue which was diluted with ethyl acetate (6.00 mL) and water (4.00 mL). The resulting precipitate was collected by filtration and dried by standard methods to afford phenyl (6-morpholinopyridin-3-yl)carbamate.

Compound 277: General procedure A with variant i) was used for the preparation from compound VIII employing phenyl (6-(2-methylpiperidin-1-yl)pyridin-3-yl)carbamate. ¹H NMR (400 MHz, DMSO-d₆) δ=11.07-10.89 (m, 1H), 9.50 (br s, 1H), 8.14 (br s, 1H), 7.78 (s, 1H), 7.72-7.51 (m, 3H), 6.72 (d, J=9.3 Hz, 1H), 5.25 (s, 2H), 5.13 (dd, J=5.0, 13.3 Hz, 1H), 4.59-4.51 (m, 1H), 4.51-4.43 (m, 1H), 4.40-4.29 (m, 1H), 3.97 (br d, J=13.0 Hz, 1H), 2.99-2.86 (m, 1H), 2.79 (dt, J=2.8, 12.8 Hz, 1H), 2.65-2.57 (m, 1H), 2.48-2.38 (m, 1H), 2.07-1.96 (m, 1H), 1.73-1.54 (m, 5H), 1.47-1.31 (m, 1H), 1.02 (d, J=6.7 Hz, 3H). MS (ESI) m/z 492.2 [M+H]⁺

Step 1: To a solution of 2-chloro-5-nitropyridine (2.00 g, 12.6 mmol, 1.00 eq) in dimethylformamide (10.0 mL) were added 2-methylpiperidine (2.24 mL, 18.9 mmol, 1.50 eq) and potassium carbonate (5.23 g, 37.8 mmol, 3.00 eq). The reaction was stirred at 60° C. for 1 h. The mixture was poured into water (200 mL) and extracted with ethyl acetate (2×100 mL). The combined organic layers were dried over sodium sulfate, filtered, and concentrated under reduced pressure to afford 2-(2-methylpiperidin-1-yl)-5-nitropyridine.

Step 2: To a solution of 2-(2-methylpiperidin-1-yl)-5-nitropyridine (2.70 g, 12.2 mmol, 1.00 eq) in methanol (30.0 mL) was added palladium on carbon (50.0 mg, 10% weight on C). The reaction was stirred at 25° C. for 1 h under hydrogen (15 Psi). The mixture was filtered, and the filtrate was concentrated under reduced pressure to afford 6-(2-methylpiperidin-1-yl)pyridin-3-amine.

Step 3: To a solution of 6-(2-methylpiperidin-1-yl)pyridin-3-amine (2.20 g, 11.5 mmol, 1.00 eq) and pyridine (4.64 mL, 57.5 mmol, 5.00 eq) in acetonitrile (30.0 mL) was added phenyl chloroformate (1.73 mL, 13.8 mmol, 1.20 eq). The reaction was stirred at 25° C. for 1 h. The mixture was concentrated and the obtained residue was purified by standard methods to afford phenyl (6-(2-methylpiperidin-1-yl) pyridin-3-yl)carbamate.

Compound 278: General procedure A with variant i) was used for the preparation from compound VIII employing phenyl (6-(4,4-difluoropiperidin-1-yl)pyridin-3-yl) carbamate. ¹H NMR (400 MHz, DMSO-d₆) δ=11.00 (s, 1H), 9.63 (br s, 1H), 8.30-8.11 (m, 1H), 7.79 (s, 1H), 7.75-7.60 (m, 3H), 6.95 (d, J=9.2 Hz, 1H), 5.26 (s, 2H), 5.13 (dd, J=5.1, 13.3 Hz, 1H), 4.52-4.43 (m, 1H), 4.40-4.30 (m, 1H), 3.67-3.58 (m, 4H), 2.99-2.85 (m, 1H), 2.65-2.57 (m, 1H), 2.46-2.36 (m, 1H), 2.05-1.91 (m, 5H). MS (ESI) m/z 514.2 [M+H]⁺

Step 1: To a solution of 2-chloro-5-nitropyridine (2.00 g, 12.6 mmol, 1.00 eq) in dimethylformamide (10.0 mL) was added 4,4-difluoropiperidine hydrochloride (2.00 g, 12.6 mmol, 1.01 eq, HCl) and potassium carbonate (4.00 g, 28.9 mmol, 2.29 eq). The reaction was stirred at 60° C. for 2 h. The mixture was poured into water (100 mL). The resulting yellow precipitate was collected by filtration and dried under vacuum to afford 2-(4,4-difluoropiperidin-1-yl)-5-nitropyridine.

Step 2: To a solution of 2-(4,4-difluoropiperidin-1-yl)-5-nitropyridine (2.70 g, 11.1 mmol, 1.00 eq) in methanol (30.0 mL) was added palladium on carbon (50.0 mg, 10% weight on C). The reaction was stirred at 25° C. for 1 h under hydrogen (15 Psi). The mixture was filtered, and the filtrate was concentrated under reduced pressure to afford 6-(4,4-difluoropiperidin-1-yl)pyridin-3-amine.

Step 3: To a solution of 6-(4,4-difluoropiperidin-1-yl)pyridin-3-amine (2.30 g, 10.7 mmol, 1.00 eq) and pyridine (4.35 mL, 53.9 mmol, 5.00 eq) in acetonitrile (30.0 mL) was added phenyl chloroformate (1.62 mL, 12.9 mmol, 1.20 eq). The reaction was stirred at 25° C. for 1 h. The mixture was concentrated under reduced pressure to give a residue. Water (30.0 mL) was added, and the mixture was exacted with ethyl acetate (3×30.0 mL). The combined organic layers were dried, filtered, and concentrated by standard methods to afford phenyl(6-(4,4-difluoropiperidin-1-yl) pyridin-3-yl)carbamate.

Compound 279: General procedure A with variant i) was used for the preparation from compound VIII employing (2,2-difluorobenzo[d][1,3]dioxol-5-yl)carbamate. ¹H NMR (400 MHz, DMSO-d₆) δ=10.99 (br s, 1H), 10.06 (br s, 1H), 7.80 (s, 1H), 7.71-7.67 (m, 1H), 7.67-7.62 (m, 1H), 7.58 (d, J=1.6 Hz, 1H), 7.33 (d, J=8.8 Hz, 1H), 7.18 (dd, J=2.0, 8.8 Hz, 1H), 5.29 (s, 2H), 5.13 (dd, J=5.2, 13.2 Hz, 1H), 4.54-4.43 (m, 1H), 4.41-4.29 (m, 1H), 2.98-2.86 (m, 1H), 2.64-2.57 (m, 1H), 2.46-2.35 (m, 1H), 2.06-1.97 (m, 1H). MS (ESI) m/z 474.2 [M+H]⁺

To a solution of 2,2-difluorobenzo[d][1,3]dioxol-5-amine (500 mg, 2.57 mmol, 1.00 eq) and pyridine (0.70 mL, 8.66 mmol, 3.00 eq) in acetonitrile (5.00 mL) was added phenyl chloroformate (0.43 mL, 3.47 mmol, 1.20 eq) at 25° C. The reaction was stirred at 25° C. for 2 h. The mixture was concentrated and the obtained residue was purified by standard methods to afford phenyl (2,2-difluorobenzo[d][1,3]dioxol-5-yl)carbamate.

Compound 280: General procedure A with variant i) was used for the preparation from compound VIII employing phenyl (6-cyclopropylpyridin-3-yl)carbamate. ¹H NMR (400 MHz, DMSO-d₆) δ=10.99 (br s, 1H), 9.87 (br s, 1H), 8.44 (d, J=2.2 Hz, 1H), 8.30 (s, 1H), 7.79 (s, 1H), 7.74 (br d, J=7.6 Hz, 1H), 7.70-7.66 (m, 1H), 7.65-7.61 (m, 1H), 7.20 (d, J=8.6 Hz, 1H), 5.27 (s, 2H), 5.18-5.05 (m, 1H), 4.51-4.43 (m, 1H), 4.38-4.30 (m, 1H), 2.98-2.84 (m, 1H), 2.65-2.56 (m, 1H), 2.47-2.34 (m, 1H), 2.08-1.95 (m, 2H), 0.92-0.85 (m, 2H), 0.85-0.80 (m, 2H). MS (ESI) m/z 435.1 [M+H]⁺

To a solution of 6-cyclopropylpyridin-3-amine (0.500 g, 3.73 mmol, 1.00 eq) and pyridine (0.90 mL, 11.2 mmol, 3.00 eq) in acetonitrile (5.00 mL) was added phenyl chloroformate (0.56 mL, 4.47 mmol, 1.20 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (6-cyclopropylpyridin-3-yl)carbamate.

Compound 281: General procedure A with variant i) was used for the preparation from compound VIII employing phenyl(4-(oxetan-3-yl)phenyl)carbamate. ¹H NMR (400 MHz, DMSO-d₆) δ=11.00 (br s, 1H), 9.82 (br s, 1H), 7.80 (s, 1H), 7.72-7.66 (m, 1H), 7.66-7.62 (m, 1H), 7.47 (br d, J=8.4 Hz, 2H), 7.32 (d, J=8.6 Hz, 2H), 5.28 (s, 2H), 5.20-5.07 (m, 1H), 4.97-4.85 (m, 2H), 4.58 (t, J=6.4 Hz, 2H), 4.51-4.44 (m, 1H), 4.39-4.30 (m, 1H), 4.24-4.14 (m, 1H), 2.97-2.87 (m, 1H), 2.65-2.57 (m, 1H), 2.48-2.34 (m, 1H), 2.06-1.96 (m, 1H). MS (ESI) m/z 450.2 [M+H]⁺

To a solution of 4-(oxetan-3-yl)aniline (200 mg, 1.34 mmol, 1.00 eq) and pyridine (0.32 mL, 4.02 mmol, 3.00 eq) in acetonitrile (10.0 mL) was added phenyl chloroformate (0.20 mL, 1.61 mmol, 1.20 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl(4-(oxetan-3-yl)phenyl)carbamate.

Compound 282: General procedure A with variant i) was used for the preparation from compound VIII employing phenyl (6-(4-methylpiperazin-1-yl)pyridin-3-yl)carbamate. ¹H NMR (400 MHz, DMSO-d₆) δ=10.99 (s, 1H), 9.57 (br s, 1H), 8.18 (s, 2H), 7.78 (s, 1H), 7.65 (q, J=7.8 Hz, 3H), 6.81 (d, J=9.2 Hz, 1H), 5.24 (s, 2H), 5.12 (dd, J=5.1, 13.3 Hz, 1H), 4.51-4.44 (m, 1H), 4.37-4.30 (m, 1H), 3.42-3.37 (m, 4H), 2.91 (ddd, J=5.3, 13.5, 17.5 Hz, 1H), 2.60 (br dd, J=2.0, 15.6 Hz, 1H), 2.46-2.42 (m, 4H), 2.38 (br d, J=4.4 Hz, 1H), 2.24 (s, 3H), 2.05-1.98 (m, 1H). MS (ESI) m/z 493.3 [M+H]⁺

To a solution of 6-(4-methylpiperazin-1-yl)pyridin-3-amine (500 mg, 2.60 mmol, 1.00 eq) and pyridine (1.05 mL, 13.0 mmol, 5.00 eq) in acetonitrile (5.00 mL) was added phenyl chloroformate (0.39 mL, 3.12 mmol, 1.20 eq) at 0° C. The reaction was stirred at 25° C. for 12 h. The mixture was concentrated and the obtained residue was purified by standard methods to afford phenyl (6-(4-methylpiperazin-1-yl)pyridin-3-yl)carbamate.

Compound 283: General procedure A with variant i) was used for the preparation from compound VIII employing phenyl (3,3-dimethyl-2,3-dihydrobenzofuran-6-yl)carbamate. 1H NMR (400 MHz, DMSO-d₆) δ=11.00 (s, 1H), 9.74 (br s, 1H), 7.80 (s, 1H), 7.74-7.60 (m, 2H), 7.08 (d, J=8.0 Hz, 1H), 6.98 (s, 1H), 6.96-6.84 (m, 1H), 5.26 (s, 2H), 5.14 (dd, J=5.0, 13.4 Hz, 1H), 4.48 (d, J=17.4 Hz, 1H), 4.36 (d, J=17.4 Hz, 1H), 4.20 (s, 2H), 3.00-2.85 (m, 1H), 2.70-2.56 (m, 1H), 2.47-2.33 (m, 1H), 2.12-1.98 (m, 1H), 1.25 (s, 6H). MS (ESI) m/z 464.2 [M+H]⁺

Step 1: To a solution of 2-bromo-5-nitrophenol (1.00 g, 4.59 mmol, 1.00 eq) and 3-bromo-2-methylprop-1-ene (0.60 mL, 5.96 mmol, 1.30 eq) in acetone (5.00 mL) was added potassium carbonate (1.27 g, 9.17 mmol, 2.00 eq) in one portion at 25° C. The reaction was stirred for 12 h at 25° C. The mixture was diluted with water (30.0 mL) and exacted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 1-bromo-2-((2-methylallyl)oxy)-4-nitrobenzene.

Step 2: To a solution of 1-bromo-2-((2-methylallyl)oxy)-4-nitrobenzene (900 mg, 3.31 mmol, 1.00 eq) in dimethylformamide (5.00 mL) were added sodium acetate (678 mg, 8.27 mmol, 2.50 eq), palladium acetate (149 mg, 662 μmol, 0.20 eq), tetraethylammonium iodide (936 mg, 3.64 mmol, 1.10 eq) and sodium formate (0.18 mL, 3.31 mmol, 1.00 eq) in one portion under nitrogen. The reaction was stirred for 12 h at 100° C. The mixture was diluted with water (30.0 mL) and extracted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 3,3-dimethyl-6-nitro-2H-benzofuran.

Step 3: To a solution of 3,3-dimethyl-6-nitro-2,3-dihydrobenzofuran (300 mg, 1.55 mmol, 1.00 eq) in water (3.00 mL) and methanol (6.00 mL) were added ammonium chloride (415 mg, 7.76 mmol, 5.00 eq) and iron powder (434 mg, 7.76 mmol, 5.00 eq). The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 3,3-dimethyl-2,3-dihydrobenzofuran-6-amine.

Step 4: To a solution of 3,3-dimethyl-2,3-dihydrobenzofuran-6-amine (162 mg, 993 μmol, 1.00 eq) and pyridine (0.24 mL, 2.98 mmol, 3.00 eq) in acetonitrile (5.00 mL) was added phenyl chloroformate (0.14 mL, 1.09 mmol, 1.10 eq) in one portion at 25° C. The reaction was stirred at 25° C. for 12 h. The mixture was diluted with water (30.0 mL) and extracted with ethyl acetate (3×30.0 mL). The combined organic layers were washed with brine (2×10.0 mL), dried over sodium sulfate, filtered, and concentrated and the obtained residue was purified by standard methods to afford phenyl (3,3-dimethyl-2,3-dihydrobenzofuran-6-yl)carbamate.

Compound 284: General procedure A with variant i) was used for the preparation from compound VIII employing phenyl (6-(2-fluorophenyl)pyridin-3-yl)carbamate. ¹H NMR (400 MHz, DMSO-d₆) δ=11.02 (s, 1H), 10.23 (br s, 1H), 8.78 (d, J=2.3 Hz, 1H), 8.07-8.00 (m, 1H), 7.92 (dt, J=1.8, 7.9 Hz, 1H), 7.83 (s, 1H), 7.80-7.74 (m, 1H), 7.74-7.69 (m, 1H), 7.68-7.63 (m, 1H), 7.45 (ddt, J=1.8, 5.3, 7.6 Hz, 1H), 7.36-7.23 (m, 2H), 5.33 (s, 2H), 5.14 (dd, J=5.1, 13.4 Hz, 1H), 4.53-4.44 (m, 1H), 4.39-4.32 (m, 1H), 2.99-2.86 (m, 1H), 2.65-2.56 (m, 1H), 2.46-2.37 (m, 1H), 2.06-1.97 (m, 1H). MS (ESI) m/z 489.2 [M+H]⁺

Step 1: To a solution of 2-chloro-5-nitropyridine (2.00 g, 12.6 mmol, 1.00 eq) in dimethylformamide (20.0 mL) were added potassium carbonate (5.23 g, 37.8 mmol, 3.00 eq), (2-fluorophenyl)boronic acid (1.77 g, 12.6 mmol, 1.00 eq) and tetrakis(triphenylphosphine)palladium (728 mg, 630 μmol, 0.05 eq) under nitrogen. The reaction was stirred at 110° C. for 12 h. The mixture was diluted with water (200 mL) and extracted with ethyl acetate (2×100 mL). The combined organic layers were washed with brine (2×30.0 mL), dried over sodium sulfate, filtered, and concentrated under reduced pressure to give a residue. The residue was purified by column chromatography (petroleum ether/ethyl acetate=10/1 to 5/1) to afford 2-(2-fluorophenyl)-5-nitropyridine.

Step 2: To a solution of 2-(2-fluorophenyl)-5-nitropyridine (2.00 g, 9.17 mmol, 1.00 eq) in methanol (15.0 mL) and water (5.00 mL) were added iron powder (2.56 g, 45.8 mmol, 5.00 eq) and ammonium chloride (3.92 g, 73.3 mmol, 8.00 eq). The reaction was stirred at 80° C. for 1 h. The mixture was filtered, and the filtrate was concentrated under reduced pressure to give a residue. The residue was diluted with water (30.0 mL) and extracted with ethyl acetate (3×30.0 mL). The combined organic layers were washed with brine (2×10.0 mL), dried over sodium sulfate, filtered, and concentrated under reduced pressure to afford 6-(2-fluorophenyl)pyridin-3-amine.

Step 3: To a solution of 6-(2-fluorophenyl)pyridin-3-amine (1.70 g, 9.03 mmol, 1.00 eq) and pyridine (3.65 mL, 45.1 mmol, 5.00 eq) in acetonitrile (20.0 mL) was added phenyl chloroformate (1.60 mL, 12.8 mmol, 1.41 eq). The reaction was stirred at 25° C. for 1 h. The mixture was concentrated under reduced pressure to give a residue. The residue was triturated with water (100 mL) and the resulting solid was collected by filtration. The filter cake was washed with water (5 ml) and dried by standard methods to afford phenyl (6-(2-fluorophenyl)pyridin-3-yl)carbamate.

Compound 285: General procedure A with variant i) was used for the preparation from compound VIII employing phenyl (5-fluoro-6-phenylpyridin-3-yl)carbamate. ¹H NMR (400 MHz, DMSO-d₆) δ=11.08-10.94 (m, 1H), 10.57-10.27 (m, 1H), 8.59 (d, J=1.6 Hz, 1H), 8.00-7.92 (m, 1H), 7.91-7.85 (m, 2H), 7.83 (s, 1H), 7.75-7.70 (m, 1H), 7.68-7.64 (m, 1H), 7.53-7.47 (m, 2H), 7.46-7.40 (m, 1H), 5.35 (s, 2H), 5.14 (dd, J=5.2, 13.2 Hz, 1H), 4.57-4.44 (m, 1H), 4.43-4.30 (m, 1H), 2.98-2.83 (m, 1H), 2.61 (br d, J=17.2 Hz, 1H), 2.46-2.36 (m, 1H), 2.05-1.97 (m, 1H). MS (ESI) m/z 489.3[M+H]⁺

Step 1: To a solution of 2-chloro-3-fluoro-5-nitropyridine (2.00 g, 11.3 mmol, 1.00 eq), phenylboronic acid (2.76 g, 22.6 mmol, 2.00 eq) and potassium carbonate (4.70 g, 34.0 mmol, 3.00 eq) in dioxane (20.0 mL) was added tetrakis[triphenylphosphine]palladium(0) (1.31 g, 1.13 mmol, 0.10 eq) at 25° C. The reaction was stirred at 110° C. for 12 h under nitrogen. The mixture was poured into water (120 mL) and stirred for 10 min. The aqueous phase was extracted with ethyl acetate (3×60.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=50/1) to afford 3-fluoro-5-nitro-2-phenylpyridine.

Step 2: To a solution of 3-fluoro-5-nitro-2-phenylpyridine (300 mg, 1.37 mmol, 1.00 eq), in methanol (4.00 mL) and water (2.00 mL) were added ammonium chloride (367 mg, 6.87 mmol, 5.00 eq) and iron powder (230 mg, 4.12 mmol, 3.00 eq). The reaction was stirred at 80° C. for 1 h. The mixture was filtered, and the filtrate was concentrated under reduced pressure to give a residue. Water (80.0 mL) was added, and the solution was stirred for 10 min. The aqueous layer was extracted with ethyl acetate (3×30.0 mL). The combined organic layers were washed with brine (50.0 mL), dried over anhydrous sodium sulfate, filtered, and concentrated under reduced pressure to afford 5-fluoro-6-phenylpyridin-3-amine.

Step 3: To a solution of 5-fluoro-6-phenylpyridin-3-amine (250 mg, 1.33 mmol, 1.00 eq) and pyridine (0.54 mL, 6.64 mmol, 5.00 eq) in acetonitrile (5.00 mL) was added phenyl chloroformate (0.25 mL, 1.99 mmol, 1.50 eq). The reaction was stirred at 25° C. for 1 h. The mixture was concentrated and the obtained residue was purified by standard methods to afford phenyl (5-fluoro-6-phenylpyridin-3-yl)carbamate.

Compound 286: General procedure A with variant i) was used for the preparation from compound VIII employing phenyl (6-(o-tolyl)pyridin-3-yl)carbamate. ¹H NMR (400 MHz, DMSO-d₆) δ=11.02 (s, 1H), 10.69-10.33 (m, 1H), 8.95-8.73 (m, 1H), 8.35-8.00 (m, 1H), 7.86-7.63 (m, 4H), 7.47-7.29 (m, 4H), 5.36 (br s, 2H), 5.14 (dd, J=5.0, 13.4 Hz, 1H), 4.52-4.45 (m, 1H), 4.39-4.32 (m, 1H), 2.96-2.88 (m, 1H), 2.64-2.58 (m, 1H), 2.45-2.37 (m, 1H), 2.32 (s, 3H), 2.06-1.98 (m, 1H). MS (ESI) m/z 485.2 [M+H]⁺

Step 1: To a solution of 2-chloro-5-nitropyridine (3.00 g, 18.9 mmol, 1.00 eq) in dimethylformamide (30.0 mL) were added O-tolylboronic acid (2.57 g, 18.9 mmol, 1.00 eq), potassium carbonate (7.85 g, 56.7 mmol, 3.00 eq) and tetrakis(triphenylphosphine)palladium (1.09 g, 946 μmol, 0.05 eq) under nitrogen. The reaction was stirred at 110° C. for 12 h. The mixture was diluted with water (300 mL) and exacted with ethyl acetate (3×300 mL). The combined organic layers were washed with brine (2×100 mL), dried over sodium sulfate, filtered, and concentrated under reduced pressure to give a residue. The residue was purified by silica gel column chromatography (petroleum ether/ethyl acetate=10/1) to afford 5-nitro-2-(o-tolyl)pyridine.

Step 2: To a solution of 5-nitro-2-(o-tolyl)pyridine (3.00 g, 14.0 mmol, 1.00 eq) in methanol (30.0 mL) and water (10.0 mL) were added iron powder (3.91 g, 70.0 mmol, 5.00 eq) and ammonium chloride (5.99 g, 112 mmol, 8.00 eq). The reaction was stirred at 80° C. for 1 h. The mixture was filtered, and the filtrate was concentrated under reduced pressure to give a residue. The residue was diluted with water (50.0 mL) and exacted with ethyl acetate (3×30.0 mL). The combined organic layers were washed with brine (2×10.0 mL), dried over sodium sulfate, filtered, and concentrated under reduced pressure to afford 6-(o-tolyl)pyridin-3-amine.

Step 3: To a solution of 6-(o-tolyl)pyridin-3-amine (2.50 g, 13.5 mmol, 1.00 eq) in acetonitrile (25.0 mL) were added pyridine (5.48 mL, 67.8 mmol, 5.00 eq) and phenyl chloroformate (2.21 mL, 17.6 mmol, 1.30 eq). The reaction was stirred at 25° C. for 1 h. The mixture was concentrated under reduced pressure to give a residue. The residue was triturated with water (100 mL) and the resulting solid was collected by filtration. The filter cake was washed with water (5.00 ml) and dried under standard methods to afford phenyl (6-(o-tolyl)pyridin-3-yl)carbamate.

Compound 287: General procedure A with variant i) was used for the preparation from compound VIII employing phenyl (6-(2-methoxyphenyl)pyridin-3-yl)carbamate. ¹H NMR (400 MHz, DMSO-d₆) δ=11.02 (s, 1H), 10.75 (brs, 1H), 8.91 (brd, J=1.6 Hz, 1H), 8.32 (br d, J=8.7 Hz, 1H), 8.11 (d, J=8.9 Hz, 1H), 7.84 (s, 1H), 7.76-7.62 (m, 3H), 7.58-7.50 (m, 1H), 7.25 (d, J=8.3 Hz, 1H), 7.15 (t, J=7.5 Hz, 1H), 5.37 (s, 2H), 5.13 (dd, J=5.0, 13.3 Hz, 1H), 4.53-4.46 (m, 1H), 4.38-4.31 (m, 1H), 3.87 (s, 3H), 2.97-2.88 (m, 1H), 2.61 (br d, J=17.1 Hz, 1H), 2.45-2.35 (m, 1H), 2.07-1.97 (m, 1H). MS (ESI) m/z 501.2 [M+H]⁺

Step 1: To a solution of 2-chloro-5-nitropyridine (2.00 g, 12.6 mmol, 1.00 eq) in dimethylformamide (5 mL) were added potassium carbonate (5.23 g, 37.8 mmol, 3.00 eq), (2-fluorophenyl)boronic acid (1.92 g, 12.6 mmol, 1.00 eq) and tetrakis(triphenylphosphine)palladium (1.46 g, 1.26 mmol, 0.10 eq) under nitrogen. The reaction was stirred at 100° C. for 12 h. The mixture was diluted with water (300 mL) and exacted with ethyl acetate (3×300 mL). The combined organic layers were washed with brine (2×100 mL), dried over sodium sulfate, filtered, and concentrated under reduced pressure to give a residue. The residue was purified by silica gel column chromatography (petroleum ether/ethyl acetate=10/1) to afford 2-(2-methoxyphenyl)-5-nitropyridine.

Step 2: To a solution of 2-(2-methoxyphenyl)-5-nitropyridine (2.90 g, 12.6 mmol, 1.00 eq) in methanol (30.0 mL) and water (10.0 mL) were added iron powder (3.52 g, 62.9 mmol, 5.00 eq) and ammonium chloride (5.39 g, 100 mmol, 8.00 eq). The reaction was stirred at 80° C. for 1 h. The mixture was filtered, and the filtrate was concentrated under reduced pressure to give a residue. The residue was diluted with water (50.0 mL) and extracted with ethyl acetate (3×50.0 mL). The combined organic layers were washed with brine (2×10.0 mL), dried over sodium sulfate, filtered, and concentrated under reduced pressure to afford 6-(2-methoxyphenyl)pyridin-3-amine.

Step 3: To a solution of 6-(2-methoxyphenyl)pyridin-3-amine (2.50 g, 12.4 mmol, 1.00 eq) in acetonitrile (25.0 mL) were added pyridine (3.02 mL, 37.4 mmol, 3.00 eq) and phenyl chloroformate (2.03 mL, 16.2 mmol, 1.30 eq). The reaction was stirred at 25° C. for 1 h. The mixture was concentrated under reduced pressure to give a residue. The residue was triturated with water (100 mL) and the resulting solid was collected by filtration. The filter cake was washed with water (5.00 mL) and dried by standard methods to afford phenyl (6-(2-methoxyphenyl)pyridin-3-yl)carbamate.

Compound 288: General procedure A with variant i) was used for the preparation from compound VIII employing phenyl (5-methyl-6-phenylpyridin-3-yl)carbamate. ¹H NMR (400 MHz, DMSO-d₆) δ=11.02 (s, 1H), 10.76 (br s, 1H), 8.80-8.76 (m, 1H), 8.26 (s, 1H), 7.84 (s, 1H), 7.76-7.72 (m, 1H), 7.68-7.66 (m, 1H), 7.64-7.62 (m, 2H), 7.60 (br s, 3H), 5.38 (s, 2H), 5.22-5.04 (m, 1H), 4.50 (d, J=17.6 Hz, 1H), 4.36 (d, J=17.6 Hz, 1H), 2.98-2.86 (m, 1H), 2.68-2.58 (m, 1H), 2.46-2.34 (m, 4H), 2.10-1.98 (m, 1H). MS (ESI) m/z 485.4 [M+H]⁺

Step 1: To a solution of 2-chloro-3-methyl-5-nitropyridine (5.00 g, 29.0 mmol, 1.00 eq) and phenylboronic acid (4.24 g, 34.8 mmol, 1.20 eq) in dioxane (50.0 mL) were added tetrakis[triphenylphosphine]palladium(0) (6.70 g, 5.79 mmol, 0.20 eq) and potassium carbonate (6.01 g, 43.5 mmol, 1.50 eq) in one portion at 25° C. under nitrogen. The reaction was stirred at 100° C. for 12 h. The mixture was diluted with water (30.0 mL) and exacted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 3-methyl-5-nitro-2-phenyl-pyridine.

Step 2: To a solution of 3-methyl-5-nitro-2-phenylpyridine (1.00 g, 4.67 mmol, 1.00 eq) in methanol (6.00 mL) and water (3.00 mL) were added iron powder (1.30 g, 23.3 mmol, 5.00 eq) and ammonium chloride (1.25 g, 23.3 mmol, 5.00 eq) in one portion at 25° C. The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 5-methyl-6-phenylpyridin-3-amine.

Step 3: To a solution of 5-methyl-6-phenylpyridin-3-amine (843 mg, 4.58 mmol, 1.00 eq) and pyridine (1.11 mL, 13.7 mmol, 3.00 eq) in acetonitrile (2.00 mL) was added phenyl chloroformate (0.63 mL, 5.03 mmol, 1.10 eq) at 25° C. The reaction was stirred at 25° C. for 12 h. The mixture was concentrated and the obtained residue was purified by standard methods to afford phenyl (5-methyl-6-phenylpyridin-3-yl)carbamate.

Compound 289: General procedure A with variant i) was used for the preparation from compound VIII employing phenyl (6-(3-(dimethylamino)azetidin-1-yl)pyridin-3-yl)carbamate. ¹H NMR (400 MHz, DMSO-d₆) δ=10.99 (s, 1H), 9.52 (br s, 1H), 8.17 (s, 1H), 8.12 (br s, 1H), 7.77 (s, 1H), 7.69-7.56 (m, 3H), 6.37 (d, J=8.9 Hz, 1H), 5.24 (s, 2H), 5.12 (dd, J=5.1, 13.3 Hz, 1H), 4.51-4.43 (m, 1H), 4.38-4.30 (m, 1H), 3.92 (t, J=7.4 Hz, 2H), 3.63 (dd, J=5.6, 8.1 Hz, 2H), 3.15 (quin, J=6.1 Hz, 1H), 2.97-2.86 (m, 1H), 2.64-2.56 (m, 1H), 2.45-2.38 (m, 1H), 2.10 (s, 6H), 2.04-1.98 (m, 1H). MS (ESI) m/z 493.2 [M+H]⁺

Step 1: To a solution of 2-fluoro-5-nitropyridine (2.00 g, 14.1 mmol, 1.00 eq) and N,N-dimethylazetidin-3-amine dihydrochloride (3.65 g, 21.1 mmol, 1.50 eq, 2 HCl) in dimethylformamide (20.0 mL) was added potassium carbonate (9.73 g, 70.4 mmol, 5.00 eq) in one portion. The reaction was stirred at 60° C. for 12 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford N,N-dimethyl-1-(5-nitropyridin-2-yl)azetidin-3-amine.

Step 2: To a solution of N,N-dimethyl-1-(5-nitropyridin-2-yl)azetidin-3-amine (2.00 g, 9.00 mmol, 1.00 eq) in methanol (40.0 mL) was added palladium on carbon (200 mg, 10% weight on C) in one portion under hydrogen (15 Psi). The reaction was stirred at 25° C. for 2 h. The mixture was filtered, and the filtrate was concentrated under reduced pressure to afford 6-(3-(dimethylamino)azetidin-1-yl)pyridin-3-amine.

Step 3: To a solution of 6-(3-(dimethylamino)azetidin-1-yl)pyridin-3-amine (500 mg, 2.60 mmol, 1.00 eq) and pyridine (1.05 mL, 13.0 mmol, 5.00 eq) in acetonitrile (5.00 mL) was added phenyl chloroformate (0.39 mL, 3.12 mmol, 1.20 eq) dropwise at 0° C. The reaction was stirred at 20° C. for 12 h. The mixture was concentrated and the obtained residue was purified by standard methods to afford phenyl (6-(3-(dimethylamino)azetidin-1-yl)pyridin-3-yl)carbamate.

Compound 290: General procedure A with variant i) was used for the preparation from compound VIII employing phenyl (5-methoxy-6-phenylpyridin-3-yl)carbamate. ¹H NMR (400 MHz, DMSO-d₆) 5=11.02 (s, 1H), 10.80-10.36 (m, 1H), 8.60-8.38 (m, 1H), 8.12-7.92 (m, 1H), 7.88-7.78 (m, 3H), 7.76-7.64 (m, 2H), 7.50 (br d, J=7.2 Hz, 3H), 5.36 (s, 2H), 5.14 (dd, J=5.2, 13.2 Hz, 1H), 4.50 (d, J=17.6 Hz, 1H), 4.36 (d, J=17.6 Hz, 1H), 3.90 (s, 3H), 2.99-2.86 (m, 1H), 2.62 (br d, J=17.z Hz, 1H), 2.50-2.34 (m, 1H), 2.10-1.94 (m, 1H). MS (ESI) m/z 501.2 [M+H]⁺

Step 1: To a solution of 2-chloro-3-methoxy-5-nitropyridine (1.00 g, 5.30 mmol, 1.00 eq) and phenylboronic acid (776 mg, 6.36 mmol, 1.20 eq) in dioxane (50.0 mL) were added tetrakis[triphenylphosphine]palladium(0) (1.23 g, 1.06 mmol, 0.20 eq) and potassium carbonate (1.10 g, 7.95 mmol, 1.50 eq) in one portion at 25° C. under nitrogen. The reaction was stirred at 100° C. for 12 h. The mixture was diluted with water (30.0 mL) and exacted with ethyl acetate (3×30.0 mL). The combined organic layers were washed with brine (2×10.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 3-methoxy-5-nitro-2-phenylpyridine.

Step 2: To a solution of 3-methoxy-5-nitro-2-phenylpyridine (1.00 g, 4.34 mmol, 1.00 eq) in methanol (6.00 mL) and water (3.00 mL) were added iron powder (1.21 g, 21.7 mmol, 5.00 eq) and ammonium chloride (1.16 g, 21.7 mmol, 5.00 eq) in one portion at 25° C. The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over anhydrous sodium sulfate, filtered, and concentrated under reduced pressure to afford 5-methoxy-6-phenylpyridin-3-amine.

Step 3: To a solution of 5-methoxy-6-phenylpyridin-3-amine (700 mg, 3.50 mmol, 1.00 eq) and pyridine (0.85 mL, 10.5 mmol, 3.00 eq) in acetonitrile (2.00 mL) was added phenyl chloroformate (0.48 mL, 3.85 mmol, 1.10 eq) at 25° C. The reaction was stirred at 25° C. for 12 h. The mixture was concentrated and the obtained residue was purified by standard methods to afford phenyl (5-methoxy-6-phenylpyridin-3-yl)carbamate.

Compound 291: General procedure A with variant i) was used for the preparation from compound VIII employing phenyl (6-methyl-5-(trifluoromethoxy)pyridin-3-yl)carbamate. ¹H NMR (400 MHz, DMSO-d₆) δ=11.01 (br s, 1H), 10.30 (br s, 1H), 8.50 (d, J=2.0 Hz, 1H), 7.99 (br s, 1H), 7.80 (s, 1H), 7.72-7.67 (m, 1H), 7.66-7.62 (m, 1H), 5.30 (s, 2H), 5.16-5.09 (m, 1H), 4.52-4.43 (m, 1H), 4.38-4.30 (m, 1H), 2.97-2.85 (m, 1H), 2.65-2.63 (m, 1H), 2.65-2.58 (m, 1H), 2.40 (s, 3H), 2.40-2.33 (m, 1H), 2.05-1.97 (m, 1H). MS (ESI) m/z 493.2 [M+H]⁺

Step 1: To a solution of 2-methyl-5-nitropyridin-3-amine (10.0 g, 65.3 mmol, 1.00 eq) in sulfuric acid (2.50 M, 107 mL, 4.10 eq) was added sodium nitrite (5.41 g, 78.4 mmol, 1.20 eq) dissolved in water (20.0 mL) dropwise at 0° C. The reaction was stirred at 0° C. for 0.5 h. Then sulfuric acid (1 M, 53.6 mL, 0.820 eq) was added dropwise, and the reaction was stirred at 70° C. for 1 h. The mixture was diluted with water (200 mL) and extracted with ethyl acetate (3×50.0 mL). The combined organic layers were washed brine (40.0 mL), dried over sodium sulfate, filtered, and concentrated under reduced pressure to give a residue. The residue was purified by silica gel column chromatography (petroleum ether/ethyl acetate=10/1 to 2/1) to afford 2-methyl-5-nitropyridin-3-ol.

Step 2: To a solution of 2-methyl-5-nitropyridin-3-ol (800 mg, 5.19 mmol, 1.00 eq) in dimethylformamide (8.00 mL) was added sodium hydride (60% dispersion in mineral oil) (415 mg, 10.4 mmol, 2.00 eq) in portions at 0° C. The reaction was stirred at 0° C. for 0.5 h. Then dibromodifluoromethane (0.96 mL, 10.4 mmol, 2.00 eq) was added dropwise at 0° C., and the reaction was stirred at 25° C. for 2 h. The mixture was quenched with an ammonium chloride solution (100 mL) and extracted with ethyl acetate (3×50.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=10/1 to 5/1) to afford 3-(bromodifluoromethoxy)-2-methyl-5-nitropyridine.

Step 3: To a solution of 3-(bromodifluoromethoxy)-2-methyl-5-nitropyridine (180 mg, 636 μmol, 1.00 eq) in dichloromethane (3.00 mL) was added silver tetrafluoroborate (186 mg, 954 μmol, 1.50 eq) in portions. The reaction was stirred at 25° C. for 1 h. The mixture was filtered, and the filtrate was concentrated under reduced pressure to afford 2-methyl-5-nitro-3-(trifluoromethoxy)pyridine.

Step 4: A mixture of 2-methyl-5-nitro-3-(trifluoromethoxy)pyridine (100 mg, 450 μmol, 1.00 eq) and palladium on carbon (10% weight on C) (10.0 mg) in methanol (300 mL) was stirred at 25° C. for 2 h under hydrogen atmosphere (15 psi). The mixture was filtered over Celite, and the filtrate was concentrated under reduced pressure to afford 6-methyl-5-(trifluoromethoxy)pyridin-3-amine.

Step 5: A solution of phenyl chloroformate (16.0 μL, 124 μmol, 1.20 eq), 6-methyl-5-(trifluoromethoxy)pyridin-3-amine (20.0 mg, 104 μmol, 1.00 eq) and pyridine (25.0 μL, 312 μmol, 3.00 eq) in acetonitrile (10.0 mL) was stirred at 25° C. for 12 h. The mixture was concentrated and the obtained residue was purified by standard methods to afford phenyl (6-methyl-5-(trifluoromethoxy) pyridin-3-yl)carbamate.

Compound 292: General procedure A with variant i) was used for the preparation from compound VIII employing phenyl chroman-7-ylcarbamate. ¹H NMR (400 MHz, DMSO-d₆) δ=11.00 (s, 1H), 9.66 (br s, 1H), 7.79 (s, 1H), 7.72-7.61 (m, 2H), 7.00-6.84 (m, 3H), 5.25 (s, 2H), 5.13 (dd, J=5.2, 13.2 Hz, 1H), 4.53-4.42 (m, 1H), 4.40-4.29 (m, 1H), 4.16-4.02 (m, 2H), 2.99-2.85 (m, 1H), 2.68-2.58 (m, 3H), 2.41 (dq, J=4.4, 13.2 Hz, 1H), 2.07-1.97 (m, 1H), 1.95-1.83 (m, 2H). MS (ESI) m/z 450.2 [M+H]⁺

Step 1: To a mixture of chroman-6-amine (described in example 67) (1.20 g, 8.04 mmol, 1.00 eq) in dioxane (2.00 mL) was added acetic anhydride (1.51 mL, 16.1 mmol, 2.00 eq) and pyridine (0.65 mL, 8.04 mmol, 1.00 eq) at 0° C. The reaction was stirred at 25° C. for 16 h. The mixture was poured into water (20.0 mL) and extracted with ethyl acetate (3×20.0 mL). The combined organic layers were separated, washed with brine (10.0 mL), dried over anhydrous sodium sulfate, filtered, and concentrated under reduced pressure to afford N-(chroman-6-yl)acetamide.

Step 2: A solution of nitric acid (0.46 mL, 10.3 mmol, 1.40 eq) in acetic acid (2.00 mL) was added dropwise to a stirred solution of N-(chroman-6-yl)acetamide (1.40 g, 7.32 mmol, 1.00 eq) in acetic acid (10.0 mL) at 25° C. The reaction was stirred at 25° C. for 1 h. Then ice water (50.0 mL) was added, and the reaction was stirred at 25° C. for 0.5 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by reversed phase preparative HPLC to afford N-(7-nitrochroman-6-yl)acetamide carbamate.

Step 3: A solution of N-(7-nitrochroman-6-yl)acetamide (410 mg, 1.74 mmol, 1.00 eq) and concentrated hydrochloric acid (2.60 mL) in ethanol (10.0 mL) was stirred at 80° C. for 2 h. The reaction mixture was neutralized with ammonium hydroxide solution and diluted with water (20.0 mL), then extracted with ethyl acetate (3×20.0 mL). The combined organic layers were washed with brine (10.0 mL), dried over anhydrous sodium sulfate, filtered, and concentrated under reduced pressure to afford 7-nitrochroman-6-amine.

Step 4: To a mixture of 7-nitrochroman-6-amine (330 mg, 1.70 mmol, 1.00 eq) in tetrahydrofuran (10.0 mL) was added isoamyl nitrite (0.69 mL, 5.10 mmol, 3.00 eq) dropwise at 0° C. The reaction was stirred at 0° C. for 30 min, then at 70° C. for 3 h. The mixture was poured into water (20.0 mL) and extracted with ethyl acetate (3×20.0 mL). The combined organic layers were separated, washed with brine (10.0 mL), dried over anhydrous sodium sulfate, filtered, and concentrated under reduced pressure to give a residue. The residue was purified by silica gel column chromatography (petroleum ether/ethyl acetate=1/0 to 15/1) to afford 7-nitrochromane.

Step 5: To a solution of 7-nitrochromane (130 mg, 726 μmol, 1.00 eq) in ethyl acetate (6.00 mL) was added palladium on carbon (10% weight on C) (13.0 mg) under nitrogen atmosphere. The reaction was stirred at 25° C. for 2 h under hydrogen. The mixture was filtered, and the filtrate was concentrated under reduced pressure to afford chroman-7-amine.

Step 6: To a solution of chroman-7-amine (110 mg, 737 μmol, 1.00 eq) and pyridine (0.18 mL, 2.21 mmol, 3.00 eq) in acetonitrile (5.00 mL) was added phenyl chloroformate (0.11 mL, 885 μmol, 1.20 eq) at 25° C. The reaction was stirred at 25° C. for 12 h. The mixture was concentrated and the obtained residue was purified by standard methods to afford phenyl chroman-7-ylcarbamate.

Compound 293: General procedure A with variant i) was used for the preparation from compound VIII employing phenyl (3-cyclopropoxyphenyl)carbamate. ¹H NMR (400 MHz, DMSO-d₆) δ=11.00 (s, 1H), 9.81 (s, 1H), 7.80 (s, 1H), 7.72-7.62 (m, 2H), 7.28 (s, 1H), 7.21-7.14 (m, 1H), 7.05 (br d, J=8.3 Hz, 1H), 6.76-6.63 (m, 1H), 5.27 (s, 2H), 5.13 (dd, J=5.1, 13.3 Hz, 1H), 4.54-4.40 (m, 1H), 4.40-4.30 (m, 1H), 3.77 (td, J=3.0, 5.9 Hz, 1H), 3.01-2.83 (m, 1H), 2.61 (br dd, J=2.1, 15.7 Hz, 1H), 2.44-2.35 (m, 1H), 2.06-1.97 (m, 1H), 0.80-0.70 (m, 2H), 0.70-0.61 (m, 2H). MS (ESI) m/z 450.1 [M+H]⁺

Step 1: To a mixture of 3-nitrophenol (0.71 mL, 3.59 mmol, 1.00 eq) and bromocyclopropane (0.86 mL, 10.8 mmol, 3.00 eq) in 1-methyl-2-pyrrolidinone (5.00 mL) was added cesium carbonate (2.34 g, 7.19 mmol, 2.00 eq) in portions. The reaction was stirred at 180° C. for 2 h under microwave irradiation. The mixture was concentrated under reduced pressure to give a residue. The residue was purified by silica gel column chromatography (petroleum ether/ethyl acetate=20/1 to 5/1) to afford 1-cyclopropoxy-3-nitrobenzene.

Step 2: To a solution of 1-cyclopropoxy-3-nitrobenzene (240 mg, 1.34 mmol, 1.00 eq) in tetrahydrofuran (5.00 mL) was added Pd/C (10% weight on C) (50.0 mg) under nitrogen. The reaction was stirred at 20° C. for 1 h under hydrogen (15 psi). The mixture was filtered, and the filtrate was concentrated under reduced pressure to afford 3-cyclopropoxyaniline.

Step 3: To a solution of 3-cyclopropoxyaniline (280 mg, 1.88 mmol, 1.00 eq) and pyridine (0.76 mL, 9.38 mmol, 5.00 eq) in acetonitrile (3.00 mL) was added phenyl chloroformate (0.35 mL, 2.82 mmol, 1.50 eq). The reaction was stirred at 20° C. for 12 h. The mixture was concentrated and the obtained residue was purified by standard methods to afford phenyl (3-cyclopropoxyphenyl)carbamate.

Compound 294: General procedure A with variant i) was used for the preparation from compound VIII employing phenyl (5-(piperidin-1-yl)pyrazin-2-yl)carbamate. ¹H NMR (400 MHz, DMSO-d₆) δ=11.00 (s, 1H), 10.03 (s, 1H), 8.47 (s, 1H), 8.03 (d, J=1.2 Hz, 1H), 7.80 (s, 1H), 7.69-7.61 (m, 2H), 5.28 (s, 2H), 5.13 (dd, J=5.1, 13.2 Hz, 1H), 4.54-4.44 (m, 1H), 4.39-4.30 (m, 1H), 3.49 (br s, 4H), 2.97-2.89 (m, 1H), 2.63-2.58 (m, 1H), 2.43-2.35 (m, 1H), 2.05-1.97 (m, 1H), 1.61-1.51 (m, 6H). MS (ESI) m/z 479.2 [M+H]⁺

Step 1: To a solution of 5-chloropyrazin-2-amine (1.00 g, 7.72 mmol, 1.00 eq) and di-tert-butyldicarbonate (1.95 mL, 8.49 mmol, 1.10 eq) in tetrahydrofuran (10.0 mL) was added 4-dimethylaminopyridine (94.3 mg, 772 μmol, 0.10 eq). The reaction was stirred at 20° C. for 2 h. The mixture was concentrated under reduced pressure to give a residue. The residue was purified by silica gel column chromatography (petroleum ether/ethyl acetate=10/1 to 5/1) to afford tert-butyl (5-chloropyrazin-2-yl)carbamate.

Step 2: To a solution of tert-butyl (5-chloropyrazin-2-yl)carbamate (800 mg, 3.48 mmol, 1.00 eq) and piperidine (1.72 mL, 17.4 mmol, 5.00 eq) in dimethylformamide (3.00 mL) was added cesium carbonate (2.27 g, 6.97 mmol, 2.00 eq). The reaction was stirred at 180° C. for 2 h under microwave irradiation. The mixture was diluted with water (50.0 mL) and extracted with ethyl acetate (3×30.0 mL). The combined organic layers were washed with brine (20.0 mL), dried over anhydrous sodium sulfate, filtered, and concentrated under reduced pressure to give a residue. The residue was purified by reversed phase preparative HPLC to afford 5-(piperidin-1-yl) pyrazin-2-amine.

Step 3: To a solution of 5-(piperidin-1-yl)pyrazin-2-amine (50.0 mg, 281 μmol, 1.00 eq) and pyridine (0.07 mL, 842 μmol, 3.00 eq) in acetonitrile (1.00 mL) was added phenyl chloroformate (0.05 mL, 421 μmol, 1.50 eq). The reaction was stirred at 20° C. for 3 h. The mixture was concentrated and the obtained residue was purified by standard methods to afford phenyl (5-(piperidin-1-yl)pyrazin-2-yl) carbamate.

Compound 295: General procedure A with variant i) was used for the preparation from compound VIII employing phenyl (3-(difluoromethoxy)-5-(morpholinomethyl)phenyl)carbamate. ¹H NMR (400 MHz, DMSO-d₆) δ=11.00 (s, 1H), 10.01 (s, 1H), 8.19 (s, 1H), 7.80 (s, 1H), 7.71-7.62 (m, 2H), 7.29 (s, 2H), 7.17 (d, J=74 Hz, 1H), 6.76 (s, 1H), 5.29 (s, 2H), 5.13 (dd, J=5.1, 13.3 Hz, 1H), 4.52-4.44 (m, 1H), 4.39-4.31 (m, 1H), 3.60-3.55 (m, 4H), 3.42 (s, 2H), 2.97-2.87 (m, 1H), 2.64-2.58 (m, 1H), 2.45-2.40 (m, 1H), 2.35 (br s, 4H), 2.06-1.98 (m, 1H). MS (ESI) m/z 559.2 [M+H]⁺

Step 1: To a solution of 3-hydroxy-5-nitrobenzoic acid (1.80 g, 9.83 mmol, 1.00 eq) in dimethylformamide (10.0 mL) were added morpholine (0.86 mL, 9.83 mmol, 1.00 eq), 0-(7-azabenzotriazol-1-yl)-N,N,N′,N′-tetramethyluronium hexafluorophosphate (7.45 g, 19.6 mmol, 2.00 eq) and diisopropylethylamine (5.16 mL, 29.6 mmol, 3.00 eq). The reaction was stirred at 25° C. for 2 h. The mixture was extracted with ethyl acetate/water (200 ml/100 ml). The organic layer was collected, and the solvents were partly removed under reduced pressure to give a concentrated solution. The solution was purified by reversed phase preparative HPLC to afford (3-hydroxy-5-nitrophenyl)(morpholino)methanone.

Step 2: To a solution of (3-hydroxy-5-nitrophenyl)(morpholino)methanone (1.77 g, 7.02 mmol, 1.00 eq) in dimethylformamide (15.0 mL) were added (2-chloro-2,2-difluoro-acetyl)oxysodium (2.67 g, 17.5 mmol, 2.50 eq) and cesium carbonate (4.57 g, 14.0 mmol, 2.00 eq). The reaction was stirred at 100° C. for 2 h. The mixture was extracted with water/ethyl acetate (100 ml/100 ml). The organic layer was collected, and the solvents were partly removed under reduced pressure to give a concentrated solution. The solution was purified by reversed phase preparative HPLC to afford (3-(difluoromethoxy)-5-nitrophenyl)(morpholino)methanone.

Step 3: To a solution of (3-(difluoromethoxy)-5-nitrophenyl)(morpholino)methanone (1.39 g, 4.60 mmol, 1.00 eq) in tetrahydrofuran (5.00 mL) was added borane dimethyl sulfide complex (2 M in THF) (0.92 mL, 2.00 eq) at 25° C. The mixture was stirred at 25° C. for 0.5 h, then at 60° C. for 1.5 h. Methanol (5.00 mL) was added, and the mixture was extracted with water/ethyl acetate (50.0 ml/50.0 ml). The organic layer was collected, and the solvents were partly removed under reduced pressure to give a concentrated solution. The solution was purified by reversed phase preparative HPLC to afford 4-(3-(difluoromethoxy)-5-nitrobenzyl)morpholine.

Step 4: To a solution of 4-(3-(difluoromethoxy)-5-nitrobenzyl)morpholine (795 mg, 2.76 mmol, 1.00 eq) in methanol (15.0 mL) and water (5.00 mL) were added iron power (770 mg, 13.8 mmol, 5.00 eq) and ammonium chloride (1.18 g, 22.0 mmol, 8.00 eq). The reaction was stirred at 80° C. for 2 h. The mixture was filtered, and the filtrate was concentrated under reduced pressure to give a concentrated solution. The solution was extracted with ethyl acetate/saturated sodium bicarbonate (40.0 ml/10.0 ml). The organic layer was collected, dried over sodium sulfate, filtered, and concentrated under reduced pressure to afford 3-(difluoromethoxy)-5-(morpholinomethyl)aniline.

Step 5: To a solution of 3-(difluoromethoxy)-5-(morpholinomethyl)aniline (100 mg, 387 μmol, 1.00 eq) in acetonitrile (2.00 mL) were added pyridine (0.10 mL, 1.18 mmol, 3.04 eq) and phenyl chloroformate (0.07 mL, 599 μmol, 1.55 eq). The reaction was stirred at 25° C. for 2 h. The mixture was filtered, and the filtrate was purified by standard methods to afford phenyl (3-(difluoromethoxy)-5-(morpholinomethyl)phenyl)carbamate.

Compound 296: General procedure A with variant i) was used for the preparation from compound VIII employing phenyl(3-(difluoromethoxy)-5-(2-morpholinoethoxy)phenyl)carbamate. ¹H NMR (400 MHz, DMSO-d₆) δ=10.99 (s, 1H), 9.97 (s, 1H), 7.79 (s, 1H), 7.70-7.66 (m, 1H), 7.65-7.61 (m, 1H), 7.39-6.99 (m, 1H), 6.97-6.89 (m, 2H), 6.43 (t, J=2.1 Hz, 1H), 5.28 (s, 2H), 5.19-5.05 (m, 1H), 4.51-4.42 (m, 1H), 4.39-4.30 (m, 1H), 4.04 (t, J=5.7 Hz, 2H), 3.62-3.52 (m, 4H), 2.98-2.85 (m, 1H), 2.67 (t, J=5.6 Hz, 2H), 2.60 (br d, J=17.7 Hz, 1H), 2.48-2.40 (m, 4H), 2.40-2.31 (m, 1H), 2.06-1.96 (m, 1H). MS (ESI) m/z 589.4 [M+H]⁺

Step 1: A mixture of 3-bromo-5-nitrophenol (5.00 g, 22.9 mmol, 1.00 eq), 4-(2-chloroethyl)morpholine (4.12 g, 27.5 mmol, 1.20 eq) and caesium carbonate (22.4 g, 68.8 mmol, 3.00 eq) in dimethylformamide (50.0 mL) was stirred at 60° C. for 12 h. The mixture was diluted with ethyl acetate (250 mL) and water (150 mL). The organic layer was separated, and the aqueous layer was extracted with ethyl acetate (3×150 mL). The combined organic layers were dried over sodium sulfate, filtered, and concentrated under reduced pressure to give a residue. The residue was purified by column chromatography on silica gel (petroleum ether/ethyl acetate=3/1) to afford 4-(2-(3-bromo-5-nitrophenoxy)ethyl)morpholine.

Step 2: To a solution of 4-(2-(3-bromo-5-nitro-phenoxy)ethyl)morpholine (5.00 g, 15.1 mmol, 1.00 eq) and potassium hydroxide (2.54 g, 45.3 mmol, 3.00 eq) in dioxane (10.0 mL) and water (10.0 mL) were added tris(dibenzylideneacetone)dipalladium(0) (1.38 g, 1.51 mmol, 0.10 eq) and di-tert-butyl-(2-(2,4,6-tri(propan-2-yl)phenyl)phenyl)phosphane (641 mg, 1.51 mmol, 0.10 eq) under nitrogen. The reaction was stirred at 80° C. for 12 h. The mixture was diluted with ethyl acetate (150 mL) and water (150 mL). The organic layer was separated, and the aqueous layer was extracted with ethyl acetate (3×150 mL). The combined organic layers were dried over sodium sulfate, filtered, and concentrated under reduced pressure to give a residue. The residue was purified by reversed phase preparative HPLC to afford 3-(2-morpholinoethoxy)-5-nitrophenol.

Step 3: A mixture of 3-(2-morpholinoethoxy)-5-nitrophenol (300 mg, 1.12 mmol, 1.00 eq), potassium carbonate (309 mg, 2.24 mmol, 2.00 eq) and sodium 2-chloro-2,2-difluoroacetate (682 mg, 4.47 mmol, 4.00 eq) in dimethylformamide (2.00 mL) and water (0.50 mL) was stirred at 100° C. for 12 h. The mixture was diluted with ethyl acetate (100 mL) and water (100 mL). The organic layer was separated, and the aqueous layer was extracted with ethyl acetate (3×80.0 mL). The combined organic layers were dried over sodium sulfate, filtered, and concentrated under reduced pressure to afford 4-(2-(3-(difluoromethoxy)-5-nitrophenoxy)ethyl)morpholine.

Step 4: A mixture of 4-(2-(3-(difluoromethoxy)-5-nitrophenoxy)ethyl)morpholine (0.50 g, 1.57 mmol, 1.00 eq) and palladium on carbon (10% weight on C) (50.0 mg) in methanol (5.00 mL) was stirred at 25° C. under hydrogen for 3 h. The mixture was filtered over Celite, and the filtrate was concentrated under reduced pressure to afford 3-(difluoromethoxy)-5-(2-morpholinoethoxy)aniline.

Step 5: A mixture of 3-(difluoromethoxy)-5-(2-morpholinoethoxy)aniline (130 mg, 451 μmol, 1.00 eq), phenyl chloroformate (68.0 μL, 541 μmol, 1.20 eq) and pyridine (0.11 mL, 1.35 mmol, 3.00 eq) in acetonitrile (2.00 mL) was stirred at 25° C. for 12 h. The mixture was diluted with ethyl acetate (50.0 mL) and water (50.0 mL). The organic layer was separated, and the aqueous layer was extracted with ethyl acetate (3×100 mL). The combined organic layers were dried over sodium sulfate, filtered, and concentrated and the obtained residue was purified by standard methods to afford phenyl (3-(difluoromethoxy)-5-(2-morpholinoethoxy)phenyl)carbamate.

Compound 297: General procedure A with variant ii) was used for the preparation from compound VIII employing (2-methoxyphenyl)methanamine. ¹H NMR (400 MHz, DMSO-d₆) δ=10.98 (br s, 1H), 7.73 (s, 1H), 7.70 (br t, J=6.0 Hz, 1H), 7.61 (s, 2H), 7.25-7.13 (m, 2H), 6.98-6.86 (m, 2H), 5.16 (s, 2H), 5.14-5.08 (m, 1H), 4.49-4.42 (m, 1H), 4.36-4.29 (m, 1H), 4.18 (d, J=6.0 Hz, 2H), 3.78 (s, 3H), 2.97-2.85 (m, 1H), 2.60 (td, J=2.0, 15.4 Hz, 1H), 2.40 (br dd, J=4.4, 13.1 Hz, 1H), 2.05-1.96 (m, 1H). MS (ESI) m/z 438.1 [M+H]⁺

Compound 298: General procedure A with variant ii) was used for the from compound VIII employing phenylmethanamine. ¹H NMR (400 MHz, DMSO-d₆) δ=10.98 (br d, J=2.6 Hz, 1H), 7.90 (br t, J=6.2 Hz, 1H), 7.73 (s, 1H), 7.61 (s, 1H), 7.46-7.15 (m, 6H), 5.17 (s, 2H), 5.13 (dd, J=5.1, 13.4 Hz, 1H), 4.50-4.43 (m, 1H), 4.36-4.30 (m, 1H), 4.22 (d, J=6.1 Hz, 2H), 2.92 (ddd, J=5.4, 13.5, 17.4 Hz, 1H), 2.63-2.58 (m, 1H), 2.40 (br dd, J=4.4, 13.2 Hz, 1H), 2.05-1.98 (m, 1H). MS (ESI) m/z 408.1 [M+H]⁺

Compound 299: General procedure A with variant i) was used for the preparation from compound VIII employing phenyl (4-(1,1-difluoroethyl)phenyl)carbamate. ¹H NMR (400 MHz, DMSO-d₆) δ=11.00 (br s, 1H), 10.04 (s, 1H), 7.81 (s, 1H), 7.72-7.67 (m, 1H), 7.67-7.62 (m, 1H), 7.61-7.53 (m, 2H), 7.53-7.45 (m, 2H), 5.29 (s, 2H), 5.13 (dd, J=5.1, 13.3 Hz, 1H), 4.54-4.43 (m, 1H), 4.39-4.29 (m, 1H), 2.98-2.86 (m, 1H), 2.61 (td, J=2.1, 15.3 Hz, 1H), 2.44-2.35 (m, 1H), 2.06-1.99 (m, 1H), 1.99-1.88 (m, 3H). MS (ESI) m/z 438.1 [M+H]⁺

To a solution of 4-(1,1-difluoroethyl)aniline·HCl (50.0 mg, 258 μmol, 1.00 eq, HCl salt) in acetonitrile (5.00 mL) were added pyridine (0.10 mL, 1.29 mmol, 5.00 eq) and phenyl chloroformate (42.0 μL, 335 μmol, 1.30 eq). The reaction was stirred at 25° C. for 0.5 h. The mixture was concentrated under reduced pressure to give a residue. The residue was purified by preparative silica gel TLC to afford phenyl (4-(1,1-difluoroethyl)phenyl)carbamate.

Compound 300: General procedure A with variant i) was used for the preparation from compound VIII employing phenyl (3-(2-oxa-5-azabicyclo[2.2.1]heptan-5-ylmethyl)-5-(difluoromethoxy)phenyl) carbamate. ¹H NMR (400 MHz, DMSO-d₆) δ=11.00 (s, 1H), 9.99 (s, 1H), 8.17 (s, 1H), 7.80 (s, 1H), 7.71-7.62 (m, 2H), 7.32 (s, 1H), 7.26 (s, 1H), 7.13 (d, J=74 Hz, 1H), 6.78 (s, 1H), 5.29 (s, 2H), 5.13 (dd, J=5.1, 13.2 Hz, 1H), 4.46 (s, 1H), 4.38-4.32 (m, 2H), 3.90 (d, J=7.5 Hz, 1H), 3.68 (d, J=4.5 Hz, 2H), 3.53 (dd, J=1.6, 7.4 Hz, 1H), 3.45 (s, 1H), 2.97-2.88 (m, 1H), 2.71 (s, 1H), 2.63 (br s, 1H), 2.41 (br d, J=9.4 Hz, 2H), 2.05-1.99 (m, 1H), 1.80 (br d, J=8.2 Hz, 1H), 1.60 (br d, J=9.5 Hz, 1H). MS (ESI) m/z 571.2 [M+H]⁺

Step 1: To a solution of 3-hydroxy-5-nitrobenzoic acid (2.80 g, 15.3 mmol, 1.00 eq) in dimethylformamide (5.00 mL) were added 2-oxa-5-azabicyclo[2.2.1]heptane·HCl (3.11 g, 22.9 mmol, 1.50 eq, HCl), O-(7-azabenzotriazol-1-yl)-N,N,N′,N′-tetramethyluronium hexafluorophosphate (11.6 g, 30.6 mmol, 2.00 eq) and diisopropylethylamine (8.00 mL, 45.8 mmol, 3.00 eq). The reaction was stirred at 25° C. for 2 h. The mixture was extracted with water/ethyl acetate (100 ml/200 ml). The organic layer was separated, and most of the solvent was removed under reduced pressure to give a concentrated solution. The solution was purified by reversed phase preparative HPLC to afford 2-oxa-5-azabicyclo[2.2.1]heptan-5-yl(3-hydroxyl-5-nitrophenyl)methanone.

Step 2: To a solution of 2-oxa-5-azabicyclo[2.2.1]heptan-5-yl(3-hydroxy-5-nitrophenyl)methanone (3.79 g, 14.3 mmol, 1.00 eq) in dimethylformamide (150 mL) were added (2-chloro-2,2-difluoro-acetyl)oxysodium (5.47 g, 35.8 mmol, 2.50 eq) and cesium carbonate (9.35 g, 28.7 mmol, 2.00 eq). The reaction was stirred at 100° C. for 2 h. The mixture was extracted with water/ethyl acetate (100 ml/100 ml). The organic layer was separated, and most of the solvent was removed under reduced pressure to give a concentrated solution. The solution was purified by reversed phase preparative HPLC to afford 2-oxa-5-azabicyclo[2.2.1]heptan-5-yl(3-(difluoromethoxy)-5-nitrophenyl)methanone.

Step 3: To a solution of 2-oxa-5-azabicyclo[2.2.1]heptan-5-yl(3-(difluoromethoxy)-5-nitrophenyl)methanone (1.82 g, 5.79 mmol, 1.00 eq) in tetrahydrofuran (5.00 mL) was added borane dimethyl sulfide complex (10 M) (1.16 mL, 2.00 eq) at 25° C. under nitrogen. The reaction was stirred at 25° C. for 0.5 h, then at 60° C. for 1.5 h. Methanol (5.00 ml) was added, and the mixture was extracted with water/ethyl acetate (50.0 ml/50.0 ml). The organic layer was separated, and most of the solvent was removed under reduced pressure to give a concentrated solution. The solution was purified by reversed phase preparative HPLC to afford 5-(3-(difluoromethoxy)-5-nitrobenzyl)-2-oxa-5-azabicyclo[2.2.1]heptane.

Step 4: To a solution of 5-(3-(difluoromethoxy)-5-nitrobenzyl)-2-oxa-5-azabicyclo[2.2.1]heptane (902 mg, 3.00 mmol, 1.00 eq) in methanol (15.0 mL) and water (5.00 mL) were added iron power (839 mg, 15.0 mmol, 5.00 eq) and ammonium chloride (1.29 g, 24.0 mmol, 8.00 eq). The reaction was stirred at 80° C. for 2 h. The mixture was filtered, and the filtrate was concentrated under reduced pressure to give a concentrated solution. The solution was extracted with ethyl acetate/saturated sodium bicarbonate (40.0 mL/10.0 mL). The organic layer was collected, dried over sodium sulfate, filtered, and concentrated under reduced pressure to afford 3-(2-oxa-5-azabicyclo[2.2.1]heptan-5-ylmethyl)-5-(difluoromethoxy)aniline.

Step 5: To a solution of 3-(2-oxa-5-azabicyclo[2.2.1]heptan-5-ylmethyl)-5-(difluoromethoxy)aniline (200 mg, 740 μmol, 1.00 eq) in acetonitrile (2.00 mL) were added pyridine (0.18 mL, 2.23 mmol, 3.00 eq) and phenyl chloroformate (0.14 mL, 1.12 mmol, 1.50 eq). The reaction was stirred at 25° C. for 2 h. The mixture was filtered, and the filtrate was purified by standard methods to afford phenyl (3-(2-oxa-5-azabicyclo[2.2.1]heptan-5-ylmethyl)-5-(difluoromethoxy)phenyl)carbamate.

Compound 301: General procedure A with variant i) was used for the preparation from compound VIII employing phenyl (4-(2,2-difluorocyclopropyl)phenyl)carbamate. ¹H NMR (400 MHz, DMSO-d₆) δ=10.99 (s, 1H), 9.82 (br s, 1H), 7.79 (s, 1H), 7.71-7.58 (m, 2H), 7.44 (br d, J=8.6 Hz, 2H), 7.18 (d, J=8.6 Hz, 2H), 5.27 (s, 2H), 5.13 (dd, J=5.0, 13.2 Hz, 1H), 4.52-4.42 (m, 1H), 4.39-4.30 (m, 1H), 2.96-2.86 (m, 2H), 2.62 (br s, 1H), 2.46-2.36 (m, 1H), 2.05-1.97 (m, 1H), 1.96-1.81 (m, 2H). MS (ESI) m/z 470.1 [M+H]⁺

Step 1: To a solution of 1-nitro-4-vinylbenzene (500 mg, 3.35 mmol, 1.00 eq) and sodium iodide (251 mg, 1.68 mmol, 0.50 eq) in 1,2-dimethoxyethane (5.00 mL) was added (trifluoromethyl)trimethylsilane (1.19 g, 8.38 mmol, 2.50 eq) dropwise under nitrogen. The reaction was stirred at 150° C. for 2 h under microwave irradiation. The mixture was concentrated under reduced pressure to give a residue. The residue was purified by silica gel column chromatography (petroleum ether/ethyl acetate=10/1) to afford 1-(2,2-difluorocyclopropyl)-4-nitrobenzene.

Step 2: To a solution of 1-(2,2-difluorocyclopropyl)-4-nitrobenzene (380 mg, 1.91 mmol, 1.00 eq) in ethanol (8.00 mL) and water (2.00 mL) were added iron power (533 mg, 9.54 mmol, 5.00 eq) and ammonium chloride (510 mg, 9.54 mmol, 5.00 eq) in one portion. The reaction was stirred at 80° C. for 1 h. The mixture was diluted with water (80.0 mL) and extracted with ethyl acetate (3×60.0 mL). The combined organic layers were washed with brine (30.0 mL), dried over sodium sulfate, filtered, and concentrated under reduced pressure to afford 4-(2,2-difluorocyclopropyl)aniline.

Step 3: To a solution of 4-(2,2-difluorocyclopropyl)aniline (300 mg, 1.77 mmol, 1.00 eq) and pyridine (0.72 mL, 8.87 mmol, 5.00 eq) in acetonitrile (5.00 mL) was added phenyl chloroformate (0.27 mL, 2.13 mmol, 1.20 eq) dropwise at 0° C. The reaction was stirred at 25° C. for 12 h. The mixture was concentrated and the obtained residue was purified by standard methods to afford phenyl (4-(2,2-difluorocyclopropyl) phenyl)carbamate.

Compound 302: General procedure A with variant ii) was used for the preparation from compound VIII employing phenylmethanamine. ¹H NMR (400 MHz, DMSO-d₆) δ=10.98 (br s, 1H), 7.89 (br d, J=8.2 Hz, 1H), 7.72 (s, 1H), 7.59 (s, 2H), 7.30 (d, J=4.4 Hz, 4H), 7.23-7.18 (m, 1H), 5.15-5.07 (m, 3H), 4.67 (quin, J=7.4 Hz, 1H), 4.48-4.41 (m, 1H), 4.35-4.28 (m, 1H), 2.96-2.86 (m, 1H), 2.63-2.57 (m, 1H), 2.39 (br dd, J=4.4, 13.2 Hz, 1H), 2.04-1.97 (m, 1H), 1.34 (d, J=7.0 Hz, 3H). MS (ESI) m/z 422.2 [M+H]⁺

Compound 303: General procedure A with variant i) was used for the preparation from compound VIII employing phenyl (3-methyl-2,3-dihydrobenzofuran-6-yl)carbamate. ¹H NMR (400 MHz, DMSO-d₆) δ=11.10-10.94 (m, 1H), 9.81-9.65 (m, 1H), 8.45 (s, 1H), 7.79 (s, 1H), 7.70-7.61 (m, 2H), 7.15-7.03 (m, 1H), 6.99-6.83 (m, 1H), 5.25 (s, 2H), 5.13 (dd, J=5.1, 13.2 Hz, 1H), 4.64 (t, J=8.8 Hz, 1H), 4.53-4.44 (m, 1H), 4.40-4.29 (m, 1H), 4.00 (dd, J=7.5, 8.5 Hz, 1H), 3.50-3.46 (m, 1H), 2.95-2.87 (m, 1H), 2.62 (br d, J=2.5 Hz, 1H), 2.41-2.31 (m, 1H), 2.04-1.98 (m, 1H), 1.22 (d, J=6.9 Hz, 3H). MS (ESI) m/z 450.1 [M+H]⁺

Step 1: To a solution of 2-bromo-5-nitro-phenol (1.00 g, 4.59 mmol, 1.00 eq) and potassium carbonate (1.27 g, 9.17 mmol, 2.00 eq) in acetone (10.0 mL) was added 3-bromoprop-1-ene (665 mg, 5.50 mmol, 1.20 eq) at 25° C. The reaction was stirred at 25° C. for 12 h. The mixture was concentrated under reduced pressure to give a residue. Water (80.0 m L) was added, and the mixture was stirred for 10 min. The aqueous layer was extracted with ethyl acetate (3×40.0 mL). The combined organic layers were washed with brine (100 mL), dried over anhydrous sodium sulfate, filtered, and concentrated under reduced pressure to give a residue. The residue was purified by silica gel column chromatography (petroleum ether/ethyl acetate=50/1) to afford 2-(allyloxy)-1-bromo-4-nitrobenzene.

Step 2: To a solution of 2-(allyloxy)-1-bromo-4-nitrobenzene (800 mg, 3.10 mmol, 1.00 eq), sodium acetate (635 mg, 7.75 mmol, 2.50 eq), tetraethylammonium iodide (877 mg, 3.41 mmol, 1.10 eq) and sodium formate (210 mg, 3.10 mmol, 1.00 eq) in dimethylformamide (2.00 mL) was added palladium acetate (139 mg, 620 μmol, 0.20 eq) at 25° C. The reaction was stirred at 100° C. for 12 h. The mixture was poured into water (80.0 mL) and stirred for 10 min. The aqueous layer was extracted with ethyl acetate (3×30.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=20/1) to afford 3-methyl-6-nitrobenzofuran.

Step 3: To a solution of 3-methyl-6-nitro-benzofuran (150 mg, 846 μmol, 1.00 eq) in methanol (2.00 mL) was added palladium on carbon (10% weight on C) (30.0 mg). The reaction was stirred at 25° C. for 12 h under hydrogen. The mixture was filtered, and the filtrate was concentrated under reduced pressure to afford 3-methyl-2,3-dihydrobenzofuran-6-amine.

Step 4: To a solution of 3-methyl-2,3-dihydrobenzofuran-6-amine (120 mg, 804 μmol, 1.00 eq) and pyridine (0.32 mL, 4.02 mmol, 5.00 eq) in acetonitrile (3.00 mL) was added phenyl chloroformate (0.15 mL, 1.21 mmol, 1.50 eq). The reaction was stirred at 25° C. for 12 h. The mixture was concentrated and the obtained residue was purified by standard methods to afford phenyl (3-methyl-2,3-dihydrobenzofuran-6-yl)carbamate.

Compound 304: General procedure A with variant i) was used for the preparation from compound VIII employing phenyl N-(4-cyclobutylphenyl)carbamate. ¹H NMR (400 MHz, DMSO-d₆) δ=11.00 (s, 1H), 9.72 (br s, 1H), 7.79 (s, 1H), 7.72-7.59 (m, 2H), 7.39 (br d, J=8.4 Hz, 2H), 7.15 (d, J=8.4 Hz, 2H), 5.26 (s, 2H), 5.13 (dd, J=5.2, 13.2 Hz, 1H), 4.55-4.43 (m, 1H), 4.40-4.26 (m, 1H), 3.47-3.44 (m, 1H), 2.97-2.83 (m, 1H), 2.63 (br s, 1H), 2.45-2.35 (m, 1H), 2.30-2.21 (m, 2H), 2.09-1.98 (m, 3H), 1.97-1.87 (m, 1H), 1.84-1.73 (m, 1H). MS (ESI) m/z 448.1 [M+H]⁺

Step 1: To a solution of 4-bromoaniline (10.0 g, 58.1 mmol, 1.00 eq) and triethylamine (24.0 mL, 172 mmol, 2.97 eq) in dichloromethane (70.0 mL) was added trifluoroacetic anhydride (12.1 mL, 87.2 mmol, 1.50 eq) dropwise at 0° C. The reaction was stirred at 25° C. for 12 h. The mixture was purified by silica gel column chromatography (petroleum ether/ethyl acetate=10/1) to afford N-(4-bromophenyl)-2,2,2-trifluoro-acetamide.

Step 2: To a solution of N-(4-bromophenyl)-2,2,2-trifluoro-acetamide (2.00 g, 7.46 mmol, 1.00 eq) in tetrahydrofuran (15.0 mL) was added n-Butyllithium (2.50 M, 6.27 mL, 2.10 eq) dropwise at −78° C. The reaction was stirred at −78° C. for 0.5 h. Then cyclobutanone (0.67 mL, 8.95 mmol, 1.20 eq) was added, and the reaction was stirred at −78° C. for 2.5 h. The reaction was quenched by addition of saturated aqueous ammonium chloride, and the mixture was extracted with ethyl acetate (2×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 2,2,2-trifluoro-N-(4-(1-hydroxycyclobutyl)phenyl)acetamide.

Step 3: To a solution of 2,2,2-trifluoro-N-(4-(1-hydroxycyclobutyl)phenyl)acetamide (1.60 g, 6.17 mmol, 1.00 eq) in methanol (10.0 mL) was added sodium hydroxide solution (1.00 M, 6.17 mL, 1.00 eq). The reaction was stirred at 25° C. for 12 h. The mixture was concentrated under reduced pressure to give a residue. Water (10.0 mL) was added, and the mixture was extracted with ethyl acetate (2×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=5/1 to 2/1) to afford 1-(4-aminophenyl)cyclobutanol.

Step 4: To a solution of 1-(4-aminophenyl)cyclobutanol (0.70 g, 4.29 mmol, 1.00 eq) in tetrahydrofuran (10.0 mL) were added sodium borohydride (923 mg, 24.4 mmol, 5.69 eq) and aluminium trichloride (1.72 g, 12.9 mmol, 3.00 eq) in portions. The reaction was stirred at 70° C. for 3 h. The mixture was poured into water (100 mL) in portions. The aqueous layer was extracted with ethyl acetate (2×20.0 mL). The combined organic layers were washed with brine (30.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 4-cyclobutylaniline.

Step 5: To a solution of 4-cyclobutylaniline (200 mg, 1.36 mmol, 1.00 eq) and pyridine (0.55 mL, 6.79 mmol, 5.00 eq) in acetonitrile (3.00 mL) was added phenyl chloroformate (0.20 mL, 1.63 mmol, 1.20 eq). The reaction was stirred at 25° C. for 1 h. The mixture was concentrated and the obtained residue was purified by standard methods to afford phenyl N-(4-cyclobutylphenyl)carbamate.

Compound 305: General procedure A with variant i) was used for the preparation from compound VIII employing phenyl (6-cyclobutylpyridin-3-yl)carbamate. ¹H NMR (400 MHz, DMSO-d₆) δ=10.99 (s, 1H), 10.81 (s, 1H), 8.80 (d, J=2.1 Hz, 1H), 8.37 (dd, J=2.3, 8.8 Hz, 1H), 7.95 (d, J=8.8 Hz, 1H), 7.81 (s, 1H), 7.73-7.68 (m, 1H), 7.67-7.61 (m, 1H), 5.35 (s, 2H), 5.11 (dd, J=5.1, 13.3 Hz, 1H), 4.52-4.45 (m, 1H), 4.38-4.31 (m, 1H), 3.92 (br d, J=8.8 Hz, 1H), 2.96-2.87 (m, 1H), 2.60 (br d, J=17.5 Hz, 1H), 2.43-2.32 (m, 5H), 2.10-1.98 (m, 2H), 1.88 (dtd, J=3.2, 7.9, 11.1 Hz, 1H). MS (ESI) m/z 449.2 [M+H]⁺

Step 1: To a solution of zinc bromide (2.49 g, 11.0 mmol, 3.50 eq) in tetrahydrofuran (15.0 mL) was added cyclobutylmagnesium bromide (0.5 M, 17.6 mL, 2.80 eq) dropwise at −78° C. under nitrogen. The reaction was stirred at −78° C. for 0.5 h, then at 0° C. for 0.5 h. 2-chloro-5-nitropyridine (500 mg, 3.15 mmol, 1.00 eq) and tetrakis(triphenylphosphine)palladium(0) (364 mg, 315 μmol, 0.10 eq) were added successively. The reaction was stirred at 0° C. for 10 min. Then the reaction was warmed to 60° C. and stirred at 60° C. for 2 h. The mixture was diluted with saturated ammonium chloride (100 mL) and extracted with ethyl acetate (3×80.0 mL). The combined organic layers were washed with brine (60.0 mL), dried over sodium sulfate, filtered, and concentrated under reduced pressure to give a residue. The residue was purified by silica gel column chromatography (petroleum ether/ethyl acetate=5/1) to afford 2-cyclobutyl-5-nitro-pyridine.

Step 2: To a solution of 2-cyclobutyl-5-nitropyridine (320 mg, 1.80 mmol, 1.00 eq) in methanol (10.0 mL) was added palladium on carbon (10% weight on C) (50.0 mg) in one portion. The reaction was stirred at 25° C. for 2 h under hydrogen (15 Psi). The mixture was filtered, and the filtrate was concentrated under reduced pressure to afford 6-cyclobutylpyridin-3-amine.

Step 3: To a solution of 6-cyclobutylpyridin-3-amine (230 mg, 1.55 mmol, 1.00 eq) and pyridine (0.63 mL, 7.76 mmol, 5.00 eq) in acetonitrile (5.00 mL) was added phenyl chloroformate (0.23 mL, 1.86 mmol, 1.20 eq) dropwise at 0° C. The reaction was stirred at 25° C. for 12 h. The mixture was concentrated under reduced pressure to give a residue. Dimethylformamide (2.00 mL) was added, and the solution was filtered. The filtrate was concentrated by standard methods to afford phenyl (6-cyclobutylpyridin-3-yl)carbamate.

Compound 306: General procedure A with variant i) was used for the preparation from compound VIII employing phenyl (3-(2-morpholinoethoxy)-5-(trifluoromethoxy)phenyl)carbamate. ¹H NMR (400 MHz, DMSO-d₆) δ=11.09-10.90 (m, 1H), 10.08 (s, 1H), 8.23 (br s, 1H), 7.79 (s, 1H), 7.67 (br d, J=1.1 Hz, 1H), 7.66-7.62 (m, 1H), 7.14 (s, 1H), 7.07 (s, 1H), 6.59 (s, 1H), 5.28 (s, 2H), 5.17-5.04 (m, 1H), 4.51-4.43 (m, 1H), 4.38-4.30 (m, 1H), 4.06 (t, J=5.7 Hz, 2H), 3.60-3.53 (m, 4H), 2.97-2.86 (m, 1H), 2.67 (t, J=5.6 Hz, 2H), 2.64-2.57 (m, 1H), 2.48-2.42 (m, 4H), 2.40-2.31 (m, 1H), 2.05-1.97 (m, 1H). MS (ESI) m/z 607.4 [M+H]⁺

Step 1: To a solution of 3-(2-morpholinoethoxy)-5-nitrophenol (110 mg, 410 μmol, 1.00 eq) in dimethylformamide (3.00 mL) was added sodium hydride (60% dispersion in mineral oil) (32.8 mg, 820 μmol, 2.00 eq) in portions at 0° C. The reaction was stirred at 0° C. for 0.5 h, then dibromodifluoromethane (75.8 μL, 820 μmol, 2.00 eq) was added dropwise. The reaction was stirred at 25° C. for 1 h. Saturated aqueous ammonium chloride solution (20.0 mL) was added, and the mixture was extracted with ethyl acetate (2×20.0 mL). The combined organic layers were washed with brine (10.0 mL), dried over sodium sulfate, filtered, and concentrated under reduced pressure to give a residue. The residue was purified by preparative silica gel TLC (petroleum ether/ethyl acetate=1/1) to afford 4-(2-(3-(bromodifluoromethoxy)-5-nitrophenoxy)ethyl)morpholine.

Step 2: To a solution of 4-(2-(3-(bromodifluoromethoxy)-5-nitrophenoxy)ethyl)morpholine (90.0 mg, 227 μmol, 1.00 eq) in dichloromethane (3.00 mL) was added silver tetrafluoroborate (88.2 mg, 453 μmol, 2.00 eq) in portions. The reaction was stirred at 25° C. for 1 h. The mixture was filtered, and the filtrate was concentrated under reduced pressure to afford 4-(2-(3-nitro-5-(trifluoromethoxy)phenoxy)ethyl)morpholine.

Step 3: A mixture of 4-(2-(3-nitro-5-(trifluoromethoxy)phenoxy)ethyl)morpholine (300 mg, 892 μmol, 1.00 eq) and palladium on carbon (10% weight on C) (100 mg) in methanol (10.0 mL) was stirred at 25° C. under hydrogen for 3 h. The mixture was filtered over Celite, and the filtrate was concentrated under reduced pressure to afford 3-(2-morpholinoethoxy)-5-(trifluoromethoxy)aniline.

Step 4: A solution of 3-(2-morpholinoethoxy)-5-(trifluoromethoxy)aniline (110 mg, 359 μmol, 1.00 eq), phenyl chloroformate (67.5 μL, 539 μmol, 1.50 eq) and pyridine (87.0 μL, 1.08 mmol, 3.00 eq) in acetonitrile (10.0 mL) was stirred at 25° C. for 12 h. The mixture was diluted with ethyl acetate (30.0 mL) and water (30.0 mL). The organic layer was separated, and the aqueous phase was extracted with ethyl acetate (3×30.0 mL). The combined organic layers were dried over sodium sulfate, filtered, and concentrated and the obtained residue was purified by standard methods to afford phenyl (3-(2-morpholinoethoxy)-5-(trifluoromethoxy) phenyl)carbamate.

Compound 307: General procedure A with variant i) was used for the preparation from compound VIII employing phenyl (6-(azetidin-1-yl)pyridin-3-yl)carbamate. ¹H NMR (400 MHz, DMSO-d₆) δ=10.99 (s, 1H), 10.12-9.91 (m, 1H), 8.09 (br s, 1H), 7.84 (br d, J=7.8 Hz, 1H), 7.78 (s, 1H), 7.71-7.60 (m, 2H), 6.80 (br d, J=7.9 Hz, 1H), 5.28 (s, 2H), 5.12 (dd, J=5.1, 13.4 Hz, 1H), 4.51-4.41 (m, 1H), 4.38-4.29 (m, 1H), 4.27-4.06 (m, 4H), 2.96-2.87 (m, 1H), 2.63 (br s, 1H), 2.47-2.33 (m, 3H), 2.05-1.98 (m, 1H). MS (ESI) m/z 450.1 [M+H]⁺

Step 1: To a solution of azetidine·HCl (5.14 mL, 46.5 mmol, 1.50 eq, HCl) and 2-fluoro-5-nitropyridine (4.40 g, 30.9 mmol, 1.00 eq) in dimethylformamide (40.0 mL) was added potassium carbonate (12.8 g, 92.9 mmol, 3.00 eq) in one portion. The reaction was stirred at 60° C. for 12 h. The mixture was poured into water (60.0 mL) and filtered. The filtrate was concentrated under reduced pressure to afford 2-(azetidin-1-yl)-5-nitropyridine.

Step 2: To a solution of 2-(azetidin-1-yl)-5-nitropyridine (2.00 g, 11.2 mmol, 1.00 eq) in methanol (40.0 mL) was added palladium on carbon (10% weight on C) (300 mg). The reaction was stirred at 25° C. for 2 h under hydrogen (15 Psi). The mixture was filtered, and the filtrate was concentrated under reduced pressure to afford 6-(azetidin-1-yl)pyridin-3-amine.

Step 3: To a solution of 6-(azetidin-1-yl)pyridin-3-amine (700 mg, 4.69 mmol, 1.00 eq) and pyridine (1.89 mL, 23.5 mmol, 5.00 eq) in acetonitrile (10.0 mL) was added phenyl chloroformate (0.71 mL, 5.63 mmol, 1.20 eq) dropwise at 0° C. The reaction was stirred at 25° C. for 12 h. The mixture was concentrated and the obtained residue was purified by standard methods to afford phenyl (6-(azetidin-1-yl)pyridin-3-yl)carbamate.

### Compound 308:

Step 1: To a solution of 4-bromo-3,5-dimethylaniline (1.00 g, 5.00 mmol, 1.00 eq) in dimethyl formamide (10.0 mL) was added zinc cyanide (704 mg, 6.00 mmol, 381 uL, 1.20 eq) and 1,1-bis(diphenylphosphino)ferrocene (416 mg, 750 umol, 0.150 eq), tris(dibenzylideneacetone)dipalladium(0) (458 mg, 500 umol, 0.100 eq) under nitrogen. The mixture was stirred at 150° C. for 2 h under nitrogen in microwave. The mixture was filtered. The filtrate was diluted with water and ethyl acetate (50.0 ml/50.0 ml). The organic layer was collected, concentrated and purified by column chromatography on silica gel. The desired fraction was collected and concentrated to give 4-amino-2,6-dimethylbenzonitrile. ¹H NMR (400 MHz, DMSO-d₆) δ=6.31 (s, 2H), 5.93 (s, 2H), 2.26 (s, 6H).

Step 2: To a solution of 4-amino-2,6-dimethylbenzonitrile (357 mg, 2.44 mmol, 1.00 eq) in Acetonitrile (5.00 mL) was added pyridine (578 mg, 7.31 mmol, 590 uL, 2.99 eq) and phenyl carbonochloridate (459 mg, 2.93 mmol, 367 uL, 1.20 eq). The mixture was stirred at 10° C. for 1 h. The mixture was filtered to give filtrate. The filtrate was purified by reversed-phase HPLC. The desired fraction was collected and concentrated to give phenyl (4-cyano-3,5-dimethylphenyl)carbamate. ¹H NMR (400 MHz, DMSO-d₆) δ=10.59 (s, 1H), 7.50-7.41 (m, 3H), 7.39 (s, 2H), 7.27-7.22 (m, 2H), 2.42 (s, 6H). MS (ESI) m/z 267.1 [M+H]⁺

Step 3: To a solution of 3-(6-(hydroxymethyl)-1-oxoisoindolin-2-yl)piperidine-2,6-dione VIII (90.0 mg, 328 umol, 1.00 eq) in dimethyl formamide (2.00 mL) was added phenyl (4-cyano-3,5-dimethylphenyl)carbamate (105 mg, 394 umol, 1.20 eq) and sodium hydride (26.2 mg, 656 umol, 60% purity, 2.00 eq) at 10° C. The mixture was stirred at 10° C. for 1 h. The mixture was adjusted pH=6 with formic acid (0.100 mL). The mixture was filtered to give filtrate. The filtrate was purified by prep-HPLC and the desired fraction was collected and lyophilized to give (2-(2,6-dioxopiperidin-3-yl)-3-oxoisoindolin-5-yl)methyl(4-cyano-3,5-dimethylphenyl)carbamate 108. ¹H NMR (400 MHz, DMSO-d₆) δ=10.98 (s, 1H), 10.18 (s, 1H), 7.80 (s, 1H), 7.72-7.66 (m, 1H), 7.66-7.60 (m, 1H), 7.36 (s, 2H), 5.29 (s, 2H), 5.12 (dd, J=5.1, 13.3 Hz, 1H), 4.53-4.43 (m, 1H), 4.39-4.31 (m, 1H), 2.97-2.85 (m, 1H), 2.60 (br dd, J=1.8, 15.7 Hz, 1H), 2.44-2.37 (m, 7H), 2.05-1.96 (m, 1H). MS (ESI) m/z 447.2 [M+H]⁺

### Compound 309:

Step 1: To a solution of 1-(5-bromopyridin-2-yl)ethanone (8.00 g, 40.0 mmol, 1.00 eq) in dichloromethane (100 mL) was added triethylamine (12.4 g, 122 mmol, 17.0 mL, 3.05 eq) and chlorotrimethylsilane (8.56 g, 78.8 mmol, 10.0 mL, 1.97 eq). The mixture was stirred at 5° C. for 12 h. The mixture was concentrated under reduced pressure. The residue was purified by column chromatography on silica gel to give 5-bromo-2-(1-((trimethylsilyl)oxy)vinyl)pyridine.

Step 2: To a solution of 5-bromo-2-(1-((trimethylsilyl)oxy)vinyl)pyridine (4.50 g, 16.5 mmol, 1.00 eq) (crude) in dichloromethane (100 mL) was added diiodomethane (6.64 g, 24.8 mmol, 2.00 mL, 1.50 eq) and diethylzinc (1 M in toluene, 25.0 mL, 1.51 eq) at 0° C. The mixture was stirred at 5° C. for 6 h under nitrogen. The mixture was quenched with water (10.0 mL) and filtered. The organic layer was separated and concentrated under reduced pressure. The residue was purified by column chromatography on silica gel to give 5-bromo-2-(1-((trimethylsilyl)oxy)cyclopropyl) pyridine. ¹H NMR (400 MHz, CDCl₃) δ=8.59 (d, J=2.3 Hz, 1H), 7.80 (dd, J=2.3, 8.4 Hz, 1H), 7.50 (d, J=8.5 Hz, 1H), 1.36-1.17 (m, 2H), 0.92-0.83 (m, 2H), 0.31-0.29 (m, 9H).

Step 3: To a solution of 5-bromo-2-(1-((trimethylsilyl)oxy)cyclopropyl)pyridine (360 mg, 1.26 mmol, 1.00 eq) in dioxane (10.0 mL) was added tert-butyl carbamate (350 mg, 2.99 mmol, 2.38 eq), cesium carbonate (1.23 g, 3.77 mmol, 2.99 eq) and methanesulfonato(2-dicyclohexylphosphino-2,6-di-i-propoxy-1,1-biphenyl)(2-amino-1,1-biphenyl-2-yl)palladium(II) (120 mg, 143 umol, 0.114 eq). The mixture was stirred at 90° C. for 2 h under nitrogen. The mixture was diluted with ethyl acetate (50.0 mL) and water (50.0 mL). The organic layer was separated and concentrated under reduced pressure. The residue was purified by column chromatography on silica gel to give tert-butyl (6-(1-((trimethylsilyl)oxy)cyclopropyl)pyridin-3-yl)carbamate. ¹H NMR (400 MHz, CDCl₃) δ=8.25 (d, J=2.4 Hz, 1H), 7.55 (d, J=8.5 Hz, 1H), 7.49-7.39 (m, 1H), 6.52 (br s, 1H), 1.46 (s, 9H), 1.37-1.32 (m, 2H), 1.22-1.18 (m, 2H), 0.19-0.09 (m, 9H).

Step 4: To a solution of tert-butyl (6-(1-((trimethylsilyl)oxy)cyclopropyl)pyridin-3-yl)carbamate (320 mg, 992 umol, 1.00 eq) in dichloromethane (50.0 mL) was added diethylaminosulfurtrifluoride (244 mg, 1.51 mmol, 200 uL, 1.53 eq) at −30° C. The mixture was stirred at −30° C. for 0.5 h. The mixture was quenched with saturated sodium bicarbonate (10%, 10.0 mL). The organic layer was separated and concentrated under reduced pressure. The residue was purified by prep-TLC to give tert-butyl (6-(1-fluorocyclopropyl)pyridin-3-yl)carbamate. ¹H NMR (400 MHz, CDCl₃) δ=8.30 (d, J=2.4 Hz, 1H), 7.97 (br s, 1H), 7.55 (d, J=8.4 Hz, 1H), 6.50 (br s, 1H), 1.53 (s, 9H), 1.49-1.41 (m, 2H), 1.39-1.35 (m, 2H). MS (ESI) m/z 253.1 [M+H]⁺

Step 5: To a solution tert-butyl (6-(1-fluorocyclopropyl)pyridin-3-yl)carbamate (25.0 mg, 99.1 umol, 1.00 eq) in ethyl acetate (5.00 mL) was added hydrochloric acid/ethyl acetate (4 M, 2.00 mL) in portions. The mixture was stirred at 10° C. for 3 h. The reaction mixture was concentrated under reduced pressure to give 6-(1-fluorocyclopropyl)pyridin-3-amine. MS (ESI) m/z 153.1 [M+H]⁺

Step 6: To a solution of 6-(1-fluorocyclopropyl)pyridin-3-amine (20.0 mg, 131 umol, 1.00 eq) in Acetonitrile (2.00 mL) was added pyridine (31.2 mg, 394 umol, 31.8 uL, 3.00 eq) and phenyl carbonochloridate (24.6 mg, 157 umol, 19.7 uL, 1.20 eq). The mixture was stirred at 10° C. for 1 h and concentrated to give a residue, which was purified by prep-TLC to give pyridin-3-yl (6-(1-fluorocyclopropyl)pyridin-3-yl)carbamate. MS (ESI) m/z 273.0 [M+H]⁺

Step 7: To a solution of 3-(6-(hydroxymethyl)-1-oxoisoindolin-2-yl)piperidine-2,6-dione VIII (13.0 mg, 47.4 umol, 1.30 eq) in dimethyl formamide (1.00 mL) was added pyridin-3-yl (6-(1-fluorocyclopropyl)pyridin-3-yl) carbamate (9.96 mg, 36.5 umol, 1.00 eq) and sodium hydride (2.92 mg, 72.9 umol, 60% purity, 2.00 eq). The mixture was stirred at 0° C. for 1 h. The mixture was adjusted pH=6 with Formic acid (0.100 mL). The mixture was diluted with water/ethyl acetate (2.00 ml/2.00 ml). The organic layer was collected and concentrated to give a residue. The residue was purified by prep-HPLC and the desired fraction was collected and lyophilized to give (2-(2,6-dioxopiperidin-3-yl)-3-oxoisoindolin-5-yl)methyl (6-(1-fluorocyclopropyl)pyridin-3-yl)carbamate 109. ¹H NMR (400 MHz, DMSO-d₆) δ=11.14-10.79 (m, 1H), 10.07 (br s, 1H), 8.56 (s, 1H), 8.00-7.90 (m, 1H), 7.80 (s, 1H), 7.74-7.66 (m, 1H), 7.66-7.60 (m, 1H), 7.53 (d, J=7.7 Hz, 1H), 5.29 (s, 2H), 5.12 (dd, J=5.1, 13.3 Hz, 1H), 4.51-4.42 (m, 1H), 4.39-4.29 (m, 1H), 2.96-2.86 (m, 1H), 2.62-2.58 (m, 1H), 2.45-2.34 (m, 1H), 2.06-1.96 (m, 1H), 1.49-1.39 (m, 2H), 1.30-1.22 (m, 2H). MS (ESI) m/z 453.2 [M+H]⁺

### Compound 310:

Step 1: A mixture of 5-amino-2-methylbenzonitrile (1.00 g, 7.57 mmol, 1.00 eq), pyridine (1.80 g, 22.7 mmol, 1.83 mL, 3.00 eq) in Acetonitrile (20.0 mL) was added phenyl carbonochloridate (1.30 g, 8.32 mmol, 1.04 mL, 1.10 eq) at 0° C. The mixture was stirred at 25° C. for 1 h. The reaction mixture was concentrated under reduced pressure to give a residue. The residue was purified by reversed-phase HPLC and lyophilized to give phenyl (3-cyano-4-methylphenyl)carbamate. MS (ESI) m/z. 253.1 [M+H]⁺

Step 2: To a solution of phenyl (3-cyano-4-methylphenyl)carbamate (80.9 mg, 321 umol, 1.10 eq) and 3-(6-(hydroxymethyl)-1-oxoisoindolin-2-yl)piperidine-2,6-dione VIII (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 25° C. for 1 h. The pH of the mixture was adjusted to around 6 by adding hydrochloric acid. The mixture was extracted with ethyl acetate (3×10.0 mL). The combined organic layers were dried over anhydrous sodium sulfate, sodium sulfate, filtered and concentrated under reduced pressure to give a residue. The residue was purified by prep-HPLC and lyophilized to afford (2-(2,6-dioxopiperidin-3-yl)-3-oxoisoindolin-5-yl)methyl(3-cyano-4-methylphenyl)carbamate 110. ¹H NMR (400 MHz, DMSO-d₆) δ=10.99 (s, 1H), 10.09 (s, 1H), 7.81 (d, J=2.0 Hz, 1H), 7.80 (s, 1H), 7.70-7.67 (m, 1H), 7.65-7.60 (m, 2H), 7.38 (d, J=8.6 Hz, 1H), 5.29 (s, 2H), 5.12 (dd, J=5.0, 13.3 Hz, 1H), 4.52-4.44 (m, 1H), 4.37-4.30 (m, 1H), 2.96-2.86 (m, 1H), 2.60 (br d, J=17.9 Hz, 1H), 2.40 (s, 3H), 2.35 (br d, J=3.8 Hz, 1H), 2.05-1.97 (m, 1H). MS (ESI) m/z. 433.0[M+H]⁺

### Compound 311:

Step 1: To a solution of 3-amino-5-chlorobenzonitrile (0.500 g, 3.28 mmol, 1.00 eq) in Acetonitrile (10.0 mL) was added pyridine (1.30 g, 16.3 mmol, 1.32 mL, 5.00 eq) and phenyl carbonochloridate (564 mg, 3.60 mmol, 451 uL, 1.10 eq). The mixture was stirred at 10° C. for 0.5 h. The reaction mixture was concentrated under reduced pressure to give a residue. The crude product was purified by reversed-phase HPLC to give phenyl (3-chloro-5-cyanophenyl)carbamate. ¹H NMR (400 MHz, DMSO-d₆) δ=10.81 (br s, 1H), 7.94-7.81 (m, 3H), 7.48-7.44 (m, 2H), 7.32-7.25 (m, 3H).

Step 2: To a solution of 3-(6-(hydroxymethyl)-1-oxoisoindolin-2-yl)piperidine-2,6-dione VIII (80.0 mg, 291 umol, 1.00 eq) in dimethyl formamide (2.00 mL) was added phenyl (3-chloro-5-cyanophenyl)carbamate (87.4 mg, 320 umol, 1.10 eq) and sodium hydride (23.3 mg, 583 umol, 60% purity, 2.00 eq) at 0° C. The mixture was stirred at 0° C. for 20 min. The reaction mixture was quenched with Formic acid (1.00 ml) to give a solution. The solution was purified by prep-HPLC and lyophilized to give (2-(2,6-dioxopiperidin-3-yl)-3-oxoisoindolin-5-yl)methyl (3-chloro-5-cyanophenyl)carbamate 111. ¹H NMR (400 MHz, DMSO-d₆) δ=11.00 (br s, 1H), 10.41 (br s, 1H), 7.86 (t, J=1.9 Hz, 1H), 7.81 (dd, J=2.3, 4.0 Hz, 2H), 7.72-7.69 (m, 1H), 7.69-7.63 (m, 2H), 5.32 (s, 2H), 5.13 (dd, J=5.1, 13.3 Hz, 1H), 4.53-4.45 (m, 1H), 4.39-4.30 (m, 1H), 2.97-2.87 (m, 1H), 2.65-2.61 (m, 1H), 2.46-2.38 (m, 1H), 2.06-1.98 (m, 1H). MS (ESI) m/z 453.1 [M+H]⁺

### Compound 312:

Step 1: To a solution of 3-methyl-5-nitrobenzonitrile (1.00 g, 6.17 mmol, 1.00 eq) in the mixture of methanol (10.0 mL) and water (5.00 mL) was added iron powder (1.72 g, 30.8 mmol, 5.00 eq) and ammonium chloride (2.64 g, 49.3 mmol, 8.00 eq). The mixture was stirred at 80° C. for 2 h. The mixture was filtered to give filter liquor and concentrated under reduced pressure to give a residue. The crude product was diluted with water (30.0 mL) and exacted with ethyl acetate (2×50.0 mL). The organic phase was separated, washed with brine, dried over sodium sulfate, filtered and concentrated under reduced pressure to give 3-amino-5-methylbenzonitrile. ¹H NMR (400 MHz, DMSO-d₆) δ=6.68 (brd, J=8.8 Hz, 3H), 5.51 (br s, 2H), 2.19 (br s, 3H).

Step 2: To a solution of 3-amino-5-methylbenzonitrile (750 mg, 5.67 mmol, 1.00 eq) in Acetonitrile (10.0 mL) was added pyridine (2.24 g, 28.3 mmol, 2.29 mL, 5.00 eq) and phenyl carbonochloridate (977 mg, 6.24 mmol, 781 uL, 1.10 eq). The mixture was stirred at 10° C. for 0.5 h. The reaction mixture was concentrated under reduced pressure to give a residue. The crude product was purified by reversed-phase HPLC to give phenyl (3-cyano-5-methylphenyl)carbamate. MS (ESI) m/z 253.0 [M+H]⁺

Step 3: To a solution of 3-(6-(hydroxymethyl)-1-oxoisoindolin-2-yl)piperidine-2,6-dione VIII (80.0 mg, 291 umol, 1.00 eq) in dimethyl formamide (2.00 mL) was added phenyl (3-cyano-5-methylphenyl)carbamate (80.9 mg, 320 umol, 1.10 eq) and sodium hydride (23.3 mg, 583 umol, 60% purity, 2.00 eq) at 0° C. The mixture was stirred at 0° C. for 20 min. The reaction mixture was quenched with Formic acid (1.00 ml) to give a solution. The solution was purified by prep-HPLC and lyophilized to give (2-(2,6-dioxopiperidin-3-yl)-3-oxoisoindolin-5-yl)methyl (3-cyano-5-methylphenyl)carbamate 112. ¹H NMR (400 MHz, DMSO-d₆) δ=11.00 (s, 1H), 10.15 (s, 1H), 7.81 (s, 1H), 7.73-7.67 (m, 2H), 7.67-7.63 (m, 1H), 7.58 (s, 1H), 7.31 (s, 1H), 5.30 (s, 2H), 5.13 (dd, J=5.1, 13.3 Hz, 1H), 4.51-4.43 (m, 1H), 4.39-4.31 (m, 1H), 2.98-2.86 (m, 1H), 2.61 (br dd, J=2.4, 15.3 Hz, 1H), 2.41 (dd, J=4.5, 13.1 Hz, 1H), 2.32 (s, 3H), 2.05-1.96 (m, 1H). MS (ESI) m/z 433.2 [M+H]⁺

### Compound 313:

Step 1: A mixture of sodium hydroxide (12.0 g, 299 mmol, 10.0 eq) and dimethyl formamide (10.0 mL) was stirred at 95° C. for 0.5 h, then 2-hydroxy-5-nitrobenzaldehyde (5.00 g, 29.9 mmol, 1.00 eq) and sodium; 2-chloro-2,2-difluoroacetate (23.0 g, 151 mmol, 5.04 eq) in dimethyl formamide (30.0 mL) was slowly added to the mixture was stirred at 95° C. for another 1.5 h. After being cooled to room temperature, the reaction mixture was diluted with water (250 mL) and extracted with ethyl acetate (300 mL). The organic layer was collected and dried over anhydrous sodium sulfate. After filtration, the filtrate was concentrated under reduced pressure to give a residue, which was purified by column chromatography on silica gel and further purified by reversed-phase HPLC. The desired fraction was collected and lyophilized to give 2,2-difluoro-5-nitro-2,3-dihydrobenzofuran-3-ol. ¹H NMR (400 MHz, CDCl₃) δ=8.39 (d, J=2.1 Hz, 1H), 8.35 (dd, J=2.3, 8.8 Hz, 1H), 7.13 (d, J=8.9 Hz, 1H), 5.40 (dt, J=4.2, 8.9 Hz, 1H), 2.59 (dd, J=1.8, 8.2 Hz, 1H).

Step 2: To a solution of 2,2-difluoro-5-nitro-2,3-dihydrobenzofuran-3-ol (1.00 g, 4.61 mmol, 1.00 eq) in tetrahydrofuran (15.0 mL) was added sodium hydride (60%, dispersion in paraffin liquid) (221 mg, 5.53 mmol, 60% purity, 1.20 eq) and 4-methylbenzene-1-sulfonyl chloride (1.76 g, 9.21 mmol, 2.00 eq) in portions. The mixture was stirred at 10° C. for 3 h, sodium hydride (60%, dispersion in paraffin liquid) (270 mg, 6.75 mmol, 60% purity, 1.47 eq) was added into the mixture. The mixture was stirred at 0° C. for 0.5 h, then 4-methylbenzene-1-sulfonyl chloride (1.05 g, 5.53 mmol, 1.20 eq) was added into the reaction mixture was stirred at 0° C. for another 1.5 h. The reaction mixture was adjusted pH=8 with formic acid and diluted with ethyl acetate. The organic layer was separated and concentrated under reduced pressure to give a crude product. The crude product was purified by column chromatography on silica gel to give 2,2-difluoro-5-nitro-2,3-dihydrobenzofuran-3-yl 4-methylbenzenesulfonate. ¹H NMR (400 MHz, CDCl₃) δ=8.36 (dd, J=2.3, 8.9 Hz, 1H), 8.00 (d, J=2.2 Hz, 1H), 7.91 (d, J=8.2 Hz, 2H), 7.46 (d, J=8.3 Hz, 2H), 7.12 (d, J=9.0 Hz, 1H), 5.96 (dd, J=2.1, 9.2 Hz, 1H), 2.53 (s, 3H).

Step 3: To a solution of 2,2-difluoro-5-nitro-2,3-dihydrobenzofuran-3-yl 4-methylbenzenesulfonate (740 mg, 1.99 mmol, 1.00 eq) in ethyl acetate (20.0 mL) and tetrahydrofuran (20.0 mL) was added palladium on activated carbon (10%) (150 mg, wet) in portions under hydrogen (15.0 Psi). The mixture was stirred at 10° C. for 30 min. The reaction mixture was filtered to give the filtrates and concentrated under reduced pressure to give a crude product. The crude product purified by column chromatography on silica gel to give 2,2-difluoro-2,3-dihydrobenzofuran-5-amine. ¹H NMR (400 MHz, DMSO-d₆) δ=7.47 (d, J=8.0 Hz, 1H), 7.11 (d, J=7.9 Hz, 1H), 6.79 (d, J=8.5 Hz, 1H), 6.65 (s, 1H), 6.58-6.54 (m, 1H), 3.68 (t, J=14.7 Hz, 2H). MS (ESI) m/z 172.2 [M+H]⁺

Step 4: To a solution of 2,2-difluoro-2,3-dihydrobenzofuran-5-amine (76.0 mg, 444 umol, 1.00 eq) in Acetonitrile (5.00 mL) was added pyridine (106 mg, 1.34 mmol, 108 uL, 3.01 eq) and phenyl carbonochloridate (82.5 mg, 527 umol, 66.0 uL, 1.19 eq) in portions at 0° C. The mixture was stirred at 10° C. for 0.5 h. The reaction mixture was concentrated under reduced pressure to give a residue. The crude product was purified by column chromatography on silica gel to give phenyl (2,2-difluoro-2,3-dihydrobenzofuran-5-yl)carbamate. ¹H NMR (400 MHz, CDCl₃) δ=7.59-7.50 (m, 1H), 7.43-7.39 (m, 2H), 7.26-7.15 (m, 4H), 6.93 (d, J=8.7 Hz, 1H), 6.90-6.79 (m, 1H), 3.61 (t, J=13.9 Hz, 2H). MS (ESI) m/z 291.9 [M+H]⁺

Step 5: To a solution of 3-(6-(hydroxymethyl)-1-oxoisoindolin-2-yl)piperidine-2,6-dione VIII (20.0 mg, 72.9 umol, 1.00 eq) and phenyl (2,2-difluoro-2,3-dihydrobenzofuran-5-yl)carbamate (25.5 mg, 87.5 umol, 1.20 eq) in dimethyl formamide (2.00 mL) was added triethylamine (29.1 mg, 287 umol, 40.0 uL, 3.94 eq) in portions. The mixture was stirred at 25° C. for 12 h. The mixture was diluted with water (10.0 mL) and extracted with ethyl acetate (20.0 mL). The combined organic layer was dried over anhydrous sodium sulfate. After filtration, the filtrate was concentrated under reduced pressure to give a residue. The crude product was purified by column chromatography on silica gel to give a residue. The residue was dissolved in acetonitrile/dimethyl formamide (1/1, 2.00 mL) and purified by prep-HPLC. The desired fraction was collected and concentrated under pressure to give a solution, which was lyophilized to give (2-(2,6-dioxopiperidin-3-yl)-3-oxoisoindolin-5-yl)methyl(2,2-difluoro-2,3-dihydrobenzofuran-5-yl)carbamate 113. ¹H NMR (400 MHz, DMSO-d₆) δ=10.99 (br s, 1H), 9.83 (br s, 1H), 7.79 (s, 1H), 7.70-7.66 (m, 1H), 7.65-7.60 (m, 1H), 7.52 (br s, 1H), 7.32 (br d, J=8.3 Hz, 1H), 7.01 (d, J=8.8 Hz, 1H), 5.26 (s, 2H), 5.12 (dd, J=5.0, 13.3 Hz, 1H), 4.50-4.44 (m, 1H), 4.37-4.30 (m, 1H), 3.80 (t, J=14.7 Hz, 2H), 2.97-2.86 (m, 1H), 2.62-2.57 (m, 1H), 2.43-2.34 (m, 1H), 2.04-1.98 (m, 1H). MS (ESI) m/z 472.0 [M+H]⁺

### Compound 314:

Step 1: To a solution of 2-(tert-butyl)isonicotinic acid (150 mg, 837 umol, 1.00 eq) in dioxane (1.00 mL) was added diphenylphosphoryl azide (461 mg, 1.67 mmol, 363 uL, 2.00 eq) and triethylamine (254 mg, 2.51 mmol, 349 uL, 3.00 eq) at 25° C. The mixture was stirred at 25° C. for 0.5 h under nitrogen, then added 3-(6-(hydroxymethyl)-1-oxoisoindolin-2-yl)piperidine-2,6-dione VIII (230 mg, 837 umol, 1.00 eq). The mixture was stirred at 90° C. for 12 h under nitrogen. The reaction mixture was added hydrochloric acid (1 M, 2.00 mL) and filtered to give a filter cake. The filter cake was purified twice by Prep-HPLC to give (2-(2,6-dioxopiperidin-3-yl)-3-oxoisoindolin-5-yl)methyl(2-(tert-butyl)pyridin-4-yl)carbamate 144. ¹H NMR (400 MHz, DMSO-d₆) δ=11.00 (br s, 1H), 10.18 (s, 1H), 8.34 (d, J=5.6 Hz, 1H), 7.82 (s, 1H), 7.72-7.68 (m, 1H), 7.68-7.64 (m, 1H), 7.52 (d, J=1.6 Hz, 1H), 7.28 (dd, J=2.0, 5.6 Hz, 1H), 5.32 (s, 2H), 5.14 (dd, J=5.2, 13.2 Hz, 1H), 4.54-4.44 (m, 1H), 4.38-4.32 (m, 1H), 2.98-2.88 (m, 1H), 2.64-2.58 (m, 1H), 2.44-2.38 (m, 1H), 2.06-1.98 (m, 1H), 1.28 (s, 9H). MS (ESI) m/z 451.2[M+H]⁺

### Compound 315:

Step 1: Triethylamine (9.61 g, 94.9 mmol, 13.2 mL, 2.10 eq) and chloro(trimethyl)silane (10.3 g, 94.9 mmol, 12.1 mL, 2.10 eq) were added into a stirred solution of 1-(4-bromophenyl)ethanone (9.00 g, 45.2 mmol, 1.00 eq) in Acetonitrile (100 mL) at 0° C. The mixture was stirred at 25° C. for 16 h under nitrogen atmosphere. The reaction mixture was quenched by addition saturated ammonium chloride (500 mL) at 0° C., and extracted with ethyl acetate (3×500 mL). The combined organic layers were dried over anhydrous sodium sulfate, filtered and concentrated under reduced pressure to give a residue. The residue was purified by column chromatography to give ((1-(4-bromophenyl)vinyl)oxy) trimethylsilane. ¹H NMR (400 MHz, CDCl₃) δ=7.46 (s, 4H), 4.91 (d, J=1.9 Hz, 1H), 4.45 (d, J=1.9 Hz, 1H), 0.31-0.24 (m, 9H).

Step 2: A mixture of ((1-(4-bromophenyl)vinyl)oxy)trimethylsilane (6.50 g, 23.9 mmol, 1.00 eq), diiodomethane (9.63 g, 35.9 mmol, 2.90 mL, 1.50 eq) in dichloromethane (50.0 mL) was degassed and purged with nitrogen for 3 times, and then diethylzinc (1 M in toluene, 36.0 mL, 1.50 eq) was added into the mixture at 0° C. The mixture was stirred at 20° C. for 12 h under nitrogen atmosphere. The reaction mixture was quenched by addition water (100 mL), and extracted with ethyl acetate (2×100 mL). The combined organic layers were dried over anhydrous sodium sulfate, filtered and concentrated under reduced pressure to give a residue. The residue was purified by column chromatography to give (1-(4-bromophenyl)cyclopropoxy)trimethylsilane. ¹H NMR (400 MHz, CDCl₃) δ=7.43 (d, J=8.4 Hz, 2H), 7.15 (d, J=8.5 Hz, 2H), 1.26-1.22 (m, 2H), 1.01-0.97 (m, 2H), 0.09 (s, 9H).

Step 3: A mixture of (1-(4-bromophenyl)cyclopropoxy)trimethylsilane (200 mg, 701 umol, 1.00 eq), tert-butyl carbamate (164 mg, 1.40 mmol, 2.00 eq), and cesium carbonate (685 mg, 2.10 mmol, 3.00 eq) in dioxane (10.0 mL) was degassed and purged with nitrogen for 3 times, and then palladium(1+)2-amino-1,1-biphenyl-2-yl[2,6-bis(propan-2-yloxy)-[1,1-biphenyl]-2-yl]dicyclohexylphosphane methanesulfonate (58.6 mg, 70.1 umol, 0.100 eq) was added into the mixture. The mixture was stirred at 90° C. for 6 h under nitrogen atmosphere. The reaction mixture was diluted with water (100 mL), and then extracted with ethyl acetate (3×150 mL). The combined organic layers were dried over anhydrous sodium sulfate, filtered and concentrated under reduced pressure to give a residue. The residue was purified by flash silica gel chromatography to give tert-butyl (4-(1-((trimethylsilyl)oxy)cyclopropyl)phenyl)carbamate. ¹H NMR (400 MHz, CDCl₃) δ=7.34-7.27 (m, 2H), 7.24-7.20 (m, 2H), 6.44 (br s, 1H), 1.52 (s, 9H), 1.19-1.15 (m, 2H), 0.99-0.93 (m, 2H), 0.06 (s, 9H).

Step 4: A mixture of tert-butyl (4-(1-((trimethylsilyl)oxy)cyclopropyl)phenyl)carbamate (490 mg, 1.52 mmol, 1.00 eq), diethylaminosulfurtrifluoride (491 mg, 3.05 mmol, 403 uL, 2.00 eq) in dichloromethane (10.0 mL) was stirred at −30° C. for 1 h under nitrogen. The reaction mixture was concentrated under reduced pressure to give a residue. The residue was purified by column chromatography to give tert-butyl (4-(1-fluorocyclopropyl)phenyl)carbamate. ¹H NMR (400 MHz, CDCl₃) δ=7.36 (br d, J=8.4 Hz, 2H), 7.24 (d, J=8.3 Hz, 2H), 6.50 (s, 1H), 1.53 (s, 9H), 1.48-1.44 (m, 1H), 1.43-1.39 (m, 1H), 1.04-0.98 (m, 2H). MS (ESI) m/z 196.0 [M+H−56]⁺

Step 5: To a solution of tert-butyl (4-(1-fluorocyclopropyl)phenyl)carbamate (150 mg, 597 umol, 1.00 eq) in ethyl acetate (5.00 mL) was added hydrochloric acid/ethyl acetate (4 M, 5.00 mL). The mixture was stirred at 10° C. for 2 h. The reaction mixture was concentrated under reduced pressure to give a residue to give 4-(1-fluorocyclopropyl)aniline. ¹H NMR (400 MHz, DMSO-d₆) δ=10.50-9.62 (m, 2H), 7.40-7.28 (m, 4H), 1.53-1.43 (m, 2H), 1.19-1.11 (m, 2H).

Step 6: A mixture of 4-(1-fluorocyclopropyl)aniline (120 mg, 639 umol, 1.00 eq, hydrochloric acid), phenyl carbonochloridate (110 mg, 703 umol, 88.1 uL, 1.10 eq), pyridine (152 mg, 1.92 mmol, 154 uL, 3.00 eq) in Acetonitrile (10.0 mL) was stirred at 0° C. The mixture was stirred at 25° C. for 1 h. The mixture was concentrated under reduced pressure to give a residue. The residue was purified by column chromatography to give phenyl (4-(1-fluorocyclopropyl)phenyl)carbamate. ¹H NMR (400 MHz, DMSO-d₆) δ=10.29 (br s, 1H), 7.53 (d, J=8.4 Hz, 2H), 7.46-7.40 (m, 2H), 7.29-7.20 (m, 5H), 1.46-1.37 (m, 2H), 1.13-1.05 (m, 2H). MS (ESI) m/z. 272.1 [M+H]⁺

Step 7: To a solution of phenyl (4-(1-fluorocyclopropyl)phenyl)carbamate (90.0 mg, 332 umol, 1.00 eq), 3-(6-(hydroxymethyl)-1-oxoisoindolin-2-yl)piperidine-2,6-dione VIII (100 mg, 365 umol, 1.10 eq) in dimethyl formamide (1.50 mL) was added sodium hydride (26.5 mg, 664 umol, 60% purity, 2.00 eq). The mixture was stirred at 0° C. for 1 h. The pH of the mixture was adjusted to around 6 with Formic acid. The mixture was diluted with water (50.0 mL) and extracted with ethyl acetate (3×50.0 mL). The combined organic layers were dried over anhydrous sodium sulfate, filtered and concentrated under reduced pressure to give a residue. The residue was purified by column chromatography to afford (2-(2,6-dioxopiperidin-3-yl)-3-oxoisoindolin-5-yl)methyl(4-(1-fluorocyclopropyl)phenyl) carbamate 115. ¹H NMR (400 MHz, DMSO-d₆) δ=10.98 (s, 1H), 9.88 (s, 1H), 7.80 (s, 1H), 7.70-7.62 (m, 2H), 7.49 (br d, J=8.3 Hz, 2H), 7.24 (d, J=8.7 Hz, 2H), 5.27 (s, 2H), 5.12 (dd, J=5.1, 13.3 Hz, 1H), 4.50-4.43 (m, 1H), 4.37-4.30 (m, 1H), 2.96-2.86 (m, 1H), 2.63-2.58 (m, 1H), 2.40 (dd, J=4.4, 13.3 Hz, 1H), 2.05-1.99 (m, 1H), 1.45-1.34 (m, 2H), 1.11-1.02 (m, 2H). MS (ESI) m/z. 903.2 [2M+H]⁺

### Compound 316:

Step 1: To a solution of diethyl malonate (1.86 g, 11.6 mmol, 1.76 mL, 1.50 eq) in tetrahydrofuran (20.0 mL) was added sodium hydride (619 mg, 15.5 mmol, 60% purity, 2.00 eq) at 0° C. The mixture was stirred at 0° C. for 30 min, then 1-chloro-2,3-difluoro-5-nitrobenzene (1.50 g, 7.75 mmol, 1.00 eq) was added to the mixture and stirred at 25° C. for another 11.5 h. The mixture was quenched with saturated ammonium chloride solution and extracted with ethyl acetate (3×30.0 mL). The combined organic phase was washed with brine (50.0 mL), dried with anhydrous sodium sulfate, filtered and concentrated in vacuum to give a residue. The residue was purified by silica gel chromatography to give diethyl 2-(2-chloro-6-fluoro-4-nitrophenyl)malonate I. ¹H NMR (400 MHz, CDCl₃) δ=8.17 (t, J=1.6 Hz, 1H), 7.94 (dd, J=2.4, 9.2 Hz, 1H), 5.23 (s, 1H), 4.30 (q, J=7.2 Hz, 4H), 1.33-1.31 (m, 6H). MS (ESI) m/z 334.0 [M+H]⁺

Step 2: A mixture of diethyl 2-(2-chloro-6-fluoro-4-nitrophenyl)malonate (1.00 g, 3.00 mmol, 1.00 eq) and magnesium chloride (855 mg, 8.99 mmol, 369 uL, 3.00 eq) in dimethylacetamide (10.0 mL) and water (647 mg, 35.9 mmol, 647 uL, 12.0 eq) was stirred at 140° C. for 5 h. The reaction mixture was poured into water (50.0 mL) and extracted with ethyl acetate (3×30.0 mL). The combined organic layers were washed with brine (50.0 mL), dried over anhydrous sodium sulfate, filtered and concentrated in vacuum to give a residue. The residue was purified by silica gel chromatography to give 1-chloro-3-fluoro-2-methyl-5-nitrobenzene. ¹H NMR (400 MHz, CDCl₃) δ=8.11 (s, 1H), 7.86 (dd, J=2.0, 8.8 Hz, 1H), 2.43 (d, J=2.4 Hz, 3H).

Step 3: To a mixture of 1-chloro-3-fluoro-2-methyl-5-nitrobenzene (80.0 mg, 422 umol, 1.00 eq), iron powder (118 mg, 2.11 mmol, 5.00 eq) and ammonium chloride (112 mg, 2.11 mmol, 5.00 eq) in methanol (2.00 mL) and water (500 uL) was stirred at 80° C. for 2 h. The reaction mixture was concentrated to give a residue. The residue was purified by reversed-phase HPLC and lyophilized to give 3-chloro-5-fluoro-4-methylaniline. MS (ESI) m/z 160.1 [M+H]⁺

Step 4: To a solution of 3-chloro-5-fluoro-4-methylaniline (60.0 mg, 375 umol, 1.00 eq) and pyridine (89.2 mg, 1.13 mmol, 91.0 uL, 3.00 eq) in Acetonitrile (1.00 mL) was added phenyl carbonochloridate (70.6 mg, 451 umol, 56.5 uL, 1.20 eq) at 25° C. The reaction mixture was stirred at 25° C. for 2 h. The reaction mixture was concentrated to give a residue, which was purified by reversed-phase HPLC and lyophilized to give phenyl (3-chloro-5-fluoro-4-methylphenyl)carbamate. MS (ESI) m/z 280.0 [M+H]⁺

Step 5: To a mixture of 3-(6-(hydroxymethyl)-1-oxoisoindolin-2-yl)piperidine-2,6-dione VIII (78.4 mg, 286 umol, 1.00 eq) and phenyl (3-chloro-5-fluoro-4-methylphenyl)carbamate (80.0 mg, 286 umol, 1.00 eq) in dimethyl formamide (2.00 mL) was added sodium hydride (11.4 mg, 286 umol, 60% purity, 1.00 eq) at 0° C. The reaction mixture was stirred at 25° C. for 1 h. The reaction mixture was added Formic acid (1.00 mL) and filtered to give a filtrate. The filtrate was purified by Prep-HPLC and lyophilized to give (2-(2,7-dioxoazepan-3-yl)-3-oxoisoindolin-5-yl)methyl (3-chloro-4-methylphenyl) carbamate 116. ¹H NMR (400 MHz, DMSO-d₆) δ=11.07-10.93 (m, 1H), 10.14 (s, 1H), 7.80 (s, 1H), 7.72-7.67 (m, 1H), 7.67-7.63 (m, 1H), 7.40 (s, 1H), 7.34-7.29 (m, 1H), 5.29 (s, 2H), 5.13 (dd, J=5.2, 13.2 Hz, 1H), 4.52-4.44 (m, 1H), 4.39-4.31 (m, 1H), 2.92 (ddd, J=5.6, 13.6, 17.6 Hz, 1H), 2.64-2.58 (m, 1H), 2.43 (dt, J=4.4, 13.2 Hz, 1H), 2.18 (d, J=1.6 Hz, 3H), 2.07-1.97 (m, 1H). MS (ESI) m/z 460.3 [M+H]⁺.

### Compound 317:

Step 1: To a solution of 3-fluoro-5-methylaniline (2.00 g, 15.9 mmol, 1.00 eq) in dimethyl formamide (10.0 mL) was added N-bromosuccinimide (2.87 g, 16.1 mmol, 1.01 eq) in portions at 0° C. The mixture was stirred at 20° C. for 1 h. The mixture was diluted with water (180 mL) and extracted with ethyl acetate (3×50.0 mL). The combined organic layer was washed with brine and dried over anhydrous sodium sulfate, filtered and concentrated to give 4-bromo-3-fluoro-5-methylaniline. ¹H NMR (400 MHz, DMSO-d₆) δ=6.37 (s, 1H), 6.32 (dd, J=2.5, 11.3 Hz, 1H), 5.47 (br s, 2H), 2.22 (s, 3H).

Step 2: To a solution of 4-bromo-3-fluoro-5-methylaniline (1.00 g, 4.90 mmol, 1.00 eq), potassium methyltrifluoroborate (2.00 g, 16.4 mmol, 3.35 eq) and potassium carbonate (2.03 g, 14.7 mmol, 3.00 eq) in dioxane (10.0 mL) and water (2.00 mL) was added [1,1′-bis(diphenylphosphino)ferrocene] dichloropalladium(II) (400 mg, 490 umol, 0.100 eq) under nitrogen atmosphere. The mixture was stirred at 110° C. for 6 h. The mixture was diluted with water (50.0 mL) and extracted with ethyl acetate (3×30.0 mL). The combined organic layer was washed with brine and dried over anhydrous sodium sulfate, filtered and concentrated to give crude product. The crude product was purified by silica gel chromatography to give 3-fluoro-4,5-dimethylaniline. ¹H NMR (400 MHz, DMSO-d₆) δ=6.22 (s, 1H), 6.14 (dd, J=1.9, 12.2 Hz, 1H), 5.01 (s, 2H), 2.10 (s, 3H), 1.94 (d, J=1.4 Hz, 3H). MS (ESI) m/z 140.2 [M+H]⁺

Step 3: To a solution of 3-fluoro-4,5-dimethylaniline (300 mg, 2.16 mmol, 1.00 eq) and pyridine (511 mg, 6.47 mmol, 521 uL, 3.00 eq) in Acetonitrile (5.00 mL) was added phenyl carbonochloridate (371 mg, 2.37 mmol, 297 uL, 1.10 eq) at 0° C. The mixture was stirred at 25° C. for 12 h. The mixture was concentrated to give crude product and purified by reversed-phase HPL to give phenyl (3-fluoro-4,5-dimethylphenyl)carbamate. MS (ESI) m/z 260.0 [M+H]⁺

Step 4: To a solution of 3-(6-(hydroxymethyl)-1-oxoisoindolin-2-yl)piperidine-2,6-dione VIII (80.0 mg, 292 umol, 1.00 eq) and phenyl (3-fluoro-4,5-dimethylphenyl)carbamate (83.1 mg, 320 umol, 1.10 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched with 1 M hydrochloric acid (1.00 mL) and purified by prep-HPLC. The desired fraction was collected and lyophilized to give (2-(2,6-dioxopiperidin-3-yl)-3-oxoisoindolin-5-yl)methyl(3-fluoro-4,5-dimethylphenyl)carbamate 117. ¹H NMR (400 MHz, DMSO-d₆) δ=10.99 (s, 1H), 9.85 (s, 1H), 7.79 (s, 1H), 7.71-7.66 (m, 1H), 7.66-7.61 (m, 1H), 7.19 (br d, J=11.7 Hz, 1H), 7.05 (s, 1H), 5.27 (s, 2H), 5.13 (dd, J=5.1, 13.3 Hz, 1H), 4.52-4.43 (m, 1H), 4.39-4.31 (m, 1H), 2.98-2.86 (m, 1H), 2.61 (br d, J=17.7 Hz, 1H), 2.41 (dt, J=8.8, 13.2 Hz, 1H), 2.21 (s, 3H), 2.06 (s, 3H), 2.04-1.98 (m, 1H). MS (ESI) m/z 440.1 [M+H]⁺

### Compound 318:

Step 1: To a solution of 2-methylpyridine-4-carbonitrile (1.00 g, 8.46 mmol, 1.00 eq), 2,2-dimethylpropanoic acid (951 mg, 9.31 mmol, 1.07 mL, 1.10 eq) and sulfuric acid (18.4 g, 18.8 mmol, 10.0 mL, 10% purity, 2.22 eq) in Acetonitrile (10.0 mL) was added silver nitrate (863 mg, 5.08 mmol, 0.600 eq). The mixture was stirred at 70° C. for 0.5 h, then ammonium persulfate (5.80 g, 25.4 mmol, 5.52 mL, 3.00 eq) in water (10.0 mL) was added to the mixture at 70° C. The mixture was stirred at 70° C. for 0.5 h. The reaction was cooled to 0° C. and 30% ammonium hydroxide was added slowly until pH=9-10. The mixture was filtered and extracted with ethyl acetate (3×100 mL). The combined organic phase was washed with brine, dried with anhydrous sodium sulfate, filtered and concentrated in vacuum. The residue was purified by column chromatography to afford 2-(tert-butyl)-6-methylisonicotinonitrile. ¹H NMR (400 MHz, CDCl₃) δ=7.34 (s, 1H), 7.17 (d, J=0.6 Hz, 1H), 2.59 (s, 3H), 1.36 (s, 9H).

Step 2: To a solution of 2-(tert-butyl)-6-methylisonicotinonitrile (950 mg, 5.45 mmol, 1.00 eq) and potassium carbonate (3.01 g, 21.8 mmol, 4.00 eq) in dimethyl sulfoxide (4.50 mL) and water (4.50 mL) was added hydrogen peroxide (5.31 g, 46.8 mmol, 4.50 mL, 30% purity, 8.59 eq) at 0° C. The mixture was stirred at 25° C. for 2 h. The reaction was quenched with sodium persulfate aqueous solution and extracted with ethyl acetate. The combined organic phase was washed with brine, dried over anhydrous sodium sulfate and then filtered. The filtration was concentrated in vacuum to afford 2-(tert-butyl)-6-methylisonicotinamide. ¹H NMR (400 MHz, CDCl₃) δ=7.47 (s, 1H), 7.25 (d, J=1.1 Hz, 1H), 6.34-5.99 (m, 2H), 2.59 (s, 3H), 1.37 (s, 9H).

Step 3: To a solution of 2-(tert-butyl)-6-methylisonicotinamide (1.00 g, 5.20 mmol, 1.00 eq) and N,N-diisopropylethylamine (739 mg, 5.72 mmol, 997 uL, 1.10 eq) in Acetonitrile (20.0 mL) and water (20.0 mL) was added (diacetoxyiodo)benzene (1.84 g, 5.72 mmol, 1.10 eq). The mixture was stirred at 30° C. for 3 h. The reaction mixture was concentrated under reduced pressure to remove Acetonitrile. The aqueous phase was added saturated sodium carbonate solution to pH=8 and extracted with ethyl acetate (3×20.0 mL). The combined organic layers were washed with brine (2×10.0 mL), dried over sodium sulfate, filtered and concentrated under reduced pressure. The residue was purified by Prep-HPLC and lyophilized to afford 2-(tert-butyl)-6-methylpyridin-4-amine. ¹H NMR (400 MHz, DMSO-d₆) δ=6.33 (d, J=1.6 Hz, 1H), 6.14 (d, J=1.5 Hz, 1H), 5.72 (br s, 2H), 2.21 (s, 3H), 1.21 (s, 9H).

Step 4: To a solution of 2-(tert-butyl)-6-methylpyridin-4-amine (0.100 g, 609 umol, 1.00 eq) and pyridine (241 mg, 3.04 mmol, 246 uL, 5.00 eq) in Acetonitrile (3.00 mL) was added phenyl carbonochloridate (143 mg, 913 umol, 114 uL, 1.50 eq) at 0° C. The mixture was stirred at 25° C. for 12 h. The mixture was concentrated in vacuum. The residue was purified by prep-HPLC and lyophilized to afford phenyl (2-(tert-butyl)-6-methylpyridin-4-yl)carbamate. ¹H NMR (400 MHz, DMSO-d₆) δ=10.49 (br s, 1H), 7.48-7.41 (m, 2H), 7.39 (s, 1H), 7.31-7.26 (m, 1H), 7.25-7.21 (m, 2H), 7.16 (s, 1H), 2.40 (s, 3H), 1.26 (s, 9H).

Step 5: To a solution of 3-(6-(hydroxymethyl)-1-oxoisoindolin-2-yl)piperidine-2,6-dione VIII (48.0 mg, 175 umol, 1.00 eq) and phenyl (2-(tert-butyl)-6-methylpyridin-4-yl)carbamate (59.7 mg, 210 umol, 1.20 eq) in dimethyl formamide (2.00 mL) was added sodium hydride (10.5 mg, 263 umol, 60% purity, 1.50 eq) at 0° C. The mixture was stirred at 0° C. for 1 h. The reaction was quenched by acetic acid (0.500 mL) slowly and then filtered and concentrated in vacuum. The residue was purified by prep-HPLC and lyophilized to afford (2-(2,6-dioxopiperidin-3-yl)-3-oxoisoindolin-5-yl)methyl(2-(tert-butyl)-6-methylpyridin-4-yl)carbamate 118. ¹H NMR (400 MHz, DMSO-d₆) δ=10.99 (s, 1H), 10.07 (s, 1H), 8.15 (s, 1H), 7.80 (s, 1H), 7.71-7.66 (m, 1H), 7.66-7.61 (m, 1H), 7.31 (d, J=1.3 Hz, 1H), 7.15 (d, J=1.5 Hz, 1H), 5.29 (s, 2H), 5.13 (dd, J=5.1, 13.3 Hz, 1H), 4.51-4.42 (m, 1H), 4.39-4.30 (m, 1H), 2.91 (ddd, J=5.4, 13.5, 17.5 Hz, 1H), 2.60 (br d, J=17.6 Hz, 1H), 2.46-2.39 (m, 1H), 2.37 (s, 3H), 2.07-1.97 (m, 1H), 1.25 (s, 9H). MS (ESI) m/z 465.2 [M+H]⁺

### Compound 319:

Step 1: To a solution of 1,4-dichloro-2-methylbenzene (5.00 g, 31.0 mmol, 1.00 eq) in sulfuric acid (7.00 mL) and acetic acid (3.00 mL) was added a mixture of nitric acid (3.08 g, 34.2 mmol, 2.20 mL, 70% purity, 1.10 eq) and sulfuric acid (4.05 g, 40.4 mmol, 2.20 mL, 98% purity, 1.30 eq) dropwise over 10 min. The mixture was warmed slowly to 10° C. and stirred for 2 h. The reaction was poured into ice water slowly and extracted with ethyl acetate. The combined organic layer was washed with brine and dried over anhydrous sodium sulfate, filtered and concentrated to give 1,4-dichloro-2-methyl-5-nitrobenzene. ¹H NMR (400 MHz, DMSO-d₆) δ=8.23 (s, 1H), 7.84 (s, 1H), 2.41 (s, 3H).

Step 2: A mixture of 1,4-dichloro-2-methyl-5-nitrobenzene (3.00 g, 14.6 mmol, 1.00 eq), iron powder (2.44 g, 43.7 mmol, 3.00 eq) and ammonium chloride (3.89 g, 72.8 mmol, 5.00 eq) in water (10.0 mL) and methanol (30.0 mL) was stirred at 80° C. for 2 h. The mixture was filtered and the filtrate was concentrated under reduced pressure. The residue was diluted with sodium hydrogencarbonate and extracted with ethyl acetate. The organic layer was dried over anhydrous sodium sulfate, filtered and concentrated in vacuum to give 2,5-dichloro-4-methylaniline. ¹H NMR (400 MHz, DMSO-d₆) δ=7.18 (s, 1H), 6.84 (s, 1H), 5.39 (br s, 2H), 2.15 (s, 3H). MS (ESI) m/z 176.1 [M+H]⁺

Step 3: To a solution of 2,5-dichloro-4-methylaniline (1.00 g, 5.68 mmol, 1.00 eq) and pyridine (1.35 g, 17.0 mmol, 1.38 mL, 3.00 eq) in Acetonitrile (10.0 mL) was added phenyl carbonochloridate (978 mg, 6.25 mmol, 783 uL, 1.10 eq) dropwise at 0° C. The mixture was stirred at 20° C. for 2 h. The mixture was concentrated in vacuum to give crude product. The crude product was purified by reversed-phase HPLC and lyophilized to give phenyl (2,5-dichloro-4-methylphenyl)carbamate. MS (ESI) m/z 296.0 [M+H]⁺

Step 4: To a solution of 3-(6-(hydroxymethyl)-1-oxoisoindolin-2-yl)piperidine-2,6-dione VIII (80.0 mg, 292 umol, 1.00 eq) and phenyl (2,5-dichloro-4-methylphenyl)carbamate (95.0 mg, 321 umol, 1.10 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched with 1 M hydrochloric acid (1.00 mL), purified by prep-HPLC and the desired fraction was lyophilized to give (2-(2,6-dioxopiperidin-3-yl)-3-oxoisoindolin-5-yl)methyl(2,5-dichloro-4-methylphenyl)carbamate 119. ¹H NMR (400 MHz, DMSO-d₆) δ=11.00 (s, 1H), 9.38 (s, 1H), 7.82 (s, 1H), 7.73-7.59 (m, 3H), 7.53 (s, 1H), 5.28 (s, 2H), 5.14 (dd, J=5.1, 13.3 Hz, 1H), 4.53-4.44 (m, 1H), 4.39-4.31 (m, 1H), 2.99-2.86 (m, 1H), 2.61 (br d, J=17.4 Hz, 1H), 2.41 (dt, J=8.9, 13.2 Hz, 1H), 2.30 (s, 3H), 2.06-1.98 (m, 1H). MS (ESI) m/z 476.1 [M+H]⁺

### Compound 320:

To a solution of 3-(tert-butyl)bicyclo[1.1.1]pentane-1-carboxylic acid (25.0 mg, 148 umol, 1.00 eq) in dioxane (2.00 mL) was added triethylamine (45.4 mg, 449 umol, 62.5 uL, 3.02 eq) and (79.3 mg, 288 umol, 62.5 uL, 1.94 eq) at 20° C. The mixture was stirred at 20° C. for 1 h. Then 3-(6-(hydroxymethyl)-1-oxoisoindolin-2-yl)piperidine-2,6-dione VIII (45.0 mg, 164 umol, 1.10 eq) was added. The mixture was stirred at 100° C. for 2 h under nitrogen atmosphere. The mixture was concentrated under reduced pressure. The residue was purified by Prep-HPLC and the desired fraction was collected and lyophilized to give (2-(2,6-dioxopiperidin-3-yl)-3-oxoisoindolin-5-yl)methyl (3-(tert-butyl) bicyclo[1.1.1]pentan-1-yl)carbamate 120. ¹H NMR (400 MHz, DMSO-d₆) δ=10.98 (br s, 1H), 7.93 (br s, 1H), 7.71 (s, 1H), 7.61 (s, 2H), 5.23-5.02 (m, 3H), 4.51-4.41 (m, 1H), 4.38-4.29 (m, 1H), 2.98-2.86 (m, 1H), 2.65-2.58 (m, 1H), 2.41 (br dd, J=4.4, 13.2 Hz, 1H), 2.06-1.97 (m, 1H), 1.71 (s, 6H), 0.84 (s, 9H). MS (ESI) m/z 440.2 [M+H]⁺

### Compound 321:

Step 1: To a solution of 2-(2,6-dioxo-1-((2-(trimethylsilyl)ethoxy)methyl)piperidin-3-yl)-3-oxoisoindoline-5-carbaldehyde (1.00 g, 2.48 mmol, 1.00 eq) in dichloromethane (10.0 mL) at −78° C. was added methyl magnesium bromide (3.00 M in diethyl ether, 1.24 mL, 1.50 eq) drop-wise over 5 min under nitrogen atmosphere. Then the mixture was stirred at 0° C. for 3 h. The reaction was quenched with saturated ammonium chloride aqueous solution (10.0 mL). The reaction mixture was diluted with water (100 mL) and extracted with ethyl acetate (100 mL). The organic layer was washed with brine (50 mL), dried over anhydrous sodium sulfate, filtered and concentrated under reduced pressure to give a residue. The residue was purified by column chromatography to afford 3-(6-(1-hydroxyethyl)-1-oxoisoindolin-2-yl)-1-((2-(trimethylsilyl)ethoxy)methyl)piperidine-2,6-dione. ¹H NMR (400 MHz, DMSO-d₆) δ=7.71 (s, 1H), 7.64-7.58 (m, 1H), 7.57-7.50 (m, 1H), 5.31 (d, J=4.5 Hz, 1H), 5.26-5.20 (m, 1H), 5.10-5.00 (m, 2H), 4.88-4.81 (m, 1H), 4.45 (br d, J=17.1 Hz, 1H), 4.31-4.22 (m, 1H), 3.59-3.47 (m, 2H), 3.13-2.99 (m, 1H), 2.85-2.73 (m, 1H), 2.46-2.31 (m, 1H), 1.35 (d, J=6.5 Hz, 3H), 0.87-0.81 (m, 2H), −0.01-−0.07 (m, 9H).

Step 2: A mixture of 3-(6-(1-hydroxyethyl)-1-oxoisoindolin-2-yl)-1-((2-(trimethylsilyl)ethoxy)methyl)piperidine-2,6-dione (700 mg, 1.67 mmol, 1.00 eq) in hydrochloric acid/dioxane (4.00 M, 20.0 mL, 47.8 eq) was stirred at 25° C. for 3 h. The mixture was concentrated under reduced pressure to afford 3-(6-(1-hydroxyethyl)-1-oxoisoindolin-2-yl)-1-(hydroxymethyl)piperidine-2,6-dione.

Step 3: A mixture of 3-(6-(1-hydroxyethyl)-1-oxoisoindolin-2-yl)piperidine-2,6-dione (500 mg, 1.57 mmol, 1.00 eq) and ammonium hydroxide (91.0 mg, 649 umol, 0.100 mL, 25% purity) in Acetonitrile (5.00 mL) was stirred at 25° C. for 5 h. The pH of the mixture was adjusted to 3-4 with hydrochloric acid (1 M). The mixture was concentrated under reduced pressure to give a residue, which was purified by reversed phase column chromatography and lyophilized to afford 3-(6-(1-hydroxyethyl)-1-oxoisoindolin-2-yl) piperidine-2,6-dione. ¹H NMR (400 MHz, DMSO-d₆) δ=10.98 (s, 1H), 7.75 (br s, 1H), 7.64-7.58 (m, 1H), 7.57-7.50 (m, 1H), 5.30 (br d, J=3.5 Hz, 1H), 5.15-4.99 (m, 1H), 4.84 (br d, J=5.1 Hz, 1H), 4.50-4.38 (m, 1H), 4.34-4.23 (m, 1H), 3.12-2.85 (m, 1H), 2.60 (br d, J=17.4 Hz, 1H), 2.47-2.30 (m, 1H), 2.01 (br d, J=6.1 Hz, 1H), 1.35 (br d, J=6.4 Hz, 3H).

Step 4: A mixture of 3-(trifluoromethoxy) aniline (500 mg, 2.82 mmol, 376 uL, 1.00 eq), pyridine (670 mg, 8.47 mmol, 684 uL, 3.00 eq) and phenyl carbonochloridate (663 mg, 4.23 mmol, 530 uL, 1.50 eq) in Acetonitrile (5.00 mL) was stirred at 25° C. for 12 h. The mixture was concentrated under reduced pressure to give a residue. The residue was purified by reversed phase column chromatography (C18; condition: water/Acetonitrile=1/0 to 0/1, 0.1% Formic acid) and lyophilized to afford phenyl (3-(trifluoromethoxy)phenyl)carbamate (680 mg, 2.29 mmol, 81% yield) as colorless oil. MS (ESI) m/z 298.1 [M+H]⁺

Step 5: To a mixture of 3-(6-(1-hydroxyethyl)-1-oxoisoindolin-2-yl)piperidine-2,6-dione (85.0 mg, 295 umol, 1.00 eq) and phenyl (3-(trifluoromethoxy)phenyl)carbamate (105 mg, 354 umol, 1.20 eq) in dimethyl formamide (2.00 mL) was added sodium hydride (23.6 mg, 590 umol, 60% purity, 2.00 eq) under nitrogen atmosphere and the mixture was stirred at 0° C. for 3 h. The mixture was quenched with hydrochloric acid (1 M) and filtered. The filtrate was purified by Prep-HPLC and lyophilized to afford 1-(2-(2,6-dioxopiperidin-3-yl)-3-oxoisoindolin-5-yl)ethyl(3-(trifluoromethoxy)phenyl)carbamate 121. ¹H NMR (400 MHz, DMSO-d₆) δ=10.99 (s, 1H), 10.14 (s, 1H), 8.47 (s, 1H), 7.78 (s, 1H), 7.70-7.65 (m, 1H), 7.65-7.60 (m, 1H), 7.56 (s, 1H), 7.42-7.37 (m, 2H), 6.96 (br d, J=3.8 Hz, 1H), 5.97-5.89 (m, 1H), 5.16-5.09 (m, 1H), 4.50-4.42 (m, 1H), 4.37-4.29 (m, 1H), 2.97-2.85 (m, 1H), 2.65-2.56 (m, 1H), 2.46-2.34 (m, 1H), 2.01-1.99 (m, 1H), 1.58 (d, J=6.8 Hz, 3H). MS (ESI) m/z 492.1 [M+H]⁺

### Compound 322:

Step 1: To a solution of 2,4-dimethyl-6-nitroaniline (5.00 g, 30.1 mmol, 1.00 eq) in tetrahydrofuran (50.0 mL) was added nitrosonium tetrafluoroborate (5.27 g, 45.1 mmol, 1.50 eq) in portions at −5° C. After the mixture was stirred at −5° C. for 0.5 h, the mixture was filtered. The filter cake was washed with tetrahydrofuran (20.0 mL) then dissolved in 1,2-dichlorobenzene (50.0 mL) and heated at 170° C. for 1 h. The mixture was filtered and the filtrate was concentrated to give crude product. The crude product was purified by silica gel chromatography to give 2-fluoro-1,5-dimethyl-3-nitrobenzene. ¹H NMR (400 MHz, DMSO-d₆) δ=7.79-7.74 (m, 1H), 7.55-7.50 (m, 1H), 2.33 (s, 3H), 2.29 (d, J=2.3 Hz, 3H).

Step 2: To a mixture of 2-fluoro-1,5-dimethyl-3-nitro-benzene (1.00 g, 5.91 mmol, 1.00 eq) in tetrahydrofuran (10.0 mL) was added Pd/C (200 mg, 10% purity) under nitrogen. The suspension was degassed under vacuum and purged with hydrogen several times. The mixture was stirred at 20° C. for 2 h under hydrogen (15 psi). The mixture was filtered and the filtrate was concentrated to give crude product then purified by reversed-phase HPLC to give 2-fluoro-3,5-dimethylaniline. ¹H NMR (400 MHz, DMSO-d₆) δ=6.38 (dd, J=1.8, 8.3 Hz, 1H), 6.25-6.10 (m, 1H), 4.88 (s, 2H), 2.10 (s, 6H). MS (ESI) m/z 140.1 [M+H]

Step 3: To a solution of 2-fluoro-3,5-dimethylaniline (150 mg, 1.08 mmol, 1.00 eq) and pyridine (426 mg, 5.39 mmol, 435 uL, 5.00 eq) in Acetonitrile (2.00 mL) was added phenyl carbonochloridate (202 mg, 1.29 mmol, 162 uL, 1.20 eq) dropwise at 0° C. The mixture was stirred at 25° C. for 2 h. The mixture was concentrated to give crude product then purified reversed-phase HPLC to give phenyl (2-fluoro-3,5-dimethylphenyl). MS (ESI) m/z 260.1 [M+H]

Step 4: To a solution of 3-(6-(hydroxymethyl)-1-oxoisoindolin-2-yl)piperidine-2,6-dione VIII (80.0 mg, 292 umol, 1.00 eq) and phenyl (2-fluoro-3,5-dimethylphenyl)carbamate (83.2 mg, 321 umol, 1.10 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched by 1 M hydrochloric acid (1 mL) then filtered, the filtrate was purified by prep-HPLC and lyophilized to give (2-(2,6-dioxopiperidin-3-yl)-3-oxoisoindolin-5-yl)methyl (2-fluoro-3,5-dimethylphenyl)carbamate 122. ¹H NMR (400 MHz, DMSO-d₆) δ=11.00 (s, 1H), 9.38 (br s, 1H), 7.80 (s, 1H), 7.72-7.59 (m, 2H), 7.28 (br d, J=6.1 Hz, 1H), 6.82 (br d, J=5.6 Hz, 1H), 5.26 (s, 2H), 5.14 (dd, J=5.0, 13.3 Hz, 1H), 4.55-4.42 (m, 1H), 4.39-4.27 (m, 1H), 2.99-2.85 (m, 1H), 2.61 (br d, J=18.1 Hz, 1H), 2.41 (dt, J=8.8, 13.1 Hz, 1H), 2.22 (s, 3H), 2.19 (d, J=1.8 Hz, 3H), 2.06-1.98 (m, 1H). MS (ESI) m/z 440.1 [M+H]⁺

### Compound 323:

Step 1: A mixture of 1-chloro-2,4-dimethyl-5-nitrobenzene (1.00 g, 5.39 mmol, 1.00 eq), iron powder (903 mg, 16.2 mmol, 3.00 eq) and ammonium chloride (1.44 g, 26.9 mmol, 5.00 eq) in water (3.00 mL) and methanol (10.0 mL) was stirred at 80° C. for 2 h. The mixture was filtered and the filtrate was concentrated under reduced pressure. The mixture was diluted with sodium hydrogen carbonate and extracted with ethyl acetate. The organic layer was dried over sodium sulfate, filtered and concentrated in vacuum to give 5-chloro-2,4-dimethylaniline. ¹H NMR (400 MHz, DMSO-d₆) δ=6.85 (s, 1H), 6.64 (s, 1H), 4.89 (br s, 2H), 2.12 (s, 3H), 1.99 (s, 3H). MS (ESI) m/z 156.2 [M+H]

Step 2: To a solution of 5-chloro-2,4-dimethylaniline (400 mg, 2.57 mmol, 1.00 eq) and pyridine (610 mg, 7.71 mmol, 622 uL, 3.00 eq) in Acetonitrile (5.00 mL) was added phenyl carbonochloridate (604 mg, 3.86 mmol, 483 uL, 1.50 eq) dropwise at 0° C. The mixture was then stirred at 25° C. for 12 h. The mixture was concentrated in vacuum to give crude product. The crude product was purified by reversed-phase HPLC and lyophilized to give phenyl (5-chloro-2,4-dimethylphenyl)carbamate. MS (ESI) m/z 276.1 [M+H]

Step 3: To a solution of 3-(6-(hydroxymethyl)-1-oxoisoindolin-2-yl)piperidine-2,6-dione VIII (80.0 mg, 292 umol, 1.00 eq) and phenyl (5-chloro-2,4-dimethylphenyl)carbamate (88.5 mg, 321 umol, 1.10 eq) in dimethyl formamide (1.00 mL) was added sodium hydride (23.3 mg, 583 umol, 60% purity, 2.00 eq) at 0° C. The mixture was stirred at 20° C. for 1 h. The mixture was quenched by 1 M hydrochloric acid (1.00 mL) then purified by prep-HPLC and lyophilized to give (2-(2,6-dioxopiperidin-3-yl)-3-oxoisoindolin-5-yl) methyl (5-chloro-2,4-dimethylphenyl)carbamate 123. ¹H NMR (400 MHz, DMSO-d₆) δ=11.00 (s, 1H), 9.11 (s, 1H), 7.80 (s, 1H), 7.72-7.60 (m, 2H), 7.46 (s, 1H), 7.17 (s, 1H), 5.27 (s, 2H), 5.13 (dd, J=5.1, 13.4 Hz, 1H), 4.53-4.43 (m, 1H), 4.41-4.29 (m, 1H), 3.00-2.84 (m, 1H), 2.61 (br d, J=16.8 Hz, 1H), 2.46-2.36 (m, 1H), 2.26 (s, 3H), 2.16 (s, 3H), 2.06-1.97 (m, 1H). MS (ESI) m/z 456.1 [M+H]⁺

### Compound 324:

Step 1: To a mixture of 4-fluoro-3,5-dimethylaniline (500 mg, 3.59 mmol, 1.00 eq) and pyridine (853 mg, 10.8 mmol, 870 uL, 3.00 eq) in Acetonitrile (5.00 mL) was added phenyl carbonochloridate (731 mg, 4.67 mmol, 585 uL, 1.30 eq) dropwise. The mixture was stirred at 15° C. for 12 h. The mixture was concentrated to give crude product. The crude product was purified by reversed-phase column and the desired fraction was collected and lyophilized to give phenyl (4-fluoro-3,5-dimethylphenyl)carbamate. 1H NMR (400 MHz, DMSO-d₆) δ=10.09 (br s, 1H), 7.48-7.43 (m, 2H), 7.41-7.36 (m, 3H), 7.20 (br d, J=7.6 Hz, 2H), 2.18 (d, J=2.0 Hz, 6H).

Step 2: To a mixture of phenyl (4-fluoro-3,5-dimethylphenyl)carbamate (113 mg, 438 umol, 1.20 eq) and 3-(6-(hydroxymethyl)-1-oxoisoindolin-2-yl)piperidine-2,6-dione VIII (100 mg, 365 umol, 1.00 eq) in dimethyl formamide (2.00 mL) was added sodium hydride (21.9 mg, 547 umol, 60% purity, 1.50 eq) in portions at 0° C. The mixture was stirred at 15° C. for 1 h. The mixture was quenched with 1 M hydrochloric and filtered. The filtrate was purified by prep-HPLC and lyophilized to give (2-(2,6-dioxopiperidin-3-yl)-3-oxoisoindolin-5-yl)methyl (4-fluoro-3,5-dimethylphenyl)carbamate 124. ¹H NMR (400 MHz, DMSO-d₆) δ=10.99 (br s, 1H), 9.68 (br s, 1H), 7.79 (s, 1H), 7.70-7.62 (m, 2H), 7.17 (br d, J=6.4 Hz, 2H), 5.26 (s, 2H), 5.13 (dd, J=5.2, 13.2 Hz, 1H), 4.53-4.43 (m, 1H), 4.40-4.30 (m, 1H), 3.00-2.85 (m, 1H), 2.65-2.58 (m, 1H), 2.46-2.36 (m, 1H), 2.17 (d, J=1.6 Hz, 6H), 2.06-1.98 (m, 1H). MS (ESI) m/z 440.1 [M+H]⁺

### Compound 325:

Step 1: To a mixture of 2-chloro-5-(trifluoromethoxy)aniline (500 mg, 2.36 mmol, 1.00 eq) and pyridine (561 mg, 7.09 mmol, 572 uL, 3.00 eq) in Acetonitrile (5.00 mL) was added phenyl carbonochloridate (481 mg, 3.07 mmol, 385 uL, 1.30 eq) dropwise. The mixture was stirred at 15° C. for 2 h. The mixture was concentrated to give crude product. The crude product was purified by reversed-phase column and the desired fraction was collected and lyophilized to give phenyl (2-chloro-5-(trifluoromethoxy)phenyl)carbamate. ¹H NMR (400 MHz, DMSO-d₆) δ=9.95 (s, 1H), 7.78 (d, J=2.3 Hz, 1H), 7.66 (d, J=8.9 Hz, 1H), 7.50-7.39 (m, 2H), 7.33-7.19 (m, 4H).

Step 2: To a mixture of phenyl (2-chloro-5-(trifluoromethoxy)phenyl)carbamate (116 mg, 350 umol, 1.20 eq) and 3-(6-(hydroxymethyl)-1-oxoisoindolin-2-yl)piperidine-2,6-dione VIII (80.0 mg, 292 umol, 1.00 eq) in dimethyl formamide (2.00 mL) was added sodium hydride (17.5 mg, 438 umol, 60% purity, 1.50 eq) in one portion at 0° C. The mixture was stirred at 15° C. for 1 h. The mixture was quenched with 1 M hydrochloric and filtered. The filtrate was purified by prep-HPLC and lyophilized to give (2-(2,6-dioxopiperidin-3-yl)-3-oxoisoindolin-5-yl)methyl(2-chloro-5-(trifluoromethoxy)phenyl)carbamate 125. ¹H NMR (400 MHz, DMSO-d₆) δ=10.99 (s, 1H), 9.53 (s, 1H), 7.83 (s, 1H), 7.75 (d, J=2.2 Hz, 1H), 7.72-7.66 (m, 1H), 7.66-7.59 (m, 2H), 7.20 (dd, J=2.4, 8.6 Hz, 1H), 5.31 (s, 2H), 5.13 (dd, J=5.1, 13.3 Hz, 1H), 4.53-4.42 (m, 1H), 4.40-4.30 (m, 1H), 2.97-2.86 (m, 1H), 2.60 (br d, J=17.9 Hz, 1H), 2.46-2.35 (m, 1H), 2.06-1.98 (m, 1H). MS (ESI) m/z 512.1 [M+H]⁺

### Compound 326:

To a solution of 3-phenylbicyclo[1.1.1]pentane-1-carboxylic acid (10.0 mg, 53.1 umol, 1.00 eq) in dioxane (2.00 mL) was added triethylamine (18.1 mg, 179 umol, 25.0 uL, 3.38 eq) and diphenylphosphoryl azide (25.4 mg, 92.3 umol, 20.0 uL, 1.74 eq) at 20° C. The mixture was stirred at 20° C. for 1 h. Then 3-(6-(hydroxymethyl)-1-oxoisoindolin-2-yl)piperidine-2,6-dione VIII (15.0 mg, 54.6 umol, 1.03 eq) was added. The mixture was stirred at 100° C. for 2 h. The mixture was concentrated under reduced pressure. The residue was purified by Prep-HPLC and the desired fraction was collected and lyophilized to give (2-(2,6-dioxopiperidin-3-yl)-3-oxoisoindolin-5-yl)methyl (3-phenylbicyclo[1.1.1]pentan-1-yl carbamate 126. ¹H NMR (400 MHz, DMSO-d₆) δ=10.89 (brs, 1H), 8.11 (brs, 1H), 7.73 (s, 1H), 7.63 (s, 2H), 7.34-7.27 (m, 2H), 7.23 (br d, J=6.5 Hz, 3H), 5.22-5.07 (m, 3H), 4.50-4.42 (m, 1H), 4.39-4.30 (m, 1H), 2.98-2.86 (m, 1H), 2.64-2.59 (m, 1H), 2.41 (br dd, J=4.3, 13.1 Hz, 1H), 2.20 (s, 6H), 2.06-1.98 (m, 1H). MS (ESI) m/z 460.2 [M+H]⁺

### Compound 327:

Step 2: To a solution of 1,3-difluoro-2-methyl-5-nitrobenzene (500 mg, 2.89 mmol, 1.00 eq) in tetrahydrofuran (5.00 mL) was added palladium on carbon (80.0 mg, 10% purity). The mixture was stirred under hydrogen at 25° C. for 3 h. The mixture was filtered, and the filtrate was concentrated under reduced pressure to afford 3,5-difluoro-4-methylaniline. ¹H NMR (400 MHz, DMSO-d₆) δ=6.16 (d, J=10.0 Hz, 2H), 5.45 (s, 2H), 2.55-2.46 (m, 3H).

Step 2: To a solution of 3,5-difluoro-4-methylaniline (180 mg, 1.26 mmol, 1.00 eq) in Acetonitrile (3.00 mL) was added pyridine (298 mg, 3.77 mmol, 304 uL, 3.00 eq) and phenyl carbonochloridate (236 mg, 1.51 mmol, 189 uL, 1.20 eq). The mixture was stirred at 25° C. for 3 h. The reaction mixture was concentrated under reduced pressure to afford a residue. The residue was diluted with water (40.0 mL) and extracted with ethyl acetate (3×20.0 mL). The organic layer was washed with brine, dried over anhydrous sodium sulfate, filtered and concentrated under reduced pressure to afford a residue. The residue was purified by Reversed-Phase Flash and the desired eluent was lyophilized to afford phenyl (3,5-difluoro-4-methylphenyl)carbamate. ¹H NMR (400 MHz, DMSO-d₆) δ=10.54 (br s, 1H), 7.51-7.38 (m, 2H), 7.33-7.10 (m, 5H), 2.09 (s, 3H). MS (ESI) m/z 264.1 [M+H]⁺

Step 3: To a solution of phenyl (3,5-difluoro-4-methylphenyl) carbamate (107 mg, 408 umol, 1.40 eq) and 3-(6-(hydroxymethyl)-1-oxoisoindolin-2-yl)piperidine-2,6-dione VIII (80.0 mg, 291 umol, 1.00 eq) in dimethyl formamide (1.00 mL) was added sodium hydride (23.3 mg, 583 umol, 60% purity, 2.00 eq). The mixture was stirred at 25° C. for 1 h. The pH of the mixture was adjusted to 7 with 1 M hydrochloric acid, then the mixture was diluted with dimethyl formamide (1.00 mL). The mixture was purified by Prep-HPLC and lyophilized in vacuo to afford (2-(2,6-dioxopiperidin-3-yl)-3-oxoisoindolin-5-yl)methyl (3,5-difluoro-4-methylphenyl)carbamate 127. ¹H NMR (400 MHz, DMSO-d₆) δ=11.00 (s, 1H), 10.14 (s, 1H), 7.79 (s, 1H), 7.73-7.58 (m, 2H), 7.26-7.06 (m, 2H), 5.28 (s, 2H), 5.13 (dd, J=5.2, 13.2 Hz, 1H), 4.51-4.42 (m, 1H), 4.39-4.30 (m, 1H), 2.97-2.85 (m, 1H), 2.63-2.56 (m, 1H), 2.40 (br dd, J=4.4, 13.2 Hz, 1H), 2.09-2.04 (m, 3H), 2.04-1.97 (m, 1H). MS (ESI) m/z 444.2 [M+H]⁺

### Compound 328:

Step 1: To a mixture of 2-methyl-5-(trifluoromethoxy)aniline (500 mg, 2.62 mmol, 1.00 eq) and pyridine (621 mg, 7.85 mmol, 633 uL, 3.00 eq) in Acetonitrile (5.00 mL) was added phenyl carbonochloridate (614 mg, 3.92 mmol, 491 uL, 1.50 eq) dropwise at 0° C. The mixture was stirred at 25° C. for 12 h. The mixture was concentrated in vacuum to give crude product. The crude product was purified by reversed-phase HPLC and lyophilized to give phenyl (2-methyl-5-(trifluoromethoxy)phenyl)carbamate. MS (ESI) m/z 312.0 [M+H]⁺

Step 2: To a solution of 3-(6-(hydroxymethyl)-1-oxoisoindolin-2-yl)piperidine-2,6-dione VIII (100 mg, 365 umol, 1.00 eq) and phenyl (2-methyl-5-(trifluoromethoxy)phenyl)carbamate (136 mg, 438 umol, 1.20 eq) in dimethyl formamide (1.00 mL) was added sodium hydride (29.2 mg, 729 umol, 60% purity, 2.00 eq) at 0° C. The mixture was stirred at 20° C. for 1 h. The mixture was quenched by 1 M hydrochloric acid (2.00 mL) and filtered. The filtrate was purified by prep-HPLC and lyophilized to give (2-(2,6-dioxopiperidin-3-yl)-3-oxoisoindolin-5-yl)methyl(2-methyl-5-(trifluoromethoxy)phenyl)carbamate 128. ¹H NMR (400 MHz, DMSO-d₆) δ=11.00 (s, 1H), 9.24 (s, 1H), 7.82 (s, 1H), 7.72-7.68 (m, 1H), 7.67-7.62 (m, 1H), 7.52 (br s, 1H), 7.31 (d, J=8.4 Hz, 1H), 7.04 (dd, J=1.3, 8.3 Hz, 1H), 5.29 (s, 2H), 5.14 (dd, J=5.1, 13.4 Hz, 1H), 4.53-4.44 (m, 1H), 4.39-4.31 (m, 1H), 2.98-2.86 (m, 1H), 2.61 (br d, J=17.6 Hz, 1H), 2.47-2.36 (m, 1H), 2.24 (s, 3H), 2.07-1.97 (m, 1H). MS (ESI) m/z 492.1 [M+H]⁺

### Compound 329:

Step 1: To a mixture of 3,4-dimethylaniline (500 mg, 4.13 mmol, 1.00 eq) and pyridine (979 mg, 12.4 mmol, 999 uL, 3.00 eq) in Acetonitrile (5.00 mL) was added phenyl carbonochloridate (969 mg, 6.19 mmol, 775 uL, 1.50 eq) dropwise. The mixture was stirred at 15° C. for 2 h. The mixture was concentrated to give crude product. The crude product was purified by reversed-phase HPLC. The desired fraction was collected and lyophilized to give phenyl (3,4-dimethylphenyl)carbamate. ¹H NMR (400 MHz, DMSO-d₆) δ=10.05 (br s, 1H), 7.44-7.40 (m, 2H), 7.31-7.19 (m, 5H), 7.07 (d, J=8.1 Hz, 1H), 2.18 (d, J=8.7 Hz, 6H).

Step 2: To a mixture of phenyl (3,4-dimethylphenyl)carbamate (84.5 mg, 350 umol, 1.20 eq) and 3-(6-(hydroxymethyl)-1-oxoisoindolin-2-yl)piperidine-2,6-dione VIII (80.0 mg, 292 umol, 1.00 eq) in dimethyl formamide (2.00 mL) was added sodium hydride (17.5 mg, 438 umol, 60% purity, 1.50 eq) in portions at 0° C. The mixture was stirred at 15° C. for 2 h. The mixture was quenched with 1 M hydrochloric and filtered. The filtrate was purified by prep-HPLC and lyophilized to give (2-(2,6-dioxopiperidin-3-yl)-3-oxoisoindolin-5-yl)methyl(3,4-dimethylphenyl)carbamate 129. ¹H NMR (400 MHz, DMSO-d₆) δ=10.99 (s, 1H), 9.61 (br s, 1H), 7.78 (s, 1H), 7.70-7.60 (m, 2H), 7.24 (s, 1H), 7.18 (br d, J=8.3 Hz, 1H), 7.02 (d, J=8.2 Hz, 1H), 5.25 (s, 2H), 5.12 (dd, J=5.0, 13.2 Hz, 1H), 4.51-4.43 (m, 1H), 4.38-4.30 (m, 1H), 2.97-2.86 (m, 1H), 2.60 (br d, J=17.6 Hz, 1H), 2.44-2.34 (m, 1H), 2.15 (d, J=9.0 Hz, 6H), 2.06-1.96 (m, 1H). MS (ESI) m/z 422.1 [M+H]⁺

### Compound 330:

Step 1: To a solution of 2-aminopropane-1,3-diol (10.0 g, 110 mmol, 1.00 eq) in ethyl alcohol (90.0 mL) was added triethylamine (16.7 g, 165 mmol, 23.0 mL, 1.51 eq) and benzyl carbonochloridate (56.2 g, 329 mmol, 46.8 mL, 3.00 eq) at 0° C. The mixture was stirred at 25° C. for 1 h. The mixture was filtered to give filtrate solution that was concentrated to give a residue. The residue was purified by column chromatography on silica gel. The desired fraction was collected and concentrated to give benzyl (1,3-dihydroxypropan-2-yl)carbamate. ¹H NMR (400 MHz, DMSO-d₆) δ=7.41-7.28 (m, 5H), 6.87 (br d, J=7.8 Hz, 1H), 5.01 (s, 2H), 4.58 (t, J=5.4 Hz, 2H), 3.50-3.36 (m, 5H).

Step 2: To a solution of benzyl (1,3-dihydroxypropan-2-yl)carbamate (3.00 g, 13.3 mmol, 1.00 eq) in toluene (40.0 mL) was added benzaldehyde (2.83 g, 26.6 mmol, 2.69 mL, 2.00 eq) and p-toluenesulfonic acid (230 mg, 1.34 mmol, 0.100 eq). The mixture was refluxed under a Dean-Stark water separator for 2 h at 130° C. The mixture was concentrated to give a residue, which was purified by column chromatography on silica gel. The desired fraction was collected and concentrated to give benzyl (2-phenyl-1,3-dioxan-5-yl)carbamate. ¹H NMR (400 MHz, CDCl₃) δ=7.51-7.34 (m, 10H), 5.86 (br d, J=8.4 Hz, 1H), 5.56 (s, 1H), 5.14 (s, 2H), 4.20-4.14 (m, 4H), 3.77 (br d, J=9.0 Hz, 1H).

Step 3: To a solution of benzyl (2-phenyl-1,3-dioxan-5-yl)carbamate (1.28 g, 4.08 mmol, 1.00 eq) in ethyl alcohol (10.0 mL) was added wet palladium on carbon (30.0 mg, 10% purity) under nitrogen. The mixture was stirred at 25° C. for 12 h under hydrogen (15 psi). The mixture was filtered to give filtrate that was concentrated to give 2-phenyl-1,3-dioxan-5-amine. ¹H NMR (400 MHz, CDCl₃) δ=7.53-7.36 (m, 5H), 5.54 (s, 1H), 4.21-4.15 (m, 2H), 4.14-3.99 (m, 2H), 2.89-2.78 (m, 1H), 1.79 (br s, 2H). MS (ESI) m/z 180.3 [M+H]⁺.

Step 4: To a solution of 2-phenyl-1,3-dioxan-5-amine (350 mg, 1.95 mmol, 1.00 eq) in Acetonitrile (10.0 mL) was added pyridine (463 mg, 5.86 mmol, 473 uL, 3.00 eq) and phenyl carbonochloridate (459 mg, 2.93 mmol, 367 uL, 1.50 eq) at 0° C. The mixture was stirred at 0° C. for 1 h. The mixture was filtered to give filtrate. The filtrate was purified by reversed-phase HPLC (0.1% Formic acid). The desired fraction was collected and concentrated to give phenyl (2-phenyl-1,3-dioxan-5-yl)carbamate. ¹H NMR (400 MHz, DMSO-d₆) δ=8.24 (br d, J=6.5 Hz, 1H), 7.58-7.50 (m, 2H), 7.41-7.35 (m, 5H), 7.23-7.18 (m, 1H), 7.13 (br d, J=7.6 Hz, 2H), 5.60 (s, 1H), 4.12 (q, J=11.5 Hz, 4H), 3.37 (s, 1H). MS (ESI) m/z 300.1 [M+H]⁺.

Step 5: To a solution of 3-(6-(hydroxymethyl)-1-oxoisoindolin-2-yl)piperidine-2,6-dione VIII (80.0 mg, 292 umol, 1.00 eq) in dimethyl formamide (2.00 mL) was added phenyl (2-phenyl-1,3-dioxan-5-yl)carbamate (131 mg, 438 umol, 1.50 eq) and sodium hydride (23.3 mg, 583 umol, 60% purity, 2.00 eq) at 0° C. The mixture was stirred at 0° C. for 1 h. The mixture was added hydrochloric acid (0.500 ml, 1 M). The mixture was extracted with water/ethyl acetate (10.0 ml/10.0 ml). The organic layer was collected and concentrated to give a residue. The residue was purified by prep-HPLC and the desired fraction was collected and lyophilized to give (2-(2,6-dioxopiperidin-3-yl)-3-oxoisoindolin-5-yl)methyl(2-phenyl-1,3-dioxan-5-yl)carbamate 128. ¹H NMR (400 MHz, DMSO-d₆) δ=10.99 (s, 1H), 7.86-7.73 (m, 2H), 7.67-7.59 (m, 2H), 7.54-7.46 (m, 2H), 7.43-7.31 (m, 3H), 5.57 (s, 1H), 5.18 (s, 2H), 5.12 (br dd, J=5.1, 13.3 Hz, 1H), 4.51-4.42 (m, 1H), 4.37-4.27 (m, 1H), 4.16-4.07 (m, 2H), 4.06-3.98 (m, 2H), 3.50 (br d, J=6.1 Hz, 1H), 3.04-2.76 (m, 1H), 2.60 (br d, J=16.8 Hz, 1H), 2.40 (br d, J=8.4 Hz, 1H), 2.04-1.97 (m, 1H). MS (ESI) m/z 480.2 [M+H]⁺.

### Compound 331:

Step 1: To a solution of 5-tert-butyl-2-chloro-pyridine (770 mg, 4.54 mmol, 1.00 eq), diphenylmethanimine (987 mg, 5.45 mmol, 914 uL, 1.20 eq), cesium carbonate (2.22 g, 6.81 mmol, 1.50 eq) and 4,5-bis(diphenylphosphino)-9,9-dimethylxanthene (525 mg, 908 umol, 0.200 eq) in dioxane (25.0 mL) was added tris(dibenzylideneacetone)dipalladium (416 mg, 454 umol, 0.100 eq). The mixture was stirred at 90° C. for 2 h. The mixture was filtered and concentrated in vacuum. The crude product was purified by reversed-phase HPLC to afford 5-(tert-butyl)-N-(diphenylmethylene)pyridin-2-amine. ¹H NMR (400 MHz, DMSO-d₆) δ=8.26 (d, J=2.0 Hz, 1H), 7.83-7.81 (m, 2H), 7.70-7.63 (m, 2H), 7.60-7.54 (m, 2H), 7.33-7.28 (m, 3H), 7.16-7.10 (m, 2H), 6.58 (d, J=8.4 Hz, 1H), 1.22 (s, 9H).

Step 2: To a solution of 5-(tert-butyl)-N-(diphenylmethylene)pyridin-2-amine (720 mg, 2.29 mmol, 1.00 eq) in tetrahydrofuran (7.50 mL) and methanol (7.50 mL) was added hydrochloric acid (2.00 M, 7.20 mL, 6.29 eq). The mixture was stirred at 25° C. for 2 h. The reaction mixture was concentrated under reduced pressure to remove methanol and extracted with ethyl acetate (3×10 mL). The combined aqueous phase layers were added saturated sodium carbonate solution to pH=8 and extracted with ethyl acetate (3×20 mL). The combined organic layers were washed with brine, dried over sodium sulfate, filtered and concentrated in vacuum to afford 5-(tert-butyl)pyridin-2-amine. ¹H NMR (400 MHz, DMSO-d₆) δ=7.89 (d, J=2.4 Hz, 1H), 7.40 (dd, J=2.6, 8.7 Hz, 1H), 6.38 (d, J=8.7 Hz, 1H), 5.62 (s, 2H), 1.21 (s, 9H).

Step 3: To a solution of 5-(tert-butyl)pyridin-2-amine (0.200 g, 1.33 mmol, 1.00 eq) and pyridine (316 mg, 3.99 mmol, 322 uL, 3.00 eq) in Acetonitrile (5.00 mL) was added phenyl carbonochloridate (313 mg, 2.00 mmol, 250 uL, 1.50 eq) at 0° C. The mixture was stirred at 25° C. for 12 h. The mixture was concentrated in vacuum. The crude product was purified by reversed-phase HPLC to afford phenyl (5-(tert-butyl)pyridin-2-yl)carbamate. ¹H NMR (400 MHz, DMSO-d₆) δ=10.6 (s, 1H), 8.35 (d, J=2.1 Hz, 1H), 7.83 (dd, J=2.6, 8.7 Hz, 1H), 7.78-7.70 (m, 1H), 7.45-7.40 (m, 2H), 7.29-7.24 (m, 1H), 7.21 (d, J=7.7 Hz, 2H), 1.29 (s, 9H).

Step 4: To a solution of 3-(6-(hydroxymethyl)-1-oxoisoindolin-2-yl)piperidine-2,6-dione VIII (53 mg, 193 umol, 1.00 eq) and phenyl (5-(tert-butyl)pyridin-2-yl)carbamate (62.7 mg, 232 umol, 1.20 eq) in dimethyl formamide (2.00 mL) was added sodium hydride (11.6 mg, 290 umol, 60% purity, 1.50 eq) at 0° C. The mixture was stirred at 0° C. for 1 h. The reaction was quenched by acetic acid (0.500 mL) slowly and then filtered and concentrated in vacuum. The residue was purified by prep-HPLC (column: Phenomenex Synergi C18 150*25 mm*10 um; mobile phase: water (0.225% Formic acid)-Acetonitrile; B %: 19%-49%, 10 min) and lyophilized. The crude product was purified by prep-HPLC and lyophilized to afford (2-(2,6-dioxopiperidin-3-yl)-3-oxoisoindolin-5-yl)methyl (5-(tert-butyl)pyridin-2-yl)carbamate 131. ¹H NMR (400 MHz, DMSO-d₆) δ=10.98 (br s, 1H), 10.25 (s, 1H), 8.29 (d, J=2.3 Hz, 1H), 7.82-7.77 (m, 2H), 7.76-7.72 (m, 1H), 7.69-7.65 (m, 1H), 7.65-7.61 (m, 1H), 5.28 (s, 2H), 5.13 (dd, J=5.1, 13.3 Hz, 1H), 4.50-4.42 (m, 1H), 4.38-4.29 (m, 1H), 2.97-2.86 (m, 1H), 2.60 (br dd, J=2.1, 15.1 Hz, 1H), 2.40 (dd, J=4.5, 13.2 Hz, 1H), 2.06-1.96 (m, 1H), 1.28 (s, 9H). MS (ESI) m/z 451.2 [M+H]⁺

### Compound 332:

Step 1: To a mixture of 1-chloro-2,3-difluoro-5-nitrobenzene (1.00 g, 5.17 mmol, 1.00 eq) and iron powder (1.44 g, 25.8 mmol, 5.00 eq) and ammonium chloride (1.38 g, 25.8 mmol, 5.00 eq) in methanol (16.0 mL) was added water (16.0 mL). The reaction mixture was stirred at 80° C. for 2 h. Then the reaction mixture was washed with methanol (80.0 mL) and then filtered. The filtrate was concentrated under reduced pressure to afford a residue. The residue was diluted with water (80.0 mL), and extracted with ethyl acetate (3×25.0 mL). Then the organic phase was washed with brine, dried over anhydrous sodium sulfate and concentrated under reduced pressure to afford 3-chloro-4,5-difluoroaniline. ¹H NMR (400 MHz, DMSO-d₆) δ=6.56-6.40 (m, 2H), 5.53 (s, 2H). MS (ESI) m/z 164.0 [M+H]⁺

Step 2: To a mixture of 3-chloro-4,5-difluoroaniline (1.00 g, 6.11 mmol, 1.00 eq) and pyridine (1.45 g, 18.3 mmol, 1.48 mL, 3.00 eq) in Acetonitrile (12.0 mL) was added phenyl carbonochloridate (1.34 g, 8.56 mmol, 1.07 mL, 1.40 eq) at 25° C. The reaction mixture was stirred at 25° C. for 12 h. The reaction mixture was concentrated under reduced pressure to afford a residue. The residue was diluted with water (80.0 mL) and extracted with ethyl acetate (3×20.0 mL). Then the organic phase was combined and concentrated under reduced pressure to afford a residue. The residue was purified by reverse phase HPLC and the desired eluent was lyophilized to afford phenyl (3-chloro-4,5-difluorophenyl)carbamate. ¹H NMR (400 MHz, DMSO-d₆) δ=9.32 (br s, 1H), 7.68-7.20 (m, 5H), 6.79-6.72 (m, 2H). MS (ESI) m/z 284.1 [M+H]⁺

Step 3: To a solution of phenyl (3-chloro-4,5-difluorophenyl)carbamate (50.7 mg, 179 umol, 1.40 eq) and 3-(6-(hydroxymethyl)-1-oxoisoindolin-2-yl)piperidine-2,6-dione VIII (35.0 mg, 128 umol, 1.00 eq) in dimethyl formamide (1.00 mL) was added sodium hydride (10.2 mg, 255 umol, 60% purity, 2.00 eq). The mixture was stirred at 25° C. for 1 h. The pH of the mixture was adjusted to 7 with hydrochloric acid (1 M) to give a solution. The solution was purified by Prep-HPLC and lyophilized to afford (2-(2,6-dioxopiperidin-3-yl)-3-oxoisoindolin-5-yl)methyl(3-chloro-4,5-difluorophenyl)carbamate 132. ¹H NMR (400 MHz, DMSO-d₆) δ=10.99 (br s, 1H), 10.23 (s, 1H), 7.79 (s, 1H), 7.70-7.66 (m, 1H), 7.66-7.62 (m, 1H), 7.51 (ddd, J=2.8, 6.4, 12.4 Hz, 1H), 7.47-7.43 (m, 1H), 5.29 (s, 2H), 5.12 (dd, J=5.2, 13.2 Hz, 1H), 4.51-4.44 (m, 1H), 4.38-4.31 (m, 1H), 2.97-2.85 (m, 1H), 2.63-2.58 (m, 1H), 2.47-2.34 (m, 1H), 2.06-1.97 (m, 1H). MS (ESI) m/z 464.1 [M+H]⁺

### Compound 333:

Step 1: To a mixture of 3-Fluoro-4-tolylamine (250 mg, 2.00 mmol, 229 uL, 1.00 eq) and pyridine (474 mg, 5.99 mmol, 484 uL, 3.00 eq) in Acetonitrile (3.00 mL) was added phenyl carbonochloridate (437 mg, 2.80 mmol, 350 uL, 1.40 eq) at 25° C. The reaction mixture was stirred at 25° C. for 12 h. The reaction mixture was concentrated under reduced pressure to afford a residue. The residue was purified by Reversed-Phase Flash and lyophilized to afford phenyl (3-fluoro-4-methylphenyl)carbamate. ¹H NMR (400 MHz, DMSO-d₆) δ=9.31 (s, 1H), 7.47-7.36 (m, 5H), 7.22-7.16 (m, 3H), 2.17 (d, J=1.6 Hz, 3H). MS (ESI) m/z 246.0 [M+H]⁺

Step 2: To a solution of phenyl (3-fluoro-4-methylphenyl)carbamate (100 mg, 407 umol, 1.40 eq) and 3-(6-(hydroxymethyl)-1-oxoisoindolin-2-yl)piperidine-2,6-dione VIII (80.0 mg, 292 umol, 1.00 eq) in dimethyl formamide (1.20 mL) was added sodium hydride (23.3 mg, 583 umol, 60% purity, 2.00 eq). The mixture was stirred at 25° C. for 1 h. The pH of the mixture was adjusted to 7 with hydrochloric acid (1 M) to give a solution. The solution was purified by Prep-HPLC and lyophilized to afford (2-(2,6-dioxopiperidin-3-yl)-3-oxoisoindolin-5-yl)methyl (3-fluoro-4-methylphenyl)carbamate 133. ¹H NMR (400 MHz, DMSO-d₆) δ=10.99 (s, 1H), 9.93 (s, 1H), 7.79 (s, 1H), 7.72-7.60 (m, 2H), 7.33 (br d, J=12.0 Hz, 1H), 7.22-7.09 (m, 2H), 5.27 (s, 2H), 5.13 (dd, J=5.2, 13.2 Hz, 1H), 4.51-4.43 (m, 1H), 4.38-4.30 (m, 1H), 2.97-2.86 (m, 1H), 2.60 (td, J=2.0, 15.2 Hz, 1H), 2.40 (dq, J=4.4, 13.2 Hz, 1H), 2.15 (d, J=1.2 Hz, 3H), 2.05-1.96 (m, 1H). MS (ESI) m/z 426.2 [M+H]⁺

### Compound 334:

Step 1: To a mixture of 4-bromo-3,5-difluoroaniline (1.00 g, 4.81 mmol, 1.00 eq) and phenylboronic acid (703 mg, 5.77 mmol, 1.20 eq) in dioxane (15.0 mL) was added tetrakis(triphenylphosphine)palladium(0) (1.11 g, 962 umol, 0.200 eq) and potassium carbonate (997 mg, 7.21 mmol, 1.50 eq) at 25° C. under nitrogen. The mixture was stirred at 100° C. for 12 h. The reaction mixture was diluted with water (20.0 mL) and extracted with ethyl acetate (3×20.0 mL). The combined organic phase was separated, washed with brine (2×10.0 mL), dried over sodium sulfate, filtered and concentrated under reduced pressure to give a residue. The residue was purified by silica gel chromatography to afford 2,6-difluoro-[1,1′-biphenyl]-4-amine. MS (ESI) m/z 206.2 [M+H]⁺.

Step 2: To a mixture of 2,6-difluoro-[1,1′-biphenyl]-4-amine (400 mg, 1.95 mmol, 294 uL, 1.00 eq) and phenyl carbonochloridate (336 mg, 2.14 mmol, 269 uL, 1.10 eq) in Acetonitrile (2.00 mL) was added pyridine (463 mg, 5.85 mmol, 472 uL, 3.00 eq) at 0° C. The mixture was stirred at 25° C. for 2 h. The reaction mixture was diluted with water (20.0 mL) and extracted with ethyl acetate (3×20.0 mL). The organic phase was combined, washed with brine (20.0 mL), dried over sodium sulfate, filtered and concentrated under reduced pressure to give a residue. The residue was purified by reversed phase-HPLC to afford phenyl (2,6-difluoro-[1,1′-biphenyl]-4-yl)carbamate. MS (ESI) m/z 326.1 [M+H]⁺

Step 3: To a mixture of phenyl (2,6-difluoro-[1,1′-biphenyl]-4-yl)carbamate (65.2 mg, 201 umol, 1.10 eq) and 3-(6-(hydroxymethyl)-1-oxoisoindolin-2-yl)piperidine-2,6-dione VIII (50.0 mg, 182 umol, 1.00 eq) in dimethyformamide (2.00 mL) was added sodium hydride (14.6 mg, 365 umol, 60% purity, 2.00 eq) at 0° C. under nitrogen. The mixture was stirred at 25° C. for 1 h. The mixture was quenched with 1 M hydrochloric acid and filtered. The filter cake was triturated with water and filtered. The filter cake was washed with acetonitrile and dried to afford (2-(2,6-dioxopiperidin-3-yl)-3-oxoisoindolin-5-yl)methyl(2,6-difluoro-[1,1′-biphenyl]-4-yl)carbamate 134. ¹H NMR (400 MHz, DMSO-d₆) δ=11.00 (br s, 1H), 10.34 (br s, 1H), 7.82 (s, 1H), 7.74-7.62 (m, 2H), 7.54-7.46 (m, 2H), 7.44 (br s, 3H), 7.30 (br d, J=9.6 Hz, 2H), 5.34 (s, 2H), 5.18-5.08 (m, 1H), 4.54-4.46 (m, 1H), 4.42-4.32 (m, 1H), 2.96-2.88 (m, 1H), 2.68-2.62 (m, 1H), 2.38-2.32 (m, 1H), 2.08-1.98 (m, 1H). MS (ESI) m/z 506.2 [M+H]⁺

### Compound 335:

Step 1: To a solution of 4-bromo-2-fluoroaniline (3.00 g, 15.8 mmol, 1.00 eq) and 4,4,4′,4′,5,5,5′,5′-octamethyl-2,2′-bi(1,3,2-dioxaborolane) (6.01 g, 23.7 mmol, 1.50 eq) in dioxane (30.0 mL) was added potassium acetate (4.65 g, 47.4 mmol, 3.00 eq) and [1,1′-bis(diphenylphosphino) ferrocene]dichloropalladium(II) (1.29 g, 1.58 mmol, 0.100 eq) under nitrogen atmosphere. The mixture was stirred at 80° C. for 12 h under nitrogen atmosphere. The mixture was diluted with water (80.0 mL) and extracted with ethyl acetate (3×60.0 mL). The combined organic layer were washed with saturated brine (20.0 mL), dried with anhydrous sodium sulfate, filtered and concentrated in vacuum. The residue was purified by column chromatography on silica gel to give 2-fluoro-4-(4,4,5,5-tetramethyl-1,3,2-dioxaborolan-2-yl)aniline. ¹H NMR (400 MHz, DMSO-d₆) δ=7.23-7.09 (m, 2H), 6.73 (dd, J=8.0, 8.8 Hz, 1H), 5.55 (s, 2H), 1.24 (s, 12H).

Step 2: To a solution of 2-fluoro-4-(4,4,5-trimethyl-1,3,2-dioxaborolan-2-yl)aniline (2.80 g, 11.8 mmol, 1.00 eq) and 2-bromopyridine (2.80 g, 17.7 mmol, 1.69 mL, 1.50 eq) in dioxane (20.0 mL) and water (5.00 mL) was added [1,1′-bis(diphenylphosphino)ferrocene]dichloropalladium(II) (964 mg, 1.18 mmol, 0.100 eq) and tripotassium phosphate (7.52 g, 35.4 mmol, 3.00 eq) in portions. The mixture was stirred at 80° C. for 12 h under nitrogen atmosphere. The mixture was diluted with water (80.0 mL) and extracted with ethyl acetate (3×60.0 mL). The combined organic layers were washed with saturated brine (20.0 mL), dried with anhydrous sodium sulfate, filtered and concentrated in vacuum. The residue was purified by column chromatography on silica gel to give 2-fluoro-4-(2-pyridyl)aniline. ¹H NMR (400 MHz, DMSO-d₆) δ=8.55 (br d, J=4.5 Hz, 1H), 7.82-7.72 (m, 3H), 7.67 (br d, J=8.3 Hz, 1H), 7.23-7.15 (m, 1H), 6.84 (t, J=8.4 Hz, 1H), 5.49 (s, 2H).

Step 3: To a solution of 2-fluoro-4-(pyridin-2-yl)aniline (500 mg, 2.66 mmol, 1.00 eq) and phenyl carbonochloridate (624 mg, 3.99 mmol, 499 uL, 1.50 eq) in Acetonitrile (5.00 mL) was added pyridine (630 mg, 7.97 mmol, 643 uL, 3.00 eq) dropwise. The mixture was stirred at 25° C. for 12 h. The mixture was concentrated to give crude product. The crude product was purified by reversed-phase HPLC and the desired fraction was collected and lyophilized to give phenyl (2-fluoro-4-(pyridin-2-yl)phenyl)carbamate. ¹H NMR (400 MHz, DMSO-d₆) δ=10.14 (br s, 1H), 8.70-8.63 (m, 1H), 8.02-7.94 (m, 3H), 7.88 (brt, J=7.7 Hz, 2H), 7.49-7.43 (m, 5H), 7.25 (br s, 1H).

Step 4: To a mixture of phenyl (2-fluoro-4-(pyridin-2-yl)phenyl)carbamate (108 mg, 350 umol, 1.20 eq) and 3-(6-(hydroxymethyl)-1-oxoisoindolin-2-yl)piperidine-2,6-dione VIII (80.0 mg, 292 umol, 1.00 eq) in dimethyl formamide (2.00 mL) was added sodium hydride (17.5 mg, 438 umol, 60% purity, 1.50 eq) in portions at 0° C. The mixture was stirred at 15° C. for 2 h. The mixture was quenched with 1 M hydrochloric acid and filtered. The filtrate was purified by prep-HPLC and lyophilized to afford (2-(2,6-dioxopiperidin-3-yl)-3-oxoisoindolin-5-yl)methyl (2-fluoro-4-(pyridin-2-yl)phenyl)carbamate 135. ¹H NMR (400 MHz, DMSO-d₆) δ=11.00 (s, 1H), 9.74 (s, 1H), 8.65 (dd, J=0.9, 4.8 Hz, 1H), 8.14 (s, 1H), 8.00-7.86 (m, 5H), 7.84 (s, 1H), 7.73-7.68 (m, 1H), 7.66-7.62 (m, 1H), 7.36 (ddd, J=0.9, 4.8, 7.4 Hz, 1H), 5.31 (s, 2H), 5.13 (dd, J=5.1, 13.3 Hz, 1H), 4.53-4.44 (m, 1H), 4.42-4.29 (m, 1H), 2.99-2.85 (m, 1H), 2.61 (br d, J=16.6 Hz, 1H), 2.46-2.35 (m, 1H), 2.09-1.95 (m, 1H). MS (ESI) m/z 489.1 [M+H]⁺

### Compound 336:

Step 1: To a mixture of 4-bromo-3-fluoroaniline (2.00 g, 10.5 mmol, 1.00 eq), phenylboronic acid (1.93 g, 15.8 mmol, 1.50 eq), potassium carbonate (4.36 g, 31.6 mmol, 3.00 eq) in dioxane (20.0 mL) and water (2.00 mL) was added [1,1′-bis(diphenylphosphino)ferrocene]dichloropalladium(II) (1.16 g, 1.58 mmol, 0.150 eq). The mixture was stirred at 100° C. for 12 h. The mixture was concentrated to give a residue, which was purified by flash silica gel chromatography to give 2-fluoro-[1,1′-biphenyl]-4-amine. MS (ESI) m/z 188.1 [M+H]⁺

Step 2: To a mixture of 2-fluoro-[1,1′-biphenyl]-4-amine (2.00 g, 10.7 mmol, 1.00 eq) in Acetonitrile (20.0 mL) was added pyridine (2.54 g, 32.1 mmol, 2.59 mL, 3.00 eq) and phenyl carbonochloridate (1.84 g, 11.7 mmol, 1.47 mL, 1.10 eq) at 0° C. The mixture was stirred at 0° C. for 1 h. The reaction mixture was concentrated under reduced pressure to give a residue, which was purified by column chromatography to give phenyl(2-fluoro-[1,1′-biphenyl]-4-yl)carbamate. MS (ESI) m/z 308 [M+H]⁺

Step 3: To a mixture of phenyl (2-fluoro-[1,1′-biphenyl]-4-yl)carbamate (107 mg, 350 umol, 1.20 eq) and 3-(6-(hydroxymethyl)-1-oxoisoindolin-2-yl)piperidine-2,6-dione VIII (80.0 mg, 291 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h under nitrogen atmosphere. The mixture was adjusted to pH=4 with hydrochloric acid (1 M) to give a solution. The solution was purified by Prep-HPLC and lyophilized to afford (2-(2,6-dioxopiperidin-3-yl)-3-oxoisoindolin-5-yl)methyl (2-fluoro-[1,1′-biphenyl]-4-yl) carbamate 136. ¹H NMR (400 MHz, DMSO-d₆) δ=10.98 (br s, 1H), 10.17 (br s, 1H), 8.50 (s, 1H), 7.82 (s, 1H), 7.72-7.68 (m, 1H), 7.67-7.62 (m, 1H), 7.53-7.43 (m, 6H), 7.35 (brt, J=9.3 Hz, 2H), 5.31 (s, 2H), 5.13 (br dd, J=4.6, 13.1 Hz, 1H), 4.51-4.44 (m, 1H), 4.38-4.31 (m, 1H), 2.97-2.86 (m, 1H), 2.61 (br d, J=16.3 Hz, 1H), 2.41 (br dd, J=3.5, 13.0 Hz, 1H), 2.05-1.98 (m, 1H). MS (ESI) m/z 488.3 [M+H]⁺

### Compound 337:

Step 1: To a solution of 4-bromo-3-methylaniline (2.00 g, 10.8 mmol, 1.00 eq), phenylboronic acid (1.97 g, 16.1 mmol, 1.50 eq) and potassium phosphate (6.85 g, 32.3 mmol, 3.00 eq) in dioxane (20.0 mL) and water (5.00 mL) was added [1,1′-bis(diphenylphosphino)ferrocene]dichloropalladium(II) (787 mg, 1.07 mmol, 0.100 eq) under nitrogen atmosphere. The mixture was stirred at 80° C. for 12 h. The reaction mixture was cooled to room temperature, ethyl acetate (40.0 mL) and water (60.0 mL) were added and organic layers were separated. The aqueous phase was extracted with ethyl acetate (3×30.0 mL). Combined extracts were washed with brine (60.0 mL), dried over sodium sulfate, filtered and concentrated under vacuum. The residue was purified by column chromatography on silica gel to give 2-methyl-[1,1′-biphenyl]-4-amine. ¹H NMR (400 MHz, DMSO-d₆) δ=7.42-7.31 (m, 2H), 7.29-7.21 (m, 3H), 6.87 (d, J=8.0 Hz, 1H), 6.54-6.41 (m, 2H), 5.04 (s, 2H), 2.12 (s, 3H). MS (ESI) m/z 184.0 [M+H]

Step 2: To a mixture of 2-methyl-[1,1′-biphenyl]-4-amine (500 mg, 2.73 mmol, 1.00 eq) and pyridine (1.08 g, 13.6 mmol, 1.10 mL, 5.00 eq) in Acetonitrile (10.0 mL) was added phenyl carbonochloridate (555 mg, 3.55 mmol, 444 uL, 1.30 eq) dropwise at 0° C. The mixture was then stirred at 20° C. for 2 h. The mixture was concentrated in vacuum to give crude product which was purified by reversed-phase HPLC. The desired fraction was collected and lyophilized to give phenyl (2-methyl-[1,1′-biphenyl]-4-yl)carbamate. MS (ESI) m/z 304.2 [M+H]⁺

Step 3: To a solution of 3-(6-(hydroxymethyl)-1-oxoisoindolin-2-yl)piperidine-2,6-dione VIII (140 mg, 510 umol, 1.00 eq) and phenyl (2-methyl-[1,1′-biphenyl]-4-yl)carbamate (185 mg, 613 umol, 1.20 eq) in dimethyl formamide (1.00 mL) was added sodium hydride (40.8 mg, 1.02 mmol, 60% purity, 2.00 eq) at 0° C. The mixture was stirred at 20° C. for 1 h. The mixture was quenched by 1 M hydrochloric acid (1.00 mL) and filtered. The filtrate was purified by prep-HPLC and lyophilized to give (2-(2,6-dioxopiperidin-3-yl)-3-oxoisoindolin-5-yl)methyl(2-methyl-[1,1′-biphenyl]-4-yl)carbamate 137. ¹H NMR (400 MHz, DMSO-d₆) δ=11.00 (s, 1H), 9.83 (s, 1H), 7.82 (s, 1H), 7.73-7.68 (m, 1H), 7.67-7.63 (m, 1H), 7.45-7.40 (m, 3H), 7.39 (br s, 1H), 7.36-7.33 (m, 1H), 7.33-7.29 (m, 2H), 7.13 (d, J=8.2 Hz, 1H), 5.29 (s, 2H), 5.14 (dd, J=5.1, 13.3 Hz, 1H), 4.53-4.43 (m, 1H), 4.40-4.29 (m, 1H), 2.98-2.87 (m, 1H), 2.64-2.58 (m, 1H), 2.41 (br dd, J=4.4, 13.1 Hz, 1H), 2.20 (s, 3H), 2.06-1.98 (m, 1H). MS (ESI) m/z 484.2 [M+H]⁺

### Compound 338:

Step 1: A mixture of aniline (300 mg, 3.22 mmol, 294 uL, 1.00 eq), phenyl carbonochloridate (555 mg, 3.54 mmol, 444 uL, 1.10 eq) and pyridine (1.27 g, 16.1 mmol, 1.30 mL, 5.00 eq) in Acetonitrile (3.00 mL) was stirred at 25° C. for 2 h. The mixture was concentrated to give crude product. The crude product was purified by reversed phase column chromatography. The desired fraction was collected and lyophilized to afford phenyl phenylcarbamate. MS (ESI) m/z 214.1 [M+H]⁺

Step 2: To a mixture of phenyl phenylcarbamate (42.8 mg, 201 umol, 1.10 eq) and 3-(6-(hydroxymethyl)-1-oxoisoindolin-2-yl)piperidine-2,6-dione (50.0 mg, 183 umol, 1.00 eq) in dimethyformamide (2.00 mL) was added sodium hydride (14.6 mg, 365 umol, 60% purity, 2.00 eq) at 0° C. The mixture was stirred at 25° C. for 1 h. The mixture was quenched with 1 M hydrochloric acid and filtered. The filtrate was purified by Prep-HPLC and the desired fraction was collected and lyophilized to afford (2-(2,6-dioxopiperidin-3-yl)-3-oxoisoindolin-5-yl)methyl phenylcarbamate 138. ¹H NMR (400 MHz, DMSO-d₆) δ=11.00 (s, 1H), 9.81 (s, 1H), 7.81 (s, 1H), 7.72-7.62 (m, 2H), 7.48 (br d, J=7.6 Hz, 2H), 7.28 (t, J=8.0 Hz, 2H), 7.00 (t, J=7.6 Hz, 1H), 5.28 (s, 2H), 5.14 (dd, J=5.2, 13.6 Hz, 1H), 4.54-4.44 (m, 1H), 4.38-4.30 (m, 1H), 2.98-2.86 (m, 1H), 2.68-2.56 (m, 1H), 2.44-2.32 (m, 1H), 2.12-1.94 (m, 1H). MS (ESI) m/z 394.2 [M+H]⁺

### Compound 339:

Step 1: To a solution of 3-(trifluoromethyl)isothiazol-5-amine (100 mg, 594 umol, 1.00 eq) in ethyl acetate (2.00 mL), tetrahydrofuran (0.500 mL) and water (0.500 mL) was added sodium carbonate (37.8 mg, 356 umol, 0.600 eq) and phenyl carbonochloridate (102 mg, 654 umol, 81.9 uL, 1.10 eq) at 0° C. The mixture was stirred at 25° C. for 1 h. The mixture was diluted with water (10.0 mL), and then extracted with ethyl acetate (2×10.0 mL). The combined organic phase was washed with brine (20.0 mL), dried over anhydrous sodium sulfate, filtered and concentrated in vacuum. The residue was purified by flash silica gel chromatography to give phenyl (3-(trifluoromethyl)isothiazol-5-yl)carbamate. ¹H NMR (400 MHz, CDCl₃) δ=8.20 (br s, 1H), 7.41-7.30 (m, 3H), 7.28-7.20 (m, 1H), 7.17-7.10 (m, 2H), 6.93 (s, 1H).

Step 2: To a solution of phenyl (3-(trifluoromethyl)isothiazol-5-yl)carbamate (150 mg, 520 umol, 1.20 eq) in dimethylformamide (2.00 mL) was added N,N-diisopropylethylamine (168 mg, 1.30 mmol, 226 uL, 3.00 eq) and 1-hydroxybenzotriazole (70.3 mg, 520 umol, 1.20 eq) and 3-(6-(hydroxymethyl)-1-oxoisoindolin-2-yl) piperidine-2,6-dione (119 mg, 433 umol, 1.00 eq) at 25° C. The mixture was stirred at 40° C. for 2 h. The mixture was diluted with water (10.0 mL), then extracted with ethyl acetate (2×20.0 mL). The combined organic phase was washed with water (20.0 mL) and brine (20.0 mL), dried over anhydrous sodium sulfate, filtered and concentrated in vacuum. The crude product was purified by prep-HPLC to give (2-(2,6-Dioxo-piperidin-3-yl)-3-oxoisoindolin-5-yl)methyl(3-(trifluoro-methyl)isothiazol-5-yl)carbamate 139. ¹H NMR (400 MHz, DMSO-d₆) δ=12.13 (s, 1H), 11.00 (s, 1H), 7.83 (s, 1H), 7.75-7.70 (m, 1H), 7.69-7.64 (m, 1H), 7.06 (s, 1H), 5.42 (s, 2H), 5.13 (dd, J=5.2, 13.3 Hz, 1H), 4.53-4.44 (m, 1H), 4.40-4.32 (m, 1H), 2.92 (ddd, J=5.6, 13.6, 17.4 Hz, 1H), 2.61 (br d, J=17.2 Hz, 1H), 2.41 (dd, J=4.4, 13.2 Hz, 1H), 2.08-1.97 (m, 1H). ¹⁹F NMR (400 MHz, DMSO-d₆) δ=−63.62 (s, 1F). MS (ESI) m/z 469.0 [M+H]⁺

### Compound 340:

Step 1: To a solution of 3-nitrophenol (1.00 g, 7.19 mmol, 1.43 mL, 1.00 eq) in acetone (20.0 mL) was added 1,2-dibromoethane (2.00 g, 10.6 mmol, 1.48 eq) and potassium carbonate (3.00 g, 21.7 mmol, 3.02 eq). The mixture was stirred at 80° C. for 12 h. The mixture was concentrated under reduced pressure. The residue was purified by column chromatography on silica gel to give 1-(2-bromoethoxy)-3-nitrobenzene. ¹H NMR (400 MHz, CDCl₃) δ=7.88 (ddd, J=0.8, 2.1, 8.1 Hz, 1H), 7.75 (t, J=2.3 Hz, 1H), 7.47 (t, J=8.2 Hz, 1H), 7.29-7.26 (m, 1H), 4.39 (t, J=6.1 Hz, 2H), 3.69 (t, J=6.1 Hz, 2H).

Step 2: To a solution of 1-(2-bromoethoxy)-3-nitrobenzene (1.20 g, 4.88 mmol, 1.00 eq) in tetrahydrofuran (20.0 mL) was added potassium tert-butoxide (650 mg, 5.79 mmol, 1.19 eq). The mixture was stirred at 20° C. for 0.5 h. The mixture was diluted with ethyl acetate and water. The organic layer was separated and concentrated under reduced pressure to give 1-nitro-3-(vinyloxy)benzene. ¹H NMR (400 MHz, CDCl₃) δ=8.01-7.94 (m, 1H), 7.88 (t, J=2.3 Hz, 1H), 7.52 (t, J=8.2 Hz, 1H), 7.36 (td, J=1.2, 8.2 Hz, 1H), 6.69 (dd, J=6.0, 13.6 Hz, 1H), 4.94 (dd, J=1.9, 13.6 Hz, 1H), 4.66 (dd, J=2.0, 6.0 Hz, 1H).

Step 3: To a solution of 1-nitro-3-(vinyloxy)benzene (500 mg, 3.03 mmol, 1.00 eq) in xylene (10.0 mL) was added sodium fluoride (75.0 mg, 1.79 mmol, 0.59 eq) and trimethylsilyl 2,2-difluoro-2-(fluorosulfonyl)acetate (1.50 g, 5.99 mmol, 1.18 mL, 1.98 eq). The mixture was stirred at 120° C. for 12 h under nitrogen atmosphere. After being cooled to room temperature, the mixture was diluted with ethyl acetate and water. The organic layer was separated and concentrated to give a residue. The residue was purified by column chromatography on silica gel to give 1-(2,2-difluorocyclopropoxy)-3-nitrobenzene. ¹H NMR (400 MHz, CDCl₃) δ=7.97-7.91 (m, 1H), 7.87 (t, J=2.3 Hz, 1H), 7.51 (t, J=8.3 Hz, 1H), 7.34 (ddd, J=0.8, 2.5, 8.3 Hz, 1H), 4.21-4.14 (m, 1H), 2.00-1.86 (m, 1H), 1.76-1.67 (m, 1H).

Step 4: To a solution of 1-(2,2-difluorocyclopropoxy)-3-nitrobenzene (520 mg, 2.42 mmol, 1.00 eq) in methanol (20.0 mL) and water (4.00 mL) was added iron powder (680 mg, 12.2 mmol, 5.04 eq) and ammonium chloride (1.04 g, 19.4 mmol, 8.04 eq). The mixture was stirred at 50° C. for 1 h. After being cooled to room temperature, the mixture was concentrated under reduced pressure. The residue was diluted with ethyl acetate and water. The organic layer was separated and concentrated under reduced pressure to give 3-(2,2-difluorocyclopropoxy)aniline. ¹H NMR (400 MHz, CDCl₃) δ=7.11 (t, J=8.1 Hz, 1H), 6.45-6.42 (m, 1H), 6.39 (ddd, J=0.7, 2.1, 7.9 Hz, 1H), 6.36-6.33 (m, 1H), 4.08-3.98 (m, 1H), 3.74 (br s, 2H), 1.86-1.74 (m, 1H), 1.67-1.59 (m, 1H).

Step 5: To a solution of 3-(2,2-difluorocyclopropoxy)aniline (200 mg, 1.08 mmol, 1.00 eq) in Acetonitrile (20.0 mL) was added pyridine (254 mg, 3.22 mmol, 3.00 eq) and phenyl carbonochloridate (187 mg, 1.20 mmol, 1.11 eq) at 0° C. The mixture was stirred at 0° C. for 1 h. The mixture was concentrated under reduced pressure. The residue was purified by column chromatography on silica gel to give phenyl (3-(2,2-difluorocyclopropoxy)phenyl)carbamate.

MS (ESI) m/z 306.1 [M+H]⁺

Step 6: To a solution of 3-(6-(hydroxymethyl)-1-oxoisoindolin-2-yl)piperidine-2,6-dione VIII (90.0 mg, 328 umol, 1.00 eq) in dimethyl formamide (2.00 mL) was added (3-(2,2-difluorocyclopropoxy)phenyl)carbamate (110 mg, 360 umol, 1.10 eq) and sodium hydride (26.0 mg, 650 umol, 60% purity, 1.98 eq). The mixture was stirred at 0° C. for 1 h. The mixture was quenched with hydrochloric acid (1 M, 1.00 mL) to give solution. The solution was purified by Prep-HPLC and the desired fraction was collected and lyophilized to afford (3-(2,2-difluorocyclopropoxy)phenyl) carbamate 140. ¹H NMR (400 MHz, DMSO-d₆) δ=11.00 (br s, 1H), 9.92 (s, 1H), 7.80 (s, 1H), 7.72-7.60 (m, 2H), 7.31-7.21 (m, 2H), 7.12 (br d, J=8.2 Hz, 1H), 6.69 (dd, J=1.9, 8.2 Hz, 1H), 5.28 (s, 2H), 5.13 (dd, J=5.1, 13.3 Hz, 1H), 4.51-4.40 (m, 2H), 4.38-4.31 (m, 1H), 2.99-2.84 (m, 1H), 2.65-2.58 (m, 1H), 2.41 (br dd, J=4.6, 13.1 Hz, 1H), 2.08-1.98 (m, 2H), 1.83-1.71 (m, 1H). MS (ESI) m/z 486.1 [M+H]⁺

### Compound 341:

Step 1: A mixture of 1-chloro-4-fluoro-2-methyl-5-nitrobenzene (500 mg, 2.64 mmol, 1.00 eq), ammonium chloride (988 mg, 18.5 mmol, 7.00 eq) and iron powder (1.03 g, 18.5 mmol, 7.00 eq) in methanol (10.0 mL) and water (2.00 mL) was stirred at 80° C. for 2 h. The resulting mixture was filtered over celite, and the filtrate was concentrated under reduced pressure to give 5-chloro-2-fluoro-4-methylaniline.

Step 2: A mixture of phenyl carbonochloridate (236 mg, 1.50 mmol, 188 uL, 1.20 eq), 5-chloro-2-fluoro-4-methylaniline (200 mg, 1.25 mmol, 1.00 eq) and pyridine (298 mg, 3.76 mmol, 304 uL, 3.00 eq) in Acetonitrile (10.0 mL) was stirred at 25° C. for 12 h. The mixture was concentrated in vacuum. The crude product was purified by reversed-phase HPLC and the desired fraction was collected and lyophilized to give phenyl (5-chloro-2-fluoro-4-methylphenyl)carbamate. MS (ESI) m/z 280.1 [M+H]⁺

Step 3: To a mixture of phenyl(5-chloro-2-fluoro-4-methylphenyl)carbamate (122 mg, 438 umol, 1.20 eq) and 3-(6-(hydroxymethyl)-1-oxoisoindolin-2-yl)piperidine-2,6-dione VIII (100 mg, 365 umol, 1.00 eq) in dimethyl formamide (2.00 mL) was added sodium hydride (29.2 mg, 729 umol, 60% purity, 2.00 eq) under nitrogen atmosphere. Then the mixture was stirred at 0° C. for 2 h. The residue was diluted with ethyl acetate (30.0 mL) and water (30.0 mL). The organic layer was separated and the aqueous phase was extracted with ethyl acetate (3×30.0 mL). The organic layer was dried over sodium sulfate, filtered and concentrated in vacuum. The residue was purified by prep-HPLC and the desired fraction was collected and lyophilized. The lyophilized residue was purified again by Prep-HPLC and the desired fraction was collected and lyophilized to give (2-(2,6-dioxopiperidin-3-yl)-3-oxoisoindolin-5-yl)methyl (5-chloro-2-fluoro-4-methylphenyl)carbamate 141. ¹H NMR (400 MHz, DMSO-d₆) δ=10.99 (br s, 1H), 9.67 (br s, 1H), 7.80 (s, 1H), 7.75 (br d, J=7.1 Hz, 1H), 7.69-7.61 (m, 2H), 7.29 (d, J=11.4 Hz, 1H), 5.27 (s, 2H), 5.17-5.09 (m, 1H), 4.51-4.43 (m, 1H), 4.38-4.28 (m, 1H), 2.98-2.85 (m, 1H), 2.64-2.57 (m, 1H), 2.44-2.37 (m, 1H), 2.27 (s, 3H), 2.06-1.96 (m, 1H). MS (ESI) m/z 460.1 [M+H]⁺

### Compound 342:

Step 1: To a mixture of 4-(tert-butyl)aniline (2.00 g, 13.4 mmol, 2.12 mL, 1.00 eq) in dichloromethane (20.0 mL) was added acetic anhydride (1.64 g, 16.1 mmol, 1.51 mL, 1.20 eq) dropwise at 0° C. The mixture was stirred at 0° C. for 15 min. The reaction was quenched with water (100 mL) and extracted with ethyl acetate (3×30.0 mL). The combined organic layer was washed with brine (20.0 mL), dried over anhydrous sodium sulfate and concentrated in vacuum to afford N-(4-(tert-butyl)phenyl)acetamide. ¹H NMR (400 MHz, CDCl₃) δ=7.43-7.37 (m, 2H), 7.35-7.31 (m, 2H), 2.16 (s, 3H), 1.30 (s, 9H). MS (ESI) m/z 192.2 [M+H]⁺

Step 2: To a mixture of N-(4-(tert-butyl)phenyl)acetamide (1.00 g, 5.23 mmol, 1.00 eq) in Acetonitrile (10.0 mL) was added Selectfluor (1.85 g, 5.23 mmol, 1.00 eq) in one portion. The mixture was stirred at 80° C. for 16 h under nitrogen atmosphere. The reaction mixture was cooled to room temperature and diluted with water (50 mL), extracted with ethyl acetate (3×30 mL). Combined organic layer was washed with brine (60.0 mL), dried over anhydrous sodium sulfate filtered, and concentrated under vacuum. The residue was purified by column chromatography on silica gel to afford N-(4-(tert-butyl)-2-fluorophenyl) acetamide. ¹H NMR (400 MHz, CDCl₃) δ=8.15 (t, J=8.6 Hz, 1H), 7.17-7.04 (m, 2H), 2.21 (s, 3H), 1.29 (s, 9H). MS (ESI) m/z 210.2[M+H]⁺

Step 3: To a solution of N-(4-(tert-butyl)-2-fluorophenyl)acetamide (300 mg, 1.43 mmol, 1.00 eq) in ethanol (4.00 mL) was added hydrochloric acid (12 M, 2.00 mL, 16.7 eq) dropwise. The mixture was stirred at 80° C. for 2 h. The reaction was quenched with saturated sodium hydrogencarbonate solution and extracted with ethyl acetate. The combined organic layer was washed with brine, dried over anhydrous sodium sulfate, filtered and concentrated in vacuum to afford 4-(tert-butyl)-2-fluoroaniline. MS (ESI) m/z 168.0[M+H]⁺

Step 4: To a mixture of 4-(tert-butyl)-2-fluoroaniline (110 mg, 658 umol, 1.00 eq) and pyridine (260 mg, 3.29 mmol, 265 uL, 5.00 eq) in Acetonitrile (5.00 mL) was added phenyl carbonochloridate (154 mg, 987 umol, 124 uL, 1.50 eq) dropwise at 0° C. The mixture was stirred at 20° C. for 2 h. The reaction was concentrated to give crude product which was purified by reversed-phase HPLC to afford phenyl (4-(tert-butyl)-2-fluorophenyl)carbamate. MS (ESI) m/z 288.2 [M+H]⁺

Step 5: To a solution of 3-(6-(hydroxymethyl)-1-oxoisoindolin-2-yl)piperidine-2,6-dione VIII (80.0 mg, 292 umol, 1.00 eq) and phenyl (4-(tert-butyl)-2-fluorophenyl)carbamate (109 mg, 379 umol, 1.30 eq) in dimethyl formamide (1.00 mL) was added sodium hydride (23.3 mg, 583 umol, 60% purity, 2.00 eq) at 0° C. The mixture was stirred at 20° C. for 0.5 h. The mixture was quenched by 1 M hydrochloric acid (1.00 mL) slowly and purified by Prep-HPLC. The desired fraction was collected and lyophilized to afford (2-(2,6-dioxopiperidin-3-yl)-3-oxoisoindolin-5-yl)methyl (4-(tert-butyl)-2-fluorophenyl)carbamate 142. ¹H NMR (400 MHz, DMSO-d₆) δ=11.00 (s, 1H), 9.42 (br s, 1H), 7.80 (s, 1H), 7.72-7.59 (m, 2H), 7.52 (brt, J=8.3 Hz, 1H), 7.26-7.11 (m, 2H), 5.26 (s, 2H), 5.13 (dd, J=5.0, 13.3 Hz, 1H), 4.53-4.42 (m, 1H), 4.39-4.29 (m, 1H), 2.97-2.87 (m, 1H), 2.61 (br d, J=17.8 Hz, 1H), 2.44-2.35 (m, 1H), 2.06-1.98 (m, 1H), 1.26 (s, 9H). MS (ESI) m/z 468.1 [M+H]⁺

### Compound 343:

Step 1: To a solution of 5-phenylpyridin-2-amine (500 mg, 2.94 mmol, 1.00 eq) in Acetonitrile (5.00 mL) and dimethyl formamidie (2.00 mL) was added pyridine (1.16 g, 14.7 mmol, 1.19 mL, 5.00 eq) and phenyl carbonochloridate (690 mg, 4.41 mmol, 552 uL, 1.50 eq). The mixture was stirred at 25° C. for 1 h. The mixture was filtered to give filtrate. The filtrate was concentrated and purified by reversed phase column chromatography. The desired fraction was collected and concentrated to give phenyl (5-phenylpyridin-2-yl)carbamate. MS (ESI) m/z 291.1 [M+H]⁺

Step 2: To a solution of 3-(6-(hydroxymethyl)-1-oxoisoindolin-2-yl)piperidine-2,6-dione VIII (90.0 mg, 328 umol, 1.00 eq) in dimethyl formamide (1.00 mL) was added phenyl (5-phenylpyridin-2-yl)carbamate (190 mg, 656 umol, 2.00 eq) and sodium hydride (26.2 mg, 656 umol, 60% purity, 2.00 eq) at 0° C. The mixture was stirred at 0° C. for 1 h. The mixture was adjusted to pH=5 with hydrochloric acid (1 M). Solid was precipitated from the mixture. The solid was collected by filtration and triturated with dimethyl formamide (2.00 mL). The mixture was filtered to give a filter cake which was lyophilized to afford (2-(2,6-dioxopiperidin-3-yl)-3-oxoisoindolin-5-yl)methyl (5-phenylpyridin-2-yl)carbamate 143. ¹H NMR (400 MHz, DMSO-d₆) δ=11.90 (br d, J=2.9 Hz, 1H), 10.97 (s, 1H), 8.65 (d, J=2.1 Hz, 1H), 8.57 (br d, J=9.0 Hz, 1H), 7.87 (d, J=9.0 Hz, 1H), 7.82 (s, 1H), 7.72 (d, J=7.5 Hz, 3H), 7.66 (s, 1H), 7.53-7.49 (m, 2H), 7.45 (d, J=7.4 Hz, 1H), 5.40 (s, 2H), 5.08 (br dd, J=4.9, 13.1 Hz, 1H), 4.51-4.43 (m, 1H), 4.38-4.28 (m, 1H), 2.93-2.84 (m, 1H), 2.60 (br s, 1H), 2.45-2.35 (m, 1H), 2.04-1.96 (m, 1H). MS (ESI) m/z 471.2 [M+H]⁺.

### Compound 344:

Step 1: To a solution of 3-chloro-5-methylaniline (1.00 g, 7.06 mmol, 1.00 eq) in Acetonitrile (5.00 mL) was added pyridine (2.79 g, 35.3 mmol, 2.85 mL, 5.00 eq) and phenyl carbonochloridate (1.66 g, 10.6 mmol, 1.33 mL, 1.50 eq) at 0° C. The mixture was stirred at 0° C. for 2 h. The mixture was filtered to give a filtrate which was concentrated and purified by reversed phase column chromatography. The desired fraction was collected and concentrated to give phenyl (3-chloro-5-methylphenyl)carbamate. ¹H NMR (400 MHz, DMSO-d₆) δ=10.38 (br s, 1H), 7.47-7.41 (m, 3H), 7.31-7.20 (m, 4H), 6.96 (s, 1H), 2.28 (s, 3H). MS (ESI) m/z 262.0 [M+H]⁺

Step 2: To a solution of 3-(6-(hydroxymethyl)-1-oxoisoindolin-2-yl)piperidine-2,6-dione VIII (80.0 mg, 292 umol, 1.00 eq) in dimethyl formamide (1.00 mL) was added phenyl (3-chloro-5-methylphenyl)carbamate (114 mg, 438 umol, 1.50 eq) and sodium hydride (23.3 mg, 583 umol, 60% purity, 2.00 eq) at 0° C. The mixture was stirred at 0° C. for 1 h. The mixture was adjusted to pH=5 with hydrochloric acid (1 M). The mixture was diluted with water/ethyl acetate (10.0 ml/10.0 ml). The organic layer was separated and concentrated to give a residue. The residue was purified by Prep-HPLC and the desired fraction was collected and lyophilized to afford (2-(2,6-dioxopiperidin-3-yl)-3-oxoisoindolin-5-yl)methyl (3-chloro-5-methylphenyl)carbamate 144. ¹H NMR (400 MHz, DMSO-d₆) δ=11.00 (br s, 1H), 9.97 (s, 1H), 7.80 (s, 1H), 7.73-7.62 (m, 2H), 7.41 (s, 1H), 7.22 (s, 1H), 6.90 (s, 1H), 5.28 (s, 2H), 5.13 (br dd, J=5.0, 13.3 Hz, 1H), 4.52-4.44 (m, 1H), 4.39-4.30 (m, 1H), 2.97-2.87 (m, 1H), 2.63 (br d, J=1.6 Hz, 1H), 2.41 (br dd, J=4.3, 12.9 Hz, 1H), 2.26 (s, 3H), 2.06-1.97 (m, 1H). MS (ESI) m/z 442.1 [M+H]⁺.

### Compound 345:

Step 1: A mixture of 2-fluoro-5-(trifluoromethoxy)aniline (500 mg, 2.56 mmol, 305 uL, 1.00 eq), phenyl carbonochloridate (441 mg, 2.82 mmol, 353 uL, 1.10 eq) and pyridine (608 mg, 7.69 mmol, 621 uL, 3.00 eq) in Acetonitrile (3.00 mL) was stirred at 25° C. for 2 h. The mixture was concentrated to give crude product. The crude product was purified by reversed phase column chromatography and the desired fraction was collected and lyophilized to give phenyl (2-fluoro-5-(trifluoromethoxy)phenyl)carbamate. MS (ESI) m/z 316.1 [M+H]⁺

Step 2: To a mixture of phenyl (2-fluoro-5-(trifluoromethoxy)phenyl)carbamate (126 mg, 401 umol, 1.10 eq) and 3-(6-(hydroxymethyl)-1-oxoisoindolin-2-yl)piperidine-2,6-dione VIII (100 mg, 365 umol, 1.00 eq) in dimethyformamide (2.00 mL) was added sodium hydride (29.2 mg, 729 umol, 60% purity, 2.00 eq) at 0° C. The mixture was stirred at 25° C. for 1 h. The mixture was quenched with 1 M hydrochloric acid and filtered. The filtrate was purified by prep-HPLC and the desired fraction was collected and lyophilized to afford (2-(2,6-dioxopiperidin-3-yl)-3-oxoisoindolin-5-yl)methyl(2-fluoro-5-(trifluoromethoxy) phenyl)carbamate 145. ¹H NMR (400 MHz, DMSO-d₆) δ=11.02 (s, 1H), 9.94 (s, 1H), 7.88-7.80 (m, 2H), 7.72-7.62 (m, 2H), 7.38 (dd, J=9.2, 10.4 Hz, 1H), 7.18-7.10 (m, 1H), 5.32 (s, 2H), 5.14 (dd, J=5.2, 13.4 Hz, 1H), 4.52-4.44 (m, 1H), 4.38-4.30 (m, 1H), 2.98-2.86 (m, 1H), 2.68-2.58 (m, 1H), 2.44-2.32 (m, 1H), 2.06-1.98 (m, 1H). MS (ESI) m/z 496.1 [M+H]⁺

### Compound 346:

Step 1: To a solution of 3-fluoro-5-methylaniline (1.00 g, 7.99 mmol, 1.00 eq) in Acetonitrile (5.00 mL) was added pyridine (3.16 g, 39.9 mmol, 3.22 mL, 5.00 eq) and phenyl carbonochloridate (1.88 g, 12.0 mmol, 1.50 mL, 1.50 eq). The mixture was stirred at 25° C. for 1 h. The mixture was filtered to give a filtrate which was purified by reversed phase column chromatography. The desired fraction was collected and concentrated to give phenyl (3-fluoro-5-methylphenyl)carbamate. ¹H NMR (400 MHz, DMSO-d₆) δ=10.39 (br s, 1H), 7.50-7.38 (m, 3H), 7.29-7.21 (m, 3H), 7.12 (s, 1H), 6.72 (br d, J=9.3 Hz, 1H), 2.29 (s, 3H). MS (ESI) m/z 246.0 [M+H]⁺

Step 2: To a solution of 3-(6-(hydroxymethyl)-1-oxoisoindolin-2-yl)piperidine-2,6-dione VIII (90.0 mg, 328 umol, 1.00 eq) in dimethyl formamide (2.00 mL) was added phenyl (3-fluoro-5-methylphenyl)carbamate (121 mg, 492 umol, 1.50 eq) and sodium hydride (26.2 mg, 656 umol, 60% purity, 2.00 eq) at 0° C. The mixture was stirred at 0° C. for 1 h. The mixture was adjusted to pH=5 with hydrochloric acid (1 M). The mixture was diluted with water/ethyl acetate (20.0 ml/20.0 ml). The organic layer was collected and concentrated to give a residue. The residue was purified by Prep-HPLC and the desired fraction was collected and lyophilized to afford (2-(2,6-dioxopiperidin-3-yl)-3-oxoisoindolin-5-yl)methyl (3-fluoro-5-methylphenyl) carbamate 146. ¹H NMR (400 MHz, DMSO-d₆) δ=11.00 (br s, 1H), 9.97 (s, 1H), 7.80 (s, 1H), 7.71-7.66 (m, 1H), 7.66-7.61 (m, 1H), 7.19 (br d, J=11.4 Hz, 1H), 7.07 (s, 1H), 6.65 (br d, J=9.7 Hz, 1H), 5.28 (s, 2H), 5.14 (dd, J=5.1, 13.2 Hz, 1H), 4.53-4.43 (m, 1H), 4.39-4.30 (m, 1H), 2.96-2.87 (m, 1H), 2.64-2.58 (m, 1H), 2.42 (br d, J=4.5 Hz, 1H), 2.27 (s, 3H), 2.02 (br d, J=5.3 Hz, 1H). MS (ESI) m/z 426.2 [M+H]⁺.

### Compound 347:

Step 1: To a solution of propanamide (9.00 g, 123 mmol, 1.00 eq) in toluene (400 mL) was added S-chloro chloromethanethioate (17.7 g, 135 mmol, 1.10 eq). The reaction mixture was allowed to stir at 100° C. for 5 h. The reaction mixture was concentrated under reduced pressure to give the crude product 5-ethyl-1,3,4-oxathiazol-2-one, which was used directly in next step without further purification. ¹H NMR (400 MHz, DMSO-d₆) δ=2.59 (q, J=7.6 Hz, 2H), 1.09 (t, J=7.6 Hz, 3H).

Step 2: To a solution of 5-ethyl-1,3,4-oxathiazol-2-one (8.50 g, 64.8 mmol, 1.00 eq) in 1,2-dichlorobenzene (100 mL) was added ethyl prop-2-ynoate (25.4 g, 259 mmol, 25.4 mL, 4.00 eq), the reaction mixture was allowed to stir at 140° C. for 48 h. The reaction mixture was diluted with tert-butyl methyl ether (500 mL) and washed with saturated aqueous sodium bicarbonate (3×200 mL), the organic phase was washed with brine (200 mL), dried over sodium sulfate, filtered and concentrated under reduced pressure. The residue was purified by column chromatography to afford a mixture of ethyl 3-ethylisothiazole-5-carboxylate/ethyl 3-ethylisothiazole-4-carboxylate (1/1), which was further to afford ethyl 3-ethylisothiazole-4-carboxylate. ¹H NMR (400 MHz, DMSO-d₆) δ=9.61 (s, 1H), 4.28 (q, J=7.2 Hz, 2H), 3.04 (q, J=7.6 Hz, 2H), 1.31 (t, J=7.2 Hz, 3H), 1.23 (t, J=7.6 Hz, 3H).

Step 3: To a solution of ethyl 3-ethylisothiazole-4-carboxylate (100 mg, 540 umol, 1.00 eq) in methanol (1.00 mL) was added a solution of lithium hydroxide monohydrate (113 mg, 2.70 mmol, 5.00 eq) in water (0.500 mL). The reaction mixture was stirred at 25° C. for 1 h. The reaction was concentrated under reduced pressure. The residue was diluted with water (5.00 mL) and adjusted pH=2-3 with 1 mol/L hydrochloric acid solution. The precipitate was formed and filtered to afford 3-ethylisothiazole-4-carboxylic acid, which was used for next step directly without further purification. ¹H NMR (400 MHz, DMSO-d₆) δ=13.21-12.77 (m, 1H), 9.56 (s, 1H), 3.05 (q, J=7.6 Hz, 2H), 1.23 (t, J=7.6 Hz, 3H).

Step 4: To a solution of 3-ethylisothiazole-4-carboxylic acid (40.0 mg, 254 umol, 1.00 eq) in dioxane (2.00 mL) was added triethylamine (77.2 mg, 763 umol, 106 uL, 3.00 eq) and diphenylphosphoryl azide (105 mg, 382 umol, 82.7 uL, 1.50 eq), the mixture was stirred at 25° C. for 1 h, then 3-[6-(hydroxymethyl)-1-oxo-isoindolin-2-yl]piperidine-2,6-dione (69.8 mg, 254 umol, 1.00 eq) was added. The reaction mixture was allowed to stir at 100° C. for 2 h under nitrogen. The reaction mixture was concentrated under reduced pressure and diluted with water (50.0 mL), extracted with ethyl acetate (3×30.0 mL), the organic phase was washed with brine (30.0 mL), dried over sodium sulfate, filtered and concentrated. The residue was purified by column chromatography to give crude product, which was purified by prep-HPLC to afford 2-(2,6-Dioxopiperidin-3-yl)-3-oxoisoindolin-5-yl)methyl (3-ethylisothiazol-4-yl)carbamate 147. ¹H NMR (400 MHz, DMSO-d₆) δ=11.00 (s, 1H), 9.73-9.45 (m, 1H), 8.76 (s, 1H), 7.81 (s, 1H), 7.73-7.59 (m, 2H), 5.30 (s, 2H), 5.13 (dd, J=5.2, 13.2 Hz, 1H), 4.53-4.30 (m, 2H), 2.98-2.85 (m, 1H), 2.74 (q, J=7.6 Hz, 2H), 2.61 (br dd, J=2.0, 15.6 Hz, 1H), 2.41 (br dd, J=4.8, 13.2 Hz, 1H), 2.07-1.96 (m, 1H), 1.19 (t, J=7.6 Hz, 3H). MS (ESI) m/z 429.0 [M+H]⁺

### Compound 348:

Step 1: To a mixture of 2-fluoro-4-(trifluoromethoxy)aniline (220 mg, 1.13 mmol, 1.00 eq) and phenyl carbonochloridate (265 mg, 1.69 mmol, 212 uL, 1.50 eq) in Acetonitrile (5.00 mL) was added pyridine (268 mg, 3.38 mmol, 273 uL, 3.00 eq) dropwise. The mixture was stirred at 25° C. for 1 h. The mixture was concentrated to give crude product. The crude product was purified by reversed-phase HPLC. The desired fraction was collected and lyophilized to give phenyl (2-fluoro-4-(trifluoromethoxy) phenyl)carbamate

Step 2: To a mixture of phenyl(2-fluoro-4-(trifluoromethoxy)phenyl)carbamate (110 mg, 350 umol, 1.20 eq) and 3-(6-(hydroxymethyl)-1-oxoisoindolin-2-yl)piperidine-2,6-dione VIII (80.0 mg, 292 umol, 1.00 eq) in dimethyformamide (2.00 mL) was added sodium hydride (17.5 mg, 438 umol, 60% purity, 1.50 eq) in portions. The mixture was stirred at 25° C. for 1 h. The mixture was quenched with 1 M hydrochloric acid and filtered. The filtrate was purified by Prep-HPLC and lyophilized to afford (2-(2,6-dioxopiperidin-3-yl)-3-oxoisoindolin-5-yl)methyl(2-fluoro-4-(trifluoromethoxy)phenyl)carbamate 148. ¹H NMR (400 MHz, DMSO-d₆) δ=11.00 (s, 1H), 9.74 (br s, 1H), 7.84-7.74 (m, 2H), 7.70-7.61 (m, 2H), 7.45 (dd, J=2.3, 10.9 Hz, 1H), 7.23 (br d, J=8.9 Hz, 1H), 5.29 (s, 2H), 5.13 (dd, J=5.1, 13.2 Hz, 1H), 4.51-4.43 (m, 1H), 4.38-4.30 (m, 1H), 2.97-2.86 (m, 1H), 2.60 (br d, J=18.2 Hz, 1H), 2.40 (dt, J=9.0, 13.2 Hz, 1H), 2.06-1.96 (m, 1H). MS (ESI) m/z 496.1 [M+H]⁺

### Compound 349:

Step 1: To a solution of 2-phenylpyridin-4-amine (500 mg, 2.94 mmol, 1.00 eq) and pyridine (1.16 g, 14.7 mmol, 1.19 mL, 5.00 eq) in Acetonitrile (5.00 mL) was added phenyl carbonochloridate (689 mg, 4.41 mmol, 552 uL, 1.50 eq) dropwise at 0° C. The mixture was stirred at 20° C. for 12 h. The mixture was concentrated to give crude product, which was purified by reversed-phase HPLC to give phenyl (2-phenylpyridin-4-yl)carbamate. MS (ESI) m/z 291.0 [M+H]⁺

Step 2: To a solution of 3-(6-(hydroxymethyl)-1-oxoisoindolin-2-yl)piperidine-2,6-dione VIII (80.0 mg, 292 umol, 1.00 eq) and phenyl (2-phenylpyridin-4-yl)carbamate (110 mg, 379 umol, 1.30 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched with 1 M hydrochloric acid (1.00 mL) and the mixture was purified by Prep-HPLC to afford (2-(2,6-dioxopiperidin-3-yl)-3-oxoisoindolin-5-yl)methyl(2-phenylpyridin-4-yl)carbamate 149. ¹H NMR (400 MHz, DMSO-d₆) δ=11.00 (s, 1H), 10.38 (s, 1H), 8.49 (d, J=5.5 Hz, 1H), 8.01-7.92 (m, 3H), 7.84 (s, 1H), 7.75-7.70 (m, 1H), 7.68-7.64 (m, 1H), 7.53-7.48 (m, 2H), 7.47-7.38 (m, 2H), 5.34 (s, 2H), 5.14 (dd, J=5.1, 13.4 Hz, 1H), 4.53-4.44 (m, 1H), 4.41-4.30 (m, 1H), 2.96-2.89 (m, 1H), 2.63 (br d, J=2.3 Hz, 1H), 2.44-2.35 (m, 1H), 2.07-1.97 (m, 1H). MS (ESI) m/z 471.2 [M+H]⁺

### Compound 350:

Step 1: A mixture of 2-methyl-5-nitrobenzoic acid (20.0 g, 110 mmol, 1.00 eq) and 1,3-dichloro-5,5-dimethylimidazolidine-2,4-dione (21.8 g, 110 mmol, 1.00 eq) in sulfuric acid (20.0 mL) was stirred at 80° C. for 10 h. The reaction mixture was poured into ice water (about 300 mL), after stirring, the precipitated solid was collected by filtration and washed with water to give 3-chloro-2-methyl-5-nitrobenzoic acid, which was used for next step without further purification. ¹H NMR (400 MHz, CDCl₃) δ=8.67 (d, J=2.4 Hz, 1H), 8.36 (d, J=2.4 Hz, 1H), 2.72 (s, 3H).

Step 2: To a solution of 3-chloro-2-methyl-5-nitrobenzoic acid (24.0 g, 111 mmol, 1.00 eq) in tetrahydrofuran (200 mL) was added borane dimethyl sulfide complex (10.0 M, 22.3 mL, 2.00 eq) at 0° C. Then the mixture was stirred at 25° C. for 10 h. The reaction mixture was quenched by addition water (50.0 mL), and then extracted with ethyl acetate (3×200 mL). The combined organic layers were washed with brine, dried over sodium sulfate, filtered and concentrated under reduced pressure to give 3-chloro-2-methyl-5-nitrophenyl)methanol. ¹H NMR (400 MHz, CDCl₃) δ=8.16 (d, J=2.2 Hz, 1H), 8.09 (d, J=2.2 Hz, 1H), 4.72 (s, 2H), 2.35 (s, 3H).

Step 3: To a solution of (3-chloro-2-methyl-5-nitrophenyl)methanol (23.0 g, 114 mmol, 1.00 eq) in dichloromethane (200 mL) was added thionyl chloride (67.9 g, 570 mmol, 41.4 mL, 5.00 eq). Then the mixture was stirred at 25° C. for 10 h. The reaction mixture was poured into ice water and then extracted with dichloromethane. The combined organic layers were washed with saturated sodium bicarbonate and brin), dried over magnesium sulfate, filtered and concentrated under reduced pressure to give 1-chloro-3-(chloromethyl)-2-methyl-5-nitrobenzene. ¹H NMR (400 MHz, CDCl₃) δ=8.24 (d, J=2.4 Hz, 1H), 8.14 (d, J=2.4 Hz, 1H), 4.67 (s, 2H), 2.56 (s, 3H).

Step 4: To a solution of sodium hydride (1.00 g, 25.0 mmol, 60% purity, 1.10 eq) in N,N-dimethylformamide (50.0 mL) was added diethyl 2-acetamidomalonate (5.92 g, 27.3 mmol, 1.20 eq) at 0° C. After 5 mins, 1-chloro-3-(chloromethyl)-2-methyl-5-nitrobenzene (5.00 g, 22.7 mmol, 1.00 eq) was added. Then the mixture was stirred at 25° C. for 10 h. The reaction mixture was quenched by addition water, then extracted with ethyl acetate. The combined organic layers were washed with brine, dried over sodium sulfate, filtered and concentrated under reduced pressure to give diethyl 2-acetamido-2-(3-chloro-2-methyl-5-nitrobenzyl)malonate. ¹H NMR (400 MHz, CDCl₃) δ=8.13 (d, J=2.4 Hz, 1H), 7.77 (d, J=2.4 Hz, 1H), 6.60 (s, 1H), 4.33-4.26 (m, 4H), 3.85 (s, 2H), 2.35 (s, 3H), 2.04 (s, 3H), 1.33-1.30 (m, 6H).

Step 5: A mixture of diethyl 2-acetamido-2-(3-chloro-2-methyl-5-nitrobenzyl)malonate (9.20 g, 23.0 mmol, 1.00 eq) in hydrochloric acid (24.3 g, 240 mmol, 40.0 mL, 36% purity, 10.5 eq) was stirred at 110° C. for 10 h. The reaction mixture was filtered and the filtrate was lyophilized to afford 2-amino-3-(3-chloro-2-methyl-5-nitrophenyl)propanoic acid. ¹H NMR (400 MHz, DMSO-d₆) δ=8.85 (br s, 2H), 8.62-8.54 (m, 1H), 8.22 (d, J=2.4 Hz, 1H), 8.16 (d, J=2.4 Hz, 1H), 4.13 (brt, J=7.2 Hz, 1H), 3.38-3.35 (m, 2H), 2.47 (s, 3H).

Step 6: To a solution of 2-amino-3-(3-chloro-2-methyl-5-nitrophenyl)propanoic acid (3.00 g, 11.6 mmol, 1.00 eq) in tetrahydrofuran (30.0 mL) was added borane dimethyl sulfide complex (10.0 M, 3.48 mL, 3.00 eq). Then the mixture was stirred at 70° C. for 10 h. The reaction mixture was quenched by addition methanol at 0° C., then concentrated under reduced pressure to give 2-amino-3-(3-chloro-2-methyl-5-nitrophenyl)propan-1-ol. MS (ESI) m/z 245.1 [M+H]⁺.

Step 7: To a solution of 2-amino-3-(3-chloro-2-methyl-5-nitrophenyl)propan-1-ol (3.00 g, 12.3 mmol, 1.00 eq) and triethylamine (1.49 g, 14.7 mmol, 2.05 mL, 1.20 eq) in tetrahydrofuran (30.0 mL) was added 2-chloroacetyl chloride (1.66 g, 14.7 mmol, 1.17 mL, 1.20 eq) at 0° C. Then the mixture was stirred at 25° C. for 1 h, quenched by addition water, and then extracted with ethyl acetate. The combined organic layers were washed with brine, dried over sodium sulfate, filtered and concentrated under reduced pressure to give a residue. The residue was purified by column chromatography to afford 2-chloro-N-(1-(3-chloro-2-methyl-5-nitrophenyl)-3-hydroxypropan-2-yl)acetamide. ¹H NMR (400 MHz, CDCl₃) δ=8.15 (d, J=2.4 Hz, 1H), 7.99 (d, J=2.4 Hz, 1H), 7.05 (brd, J=8.0 Hz, 1H), 4.26-4.15 (m, 1H), 4.04 (d, J=5.6 Hz, 2H), 3.78-3.70 (m, 1H), 3.69-3.62 (m, 1H), 3.10 (dd, J=7.6, 9.6 Hz, 2H), 2.56 (s, 3H).

Step 8: To a solution of 2-chloro-N-(1-(3-chloro-2-methyl-5-nitrophenyl)-3-hydroxypropan-2-yl)acetamide (1.20 g, 3.74 mmol, 1.00 eq) in t-butyl alcohol (3.00 mL) was added potassium tert-butoxide (839 mg, 7.47 mmol, 2.00 eq). Then the mixture was stirred at 100° C. for 0.5 h. The reaction mixture was concentrated under reduced pressure to remove t-nutyl alcohol. The residue was diluted with water and extracted with ethyl acetate. The combined organic layers were washed with brine, dried over sodium sulfate, filtered and concentrated under reduced pressure to give a residue. The residue was purified by column chromatography to afford 5-(3-chloro-2-methyl-5-nitrobenzyl)morpholin-3-one. ¹H NMR (400 MHz, DMSO-d₆) δ=8.17 (d, J=2.4 Hz, 1H), 8.13 (s, 1H), 8.02 (d, J=2.4 Hz, 1H), 4.00 (d, J=5.0 Hz, 2H), 3.63-3.54 (m, 2H), 3.50-3.42 (m, 1H), 3.08-2.97 (m, 2H), 2.44 (s, 3H).

Step 9: To a solution of 5-(3-chloro-2-methyl-5-nitrobenzyl)morpholin-3-one (240 mg, 843 umol, 1.00 eq) in tetrahydrofuran (3.00 mL) was added borane dimethyl sulfide complex (10.0 M, 253 uL, 3.00 eq). Then the mixture was stirred at 70° C. for 10 h. The reaction mixture was quenched by addition methanol, then concentrated under reduced pressure to give 3-(3-chloro-2-methyl-5-nitrobenzyl)morpholine. MS (ESI) m/z 271.2 [M+H]⁺.

Step 10: To a solution of 3-(3-chloro-2-methyl-5-nitrobenzyl)morpholine (0.150 g, 554 umol, 1.00 eq) and formaldehyde (981 mg, 12.1 mmol, 900 uL, 37% purity, 21.8 eq) in methanol (2.00 mL) was added acetic acid (66.6 mg, 1.11 mmol, 63.4 uL, 2.00 eq). After 0.5 h, sodium cyanoborohydride (174 mg, 2.77 mmol, 5.00 eq) was added and the mixture was stirred at 25° C. for 10 h. The reaction mixture was quenched by addition water, and then extracted with dichloromethane. The combined organic layers were washed with brine, dried over sodium sulfate, filtered and concentrated under reduced pressure to give a residue. The crude product was purified by reversed-phase HPLC to afford 3-(3-chloro-2-methyl-5-nitrobenzyl)-4-methylmorpholine. ¹H NMR (400 MHz, CDCl₃) δ=8.08 (d, J=2.4 Hz, 1H), 7.87 (d, J=2.4 Hz, 1H), 3.78 (br dd, J=3.4, 5.2 Hz, 2H), 3.50-3.37 (m, 2H), 3.21 (br dd, J=4.2, 13.8 Hz, 1H), 2.97-2.79 (m, 3H), 2.71-2.64 (m, 1H), 2.56 (s, 3H), 2.43 (s, 3H).

Step 11: A mixture of 3-(3-chloro-2-methyl-5-nitrobenzyl)-4-methylmorpholine (0.140 g, 492 umol, 1.00 eq), ferrous powder (137 mg, 2.46 mmol, 5.00 eq) and ammonium chloride (26.3 mg, 492 umol, 1.00 eq) in ethyl alcohol (2.00 mL) and water (1.00 mL) was stirred at 60° C. for 10 h. The reaction mixture was concentrated under reduced pressure to remove ethyl alcohol. The residue was diluted with water and extracted with ethyl acetate. The combined organic layers were washed with brine, dried over sodium sulfate, filtered and concentrated under reduced pressure to give 3-chloro-4-methyl-5-((4-methylmorpholin-3-yl)methyl)aniline. ¹H NMR (400 MHz, CDCl₃) δ=6.55 (d, J=2.4 Hz, 1H), 6.29 (d, J=2.4 Hz, 1H), 3.74-3.58 (m, 3H), 3.46 (br dd, J=2.0, 11.4 Hz, 1H), 3.27-3.20 (m, 1H), 3.06-3.01 (m, 1H), 2.69 (td, J=3.2, 11.8 Hz, 1H), 2.39 (s, 3H), 2.34 (brt, J=3.8 Hz, 2H), 2.18 (s, 3H).

Step 12: To a solution of 3-chloro-4-methyl-5-((4-methylmorpholin-3-yl)methyl)aniline (0.100 g, 393 umol, 1.00 eq) and potassium carbonate (109 mg, 785 umol, 2.00 eq) in acetone (2.00 mL) was added phenyl carbonochloridate (73.8 mg, 471 umol, 59.0 uL, 1.20 eq) at 0° C. Then the mixture was stirred at 25° C. for 10 h. The reaction mixture was diluted with water and extracted with ethyl acetate. The combined organic layers were washed with brine, dried over sodium sulfate, filtered and concentrated under reduced pressure to give phenyl (3-chloro-4-methyl-5-((4-methylmorpholin-3-yl) methyl)phenyl)carbamate. MS (ESI) m/z 375.2 [M+H]⁺.

Step 13: To a solution of 3-(6-(hydroxymethyl)-1-oxoisoindolin-2-yl)piperidine-2,6-dione VIII (40.0 mg, 146 umol, 1.00 eq) and phenyl (3-chloro-4-methyl-5-((4-methylmorpholin-3-yl)methyl)phenyl)carbamate (60.0 mg, 160 umol, 1.10 eq) in dimethyl formamide (2.00 mL) was added sodium hydride (60.0%, dispersion in paraffin liquid) (11.5 mg, 288 umol, 60.0% purity, 1.97 eq) in portions at 0° C. The mixture was stirred at 25° C. for 1 h. The mixture was adjusted to pH=7 with hydrochloric acid (1 M) and filtered to give a solution. The solution was purified by Prep-HPLC. The desired fraction was collected and lyophilized to give a solid, which was further purified by Prep-HPLC. The desired fraction was collected and lyophilized to afford (2-(2,6-dioxopiperidin-3-yl)-3-oxoisoindolin-5-yl) methyl(3-chloro-4-methyl-5-((4-methylmorpholin-3-yl)methyl)phenyl)carbamate 150. ¹H NMR (400 MHz, DMSO-d₆) δ=11.71 (br s, 1H), 11.00 (s, 1H), 9.95 (br s, 1H), 7.78 (s, 1H), 7.72-7.66 (m, 1H), 7.65-7.60 (m, 1H), 7.50 (s, 1H), 7.33-7.24 (m, 1H), 5.27 (s, 2H), 5.12 (dd, J=5.1, 13.2 Hz, 1H), 4.51-4.43 (m, 1H), 4.37-4.29 (m, 1H), 3.93 (br d, J=12.3 Hz, 1H), 3.88-3.78 (m, 1H), 3.68-3.58 (m, 1H), 3.56-3.48 (m, 1H), 3.47-3.38 (m, 2H), 3.29-3.13 (m, 1H), 3.12-2.92 (m, 1H), 2.92-2.89 (m, 3H), 2.89-2.84 (m, 1H), 2.60 (br d, J=17.3 Hz, 1H), 2.48-2.32 (m, 2H), 2.32-2.23 (m, 3H), 2.05-1.96 (m, 1H). MS (ESI) m/z 555.2 [M+H]⁺

### Compound 351:

Step 1: To a solution of propanamide (9.00 g, 123 mmol, 1.00 eq) in toluene (400 mL) was added chlorocarbonylsulfenylchloride (17.7 g, 135 mmol, 1.10 eq). The reaction mixture was allowed to stir at 100° C. for 5 h. The reaction mixture was concentrated under reduced pressure to give the crude product 5-ethyl-1,3,4-oxathiazol-2-one, which was used directly in next step without further purification. ¹H NMR (400 MHz, DMSO-d₆) δ=2.59 (q, J=7.6 Hz, 2H), 1.09 (t, J=7.6 Hz, 3H).

Step 2: To a solution of 5-ethyl-1,3,4-oxathiazol-2-one (8.50 g, 64.8 mmol, 1.00 eq) in 1,2-dichlorobenzene (100 mL) was added ethyl prop-2-ynoate (25.4 g, 259 mmol, 25.4 mL, 4.00 eq), the reaction mixture was allowed to stir at 140° C. for 48 h. The reaction mixture was diluted with tert-butyl methyl ether (500 mL) and washed with saturated aqueous sodium bicarbonate (3×200 mL), the organic phase was washed with brine, dried over sodium sulfate, filtered and concentrated under reduced pressure. The residue was purified by column chromatography to afford a mixture of ethyl 3-ethylisothiazole-5-carboxylate/ethyl 3-ethylisothiazole-4-carboxylate (1/1). ¹H NMR (400 MHz, DMSO-d₆) δ=9.55 (s, 1H), 7.72 (s, 1H), 4.35-4.16 (m, 2H), 2.98 (q, J=7.6 Hz, 1H), 2.77 (q, J=7.6 Hz, 1H), 1.28-1.13 (m, 6H). MS (ESI) m/z 186.0 [M+H]⁺

Step 3: To a mixture of ethyl 3-ethylisothiazole-5-carboxylate and ethyl 3-ethylisothiazole-4-carboxylate (1.00 g, 5.40 mmol, 1.00 eq) in tetrahydrofuran (9 mL) was added a solution of lithium hydroxide monohydrate (453 mg, 10.8 mmol, 2.00 eq) in water (3 mL), the reaction mixture was stirred at 25° C. for 1 h. Tetrahydrofuran was removed under reduced pressure. The residue was diluted with water and adjusted pH=2-3 with 1 mol/L hydrochloric acid solution. The precipitate was formed and filtered to collect the solid, which was dried in vacuum to afford a mixture of 3-ethylisothiazole-5-carboxylic acid/3-ethylisothiazole-4-carboxylic acid (1/1), which was used for next step directly without further purification. ¹H NMR (400 MHz, DMSO-d₆) δ=9.55 (s, 1H), 7.69 (s, 1H), 3.04 (q, J=7.6 Hz, 3H), 2.81 (q, J=7.6 Hz, 3H), 1.23 (td, J=7.6, 9.2 Hz, 6H).

Step 4: To a mixture of 3-ethylisothiazole-5-carboxylic acid/3-ethylisothiazole-4-carboxylic acid (100 mg, 636 umol, 1.00 eq) in dioxane (6 mL) was added triethylamine (193 mg, 1.91 mmol, 3.00 eq) and diphenylphosphoryl azide (262 mg, 954 umol, 206 uL, 1.50 eq), the mixture was stirred at 25° C. for 1 h, then 3-(6-(hydroxymethyl)-1-oxoisoindolin-2-yl)piperidine-2,6-dione VIII (174 mg, 636 umol, 1.00 eq) was added. The reaction mixture was allowed to stir at 110° C. for 2 h under nitrogen. The reaction mixture was concentrated under reduced pressure and diluted with water, extracted with ethyl acetate, the organic phase was washed with brine, dried over sodium sulfate, filtered and concentrated. The residue was purified by column chromatography to give crude product, which was purified by prep-HPLC to afford (2-(2,6-dioxopiperidin-3-yl)-3-oxoisoindolin-5-yl)methyl (3-methylisothiazol-5-yl)carbamate 151. ¹H NMR (400 MHz, DMSO-d₆) δ=11.54 (brd, J=1.2 Hz, 1H), 11.00 (br s, 1H), 7.80 (s, 1H), 7.73-7.60 (m, 2H), 6.60 (s, 1H), 5.36 (s, 2H), 5.13 (dd, J=5.2, 13.2 Hz, 1H), 4.55-4.28 (m, 2H), 3.00-2.85 (m, 1H), 2.68-2.58 (m, 3H), 2.44-2.33 (m, 1H), 2.07-1.95 (m, 1H), 1.17 (t, J=7.6 Hz, 3H). MS (ESI) m/z 429.0 [M+H]⁺

### Compound 352:

Step 1: To a solution of 2-chloro-5-nitropyridine (5.00 g, 31.5 mmol, 1.00 eq), potassium trifluoro(prop-1-en-2-yl) borate (7.00 g, 47.3 mmol, 1.50 eq) and potassium carbonate (13.1 g, 94.6 mmol, 3.00 eq) in dioxane (50.0 mL) and water (10.0 mL) was added [1,1′-bis(diphenylphosphino)ferrocene]dichloropalladium(II) (2.58 g, 3.15 mmol, 0.100 eq) then evacuated with vacuum and back filled with nitrogen 3 times. The mixture was stirred at 80° C. for 2 h. The mixture was diluted with water and extracted with ethyl acetate. The combined organic layer was washed with brine and dried over sodium sulfate, filtered and concentrated to give crude product, which was purified by reversed-phase HPLC to give 5-nitro-2-(prop-1-en-2-yl)pyridine. MS (ESI) m/z 165.1 [M+H]⁺

Step 2: To dichloromethane (20.0 mL) was added diethylzinc (1.00 M solution in toluene, 73.1 mL, 4.00 eq). The solution was cooled to −40° C. then diiodomethane (19.6 g, 73.1 mmol, 5.90 mL, 4.00 eq) in dichloromethane (10.0 mL) was added very slowly into the reaction mixture. The mixture was stirred at −40° C. for 30 min. Then trifluoroacetic acid (417 mg, 3.65 mmol, 271 uL, 0.200 eq) and N,N-dimethylacetamide (1.59 g, 18.3 mmol, 1.70 mL, 1.00 eq) in dichloromethane (5.00 mL) was added to and the mixture was stirred at −15° C. for another 0.5 h. Then 5-nitro-2-(prop-1-en-2-yl)pyridine (3.00 g, 18.3 mmol, 1.00 eq) in dichloromethane (10.0 mL) was added to at 0° C. The mixture was stirred at 20° C. for another 12 h, diluted with water and extracted with ethyl acetate. The combined organic layer was washed with brine and dried over sodium sulfate, filtered and concentrated to give crude product, which was purified by silica gel chromatography to give 2-(1-methylcyclopropyl)-5-nitropyridine. MS (ESI) m/z 179.0 [M+H]⁺

Step 3: To a solution of 2-(1-methylcyclopropyl)-5-nitropyridine (500 mg, 2.81 mmol, 1.00 eq) in tetrahydrofuran (10.0 mL) was added Pd/C (100 mg, 10% purity) and stirred at 20° C. for 1 h under hydrogen (15 Psi). The mixture was filtered and the filtrate was concentrated in vacuum. The residue was purified by reversed-phase HPLC to give 6-(1-methylcyclopropyl)pyridin-3-amine. MS (ESI) m/z 149.3 [M+H]⁺

Step 4: To a solution of 6-(1-methylcyclopropyl)pyridin-3-amine (40.0 mg, 269 umol, 1.00 eq) and pyridine (106 mg, 1.35 mmol, 108 uL, 5.00 eq) in Acetonitrile (1.00 mL) was added phenyl carbonochloridate (54.9 mg, 351 umol, 43.9 uL, 1.30 eq) at 0° C. The mixture was stirred at 20° C. for 2 h. The mixture was concentrated to give crude product and purified by reversed-phase HPLC to give phenyl (6-(1-methylcyclopropyl)pyridin-3-yl)carbamate. MS (ESI) m/z 269.1 [M+H]⁺

Step 5: To a solution of 3-(6-(hydroxymethyl)-1-oxoisoindolin-2-yl)piperidine-2,6-dione VIII (46.5 mg, 169 umol, 1.00 eq) and phenyl (6-(1-methylcyclopropyl)pyridin-3-yl)carbamate (50.0 mg, 186 umol, 1.10 eq) in dimethyl formamide (1.00 mL) was added sodium hydride (13.5 mg, 339 umol, 60% purity, 2.00 eq) at 0° C. The mixture was stirred at 20° C. for 1 h. The mixture was quenched by 1 M hydrochloric acid, purified by prep-HPLC (and lyophilized to give (2-(2,6-dioxopiperidin-3-yl)-3-oxoisoindolin-5-yl)methyl (6-(1-methylcyclopropyl)pyridin-3-yl)carbamate 152. ¹H NMR (400 MHz, DMSO-d₆) δ=11.00 (s, 1H), 10.51-10.25 (m, 1H), 8.64 (br d, J=2.9 Hz, 1H), 8.17-7.96 (m, 1H), 7.81 (s, 1H), 7.73-7.68 (m, 1H), 7.67-7.64 (m, 1H), 7.60 (br s, 1H), 5.32 (s, 2H), 5.13 (dd, J=5.0, 13.3 Hz, 1H), 4.52-4.45 (m, 1H), 4.38-4.31 (m, 1H), 2.96-2.87 (m, 1H), 2.61 (br d, J=17.8 Hz, 1H), 2.45-2.36 (m, 1H), 2.07-1.97 (m, 1H), 1.48 (s, 3H), 1.17 (br s, 2H), 0.91 (br d, J=2.8 Hz, 2H). MS (ESI) m/z 449.2 [M+H]⁺

### Compound 353:

Step 1: A mixture of 3-(tert-butyl)aniline (500 mg, 3.35 mmol, 1.00 eq), phenyl carbonochloridate (787 mg, 5.03 mmol, 629 uL, 1.50 eq) and pyridine (795 mg, 10.1 mmol, 811 uL, 3.00 eq) in Acetonitrile (5.00 mL) was stirred at 25° C. for 12 h. The mixture was concentrated to give crude product. The crude product was purified by reversed-phase HPLC. The desired fraction was collected and lyophilized to give phenyl (3-(tert-butyl)phenyl)carbamate. ¹H NMR (400 MHz, DMSO-d₆) δ=10.13 (br s, 1H), 7.59 (br s, 1H), 7.47-7.40 (m, 2H), 7.33 (br d, J=8.1 Hz, 1H), 7.29-7.19 (m, 4H), 7.09 (br d, J=7.6 Hz, 1H), 1.27 (s, 9H).

Step 2: To a mixture of phenyl (3-(tert-butyl)phenyl)carbamate (94.3 mg, 350 umol, 1.20 eq) and 3-(6-(hydroxymethyl)-1-oxoisoindolin-2-yl)piperidine-2,6-dione VIII (80.0 mg, 292 umol, 1.00 eq) in dimethyformamide (2.00 mL) was added sodium hydride (17.5 mg, 438 umol, 60% purity, 1.50 eq) in one portion at 0° C. The mixture was stirred at 20° C. for 1 h. The mixture was quenched with 1 M hydrochloric acid and filtered. The filtrate was purified by prep-HPLC and lyophilized to give (2-(2,6-dioxopiperidin-3-yl)-3-oxoisoindolin-5-yl)methyl (3-(tert-butyl)phenyl)carbamate 153. ¹H NMR (400 MHz, DMSO-d₆) δ=10.99 (s, 1H), 9.71 (br s, 1H), 7.79 (s, 1H), 7.71-7.60 (m, 2H), 7.51 (br s, 1H), 7.30 (br d, J=7.7 Hz, 1H), 7.19 (t, J=7.9 Hz, 1H), 7.03 (br d, J=7.6 Hz, 1H), 5.27 (s, 2H), 5.13 (br dd, J=5.0, 13.2 Hz, 1H), 4.51-4.42 (m, 1H), 4.39-4.29 (m, 1H), 2.98-2.84 (m, 1H), 2.60 (br d, J=17.1 Hz, 1H), 2.40 (dq, J=4.4, 13.2 Hz, 1H), 2.06-1.96 (m, 1H), 1.24 (s, 9H). MS (ESI) m/z 450.2 [M+H]⁺

### Compound 354:

Step 1: A mixture of 4-(trifluoromethoxy)aniline (400 mg, 2.26 mmol, 305 uL, 1.00 eq), phenyl carbonochloridate (389 mg, 2.48 mmol, 311 uL, 1.10 eq) and pyridine (536 mg, 6.77 mmol, 547 uL, 3.00 eq) in Acetonitrile (3.00 mL) was stirred at 25° C. for 2 h. The mixture was concentrated to give crude product, which was purified by reversed-phase HPLC. The desired fraction was collected and lyophilized to give phenyl (4-(trifluoromethoxy)phenyl)carbamate. MS (ESI) m/z 298.0 [M+H]⁺

Step 2: To a mixture of phenyl (4-(trifluoromethoxy)phenyl)carbamate (141 mg, 474 umol, 1.30 eq) and 3-(6-(hydroxymethyl)-1-oxoisoindolin-2-yl)piperidine-2,6-dione VIII (100 mg, 365 umol, 1.00 eq) in dimethyformamide (2.00 mL) was added sodium hydride (29.2 mg, 729 umol, 60% purity, 2.00 eq) in one portion at 0° C. The mixture was stirred at 25° C. for 1 h. The mixture was quenched with 1 M hydrochloric acid and filtered. The filtrate was purified by prep-HPLC and lyophilized to give (2-(2,6-dioxopiperidin-3-yl)-3-oxoisoindolin-5-yl)methyl (4-(trifluoromethoxy)phenyl)carbamate 154. ¹H NMR (400 MHz, DMSO-d₆) δ=11.02 (s, 1H), 10.04 (s, 1H), 7.82 (s, 1H), 7.72-7.68 (m, 1H), 7.66-7.64 (m, 1H), 7.58 (d, J=9.2 Hz, 2H), 7.32 (d, J=8.4 Hz, 2H), 5.28 (s, 2H), 5.14 (dd, J=5.2, 13.4 Hz, 1H), 4.48 (d, J=17.6 Hz, 1H), 4.36 (d, J=17.4 Hz, 1H), 2.98-2.86 (m, 1H), 2.66-2.56 (m, 1H), 2.48-2.36 (m, 1H), 2.08-1.98 (m, 1H). MS (ESI) m/z 478.1 [M+H]⁺

### Compound 355:

Step 1: To a solution of 4-phenylpyridin-2-amine (500 mg, 2.94 mmol, 1.00 eq) in Acetonitrile (10.0 mL) was added pyridine (1.16 g, 14.6 mmol, 1.19 mL, 5.00 eq) and phenyl carbonochloridate (597 mg, 3.82 mmol, 478 uL, 1.30 eq). The mixture was stirred at 25° C. for 0.5 h. The reaction mixture was concentrated under reduced pressure to give a residue. The crude product was triturated with water (10.0 ml) and filtered. The filter cake was washed with water and dried to give phenyl (4-phenylpyridin-2-yl)carbamate. ¹H NMR (400 MHz, DMSO-d₆) δ=10.86 (s, 1H), 8.40 (d, J=5.3 Hz, 1H), 8.13 (d, J=0.9 Hz, 1H), 7.77-7.70 (m, 2H), 7.53-7.49 (m, 3H), 7.47-7.43 (m, 3H), 7.30-7.22 (m, 3H).

Step 2: To a solution of 3-(6-(hydroxymethyl)-1-oxoisoindolin-2-yl)piperidine-2,6-dione VIII (80.0 mg, 291 umol, 1.00 eq) in dimethyl formamide (2.00 mL) was added phenyl (4-phenylpyridin-2-yl)carbamate (127 mg, 437 umol, 1.50 eq) and sodium hydride (23.3 mg, 583 umol, 60% purity, 2.00 eq) at 0° C. The mixture was stirred at 0° C. for 0.5 h. The reaction mixture was quenched with hydrochloric acid (1.00M, 1.00 ml) to give a solution. The solution was purified by prep-HPLC and lyophilized to give (2-(2,6-dioxopiperidin-3-yl)-3-oxoisoindolin-5-yl)methyl (4-phenylpyridin-2-yl)carbamate 155 d. ¹H NMR (400 MHz, DMSO-d₆) δ=11.07-10.92 (m, 2H), 8.37 (d, J=5.5 Hz, 1H), 8.07 (d, J=1.1 Hz, 1H), 7.85 (s, 1H), 7.78-7.63 (m, 4H), 7.61-7.47 (m, 4H), 5.37 (s, 2H), 5.13 (dd, J=5.1, 13.3 Hz, 1H), 4.52-4.43 (m, 1H), 4.40-4.30 (m, 1H), 2.98-2.85 (m, 1H), 2.65-2.57 (m, 1H), 2.41 (br dd, J=4.4, 13.1 Hz, 1H), 2.06-1.97 (m, 1H). MS (ESI) m/z 471.2 [M+H]⁺.

### Compound 356:

Step 1: To a mixture of 2-chloro-5-nitropyridine (652 mg, 4.11 mmol, 1.00 eq) and (S)-2-methylpyrrolidine hydrochloride (500 mg, 4.11 mmol, 1.00 eq, HCl) in dimethylformamide (2.00 mL) was added potassium carbonate in one portion at 25° C. The mixture was stirred at 60° C. for 2 h. The reaction mixture was diluted with water and extracted with ethyl acetate. The organic phase was separated, washed with brine, dried over anhydrous sodium sulfate, filtered and concentrated under reduced pressure to give (S)-2-(2-methylpyrrolidin-1-yl)-5-nitropyridine. MS (ESI) m/z 208.2 [M+H]⁺.

Step 2: To a solution of (S)-2-(2-methylpyrrolidin-1-yl)-5-nitropyridine (620 mg, 2.99 mmol, 1.00 eq) in methanol (6.00 mL) and water (3.00 mL) was added iron powder (835 mg, 15.0 mmol, 5.00 eq) and ammonium chloride (800 mg, 15.0 mmol, 5.00 eq) in one portion at 25° C. and stirred at 80° C. for 2 h. The mixture was filtered to give a filtrate, which was concentrated under reduced pressure to give a residue. The residue was diluted with water and extracted with ethyl acetate. The organic phase was separated, washed with brine, dried over anhydrous sodium sulfate, filtered and concentrated under reduced pressure to give (S)-6-(2-methylpyrrolidin-1-yl)pyridin-3-amine.

Step 3: To a solution of (S)-6-(2-methylpyrrolidin-1-yl)pyridin-3-amine (500 mg, 2.82 mmol, 1.00 eq) and pyridine (669 mg, 8.46 mmol, 683 uL, 3.00 eq) in Acetonitrile (2.00 mL) was added phenyl carbonochloridate (486 mg, 3.10 mmol, 389 uL, 1.10 eq) at 25° C. The reaction mixture was stirred at 25° C. for 2 h. The reaction mixture was concentrated to give a residue, which was purified by reversed phase and lyophilized to give (S)-phenyl (6-(2-methylpyrrolidin-1-yl)pyridin-3-yl)carbamate. MS (ESI) m/z 298.2 [M+H]⁺.

Step 4: To a mixture of 3-(6-(hydroxymethyl)-1-oxoisoindolin-2-yl)piperidine-2,6-dione VIII (80.0 mg, 292 umol, 1.00 eq) and (S)-phenyl (6-(2-methylpyrrolidin-1-yl)pyridin-3-yl)carbamate (113 mg, 379 umol, 1.30 eq) in dimethyl formamide (2.00 mL) was added sodium hydride (23.3 mg, 583 umol, 60% purity, 2.00 eq) at 0° C. The reaction mixture was stirred at 25° C. for 2 h. The reaction mixture was added hydrochloric acid (1 M, 2.00 mL) and filtered to give a filter cake. The filter cake was purified by prep-HPLC and lyophilized to give (2-(2,6-dioxopiperidin-3-yl)-3-oxoisoindolin-5-yl)methyl (6-((S)-2-methylpyrrolidin-1-yl)pyridin-3-yl)carbamate 156. ¹H NMR (400 MHz, DMSO-d₆) δ=13.74 (br s, 1H), 11.00 (s, 1H), 10.16 (br s, 1H), 8.20 (br s, 1H), 7.98 (br d, J=9.6 Hz, 1H), 7.80 (s, 1H), 7.72-7.62 (m, 2H), 7.18 (br d, J=9.8 Hz, 1H), 5.30 (s, 2H), 5.14 (dd, J=5.0, 13.4 Hz, 1H), 4.48 (s, 1H), 4.36 (br d, J=17.6 Hz, 1H), 4.32 (br s, 1H), 3.68 (brt, J=8.8 Hz, 1H), 3.50-3.36 (m, 1H), 3.04-2.80 (m, 1H), 2.62 (br d, J=17.4 Hz, 1H), 2.48-2.34 (m, 1H), 2.20-1.98 (m, 4H), 1.84-1.72 (m, 1H), 1.18 (d, J=6.4 Hz, 3H). MS (ESI) m/z 478.2 [M+H]⁺.

### Compound 357

Step 1: To a solution of 2-chloro-5-nitropyridine (652 mg, 4.11 mmol, 1.00 eq) and (R)-2-methylpyrrolidine hydrochloride (500 mg, 4.11 mmol, 1.00 eq, HCl) in dimethyl formamide (3.00 mL) was added potassium carbonate (1.70 g, 12.3 mmol, 3.00 eq) and stirred at 60° C. for 2 h. The mixture was diluted with water (50.0 mL) and extracted with ethyl acetate (3×30.0 mL). The combined organic layer was washed with brine (20.0 mL) and dried over anhydrous sodium sulfate, filtered and concentrated to give (R)-2-(2-methylpyrrolidin-1-yl)-5-nitropyridine. ¹H NMR (400 MHz, DMSO-d₆) δ=8.96 (d, J=2.7 Hz, 1H), 8.18 (dd, J=2.8, 9.5 Hz, 1H), 6.57 (br s, 1H), 4.51-4.13 (m, 1H), 3.61 (br s, 1H), 3.50-3.39 (m, 1H), 2.16-1.94 (m, 3H), 1.73 (br s, 1H), 1.19 (br d, J=6.2 Hz, 3H). MS (ESI) m/z 208.0 [M+H]⁺

Step 2: To a solution of (R)-2-(2-methylpyrrolidin-1-yl)-5-nitropyridine (900 mg, 4.34 mmol, 1.00 eq) in tetrahydrofuran (10.0 mL) was added Pd/C (100 mg, 10% purity) in portions under nitrogen. The mixture was stirred at 20° C. for 1 h under hydrogen (15 Psi). The mixture was filtered and the filtrate was concentrated to give (R)-6-(2-methylpyrrolidin-1-yl)pyridin-3-amine. MS (ESI) m/z 178.2 [M+H]⁺

Step 3: To a solution of (R)-6-(2-methylpyrrolidin-1-yl)pyridin-3-amine (770 mg, 4.34 mmol, 1.00 eq) and pyridine (1.72 g, 21.7 mmol, 1.75 mL, 5.00 eq) in Acetonitrile (10.0 mL) was added phenyl carbonochloridate (884 mg, 5.65 mmol, 707 uL, 1.30 eq) dropwise at 0° C. The mixture was stirred at 20° C. for 2 h. The mixture was concentrated and purified by reversed-phase HPLC to give (R)-phenyl (6-(2-methylpyrrolidin-1-yl)pyridin-3-yl)carbamate. MS (ESI) m/z 298.1 [M+H]⁺

Step 4: To a solution of 3-(6-(hydroxymethyl)-1-oxoisoindolin-2-yl)piperidine-2,6-dione VIII (80.0 mg, 292 umol, 1.00 eq) and (R)-phenyl (6-(2-methylpyrrolidin-1-yl)pyridin-3-yl)carbamate (113 mg, 379 umol, 1.30 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched by 1 M hydrochloric acid, purified by prep-HPLC and lyophilized to give (2-(2,6-dioxopiperidin-3-yl)-3-oxoisoindolin-5-yl)methyl (6-((R)-2-methylpyrrolidin-1-yl) pyridin-3-yl)carbamate 157. ¹H NMR (400 MHz, DMSO-d₆) δ=11.00 (s, 1H), 9.41 (br s, 1H), 8.16 (s, 1H), 8.09 (br s, 1H), 7.78 (s, 1H), 7.65 (q, J=7.8 Hz, 2H), 7.57 (br d, J=7.2 Hz, 1H), 6.41 (d, J=9.0 Hz, 1H), 5.24 (s, 2H), 5.13 (dd, J=5.0, 13.3 Hz, 1H), 4.53-4.42 (m, 1H), 4.40-4.30 (m, 1H), 4.10-4.01 (m, 1H), 3.43 (br d, J=2.3 Hz, 1H), 3.20 (br s, 1H), 2.96-2.87 (m, 1H), 2.63 (br d, J=2.6 Hz, 1H), 2.41 (br dd, J=4.3, 13.3 Hz, 1H), 2.07-1.96 (m, 3H), 1.95-1.87 (m, 1H), 1.69-1.59 (m, 1H), 1.13 (d, J=6.2 Hz, 3H). MS (ESI) m/z 478.2 [M+H]⁺

Compound 358: To a mixture of (1s,3s)-3-phenylcyclobutanamine (100 mg, 679 umol, 294 uL, 1.00 eq) and pyridine (161 mg, 2.04 mmol, 164 uL, 3.00 eq) in acetonitrile (2.00 mL) was added phenyl carbonochloridate (117 mg, 747 umol, 93.6 uL, 1.10 eq) dropwise. The mixture was stirred at 25° C. for 2 h. The mixture was concentrated to give crude product. The crude product was purified by reverse phase HPLC (column: spherical C18, 20-45 um, 100 Å, SW 120, mobile phase: [water (0.1% Formic Acid)-ACN]). The desired fraction was collected and lyophilized to give phenyl ((1 s,3s)-3-phenylcyclobutyl)carbamate (120 mg, 449 umol, 66% yield) as a yellow solid. MS (ESI) m/z 268.0 [M+H]⁺

To a mixture of phenyl ((1 s,3s)-3-phenylcyclobutyl)carbamate (120 mg, 449 umol, 1.00 eq) and 3-(6-(hydroxymethyl)-1-oxoisoindolin-2-yl)piperidine-2,6-dione 1 (148 mg, 539 umol, 1.20 eq) in dimethyl formamide (1.00 mL) was added sodium hydride (35.9 mg, 898 umol, 60% purity, 2.00 eq) at 0° C. The mixture was stirred at 20° C. for 1 h. The mixture was quenched with 1 M hydrochloric and filtered. The filtrate was purified by prep-HPLC (column: Phenomenex Synergi C18 150*25 mm*10 um; mobile phase: [water (0.225% FA)-ACN]; B %: 31%-61%, 10 min) and lyophilized to give (2-(2,6-dioxopiperidin-3-yl)-3-oxoisoindolin-5-yl)methyl ((1 s,3s)-3-phenylcyclobutyl)carbamate 358 (79.18 mg, 177 umol, 39% yield) as a white solid. ¹H NMR (400 MHz, DMSO-d₆) δ=10.98 (br s, 1H), 7.72 (s, 1H), 7.68 (br d, J=8.3 Hz, 1H), 7.62 (s, 2H), 7.32-7.24 (m, 4H), 7.22-7.16 (m, 1H), 5.18-5.12 (m, 3H), 4.50-4.44 (m, 1H), 4.38-4.30 (m, 1H), 4.06-3.96 (m, 1H), 3.14-3.04 (m, 1H), 2.98-2.88 (m, 1H), 2.66-2.58 (m, 3H), 2.46-2.38 (m, 1H), 2.08-1.98 (m, 3H). MS (ESI) m/z 448.1 [M+H]⁺

Compound 359: To a solution of 3-phenylcyclobutanamine (100 mg, 544 umol, 1.00 eq, hydrochloric acid) and pyridine (215 mg, 2.72 mmol, 219 uL, 5.00 eq) in acetonitrile (2.00 mL) was added phenyl carbonochloridate (102 mg, 653 umol, 81.8 uL, 1.20 eq), the mixture was stirred at 25° C. for 1 h. The reaction mixture was concentrated to give a residue. The residue was purified by reverse phase HPLC (column: spherical C18, 20-45 um, 100 Å, SW 120, mobile phase: [water (0.1% Formic Acid)-ACN) to afford phenyl ((1 r,3r)-3-phenylcyclobutyl)carbamate (100 mg, 374 umol, 68% yield) as a yellow solid. MS (ESI) m/z 268.1 [M+H]⁺

To a solution of phenyl ((1 r,3r)-3-phenylcyclobutyl)carbamate (93.6 mg, 350 umol, 1.20 eq) and 3-(6-(hydroxymethyl)-1-oxoisoindolin-2-yl)piperidine-2,6-dione 1 (80.0 mg, 291 umol, 1.00 eq) in dimethylformamide (1.00 mL) was added sodium hydride (23.3 mg, 583 umol, 60% purity, 2.00 eq). The mixture was stirred at 25° C. for 1 h. The reaction mixture was quenched with water (0.200 mL) and filtered to give a filtrate. The filtrate was purified by Prep-HPLC (column: Unisil 3-100 C18 Ultra 150*50 mm*3 um; mobile phase: [water (0.225% FA)-ACN]; B %: 35%-65%, 10 min) to afford (2-(2,6-dioxopiperidin-3-yl)-3-oxoisoindolin-5-yl)methyl((1 r,3r)-3-phenylcyclobutyl)carbamate 359 (70.16 mg, 156 umol, 53% yield) as a white solid. ¹H NMR (400 MHz, DMSO-d₆) δ=10.99 (br s, 1H), 7.84 (br d, J=7.2 Hz, 1H), 7.73 (s, 1H), 7.62 (s, 2H), 7.36-7.25 (m, 4H), 7.22-7.14 (m, 1H), 5.11 (br d, J=5.0 Hz, 3H), 4.52-4.41 (m, 1H), 4.39-4.28 (m, 1H), 4.20-4.06 (m, 1H), 3.58-3.46 (m, 1H), 2.99-2.86 (m, 1H), 2.62-2.57 (m, 1H), 2.41-2.30 (m, 5H), 2.06-1.96 (m, 1H). MS (ESI) m/z 448.0[M+H]⁺

Compound 360: To a solution of m-toluidine (200 mg, 1.87 mmol, 202 uL, 1.00 eq) in acetonitrile (5.00 mL) was added pyridine (443 mg, 5.60 mmol, 452 uL, 3.00 eq) and phenyl carbonochloridate (351 mg, 2.24 mmol, 280 uL, 1.20 eq). The mixture was stirred at 25° C. for 1 h. The mixture was filtered to give filtrate. The filtrate was purified by reverse phase HPLC (column: spherical C18, 20-45 um, 100 Å, SW 120, mobile phase: [water (0.1% Formic Acid)-ACN). The desired fraction was collected and concentrated to give phenyl m-tolylcarbamate (395 mg, 1.56 mmol, 84% yield, 90% purity) as a white solid. MS (ESI) m/z 228.0 [M+H]⁺

To a solution of 3-(6-(hydroxymethyl)-1-oxoisoindolin-2-yl)piperidine-2,6-dione I (80.0 mg, 292 umol, 1.00 eq) in dimethyl formamide (2.00 mL) was added phenyl m-tolylcarbamate (79.5 mg, 350 umol, 1.20 eq) and sodium hydride (23.3 mg, 583 umol, 60% purity, 2.00 eq) at 0° C. The mixture was stirred at 0° C. for 1 h. The mixture was adjusted pH=6 with formic acid (0.500 ml). The mixture was filtered to give filtrate. The filtrate was purified by prep-HPLC (column: Unisil 3-100 C18 Ultra 150*50 mm*3 um; mobile phase: [water (0.225% FA)-ACN]; B %: 30%-60%, 10 min). The desired fraction was collected and lyophilized to give (2-(2,6-dioxopiperidin-3-yl)-3-oxoisoindolin-5-yl)methyl m-tolylcarbamate 360 (62.88 mg, 152 umol, 52% yield, 99% purity) as a white solid. ¹H NMR (400 MHz, DMSO-d₆) δ=11.14-10.74 (m, 1H), 9.73 (s, 1H), 7.79 (s, 1H), 7.71-7.66 (m, 1H), 7.65-7.59 (m, 1H), 7.30 (s, 1H), 7.26 (br d, J=8.3 Hz, 1H), 7.15 (t, J=7.8 Hz, 1H), 6.81 (d, J=7.5 Hz, 1H), 5.26 (s, 2H), 5.13 (dd, J=5.1, 13.3 Hz, 1H), 4.51-4.42 (m, 1H), 4.38-4.29 (m, 1H), 2.97-2.86 (m, 1H), 2.60 (td, J=2.0, 15.3 Hz, 1H), 2.40 (dt, J=8.8, 13.2 Hz, 1H), 2.25 (s, 3H), 2.06-1.95 (m, 1H). MS (ESI) m/z 408.1 [M+H]⁺

Compound 361: To a solution of 4,6-dimethylpyridin-2-amine (1.00 g, 8.19 mmol, 1.00 eq) and pyridine (1.94 g, 24.5 mmol, 1.98 mL, 3.00 eq) in acetonitrile (10.0 mL) was added phenyl carbonochloridate (1.41 g, 9.00 mmol, 1.13 mL, 1.10 eq) dropwise at 0° C. The mixture was stirred at 20° C. for 1 h. The mixture was concentrated to give crude product and purified by silica gel chromatography (petroleum ether/ethyl acetate=1/0 to 20/1) to give phenyl (4,6-dimethylpyridin-2-yl)carbamate (600 mg, 2.48 mmol, 30% yield) as a white solid. MS (ESI) m/z 243.1 [M+H]⁺

To a solution of 3-(6-(hydroxymethyl)-1-oxoisoindolin-2-yl)piperidine-2,6-dione 1 (80.0 mg, 292 umol, 1.00 eq) and phenyl (4,6-dimethylpyridin-2-yl)carbamate (77.7 mg, 321 umol, 1.10 eq) in dimethyl formamide (1.00 mL) was added sodium hydride (23.3 mg, 583 umol, 60% purity, 2.00 eq) in portions at 0° C. The mixture was stirred at 0° C. for 1 h. The mixture was quenched by 1 M hydrochloric acid (1.00 mL) and filtered. The filtrate was purified by prep-HPLC (column: 3_Phenomenex Luna C18 75*30 mm*3 um; mobile phase: [water (0.05% HCl)-ACN]; B %: 18%-38%, 6.5 min) and lyophilized to give (2-(2,6-dioxopiperidin-3-yl)-3-oxoisoindolin-5-yl)methyl (4,6-dimethylpyridin-2-yl)carbamate 361 (34.72 mg, 79.7 umol, 27% yield, 97% purity) as a white solid. ¹H NMR (400 MHz, DMSO-d₆) δ=10.98 (s, 1H), 10.60 (br s, 1H), 7.80 (s, 1H), 7.71-7.62 (m, 2H), 7.50 (s, 1H), 6.94 (s, 1H), 5.32 (s, 2H), 5.12 (dd, J=5.1, 13.2 Hz, 1H), 4.50-4.45 (m, 1H), 4.36-4.32 (m, 1H), 2.96-2.85 (m, 1H), 2.63-2.57 (m, 1H), 2.42 (s, 3H), 2.39 (br d, J=4.5 Hz, 1H), 2.32 (s, 3H), 2.05-1.97 (m, 1H). MS (ESI) m/z 423.1 [M+H]⁺

Compound 362: To a solution of p-toluidine (200 mg, 1.87 mmol, 205 uL, 1.00 eq) in acetonitrile (5.00 mL) was added pyridine (443 mg, 5.60 mmol, 452 uL, 3.00 eq) and phenyl carbonochloridate (351 mg, 2.24 mmol, 281 uL, 1.20 eq). The mixture was stirred at 25° C. for 1 h. The mixture was filtered to give filtrate. The filtrate was purified by reversed-phase HPLC (column: spherical C18, 20-45 um, 100 Å, SW 120, mobile phase: [water (0.1% Formic acid)-ACN]). The desired fraction was collected and concentrated to give phenyl p-tolylcarbamate (375 mg, 1.49 mmol, 79% yield, 90% purity) as a white solid. MS (ESI) m/z 228.2 [M+H]⁺

To a solution of 3-(6-(hydroxymethyl)-1-oxoisoindolin-2-yl)piperidine-2,6-dione 1 (80.0 mg, 292 umol, 1.00 eq) in dimethyl formamide (2.00 mL) was added phenyl p-tolylcarbamate (79.5 mg, 350 umol, 1.20 eq) and sodium hydride (23.3 mg, 583 umol, 60% purity, 2.00 eq) at 0° C. The mixture was stirred at 0° C. for 1 h. The mixture was adjusted pH=6 with formic acid (0.500 mL). The mixture was filtered to give filtrate. The filtrate was purified by prep-HPLC (column: Phenomenex luna C18 150*25 mm*10 um; mobile phase: [water (0.225% FA)-ACN]; B %: 29%-59%, 10 min) and further purified by prep-HPLC (column: Phenomenex Synergi C18 150*25 mm*10 um; mobile phase: [water (0.225% FA)-ACN]; B %: 34%-54%, 10 min). The desired fraction was collected and lyophilized to give (2-(2,6-dioxopiperidin-3-yl)-3-oxoisoindolin-5-yl)methyl p-tolylcarbamate 362 (50.45 mg, 122 umol, 42% yield, 99% purity) as a white solid. ¹H NMR (400 MHz, DMSO-d₆) δ=10.95 (br s, 1H), 9.69 (br s, 1H), 7.79 (s, 1H), 7.71-7.60 (m, 2H), 7.35 (br d, J=8.1 Hz, 2H), 7.08 (br d, J=8.2 Hz, 2H), 5.25 (s, 2H), 5.13 (br dd, J=4.9, 13.3 Hz, 1H), 4.51-4.43 (m, 1H), 4.38-4.27 (m, 1H), 2.96-2.86 (m, 1H), 2.62 (br s, 1H), 2.40 (br dd, J=4.1, 13.1 Hz, 1H), 2.23 (s, 3H), 2.05-1.97 (m, 1H). MS (ESI) m/z 408.2 [M+H]⁺

Compound 363: To a solution of 5-chloro-2-fluoroaniline (0.500 g, 3.43 mmol, 1.00 eq), pyridine (0.80 g, 10.1 mmol, 816 uL, 2.94 eq) in acetonitrile (3.00 mL) was added phenyl carbonochloridate (600 mg, 3.83 mmol, 480 uL, 1.12 eq). The mixture was stirred at 25° C. for 16 h. The reaction mixture was concentrated under reduced pressure to give a residue. The residue was purified by reversed-phase HPLC (column: spherical C18, 20-45 um, 100 Å, SW 120, mobile phase: [water (0.1% Formic acid)-ACN). Compound phenyl (5-chloro-2-fluorophenyl)carbamate (171 mg, 643 umol, 19% yield) was obtained as a white solid. ¹H NMR (400 MHz, CDCl₃) δ=8.30-8.18 (m, 1H), 7.45-7.41 (m, 3H), 7.30 (br s, 1H), 7.21 (br d, J=7.7 Hz, 2H), 7.09-7.02 (m, 2H).

To a solution of phenyl (5-chloro-2-fluorophenyl)carbamate (85.3 mg, 321 umol, 1.10 eq) and 3-(6-(hydroxymethyl)-1-oxoisoindolin-2-yl)piperidine-2,6-dione 1 (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 29%-62%, 11 min) and lyophilized to afford (2-(2,6-dioxopiperidin-3-yl)-3-oxoisoindolin-5-yl)methyl (5-chloro-2-fluorophenyl)carbamate 363 (44.1 mg, 98.0 umol, 34% yield, 99% purity) as a white solid. ¹H NMR (400 MHz, DMSO-d₆) δ=11.00 (br s, 1H), 9.83 (br s, 1H), 7.89-7.78 (m, 2H), 7.70-7.62 (m, 2H), 7.29 (dd, J=8.9, 10.5 Hz, 1H), 7.20-7.15 (m, 1H), 5.29 (s, 2H), 5.13 (dd, J=5.0, 13.3 Hz, 1H), 4.50-4.44 (m, 1H), 4.37-4.31 (m, 1H), 2.92 (ddd, J=5.4, 13.5, 17.5 Hz, 1H), 2.60 (td, J=2.0, 15.4 Hz, 1H), 2.43-2.35 (m, 1H), 2.05-1.97 (m, 1H). MS (ESI) m/z 446.2 [M+H]⁺

Compound 364: To a solution of 3-fluoro-4-nitrophenol (500 mg, 3.18 mmol, 1.00 eq) in dichloromethane (10.0 mL) was added trifluoromethanesulfonic anhydride (1.35 g, 4.77 mmol, 788 uL, 1.50 eq) and triethylamine (966 mg, 9.55 mmol, 1.33 mL, 3.00 eq) at 0° C., the mixture was stirred at 0° C. for 1 h. The mixture was diluted with water (20.0 mL), extracted with ethyl acetate (3×30.0 mL). The combined organic layers were concentrated to give a residue. The residue was purified by silica gel chromatography (Petroleum ether/Ethyl acetate=0/1, 20/1) to afford 3-fluoro-4-nitrophenyl trifluoromethanesulfonate (900 mg, 3.11 mmol, 98% yield) as yellow oil. ¹H NMR (400 MHz, DMSO-d₆) δ=8.40 (t, J=8.9 Hz, 1H), 8.12 (dd, J=2.6, 11.1 Hz, 1H), 7.70-7.66 (m, 1H).

To a solution of 3-fluoro-4-nitrophenyl trifluoromethanesulfonate (800 mg, 2.77 mmol, 1.00 eq), cyclobutylboronic acid (415 mg, 4.15 mmol, 1.50 eq), cesium carbonate (2.00 M, 2.07 mL, 1.50 eq) in toluene (10.0 mL) were added [1,1-bis(diphenylphosphino)ferrocene]dichloropalladium(II) (162 mg, 221 umol, 0.080 eq). The mixture was stirred at 90° C. for 12 h under nitrogen atmosphere. The mixture was diluted with water (20.0 mL) and extracted with ethyl acetate (3×20.0 mL). The combined organic layers were dried over anhydrous sodium sulfate, filtered and concentrated under reduced pressure to give a residue. The residue was purified by silica gel chromatography (petroleum ether/ethyl acetate=1/0 to 1/1) to afford 4-cyclobutyl-2-fluoro-1-nitrobenzene (250 mg, 1.28 mmol, 46% yield) as yellow oil. ¹H NMR (400 MHz, DMSO-d₆) δ=8.10 (t, J=8.3 Hz, 1H), 7.46 (dd, J=1.5, 12.8 Hz, 1H), 7.32-7.28 (m, 1H), 3.66 (quin, J=8.8 Hz, 1H), 2.36-2.28 (m, 2H), 2.18-2.12 (m, 2H), 2.04-1.98 (m, 1H), 1.88-1.80 (m, 1H).

To a mixture of 4-cyclobutyl-2-fluoro-1-nitrobenzene (250 mg, 1.28 mmol, 1.00 eq) in methanol (20.0 mL) was added palladium on carbon (25.0 mg, 10% purity) in one portion under hydrogen atmosphere. The mixture was stirred at 20° C. for 1 h. The mixture was filtered to give filter liquor, the filter liquor was concentrated under reduced pressure to afford 4-cyclobutyl-2-fluoroaniline (170 mg, 1.03 mmol, 80% yield) as brown oil. MS (ESI) m/z. 166.2 [M+H]⁺

To a mixture of 4-cyclobutyl-2-fluoroaniline (170 mg, 1.03 mmol, 294 uL, 1.00 eq) and phenyl carbonochloridate (177 mg, 1.13 mmol, 142 uL, 1.10 eq) in acetonitrile (1.00 mL) was added pyridine (244 mg, 3.09 mmol, 249 uL, 3.00 eq). The mixture was stirred at 25° C. for 1 h. The reaction mixture was concentrated under reduced pressure to give a residue. The residue was purified by reversed phase-HPLC (column: spherical C18, 20-45 um, 100 Å, SW 120, mobile phase: [water (0.1% Formic Acid)-acetonitrile]) to afford phenyl (4-cyclobutyl-2-fluorophenyl)carbamate (210 mg, 736 umol, 72% yield) as a white solid. MS (ESI) m/z. 286.1 [M+H]⁺

To a mixture of phenyl (4-cyclobutyl-2-fluorophenyl)carbamate (125 mg, 438 umol, 1.20 eq) and 3-(6-(hydroxymethyl)-1-oxoisoindolin-2-yl)piperidine-2,6-dione 1 (100 mg, 365 umol, 1.00 eq) in dimethylformamide (1.00 mL) was added sodium hydride (29.2 mg, 729 umol, 60% purity, 2.00 eq) at 0° C. The mixture was stirred at 25° C. for 1 h. The reaction mixture was added hydrochloric acid (1 M, 2.00 mL) and filtered to give a filtrate. The filtrate was purified by Prep-HPLC (column: Phenomenex Luna C18 150*25 mm*10 um; mobile phase: [water (0.225% FA)-ACN]; B %: 38%-68%, 10 min) to afford (2-(2,6-dioxopiperidin-3-yl)-3-oxoisoindolin-5-yl)methyl (4-cyclobutyl-2-fluorophenyl)carbamate 364 (61.21 mg, 132 umol, 36% yield) as a white solid. ¹H NMR (400 MHz, DMSO-d₆) δ=10.98 (br s, 1H), 9.42 (br s, 1H), 7.80 (s, 1H), 7.68-7.62 (m, 2H), 7.52 (brt, J=8.0 Hz, 1H), 7.08 (dd, J=1.8, 12.0 Hz, 1H), 7.02 (dd, J=1.5, 8.3 Hz, 1H), 5.26 (s, 2H), 5.14 (dd, J=5.1, 13.4 Hz, 1H), 4.52-4.44 (m, 1H), 4.38-4.32 (m, 1H), 3.56-3.44 (m, 1H), 2.98-2.86 (m, 1H), 2.66-2.58 (m, 1H), 2.42 (dd, J=4.5, 13.1 Hz, 1H), 2.32-2.24 (m, 2H), 2.08-2.04 (m, 2H), 2.04-1.98 (m, 1H), 1.96-1.90 (m, 1H), 1.84-1.78 (m, 1H). MS (ESI) m/z. 466.1 [M+H]⁺

Compound 365: To a solution of 4-chloro-3-methylaniline (500 mg, 3.53 mmol, 1.00 eq), pyridine (279 mg, 3.53 mmol, 285 uL, 1.00 eq) in acetonitrile (10.0 mL) was added phenyl carbonochloridate (608 mg, 3.88 mmol, 486 uL, 1.10 eq) at 0° C. The mixture was stirred at 20° C. for 1 h. The reaction mixture was concentrated under reduced pressure to give a residue. The residue was purified by reversed-phase HPLC (column: spherical C18, 20-45 um, 100 Å, SW 80, mobile phase: [water (0.1% Formic acid)-ACN]). Compound phenyl (4-chloro-3-methylphenyl)carbamate (1.34 g, 5.07 mmol, 72% yield, 99% purity) was obtained as a white solid. MS (ESI) m/z 262.0 [M+H]⁺

To a solution of phenyl (4-chloro-3-methylphenyl)carbamate (84.0 mg, 321 umol, 1.10 eq) and 3-(6-(hydroxymethyl)-1-oxoisoindolin-2-yl)piperidine-2,6-dione 1 (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 32%-62%, 10 min) and lyophilized to afford (2-(2,6-dioxopiperidin-3-yl)-3-oxoisoindolin-5-yl)methyl (4-chloro-3-methylphenyl)carbamate 365 (83.9 mg, 189 umol, 64% yield, 99% purity) as a white solid. ¹H NMR (400 MHz, DMSO-d₆) δ=10.98 (br s, 1H), 9.88 (s, 1H), 7.79 (s, 1H), 7.70-7.61 (m, 2H), 7.45 (s, 1H), 7.37-7.26 (m, 2H), 5.27 (s, 2H), 5.12 (dd, J=5.1, 13.3 Hz, 1H), 4.51-4.42 (m, 1H), 4.37-4.30 (m, 1H), 2.97-2.85 (m, 1H), 2.60 (td, J=2.0, 15.3 Hz, 1H), 2.46-2.37 (m, 1H), 2.28 (s, 3H), 2.05-1.97 (m, 1H). MS (ESI) m/z 442.1 [M+H]⁺

Compound 366: To a solution of 1,3-difluoro-5-methyl-2-nitrobenzene (500 mg, 2.89 mmol, 1.00 eq) in tetrahydrofuran (5.00 mL) was added palladium/carbon (50.0 mg, 10% purity) under nitrogen. The mixture was stirred at 20° C. for 1 h under hydrogen (15 Psi). The mixture was filtered and the filtrate was concentrated under reduced pressure to give 2,6-difluoro-4-methylaniline (386 mg, 2.70 mmol, 93% yield) as yellow oil.

¹H NMR (400 MHz, DMSO-d₆) δ=6.71-6.57 (m, 2H), 4.80 (br s, 2H), 2.09 (s, 3H). MS (ESI) m/z 144.2 [M+H]⁺.

To a solution of 2,6-difluoro-4-methylaniline (350 mg, 2.45 mmol, 1.00 eq) and pyridine (580 mg, 7.34 mmol, 592 uL, 3.00 eq) in acetonitrile (5.00 mL) was added phenyl carbonochloridate (421 mg, 2.69 mmol, 337 uL, 1.10 eq) dropwise at 0° C. The mixture was then stirred at 25° C. for 1 h. The reaction mixture was concentrated under reduced pressure to give crude product. The crude product was purified by reversed-phase HPLC (column: spherical C18, 20-45 um, 100 Å, SW 40, mobile phase: [water (0.1% Formic Acid)-ACN]) and lyophilized to give phenyl (2,6-difluoro-4-methylphenyl) carbamate (435 mg, 1.65 mmol, 68% yield) as a white solid. ¹H NMR (400 MHz, DMSO-d₆) δ=9.32 (s, 1H), 7.32-7.09 (m, 5H), 6.77-6.74 (m, 2H), 2.33 (s, 3H). MS (ESI) m/z 264.0 [M+H]⁺.

To a solution of phenyl (2,6-difluoro-4-methylphenyl)carbamate (92.1 mg, 350 umol, 1.20 eq) and 3-(6-(hydroxymethyl)-1-oxoisoindolin-2-yl)piperidine-2,6-dione (80.0 mg, 292 umol, 1.00 eq) in dimethyl formamide (1.00 mL) was added sodium hydride (23.3 mg, 583 umol, 60% purity, 2.00 eq) in portions at 0° C. The mixture was stirred at 20° C. for 1 h. The mixture was quenched by 1 M hydrochloric acid (1.00 mL) and filtered. The filtrate was purified by prep-HPLC (column: 3_Phenomenex Luna C18 75*30 mm*3 um; mobile phase: [water (0.05% HCl)-ACN]; B %: 35%-45%, 6 min) to give (2-(2,6-dioxopiperidin-3-yl)-3-oxoisoindolin-5-yl)methyl (2,6-difluoro-4-methylphenyl)carbamate 366 (71.1 mg, 157 umol, 54% yield, 99% purity) as a white solid.

¹H NMR (400 MHz, DMSO-d₆) δ=11.01 (s, 1H), 9.22 (brd, J=1.5 Hz, 1H), 7.76 (br s, 1H), 7.64 (s, 2H), 7.00 (d, J=8.7 Hz, 2H), 5.25 (s, 2H), 5.13 (dd, J=5.0, 13.3 Hz, 1H), 4.52-4.43 (m, 1H), 4.39-4.31 (m, 1H), 2.98-2.86 (m, 1H), 2.61 (br d, J=17.6 Hz, 1H), 2.41 (br dd, J=4.5, 13.1 Hz, 1H), 2.32 (s, 3H), 2.06-1.97 (m, 1H). MS (ESI) m/z 444.1 [M+H]⁺.

Compound 367: To a solution of bromobenzene (1.00 g, 6.37 mmol, 671 uL, 1.00 eq), tert-butyl azetidin-3-ylcarbamate (2.66 g, 12.7 mmol, 2.00 eq, hydrochloric acid) and cesium carbonate (6.23 g, 19.1 mmol, 3.00 eq) in dioxane (20.0 mL) was added 4,5-bis(diphenylphosphino)-9,9-dimethylxanthene (553 mg, 955 umol, 0.15 eq) and tris(dibenzylideneacetone)dipalladium(0) (292 mg, 318 umol, 0.05 eq). The mixture was stirred at 90° C. for 12 h under nitrogen atmosphere. The reaction mixture was quenched by addition water (50.0 mL), and extracted with ethyl acetate (2×100 mL). The combined organic layers were dried over anhydrous sodium sulfate, filtered and concentrated under reduced pressure to give a residue. The residue was purified by column chromatography (SiO₂, petroleum ether/ethyl acetate=1/0 to 10/1) to give tert-butyl (1-phenylazetidin-3-yl)carbamate (1.20 g, 4.83 mmol, 76% yield) as a yellow solid. ¹H NMR (400 MHz, DMSO-d₆) δ=7.50 (br d, J=7.2 Hz, 1H), 7.15 (t, J=7.8 Hz, 2H), 6.67 (t, J=7.3 Hz, 1H), 6.41 (d, J=7.9 Hz, 2H), 4.47-4.33 (m, 1H), 4.04 (t, J=7.3 Hz, 2H), 3.53 (t, J=6.8 Hz, 2H), 1.39 (s, 9H).

To a solution of tert-butyl (1-phenylazetidin-3-yl)carbamate (1.20 g, 4.83 mmol, 1.00 eq) in dichloromethane (20.0 mL) was added trifluoroacetic acid (6.16 g, 54.0 mmol, 4.00 mL, 11.2 eq). The mixture was stirred at 20° C. for 1 h. The reaction mixture was concentrated under reduced pressure to give 1-phenylazetidin-3-amine (700 mg, crude) as yellow oil. ¹H NMR (400 MHz, DMSO-d₆) δ=8.33 (br s, 2H), 7.19 (dd, J=7.5, 8.4 Hz, 2H), 6.73 (t, J=7.3 Hz, 1H), 6.50 (d, J=7.6 Hz, 2H), 4.14-4.04 (m, 3H), 3.81-3.73 (m, 2H).

To a solution of 1-phenylazetidin-3-amine (700 mg, 4.72 mmol, 1.00 eq) in acetonitrile (20.0 mL) was added pyridine (2.94 g, 37.2 mmol, 3.00 mL, 7.87 eq). Then phenyl carbonochloridate (813 mg, 5.20 mmol, 650 uL, 1.10 eq) was added into the mixture at 0° C. The mixture was stirred at 20° C. for 1 h. The reaction mixture was concentrated under reduced pressure to give a residue. The residue was purified by reversed-phase HPLC (column: spherical C18, 20-45 um, 100 Å, SW 120, mobile phase: [water (0.1% Formic Acid)-ACN]) to give phenyl (1-phenylazetidin-3-yl)carbamate (1.01 g, 2.94 mmol, 62% yield, 78% purity) as a white solid. MS (ESI) m/z 269.0 [M+H]⁺

To a solution of phenyl (1-phenylazetidin-3-yl)carbamate (86.1 mg, 321 umol, 1.10 eq) and 3-(6-(hydroxymethyl)-1-oxoisoindolin-2-yl)piperidine-2,6-dione 1 (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 24%-54%, 10 min), and lyophilized to afford (2-(2,6-dioxopiperidin-3-yl)-3-oxoisoindolin-5-yl)methyl(1-phenylazetidin-3-yl)carbamate 367 (62.7 mg, 138 umol, 47% yield, 99% purity, formate) as a white solid. ¹H NMR (400 MHz, DMSO-d6) δ=10.99 (br s, 1H), 8.01 (br d, J=7.6 Hz, 1H), 7.72 (s, 1H), 7.61 (s, 2H), 7.15 (t, J=7.9 Hz, 2H), 6.68 (t, J=7.3 Hz, 1H), 6.43 (br d, J=7.8 Hz, 2H), 5.18-5.09 (m, 3H), 4.50-4.42 (m, 2H), 4.35-4.28 (m, 1H), 4.08 (t, J=7.3 Hz, 2H), 3.57 (brt, J=6.7 Hz, 2H), 2.96-2.86 (m, 1H), 2.63-2.57 (m, 1H), 2.40 (br d, J=8.6 Hz, 1H), 2.04-1.97 (m, 1H). MS (ESI) m/z 449.3 [M+H]⁺

Compound 368: To a solution of tert-butyl (1-benzylazetidin-3-yl)carbamate (0.500 g, 1.91 mmol, 1.00 eq) in dichloromethane (5.00 mL) was added trifluoroacetic acid (1.54 g, 13.5 mmol, 1.00 mL, 7.09 eq). The mixture was stirred at 25° C. for 12 h. The mixture was concentrated to give 1-benzylazetidin-3-amine (250 mg, 1.54 mmol, 80% yield) as yellow oil. ¹H NMR (400 MHz, DMSO-d₆) δ=8.55 (br s, 2H), 7.46 (s, 5H), 4.43 (s, 2H), 4.33-4.11 (m, 5H).

To a solution of 1-benzylazetidin-3-amine (250 mg, 1.54 mmol, 1.00 eq) in acetonitrile (10.0 mL) was added pyridine (609 mg, 7.71 mmol, 622 uL, 5.00 eq) and phenyl carbonochloridate (289 mg, 1.85 mmol, 232 uL, 1.20 eq). The mixture was stirred at 25° C. for 1 h. The mixture was filtered to give filtrate. The filtrate was purified by reversed-phase HPLC (column: spherical C18, 20-45 um, 100 Å, SW 120, mobile phase: [water (0.1% Formic Acid)-ACN]). The desired fraction was collected and concentrated to give phenyl (1-benzylazetidin-3-yl)carbamate (390 mg, 1.24 mmol, 80% yield, 90% purity) as yellow oil. ¹H NMR (400 MHz, DMSO-d₆) δ=8.15 (s, 1H), 7.40-7.17 (m, 10H), 4.18 (br d, J=7.1 Hz, 1H), 3.66 (s, 2H), 3.61 (brd, J=8.4 Hz, 2H), 3.10 (br d, J=6.4 Hz, 2H).

To a solution of 3-(6-(hydroxymethyl)-1-oxoisoindolin-2-yl)piperidine-2,6-dione 1 (100 mg, 365 umol, 1.00 eq) in dimethyl formamide (1.00 mL) was added phenyl (1-benzylazetidin-3-yl)carbamate (144 mg, 510 umol, 1.40 eq) and sodium hydride (29.2 mg, 729 umol, 60% purity, 2.00 eq) at 0° C. The mixture was stirred at 0° C. for 1 h. The mixture was adjusted pH=6 with formic acid (0.500 mL). The mixture was filtered to give filtrate. The filtrate was purified by prep-HPLC (column: Phenomenex Synergi C18 150*25 mm*10 um; mobile phase: [water (0.225% FA)-ACN]; B %: 2%-32%, 10 min) and further purified by prep-NPLC (column: Welch Ultimate XB-SiOH 250*50*10 um; mobile phase: [Hexane-EtOH]; B %: 15%-55%, 15 min). The desired fraction was collected and concentrated to give a residue. The residue was purified by prep-HPLC (column: Unisil 3-100 C18 Ultra 150*50 mm*3 um; mobile phase: [water (0.225% FA)-ACN]; B %: 2%-32%, 10 min). The desired fraction was collected and lyophilized to give (2-(2,6-dioxopiperidin-3-yl)-3-oxoisoindolin-5-yl)methyl(1-benzylazetidin-3-yl)carbamate 368 (53.91 mg, 115 umol, 32% yield, 99% purity) as an off-white solid. ¹H NMR (400 MHz, DMSO-d₆) δ=10.99 (s, 1H), 8.17 (s, 1H), 7.84 (br d, J=7.3 Hz, 1H), 7.70 (s, 1H), 7.60 (s, 2H), 7.33-7.26 (m, 2H), 7.26-7.20 (m, 2H), 5.24-4.88 (m, 3H), 4.49-4.40 (m, 1H), 4.37-4.27 (m, 1H), 4.19-4.05 (m, 1H), 3.62-3.41 (m, 4H), 2.99-2.78 (m, 3H), 2.60 (br dd, J=1.8, 15.7 Hz, 1H), 2.40 (br dd, J=4.5, 13.1 Hz, 1H), 2.07-1.94 (m, 1H). MS (ESI) m/z 463.1 [M+H]⁺

Compound 369: To a mixture of 2-fluoro-3-(trifluoromethoxy)aniline (500 mg, 2.56 mmol, 1.00 eq) and pyridine (608 mg, 7.69 mmol, 620 uL, 3.00 eq) in acetonitrile (5.00 mL) was added phenyl carbonochloridate (521 mg, 3.33 mmol, 417 uL, 1.30 eq) dropwise. The mixture was stirred at 15° C. for 12 h. The mixture was concentrated to give crude product. The crude product was purified by reversed-phase HPLC (column: spherical C18, 20-45 um, 100 Å, SW 80, mobile phase: [water (0.1% Formic Acid)-ACN]). The desired fraction was collected and lyophilized to give phenyl (2-fluoro-3-(trifluoromethoxy)phenyl)carbamate (600 mg, 1.90 mmol, 74% yield) as a white solid. ¹H NMR (400 MHz, DMSO-d₆) δ=10.29 (br s, 1H), 7.81-7.75 (m, 1H), 7.48 (s, 1H), 7.35-7.31 (m, 2H), 7.30 (br d, J=1.1 Hz, 1H), 7.25 (br t, J=7.6 Hz, 3H).

To a mixture of 3-(6-(hydroxymethyl)-1-oxoisoindolin-2-yl)piperidine-2,6-dione 1 (80.0 mg, 291 umol, 1.00 eq) and phenyl (2-fluoro-3-(trifluoromethoxy)phenyl)carbamate (110 mg, 350 umol, 1.20 eq) in dimethyl formamide (1.00 mL) was added sodium hydride (17.5 mg, 437 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by Prep-HPLC (column: 3_Phenomenex Luna C18 75*30 mm*3 um; mobile phase: [water (0.05% HCl)-ACN]; B %: 44%-54%, 6 min) and lyophilization to give (2-(2,6-dioxopiperidin-3-yl)-3-oxoisoindolin-5-yl)methyl(2-fluoro-3-(trifluoromethoxy)phenyl)carbamate 369 (13.99 mg, 27.9 umol, 9% yield, 99% purity) as a white solid. ¹H NMR (400 MHz, DMSO-d₆) δ=10.99 (br s, 1H), 9.87 (br s, 1H), 7.81 (s, 1H), 7.74 (brt, J=7.5 Hz, 1H), 7.70-7.66 (m, 1H), 7.66-7.61 (m, 1H), 7.31-7.24 (m, 2H), 5.30 (s, 2H), 5.13 (dd, J=5.0, 13.3 Hz, 1H), 4.50-4.44 (m, 1H), 4.37-4.31 (m, 1H), 2.94-2.86 (m, 1H), 2.62-2.59 (m, 1H), 2.40 (br dd, J=4.2, 13.1 Hz, 1H), 2.05-1.97 (m, 1H). MS (ESI) m/z 496.0 [M+H]⁺

Compound 370: To a mixture of 6-(tert-butyl)pyridin-2-amine (100 mg, 665 umol, 1.00 eq) and pyridine (157 mg, 2.00 mmol, 161 uL, 3.00 eq) in acetonitrile (2.00 mL) was added phenyl carbonochloridate (135 mg, 865 umol, 108 uL, 1.30 eq) dropwise. The mixture was stirred at 15° C. for 2 h. The mixture was concentrated to give crude product. The crude product was purified by reversed-phase HPLC (column: spherical C18, 20-45 um, 100 Å, SW 20, mobile phase: [water (0.1% Formic Acid)-ACN]). The desired fraction was collected and lyophilized to give phenyl (6-(tert-butyl)pyridin-2-yl)carbamate (85.0 mg, 314 umol, 47% yield) as a white solid. ¹H NMR (400 MHz, DMSO-d₆) δ=10.43 (s, 1H), 7.70 (d, J=7.9 Hz, 2H), 7.61-7.57 (m, 1H), 7.46-7.40 (m, 2H), 7.23-7.19 (m, 2H), 7.18-7.16 (m, 1H), 1.30 (s, 9H).

To a mixture of 3-(6-(hydroxymethyl)-1-oxoisoindolin-2-yl)piperidine-2,6-dione 1 (70.0 mg, 255 umol, 1.00 eq) and phenyl (6-(tert-butyl)pyridin-2-yl)carbamate (82.7 mg, 306 umol, 1.20 eq) in dimethyl formamide (0.500 mL) was added sodium hydride (15.3 mg, 382 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC (column: 3_Phenomenex Luna C18 75*30 mm*3 um; mobile phase: [water (0.05% HCl)-ACN]; B %: 24%-34%, 6 min) followed by prep-HPLC (column: Phenomenex Synergi C18 150*25 mm*10 um; mobile phase: [water (0.225% FA)-ACN]; B %: 32%-52%, 10 min) and lyophilized to give (2-(2,6-dioxopiperidin-3-yl)-3-oxoisoindolin-5-yl)methyl (6-(tert-butyl)pyridin-2-yl)carbamate 370 (29.83 mg, 64.2 umol, 25% yield, 97% purity) as a white solid. ¹H NMR (400 MHz, DMSO-d₆) δ=10.99 (br s, 1H), 10.08 (s, 1H), 7.84 (s, 1H), 7.68 (t, J=7.7 Hz, 2H), 7.65-7.59 (m, 2H), 7.07 (d, J=7.5 Hz, 1H), 5.29 (s, 2H), 5.13 (dd, J=5.1, 13.3 Hz, 1H), 4.49-4.44 (m, 1H), 4.38-4.31 (m, 1H), 2.91 (ddd, J=5.3, 13.4, 17.5 Hz, 1H), 2.61 (br s, 1H), 2.45-2.36 (m, 1H), 2.05-1.98 (m, 1H), 1.28 (s, 9H). MS (ESI) m/z 451.2 [M+H]⁺

Compound 371: To a solution of 1-phenylcyclopropanamine (200 mg, 1.18 mmol, 1.00 eq, hydrochloric acid) and pyridine (280 mg, 3.54 mmol, 285 uL, 3.00 eq) in acetonitrile (5.00 mL) was added phenyl carbonochloridate (203 mg, 1.30 mmol, 162 uL, 1.10 eq) at 0° C. The mixture was stirred at 20° C. for 1 h. The reaction mixture was concentrated under reduced pressure to give a residue. The residue was purified by reversed-phase HPLC (C18, 80 g; condition: water/acetonitrile=1/0 to 1/4, 0.1% formic acid) to give phenyl (1-phenylcyclopropyl)carbamate (220 mg, 799 umol, 68% yield, 92% purity) as a white solid. ¹H NMR (400 MHz, DMSO-d₆) δ=8.62 (s, 1H), 7.42-7.26 (m, 5H), 7.25-7.16 (m, 4H), 7.11 (d, J=7.6 Hz, 1H), 1.28-1.23 (m, 2H), 1.23-1.16 (m, 2H). MS (ESI) m/z 254.0 [M+H]⁺

A mixture of phenyl (1-phenylcyclopropyl)carbamate (81.3 mg, 321 umol, 1.10 eq), 3-(6-(hydroxymethyl)-1-oxoisoindolin-2-yl)piperidine-2,6-dione 1 (80.0 mg, 292 umol, 1.00 eq) in dimethyl formamide (1.50 mL) was added sodium hydride (23.3 mg, 583 umol, 60% purity, 2.00 eq) at 0° C. And then the mixture was stirred at 20° C. for 1 h. The pH of the mixture was adjusted to around 6 by adding hydrochloric acid. The mixture was extracted with ethyl acetate (3×10 mL). The combined organic layers were dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 28%-50%, 11 min) and lyophilized to give a white solid. The white solid was re-purified by column chromatography (SiO₂, Petroleum ether/Ethyl acetate=1/0 to 5/1) to afford (2-(2,6-dioxopiperidin-3-yl)-3-oxoisoindolin-5-yl)methyl (1-phenylcyclopropyl)carbamate 371 (13.2 mg, 28.8 umol, 10% yield, 95% purity) as a white solid. ¹H NMR (400 MHz, DMSO-d₆) δ=11.16-10.78 (m, 1H), 8.21 (s, 1H), 7.74 (s, 1H), 7.61 (s, 2H), 7.30-7.23 (m, 2H), 7.22-7.07 (m, 3H), 5.18-5.07 (m, 3H), 4.50-4.42 (m, 1H), 4.38-4.27 (m, 1H), 2.96-2.87 (m, 1H), 2.62 (br d, J=2.4 Hz, 1H), 2.44-2.36 (m, 1H), 2.06-1.97 (m, 1H), 1.16 (br d, J=5.0 Hz, 4H). MS (ESI) m/z 434.3 [M+H]⁺

Compound 372: To a solution of 1-(tert-butyl)azetidin-3-amine (500 mg, 3.90 mmol, 1.00 eq) and pyridine (925 mg, 11.7 mmol, 944 uL, 3.00 eq) in acetonitrile (10.0 mL) was added phenyl carbonochloridate (672 mg, 4.29 mmol, 537 uL, 1.10 eq) at 0° C. The mixture was stirred at 20° C. for 1 h. The reaction mixture was concentrated under reduced pressure to give a residue. The residue was purified by reversed-phase HPLC (C18, 80 g; condition: water/acetonitrile=1/0 to 8/1, 0.1% formic acid) to give phenyl (1-(tert-butyl)azetidin-3-yl) carbamate (595 mg, 2.01 mmol, 52% yield, 84% purity) as a white solid. ¹H NMR (400 MHz, DMSO-d₆) δ=8.11-7.89 (m, 1H), 7.42-7.37 (m, 2H), 7.25-7.20 (m, 1H), 7.12 (br d, J=7.7 Hz, 2H), 4.40-4.35 (m, 1H), 4.10-3.95 (m, 4H), 1.27-1.22 (m, 9H). MS (ESI) m/z 249.2 [M+H]⁺

To a solution of phenyl (1-(tert-butyl)azetidin-3-yl)carbamate (145 mg, 583 umol, 1.00 eq) and 3-(6-(hydroxymethyl)-1-oxoisoindolin-2-yl)piperidine-2,6-dione 1 (160 mg, 583 umol, 1.00 eq) in dimethyl formamide (3.00 mL) was added sodium hydride (46.7 mg, 1.17 mmol, 60% purity, 2.00 eq) at 0° C. The mixture was stirred at 0° C. for 16 h. The pH of the mixture was adjusted to around 6 by adding hydrochloric acid. The mixture was filtered and concentrated under reduced pressure to give a residue. The residue was purified by prep-HPLC (column: Phenomenex Synergi C18 150*25 mm*10 um; mobile phase: [water (0.225% FA)-ACN]; B %: 0%-25%, 10 min) and lyophilized to afford (2-(2,6-dioxopiperidin-3-yl)-3-oxoisoindolin-5-yl)methyl (1-(tert-butyl)azetidin-3-yl)carbamate 372 (97.2 mg, 193 umol, 33% yield, 94% purity, formate) as a white solid. ¹H NMR (400 MHz, DMSO-d₆) δ=10.99 (s, 1H), 7.82 (brd, J=6.0 Hz, 1H), 7.71 (s, 1H), 7.60 (s, 2H), 5.15-5.08 (m, 3H), 4.48-4.43 (m, 1H), 4.35-4.30 (m, 1H), 4.05-4.01 (m, 1H), 3.38 (br d, J=4.5 Hz, 2H), 3.10 (br d, J=3.5 Hz, 2H), 2.95-2.87 (m, 1H), 2.60 (br d, J=17.5 Hz, 1H), 2.40 (br d, J=8.8 Hz, 1H), 2.04-1.97 (m, 1H), 0.92 (br s, 9H). MS (ESI) m/z 429.0 [M+H]⁺

Compound 373: To a solution of 2,3-dihydrobenzofuran-7-amine (500 mg, 3.70 mmol, 1.00 eq) and pyridine (877 mg, 11.1 mmol, 895 uL, 3.00 eq) in acetonitrile (5.00 mL) was added phenyl carbonochloridate (695 mg, 4.44 mmol, 556 uL, 1.20 eq). The reaction mixture was stirred at 20° C. for 1 h. The reaction mixture was concentrated to give a residue, which was poured into water (100 mL) and extracted with ethyl acetate (3×50.0 mL). The combined organic phase was separated, washed with brine (20.0 mL), dried over anhydrous sodium sulfate, filtered and concentrated to give phenyl (2,3-dihydrobenzofuran-7-yl)carbamate (950 mg, crude) as colorless oil.

A mixture of 3-(6-(hydroxymethyl)-1-oxoisoindolin-2-yl)piperidine-2,6-dione 1 (80.0 mg, 291 umol, 1.00 eq) in dimethyl formamide (1.00 mL) was added sodium hydride (23.3 mg, 583 umol, 60% purity, 2.00 eq) and phenyl (2,3-dihydrobenzofuran-7-yl)carbamate (89.3 mg, 350 umol, 1.20 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 um; mobile phase: [water (0.225% formic acid)-acetonitrile]; B %: 27%-57%, 10 min) and lyophilized to afford (2-(2,6-dioxopiperidin-3-yl)-3-oxoisoindolin-5-yl)methyl(2,3-dihydrobenzofuran-7-yl)carbamate 373 (90.38 mg, 203 umol, 69% yield, 98% purity) as a white solid. ¹H NMR (400 MHz, DMSO-d₆) δ=11.01 (s, 1H), 9.08 (br s, 1H), 7.80 (s, 1H), 7.64 (q, J=8.0 Hz, 2H), 7.28 (br s, 1H), 7.00 (d, J=7.2 Hz, 1H), 6.83-6.74 (m, 1H), 5.24 (s, 2H), 5.14 (dd, J=5.2, 13.2 Hz, 1H), 4.55 (t, J=8.8 Hz, 2H), 4.50-4.43 (m, 1H), 4.39-4.29 (m, 1H), 3.21 (t, J=8.8 Hz, 2H), 2.98-2.87 (m, 1H), 2.64-2.58 (m, 1H), 2.44-2.36 (m, 1H), 2.07-1.96 (m, 1H). MS (ESI) m/z 436.1 [M+H]⁺.

Compound 374: To a solution of 1-bromo-2-fluorobenzene (32.2 g, 184 mmol, 20.1 mL, 2.10 eq) in tetrahydrofuran (200 mL) was added n-butyllithium (2.50 M, 73.6 mL, 2.10 eq) at −78° C. under nitrogen atmosphere, the reaction was stirred at −78° C. for 1.5 h. A solution of 3-oxocyclobutanecarboxylic acid (10.0 g, 87.6 mmol, 1.00 eq) in tetrahydrofuran (20.0 mL) was added to the reaction slowly. The mixture was stirred at same temperature for another 1 h. The reaction was quenched by saturated ammonium chloride (200 mL) and the mixture was acidified with 11.8 M concentrated hydrochloric acid to pH=3. The mixture was extracted with ethyl acetate (3×200 mL) and the combined organic layers were concentrated to afford 3-(2-fluorophenyl)-3-hydroxycyclobutanecarboxylic acid (16.5 g, 78.5 mmol, 89% yield) as a white solid. ¹H NMR (400 MHz, DMSO-d₆) δ=12.16 (br s, 1H), 7.59-7.44 (m, 1H), 7.40-7.29 (m, 1H), 7.22-7.11 (m, 2H), 5.67 (br s, 1H), 2.85-2.76 (m, 2H), 2.63-2.54 (m, 1H), 2.49-2.44 (m, 2H).

To a solution of 3-(2-fluorophenyl)-3-hydroxycyclobutanecarboxylic acid (16.5 g, 78.5 mmol, 1.00 eq) in toluene (120 mL) was added concentrated hydrochloric acid (12.0 M, 82.5 mL, 12.4 eq), the reaction was stirred at 20° C. for 4 h. The organic phase was separated, washed with water (20.0 mL), saturated sodium chloride solution (20.0 mL) and concentrated to give a residue. The residue was washed with petroleum ether (300 mL) to afford 3-chloro-3-(2-fluorophenyl)cyclobutanecarboxylic acid (16.3 g, 71.2 mmol, 90% yield) as a brown solid. ¹H NMR (400 MHz, DMSO-d₆) δ=12.43 (br s, 1H), 7.47-7.35 (m, 2H), 7.27-7.20 (m, 2H), 3.56 (quin, J=8.8 Hz, 1H), 2.98 (br d, J=8.0 Hz, 4H).

To a solution of 3-chloro-3-(2-fluorophenyl)cyclobutanecarboxylic acid (16.3 g, 71.2 mmol, 1.00 eq) and potassium carbonate (21.6 g, 156 mmol, 2.20 eq) in dimethylformamide (120 mL) was added iodomethane (20.2 g, 142 mmol, 8.88 mL, 2.00 eq). The mixture was stirred at 25° C. for 12 h. The mixture was filtered and diluted with water (100 ml), extracted with ethyl acetate (3×50.0 mL). The combined organic layers were concentrated to give a residue. The residue was purified by column chromatography (ISCO®; 120 g SepaFlash® Silica Flash Column, Eluent of 0˜100% ethyl acetate/petroleum ether @ 80 mL/min) to afford methyl 3-chloro-3-(2-fluorophenyl)cyclobutanecarboxylate (10.7 g, 44.0 mmol, 61% yield) as yellow oil. ¹H NMR (400 MHz, DMSO-d₆) δ=7.45-7.37 (m, 2H), 7.29-7.20 (m, 2H), 3.70-3.63 (m, 1H), 3.59 (s, 3H), 3.08-2.95 (m, 4H).

To a solution of sodium bis(trimethylsilyl)amide (2.00 M, 15.4 mL, 1.50 eq) in tetrahydrofuran (25.0 mL) was added a solution of methyl 3-chloro-3-(2-fluorophenyl)cyclobutanecarboxylate (5.00 g, 20.6 mmol, 1.00 eq) in tetrahydrofuran (25.0 mL) dropwise. The mixture was stirred at 70° C. for 5 h. The reaction was quenched by ammonium chloride solution (100 mL) and the mixture was extracted with ethyl acetate (3×50 mL), the combined organic layers were dried over sodium sulfate and concentrated to give a residue. The residue was purified by column chromatography (ISCO®; 120 g SepaFlash® Silica Flash Column, Eluent of 0˜100% ethyl acetate/petroleum ether @ 40 mL/min) to afford methyl 3-(2-fluorophenyl)bicyclo[1.1.0] butane-1-carboxylate (2.70 g, 13.0 mmol, 63% yield) as yellow oil. ¹H NMR (400 MHz, DMSO-d₆) δ=7.40-7.35 (m, 1H), 7.34-7.28 (m, 1H), 7.23-7.16 (m, 2H), 3.50 (s, 3H), 2.87 (s, 2H), 1.67 (s, 2H).

A mixture of methyl 3-(2-fluorophenyl)bicyclo[1.1.0]butane-1-carboxylate (1.00 g, 4.85 mmol, 1.00 eq) and 1-methoxy-2-(2-methoxyethoxy)ethane (3.75 g, 27.9 mmol, 4.00 mL, 5.76 eq) in tetrachloroethylene (40.0 mL) was heated to 120° C., then sodium trichloroacetate (3.37 g, 18.1 mmol, 3.75 eq) was added in one portion. The mixture was stirred at 140° C. for 6 h. The reaction was filtered to give a filtrate, the filtrate was concentrated to give a residue. The residue was purified by Prep-HPLC (column: Phenomenex luna C18 150*25 mm*10 um; mobile phase: [water (0.225% FA)-ACN]; B %: 62%-92%, 10 min) to give a crude product. The crude product was purified by Prep-HPLC (column: Phenomenex luna C18 150*25 mm*10 um; mobile phase: [water (0.225% FA)-ACN]; B %: 49%-79%, 10 min) followed purified by Prep-HPLC (column: Phenomenex luna C18 150*25 mm*10 um; mobile phase: [water (0.225% FA)-ACN]; B %: 49%-79%, 10 min) to afford methyl 2,2-dichloro-3-(2-fluorophenyl)bicyclo[1.1.1]pentane-1-carboxylate (400 mg, 1.33 mmol, 27% yield) as yellow oil. ¹H NMR (400 MHz, DMSO-d₆) δ=7.53-7.39 (m, 2H), 7.33-7.21 (m, 2H), 3.76 (s, 3H), 3.05 (s, 2H), 2.68 (s, 2H).

To a solution of methyl 2,2-dichloro-3-(2-fluorophenyl)bicyclo[1.1.1]pentane-1-carboxylate (0.300 g, 1.04 mmol, 1.00 eq) in toluene (3.00 mL) were added (E)-1,1′-(diazene-1,2-diyl)dicyclohexanecarbonitrile (12.6 mg, 51.8 umol, 0.0500 eq) and tris(trimethylsilyl)silane (1.48 g, 5.95 mmol, 1.83 mL, 5.73 eq), the mixture was stirred at 80° C. for 2 h, (E)-1,1′-(diazene-1,2-diyl)dicyclohexanecarbonitrile (304 mg, 1.25 mmol, 1.20 eq) was added portionwise over 3 h. The reaction was stirred at 110° C. for another 216 h. The mixture was concentrated to give a residue. The residue was purified by flash silica gel chromatography (ISCO®; 40 g SepaFlash® Silica Flash Column, Eluent of 0˜100% ethyl acetate/petroleum ether @ 40 mL/min) to afford methyl 3-(2-fluorophenyl)bicyclo[1.1.1]pentane-1-carboxylate (0.4 g, crude) as yellow oil which was used to next step without further purification. ¹H NMR (400 MHz, DMSO-d₆) δ=7.35-7.28 (m, 1H), 7.26-7.19 (m, 1H), 7.18-7.10 (m, 2H), 3.64 (s, 3H), 2.34 (s, 6H).

To a mixture of 3-(2-fluorophenyl)bicyclo[1.1.1]pentane-1-carboxylate (0.300 g, 1.36 mmol, 1.00 eq) in methanol (3.00 mL) was added lithium hydroxide (114 mg, 2.72 mmol, 2.00 eq), the reaction was stirred at 25° C. for 3 h. The reaction was concentrated to give a residue. The residue was purified by reversed-phase HPLC (column: Shim-pack C18 150*25*10 um; mobile phase: [water (0.1% formic acid)-acetonitrile]) to afford 3-(2-fluorophenyl)bicyclo[1.1.1]pentane-1-carboxylic acid (0.150 g, 727 umol, 53% yield) as a yellow solid. ¹H NMR (400 MHz, DMSO-d₆) δ=12.78-12.12 (m, 1H), 7.30 (ddt, J=2.0, 5.4, 7.8 Hz, 1H), 7.22 (dt, J=2.0, 7.8 Hz, 1H), 7.18-7.08 (m, 2H), 2.29 (s, 6H).

To a mixture of 3-(2-fluorophenyl)bicyclo[1.1.1]pentane-1-carboxylic acid (0.0400 g, 193 umol, 1.00 eq) in dioxane (2.00 mL) was added diphenylphosphoryl azide (106 mg, 387 umol, 84.0 uL, 2.00 eq) and triethylamine (58.8 mg, 581 umol, 81.0 uL, 3.00 eq) under nitrogen atmosphere, the mixture was stirred at 25° C. for 1 h. Then 3-(6-(hydroxymethyl)-1-oxoisoindolin-2-yl)piperidine-2,6-dione 1 (58.5 mg, 213 umol, 1.10 eq) was added, the reaction mixture was stirred at 100° C. for another 2 h. The reaction was concentrated to give a residue. The residue was purified by Prep-HPLC (column: Phenomenex luna C18 150*25 mm*10 um; mobile phase: [water (0.225% FA)-ACN]; B %: 40%-70%, 10 min) to afford (2-(2,6-dioxopiperidin-3-yl)-3-oxoisoindolin-5-yl)methyl (3-(2-fluorophenyl)bicyclo[1.1.1]pentan-1-yl) carbamate 374 (41.5 mg, 85.1 umol, 43% yield, 98% purity) as a white solid. ¹H NMR (400 MHz, DMSO-d₆) δ=11.00 (br d, J=2.4 Hz, 1H), 8.17 (br s, 1H), 7.72 (s, 1H), 7.62 (s, 2H), 7.32-7.24 (m, 1H), 7.24-7.17 (m, 1H), 7.16-7.07 (m, 2H), 5.19-5.07 (m, 3H), 4.49-4.42 (m, 1H), 4.38-4.28 (m, 1H), 2.98-2.85 (m, 1H), 2.60 (br dd, J=2.2, 15.2 Hz, 1H), 2.40 (br dd, J=4.2, 13.2 Hz, 1H), 2.27 (s, 6H), 2.04-1.97 (m, 1H). MS (ESI) m/z 478.2 [M+H]⁺

Compounds 375 and 376: The (2-(2,6-dioxopiperidin-3-yl)-3-oxoisoindolin-5-yl)methyl (3-chloro-4-methylphenyl)carbamate (990 mg, 2.24 mmol) was purified by SFC (column: DAICEL CHIRALPAK IC (250 mm*30 mm, 10 um); mobile phase: [isopropanol-acetonitrile]; B %: 60%-60%, 5.2; 250 min) and lyophilized to afford (R)-(2-(2,6-dioxopiperidin-3-yl)-3-oxoisoindolin-5-yl)methyl(3-chloro-4-methylphenyl)carbamate 375 (353 mg, 791 umol, 35% yield, 99% purity) as a white solid and (S)-(2-(2,6-dioxopiperidin-3-yl)-3-oxoisoindolin-5-yl)methyl(3-chloro-4-methylphenyl)carbamate 376 (344 mg, 771 umol, 34% yield, 99% purity) as a white solid. (The absolute configuration was assigned arbitrarily)

375: ¹H NMR (400 MHz, DMSO-d₆) δ=11.00 (br s, 1H), 9.93 (br s, 1H), 7.80 (s, 1H), 7.72-7.67 (m, 1H), 7.66-7.62 (m, 1H), 7.60 (s, 1H), 7.33-7.28 (m, 1H), 7.27-7.23 (m, 1H), 5.28 (s, 2H), 5.13 (dd, J=5.2, 13.2 Hz, 1H), 4.55-4.43 (m, 1H), 4.39-4.30 (m, 1H), 2.92 (ddd, J=5.2, 13.6, 17.6 Hz, 1H), 2.61 (td, J=2.0, 15.2 Hz, 1H), 2.47-2.35 (m, 1H), 2.26 (s, 3H), 2.07-1.98 (m, 1H). MS (ESI) m/z 442.0 [M+H]⁺.

376: ¹H NMR (400 MHz, DMSO-d₆) δ=11.01 (s, 1H), 9.94 (s, 1H), 7.80 (s, 1H), 7.71-7.67 (m, 1H), 7.66-7.62 (m, 1H), 7.60 (s, 1H), 7.32-7.28 (m, 1H), 7.27-7.23 (m, 1H), 5.28 (s, 2H), 5.13 (dd, J=5.2, 13.2 Hz, 1H), 4.51-4.44 (m, 1H), 4.39-4.31 (m, 1H), 2.98-2.86 (m, 1H), 2.60 (td, J=2.0, 17.2 Hz, 1H), 2.46-2.36 (m, 1H), 2.25 (s, 3H), 2.06-1.97 (m, 1H). MS (ESI) m/z 442.0 [M+H]⁺.

Compound 377: A mixture of 2-chloro-6-methyl-4-nitroaniline (3.00 g, 16.1 mmol, 1.00 eq), cuprous bromide (2.77 g, 19.3 mmol, 587 uL, 1.20 eq) in acetonitrile (15.0 mL) was stirred for 5 min at 0° C., then tert-butylnitrite (2.49 g, 24.1 mmol, 2.87 mL, 1.50 eq) was dissolved in acetonitrile (15.0 mL) was added dropwise to the mixture. The mixture was stirred at 20° C. for 36 h. The reaction mixture was concentrated under reduced pressure to give a residue, and purified by column chromatography on silica gel (petroleum ether/ethyl acetate=1/0) to give 2-bromo-1-chloro-3-methyl-5-nitrobenzene (2.88 g, 11.5 mmol, 71% yield) as a white solid. ¹H NMR (400 MHz, DMSO-d₆) δ=8.28 (d, J=2.6 Hz, 1H), 8.23 (d, J=2.6 Hz, 1H), 2.55 (s, 3H).

A mixture of 2-bromo-1-chloro-3-methyl-5-nitrobenzene (2.70 g, 10.8 mmol, 1.00 eq), iron powder (1.81 g, 32.3 mmol, 3.00 eq) and ammonium chloride (2.88 g, 53.9 mmol, 5.00 eq) in methanol (30.0 mL) and water (10.0 mL) was stirred at 80° C. for 2 h. The mixture was filtered and the filtrate was concentrated under reduced pressure. The mixture was diluted with sodium hydrogencarbonate (150 mL) and extracted with ethyl acetate (3×50.0 mL). The organic layer was dried over anhydrous sodium sulfate, filtered and concentrated in vacuum to give 4-bromo-3-chloro-5-methylaniline (2.40 g, crude) as a yellow solid. ¹H NMR (400 MHz, DMSO-d₆) δ=6.61 (s, 1H), 6.48 (d, J=2.5 Hz, 1H), 5.45 (s, 2H), 2.24 (s, 3H).

To a solution of 4-bromo-3-chloro-5-methylaniline (1.00 g, 4.54 mmol, 1.00 eq) and zine cyanide (799 mg, 6.80 mmol, 432 uL, 1.50 eq) in dimethyl formamide (20.0 mL) was added tetrakis[triphenylphosphine] palladium(0) (524 mg, 454 umol, 0.100 eq) under nitrogen. The mixture was stirred at 150° C. for 1 h under microwave. The mixture was filtered, the filtrate was diluted with water (50.0 mL) and extracted with ethyl acetate (3×30.0 mL). The combined organic layer was washed with brine (20.0 mL) and dried over anhydrous sodium sulfate, filtered and concentrated to give crude product, the filter cake and the aqueous phase was quenched by sodium hydroxide to pH>11 then dip in sodium hypochlorite overnight. The crude product was purified by reversed-phase HPLC (column: spherical C18, 20-45 um, 100 Å, SW 80, mobile phase: [water (0.1% Formic Acid)-ACN]) and lyophilized to give 4-amino-2-chloro-6-methylbenzonitrile (380 mg, 2.28 mmol, 50% yield) as a white solid. ¹H NMR (400 MHz, DMSO-d₆) δ=6.56 (d, J=1.6 Hz, 1H), 6.45-6.42 (m, 1H), 6.38 (s, 2H), 2.30 (s, 3H). MS (ESI) m/z 167.1 [M+H]⁺

To a solution of 4-amino-2-chloro-6-methylbenzonitrile (380 mg, 2.28 mmol, 1.00 eq) and pyridine (541 mg, 6.84 mmol, 552 uL, 3.00 eq) in acetonitrile (5.00 mL) was added phenyl carbonochloridate (393 mg, 2.51 mmol, 314 uL, 1.10 eq) dropwise at 0° C. The mixture was stirred at 25° C. for 2 h. The reaction mixture was concentrated under reduced pressure to give crude product. The crude product was purified by reversed-phase HPLC (column: spherical C18, 20-45 um, 100 Å, SW 40, mobile phase: [water (0.1% Formic Acid)-ACN]) and lyophilized to give phenyl (3-chloro-4-cyano-5-methylphenyl)carbamate (340 mg, 1.17 mmol, 51% yield, 99% purity) as a black solid. MS (ESI) m/z 287.0 [M+H]⁺

To a solution of phenyl (3-chloro-4-cyano-5-methylphenyl)carbamate (100 mg, 350. umol, 1.20 eq) and 3-(6-(hydroxymethyl)-1-oxoisoindolin-2-yl)piperidine-2,6-dione I (80.0 mg, 297 umol, 1.00 eq) in dimethyl formamide (2.00 mL) was added sodium hydride (23.3 mg, 583 umol, 60% purity, 2.00 eq) in portions at 0° C. The mixture was stirred at 20° C. for 0.5 h. The mixture was quenched by 1 M hydrochloric acid (1.00 mL) and filtered. The filtrate was purified by prep-HPLC (column: 3_Phenomenex Luna C18 75*30 mm*3 um; mobile phase: [water (0.05% HCl)-ACN]; B %: 41%-51%, 6 min) to give (2-(2,6-dioxopiperidin-3-yl)-3-oxoisoindolin-5-yl)methyl(3-chloro-4-cyano-5-methylphenyl)carbamate 377 (19.7 mg, 42.0 umol, 14% yield, 99% purity) as a white solid. ¹H NMR (400 MHz, DMSO-d₆) δ=10.65-10.31 (m, 1H), 8.41 (br s, 1H), 7.80 (s, 1H), 7.72-7.67 (m, 1H), 7.67-7.60 (m, 2H), 7.46 (s, 1H), 5.31 (s, 2H), 5.12 (br dd, J=4.7, 13.5 Hz, 1H), 4.51-4.43 (m, 1H), 4.38-4.30 (m, 1H), 2.95-2.87 (m, 1H), 2.62 (br s, 1H), 2.45 (s, 3H), 2.39 (br s, 1H), 2.04-1.97 (m, 1H). MS (ESI) m/z 467.1 [M+H]⁺

Compound 378: To a solution of 3-chloro-2,6-difluoroaniline (500 mg, 3.06 mmol, 1.00 eq) and pyridine (726 mg, 9.17 mmol, 740 uL, 3.00 eq) in acetonitrile (5.00 mL) was added phenyl carbonochloridate (526 mg, 3.36 mmol, 421 uL, 1.10 eq) at 0° C. The mixture was stirred at 20° C. for 1 h. The reaction mixture was concentrated under reduced pressure to give a residue. The residue was purified by reversed-phase HPLC (C18, 80 g; condition: 0.1% formic acid/acetonitrile) to give phenyl (3-chloro-2,6-difluorophenyl)carbamate (297 mg, 1.04 mmol, 34% yield, 99% purity) as a white solid. ¹H NMR (400 MHz, CDCl₃) δ=7.45-7.39 (m, 2H), 7.38-7.27 (m, 2H), 7.26-7.21 (m, 2H), 6.99 (dt, J=2.0, 9.0 Hz, 1H), 6.55 (br s, 1H). MS (ESI) m/z 283.9 [M+H]⁺

To a solution of phenyl (3-chloro-2,6-difluorophenyl)carbamate (91.0 mg, 321 umol, 1.10 eq) and 3-(6-(hydroxymethyl)-1-oxoisoindolin-2-yl)piperidine-2,6-dione 1 (80.0 mg, 292 umol, 1.00 eq) in dimethyl formamide (1.50 mL) was added sodium hydride (23.3 mg, 583 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex luna C18 150*25 mm*10 um; mobile phase: [water (0.2% FA)-ACN]; B %: 30%-50%, 10 min) and lyophilized to afford (2-(2,6-dioxopiperidin-3-yl)-3-oxoisoindolin-5-yl)methyl (3-chloro-2,6-difluorophenyl)carbamate 378 (72.5 mg, 155 umol, 53% yield, 99% purity) as a white solid. ¹H NMR (400 MHz, DMSO-d₆) δ=10.99 (br s, 1H), 9.78-9.43 (m, 1H), 7.76 (s, 1H), 7.64 (s, 2H), 7.57 (dt, J=5.5, 8.7 Hz, 1H), 7.26 (dt, J=1.8, 9.3 Hz, 1H), 5.27 (s, 2H), 5.12 (dd, J=5.0, 13.3 Hz, 1H), 4.52-4.43 (m, 1H), 4.42-4.24 (m, 1H), 2.95-2.87 (m, 1H), 2.61 (td, J=2.0, 15.4 Hz, 1H), 2.40 (br dd, J=4.3, 13.1 Hz, 1H), 2.05-1.97 (m, 1H). MS (ESI) m/z 464.2 [M+H]⁺

Compound 379: To a solution of 3-chloro-2-fluoroaniline (2.00 g, 13.7 mmol, 1.00 eq) in acetonitrile (30.0 mL) was added N-bromosuccinimide (2.20 g, 12.3 mmol, 0.900 eq). The mixture was stirred at 25° C. for 12 h. The reaction mixture was concentrated under reduced pressure to give oil. The residue was purified by column chromatography (petroleum ether/ethyl acetate=8/1 to 3/1) to give 4-bromo-3-chloro-2-fluoroaniline (3.00 g, 13.3 mmol, 97% yield) as a black oil. ¹H NMR (400 MHz, DMSO-d₆) δ=7.22 (dd, J=1.7, 8.8 Hz, 1H), 6.72-6.66 (m, 1H), 5.65 (s, 2H).

To a solution of 4-bromo-3-chloro-2-fluoroaniline (1.00 g, 4.46 mmol, 1.00 eq) in water (2.00 mL) and dioxane (10.0 mL) was added potassium trifluoro(methyl)borate (2.17 g, 17.8 mmol, 4.00 eq), potassium carbonate (1.85 g, 13.3 mmol, 3.00 eq) and [1,1′-bis(diphenylphosphino)ferrocene] dichloropalladium(II) (325 mg, 445 umol, 0.100 eq) under nitrogen. The mixture was stirred at 110° C. for 12 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by column chromatography (petroleum ether/ethyl acetate=8/1 to 5/1) to give 3-chloro-2-fluoro-4-methylaniline (0.700 g, 4.39 mmol, 98% yield) as a yellow solid. ¹H NMR (400 MHz, DMSO-d₆) δ=6.83 (d, J=7.8 Hz, 1H), 6.63 (t, J=8.5 Hz, 1H), 5.19 (s, 2H), 2.18 (s, 3H).

To a solution of 3-chloro-2-fluoro-4-methylaniline (0.700 g, 4.39 mmol, 1.00 eq) in acetonitrile (10.0 mL) was added phenyl carbonochloridate (824 mg, 5.26 mmol, 659 uL, 1.20 eq) and pyridine (1.73 g, 21.9 mmol, 1.77 mL, 5.00 eq). The mixture was stirred at 25° C. for 0.5 h. The reaction mixture was concentrated under reduced pressure to give a residue. The crude product was purified by reversed-phase HPLC (0.1% FA condition) to give phenyl (3-chloro-2-fluoro-4-methylphenyl)carbamate (0.900 g, 3.22 mmol, 73% yield) as a yellow solid. ¹H NMR (400 MHz, DMSO-d₆) δ=10.05 (br s, 1H), 7.53 (brt, J=7.9 Hz, 1H), 7.46-7.40 (m, 2H), 7.29-7.14 (m, 4H), 2.34 (s, 3H). MS (ESI) m/z 280.0 [M+H]⁺

To a solution of phenyl phenyl (3-chloro-2-fluoro-4-methylphenyl)carbamate (97.9 mg, 350 umol, 1.20 eq) in dimethyl formamide (2.00 mL) was added 3-(6-(hydroxymethyl)-1-oxoisoindolin-2-yl)piperidine-2,6-dione (80.0 mg, 291 umol, 1.00 eq) and sodium hydride (23.3 mg, 583 umol, 60% purity, 2.00 eq) at 0° C. The mixture was stirred at 0° C. for 0.5 h. The reaction mixture was quenched with formic acid (1.00 ml) to give a solution. The crude product was dissolved in dimethyl formamide (2.00 mL) and purified by prep-HPLC (column: Waters Xbridge 150*25 mm*5 um; mobile phase: [water (0.05% ammonia hydroxide v/v)-ACN]; B %: 15%-45%, 9 min) and lyophilized to give (2-(2,6-dioxopiperidin-3-yl)-3-oxoisoindolin-5-yl) methyl (3-chloro-2-fluoro-4-methylphenyl)carbamate 379 (79.1 mg, 172 umol, 59% yield) as a white solid. ¹H NMR (400 MHz, DMSO-d₆) δ=11.00 (br s, 1H), 9.64 (br s, 1H), 7.81 (s, 1H), 7.71-7.61 (m, 2H), 7.51 (brt, J=7.9 Hz, 1H), 7.16 (brd, J=8.3 Hz, 1H), 5.28 (s, 2H), 5.14 (dd, J=5.1, 13.2 Hz, 1H), 4.51-4.44 (m, 1H), 4.39-4.32 (m, 1H), 2.98-2.87 (m, 1H), 2.64-2.58 (m, 1H), 2.41 (br dd, J=4.6, 13.1 Hz, 1H), 2.33 (s, 3H), 2.07-1.99 (m, 1H). MS (ESI) m/z 460.0 [M+H]⁺

Compound 380: To a solution of 2-fluoro-3-methylaniline (500 mg, 4.00 mmol, 1.00 eq) in dimethyl formamide (10.0 mL) was added 1-chloropyrrolidine-2,5-dione (533 mg, 4.00 mmol, 1.00 eq). The mixture was stirred at 25° C. for 12 h. The reaction mixture was quenched by addition water (50.0 mL) and extracted with ethyl acetate (3×50.0 mL). The combined organic layers were washed with water (2×100 mL), dried over anhydrous sodium sulfate, filtered and concentrated under reduced pressure to give a residue. The residue was purified by flash silica gel chromatography (ISCO®; 80 g SepaFlash® Silica Flash Column, Eluent of 0˜50% Ethyl acetate/Petroleum ether gradient @ 80 mL/min) to give 4-chloro-2-fluoro-3-methylaniline (300 mg, 1.75 mmol, 22% yield, 93% purity) as purple oil. ¹H NMR (400 MHz, CDCl₃) δ=6.94 (dd, J=1.6, 8.5 Hz, 1H), 6.56 (t, J=8.9 Hz, 1H), 3.96-3.42 (m, 2H), 2.05 (s, 3H). MS (ESI) m/z 160.1 [M+H]⁺

To a mixture of 4-chloro-2-fluoro-3-methylaniline (300 mg, 1.88 mmol, 1.00 eq), pyridine (446 mg, 5.64 mmol, 455 uL, 3.00 eq) in acetonitrile (5.00 mL) was added phenyl carbonochloridate (324 mg, 2.07 mmol, 259 uL, 1.10 eq) at 0° C. Then the mixture was stirred at 20° C. for 1 h. The reaction mixture was concentrated under reduced pressure to give a residue. The residue was purified by reversed-phase HPLC (C18, 80 g; condition: water/acetonitrile=1/0 to 0/1, 0.1% formic acid) to give phenyl (4-chloro-2-fluoro-3-methylphenyl)carbamate (294 mg, 1.04 mmol, 55% yield, 99% purity) as a pink solid. ¹H NMR (400 MHz, DMSO-d₆) δ=10.04 (br s, 1H), 7.59 (br t, J=8.4 Hz, 1H), 7.45-7.40 (m, 2H), 7.30-7.21 (m, 4H), 2.28 (d, J=2.3 Hz, 3H). MS (ESI) m/z 280.1 [M+H]⁺

To a solution of phenyl (4-chloro-2-fluoro-3-methylphenyl)carbamate (112 mg, 401 umol, 1.10 eq) and 3-(6-(hydroxymethyl)-1-oxoisoindolin-2-yl)piperidine-2,6-dione 1 (100 mg, 365 umol, 1.00 eq) in dimethyl formamide (1.50 mL) was added sodium hydride (29.2 mg, 729 umol, 60% purity, 2.00 eq) at 0° C. The mixture was stirred at 0° C. for 1 h. The pH of the mixture was adjusted to around 6 by adding hydrochloric acid (1 M). The mixture was extracted with ethyl acetate (3×10 mL). The combined organic layers were dried over anhydrous sodium sulfate, filtered and concentrated under reduced pressure to give a residue. The residue was purified by prep-HPLC (column: Phenomenex luna C18 150*25 mm*10 um; mobile phase: [water (0.2% FA)-ACN]; B %: 42%-62%, 10 min) and lyophilized to afford (2-(2,6-dioxopiperidin-3-yl)-3-oxoisoindolin-5-yl)methyl(4-chloro-2-fluoro-3-methylphenyl)carbamate 380 (97.2 mg, 207 umol, 57% yield, 98% purity) as a white solid. ¹H NMR (400 MHz, DMSO-d₆) δ=10.98 (br s, 1H), 9.61 (br s, 1H), 7.80 (s, 1H), 7.69-7.61 (m, 2H), 7.56 (brt, J=8.7 Hz, 1H), 7.25 (dd, J=1.3, 8.8 Hz, 1H), 5.28 (s, 2H), 5.13 (dd, J=5.1, 13.3 Hz, 1H), 4.51-4.43 (m, 1H), 4.38-4.30 (m, 1H), 2.97-2.87 (m, 1H), 2.64-2.57 (m, 1H), 2.40 (br dd, J=4.4, 13.2 Hz, 1H), 2.25 (d, J=2.4 Hz, 3H), 2.05-1.97 (m, 1H). MS (ESI) m/z 459.9 [M+H]⁺

Compound 381: To a solution of 5-bromo-2-nitropyridine (2.00 g, 9.85 mmol, 1.00 eq) in toluene (25.0 mL) was added cyclobutylboronic acid (1.48 g, 14.8 mmol, 1.50 eq), cesium carbonate (4.82 g, 14.8 mmol, 1.50 eq) and [1,1′-bis(diphenylphosphino)ferrocene]dichloropalladium(II) (577 mg, 788 umol, 0.0800 eq) under nitrogen atmosphere. The mixture was stirred at 100° C. for 12 h. The residue was diluted with water (200 mL) and extracted with ethyl acetate (3×40.0 mL). The combined organic layers were washed with brine (30.0 mL), dried over sodium sulfate, filtered and concentrated under reduced pressure to give a residue. The residue was purified by column chromatography on silica gel (Petroleum ether/Ethyl acetate=1/0 to Petroleum ether/Ethyl acetate=5/1) to afford 5-cyclobutyl-2-nitropyridine (650 mg, 3.61 mmol, 36% yield) as a white solid. ¹H NMR (400 MHz, DMSO-d₆) δ=8.54 (d, J=2.4 Hz, 1H), 8.26 (d, J=8.4 Hz, 1H), 8.11 (dd, J=2.3, 8.4 Hz, 1H), 3.73 (quin, J=8.8 Hz, 1H), 2.37 (tq, J=2.6, 8.4 Hz, 2H), 2.27-2.12 (m, 2H), 2.12-2.00 (m, 1H), 1.93-1.82 (m, 1H). MS (ESI) m/z 179.1 [M+H]⁺.

To a solution of 5-cyclobutyl-2-nitropyridine (600 mg, 3.37 mmol, 1.00 eq) in ethyl acetate (20.0 mL) was added palladium on carbon (3.37 mmol, 10% purity, 1.00 eq) under hydrogen atmosphere. The mixture was stirred at 25° C. for 2 h. The reaction mixture was filtered and the filtrate was concentrated under reduced pressure to afford 5-cyclobutylpyridin-2-amine (480 mg, 3.24 mmol, 96% yield) as a white solid. ¹H NMR (400 MHz, DMSO-d₆) δ=7.74 (d, J=2.4 Hz, 1H), 7.30 (dd, J=2.4, 8.4 Hz, 1H), 6.40 (d, J=8.4 Hz, 1H), 5.66 (s, 2H), 3.34-3.28 (m, 1H), 2.19 (tq, J=2.3, 8.0 Hz, 2H), 2.03-1.93 (m, 2H), 1.91-1.72 (m, 2H).

To a solution of 5-cyclobutylpyridin-2-amine (300 mg, 2.02 mmol, 1.00 eq) in acetonitrile (3.00 mL) were added pyridine (480 mg, 6.07 mmol, 490 uL, 3.00 eq) and phenyl carbonochloridate (349 mg, 2.23 mmol, 279 uL, 1.10 eq). The mixture was stirred at 25° C. for 2 h. The reaction mixture was concentrated under reduced pressure to afford a residue. The residue was triturated with dimethyl formamide (5.00 mL) at 25° C. for 5 min to afford phenyl (5-cyclobutylpyridin-2-yl)carbamate (100 mg, 365 umol, 18% yield) as a white solid. MS (ESI) m/z 269.2 [M+H]⁺

To a solution of phenyl (5-cyclobutylpyridin-2-yl)carbamate (40.0 mg, 149 umol, 1.00 eq) in dimethyl formamide (500 uL) was added sodium hydride (11.9 mg, 298 umol, 60% purity, 2.00 eq) and 3-(6-(hydroxymethyl)-1-oxoisoindolin-2-yl)piperidine-2,6-dione I (45.0 mg, 164 umol, 1.10 eq). The mixture was stirred at 25° C. for 1 h. The pH of the mixture was adjusted to 7 with formic acid. Then the mixture was filtered and the filter cake was obtained as a crude product. The crude product was triturated with dimethyl formamide (5.00 mL) at 25° C. for 5 min to afford (2-(2,6-dioxopiperidin-3-yl)-3-oxoisoindolin-5-yl)methyl (5-cyclobutylpyridin-2-yl)carbamate 381 (49.77 mg, 99.6 umol, 67% yield, 99% purity, formic acid) as a white solid. ¹H NMR (400 MHz, DMSO-d₆) δ=11.13-10.84 (m, 1H), 10.25 (s, 1H), 8.12 (s, 1H), 7.82-7.73 (m, 2H), 7.71-7.58 (m, 3H), 5.28 (s, 2H), 5.12 (br dd, J=5.1, 13.2 Hz, 1H), 4.52-4.41 (m, 1H), 4.39-4.29 (m, 1H), 3.51-3.46 (m, 1H), 2.98-2.83 (m, 1H), 2.63-2.57 (m, 1H), 2.40 (br dd, J=4.3, 13.2 Hz, 1H), 2.27 (q, J=8.4 Hz, 2H), 2.14-2.05 (m, 2H), 2.04-1.90 (m, 2H), 1.88-1.75 (m, 1H). MS (ESI) m/z 449.3 [M+H]⁺

Compound 382: To a solution of 7-bromo-5-methyl-benzofuran (200 mg, 947 umol, 1.00 eq), diphenylmethanimine (206 mg, 1.14 mmol, 190 uL, 1.20 eq), diphenyl phosphine (118 mg, 189 umol, 0.200 eq) and sodium tert-butoxide (136 mg, 1.42 mmol, 1.50 eq) in toluene (3.00 mL) was added tris(dibenzylideneacetone)dipalladium(0) (86.8 mg, 94.8 umol, 0.100 eq), the mixture was stirred at 110° C. for 12 h. The reaction mixture was poured into water (80.0 mL) and stirred for 10 min. The aqueous phase was extracted with ethyl acetate (3×40.0 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=1/0 to 5/1) to afford N-(diphenylmethylene)-5-methylbenzofuran-7-amine (350 mg, crude) as a yellow oil. ¹H NMR (400 MHz, CDCl₃) δ=7.92-7.79 (m, 3H), 7.55-7.42 (m, 5H), 7.27-7.15 (m, 5H), 6.61 (d, J=2.1 Hz, 1H), 2.29 (s, 3H).

To a solution of N-(diphenylmethylene)-5-methylbenzofuran-7-amine (350 mg, 1.12 mmol, 1.00 eq) in tetrahydrofuran (6.00 mL) was added hydrochloric acid (5 M, 1.00 mL, 4.45 eq), the mixture was stirred at 25° C. for 1 h. The reaction mixture was adjust pH=8-9 with 4 M sodium hydroxide (2.00 mL) and then poured into water (60.0 mL). The aqueous phase was extracted with ethyl acetate (3×40.0 mL). The combined organic phase was washed with brine (80.0 mL), dried over anhydrous sodium sulfate, filtered and concentrated in vacuum to give a residue. The residue was purified by silica gel chromatography (Petroleum ether/Ethyl acetate=1/0 to 10/1) to afford 5-methylbenzofuran-7-amine (100 mg, 679 umol, 60% yield) as a yellow oil. ¹H NMR (400 MHz, CDCl₃) δ=7.57 (d, J=2.2 Hz, 1H), 6.85 (s, 1H), 6.68 (d, J=2.1 Hz, 1H), 6.52 (d, J=0.6 Hz, 1H), 4.03-3.62 (m, 2H), 2.39 (s, 3H).

To a solution of 5-methylbenzofuran-7-amine (100 mg, 679 umol, 1.00 eq) in methanol (5.00 mL) was added palladium/carbon (20.0 mg, 10% purity), the mixture was stirred at 25° C. for 1 h under hydrogen atmosphere. The reaction mixture was filtered to give a filtrate, the filtrate was concentrated to afford 5-methyl-2,3-dihydrobenzofuran-7-amine (70.0 mg, 469 umol, 69% yield) as a yellow oil. ¹H NMR (400 MHz, CDCl₃) δ=6.40 (s, 1H), 6.31 (s, 1H), 4.47 (t, J=8.7 Hz, 2H), 3.10-3.03 (m, 2H), 2.14 (s, 3H).

To a solution of 5-methyl-2,3-dihydrobenzofuran-7-amine (70.0 mg, 469 umol, 1.00 eq) and pyridine (111 mg, 1.41 mmol, 113 uL, 3.00 eq) in acetonitrile (2.00 mL) was added phenyl carbonochloridate (88.2 mg, 563 umol, 70.5 uL, 1.20 eq), the mixture was stirred at 25° C. for 1 h. The reaction mixture was concentrated to give a residue. The residue was purified by reversed-phase HPLC (column: spherical C18, 20-45 um, 40 A, SW 120, mobile phase: [water (0.1% Formic Acid)-acetonitrile) to afford phenyl (5-methyl-2,3-dihydrobenzofuran-7-yl)carbamate (100 mg, 371 umol, 79% yield) as a white solid.

To a solution of phenyl (5-methyl-2,3-dihydrobenzofuran-7-yl)carbamate (100 mg, 371 umol, 1.20 eq) and 3-(6-(hydroxymethyl)-1-oxoisoindolin-2-yl)piperidine-2,6-dione I (84.9 mg, 309 umol, 1.00 eq) in dimethylformamide (1.00 mL) was added sodium hydride (20.0 mg, 500 umol, 60% purity, 1.62 eq), the mixture was stirred at 25° C. for 1 h. The reaction mixture was quenched with 1 M hydrochloric acid (0.500 mL) and filtered to give a filtrate. The filtrate was purified by Prep-HPLC (column: Phenomenex luna C18 150*25 mm*10 um; mobile phase: [water (0.225% FA)-ACN]; B %: 29%-59%, 10 min) to afford (2-(2,6-dioxopiperidin-3-yl)-3-oxoisoindolin-5-yl)methyl(5-methyl-2,3-dihydrobenzofuran-7-yl)carbamate 382 (70.11 mg, 155 umol, 50% yield) as a white solid. ¹H NMR (400 MHz, DMSO-d₆) δ=10.99 (br s, 1H), 8.99 (br s, 1H), 7.79 (s, 1H), 7.70-7.59 (m, 2H), 7.08 (br s, 1H), 6.81 (s, 1H), 5.23 (s, 2H), 5.14 (br dd, J=5.1, 13.3 Hz, 1H), 4.59-4.43 (m, 3H), 4.40-4.29 (m, 1H), 3.15 (brt, J=8.6 Hz, 2H), 2.99-2.85 (m, 1H), 2.61 (br d, J=17.6 Hz, 1H), 2.45-2.34 (m, 1H), 2.20 (s, 3H), 2.08-1.98 (m, 1H). MS (ESI) m/z 450.1 [M+H]⁺

Compound 383: To a solution of 6-chloro-5-methylpyridin-2-amine (1.00 g, 7.01 mmol, 1.00 eq) in acetonitrile (10.0 mL) were added phenyl carbonochloridate (1.21 g, 7.71 mmol, 967 uL, 1.10 eq) and pyridine (1.66 g, 21.0 mmol, 1.70 mL, 3.00 eq). The mixture was stirred at 25° C. for 12 h. The mixture was added water (10 mL) and extracted with ethyl acetate (3×25 mL). The combined organic phases were washed with brine (10 mL), dried over anhydrous sodium sulfate, filtered and concentrated in vacuum to give a residue. The residue was purified by silica gel chromatography (petroleum ether/ethyl acetate=10/1 to 5/1) to afford phenyl (6-chloro-5-methylpyridin-2-yl)carbamate (0.400 g, 1.46 mmol, 21% yield, 96% purity) as a white solid. ¹H NMR (400 MHz, CDCl₃) δ=7.83 (d, J=8.4 Hz, 1H), 7.66 (br s, 1H), 7.58 (d, J=8.4 Hz, 1H), 7.44-7.39 (m, 2H), 7.27 (s, 1H), 7.22-7.16 (m, 2H), 2.35 (s, 3H).

To a solution of 3-(6-(hydroxymethyl)-1-oxoisoindolin-2-yl)piperidine-2,6-dione I (105 mg, 383 umol, 1.00 eq) in N,N′-dimethyl formamide (1.00 mL) were added phenyl (6-chloro-5-methylpyridin-2-yl)carbamate (121 mg, 460 umol, 1.20 eq) and sodium hydride (30.6 mg, 766 umol, 60% purity, 2.00 eq) at 0° C. The mixture was stirred at 0° C. for 1 h. The pH of the mixture was adjusted to 7 with hydrochloric acid (1 M). The mixture was purified by Prep-HPLC (column: Phenomenex Synergi C18 150×25 mm×10 um; mobile phase: [water (0.225% formic acid)-acetonitrile]; B %: 31%-61%, 10 min) and lyophilized to afford (2-(2,6-dioxopiperidin-3-yl)-3-oxoisoindolin-5-yl)methyl (6-chloro-5-methylpyridin-2-yl)carbamate 383 (81.33 mg, 182 umol, 47% yield, 99% purity) as a white solid. ¹H NMR (400 MHz, DMSO-d₆) δ=10.99 (br s, 1H), 10.57 (s, 1H), 7.79 (s, 1H), 7.76 (br d, J=8.4 Hz, 1H), 7.74 (br d, J=8.0 Hz, 1H), 7.66 (dd, J=1.6 Hz, 8.0 Hz 1H), 7.63 (br d, J=8.0 Hz, 1H), 5.29 (s, 2H), 5.13 (dd, J=5.2, 13.2 Hz, 1H), 4.46 (br d, J=17.2 Hz, 1H), 4.34 (br d, J=17.6 Hz, 1H), 2.97-2.86 (m, 1H), 2.64-2.56 (m, 1H), 2.46-2.34 (m, 1H), 2.26 (s, 3H), 2.05-1.96 (m, 1H). MS (ESI) m/z 442.9 [M+H]⁺

Compounds 384 and 385: To a stirred solution of 3-(tert-butyl)cyclobutanone (450 mg, 3.57 mmol, 1.00 eq) in ethyl alcohol (27.0 mL) were added hydroxylamine (495 mg, 7.13 mmol, 2.00 eq, hydride acid) and sodium acetate (1.17 g, 14.2 mmol, 4.00 eq), then the resulting mixture was heated to 80° C. and stirred for 16 h. The reaction mixture was quenched by addition water (10.0 mL), and then extracted with ethyl acetate (3×20.0 mL). The combined organic layers were washed with brine (3×20.0 mL), dried over sodium sulfate, filtered and concentrated under reduced pressure to give 3-tert-butylcyclobutanone oxime (440 mg, 3.12 mmol, 87% yield) as a white solid. ¹H NMR (400 MHz, CDCl₃) δ=2.84-2.67 (m, 2H), 2.63-2.56 (m, 2H), 2.18 (tt, J=9.07, 7.44 Hz, 1H), 0.81 (s, 1H). MS (ESI) m/z 142.2 [M+H]⁺

To a stirred solution of 3-tert-butylcyclobutanone oxime (200 mg, 1.42 mmol, 1.00 eq) in concentrated hydrochloric acid (1.00 mL) and methanol (10.0 mL), palladium on carbon (100 mg, 10% purity) was then added. Then the resulting solution was degassed with nitrogen for three times and degassed with hydrogen for three times. The reaction mixture was stirred at 15° C. for 12 h. The reaction mixture was filtrated through diatomite, and the filter cake was washed with ethyl alcohol, the filtrate was concentrated under reduced pressure to give 3-(tert-butyl)cyclobutanamine (220 mg, crude, hydride acid) as a yellow solid. ¹H NMR (400 MHz, CDCl₃) δ=8.49-8.36 (m, 3H), 3.74 (br, 0.6H), 3.60 (br, 0.4H), 2.52 (br, 0.6H), 2.32-2.28 (m, 3H), 2.09-2.07 (m, 1H), 1.94-1.92 (m, 1H), 0.86 (d, J=4.38 Hz).

To a stirred solution of 3-(tert-butyl)cyclobutanamine (220 mg, 1.34 mmol, 1.00 eq, hydride acid) and phenyl carbonochloridate (231 mg, 1.48 mmol, 0.185 mL, 1.10 eq) in nitrile (10.0 mL) was added pyridine (318 mg, 4.03 mmol, 0.325 mL, 3.00 eq), then the mixture was stirred at 20° C. for 16 h. The reaction mixture was quenched by addition water (10.0 mL), and extracted with ethyl acetate (3×10.0 mL). The combined organic layers were washed with brine (3×10.0 mL), dried over sodium sulfate, filtered and concentrated under reduced pressure to give a residue. The residue was purified by flash silica gel chromatography (ISCO®; 10 g SepaFlash® Silica Flash Column, Eluent of 0˜30% Ethylacetate/Petroleum ethergradient @ 12 mL/min) to give phenyl (3-(tert-butyl)cyclobutyl)carbamate (220 mg, 0.890 mmol, 66% yield) as a white solid. ¹H NMR (400 MHz, CDCl₃) δ=7.41-7.13 (m, 5H), 5.25-5.06 (m, 1H), 4.18-4.08 (m, 0.5H), 4.0.6-3.96 (m, 0.4H), 2.34-2.25 (m, 2.5H), 2.01-1.97 (m, 1H), 1.89-1.80 (m, 0.5H), 1.68-1.60 (m, 1H), 0.87 (d, J=11.62 Hz). MS (ESI) m/z 248.2 [M+H]⁺

To a stirred solution of phenyl (3-(tert-butyl)cyclobutyl)carbamate (87.3 mg, 0.353 mmol, 1.10 eq) and 3-(6-(hydroxymethyl)-1-oxoisoindolin-2-yl)piperidine-2,6-dione 1 (110 mg, 0.321 mmol, 80% purity, 1.00 eq) in dimethylformamide (2.00 mL) was added sodium hydride (25.6 mg, 0.642 mmol, 60% purity, 2.00 eq) at 0° C., then the mixture was stirred at 20° C. for 1.5 h. The reaction mixture was quenched by addition the mixture to aqueous formic acid at 0° C., and then extracted with ethyl acetate (3×10.0 mL). The combined organic layers were washed with brine (3×10.0 mL), dried over sodium sulfate, filtered and concentrated under reduced pressure to give a residue. The residue was purified by prep-HPLC (formic acid condition: column: Unisil 3-100 C18 Ultra 150*50 mm*3 um; mobile phase: [water (0.225% formic acid)-acetonitrile]; B %: 40%-60%, 10 min) to give desired compound 384 cis-(2-(2,6-dioxopiperidin-3-yl)-3-oxoisoindolin-5-yl)methyl ((1 s,3s)-3-(tert-butyl)cyclobutyl)carbamate (42.2 mg, 98.6 umol, yield 30.74%) as a white solid, (formic acid condition: column: Phenomenex luna C18 150*25 mm*10 um; mobile phase: [water (0.225% formic acid)-acetonitrile]; B %: 37%-67%, 10 min) compound 385 trans-(2-(2,6-dioxopiperidin-3-yl)-3-oxoisoindolin-5-yl)methyl ((1 s,3s)-3-(tert-butyl)cyclobutyl)carbamate (26.4 mg, 61.7 umol, yield 19.25%) as an off-white solid.

384: ¹H NMR (400 MHz, DMSO-d₆) δ=10.99 (s, 1H), 7.71 (s, 1H), 7.66 (d, J=6.9 Hz, 1H), 7.60 (s, 2H), 5.15-5.12 (m, 3H), 4.49-4.44 (m, 1H), 4.35-4.31 (m, 1H), 3.87-3.82 (m, 1H), 2.97-2.88 (m, 1H), 2.63-2.58 (m, 1H), 2.43-2.35 (m, 1H), 2.10-1.98 (m, 4H), 1.94-1.87 (m, 2H), 0.81 (s, 9H). MS (ESI) m/z 428.2 [M+H]⁺

385: ¹H NMR (400 MHz, DMSO-d₆) δ=10.99 (s, 1H), 7.70 (s, 1H), 7.60 (s, 2H), 7.53 (d, J=7.88 Hz, 1H), 5.15-5.11 (m, 3H), 4.49-4.44 (m, 1H), 4.35-4.31 (m, 1H), 3.76-3.70 (m, 1H), 2.97-2.87 (m, 1H), 2.63-2.58 (m, 1H), 2.45-2.33 (m, 1H), 2.07-1.99 (m, 3H), 1.72-1.58 (m, 3H), 0.78 (s, 9H). MS (ESI) m/z 428.2 [M+H]⁺

Compound 386: A mixture of 1-bromo-2,3-dimethyl-5-nitrobenzene (400 mg, 1.74 mmol, 1.00 eq) in dimethyl formamide (5.00 mL) was added Zinc cyanide (204 mg, 1.74 mmol, 110 uL, 1.00 eq), tris(dibenzylideneacetone)dipalladium(0) (159 mg, 174 umol, 0.10 eq) and diphenylphosphoryl azide (193 mg, 348 umol, 0.20 eq). The mixture was stirred at 130° C. for 3 h under nitrogen atmosphere. The reaction mixture was filtered. The filtrate was diluted with water (30.0 mL) and extracted with ethyl acetate (3×30.0 mL). The combined organic layers were washed with brine (2×30.0 mL), dried over anhydrous sodium sulfate, filtered and concentrated under reduced pressure to give a residue. The residue was purified by column chromatography (SiO₂, petroleum ether/ethyl acetate=1/0 to 10/1) to give 2,3-dimethyl-5-nitrobenzonitrile (315 mg, crude) as a yellow solid. ¹H NMR (400 MHz, CDCl₃) δ=8.34 (d, J=2.3 Hz, 1H), 8.23 (d, J=2.3 Hz, 1H), 2.60 (s, 3H), 2.47 (s, 3H).

To a solution of 2,3-dimethyl-5-nitrobenzonitrile (300 mg, 1.70 mmol, 1.00 eq) in methanol (10.0 mL) and water (2.00 mL) was added ferrous powder (475 mg, 8.51 mmol, 5.00 eq) and saturated ammonium chloride (728 mg, 13.6 mmol, 8.00 eq). The mixture was stirred at 80° C. for 2 h. The reaction mixture was filtered and concentrated under reduced pressure to give a residue. The reaction mixture was diluted with aqueous saturated sodium bicarbonate (20.0 mL) and extracted with ethyl acetate (3×30.0 mL). The combined organic layers were washed with dried over anhydrous sodium sulfate, filtered and concentrated under reduced pressure to give 5-amino-2,3-dimethylbenzonitrile (150 mg, 821 umol, 48% yield, 80% purity) as a yellow solid. MS (ESI) m/z 147.2 [M+H]⁺

To a solution of 5-amino-2,3-dimethylbenzonitrile (150 mg, 1.03 mmol, 1.00 eq) and pyridine (243 mg, 3.08 mmol, 248 uL, 3.00 eq) in acetonitrile (5.00 mL) was added phenyl carbonochloridate (177 mg, 1.13 mmol, 141 uL, 1.10 eq) at 0° C. The mixture was stirred at 20° C. for 1 h. The reaction mixture was concentrated under reduced pressure to give a residue. The residue was purified by flash silica gel chromatography (ISCO®; 20 g SepaFlash® Silica Flash Column, Eluent of 0˜25% Ethylacetate/Petroleum ethergradient @60 mL/min) to give phenyl (3-cyano-4,5-dimethylphenyl)carbamate (150 mg, 552 umol, 54% yield, 98% purity) as an off-white solid. ¹H NMR (400 MHz, DMSO-d₆) δ=10.43 (br s, 1H), 7.68 (d, J=2.0 Hz, 1H), 7.56 (d, J=1.8 Hz, 1H), 7.46-7.41 (m, 2H), 7.29-7.22 (m, 3H), 2.35 (s, 3H), 2.27 (s, 3H). MS (ESI) m/z 267.1 [M+H]⁺

To a solution of phenyl phenyl (3-cyano-4,5-dimethylphenyl)carbamate (107 mg, 401 umol, 1.10 eq) and 3-(6-(hydroxymethyl)-1-oxoisoindolin-2-yl)piperidine-2,6-dione I (100 mg, 365 umol, 1.00 eq) in dimethyl formamide (1.50 mL) was added sodium hydride (29.2 mg, 729 umol, 60% purity, 2.00 eq) at 0° C. The mixture was stirred at 0° C. for 1 h. The pH of the mixture was adjusted to around 6 by adding hydrochloric acid. The mixture was extracted with ethyl acetate (3×10.0 mL). The combined organic layers were dried over anhydrous sodium sulfate, filtered and concentrated under reduced pressure to give a residue. The residue was purified by prep-HPLC (column: Phenomenex luna C18 150*25 mm*10 um; mobile phase: [water (0.2% FA)-ACN]; B %: 32%-62%, 10 min) and lyophilized to give (2-(2,6-dioxopiperidin-3-yl)-3-oxoisoindolin-5-yl)methyl(3-cyano-4,5-dimethylphenyl)carbamate 386 (71.81 mg, 144 umol, 85% yield, 99% purity, formate) as a white solid. ¹H NMR (400 MHz, DMSO-d₆) δ=10.99 (br s, 1H), 10.03 (s, 1H), 7.79 (s, 1H), 7.73-7.60 (m, 3H), 7.52 (s, 1H), 5.28 (s, 2H), 5.13 (dd, J=5.1, 13.2 Hz, 1H), 4.51-4.43 (m, 1H), 4.38-4.29 (m, 1H), 2.98-2.85 (m, 1H), 2.60 (td, J=2.0, 15.4 Hz, 1H), 2.40 (br dd, J=4.4, 13.1 Hz, 1H), 2.33 (s, 3H), 2.25 (s, 3H), 2.05-1.97 (m, 1H). MS (ESI) m/z 447.0 [M+H]⁺

Compound 387: A mixture of 5-fluoro-2,3-dihydrobenzofuran (0.500 g, 3.62 mmol, 1.00 eq) in nitric acid (5.00 mL) was stirred at −10° C. for 0.5 h. The mixture was quenched by addition water (5.00 mL) at −10° C. and extracted with ethyl acetate (3×10.0 mL). The combined organic layers were concentrated to afford 5-fluoro-7-nitro-2,3-dihydrobenzofuran (600 mg, 3.28 mmol, 90% yield) as a yellow solid. ¹H NMR (400 MHz, CDCl₃) δ=7.63 (dd, J=2.8, 8.8 Hz, 1H), 7.23 (tdd, J=1.2, 2.8, 7.2 Hz, 1H), 4.86 (s, 2H), 3.34 (dt, J=1.2, 8.8 Hz, 2H).

A solution of 5-fluoro-7-nitro-2,3-dihydrobenzofuran (600 mg, 3.28 mmol, 1.00 eq) in ethyl acetate (15.0 mL) was added palladium on carbon (500 mg, 10% purity), the reaction mixture was stirred at 25° C. for 12 h under hydrogen atmosphere. The reaction mixture was filtered and the filtrate was concentrated to afford 5-fluoro-2,3-dihydrobenzofuran-7-amine (400 mg, 2.61 mmol, 79% yield) as a red solid. ¹H NMR (400 MHz, DMSO-d₆) δ=6.23 (brd, J=8.8 Hz, 2H), 4.91 (s, 2H), 4.47 (t, J=8.8 Hz, 2H), 3.09 (t, J=8.8 Hz, 2H). MS (ESI) m/z 274.0 [M+H]⁺

To a mixture of 5-fluoro-2,3-dihydrobenzofuran-7-amine (0.400 g, 2.61 mmol, 1.00 eq) in acetonitrile (15.0 mL) were added phenyl carbonochloridate (429 mg, 2.74 mmol, 343 uL, 1.05 eq) and pyridine (413 mg, 5.22 mmol, 421 uL, 2.00 eq), the reaction mixture was stirred at 25° C. for 12 h. The reaction was concentrated to give a residue. The residue was purified by reversed-phase HPLC (column: Shim-pack C18 150*25*10 um; mobile phase: [water (0.1% formic acid)-acetonitrile]) to afford phenyl (5-fluoro-2,3-dihydrobenzofuran-7-yl)carbamate (0.500 g, 1.83 mmol, 70% yield) as a red solid. ¹H NMR (400 MHz, DMSO-d₆) δ=9.68 (br s, 1H), 7.45-7.39 (m, 2H), 7.29-7.16 (m, 4H), 6.90 (dd, J=2.6, 7.8 Hz, 1H), 4.60 (t, J=8.8 Hz, 2H), 3.23 (t, J=8.8 Hz, 2H).

To a mixture of 3-(6-(hydroxymethyl)-1-oxoisoindolin-2-yl)piperidine-2,6-dione 1 (80.0 mg, 291 umol, 1.00 eq) and phenyl (5-fluoro-2,3-dihydrobenzofuran-7-yl)carbamate (79.7 mg, 291 umol, 1.00 eq) in dimethylformamide (1.00 mL) was added sodium hydride (23.3 mg, 583 umol, 60% purity, 2.00 eq), the mixture was stirred at 25° C. for 1 h. The pH of the mixture was adjusted to 7 with formic acid, the mixture was diluted with dimethylformamide (1.00 mL). The mixture was purified by Prep-HPLC (column: Phenomenex Luna C18 150*25 mm*10 um; mobile phase: [water (0.225% formic acid)-acetonitrile]; B %: 26%-56%, 10 min) to afford (2-(2,6-dioxopiperidin-3-yl)-3-oxoisoindolin-5-yl)methyl(5-fluoro-2,3-dihydrobenzofuran-7-yl) carbamate 387 (68.1 mg, 148 umol, 50% yield, 99% purity) as a white solid. ¹H NMR (400 MHz, DMSO-d₆) δ=10.99 (br s, 1H), 9.32 (br s, 1H), 7.81 (s, 1H), 7.70-7.58 (m, 2H), 7.24 (br d, J=10.4 Hz, 1H), 6.85 (dd, J=2.4, 8.0 Hz, 1H), 5.25 (s, 2H), 5.13 (dd, J=5.2, 13.2 Hz, 1H), 4.56 (t, J=8.8 Hz, 2H), 4.50-4.43 (m, 1H), 4.37-4.30 (m, 1H), 3.20 (t, J=8.8 Hz, 2H), 2.91 (ddd, J=5.4, 13.2, 17.6 Hz, 1H), 2.64-2.57 (m, 1H), 2.45-2.35 (m, 1H), 2.05-1.97 (m, 1H). MS (ESI) m/z 453.9 [M+H]⁺

Compound 388: To a solution of 5-bromo-2-nitropyridine (5.00 g, 24.6 mmol, 1.00 eq), potassium trifluoro(prop-1-en-2-yl) borate (5.47 g, 36.9 mmol, 1.50 eq) and [1,1′-bis(diphenylphosphino)ferrocene] dichloropalladium(II) (901 mg, 1.23 mmol, 0.0500 eq) in dioxane (50.0 mL) and water (5.00 mL) was added potassium carbonate (10.2 g, 73.8 mmol, 3.00 eq), then evacuated with vacuum and back filled with nitrogen 3 times. The mixture was stirred at 80° C. for 2 h. The reaction mixture was poured into water (200 mL) and extracted with ethyl acetate (3×50.0 mL). The combined organic phase was separated, washed with brine (100 mL), dried over anhydrous sodium sulfate, filtered and concentrated to give a residue which was purified by silica gel chromatography (petroleum ether/ethyl acetate=20/1 to 10/1) to afford 2-nitro-5-(prop-1-en-2-yl)pyridine (2.80 g, 17.0 mmol, 69% yield) as a yellow solid. ¹H NMR (400 MHz, DMSO-d₆) δ=8.82 (dd, J=0.8, 2.0 Hz, 1H), 8.33-8.26 (m, 2H), 5.79 (s, 1H), 5.44 (s, 1H), 2.19 (s, 3H). MS (ESI) m/z 165.1 [M+H]⁺

To a mixture of trimethyl sulfoxonium iodide (5.43 g, 24.6 mmol, 1.50 eq) in dimethylsulfoxide (90.0 mL) was added potassium tert-butoxide (2.77 g, 24.6 mmol, 1.50 eq). The reaction mixture was stirred at 20° C. for 1 h. Then 2-nitro-5-(prop-1-en-2-yl)pyridine (2.70 g, 16.4 mmol, 1.00 eq) in tetrahydrofuran (30.0 mL) was added and the reaction mixture was stirred at 20° C. for another 3 h. The reaction mixture was poured into water (500 mL) and extracted with ethyl acetate (3×200 mL). The combined organic phase was separated, washed with brine (100 mL), dried over anhydrous sodium sulfate, filtered and concentrated to give a residue, which was purified by silica gel chromatography (petroleum ether/ethyl acetate=1/0 to 10/1) to afford 5-(1-methylcyclopropyl)-2-nitropyridine (450 mg, 2.53 mmol, 15% yield) as an off-white solid. ¹H NMR (400 MHz, DMSO-d₆) δ=8.51 (d, J=2.4 Hz, 1H), 8.21 (d, J=8.8 Hz, 1H), 7.98 (dd, J=2.4, 8.8 Hz, 1H), 1.48 (s, 3H), 1.12-1.07 (m, 2H), 1.01-0.95 (m, 2H).

To a mixture of 5-(1-methylcyclopropyl)-2-nitropyridine (450 mg, 2.53 mmol, 1.00 eq) in ethyl acetate (10.0 mL) was added palladium on carbon (45.0 mg, 10% purity). The reaction mixture was stirred at 20° C. for 1 h under hydrogen atmosphere. The reaction mixture was filtered and concentrated to give 5-(1-methylcyclopropyl)pyridin-2-amine (380 mg, crude) as a white solid. MS (ESI) m/z 149.2 [M+H]⁺

To a solution of 5-(1-methylcyclopropyl)pyridin-2-amine (360 mg, 2.43 mmol, 1.00 eq) and pyridine (384 mg, 4.86 mmol, 392 uL, 2.00 eq) in acetonitrile (5.00 mL) was added phenyl carbonochloridate (418 mg, 2.67 mmol, 334 uL, 1.10 eq). The reaction mixture was stirred at 20° C. for 1 h. The reaction mixture was concentrated to give a residue. The residue was purified by silica gel chromatography (petroleum ether/ethyl acetate=20/1 to 10/1) to give phenyl (5-(1-methylcyclopropyl)pyridin-2-yl)carbamate (280 mg, 1.04 mmol, 42% yield) as a white solid. ¹H NMR (400 MHz, DMSO-d₆) δ=10.63 (s, 1H), 8.22 (d, J=2.0 Hz, 1H), 7.74-7.70 (m, 1H), 7.66-7.61 (m, 1H), 7.47-7.41 (m, 2H), 7.31-7.25 (m, 1H), 7.21 (d, J=7.6 Hz, 2H), 1.39 (s, 3H), 0.89-0.84 (m, 2H), 0.79-0.74 (m, 2H). MS (ESI) m/z 269.0 [M+H]⁺

A mixture of 3-(6-(hydroxymethyl)-1-oxoisoindolin-2-yl)piperidine-2,6-dione 1 (80.0 mg, 291 umol, 1.00 eq) in dimethyl formamide (1.00 mL) was added sodium hydride (17.5 mg, 437 umol, 60% purity, 1.50 eq) and phenyl (5-(1-methylcyclopropyl)pyridin-2-yl)carbamate (86.0 mg, 41.0 umol, 1.10 eq) at 0° C. The reaction mixture was stirred at 20° C. for 1 h. The reaction mixture was added formic acid (2.00 mL) and filtered to give a filtrate, which was purified by Prep-HPLC (column: 3_Phenomenex Luna C18 75*30 mm*3 um; mobile phase: [water (0.05% hydrochloric acid)-acetonitrile]; B %: 17%-37%, 6 min) and filtered to give a filter cake. The filter cake was lyophilized to give (2-(2,6-dioxopiperidin-3-yl)-3-oxoisoindolin-5-yl)methyl(5-(1-methylcyclopropyl)pyridin-2-yl)carbamate 388 (42.16 mg, 93.0 umol, 31% yield, 99% purity) as a white solid. ¹H NMR (400 MHz, DMSO-d₆) δ=11.00 (s, 1H), 10.26 (s, 1H), 8.16 (d, J=2.0 Hz, 1H), 7.81 (s, 1H), 7.74 (d, J=8.8 Hz, 1H), 7.70-7.66 (m, 1H), 7.65-7.62 (m, 1H), 7.60 (dd, J=2.4, 8.8 Hz, 1H), 5.29 (s, 2H), 5.13 (dd, J=5.2, 13.2 Hz, 1H), 4.51-4.43 (m, 1H), 4.38-4.31 (m, 1H), 3.03-2.84 (m, 1H), 2.60 (br dd, J=2.0, 16.8 Hz, 1H), 2.44-2.35 (m, 1H), 2.06-1.98 (m, 1H), 1.37 (s, 3H), 0.88-0.80 (m, 2H), 0.77-0.70 (m, 21H). MS (ESI) m/z 449.0 [M+H]⁺

Compound 389: 5-chloro-2,3-dihydrobenzofuran (500 mg, 3.23 mmol, 1.00 eq) was added to nitric acid (5.00 mL). The mixture was stirred at −10° C. for 2 h. The reaction mixture was poured into ice water (30.0 mL) slowly. The reaction solution was extracted with ethyl acetate (3×15.0 mL). The organic phases were combined, dried with anhydrous sodium sulfate, and concentrated under reduced pressure to afford 5-chloro-7-nitro-2,3-dihydrobenzofuran (600 mg, 3.01 mmol, 93 yield) as a yellow solid. ¹H NMR (400 MHz, DMSO-d₆) δ=7.92-7.82 (m, 1H), 7.74-7.67 (m, 1H), 4.81 (t, J=8.8 Hz, 2H), 3.31 (t, J=8.8 Hz, 2H).

To a solution of 5-chloro-7-nitro-2,3-dihydrobenzofuran (600 mg, 3.01 mmol, 1.00 eq) in methanol (8.00 mL) was added saturated ammonium chloride (804 mg, 15.0 mmol, 5.00 eq) and iron powder (839 mg, 15.0 mmol, 5.00 eq) and water (8.00 mL). The mixture was stirred at 80° C. for 2 h. The reaction mixture was filtered and the filtrate was concentrated under reduced pressure to afford a residue. The residue was diluted with saturated sodium bicarbonate (60.0 mL) and extracted with ethyl acetate (3×30.0 mL). The combined organic layers were dried over anhydrous sodium sulfate, filtered and concentrated under reduced pressure to afford 5-chloro-2,3-dihydrobenzofuran-7-amine (350 mg, 1.94 mmol, 65% yield) as a brown solid. ¹H NMR (400 MHz, DMSO-d₆) δ=6.45 (s, 2H), 4.92 (s, 2H), 4.48 (t, J=8.8 Hz, 2H), 3.10 (t, J=8.8 Hz, 2H).

To a solution of 5-chloro-2,3-dihydrobenzofuran-7-amine (200 mg, 1.18 mmol, 1.00 eq) in acetonitrile (3.00 mL) was added pyridine (280 mg, 3.54 mmol, 286 uL, 3.00 eq) and phenyl carbonochloridate (185 mg, 1.18 mmol, 148 uL, 1.00 eq). The mixture was stirred at 25° C. for 12 h. The reaction mixture was concentrated under reduced pressure to afford a residue. The residue was purified by reverse-phase HPLC (column: spherical C18, 20-45 um, 100 Å, SW 120, mobile phase:[water (0.1% formic acid)-acetonitrile]) and the desired eluent was lyophilized to afford phenyl (5-chloro-2,3-dihydrobenzofuran-7-yl)carbamate (280 mg, 966 umol, 81% yield) as a white solid. ¹H NMR (400 MHz, DMSO-d₆) δ=9.34 (br s, 1H), 7.19-7.12 (m, 2H), 6.79-6.72 (m, 3H), 6.45 (s, 2H), 4.48 (t, J=8.8 Hz, 2H), 3.10 (t, J=8.8 Hz, 2H).

To a solution of phenyl (5-chloro-2,3-dihydrobenzofuran-7-yl)carbamate (93.0 mg, 321 umol, 1.10 eq) in dimethyl formamide (500 uL) was added sodium hydride (23.3 mg, 583 umol, 60% purity, 2.00 eq) and 3-(6-(hydroxymethyl)-1-oxoisoindolin-2-yl)piperidine-2,6-dione 1 (80.0 mg, 292 umol, 1.00 eq). The mixture was stirred at 25° C. for 1 h. The pH of the mixture was adjusted to 7 with formic acid, then the mixture was diluted with dimethyl formamide (1.00 mL). The residue was purified by Prep-HPLC (column: Phenomenex luna C18 150*25 mm*10 um; mobile phase: [water (0.225% formic acid)-acetonitrile]; B %: 30%-60%, 10 min) and the desired eluent was lyophilized to afford (2-(2,6-dioxopiperidin-3-yl)-3-oxoisoindolin-5-yl)methyl(5-chloro-2,3-dihydrobenzofuran-7-yl)carbamate 389 (68.81 mg, 144 umol, 49% yield, 99% purity) as a white solid. ¹H NMR (400 MHz, DMSO-d₆) δ=10.99 (br s, 1H), 9.33 (br s, 1H), 7.81 (s, 1H), 7.70-7.59 (m, 2H), 7.43 (br s, 1H), 7.04 (d, J=2.0 Hz, 1H), 5.25 (s, 2H), 5.13 (dd, J=5.6, 13.2 Hz, 1H), 4.58 (t, J=8.8 Hz, 2H), 4.51-4.41 (m, 1H), 4.39-4.28 (m, 1H), 3.21 (br t, J=8.8 Hz, 2H), 2.97-2.85 (m, 1H), 2.63-2.57 (m, 1H), 2.40 (br dd, J=4.8, 13.0 Hz, 1H), 2.06-1.95 (m, 1H). MS (ESI) m/z 470.2 [M+H]⁺

Compound 390: A mixture of 3-(6-(hydroxymethyl)-1-oxoisoindolin-2-yl)piperidine-2,6-dione I (80.0 mg, 291 umol, 1.00 eq) in dimethyl formamide (1.00 mL) was added sodium hydride (17.5 mg, 437 umol, 60% purity, 1.50 eq) and phenyl (3-fluorobicyclo[1.1.1]pentan-1-yl)carbamate (67.7 mg, 306 umol, 1.05 eq) at 0° C. The reaction mixture was stirred at 20° C. for 1 h. The reaction mixture was added formic acid 2.00 mL) and filtered to give a filtrate, which was purified by Prep-HPLC (column: Phenomenex luna C18 150*25 mm*10 um; mobile phase: [water (0.225% formic acid)-acetonitrile]; B %: 21%-51%, 10 min) and lyophilized to give (2-(2,6-dioxopiperidin-3-yl)-3-oxoisoindolin-5-yl)methyl(3-fluorobicyclo[1.1.1]pentan-1-yl)carbamate 390 (48.8 mg, 120 umol, 41% yield, 99% purity) as a white solid. ¹H NMR (400 MHz, DMSO-d₆) δ=11.00 (br s, 1H), 8.20 (br s, 1H), 7.71 (s, 1H), 7.61 (s, 2H), 5.14 (br s, 2H), 5.11 (br d, J=5.2 Hz, 1H), 4.51-4.42 (m, 1H), 4.39-4.29 (m, 1H), 2.97-2.86 (m, 1H), 2.61 (br dd, J=2.0, 15.6 Hz, 1H), 2.41 (dq, J=4.8, 13.2 Hz, 1H), 2.27 (s, 6H), 2.07-1.97 (m, 1H). MS (ESI) m/z 401.9 [M+H]⁺

Compound 391: To a solution of 2,6-difluorophenol (0.500 g, 3.84 mmol, 1.00 eq) in dimethyl formamide (5.00 mL) was added sodium 2-chloro-2,2-difluoroacetate (1.17 g, 7.69 mmol, 2.00 eq) and potassium carbonate (640 mg, 4.63 mmol, 1.20 eq). The mixture was stirred at 100° C. for 3 h. The reaction mixture was diluted with water (100 mL) and exacted with ethyl acetate (3×100 mL). The organic phase was separated, washed with brine (2×50.0 mL), dried over sodium sulfate, filtered and concentrated under reduced pressure to give 2-(difluoromethoxy)-1,3-difluorobenzene (500 mg, 2.78 mmol, 72% yield) as a yellow solid. ¹H NMR (400 MHz, CDCl₃) δ=7.24-7.14 (m, 1H), 7.03-6.95 (m, 2H), 6.59 (t, J=73.6 Hz, 1H).

To a solution of sulfuric acid (2.00 mL) was added nitric acid (2.80 g, 28.8 mmol, 2.00 mL, 65% purity, 10.4 eq) at −10° C. Then 2-(difluoromethoxy)-1,3-difluorobenzene (500 mg, 2.78 mmol, 1.00 eq) was added slowly at −10° C. The mixture was stirred at −10° C. for 0.5 h. The reaction mixture was diluted with cold water (100 mL) and extracted with ethyl acetate (3×50.0 mL). The combined organic layer was washed with saturated sodium bicarbonate (50.0 mL), dried over anhydrous sodium sulfate and concentrated under vacuum. The residue was purified by column chromatography (SiO₂, Petroleum ether/Ethyl acetate=20/1 to 0/1) to give 2-(difluoromethoxy)-1,3-difluoro-4-nitrobenzene (170 mg, 755 umol, 27% yield) as yellow oil. ¹H NMR (400 MHz, CDCl₃) δ=8.09 (ddd, J=5.2, 8.0, 9.6 Hz, 1H), 7.17 (ddd, J=2.0, 8.4, 9.6 Hz, 1H), 6.67 (t, J=72.0 Hz, 1H).

To a solution of 2-(difluoromethoxy)-1,3-difluoro-4-nitrobenzene (170 mg, 755 umol, 1.00 eq) in methanol (10.0 mL) and water (2.00 mL) was added iron powder (210 mg, 3.78 mmol, 5.00 eq) and ammonium chloride (323 mg, 6.04 mmol, 8.00 eq). The mixture was stirred at 80° C. for 2 h. The mixture was filtered and the filtrate was concentrated under reduced pressure to give a residue. The crude product was diluted with saturated sodium bicarbonate (30.0 mL) and exacted with ethyl acetate (3×30.0 mL). The organic phase was separated, washed with brine (2×10.0 mL), dried over sodium sulfate, filtered and concentrated under reduced pressure to give 3-(difluoromethoxy)-2,4-difluoroaniline (60.0 mg, 307 umol, 40% yield) as yellow oil.

To a solution of 3-(difluoromethoxy)-2,4-difluoroaniline (60.0 mg, 307 umol, 1.00 eq) in acetonitrile (5.00 mL) was added pyridine (121 mg, 1.54 mmol, 124 uL, 5.00 eq) and phenyl carbonochloridate (52.9 mg, 338 umol, 42.3 uL, 1.10 eq). The mixture was stirred at 25° C. for 1 h. The reaction mixture was concentrated under reduced pressure to give oil. The oil was diluted with water (30.0 mL) and exacted with ethyl acetate (30.0 mL). The organic phase was separated and concentrated under reduced pressure to give a residue. The crude product was purified by prep-TLC (SiO₂, petroleum ether/ethyl acetate=5/1) to give phenyl (3-(difluoromethoxy)-2,4-difluorophenyl)carbamate (90.0 mg, 285 umol, 92% yield) as yellow oil. ¹H NMR (400 MHz, CDCl₃) δ=8.01 (br s, 1H), 7.45-7.39 (m, 2H), 7.26-7.19 (m, 3H), 7.02 (dt, J=2.4, 9.6 Hz, 1H), 6.94 (t, J=7.6 Hz, 1H), 6.62 (t, J=73.2 Hz, 1H). MS (ESI) m/z 316.0 [M+H]⁺

To a solution of phenyl phenyl (3-(difluoromethoxy)-2,4-difluorophenyl)carbamate (90.0 mg, 285 umol, 1.00 eq) in dimethyl formamide (2.00 mL) was added 3-(6-(hydroxymethyl)-1-oxoisoindolin-2-yl) piperidine-2,6-dione (86.1 mg, 314 umol, 1.10 eq) and sodium hydride (22.8 mg, 571 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC (column: Phenomenex Synergi C18 150*25 mm*10 um; mobile phase: [water (0.225% FA)-ACN]; B %: 29%-59%, 10 min) and lyophilized to give (2-(2,6-dioxopiperidin-3-yl)-3-oxoisoindolin-5-yl)methyl (3-(difluoromethoxy)-2,4-difluorophenyl) carbamate 391 (84.14 mg, 169 umol, 59% yield) as a white solid. ¹H NMR (400 MHz, DMSO-d₆) δ=10.99 (br s, 1H), 9.74 (br s, 1H), 7.80 (s, 1H), 7.71-7.55 (m, 3H), 7.30-7.25 (m, 1H), 7.24 (t, J=72.0 Hz, 1H), 5.28 (s, 2H), 5.13 (dd, J=5.2, 13.2 Hz, 1H), 4.51-4.44 (m, 1H), 4.39-4.31 (m, 1H), 2.97-2.86 (m, 1H), 2.65-2.57 (m, 1H), 2.40 (dq, J=4.4, 13.2 Hz, 1H), 2.06-1.97 (m, 1H). MS (ESI) m/z 496.1 [M+H]⁺

Compound 392: To a solution of 5-fluoro-2-nitropyridine (300 mg, 2.11 mmol, 1.00 eq) in dimethyl formamide (2.00 mL) was added (R)-2-methylpyrrolidine (260 mg, 2.14 mmol, 1.01 eq, hydrochloric acid) and potassium carbonate (875 mg, 6.33 mmol, 3.00 eq). The mixture was stirred at 60° C. for 2 h. The reaction mixture was concentrated under reduced pressure to give a residue. The crude product was purified by column chromatography (SiO₂, Petroleum ether/Ethyl acetate=5/1 to 3/1) to give (R)-5-(2-methylpyrrolidin-1-yl)-2-nitropyridine (400 mg, 1.93 mmol, 91% yield) as a yellow solid. ¹H NMR (400 MHz, CDCl₃) δ=8.17 (d, J=9.2 Hz, 1H), 7.86 (d, J=2.8 Hz, 1H), 6.87 (dd, J=2.8, 9.2 Hz, 1H), 4.14-4.01 (m, 1H), 3.61-3.53 (m, 1H), 3.39-3.29 (m, 1H), 2.21-2.10 (m, 3H), 1.91-1.80 (m, 1H), 1.25 (d, J=6.4 Hz, 3H).

To a solution of (R)-5-(2-methylpyrrolidin-1-yl)-2-nitropyridine (400 mg, 1.93 mmol, 1.00 eq) in methanol (20.0 mL) and water (2.00 mL) was added iron power (538 mg, 9.65 mmol, 5.00 eq) and ammonium chloride (826 mg, 15.4 mmol, 8.00 eq). The mixture was stirred at 80° C. for 2 h. The mixture was filtered and the filtrate was concentrated under reduced pressure to give a residue. The crude product was diluted with saturated sodium bicarbonate (30.0 mL) and exacted with ethyl acetate (3×30.0 mL). The organic phase was separated, washed with brine (2×10.0 mL), dried over sodium sulfate, filtered and concentrated under reduced pressure to give (R)-5-(2-methylpyrrolidin-1-yl)pyridin-2-amine (300 mg, 1.69 mmol, 87% yield) as a yellow solid. ¹H NMR (400 MHz, CDCl₃) δ=7.52 (d, J=2.8 Hz, 1H), 6.88 (dd, J=2.8, 8.8 Hz, 1H), 6.50 (d, J=8.8 Hz, 1H), 3.74 (dd, J=2.4, 6.4 Hz, 1H), 3.42-3.36 (m, 1H), 3.08 (q, J=8.4 Hz, 1H), 2.09-1.97 (m, 3H), 1.73-1.65 (m, 1H), 1.14 (d, J=6.4 Hz, 3H). MS (ESI) m/z 178.2 [M+H]⁺

To a solution of (R)-5-(2-methylpyrrolidin-1-yl)pyridin-2-amine (300 mg, 1.69 mmol, 1.00 eq) in acetonitrile (10.0 mL) was added pyridine (669 mg, 8.46 mmol, 683 uL, 5.00 eq) and phenyl carbonochloridate (318 mg, 2.03 mmol, 254 uL, 1.20 eq). The mixture was stirred at 25° C. for 0.5 h. The reaction mixture was concentrated under reduced pressure to give a residue. The crude product was triturated with water (5.00 ml) and dried to give (R)-phenyl (5-(2-methylpyrrolidin-1-yl)pyridin-2-yl)carbamate (300 mg, 1.01 mmol, 59% yield) as a white solid. ¹H NMR (400 MHz, DMSO-d6) δ=10.18 (br s, 1H), 7.66 (d, J=2.8 Hz, 1H), 7.56 (d, J=8.8 Hz, 1H), 7.45-7.36 (m, 2H), 7.27-7.21 (m, 1H), 7.21-7.15 (m, 2H), 7.02 (dd, J=3.2, 9.2 Hz, 1H), 3.92-3.82 (m, 1H), 3.40-3.37 (m, 1H), 3.14-3.04 (m, 1H), 2.07-1.92 (m, 3H), 1.71-1.63 (m, 1H), 1.09 (d, J=6.0 Hz, 3H). MS (ESI) m/z 298.2 [M+H]⁺

To a solution of 3-(6-(hydroxymethyl)-1-oxoisoindolin-2-yl)piperidine-2,6-dione 1 (70.0 mg, 255 umol, 1.00 eq) in dimethyl formamide (2.00 mL) was added (R)-phenyl (5-(2-methylpyrrolidin-1-yl)pyridin-2-yl) carbamate (76.0 mg, 255 umol, 1.00 eq) and sodium hydride (20.4 mg, 510 umol, 60% purity, 2.00 eq) at 0° C. The mixture was stirred at 0° C. for 0.5 h. The reaction mixture was quenched with formic acid (1.00 ml) to give a solution. The solution was purified by prep-HPLC (column: Phenomenex Synergi C18 150*25 mm*10 um; mobile phase: [water (0.225% FA)-ACN]; B %: 13%-46%, 11 min) and lyophilized to give (2-(2,6-dioxopiperidin-3-yl)-3-oxoisoindolin-5-yl)methyl(5-((R)-2-methylpyrrolidin-1-yl)pyridin-2-yl)carbamate 392 (23.8 mg, 45.5 umol, 17% yield, formate) as an off-white solid. ¹H NMR (400 MHz, DMSO-d₆) δ=10.99 (br s, 1H), 9.81 (s, 1H), 7.80 (s, 1H), 7.69-7.57 (m, 4H), 7.00 (dd, J=2.8, 9.2 Hz, 1H), 5.26 (s, 2H), 5.13 (dd, J=5.2, 13.2 Hz, 1H), 4.51-4.43 (m, 1H), 4.38-4.30 (m, 1H), 3.90-3.82 (m, 1H), 3.39-3.37 (m, 1H), 3.08 (q, J=8.4 Hz, 1H), 2.97-2.88 (m, 1H), 2.61 (br dd, J=2.0, 15.2 Hz, 1H), 2.41 (br dd, J=4.4, 13.2 Hz, 1H), 2.06-1.98 (m, 3H), 1.97-1.91 (m, 1H), 1.69-1.60 (m, 1H), 1.09 (d, J=6.0 Hz, 3H). MS (ESI) m/z 478.1 [M+H]⁺

Compound 393: A mixture of phenyl bicyclo[1.1.1]pentan-1-ylcarbamate 1 (20.0 mg, 98.4 umol, 1.00 eq) and 3-(6-(hydroxymethyl)-1-oxoisoindolin-2-yl)piperidine-2,6-dione I (32.4 mg, 118 umol, 1.20 eq) in dimethyl formamide (1.00 mL) was added sodium hydride (5.90 mg, 148 umol, 60% purity, 1.50 eq) at 0° C. The reaction mixture was stirred at 25° C. for 1 h. The reaction mixture was added formic acid (1.00 mL) and filtered to give a filtrate, which was purified by Prep-HPLC (column: Phenomenex Luna C18 150*25 mm*10 um; mobile phase: [water (0.225% formic acid)-acetonitrile]; B %: 19%-49%, 10 min) and lyophilized to give (2-(2,6-dioxopiperidin-3-yl)-3-oxoisoindolin-5-yl)methyl bicyclo[1.1.1]pentan-1-ylcarbamate 393 (19 mg, 49.6 umol, 50% yield, 99% purity) as a white solid. ¹H NMR (400 MHz, DMSO-d₆) δ=10.99 (br s, 1H), 8.02 (br s, 1H), 7.70 (s, 1H), 7.62 (s, 2H), 5.21-5.07 (m, 3H), 4.51-4.42 (m, 1H), 4.40-4.29 (m, 1H), 2.99-2.85 (m, 1H), 2.61 (td, J=2.2, 15.4 Hz, 1H), 2.47-2.33 (m, 2H), 2.06-1.98 (m, 1H), 1.96-1.87 (m, 6H). MS (ESI) m/z 384.1 [M+H]⁺

Compound 394: To a solution of 5-chloro-4-methylpyridin-2-amine (3.00 g, 21.0 mmol, 1.00 eq) in sulfuric acid (30.0 mL) was added hydrogen peroxide (23.8 g, 210 mmol, 20.2 mL, 30% purity, 10.0 eq) dropwise at 0° C. The mixture was stirred at 15° C. for 12 h. The mixture was poured into water (100 mL), then the mixture was quenched with saturated sodium sulfite to potassium iodide starch paper from blue to colorless. Then the mixture was diluted with saturated sodium carbonate (200 mL) and exacted with ethyl acetate (3×100 mL). The organic phase was separated, washed with brine (2×10.0 mL), dried over sodium sulfate, filtered and concentrated under reduced pressure to give a residue. The residue was purified by column chromatography (SiO₂, petroleum ether/ethyl acetate=20/1 to 1/1) to give 5-chloro-4-methyl-2-nitropyridine (2.70 g, 15.6 mmol, 74% yield) as a white solid. ¹H NMR (400 MHz, CDCl₃) δ=8.51 (s, 1H), 8.16 (s, 1H), 2.56 (s, 3H).

To a solution of 5-chloro-4-methyl-2-nitropyridine (1.50 g, 8.69 mmol, 1.00 eq) and pyrrolidine (3.09 g, 43.4 mmol, 3.63 mL, 5.00 eq) in dimethylformamide (15.0 mL) was added potassium carbonate (3.60 g, 26.0 mmol, 3.00 eq). The mixture was stirred at 60° C. for 12 h. The mixture was filtered and the filtrate was concentrated to give crude product. The crude product was purified by silica gel chromatography (petroleum ether/ethyl acetate=10/1 to 1/1) to give 4-methyl-2-nitro-5-(pyrrolidin-1-yl)pyridine (700 mg, 3.38 mmol, 38% yield) as a yellow solid. ¹H NMR (400 MHz, DMSO-d₆) δ=8.01 (s, 1H), 7.82 (s, 1H), 3.65-3.52 (m, 4H), 2.51 (s, 3H), 1.98-1.85 (m, 4H).

To a mixture of 4-methyl-2-nitro-5-(pyrrolidin-1-yl)pyridine (300 mg, 1.45 mmol, 1.00 eq) in methanol (10.0 mL) was added Pd/C (100 mg, 10% purity). The mixture was stirred at 15° C. for 1 h under hydrogen (15 Psi). The mixture was filtered to give a filtrate. The filtrate was concentrated to give 4-methyl-5-(pyrrolidin-1-yl)pyridin-2-amine (340 mg, crude) as yellow oil. ¹H NMR (400 MHz, DMSO-d₆) δ=7.56 (s, 1H), 6.26 (s, 1H), 5.30 (s, 2H), 2.93 (brt, J=6.1 Hz, 4H), 2.11 (s, 3H), 1.82 (td, J=3.1, 6.4 Hz, 4H).

To a mixture of 4-methyl-5-(pyrrolidin-1-yl)pyridin-2-amine (340 mg, 1.92 mmol, 1.00 eq) and pyridine (759 mg, 9.59 mmol, 774 uL, 5.00 eq) in acetonitrile (5.00 mL) was added phenyl carbonochloridate (360 mg, 2.30 mmol, 288 uL, 1.20 eq) dropwise at 0° C. The mixture was stirred at 15° C. for 12 h. The mixture was concentrated in vacuum. The residue was triturated with ethyl acetate (4.00 mL) and filtered to give phenyl (4-methyl-5-(pyrrolidin-1-yl)pyridin-2-yl)carbamate (400 mg, 1.35 mmol, 70% yield) as a yellow solid.

To a mixture of phenyl (4-methyl-5-(pyrrolidin-1-yl)pyridin-2-yl)carbamate (130 mg, 438 umol, 1.20 eq) and 3-(6-(hydroxymethyl)-1-oxoisoindolin-2-yl)piperidine-2,6-dione I (100 mg, 365 umol, 1.00 eq) in dimethyformamide (2.00 mL) was added sodium hydride (21.9 mg, 547 umol, 60% purity, 1.50 eq) in portions. The mixture was stirred at 15° C. for 2 h. The mixture was quenched with 1 M hydrochloric acid (0.500 mL) and filtered. The residue was purified by prep-HPLC (column: 3_Phenomenex Luna C18 75*30 mm*3 um; mobile phase: [water (0.05% HCl)-ACN]; B %: 14%-34%, 9 min) and lyophilized to give (2-(2,6-dioxopiperidin-3-yl)-3-oxoisoindolin-5-yl)methyl(4-methyl-5-(pyrrolidin-1-yl)pyridin-2-yl) carbamate 394 (25.28 mg, 52.4 umol, 14% yield, 99% purity) as a yellow solid. ¹H NMR (400 MHz, DMSO-d₆) δ=11.29 (br d, J=2.9 Hz, 1H), 10.99 (s, 1H), 8.02-7.87 (m, 1H), 7.82 (s, 1H), 7.72-7.69 (m, 1H), 7.67-7.62 (m, 1H), 7.53 (br s, 1H), 5.37 (s, 2H), 5.12 (br dd, J=5.1, 13.3 Hz, 1H), 4.52-4.43 (m, 1H), 4.39-4.31 (m, 1H), 3.39 (br s, 4H), 2.99-2.82 (m, 1H), 2.60 (br d, J=17.4 Hz, 1H), 2.52 (s, 3H), 2.46-2.36 (m, 1H), 2.05-1.95 (m, 5H). MS (ESI) m/z 478.2 [M+H]⁺

Compound 395: To a solution of methyl 4-aminobenzoate (1.00 g, 6.62 mmol, 1.00 eq) in tetrahydrofuran (100 mL) was added methylmagnesium bromide (3.00 M, 11.0 mL, 5.00 eq) (3.00 M in diethyl ether) under nitrogen atmosphere at −40° C. The mixture was stirred at 25° C. for 12 h. Methylmagnesium bromide (3.00 M, 11.0 mL, 5.00 eq) was added into the reaction mixture at −40° C. The mixture was stirred at 25° C. for another 12 h. The reaction mixture was quenched with saturated ammonium chloride (400 mL) at 0° C. and extracted with ethyl acetate (300 mL). The combined organic layer was washed with water and dried over anhydrous sodium sulfate. After filtration, the filtrate was concentrated under reduced pressure to give a residue. The crude product was purified by column chromatography on silica gel eluted with petroleum ether/ethyl acetate=1/0 to 0/1 to give 2-(4-aminophenyl)propan-2-ol (250 mg, 1.65 mmol, 25% yield) as yellow oil. ¹H NMR (400 MHz, DMSO-d₆) δ=7.12-7.07 (m, 2H), 6.49-6.46 (m, 2H), 4.81 (s, 2H), 4.64 (s, 1H), 1.34 (s, 6H). MS (ESI) m/z 152.2 [M+H]⁺

To a solution of 2-(4-aminophenyl)propan-2-ol (130 mg, 860 umol, 1.00 eq) in acetonitrile (5.00 mL) was added pyridine (204 mg, 2.58 mmol, 208 uL, 3.00 eq) and phenyl carbonochloridate (163 mg, 1.04 mmol, 130 uL, 1.21 eq) in portions. The mixture was stirred at 15° C. for 1 h. The reaction mixture was concentrated under reduced pressure to give a residue. The crude product was purified by column chromatography on silica gel eluted with petroleum ether/ethyl acetate=20/1 to 0/1 to give phenyl (4-(2-hydroxypropan-2-yl)phenyl)carbamate (60.0 mg, 221 umol, 26% yield) as a white solid.

¹H NMR (400 MHz, DMSO-d₆) δ=10.12 (brs, 1H), 7.45-7.38 (m, 6H), 7.28-7.19 (m, 3H), 4.93 (s, 1H), 1.40 (s, 6H). MS (ESI) m/z 254.0 [M+H−18]⁺

To a solution of 3-(6-(hydroxymethyl)-1-oxoisoindolin-2-yl)piperidine-2,6-dione 1 (50.0 mg, 182 umol, 1.00 eq) and phenyl (4-(2-hydroxypropan-2-yl)phenyl)carbamate (59.0 mg, 217 umol, 1.19 eq) in N,N-dimethyl formamide (1.00 mL) was added sodium hydride (60%, dispersion in paraffin liquid) (15.0 mg, 375 umol, 60% purity, 2.06 eq) at 0° C. in portions. The mixture was stirred at 15° C. for 1 h. The reaction mixture was quenched with hydrochloric acid (1 M, 15.0 mL) and extracted with ethyl acetate (30.0 mL). The combined organic layer was washed with water (10.0 mL) and dried over anhydrous sodium sulfate. After filtration, the filtrate was concentrated under reduced pressure to give a residue which was dissolved in N,N-dimethyl formamide (2.00 mL) and purified by prep-HPLC (column: Unisil 3-100 C18 Ultra 150*50 mm*3 um; mobile phase: [water (0.225% FA)-ACN]; B %: 20%-50%, 10 min). The desired fraction was collected and concentrated under pressure to give a solution. The residual solution was lyophilized to give (2-(2,6-dioxopiperidin-3-yl)-3-oxoisoindolin-5-yl)methyl (4-(2-hydroxypropan-2-yl)phenyl)carbamate 395 (45.01 mg, 89.6 umol, 49% yield, 99% purity, formic acid) as a white solid. ¹H NMR (400 MHz, DMSO-d₆) δ=10.99 (br s, 1H), 9.72 (br s, 1H), 7.79 (s, 1H), 7.70-7.59 (m, 2H), 7.44-7.30 (m, 4H), 5.26 (s, 2H), 5.13 (dd, J=5.1, 13.3 Hz, 1H), 4.91 (s, 1H), 4.52-4.42 (m, 1H), 4.39-4.28 (m, 1H), 2.98-2.86 (m, 1H), 2.63-2.56 (m, 1H), 2.40 (dd, J=4.5, 13.1 Hz, 1H), 2.05-1.96 (m, 1H), 1.38 (s, 6H). MS (ESI) m/z 434.2 [M+H−18]⁺

Compound 396: To a solution of tert-butyl (3-aminobicyclo[1.1.1]pentan-1-yl)carbamate (100 mg, 504 umol, 1.00 eq) in acetonitrile (2.00 mL) was added cesium carbonate (493 mg, 1.51 mmol, 3.00 eq) and phenyl carbonochloridate (102 mg, 655 umol, 82.1 uL, 1.30 eq). The mixture was stirred at 25° C. for 1 h. The mixture was filtered to give a filter liquor which was concentrated under reduced pressure to give tert-butyl phenyl bicyclo[1.1.1]pentane-1,3-diyldicarbamate (60.0 mg, 188 umol, 37% yield) as a white solid. ¹H NMR (400 MHz, DMSO-d₆) δ=9.36-9.27 (m, 1H), 7.18-7.14 (m, 2H), 6.79-6.73 (m, 3H), 2.06-1.97 (m, 4H), 1.81 (s, 2H), 1.37 (s, 9H).

To a solution of tert-butyl phenyl bicyclo[1.1.1]pentane-1,3-diyldicarbamate (50.0 mg, 157 umol, 1.00 eq) in dimethyl formamide (2.00 mL) was added 3-(6-(hydroxymethyl)-1-oxoisoindolin-2-yl)piperidine-2,6-dione (43.0 mg, 157 umol, 1.00 eq) and sodium hydride (12.5 mg, 314 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC (column: Phenomenex Synergi C18 150*25 mm*10 um; mobile phase: [water (0.225% FA)-ACN]; B %: 25%-52%, 9 min) and lyophilized to give tert-butyl((2-(2,6-dioxopiperidin-3-yl)-3-oxoisoindolin-5-yl)methyl)bicyclo[1.1.1]pentane-1,3-diyldicarbamate 396 (10.3 mg, 18.9 umol, 12% yield, formate) as a white solid. ¹H NMR (400 MHz, DMSO-d₆) δ=10.99 (br s, 1H), 8.01 (br s, 1H), 7.69 (s, 1H), 7.60 (s, 2H), 7.56-7.43 (m, 1H), 5.20-5.05 (m, 3H), 4.49-4.41 (m, 1H), 4.37-4.29 (m, 1H), 2.96-2.86 (m, 1H), 2.63-2.57 (m, 1H), 2.40 (br dd, J=4.4, 13.2 Hz, 1H), 2.12-1.97 (m, 7H), 1.36 (br s, 9H). MS (ESI) m/z 443.0 [M+H−56]⁺

Compound 397: To a mixture of 5-bromo-2-fluoro-4-methylaniline (300 mg, 1.47 mmol, 1.00 eq) and pyridine (349 mg, 4.41 mmol, 356 uL, 3.00 eq) in acetonitrile (3.00 mL) was added phenyl carbonochloridate (253 mg, 1.62 mmol, 203 uL, 1.10 eq) at 0° C. After addition, the mixture was stirred at 25° C. for 2 h. The reaction mixture was poured into water (30.0 mL) and extracted with ethyl acetate (2×50.0 mL). The combined organic layers were washed with brine (2×25.0 mL), dried over anhydrous sodium sulfate, filtered and concentrated under reduced pressure to give a residue. The crude product was purified by silica gel chromatography (petroleum ether/ethyl acetate=20/1 to 10/1) to afford phenyl (5-bromo-2-fluoro-4-methylphenyl)carbamate (330 mg, 967 umol, 66% yield, 95% purity) as a white solid. ¹H NMR (400 MHz, CDCl₃) δ=8.37 (s, 1H), 7.46-7.41 (m, 2H), 7.32-7.29 (m, 1H), 7.24-7.20 (m, 2H), 7.11 (s, 1H), 7.03 (d, J=11.6 Hz, 1H), 2.37 (s, 3H).

To a solution of 3-(6-(hydroxymethyl)-1-oxoisoindolin-2-yl)piperidine-2,6-dione 1 (100 mg, 365 umol, 1.00 eq) in tetrahydrofuran (2.00 mL) was added sodium hydride (20.0 mg, 500 umol, 60% purity, 1.37 eq) at 0° C. under nitrogen atmosphere. After stirring the mixture at 0° C. for 15 min, phenyl (5-bromo-2-fluoro-4-methylphenyl)carbamate (130 mg, 401 umol, 1.10 eq) was added at 0° C. After addition, the mixture was stirred at 25° C. for 1 h. The reaction mixture was poured into water (30.0 mL) and extracted with ethyl acetate (2×50.0 mL). The combined organic layers were washed with brine (2×25.0 mL), dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 35%-68%, 11 min) to afford (2-(2,6-dioxopiperidin-3-yl)-3-oxoisoindolin-5-yl)methyl (5-bromo-2-fluoro-4-methylphenyl)carbamate 397 (66.07 mg, 131 umol, 36% yield, 99% purity) as a white solid. ¹H NMR (400 MHz, DMSO-d₆) δ=11.00 (s, 1H), 9.67 (s, 1H), 7.91 (d, J=7.6 Hz, 1H), 7.80 (s, 1H), 7.69-7.62 (m, 2H), 7.30 (d, J=11.6 Hz, 2H), 5.27 (s, 2H), 5.10 (dd, J=5.2, 13.6 Hz, 1H), 4.49-4.31 (m, 2H), 2.99-2.85 (m, 1H), 2.65-2.57 (m, 1H), 2.47-2.35 (m, 1H), 2.30 (s, 3H), 2.05-1.97 (m, 1H). MS (ESI) m/z 505.5 [M+H]⁺

Compound 398: A mixture of phenyl (3-(piperidin-1-yl)bicyclo[1.1.1]pentan-1-yl)carbamate (20.0 mg, 69.8 umol, 1.00 eq) and 3-(6-(hydroxymethyl)-1-oxoisoindolin-2-yl)piperidine-2,6-dione 1 (23.0 mg, 83.8 umol, 1.20 eq) in dimethyl formamide (1.00 mL) was added sodium hydride (5.59 mg, 140 umol, 60% purity, 2.00 eq) at 0° C. The reaction mixture was stirred at 25° C. for 1 h. The reaction mixture was added formic acid (1.00 mL) and filtered to give a filtrate, which was purified by Prep-HPLC (column: Phenomenex Luna C18 150*25 mm*10 um; mobile phase: [water (0.225% formic acid)-acetonitrile]; B %: 1%-30%, 10 min) and lyophilized to give (2-(2,6-dioxopiperidin-3-yl)-3-oxoisoindolin-5-yl)methyl(3-(piperidin-1-yl)bicyclo[1.1.1] pentan-1-yl)carbamate 398 (8.54 mg, 18.1 umol, 26% yield, 99% purity) as a white solid. ¹H NMR (400 MHz, DMSO-d₆) δ=11.00 (s, 1H), 8.08-7.95 (m, 1H), 7.70 (s, 1H), 7.61 (s, 2H), 5.17-5.08 (m, 3H), 4.51-4.42 (m, 1H), 4.38-4.30 (m, 1H), 2.98-2.86 (m, 1H), 2.61 (br d, J=17.0 Hz, 1H), 2.48-2.41 (m, 1H), 2.41-2.28 (m, 4H), 2.05-1.98 (m, 1H), 1.88 (br s, 6H), 1.49 (br s, 4H), 1.40-1.32 (m, 2H). MS (ESI) m/z 467.2 [M+H]⁺

Compound 399: To a mixture of methyl 5-aminopicolinate (1.00 g, 6.57 mmol, 1.00 eq) in tetrahydrofuran (10.0 mL) was added methylmagnesium bromide (3 mol/L in ethyl ether, 9.20 mL, 4.20 eq) drop-wise at −30° C. under nitrogen. After stirring the mixture at 10° C. for 1 h, the reaction was quenched with saturated ammonium chloride (50.0 mL) and extracted with ethyl acetate (3×50.0 mL). The combined organic layers were dried over sodium sulfate, filtered and concentrated to give the crude product. The residue was purified by flash silica gel chromatography (ISCO®; 20 g SepaFlash® Silica Flash Column, Eluent of 0-80% ethyl acetate/petroleum ether gradient @ 40 mL/min) to afford 2-(5-aminopyridin-2-yl)propan-2-ol (500 mg, 3.29 mmol, 50% yield) as yellow solid. ¹H NMR (400 MHz, DMSO-d₆) δ=7.82 (d, J=2.8 Hz, 1H), 7.26 (d, J=8.8 Hz, 1H), 6.89 (dd, J=2.8, 8.8 Hz, 1H), 5.07 (s, 2H), 4.90 (s, 1H), 1.35 (s, 6H).

To a mixture of 2-(5-aminopyridin-2-yl)propan-2-ol (300 mg, 1.97 mmol, 1.00 eq), pyridine (311 mg, 3.94 mmol, 2.00 eq) in tetrahydrofuran (10.0 mL) was added phenyl carbonochloridate (326 mg, 2.08 mmol, 1.06 eq) at 0° C. After addition, the mixture was stirred at 0° C. for 1 h. The reaction mixture was diluted with ethyl acetate (30.0 mL) and washed with brine (2×30.0 mL). The separated organic layer was dried over anhydrous sodium sulfate, filtered and concentrated to give the crude product. The crude product was purified by flash silica gel chromatography (ISCO®; 20 g SepaFlash® Silica Flash Column, Eluent of 0-30% ethyl acetate/petroleum ether gradient @ 30 mL/min) to afford phenyl (6-(2-hydroxypropan-2-yl)pyridin-3-yl)carbamate (300 mg, 1.10 mmol, 55% yield) as a white solid. ¹H NMR (400 MHz, CDCl₃) δ=8.45 (d, J=2.0 Hz, 1H), 7.98 (d, J=6.8 Hz, 1H), 7.38-7.29 (m, 3H), 7.21-7.17 (m, 1H), 7.15-7.09 (m, 2H), 1.48 (s, 6H).

To a mixture of 3-(6-(hydroxymethyl)-1-oxoisoindolin-2-yl)piperidine-2,6-dione 1 (100 mg, 364.60 μmol, 1.00 eq) in tetrahydrofuran (5.00 mL) was added sodium hydride (25 mg, 625.06 μmol, 60% purity, 1.71 eq), and then the mixture was stirred at 0° C. for 0.5 h. Phenyl (6-(2-hydroxypropan-2-yl)pyridin-3-yl)carbamate (100 mg, 367.24 μmol, 1.01 eq) was added to the above mixture and then the resulting mixture was stirred at 0° C. for another 1 h. The mixture was poured into saturated ammonium chloride (30.0 mL) and extracted with ethyl acetate (3×30.0 mL). The organic layers were dried over anhydrous sodium sulfate, filtered and concentrated to give the crude product. The crude product was purified by prep-HPLC (column: Unisil 3-100 C18 μLtra 150*50 mm*3 um; mobile phase: [water (0.225% FA)-ACN]; B %: 1%-30%, 10 min). The eluent was concentrated to remove organic solvents, and then the aqueous solution was lyophilized to give (2-(2,6-dioxopiperidin-3-yl)-3-oxoisoindolin-5-yl)methyl (6-(2-hydroxypropan-2-yl)pyridin-3-yl)carbamate 399 (112 mg, 225 μmol, 61% yield, 99% purity) as a white solid. 108.46 mg of the final product has been delivered and the rest of it has been used to run analytic data. ¹H NMR (400 MHz, DMSO-d₆) δ=10.99 (s, 1H), 9.95 (s, 1H), 8.52 (d, J=2.0 Hz, 1H), 7.89-7.82 (m, 1H), 7.80 (s, 1H), 7.71-7.66 (m, 1H), 7.65-7.61 (m, 1H), 7.56 (d, J=8.8 Hz, 1H), 5.29 (s, 2H), 5.12 (dd, J=5.2, 13.6 Hz, 1H), 4.50-4.44 (m, 1H), 4.38-4.31 (m, 1H), 2.96-2.87 (m, 1H), 2.64-2.57 (m, 1H), 2.45-2.35 (m, 1H), 2.05-1.97 (m, 1H), 1.40 (s, 6H). MS (ESI) m/z 453.1 [M+H]⁺

Compound 400: To a mixture of 2,4-difluoro-5-(trifluoromethoxy)aniline (300 mg, 1.41 mmol, 1.00 eq) and pyridine (167 mg, 2.11 mmol, 1.50 eq) in tetrahydrofuran (10.0 mL) was added phenyl carbonochloridate (231 mg, 1.48 mmol, 1.05 eq) dropwise at 0° C. The mixture was stirred at 25° C. for 0.5 h. The reaction mixture was poured into hydrochloric acid (50.0 mL, 0.5 mol/L) and extracted with ethyl acetate (3×20.0 mL). The organic phase was concentrated under vacuum to give a residue. The residue was purified by silica gel chromatography (petroleum ether/ethyl acetate=50/1 to 20/1) to give phenyl (2,4-difluoro-5-(trifluoromethoxy)phenyl)carbamate (400 mg, 1.02 mmol, 72% yield) as a white solid. ¹H NMR (400 MHz, CDCl₃) δ=8.25 (s, 1H), 7.44-7.41 (m, 2H), 7.30 (d, J=7.6 Hz, 1H), 7.21-7.19 (m, 2H), 7.15 (s, 1H), 7.09-7.04 (m, 1H).

A mixture of 3-(6-(hydroxymethyl)-1-oxoisoindolin-2-yl)piperidine-2,6-dione 1 (61.5 mg, 224 umol, 1.10 eq) in tetrahydrofuran (5.00 mL) was purged with nitrogen for 3 times, and then sodium hydride (12.2 mg, 306 umol, 60% purity, 1.50 eq) was added at 0° C. After stirring the mixture at 0° C. for 0.2 h, phenyl (2,4-difluoro-5-(trifluoromethoxy)phenyl)carbamate (80.0 mg, 204 umol, 1.00 eq) was added. After addition, the mixture was stirred at 0° C. for 0.3 h. The reaction mixture was poured into saturated ammonium chloride (50.0 ml) and extracted with ethyl acetate (3×20.0 mL). The organic phase was concentrated under vacuum to give a residue. The residue was purified by prep-HPLC (column: Phenomenex Synergi C18 150*25 mm*10 um; mobile phase: [water (0.225% FA)-ACN]; B %: 36%-69%, 11 min) and lyophilized to give (2-(2,6-dioxopiperidin-3-yl)-3-oxoisoindolin-5-yl)methyl (2,4-difluoro-5-(trifluoromethoxy)phenyl)carbamate 400 (74.3 mg, 143 umol, 70% yield, 99% purity) as a white solid. ¹H NMR (400 MHz, DMSO-d₆) δ=11.00 (s, 1H), 9.89 (s, 1H), 7.97-7.93 (m, 1H), 7.82 (s, 1H), 7.73-7.67 (m, 2H), 7.66-7.60 (m, 1H), 5.30 (s, 2H), 5.15-5.10 (m, 1H), 4.52-4.44 (m, 1H), 4.38-4.30 (m, 1H), 2.99-2.85 (m, 1H), 2.63-2.58 (m, 1H), 2.45-2.35 (m, 1H), 2.06-1.96 (m, 1H). MS (ESI) m/z: 514.0 [M+H]⁺

Compound 401: To a solution of 4-fluoro-2-methylphenol (10.0 g, 79.2 mmol, 1.00 eq) in tetrahydrofuran (100 mL) was added sodium hydride (3.17 g, 79.2 mmol, 60% purity, 1.00 eq) at 0° C., after gas evolution ceased, methyl carbonochloridate (7.49 g, 79.2 mmol, 6.14 mL, 1.00 eq) was added. The reaction was stirred at 25° C. for 3 h. The mixture was washed with cooled water (100 mL), extracted with ethyl acetate (3×100 mL). The combined organic layers were concentrated to afford 4-fluoro-2-methylphenyl methyl carbonate (14.5 g, 78.7 mmol, 99% yield) as brown oil. ¹H NMR (400 MHz, DMSO-d₆) δ=7.27-7.17 (m, 2H), 7.12-7.04 (m, 1H), 3.84 (s, 3H), 2.15 (s, 3H).

To a solution of 4-fluoro-2-methylphenyl methyl carbonate (14.5 g, 78.7 mmol, 1.00 eq) in sulfuric acid (100 mL) was added potassium nitrate (7.96 g, 78.7 mmol, 1.00 eq) at 0° C. under nitrogen atmosphere, the mixture was stirred at 25° C. for 2 h. The mixture was poured onto water (300 mL) and extracted with ethyl acetate (3×100 mL), the combined organic layers were concentrated to afford 4-fluoro-2-methyl-5-nitrophenyl methyl carbonate (18.0 g, 78.5 mmol, 99% yield) as a red solid. ¹H NMR (400 MHz, DMSO-d₆) δ=8.17 (d, J=6.8 Hz, 1H), 7.63 (d, J=12.0 Hz, 1H), 3.87 (s, 3H), 2.27 (s, 3H).

To a solution of 4-fluoro-2-methyl-5-nitrophenyl methyl carbonate (17.9 g, 78.1 mmol, 1.00 eq) in methanol (160 mL) was added lithium hydroxide (3.74 g, 156 mmol, 2.00 eq), the mixture was stirred at 25° C. for 3 h. The reaction was filtered to give a filtrate. The filtrate was concentrated to give a residue. The residue was purified by flash silica gel chromatography (ISCO®; 20 g SepaFlash® Silica Flash Column, Eluent of 0˜5% petroleum ether/ethyl acetate @ 100 mL/min) to afford 4-fluoro-2-methyl-5-nitrophenol (12.0 g, 70.1 mmol, 89% yield) as a red solid. ¹H NMR (400 MHz, DMSO-d₆) δ=10.32 (br s, 1H), 7.45 (d, J=6.6 Hz, 1H), 7.30 (d, J=12.1 Hz, 1H), 2.19 (s, 3H).

To a solution of 4-fluoro-2-methyl-5-nitro-phenol (1.00 g, 5.84 mmol, 1.00 eq) in dimethylformamide (10.5 mL) and water (1.50 mL) were added potassium carbonate (1.62 g, 11.6 mmol, 2.00 eq) and sodium 2-chloro-2,2-difluoroacetate (4.45 g, 29.2 mmol, 5.00 eq), the mixture was stirred at 100° C. for 12 h. The mixture was dissolved in water (50.0 mL) and extracted with ethyl acetate (3×20.0 mL), the combined organic layers were dried over anhydrous sodium sulfate and concentrated to give a residue. The residue was purified by column chromatography (silica gel, Petroleum ether/Ethyl acetate=20/1 to 10/1) to afford 1-(difluoromethoxy)-4-fluoro-2-methyl-5-nitrobenzene (120 mg, 542 umol, 9% yield) as yellow oil.

To a solution of 1-(difluoromethoxy)-4-fluoro-2-methyl-5-nitrobenzene (120 mg, 542 umol, 1.00 eq) in methanol (1.00 mL) and water (1.00 mL) were added ammonium chloride (145 mg, 2.71 mmol, 5.00 eq) and iron powder (151 mg, 2.71 mmol, 5.00 eq), the reaction was stirred at 80° C. for 2 h. The reaction was filtered to give a filtrate, the filtrate was concentrated to give a residue. The residue was poured into water (50.0 mL) and extracted with ethyl acetate (3×20.0 mL), the combined organic layers were concentrated to afford 5-(difluoromethoxy)-2-fluoro-4-methylaniline (90.0 mg, 470 umol, 86% yield) as yellow oil. MS (ESI) m/z 192.0 [M+H]⁺

To a solution of 5-(difluoromethoxy)-2-fluoro-4-methylaniline (90.0 mg, 470 umol, 1.00 eq) in acetonitrile (1.00 mL) were added phenyl carbonochloridate (77.4 mg, 494 umol, 61.9 uL, 1.05 eq) and pyridine (74.5 mg, 941 umol, 76.0 uL, 2.00 eq), the mixture was stirred at 25° C. for 2 h. The reaction was concentrated to give a residue. The residue was purified by reversed-phase HPLC (column: Shim-pack C18 150*25*10 um; mobile phase: [water (0.1% formic acid)-acetonitrile]) to afford phenyl (5-(difluoromethoxy)-2-fluoro-4-methylphenyl)carbamate (100 mg, 321 umol, 68% yield) as yellow oil. MS (ESI) m/z 312.0 [M+H]⁺

To a solution of 3-(6-(hydroxymethyl)-1-oxoisoindolin-2-yl)piperidine-2,6-dione 1 (80.0 mg, 291 umol, 1.00 eq) in dimethylformamide (1.00 mL) were added phenyl (5-(difluoromethoxy)-2-fluoro-4-methylphenyl) carbamate (99.8 mg, 320 umol, 1.10 eq) and sodium hydride (23.3 mg, 583 umol, 60% purity, 2.00 eq), the mixture was stirred at 25° C. for 1 h. The pH of the mixture was adjusted to 7 with formic acid (0.500 mL), then the mixture was diluted with dimethylformamide (1.00 mL). The reaction was purified by Prep-HPLC (column: Phenomenex luna C18 150*25 mm*10 um; mobile phase: [water (0.225% formic acid)-acetonitrile]; B %: 31%-61%, 10 min) to afford (2-(2,6-dioxopiperidin-3-yl)-3-oxoisoindolin-5-yl) methyl (5-(difluoromethoxy)-2-fluoro-4-methylphenyl)carbamate 401 (77.1 mg, 155 umol, 53% yield, 99% purity) as a white solid. ¹H NMR (400 MHz, DMSO-d₆) δ=10.99 (br s, 1H), 9.65 (br s, 1H), 7.80 (s, 1H), 7.69-7.61 (m, 2H), 7.56 (br d, J=6.2 Hz, 1H), 7.22 (d, J=11.4 Hz, 1H), 7.09 (t, J=74 Hz, 1H), 5.28 (s, 2H), 5.12 (dd, J=5.2, 13.2 Hz, 1H), 4.51-4.44 (m, 1H), 4.38-4.30 (m, 1H), 2.97-2.86 (m, 1H), 2.60 (td, J=1.9, 15.4 Hz, 1H), 2.46-2.35 (m, 1H), 2.18 (s, 3H), 2.06-1.98 (m, 1H). MS (ESI) m/z 491.9 [M+H]⁺

Compound 402: To a solution of 3-(fluoromethyl)bicyclo[1.1.1]pentan-1-amine (180 mg, 1.19 mmol, 1.00 eq, hydrochloric acid) and pyridine (470 mg, 5.94 mmol, 479 uL, 5.00 eq) in acetonitrile (5.00 mL) was added phenyl carbonochloridate (558 mg, 3.56 mmol, 446 uL, 3.00 eq) at 0° C. The mixture was stirred at 20° C. for 1 h. The mixture was concentrated under reduced pressure to give a residue. The residue was triturated with dimethylformamide (1 mL) and water (2 mL), filtered and the filter cake was concentrated to afford phenyl (3-(fluoromethyl)bicyclo[1.1.1]pentan-1-yl)carbamate (160 mg, 680 umol, 57% yield) as a brown solid. ¹H NMR (400 MHz, DMSO-d₆) δ=8.50 (br s, 1H), 7.40-7.34 (m, 2H), 7.24-7.17 (m, 1H), 7.09 (br d, J=7.8 Hz, 2H), 4.55 (s, 1H), 4.43 (s, 1H), 1.96 (s, 6H).

To a solution of 3-(6-(hydroxymethyl)-1-oxoisoindolin-2-yl)piperidine-2,6-dione 1 (80.0 mg, 292 umol, 1.00 eq) and phenyl (3-(fluoromethyl)bicyclo[1.1.1]pentan-1-yl)carbamate (82.3 mg, 350 umol, 1.20 eq) in dimethylformamide (1.00 mL) was added sodium hydride (17.5 mg, 438 umol, 60% purity, 1.50 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 um; mobile phase: [water (0.225% formic acid)-acetonitile]; B %: 21%-51%, 10 min) and lyophilized to afford (2-(2,6-dioxopiperidin-3-yl)-3-oxoisoindolin-5-yl)methyl (3-(fluoromethyl)bicyclo[1.1.1]pentan-1-yl) carbamate 402 (69.3 mg, 163 umol, 56% yield, 98% purity) as a white solid. ¹H NMR (400 MHz, DMSO-d₆) δ=10.99 (s, 1H), 8.07 (br s, 1H), 7.70 (s, 1H), 7.60 (s, 2H), 5.20-5.07 (m, 3H), 4.53 (s, 1H), 4.49-4.43 (m, 1H), 4.41 (s, 1H), 4.37-4.28 (m, 1H), 2.97-2.86 (m, 1H), 2.63-2.57 (m, 1H), 2.45-2.31 (m, 1H), 2.03-1.98 (m, 1H), 1.91 (s, 6H). MS (ESI) m/z 416.1 [M+H]⁺

Compound 403: To a solution of 3-(methoxycarbonyl)bicyclo[1.1.1]pentane-1-carboxylic acid (100 mg, 588 umol, 1.00 eq) in dioxane (5.00 mL) was added diphenylphosphoryl azide (243 mg, 881 umol, 191 uL, 1.50 eq) and triethylamine (119 mg, 1.18 mmol, 164 uL, 2.00 eq). The mixture was stirred at 25° C. for 1 h. Then 3-(6-(hydroxymethyl)-1-oxoisoindolin-2-yl)piperidine-2,6-dione 1 (161 mg, 588 umol, 1.00 eq) was added into the mixture. The reaction mixture was stirred at 80° C. for 2 h under nitrogen. The mixture was concentrated to give a residue. The residue was purified by prep-HPLC (column: Phenomenex Synergi C18 150*25 mm*10 um; mobile phase: [water (0.225% FA)-ACN]; B %: 16%-46%, 10 min). The desired fraction was collected and lyophilized to give a residue. The residue was further purified by column chromatography on silica gel (ethyl acetate) and prep-HPLC (column: Phenomenex Synergi C18 150*25 mm*10 um; mobile phase: [water (0.225% FA)-ACN]; B %: 17%-47%, 10 min). The desired fraction was collected and lyophilized to give methyl 3-((((2-(2,6-dioxopiperidin-3-yl)-3-oxoisoindolin-5-yl) methoxy)carbonyl)amino)bicyclo[1.1.1]pentane-1-carboxylate 403 (51.39 mg, 115 umol, 46% yield, 99% purity) as a white solid. ¹H NMR (400 MHz, DMSO-d₆) δ=10.99 (s, 1H), 8.16 (br s, 1H), 7.56-7.73 (m, 3H), 5.07-5.17 (m, 3H), 4.42-4.50 (m, 1H), 4.29-4.37 (m, 1H), 3.60 (s, 3H), 2.86-2.97 (m, 1H), 2.60 (br dd, 1=−15.53, 2.08 Hz, 1H), 2.40 (br dd, J=−13.20, 4.40 Hz, 1H), 2.14-2.22 (m, 6H), 1.97-2.05 (m, 1H). MS (ESI) m/z 442.2 [M+H]⁺

Compound 404: To a solution of 4-fluoro-2-methyl-5-nitrophenol (1.00 g, 5.84 mmol, 1.00 eq) in dimethyl formamide (10.0 mL) was added sodium hydride (467 mg, 11.6 mmol, 60% purity, 2.00 eq) in portions at 0° C. The mixture was stirred at 0° C. for 0.5 h. Then the mixture was added dibromodifluoromethane (3.68 g, 17.5 mmol, 1.62 mL, 3.00 eq) dropwise at 0° C. and stirred at 25° C. for 2 h. The reaction was quenched by addition ammonium chloride (20.0 mL), extracted with ethyl acetate (3×30.0 mL), the combined organic layers were concentrated to give a residue. The residue was purified by flash silica gel chromatography (40 g SepaFlash® Silica Flash Column, Eluent of 0˜50% Ethyl acetate/Petroleum ether gradient @ 100 mL/min) to afford 1-(bromodifluoromethoxy)-4-fluoro-2-methyl-5-nitrobenzene (710 mg, 2.37 mmol, 40% yield) as yellow oil. ¹H NMR (400 MHz, DMSO-d₆) δ=8.07 (d, J=6.5 Hz, 1H), 7.77 (d, J=11.9 Hz, 1H), 2.38 (s, 3H).

To a solution of 1-(bromodifluoromethoxy)-4-fluoro-2-methyl-5-nitrobenzene (710 mg, 2.37 mmol, 1.00 eq) in dichloromethane (7.00 mL) was added silver tetrafluoroborate (691 mg, 3.55 mmol, 1.50 eq), the mixture was stirred at 25° C. for 2 h. The reaction was filtered to give a filtrate. The filtrate was concentrated to afford 1-fluoro-5-methyl-2-nitro-4-(trifluoromethoxy)benzene (500 mg, 2.09 mmol, 88% yield) as brown oil. ¹H NMR (400 MHz, DMSO-d₆) δ=8.13 (d, J=6.5 Hz, 1H), 7.75 (d, J=11.9 Hz, 1H), 2.39 (s, 3H). To a solution of 1-fluoro-5-methyl-2-nitro-4-(trifluoromethoxy)benzene (250 mg, 1.05 mmol, 1.00 eq) in ethyl acetate (10.0 mL) was added palladium/carbon (50.0 mg, 10% purity), the mixture was stirred at 25° C. for 12 h under hydrogen atmosphere. The reaction was filtered to give a filtrate, the filtrate was concentrated to afford 2-fluoro-4-methyl-5-(trifluoromethoxy)aniline (210 mg, 1.00 mmol, 96% yield) as a yellow solid. MS (ESI) m/z 209.9 [M+H]⁺

To a solution of 2-fluoro-4-methyl-5-(trifluoromethoxy)aniline (210 mg, 1.00 mmol, 1.00 eq) in acetonitrile (3.00 mL) were added phenyl carbonochloridate (165 mg, 1.05 mmol, 132 uL, 1.05 eq) and pyridine (158 mg, 2.01 mmol, 162 uL, 2.00 eq), the mixture was stirred at 25° C. for 2 h. The reaction was concentrated to give a residue. The residue was purified by reversed-phase HPLC (column: Shim-pack C18 150*25*10 um; mobile phase: [water (0.1% formic acid)-acetonitrile]) to afford phenyl (2-fluoro-4-methyl-5-(trifluoromethoxy)phenyl)carbamate (200 mg, 607 umol, 60% yield) as yellow oil. MS (ESI) m/z 330.0 [M+H]⁺

To a solution of 3-(6-(hydroxymethyl)-1-oxoisoindolin-2-yl)piperidine-2,6-dione 1 (80.0 mg, 291 umol, 1.00 eq) in dimethylformamide (1.00 mL) were added phenyl (2-fluoro-4-methyl-5-(trifluoromethoxy)phenyl) carbamate (105 mg, 320 umol, 1.10 eq) and sodium hydride (23.3 mg, 583 umol, 60% purity, 2.00 eq), the mixture was stirred at 25° C. for 1 h. The pH of the mixture was adjusted to 7 with formic acid (0.500 mL), then the mixture was diluted with dimethylformamide (1.00 mL). The reaction was purified by Prep-HPLC (column: Phenomenex luna C18 150*25 mm*10 um; mobile phase: [water (0.225% formic acid)-acetonitrile]; B %: 33%-63%, 10 min) to afford (2-(2,6-dioxopiperidin-3-yl)-3-oxoisoindolin-5-yl) methyl (2-fluoro-4-methyl-5-(trifluoromethoxy)phenyl)carbamate 404 (60.6 mg, 117 umol, 40% yield, 99% purity) as a white solid. ¹H NMR (400 MHz, DMSO-d₆) δ=11.01 (s, 1H), 9.80 (br s, 1H), 7.81 (s, 1H), 7.76 (br d, J=5.4 Hz, 1H), 7.70-7.60 (m, 2H), 7.33 (d, J=11.4 Hz, 1H), 5.29 (s, 2H), 5.13 (dd, J=5.1, 13.3 Hz, 1H), 4.53-4.43 (m, 1H), 4.38-4.30 (m, 1H), 2.98-2.87 (m, 1H), 2.60 (br d, J=17.7 Hz, 1H), 2.46-2.35 (m, 1H), 2.22 (s, 3H), 2.05-1.97 (m, 1H). MS (ESI) m/z 509.9 [M+H]⁺

Compound 405: To a solution of 5-bromo-2-fluoroaniline (500 mg, 2.63 mmol, 1.00 eq), potassium trifluoro(prop-1-en-2-yl) borate (1.17 g, 7.89 mmol, 3.00 eq) and tetrakis[triphenylphosphine]palladium(0) (304 mg, 263 umol, 0.100 eq) in dioxane (10.0 mL) was added cesium carbonate (2.57 g, 7.89 mmol, 3.00 eq), then evacuated with vacuum and back filled with nitrogen 3 times. The mixture was stirred at 100° C. for 12 h. The reaction mixture was filtered and concentrated to give a residue, which was purified by silica gel chromatography (petroleum ether/ethyl acetate=20/1 to 10/1) to afford 2-fluoro-5-(prop-1-en-2-yl)aniline (270 mg, 1.79 mmol, 67% yield) as colorless oil. ¹H NMR (400 MHz, DMSO-d₆) δ=6.96-6.87 (m, 2H), 6.64 (ddd, J=2.4, 4.4, 8.4 Hz, 1H), 5.24 (s, 1H), 5.09 (s, 2H), 5.01-4.98 (m, 1H), 2.03 (s, 3H). MS (ESI) m/z 152.2 [M+H]⁺

To a mixture of 2-fluoro-5-(prop-1-en-2-yl)aniline (270 mg, 1.79 mmol, 1.00 eq) in methanol (10.0 mL) was added palladium on carbon (30.0 mg, 10% purity). The reaction mixture was stirred at 20° C. for 2 h under hydrogen atmosphere. The reaction mixture was filtered and concentrated to give 2-fluoro-5-isopropylaniline (270 mg, 1.76 mmol, 98% yield) as colorless oil. ¹H NMR (400 MHz, DMSO-d₆) δ=6.85 (dd, J=8.4, 11.6 Hz, 1H), 6.63 (dd, J=2.4, 8.8 Hz, 1H), 6.37 (ddd, J=2.4, 4.4, 8.2 Hz, 1H), 4.98 (s, 2H), 2.72 (quin, J=6.8 Hz, 1H), 1.15 (s, 3H), 1.13 (s, 3H). MS (ESI) m/z 154.0 [M+H]⁺

To a solution of 2-fluoro-5-isopropylaniline (270 mg, 1.79 mmol, 1.00 eq) and pyridine (278 mg, 3.52 mmol, 284 uL, 2.00 eq) in acetonitrile (10.0 mL) was added phenyl carbonochloridate (289 mg, 1.85 mmol, 231 uL, 1.05 eq). The reaction mixture was stirred at 20° C. for 2 h. The reaction mixture was poured into water (50.0 mL) and extracted with ethyl acetate (3×20.0 mL). The combined organic phase was separated, washed with brine (3×10.0 mL), dried over anhydrous sodium sulfate, filtered and concentrated to give phenyl (2-fluoro-5-isopropylphenyl)carbamate (350 mg, 1.28 mmol, 72% yield) as colorless oil. MS (ESI) m/z 274.1 [M+H]⁺

A mixture of 3-(6-(hydroxymethyl)-1-oxoisoindolin-2-yl)piperidine-2,6-dione 1 (80.0 mg, 291 umol, 1.00 eq) in dimethyl formamide (1.00 mL) was added sodium hydride (17.5 mg, 437 umol, 60% purity, 1.50 eq) and phenyl (2-fluoro-5-isopropylphenyl)carbamate (83.7 mg, 306 umol, 1.05 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 um; mobile phase: [water (0.225% Formic acid)-acetonitrile]; B %: 37%-67%, 10 min) and lyophilized to give (2-(2,6-dioxopiperidin-3-yl)-3-oxoisoindolin-5-yl)methyl (2-fluoro-5-isopropylphenyl)carbamate 405 (70.29 mg, 153 umol, 52% yield, 99% purity) as a white solid. ¹H NMR (400 MHz, DMSO-d₆) δ=10.99 (br s, 1H), 9.44 (br s, 1H), 7.81 (s, 1H), 7.71-7.61 (m, 2H), 7.51 (br d, J=7.2 Hz, 1H), 7.13 (dd, J=8.4, 10.6 Hz, 1H), 7.05-6.98 (m, 1H), 5.27 (s, 2H), 5.13 (dd, J=5.2, 13.2 Hz, 1H), 4.52-4.44 (m, 1H), 4.39-4.31 (m, 1H), 2.98-2.88 (m, 1H), 2.88-2.80 (m, 1H), 2.65-2.56 (m, 1H), 2.41 (dq, J=4.4, 13.2 Hz, 1H), 2.07-1.98 (m, 1H), 1.17 (d, J=6.8 Hz, 6H). MS (ESI) m/z 454.0 [M+H]⁺

Compound 406: To a solution of sulfuric acid (6.00 mL) was added nitric acid (8.40 g, 86.6 mmol, 6.00 mL, 65% purity, 3.90 eq) at −10° C. Then 2-(difluoromethoxy)-1,3-difluorobenzene (4.00 g, 22.2 mmol, 1.00 eq) was added slowly at −10° C. The mixture was stirred at −10° C. for 0.5 h. The reaction mixture was diluted with cold water (100 mL) and extracted with ethyl acetate (100 mL). The combined organic layer was washed with saturated sodium bicarbonate (100 mL), dried over anhydrous sodium sulfate and concentrated under vacuum. The residue was purified by column chromatography (SiO₂, Petroleum ether/Ethyl acetate=50/1 to 10/1) to give 2-(difluoromethoxy)-1,3-difluoro-5-nitrobenzene (170 mg, 755 umol, 3% yield) as yellow oil. ¹H NMR (400 MHz, CDCl₃) δ=8.02-7.90 (m, 2H), 6.71 (t, J=72.4 Hz, 1H).

To a solution of 2-(difluoromethoxy)-1,3-difluoro-5-nitrobenzene (170 mg, 755 umol, 1.00 eq) in the mixture of methanol (10.0 mL) and water (2.00 mL) was added iron powder (210 mg, 3.78 mmol, 5.00 eq) and ammonium chloride (323 mg, 6.04 mmol, 8.00 eq). The mixture was stirred at 80° C. for 1 h. The mixture was filtered to give a filter liquor, then was concentrated under reduced pressure to give a residue. The crude product was diluted with saturated sodium bicarbonate (30.0 mL) and exacted with ethyl acetate (30.0 mL). The organic phase was separated, washed with brine (10.0 mL), dried over sodium sulfate, filtered and concentrated under reduced pressure to give 4-(difluoromethoxy)-3,5-difluoroaniline (140 mg, 717 umol, 95% yield) as a yellow solid. MS (ESI) m/z 196.1 [M+H]⁺

To a solution of 4-(difluoromethoxy)-3,5-difluoroaniline (140 mg, 717 umol, 1.00 eq) in acetonitrile (10.0 mL) was added pyridine (283 mg, 3.59 mmol, 289 uL, 5.00 eq) and phenyl carbonochloridate (134 mg, 861 umol, 107 uL, 1.20 eq). The mixture was stirred at 25° C. for 0.5 h. The reaction mixture was concentrated under reduced pressure to give a residue. The crude product was purified by prep-TLC (SiO₂, Petroleum ether/Ethyl acetate=5/1) to give phenyl (4-(difluoromethoxy)-3,5-difluorophenyl)carbamate (180 mg, 571 umol, 79% yield) as a yellow solid. ¹H NMR (400 MHz, CDCl₃) δ=7.47-7.40 (m, 2H), 7.32-7.28 (m, 1H), 7.22-7.16 (m, 4H), 7.11 (br s, 1H), 6.57 (t, J=72.4 Hz, 1H). MS (ESI) m/z 316.0 [M+H]⁺

To a solution of phenyl (4-(difluoromethoxy)-3,5-difluorophenyl)carbamate (101 mg, 320 umol, 1.10 eq) in dimethyl formamide (2.00 mL) was added 3-(6-(hydroxymethyl)-1-oxoisoindolin-2-yl)piperidine-2,6-dione 1 (80.0 mg, 291 umol, 1.00 eq) and sodium hydride (23.3 mg, 583 umol, 60% purity, 2.00 eq) at 0° C. The mixture was stirred at 0° C. for 0.5 h. The reaction mixture was quenched with formic acid (0.500 ml) to give a solution. The solution was purified by prep-HPLC (column: Phenomenex Synergi C18 150*25 mm*10 um; mobile phase: [water (0.225% FA)-ACN]; B %: 33%-63%, 10 min) and lyophilized to give a residue. The crude product was dissolved in dimethyl formamide (2.00 mL) and purified by prep-HPLC (column: Phenomenex Synergi C18 150*25 mm*10 um; mobile phase: [water (0.225% FA)-ACN]; B %: 32%-65%, 11 min) and lyophilized to give a residue. The crude product was purified by column chromatography (SiO₂, Petroleum ether/Ethyl acetate=5/1 to 0/1) to give (2-(2,6-dioxopiperidin-3-yl)-3-oxoisoindolin-5-yl) methyl (4-(difluoromethoxy)-3,5-difluorophenyl)carbamate 406 (64.7 mg, 130 umol, 44% yield) as a white solid. ¹H NMR (400 MHz, DMSO-d₆) δ=11.01 (s, 1H), 10.34 (s, 1H), 7.80 (s, 1H), 7.71-7.67 (m, 1H), 7.66-7.62 (m, 1H), 7.32 (d, J=10.3 Hz, 2H), 7.17 (t, J=72.0 Hz, 1H), 5.30 (s, 2H), 5.13 (dd, J=5.1, 13.3 Hz, 1H), 4.52-4.43 (m, 1H), 4.40-4.29 (m, 1H), 2.97-2.86 (m, 1H), 2.63-2.56 (m, 1H), 2.40 (dq, J=4.3, 13.2 Hz, 1H), 2.05-1.97 (m, 1H). MS (ESI) m/z 496.1 [M+H]⁺

Compound 407: To a solution of 5-bromo-2-fluoroaniline (500 mg, 2.63 mmol, 1.00 eq), cyclopropylboronic acid (271 mg, 3.16 mmol, 1.20 eq) and tetrakis[triphenylphosphine]palladium(0) (304 mg, 263 umol, 0.100 eq) in dioxane (10.0 mL) was added cesium carbonate (2.57 g, 7.89 mmol, 3.00 eq), then evacuated with vacuum and back filled with nitrogen 3 times. The mixture was stirred at 100° C. for 12 h. The reaction mixture was filtered and concentrated to give a residue, which was purified by silica gel chromatography (petroleum ether/ethyl acetate=20/1 to 10/1) to afford 5-cyclopropyl-2-fluoroaniline (220 mg, 1.46 mmol, 55% yield) as colorless oil. ¹H NMR (400 MHz, DMSO-d₆) δ=6.81 (dd, J=8.4, 11.4 Hz, 1H), 6.45 (dd, J=2.4, 8.8 Hz, 1H), 6.22 (ddd, J=2.4, 4.4, 8.4 Hz, 1H), 4.98 (br s, 2H), 1.74 (tt, J=5.2, 8.4 Hz, 1H), 0.88-0.80 (m, 2H), 0.54-0.48 (m, 2H).

To a solution of 5-cyclopropyl-2-fluoroaniline (220 mg, 1.46 mmol, 1.00 eq) and pyridine (230 mg, 2.91 mmol, 234 uL, 2.00 eq) in acetonitrile (10.0 mL) was added phenyl carbonochloridate (239 mg, 1.53 mmol, 191 uL, 1.05 eq). The reaction mixture was stirred at 20° C. for 2 h. The reaction mixture was poured into water (50.0 mL) and extracted with ethyl acetate (3×20.0 mL). The combined organic phase was separated, washed with brine (3×10.0 mL), dried over anhydrous sodium sulfate, filtered and concentrated to give phenyl (5-cyclopropyl-2-fluorophenyl) carbamate (360 mg, 1.33 mmol, 91% yield) as a yellow solid. ¹H NMR (400 MHz, DMSO-d₆) δ=9.85 (br s, 1H), 7.45-7.41 (m, 2H), 7.29-7.24 (m, 1H), 7.21 (br d, J=7.8 Hz, 2H), 7.16-7.13 (m, 1H), 6.95-6.86 (m, 1H), 6.75 (d, J=7.8 Hz, 1H), 1.96-1.87 (m, 1H), 0.97-0.90 (m, 2H), 0.64-0.58 (m, 21H). MS (ESI) m/z 272.0 [M+H]⁺

A mixture of 3-(6-(hydroxymethyl)-1-oxoisoindolin-2-yl)piperidine-2,6-dione 1 (80.0 mg, 291 umol, 1.00 eq) in dimethyl formamide (1.00 mL) was added sodium hydride (17.5 mg, 437 umol, 60% purity, 1.50 eq) and phenyl (5-cyclopropyl-2-fluorophenyl) carbamate (83.0 mg, 306 umol, 1.05 eq) at 0° C. The reaction mixture was stirred at 20° C. for 1 h. The reaction mixture was added formic acid (2.00 mL) and filtered to give a filtrate, which was purified by Prep-HPLC (column: Phenomenex Luna C18 150*25 mm*10 um; mobile phase: [water (0.225% formic acid)-acetonitrile]; B %: 36%-66%, 10 min) and lyophilized to give (2-(2,6-dioxopiperidin-3-yl)-3-oxoisoindolin-5-yl)methyl (5-cyclopropyl-2-fluorophenyl) carbamate 407 (62.3 mg, 136 umol, 46% yield, 99% purity) as a white solid. ¹H NMR (400 MHz, DMSO-d₆) δ=10.98 (br s, 1H), 9.43 (br s, 1H), 7.80 (s, 1H), 7.73-7.59 (m, 2H), 7.35 (br d, J=7.2 Hz, 1H), 7.08 (dd, J=8.8, 10.4 Hz, 1H), 6.89-6.77 (m, 1H), 5.26 (s, 2H), 5.13 (dd, J=5.2, 13.2 Hz, 1H), 4.52-4.43 (m, 1H), 4.39-4.30 (m, 1H), 2.97-2.85 (m, 1H), 2.64-2.57 (m, 1H), 2.40 (dq, J=4.4, 13.2 Hz, 1H), 2.06-1.97 (m, 1H), 1.94-1.84 (m, 1H), 0.95-0.88 (m, 2H), 0.63-0.55 (m, 2H). MS (ESI) m/z 452.3 [M+H]⁺

Compound 408: A mixture of 5-bromo-2-fluoro-4-methyl-aniline (0.570 g, 2.79 mmol, 1.00 eq), 2,4,6-trimethyl-1,3,5,2,4,6-trioxatriborinane (1.40 g, 5.59 mmol, 1.56 mL, 50% purity, 2.00 eq), [1,1-Bis(diphenylphosphino)ferrocene]dichloropalladium (II) (102 mg, 140 umol, 0.05 eq), potassium phosphate (1.19 g, 5.59 mmol, 2.00 eq) in dioxane (10.0 mL) and water (0.500 mL) was degassed under vacuum and then purged with nitrogen for 3 times. The mixture was stirred at 90° C. for 12 h under nitrogen atmosphere. After filtration, the filtrate was concentrated under vacuum to give a residue. The residue was purified by silica gel column chromatography (petroleum ether, then petroleum ether/ethyl acetate=50/1) to afford 2-fluoro-4,5-dimethyl-aniline (0.350 g, 2.26 mmol, 81% yield, 90% purity) as light yellow solid. ¹H NMR (400 MHz, CDCl₃) δ=6.78 (d, J=12.0 Hz, 1H), 6.61 (d, J=8.8 Hz, 1H), 2.15 (s, 6H).

To a solution of 2-fluoro-4,5-dimethyl-aniline (0.200 g, 1.29 mmol, 90% purity, 1.00 eq) and pyridine (307 mg, 3.88 mmol, 313 uL, 3.00 eq) in acetonitrile (5.0 mL) was added phenyl carbonochloridate (223 mg, 1.42 mmol, 178 uL, 1.10 eq) at 0° C. The mixture was stirred at 25° C. for 12 h. The mixture was poured into water (20.0 mL) and extracted with ethyl acetate (3×10.0 mL). The organic phase was washed with hydrochloric acid solution (50.0 mL, 0.5 M), brine (50.0 mL), dried over anhydrous sodium sulfate, filtered and concentrated under reduced pressure to give a residue. The residue was purified by silica gel column chromatography (petroleum ether, then petroleum ether/ethyl acetate=50/1) to afford phenyl N-(2-fluoro-4,5-dimethyl-phenyl)carbamate (330 mg, 1.15 mmol, 89% yield, 90% purity) as light yellow solid. ¹H NMR (400 MHz, DMSO-d₆) δ=9.83-9.64 (m, 1H), 7.45-7.37 (m, 3H), 7.28-7.16 (m, 3H), 7.06 (d, J=11.6 Hz, 1H), 2.19 (s, 3H), 2.17 (s, 3H).

To a solution of 3-[6-(hydroxymethyl)-1-oxo-isoindolin-2-yl]piperidine-2,6-dione (105 mg, 364 umol, 95% purity, 1.00 eq) in tetrahydrofuran (4.00 mL) was added sodium hydride (29.2 mg, 729 umol, 60% purity, 2.00 eq) at 0° C., and then phenyl N-(2-fluoro-4,5-dimethyl-phenyl)carbamate (105 mg, 365 umol, 90% purity, 1.00 eq) was added. The mixture was stirred at 25° C. for 1 h. The mixture was poured into saturated ammonium chloride aqueous solution (20.0 mL) and extracted with ethyl acetate (3×10.0 mL). The organic phase was washed with saturated calcium chloride solution (20.0 ml), brine (20.0 mL), dried over anhydrous sodium sulfate, filtered and concentrated under reduced pressure to give a residue. The residue was purified by prep-HPLC (column: Phenomenex Synergi C18 150*25 mm*10 um; mobile phase: [water (0.225% FA)-ACN]; B %: 31%-61%, 10 min) and then lyophilized to afford (2-(2,6-dioxopiperidin-3-yl)-3-oxoisoindolin-5-yl)methyl (2-fluoro-4,5-dimethylphenyl)carbamate 408 (41.54 mg, 93.6 umol, 26% yield, 99% purity) was obtained as a white solid ¹H NMR (400 MHz, DMSO-d₆) δ=10.99 (s, 1H), 9.31 (s, 1H), 7.78 (s, 1H), 7.71-7.59 (m, 2H), 7.33 (d, J=8.0 Hz, 1H), 7.01 (d, J=11.6 Hz, 1H), 5.24 (s, 2H), 5.13 (dd, J=5.2, 13.2 Hz, 1H), 4.53-4.28 (m, 2H), 2.99-2.85 (m, 1H), 2.71-2.55 (m, 1H), 2.31-2.44 (m, 1H), 2.17 (s, 3H), 2.15 (s, 3H), 2.06-1.96 (m, 1H). MS (ESI) m/z 440.1 [M+H]⁺

Compound 409: To a solution of 1-(4-bromophenyl)-2,2,2-trifluoroethanone (5.00 g, 19.8 mmol, 3.01 mL, 1.00 eq) and tert-butyl carbamate (2.78 g, 23.7 mmol, 1.20 eq) and cesium carbonate (19.3 g, 59.3 mmol, 3.00 eq) in dioxane (100 mL) was added RuPhos Pd G3 (826.40 mg, 988.08 umol, 0.05 eq) under nitrogen. The mixture was stirred at 90° C. for 12 h. The mixture was diluted with water (150 mL) and extracted with ethyl acetate (3×80.0 mL). The combined organic layer was washed with brine (60.0 mL), dried over sodium sulfate, filtered and concentrated in vacuum. The residue was purified by silica gel chromatography (petroleum ether/ethyl acetate=5/1 to 2/1) to give tert-butyl (4-(2,2,2-trifluoroacetyl) phenyl)carbamate (2.60 g, 8.99 mmol, 45% yield) as a yellow solid. ¹H NMR (400 MHz, DMSO-d₆) δ=10.12 (s, 1H), 7.98 (d, J=8.2 Hz, 2H), 7.77-7.68 (m, 2H), 1.50 (s, 9H).

To a solution of tert-butyl (4-(2,2,2-trifluoroacetyl)phenyl)carbamate (2.60 g, 8.99 mmol, 1.00 eq) in methanol (30.0 mL) was added sodium borohydride (680 mg, 17.9 mmol, 2.00 eq) at 0° C. The mixture was stirred at 15° C. for 2 h. The mixture was poured into methanol (100 mL) and concentrated in vacuum. The residue was purified by silica gel chromatography (petroleum ether/ethyl acetate=4/1) to give tert-butyl (4-(2,2,2-trifluoro-1-hydroxyethyl)phenyl)carbamate (1.70 g, 5.84 mmol, 65% yield) as yellow solid. ¹H NMR (400 MHz, DMSO-d₆) δ=9.41 (s, 1H), 7.46 (d, J=8.7 Hz, 2H), 7.35 (d, J=8.6 Hz, 2H), 6.70 (d, J=5.6 Hz, 1H), 5.14-4.96 (m, 1H), 1.47 (s, 9H).

A solution of tert-butyl (4-(2,2,2-trifluoro-1-hydroxyethyl)phenyl)carbamate (1.50 g, 5.15 mmol, 1.00 eq) in dichloromethane (12.0 mL) and trifluoroacetic acid (4.00 mL) was stirred at 15° C. for 2 h. The mixture was concentrated in vacuum. The residue was purified by reverse phase chromatography (column: spherical C18, 20-45 um, 100 Å, SW 120, mobile phase: [water (0.1% Formic Acid)-ACN) and lyophilized to give 1-(4-aminophenyl)-2,2,2-trifluoroethanol (980 mg, 5.13 mmol, 99% yield) as yellow oil. ¹H NMR (400 MHz, DMSO-d₆) δ=7.09 (d, J=8.3 Hz, 2H), 6.54 (d, J=8.6 Hz, 2H), 6.44 (d, J=5.4 Hz, 1H), 5.16 (s, 2H), 4.91-4.80 (m, 1H).

To a mixture of 1-(4-aminophenyl)-2,2,2-trifluoroethanol (100 mg, 523 umol, 1.00 eq) and pyridine (207 mg, 2.62 mmol, 211 uL, 5.00 eq) in acetonitrile (2.00 mL) was added phenyl carbonochloridate (98.3 mg, 628 umol, 78.6 uL, 1.20 eq) dropwise at 0° C. The mixture was stirred at 15° C. for 2 h. The mixture was concentrated in vacuum. The residue was purified by silica gel chromatography (petroleum ether/ethyl acetate=10/1 to 3/1) to give phenyl (4-(2,2,2-trifluoro-1-hydroxyethyl)phenyl)carbamate (80.0 mg, 257 umol, 49% yield) as a white solid. ¹H NMR (400 MHz, DMSO-d₆) δ=10.31 (br s, 1H), 7.53 (br d, J=8.6 Hz, 2H), 7.46-7.40 (m, 4H), 7.28-7.20 (m, 3H), 6.77 (d, J=5.6 Hz, 1H), 5.09 (quin, J=6.8 Hz, 1H).

A solution of 3-(6-(hydroxymethyl)-1-oxoisoindolin-2-yl)piperidine-2,6-dione 1 (123 mg, 449 umol, 1.00 eq) in dimethyformamide (2.00 mL) was added triethylamine (137 mg, 1.35 mmol, 188 uL, 3.00 eq) in portions. The mixture was stirred at 0° C. for 20 min. Then the mixture was added phenyl phenyl (4-(2,2,2-trifluoro-1-hydroxyethyl)phenyl)carbamate (140 mg, 449 umol, 1.00 eq) and the mixture was stirred at 15° C. for 18 h. The mixture was quenched with 1 M hydrochloric acid (0.500 mL) and filtered. The filtrate was purified by prep-HPLC (column: YMC Triart 30*150 mm*7 um; mobile phase: [water (0.05% HCl)-ACN]; B %: 35%-55%, 7 min) and lyophilized to give (2-(2,6-dioxopiperidin-3-yl)-3-oxoisoindolin-5-yl)methyl (4-(2,2,2-trifluoro-1-hydroxyethyl)phenyl) carbamate 409 (19.62 mg, 37.1 umol, 8% yield, 93% purity) as an off-white solid. ¹H NMR (400 MHz, DMSO-d₆) δ=10.99 (s, 1H), 9.91 (s, 1H), 7.80 (s, 1H), 7.71-7.61 (m, 2H), 7.49 (br d, J=8.6 Hz, 2H), 7.42-7.36 (m, 2H), 6.87-6.63 (m, 1H), 5.28 (s, 2H), 5.13 (dd, J=5.1, 13.3 Hz, 1H), 5.06 (q, J=7.4 Hz, 1H), 4.52-4.42 (m, 1H), 4.40-4.30 (m, 1H), 2.98-2.85 (m, 1H), 2.60 (br d, J=17.9 Hz, 1H), 2.46-2.34 (m, 1H), 2.05-1.97 (m, 1H). MS (ESI) m/z 492.1 [M+H]⁺

Compound 410: To a solution of 5-bromo-2-fluoroaniline (500 mg, 2.63 mmol, 1.00 eq), ethylboronic acid (583 mg, 7.89 mmol, 3.00 eq) and tetrakis[triphenylphosphine]palladium(0) (304 mg, 263 umol, 0.100 eq) in dioxane (10.0 mL) was added cesium carbonate (2.57 g, 7.89 mmol, 3.00 eq), then evacuated with vacuum and back filled with nitrogen 3 times. The mixture was stirred at 90° C. for 12 h. The reaction mixture was filtered and concentrated to give a residue, which was purified by silica gel chromatography (petroleum ether/ethyl acetate=20/1 to 10/1) to afford 5-ethyl-2-fluoroaniline (220 mg, 1.55 mmol, 58% yield, 98% purity) as colorless oil. ¹H NMR (400 MHz, DMSO-d₆) δ=6.84 (dd, J=8.4, 11.6 Hz, 1H), 6.59 (dd, J=2.0, 8.8 Hz, 1H), 6.32 (ddd, J=2.2, 4.5, 8.0 Hz, 1H), 4.97 (s, 2H), 2.43 (q, J=7.6 Hz, 2H), 1.11 (t, J=7.6 Hz, 3H). MS (ESI) m/z 140.0 [M+H]⁺

To a solution of 5-ethyl-2-fluoroaniline (220 mg, 1.58 mmol, 1.00 eq) and pyridine (250 mg, 3.16 mmol, 255 uL, 2.00 eq) in acetonitrile (10.0 mL) was added phenyl carbonochloridate (259 mg, 1.66 mmol, 207 uL, 1.05 eq). The reaction mixture was stirred at 20° C. for 2 h. The reaction mixture was poured into water (50.0 mL) and extracted with ethyl acetate (3×20.0 mL). The combined organic phase was separated, washed with brine (3×10.0 mL), dried over anhydrous sodium sulfate, filtered and concentrated to give phenyl (5-ethyl-2-fluorophenyl)carbamate (350 mg, 1.35 mmol, 85% yield) as colorless oil. MS (ESI) m/z 260.1 [M+H]⁺

A mixture of 3-(6-(hydroxymethyl)-1-oxoisoindolin-2-yl)piperidine-2,6-dione 1 (80.0 mg, 291 umol, 1.00 eq) in dimethyl formamide (1.00 mL) was added sodium hydride (17.5 mg, 437 umol, 60% purity, 1.50 eq) and phenyl (5-ethyl-2-fluorophenyl)carbamate (79.4 mg, 306 umol, 1.05 eq) at 0° C. The reaction mixture was stirred at 20° C. for 1 h. The reaction mixture was added formic acid (2.00 mL) and filtered to give a filtrate, which was purified by Prep-HPLC (column: Phenomenex luna C18 150*25 mm*10 um; mobile phase: [water (0.225% formic acid)-acetonitrile]; B %: 34%-64%, 10 min) and lyophilized to give (2-(2,6-dioxopiperidin-3-yl)-3-oxoisoindolin-5-yl)methyl (5-ethyl-2-fluorophenyl)carbamate 410 (48.48 mg, 98.8 umol, 33% yield, 99% purity, formic acid) as a white solid. ¹H NMR (400 MHz, DMSO-d₆) δ=11.00 (s, 1H), 9.45 (br s, 1H), 7.81 (s, 1H), 7.71-7.66 (m, 1H), 7.66-7.62 (m, 1H), 7.48 (br d, J=7.6 Hz, 1H), 7.12 (dd, J=8.4, 10.8 Hz, 1H), 7.00-6.95 (m, 1H), 5.27 (s, 2H), 5.14 (dd, J=5.0, 13.2 Hz, 1H), 4.52-4.44 (m, 1H), 4.39-4.31 (m, 1H), 2.92 (ddd, J=5.5, 13.4, 17.6 Hz, 1H), 2.63-2.59 (m, 1H), 2.57 (d, J=7.6 Hz, 2H), 2.47-2.35 (m, 1H), 2.06-1.98 (m, 1H), 1.15 (t, J=7.6 Hz, 3H). MS (ESI) m/z 440.3 [M+H]⁺

Compound 411: To a solution of 1,3-difluoro-5-methyl-2-nitrobenzene (500 mg, 2.89 mmol, 1.00 eq) in ethanol (3.00 mL) and water (3.00 mL) was added iron (806 mg, 14.4 mmol, 5.00 eq) and ammonium chloride (772 mg, 14.4 mmol, 5.00 eq). After addition, the mixture was stirred at 80° C. for 1 h. The reaction mixture was filtered through celite, and then the filtrate was extracted with ethyl acetate (3×10.0 mL). The combined organic layers were washed with brine (10.0 mL), dried over sodium sulfate, filtered and the filtrate was concentrated in vacuum to give 2,6-difluoro-4-methylaniline (260 mg, 1.82 mmol, 62% yield) as brown oil. ¹H NMR (400 MHz, CDCl₃) δ=6.67-6.59 (m, 2H), 3.26 (s, 2H), 2.23 (s, 3H).

To a solution of 2,6-difluoro-4-methyl-aniline (260 mg, 1.82 mmol, 1.00 eq) in sulfuric acid (3.00 mL) was added N-chlorosuccinimide (333 mg, 2.50 mmol, 1.37 eq) at 0° C. After stirring the mixture at 60° C. for 2 h, the reaction was quenched by ice. The resulting mixture was adjust pH to 7 with sodium bicarbonate and extracted with ethyl acetate (3×10.0 mL). The combined organic layers were washed with brine (10.0 mL), dried over sodium sulfate, filtered and concentrated under vacuum to give product 3-chloro-2,6-difluoro-4-methyl-aniline (270 mg, 1.52 mmol, 83% yield) as a black solid. ¹H NMR (400 MHz, CDCl₃) δ=6.71 (d, J=10.8 Hz, 1H), 3.64 (s, 2H), 2.26 (s, 3H).

To a solution of 3-chloro-2,6-difluoro-4-methyl-aniline (50.0 mg, 281 umol, 1.00 eq) in tetrahydrofuran (1.00 mL) was added pyridine (44.5 mg, 563 umol, 2.00 eq) and phenyl carbonochloridate (52.9 mg, 337 umol, 1.20 eq). After addition, the mixture was stirred at 0° C. for 1 h. The reaction mixture was quenched by water (5.00 mL) and extracted with ethyl acetate (3×5.00 mL). The combined organic layers were washed with hydrochloric acid aqueous solution (5.00 mL, 0.5 M), saturated sodium bicarbonate aqueous solution (5.00 mL) and brine (5.00 mL). The separated organic layer was dried over sodium sulfate, filtered and concentrated under vacuum to give phenyl N-(3-chloro-2,6-difluoro-4-methyl-phenyl)carbamate (84 mg, crude) as brown oil.

To a solution of sodium hydride (16.9 mg, 423 umol, 60% purity, 1.50 eq) in N,N-dimethylformamide (1.00 mL) was added 3-[6-(hydroxymethyl)-1-oxo-isoindolin-2-yl]piperidine-2,6-dione (77.3 mg, 282 umol, 1.00 eq). The mixture was stirred at 0° C. under nitrogen atmosphere for 0.5 h, and hen phenyl N-(3-chloro-2,6-difluoro-4-methyl-phenyl)carbamate (84.0 mg, 282 umol, 1.00 eq) in N,N-dimethylformamide (0.500 mL) was added. After addition, the resulting mixture was stirred at 0° C. for 1 hour under nitrogen atmosphere. The reaction mixture was quenched with ammonium chloride solution (5.00 mL) at 0° C. and extracted with ethyl acetate (3×5.00 mL). The combined organic layers were washed with brine (5.00 mL), dried over sodium sulfate, filtered and concentrated under vacuum to give a residue. The residue was purified by prep-HPLC (FA condition, column: Phenomenex Synergi C18 150*25 mm*10 um; mobile phase: [water (0.225% FA)-ACN]; B %: 30%-60%, 10 min) and then lyophilized to give (2-(2,6-dioxopiperidin-3-yl)-3-oxoisoindolin-5-yl)methyl (3-chloro-2,6-difluoro-4-methylphenyl)carbamate 411 (38.5 mg, 80.0 umol, 28% yield, 99% purity) as an off-white solid. ¹H NMR (400 MHz, DMSO-d₆) δ=11.01 (s, 1H), 9.50 (s, 1H), 7.76 (s, 1H), 7.64 (s, 2H), 7.28 (d, J=9.2 Hz, 1H), 5.26 (s, 2H), 5.13 (dd, J=5.2, 13.2 Hz, 1H), 4.55-4.29 (m, 2H), 2.99-2.84 (m, 1H), 2.65-2.56 (m, 1H), 2.47-2.38 (m, 1H), 2.36 (s, 3H), 2.06-1.95 (m, 1H). MS (ESI) m/z 478.0 [M+H]⁺

Compound 412: To a solution of 2-bromo-6-fluorophenol (2.00 g, 10.5 mmol, 1.00 eq) in acetonitrile (20.0 mL) was added 1,2-dibromoethane (3.93 g, 20.9 mmol, 1.58 mL, 2.00 eq) and potassium carbonate (2.89 g, 20.9 mmol, 2.00 eq). The mixture was stirred at 50° C. for 4 h. The mixture was diluted with water (100 mL) and extracted with ethyl acetate (3×30.0 mL). The combined organic layers were washed with brine (50.0 mL), dried over sodium sulfate, filtered and concentrated under reduced pressure to give a residue. The residue was purified by column chromatography on silica gel (Petroleum ether/Ethyl acetate=1/0 to Petroleum ether/Ethylacetate=100/1) to afford 1-bromo-2-(2-bromoethoxy)-3-fluorobenzene (1.20 g, 4.03 mmol, 38% yield) as white oil. ¹H NMR (400 MHz, DMSO-d₆) δ=7.47 (td, J=1.5, 8.0 Hz, 1H), 7.34 (ddd, J=1.3, 8.4, 10.8 Hz, 1H), 7.17-7.07 (m, 1H), 4.37 (dd, J=5.0, 6.0 Hz, 2H), 3.84-3.73 (m, 2H).

To a solution of 1-bromo-2-(2-bromoethoxy)-3-fluorobenzene (1.40 g, 4.70 mmol, 1.00 eq) in tetrahydrofuran (20.0 mL) cooled to −78° C. was added n-butyllithium (2.5 M, 2.80 mL, 1.49 eq) under nitrogen atmosphere. The mixture was stirred at −78° C. for 2 h and then the mixture was gradually raised to 25° C., the reaction mixture was stirred at 25° C. for 1 h. The reaction solution was quenched with water (50.0 mL), then the reaction solution was extracted with ethyl acetate (3×20.0 mL). The combined organic phases was washed with brine (20.0 mL), dried over anhydrous sodium sulfate, and concentrated under reduced pressure to afford a residue. The residue was purified by column chromatography on silica gel (Petroleum ether/Ethyl acetate=1/0 to Petroleum ether/Ethyl acetate=5/1) to afford 7-fluoro-2,3-dihydrobenzofuran (370 mg, 2.68 mmol, 57% yield) as a yellow solid. ¹H NMR (400 MHz, DMSO-d₆) δ=7.08-6.95 (m, 2H), 6.80 (dt, J=4.5, 7.6 Hz, 1H), 4.61 (t, J=8.8 Hz, 2H), 3.24 (t, J=8.8 Hz, 2H).

To a solution of 7-fluoro-2,3-dihydrobenzofuran (310 mg, 2.24 mmol, 1.00 eq) was added to nitric acid (3.10 mL). The mixture was stirred at −10° C. for 2 h. The reaction mixture was poured into ice water (60.0 mL) slowly. The reaction solution was extracted with ethyl acetate (3×15.0 mL). The organic phases were combined, dried with anhydrous sodium sulfate, and concentrated under reduced pressure to give a residue. The residue was purified by column chromatography on silica gel (Petroleum ether/Ethyl acetate=1/0 to 5/1) to afford 7-fluoro-5-nitro-2,3-dihydrobenzofuran (210 mg, 1.15 mmol, 51% yield) as a brown solid. ¹H NMR (400 MHz, DMSO-d₆) δ=8.10-8.00 (m, 2H), 4.84 (t, J=8.8 Hz, 2H), 3.39-3.35 (m, 2H)

To a solution of 7-fluoro-5-nitro-2,3-dihydrobenzofuran (200 mg, 1.09 mmol, 1.00 eq) in ethyl acetate (5.00 mL) was added palladium on carbon (200 mg, 10% purity) under hydrogen atmosphere. The mixture was stirred at 25° C. for 12 h. The reaction mixture was filtered and the filtrate was concentrated under reduced pressure to afford 7-fluoro-2,3-dihydrobenzofuran-5-amine (120 mg, 784 umol, 72% yield,) as a white solid

To a solution of 7-fluoro-2,3-dihydrobenzofuran-5-amine (120 mg, 784 umol, 1.00 eq) in acetonitrile (2.00 mL) was added pyridine (186 mg, 2.35 mmol, 190 uL, 3.00 eq) and phenyl carbonochloridate (135 mg, 862 umol, 108 uL, 1.10 eq). The mixture was stirred at 25° C. for 4 h. The reaction mixture was concentrated under reduced pressure to afford a residue. The residue was purified by reverse-phase HPLC (column: spherical C18, 20-45 um, 100 Å, SW 120, mobile phase:[water (0.1% formic acid)-acetonitrile]) and the desired eluent was lyophilized to afford phenyl (7-fluoro-2,3-dihydrobenzofuran-5-yl)carbamate (120 mg, 422 umol, 54% yield, 96% purity) as a white solid. ¹H NMR (400 MHz, DMSO-d₆) δ=10.21 (br s, 1H), 7.47-7.38 (m, 2H), 7.25-7.15 (m, 4H), 6.79-6.72 (m, 1H), 4.59 (t, J=8.8 Hz, 2H), 3.23 (t, J=8.8 Hz, 2H).

To a solution of phenyl (7-fluoro-2,3-dihydrobenzofuran-5-yl)carbamate (80.0 mg, 293 umol, 1.00 eq) in dimethyl formamide (300 uL) was added sodium hydride (23.4 mg, 586 umol, 60% purity, 2.00 eq) and 3-(6-(hydroxymethyl)-1-oxoisoindolin-2-yl)piperidine-2,6-dione 1 (88.3 mg, 322 umol, 1.10 eq). The mixture was stirred at 25° C. for 1 h. The pH of the mixture was adjusted to 7 with formic acid, then the mixture was diluted with dimethyl formamide (1.00 mL). The residue was purified by Prep-HPLC (column: Phenomenex luna C18 150*25 mm*10 um; mobile phase: [water (0.225% formic acid)-acetonitrile]; B %: 24%-54%, 10 min) and the desired eluent was lyophilized to afford (2-(2,6-dioxopiperidin-3-yl)-3-oxoisoindolin-5-yl)methyl (7-fluoro-2,3-dihydrobenzofuran-5-yl)carbamate 412 (40.42 mg, 80.1 umol, 27% yield, 99% purity, formic acid) as a white solid. ¹H NMR (400 MHz, DMSO-d₆) δ=10.99 (br s, 1H), 9.77 (br s, 1H), 7.78 (s, 1H), 7.71-7.60 (m, 2H), 7.24-7.10 (m, 2H), 5.25 (s, 2H), 5.12 (dd, J=5.4, 13.2 Hz, 1H), 4.57 (t, J=8.8 Hz, 2H), 4.51-4.43 (m, 1H), 4.40-4.30 (m, 1H), 3.21 (t, J=8.8 Hz, 2H), 2.91 (ddd, J=5.4, 13.6, 17.6 Hz, 1H), 2.63-2.58 (m, 1H), 2.46-2.34 (m, 1H), 2.06-1.96 (m, 1H). MS (ESI) m/z 454.1 [M+H]⁺

Compound 413: To a solution of 2,5-difluoro-4-methylaniline (300 mg, 2.10 mmol, 1.00 eq) and pyridine (498 mg, 6.29 mmol, 508 uL, 3.00 eq) in acetonitrile (5.00 mL) was added phenyl carbonochloridate (345 mg, 2.20 mmol, 276 uL, 1.05 eq) dropwise at 0° C. After addition, the resulting mixture was stirred at 25° C. for 2 h. The mixture was poured into water (30.0 mL) and extracted with ethyl acetate (3×10.0 mL). The organic phase was washed with brine (50.0 mL), dried over anhydrous sodium sulfate, filtered and concentrated under reduced pressure to give a residue. The residue was purified by silica gel column chromatography (petroleum ether, then petroleum ether/ethyl acetate=5/1) to afford phenyl (2,5-difluoro-4-methylphenyl)carbamate (370 mg, 1.12 mmol, 54% yield, 80% purity) as a light yellow solid. ¹H NMR (400 MHz, DMSO-d₆) δ=10.07 (s, 1H), 7.46-7.39 (m, 2H), 7.29-7.20 (m, 4H), 6.78-6.73 (m, 1H), 2.20 (s, 3H)

To a solution of 3-(6-(hydroxymethyl)-1-oxoisoindolin-2-yl)piperidine-2,6-dione 1 (39.5 mg, 137 umol, 95% purity, 0.90 eq) in N,N-dimethylformamide (2.00 mL) was added sodium hydride (12.2 mg, 304 umol, 60% purity, 2.00 eq) at 0° C., then phenyl (2,5-difluoro-4-methylphenyl)carbamate (50.0 mg, 152 umol, 80% purity, 1.00 eq) was added. The mixture was stirred at 25° C. for 1 h. The mixture was poured into saturated ammonium chloride aqueous solution (30.0 mL) and extracted with ethyl acetate (3×20.0 mL). The organic phase was washed with saturated calcium chloride aqueous solution (20.0 ml), brine (20.0 mL), dried over anhydrous sodium sulfate, filtered and concentrated under reduced pressure to give a residue. The residue was purified by prep-HPLC (FA condition; column: Phenomenex Gemini-NX C18 75*30 mm*3 um; mobile phase: [water (0.225% FA)-ACN]; B %: 32%-62%, 7 min) and then lyophilized to afford (2-(2,6-dioxopiperidin-3-yl)-3-oxo isoindolin-5-yl)methyl (2,5-difluoro-4-methylphenyl)carbamate 413 (51.13 mg, 114 umol, 75% yield, 99% purity) as a white solid. ¹H NMR (400 MHz, DMSO-d₆) δ=10.99 (s, 1H), 9.67 (s, 1H), 7.81 (s, 1H), 7.70-7.61 (m, 2H), 7.55-7.48 (m, 1H), 7.19 (dd, J=6.8, 10.8 Hz, 1H), 5.28 (s, 2H), 5.13 (dd, J=4.0, 13.2 Hz, 1H), 4.52-4.30 (m, 2H), 2.98-2.84 (m, 1H), 2.70-2.56 (m, 1H), 2.44-2.32 (m, 1H), 2.18 (s, 3H), 2.07-1.95 (m, 1H). MS (ESI) m/z 444.1 [M+H]⁺

Compound 414: To a solution of 3-(difluoro(4-methoxyphenyl)methyl)bicyclo[1.1.1]pentan-1-amine (100 mg, 363 umol, 1.00 eq, hydrochloride) and pyridine (143 mg, 1.81 mmol, 146 uL, 5.00 eq) in acetonitrile (1.00 mL) was added phenyl carbonochloridate (170 mg, 1.09 mmol, 136 uL, 3.00 eq) at 0° C. The mixture was stirred at 20° C. for 1 h. The reaction mixture was quenched with water (15 mL), extracted with ethyl acetate (3×20 mL). The organic layers were washed with brine (20 mL), dried over anhydrous sodium sulfate, filtered and concentrated to afford a residue. The residue was purified by reversed phase (C18, 40 g; condition: water/acetonitrile=1/0 to 0/1, 0.1% formic acid) and lyophilized to afford phenyl (3-(difluoro(4-methoxyphenyl)methyl)bicyclo[1.1.1]pentan-1-yl)carbamate (100 mg, 278 umol, 77% yield) as a white solid. ¹H NMR (400 MHz, DMSO-d₆) δ=8.59 (s, 1H), 7.38-7.32 (m, 4H), 7.23-7.17 (m, 1H), 7.08 (d, J=7.7 Hz, 2H), 7.02 (d, J=8.5 Hz, 2H), 3.79 (s, 3H), 1.99 (s, 6H).

To a solution of 3-(6-(hydroxymethyl)-1-oxoisoindolin-2-yl)piperidine-2,6-dione 1 (70.0 mg, 255 umol, 1.00 eq) and phenyl (3-(difluoro(4-methoxyphenyl)methyl)bicyclo[1.1.1]pentan-1-yl)carbamate (91.7 mg, 255 umol, 1.00 eq) in dimethylformamide (1.00 mL) was added sodium hydride (12.3 mg, 306 umol, 60% purity, 1.20 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 um; mobile phase: [water (0.225% formic acid)-acetonitrile]; B %: 37%-67%, 10 min) and lyophilized to afford (2-(2,6-dioxopiperidin-3-yl)-3-oxoisoindolin-5-yl)methyl(3-(difluoro(4-methoxyphenyl)methyl) bicyclo[1.1.1]pentan-1-yl)carbamate 414 (41.76 mg, 76.6 umol, 30% yield, 99% purity) as a white solid. ¹H NMR (400 MHz, DMSO-d₆) δ=10.98 (br s, 1H), 8.15 (br s, 1H), 7.68 (s, 1H), 7.62-7.56 (m, 2H), 7.32 (br d, J=8.5 Hz, 2H), 7.02 (d, J=8.7 Hz, 2H), 5.14-5.07 (m, 3H), 4.49-4.42 (m, 1H), 4.36-4.28 (m, 1H), 3.79 (s, 3H), 2.96-2.86 (m, 1H), 2.63-2.57 (m, 1H), 2.45-2.37 (m, 1H), 2.03-1.98 (m, 1H), 1.94 (s, 6H). MS (ESI) m/z 520.2 [M−19]⁺

Compound 415: To a solution of 3-morpholinobicyclo[1.1.1]pentan-1-amine (100 mg, 489 umol, 1.00 eq, hydrochloride) and cesium carbonate (478 mg, 1.47 mmol, 3.00 eq) in acetonitrile (1.00 mL) was added phenyl carbonochloridate (229 mg, 1.47 mmol, 184 uL, 3.00 eq) at 0° C. The mixture was stirred at 20° C. for 1 h. The mixture was quenched by ice water (20 mL), extracted with ethyl acetate (3×40 mL). The organic layers were washed with brine (30 mL), dried over anhydrous sodium sulfate, filtered and concentrated to afford a residue. The residue was purified by reversed phase (C18, 40 g; condition: water/acetonitrile=1/0 to 0/1, 0.1% formic acid) and lyophilized to afford phenyl (3-morpholinobicyclo [1.1.1]pentan-1-yl) carbamate (100 mg, 347 umol, 71% yield) as a white solid. ¹H NMR (400 MHz, DMSO-d₆) δ=8.47 (s, 1H), 7.41-7.32 (m, 2H), 7.24-7.16 (m, 1H), 7.09 (br d, J=7.8 Hz, 2H), 3.59-3.54 (m, 4H), 2.37-2.30 (m, 4H), 1.92 (s, 6H).

To a solution of 3-(6-(hydroxymethyl)-1-oxoisoindolin-2-yl)piperidine-2,6-dione 1 (80.0 mg, 292 umol, 1.00 eq) and phenyl (3-morpholinobicyclo[1.1.1]pentan-1-yl)carbamate (92.5 mg, 321 umol, 1.10 eq) in dimethylformamide (1.00 mL) was added sodium hydride (17.5 mg, 438 umol, 60% purity, 1.50 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 um; mobile phase: [water (0.225% formic acid)-acetonitrile]; B %: 1%-29%, 10 min) and lyophilized to afford (2-(2,6-dioxopiperidin-3-yl)-3-oxoisoindolin-5-yl)methyl (3-morpholinobicyclo[1.1.1]pentan-1-yl)carbamate 415 (82.42 mg, 174 umol, 60% yield, 99% purity) as a white solid. ¹H NMR (400 MHz, DMSO-d₆) δ=10.99 (s, 1H), 8.03 (br s, 1H), 7.70 (s, 1H), 7.60 (s, 2H), 5.16-5.08 (m, 3H), 4.50-4.42 (m, 1H), 4.36-4.30 (m, 1H), 3.58-3.53 (m, 4H), 2.96-2.86 (m, 1H), 2.64-2.57 (m, 1H), 2.40 (br dd, J=4.4, 13.2 Hz, 1H), 2.31 (br s, 4H), 2.04-1.97 (m, 1H), 1.87 (s, 6H). MS (ESI) m/z 469.2 [M+H]⁺

Compound 416: To a solution of 3-cyclopropylaniline (200 mg, 1.50 mmol, 1.00 eq) in acetonitrile (2.00 mL) was added phenyl carbonochloridate (247 mg, 1.58 mmol, 197 uL, 1.05 eq) and pyridine (356 mg, 4.50 mmol, 364 uL, 3.00 eq). The mixture was stirred at 25° C. for 2 h. The reaction mixture was concentrated under reduced pressure to afford a residue. The residue was diluted with water (30.0 mL) and extracted with ethyl acetate (3×10.0 mL). The combined organic layers were washed with brine (10.0 mL), dried over sodium sulfate, filtered and concentrated under reduced pressure to give a residue. The residue was purified by column chromatography (SiO₂, Petroleum ether/Ethyl acetate=1/0 to 5/1) to afford phenyl (3-cyclopropylphenyl)carbamate (353 mg, 1.39 mmol, 93% yield) as white oil. ¹H NMR (400 MHz, DMSO-d₆) δ=10.13 (br s, 1H), 7.46-7.41 (m, 2H), 7.30-7.15 (m, 6H), 6.77 (d, J=7.6 Hz, 1H), 1.92-1.84 (m, 1H), 0.98-0.91 (m, 2H), 0.62 (q, J=5.2 Hz, 2H).

To a solution of phenyl (3-cyclopropylphenyl)carbamate (81.3 mg, 321 umol, 1.10 eq) in dimethyl formamide (500 uL) was added sodium hydride (23.3 mg, 583 umol, 60% purity, 2.00 eq) and 3-(6-(hydroxymethyl)-1-oxoisoindolin-2-yl)piperidine-2,6-dione 1 (80.0 mg, 292 umol, 1.00 eq). The mixture was stirred at 25° C. for 1 h. The pH of the mixture was adjusted to 7 with formic acid, then the mixture was diluted with dimethyl formamide (1.00 mL). The residue was purified by Prep-HPLC (column: Phenomenex luna C18 150*25 mm*10 um; mobile phase: [water (0.225% formic acid)-acetonitrile]; B %: 32%-62%, 10 min) and the desired eluent was lyophilized to afford (2-(2,6-dioxopiperidin-3-yl)-3-oxoisoindolin-5-yl)methyl (3-cyclopropylphenyl)carbamate 416 (58.73 mg, 139 umol, 48% yield, 99% purity) as a white solid. ¹H NMR (400 MHz, DMSO-d₆) δ=10.99 (s, 1H), 9.70 (s, 1H), 7.79 (s, 1H), 7.70-7.61 (m, 2H), 7.26-7.18 (m, 2H), 7.17-7.10 (m, 1H), 6.71 (d, J=7.6 Hz, 1H), 5.26 (s, 2H), 5.13 (dd, J=5.1, 13.2 Hz, 1H), 4.50-4.42 (m, 1H), 4.40-4.28 (m, 1H), 2.97-2.85 (m, 1H), 2.60 (br dd, J=1.9, 15.6 Hz, 1H), 2.40 (dq, J=4.3, 13.2 Hz, 1H), 2.06-1.96 (m, 1H), 1.90-1.80 (m, 1H), 0.96-0.88 (m, 2H), 0.60 (dd, J=2.0, 5.2 Hz, 2H). MS (ESI) m/z 434.0 [M+H]⁺

Compound 417: To a solution of 3-isopropylaniline (200 mg, 1.48 mmol, 208 uL, 1.00 eq) in acetonitrile (2.00 mL) was added phenyl carbonochloridate (243 mg, 1.55 mmol, 195 uL, 1.05 eq) and pyridine (351 mg, 4.44 mmol, 358 uL, 3.00 eq). The mixture was stirred at 25° C. for 2 h. The reaction mixture was concentrated under reduced pressure to afford a residue. The residue was diluted with water (30.0 mL) and extracted with ethyl acetate (3×10.0 mL). The combined organic layers were washed with brine (10.0 mL), dried over sodium sulfate, filtered and concentrated under reduced pressure to give a residue. The residue was purified by column chromatography (SiO₂, Petroleum ether/Ethyl acetate=1/0 to 5/1) to afford phenyl (3-isopropylphenyl)carbamate (357 mg, 1.38 mmol, 94% yield, 99% purity) as pink oil. ¹H NMR (400 MHz, DMSO-d₆) δ=10.15 (br s, 1H), 7.48-7.39 (m, 3H), 7.35-7.17 (m, 5H), 6.94 (d, J=7.6 Hz, 1H), 2.85 (spt, J=6.8 Hz, 1H), 1.19 (d, J=7.2 Hz, 6H).

To a solution of phenyl (3-isopropylphenyl)carbamate (81.9 mg, 321 umol, 1.10 eq) in dimethyl formamide (500 uL) was added sodium hydride (23.3 mg, 583 umol, 60% purity, 2.00 eq) and 3-(6-(hydroxymethyl)-1-oxoisoindolin-2-yl)piperidine-2,6-dione 1 (80.0 mg, 292 umol, 1.00 eq). The mixture was stirred at 25° C. for 1 h. The pH of the mixture was adjusted to 7 with formic acid, then the mixture was diluted with dimethyl formamide (1.00 mL). The residue was purified by Prep-HPLC (column: Phenomenex luna C18 150*25 mm*10 um; mobile phase: [water (0.225% formic acid)-acetonitrile]; B %: 35%-65%, 10 min) and the desired eluent was lyophilized to afford (2-(2,6-dioxopiperidin-3-yl)-3-oxoisoindolin-5-yl)methyl (3-isopropylphenyl)carbamate 417 (84.17 mg, 191 umol, 66% yield, 99% purity) as a white solid. ¹H NMR (400 MHz, DMSO-d₆) δ=10.99 (br s, 1H), 9.72 (s, 1H), 7.79 (s, 1H), 7.71-7.60 (m, 2H), 7.37 (s, 1H), 7.28 (br d, J=8.0 Hz, 1H), 7.22-7.15 (m, 1H), 6.88 (d, J=7.6 Hz, 1H), 5.26 (s, 2H), 5.13 (dd, J=5.2, 13.2 Hz, 1H), 4.51-4.43 (m, 1H), 4.38-4.30 (m, 1H), 2.98-2.86 (m, 1H), 2.82 (td, J=6.9, 13.6 Hz, 1H), 2.60 (td, J=2.1, 15.2 Hz, 1H), 2.46-2.33 (m, 1H), 2.06-1.96 (m, 1H), 1.17 (d, J=6.8 Hz, 6H). MS (ESI) m/z 436.0 [M+H]⁺

Compound 418: To a solution of 2, 4-difluoro-5-nitrophenol (3.00 g, 17.1 mmol, 1.00 eq) in acetonitrile (60.0 mL) was added potassium hydroxide (2.88 g, 51.4 mmol, 3.00 eq) at 0° C. After addition, the mixture was stirred at 0° C. for 30 min. Then diethyl (2-bromo-2,2-difluoroethyl)phosphonate (14.5 g, 51.4 mmol, 3.00 eq) was added to the mixture dropwise at 0° C. After stirring at 20° C. for 3 h, the resulting mixture was poured into ammonium chloride aqueous solution (100 mL) and extracted with ethyl acetate (3×50.0 mL). The organic layer was washed with brine, dried over anhydrous sodium sulfate and filtered. The filtrate was concentrated under vacuum to give a residue. The residue was purified by silica gel column chromatography (petroleum ether) to give 1-(difluoromethoxy)-2,4-difluoro-5-nitrobenzene (1.90 g, 8.02 mmol, 47% yield, 95% purity) as colourless oil. ¹H NMR (400 MHz, DMSO-d₆) δ=8.30 (t, J=8.0 Hz, 1H), 8.02 (t, J=10.8 Hz, 1H), 7.39 (t, J=72.4 Hz, 1H).

To a solution of 1-(difluoromethoxy)-2,4-difluoro-5-nitrobenzene (1.90 g, 8.02 mmol, 95% purity, 1.00 eq) in methanol (20.0 mL) was added palladium on carbon (190 mg, 10% purity) under nitrogen atmosphere. The suspension was degassed under vacuum and purged with hydrogen for 3 times. After stirring at 20° C. for 16 h under hydrogen atmosphere (15 Psi), the mixture was filtered. The filter cake was washed with ethyl acetate (20.0 mL). The filtrate was concentrated under vacuum to give 5-(difluoromethoxy)-2,4-difluoroaniline (1.30 g, 6.33 mmol, 79% yield, 95% purity) as brown oil. It was used for next step directly without purification. ¹H NMR (400 MHz, DMSO-d₆) δ=7.26 (d, J=11.6 Hz, 1H), 7.09 (t, J=73.2 Hz, 1H), 6.73 (t, J=8.4 Hz, 1H), 5.27 (s, 2H).

To a solution of 5-(difluoromethoxy)-2,4-difluoroaniline (800 mg, 3.90 mmol, 95% purity, 1.00 eq) and pyridine (462 mg, 5.84 mmol, 1.50 eq) in acetonitrile (8.00 mL) was added phenyl carbonochloridate (640 mg, 4.09 mmol, 1.05 eq) at 0° C. After addition, the mixture was stirred at 20° C. for 5 h. The mixture was poured into water (50 mL) and extracted with ethyl acetate (3×50 mL). The separated organic layer was washed with brine, dried over anhydrous sodium sulfate and filtered. The filtrate was concentrated under vacuum to give a residue. The residue was purified by silica gel column chromatography (petroleum ether/ethyl acetate=100/1 to 20/1) to give phenyl (5-(difluoromethoxy)-2,4-difluorophenyl)carbamate (1.20 g, 3.72 mmol, 95% yield, 97% purity) as colourless oil. MS (ESI) m/z 316.1 [M+H]⁺

To a mixture of 3-(6-(hydroxymethyl)-1-oxoisoindolin-2-yl)piperidine-2,6-dione 1 (47.9 mg, 174 umol, 1.10 eq) in tetrahydrofuran (1.00 mL) was added sodium hydride (7.61 mg, 190 umol, 60% purity, 1.20 eq) at 0° C. After stirring at 0° C. for 15 min, phenyl (5-(difluoromethoxy)-2,4-difluorophenyl)carbamate (50.0 mg, 159 umol, 1.00 eq) was added to the mixture. The resulting mixture was stirred at 20° C. for 1 h. The mixture was poured into saturated ammonium chloride aqueous solution (5 mL) and extracted with ethyl acetate (3×10 mL). The organic layer was washed with brine, dried over anhydrous sodium sulfate and filtered. The filtrate was concentrated under vacuum to give a residue. The residue was purified by Prep-HPLC (column: Phenomenex Gemini-NX C18 75*30 mm*3 um; mobile phase: [water (0.225% FA)-ACN]; B %: 30%-60%, 7 min) and lyophilized to give (2-(2,6-dioxopiperidin-3-yl)-3-oxoisoindolin-5-yl)methyl(5-(difluoromethoxy)-2,4-difluorophenyl) carbamate 418 (47.89 mg, 96.2 umol, 61% yield, 99% purity) as a white solid. ¹H NMR (400 MHz, DMSO-d₆) δ=11.00 (s, 1H), 9.76 (s, 1H), 7.81 (s, 1H), 7.75 (t, J=8.4 Hz, 1H), 7.71-7.62 (m, 2H), 7.58 (t, J=6.8 Hz, 1H), 7.20 (t, J=73.2 Hz, 1H), 5.29 (s, 2H), 5.13 (dd, J=5.2, 13.2 Hz, 1H), 4.52-4.44 (m, 1H), 4.40-4.31 (m, 1H), 2.98-2.85 (m, 1H), 2.65-2.57 (m, 1H), 2.47-2.35 (m, 1H), 2.07-1.96 (m, 1H). MS (ESI) m/z 496.1 [M+H]⁺

Compound 419: To a solution of 3-(difluoromethyl)bicyclo[1.1.1]pentan-1-amine (100 mg, 590 umol, 1.00 eq, hydrochloride) and pyridine (233 mg, 2.95 mmol, 238 uL, 5.00 eq) in acetonitrile (1.00 mL) was added phenyl carbonochloridate (277 mg, 1.77 mmol, 222 uL, 3.00 eq) at 0° C. The mixture was stirred at 20° C. for 1 h. The mixture was quenched by ice water (20 mL), extracted with ethyl acetate (3×40 mL). The organic layers were washed with brine (30 mL), dried over anhydrous sodium sulfate, filtered and concentrated to afford a crude product. The crude product was purified by prep-NPLC (column: Welch Ultimate XB-CN 250*70*10 um; mobile phase: [Heptane-ethyl alcohol (0.1% ammonium hydroxide)]; B %: 1%-40%, 15 min) and concentrated to afford phenyl (3-(difluoromethyl)bicyclo[1.1.1]pentan-1-yl)carbamate (100 mg, 395 umol, 67% yield) as a white solid. ¹H NMR (400 MHz, DMSO-d₆) δ=8.60 (br s, 1H), 7.40-7.35 (m, 2H), 7.24-7.19 (m, 1H), 7.10 (br d, J=7.8 Hz, 2H), 6.17 (br t, J=56.4 Hz, 1H), 2.06 (s, 6H).

To a solution of 3-(6-(hydroxymethyl)-1-oxoisoindolin-2-yl)piperidine-2,6-dione 1 (80.0 mg, 292 umol, 1.00 eq) and phenyl (3-(difluoromethyl)bicyclo[1.1.1]pentan-1-yl)carbamate (81.3 mg, 321 umol, 1.10 eq) in dimethylformamide (1.00 mL) was added sodium hydride (17.5 mg, 438 umol, 60% purity, 1.50 eq) at 0° C. The mixture was stirred at 20° C. for 1 h. The mixture was quenched by formic acid (1 mL) and filtered. The filtrate was purified by prep-HPLC (column: Phenomenex Synergi C18 150*25 mm*10 um; mobile phase: [water (0.225% formic acid)-acetonitrile]; B %: 23%-53%, 10 min) and lyophilized to afford a residue. The residue was further purified by prep-HPLC (column: Phenomenex luna C18 150*25 mm*10 um; mobile phase: [water (0.225% formic acid)-acetonitrile]; B %: 22%-52%, 10 min) and lyophilized to afford (2-(2,6-dioxopiperidin-3-yl)-3-oxoisoindolin-5-yl)methyl (3-(difluoromethyl)bicyclo[1.1.1]pentan-1-yl) carbamate 419 (57.33 mg, 131 umol, 45% yield, 99% purity) as a white solid. ¹H NMR (400 MHz, DMSO-d₆) δ=11.00 (br s, 1H), 8.18 (br s, 1H), 7.71 (s, 1H), 7.61 (s, 2H), 6.16 (brt, J=56.4 Hz, 1H), 5.12 (m, 3H), 4.46 (m, 1H), 4.34 (m, 1H), 2.91 (m, 1H), 2.60 (m, 1H), 2.53 (m, 1H), 2.41 (m, 1H), 2.01 (s, 6H). MS (ESI) m/z 434.1 [M+H]⁺

Compound 420: To a solution of 3-ethylaniline (200 mg, 1.65 mmol, 206 uL, 1.00 eq) in acetonitrile (2.00 mL) was added pyridine (392 mg, 4.95 mmol, 400 uL, 3.00 eq) and phenyl carbonochloridate (271 mg, 1.73 mmol, 217 uL, 1.05 eq). The mixture was stirred at 25° C. for 2 h. The reaction mixture was concentrated under reduced pressure to afford a residue. The residue was diluted with water (30.0 mL) and extracted with ethyl acetate (3×10.0 mL). The combined organic layers were washed with brine (10.0 mL), dried over, filtered and concentrated under reduced pressure to give a residue. The residue was purified by column chromatography (SiO2, Petroleum ether/Ethyl acetate=1/0 to 9/1) to afford phenyl (3-ethylphenyl)carbamate (369 mg, 1.53 mmol, 93% yield) as yellow oil. ¹H NMR (400 MHz, DMSO-d₆) δ=10.15 (br s, 1H), 7.46-7.37 (m, 3H), 7.33 (br d, J=8.4 Hz, 1H), 7.29-7.19 (m, 4H), 6.91 (d, J=7.6 Hz, 1H), 2.58 (q, J=7.6 Hz, 2H), 1.20-1.15 (m, 3H).

To a solution of phenyl (3-ethylphenyl)carbamate (77.4 mg, 321 umol, 1.10 eq) in dimethyl formamide (500 uL) was added sodium hydride (23.3 mg, 583 umol, 60% purity, 2.00 eq) and 3-(6-(hydroxymethyl)-1-oxoisoindolin-2-yl)piperidine-2,6-dione 1 (80 mg, 291.68 umol, 1 eq). The mixture was stirred at 25° C. for 1 h. The pH of the mixture was adjusted to 7 with formic acid, then the mixture was diluted with dimethyl formamide (1.00 mL). The residue was purified by Prep-HPLC (column: Phenomenex luna C18 150*25 mm*10 um; mobile phase: [water (0.225% formic acid)-acetonitrile]; B %: 32%-62%, 10 min) and the desired eluent was lyophilized to afford (2-(2,6-dioxopiperidin-3-yl)-3-oxoisoindolin-5-yl)methyl (3-ethylphenyl)carbamate 420 (48.65 mg, 114 umol, 39% yield, 99% purity) as a white solid. ¹H NMR (400 MHz, DMSO-d₆) δ=11.00 (br s, 1H), 9.74 (s, 1H), 7.80 (s, 1H), 7.71-7.61 (m, 2H), 7.34 (s, 1H), 7.28 (br d, J=8.0 Hz, 1H), 7.21-7.14 (m, 1H), 6.85 (d, J=7.6 Hz, 1H), 5.27 (s, 2H), 5.13 (dd, J=5.1, 13.2 Hz, 1H), 4.53-4.44 (m, 1H), 4.39-4.29 (m, 1H), 2.97-2.87 (m, 1H), 2.70-2.59 (m, 1H), 2.59-2.53 (m, 2H), 2.41 (br dd, J=4.5, 13.2 Hz, 1H), 2.08-1.97 (m, 1H), 1.16 (t, J=7.6 Hz, 3H). MS (ESI) m/z 422.0 [M+H]⁺

Compound 421: To a solution of 4-fluoro-3-nitrophenol (1.00 g, 6.37 mmol, 1.00 eq) in acetone (20.0 mL) were added 2-iodopropane (3.25 g, 19.1 mmol, 1.91 mL, 3.00 eq) and potassium carbonate (2.64 g, 19.1 mmol, 3.00 eq), the mixture was stirred at 70° C. for 12 h. The reaction was filtered to give a filtrate, the filtrate was concentrated to give a residue. The residue was purified by flash silica gel chromatography (ISCO®; 80 g SepaFlash® Silica Flash Column, Eluent of 5-10% Petroleum ether/Ethyl acetate @ 100 mL/min) to afford 1-fluoro-4-isopropoxy-2-nitrobenzene (1.00 g, 5.02 mmol, 78% yield) as yellow oil. ¹H NMR (400 MHz, DMSO-d₆) δ=7.59 (dd, J=3.2, 6.0 Hz, 1H), 7.50 (dd, J=9.2, 10.8 Hz, 1H), 7.36 (td, J=3.6, 9.2 Hz, 1H), 4.70 (spt, J=6.0 Hz, 1H), 1.28 (s, 3H), 1.27 (s, 3H).

To a solution of 1-fluoro-4-isopropoxy-2-nitrobenzene (300 mg, 1.51 mmol, 1.00 eq) in ethyl acetate (6.00 mL) was added wet palladium on carbon (50.0 mg, 10% purity), the mixture was stirred at 25° C. for 12 h. The reaction was filtered to give a filtrate, the filtrate was concentrated to afford 2-fluoro-5-isopropoxyaniline (180 mg, 1.06 mmol, 70% yield) as yellow oil. MS (ESI) m/z 170.1 [M+H]⁺

To a solution of 2-fluoro-5-isopropoxyaniline (180 mg, 1.06 mmol, 1.00 eq) in acetonitrile (2.00 mL) were added phenyl carbonochloridate (174 mg, 1.12 mmol, 139 uL, 1.05 eq) and pyridine (168 mg, 2.13 mmol, 171 uL, 2.00 eq), the mixture was stirred at 25° C. for 2 h. The reaction was concentrated to give a residue. The residue was purified by reversed-phase HPLC (column: Shim-pack C18 150*25*10 um; mobile phase: [water (0.1% formic acid)-acetonitrile]) to afford phenyl (2-fluoro-5-isopropoxyphenyl)carbamate (200 mg, 691 umol, 64% yield) as yellow oil. MS (ESI) m/z 290.1 [M+H]⁺

To a solution of 3-(6-(hydroxymethyl)-1-oxoisoindolin-2-yl)piperidine-2,6-dione 1 (80.0 mg, 291 umol, 1.00 eq) in dimethylformamide (1.00 mL) were added phenyl (2-fluoro-5-isopropoxyphenyl)carbamate (92.8 mg, 320 umol, 1.10 eq) and sodium hydride (23.3 mg, 583 umol, 4.86 uL, 60% purity, 2.00 eq), the mixture was stirred at 25° C. for 1 h. The pH of the mixture was adjusted to 7 with formic acid (0.500 mL), then the mixture was diluted with dimethyl formamide (1.00 mL). The reaction was purified by Prep-HPLC (column: Phenomenex luna C18 150*25 mm*10 um; mobile phase: [water (0.225% formic acid)-acetonitrile]; B %: 33%-63%, 10 min) to afford (2-(2,6-dioxopiperidin-3-yl)-3-oxoisoindolin-5-yl)methyl (2-fluoro-5-isopropoxyphenyl)carbamate 421 (48.3 mg, 101 umol, 34% yield, 99% purity) as a white solid. ¹H NMR (400 MHz, DMSO-d₆) δ=10.99 (br s, 1H), 9.52 (br s, 1H), 7.81 (s, 1H), 7.70-7.60 (m, 2H), 7.26 (br d, J=3.2 Hz, 1H), 7.10 (dd, J=9.2, 10.2 Hz, 1H), 6.64 (td, J=3.6, 8.8 Hz, 1H), 5.27 (s, 2H), 5.13 (dd, J=5.2, 13.2 Hz, 1H), 4.52-4.43 (m, 2H), 4.38-4.28 (m, 1H), 2.98-2.86 (m, 1H), 2.60 (br dd, J=2.4, 15.4 Hz, 1H), 2.40 (dq, J=4.6, 13.2 Hz, 1H), 2.05-1.97 (m, 1H), 1.23 (d, J=6.0 Hz, 6H). MS (ESI) m/z 470.0 [M+H]⁺

Compound 422: To a mixture of 4-fluoro-3-nitrophenol (1.00 g, 6.37 mmol, 1.00 eq) and magnesium perchlorate (852 mg, 3.82 mmol, 0.600 eq) in dichloromethane (10.0 mL) was added the solution of di-tert-butyldicarbonate (6.25 g, 28.6 mmol, 6.58 mL, 4.50 eq) in dichloromethane (10.0 mL) dropwise, the mixture was stirred at 40° C. for 12 h. The reaction mixture was partitioned between dichloromethane (20 mL) and water (30 mL). The organic phase was separated, dried over sodium sulfate, filtered and to give a filtrate, the filtrate was concentrated under reduced pressure to give a residue. The residue was purified by flash silica gel chromatography (ISCO®; 80 g SepaFlash® Silica Flash Column, Eluent of 1-3% Petroleum ether/Ethyl acetate @ 100 mL/min) to afford 4-(tert-butoxy)-1-fluoro-2-nitrobenzene (1.00 g, 4.69 mmol, 73% yield) as yellow oil. ¹H NMR (400 MHz, DMSO-d₆) δ=7.66 (dd, J=3.0, 6.4 Hz, 1H), 7.54-7.48 (m, 1H), 7.46-7.41 (m, 1H), 1.32 (s, 9H).

To a solution of 4-(tert-butoxy)-1-fluoro-2-nitrobenzene (321 mg, 1.51 mmol, 1.00 eq) in ethyl acetate (6.00 mL) was added wet palladium on carbon (50.0 mg, 10% purity), the mixture was stirred at 25° C. for 12 h. The reaction was filtered to give a filtrate, the filtrate was concentrated to afford 5-(tert-butoxy)-2-fluoroaniline (220 mg, 1.20 mmol, 79% yield) as yellow oil. MS (ESI) m/z 184.1 [M+H]⁺

To a solution of 5-(tert-butoxy)-2-fluoroaniline (210 mg, 1.15 mmol, 1.00 eq) in acetonitrile (2.00 mL) were added phenyl carbonochloridate (188 mg, 1.20 mmol, 150 uL, 1.05 eq) and pyridine (181 mg, 2.29 mmol, 185 uL, 2.00 eq), the mixture was stirred at 25° C. for 2 h. The reaction was concentrated to give a residue. The residue was purified by reversed-phase HPLC (column: Shim-pack C18 150*25*10 um; mobile phase: [water (0.1% formic acid)-acetonitrile]) to afford phenyl (5-(tert-butoxy)-2-fluorophenyl)carbamate (220 mg, 725.29 umol, 63.28% yield) as a yellow solid. MS (ESI) m/z 248.1 [M−55]⁺

To a solution of 3-(6-(hydroxymethyl)-1-oxoisoindolin-2-yl)piperidine-2,6-dione 1 (80.0 mg, 291 umol, 1.00 eq) in dimethylformamide (1.00 mL) were added phenyl (5-(tert-butoxy)-2-fluorophenyl)carbamate (97.3 mg, 320 umol, 1.10 eq) and sodium hydride (23.3 mg, 583 umol, 60% purity, 2.00 eq), the mixture was stirred at 25° C. for 1 h. The pH of the mixture was adjusted to 7 with formic acid (0.500 mL), then the mixture was diluted with dimethyl formamide (1.00 mL). The reaction was purified by Prep-HPLC (column: Phenomenex luna C18 150*25 mm*10 um; mobile phase: [water (0.225% FA)-ACN]; B %: 34%-64%, 10 min) to afford (2-(2,6-dioxopiperidin-3-yl)-3-oxoisoindolin-5-yl)methyl (5-(tert-butoxy)-2-fluorophenyl)carbamate 422 (48.2 mg, 98.8 umol, 33% yield, 99% purity) as a white solid. ¹H NMR (400 MHz, DMSO-d₆) δ=10.99 (br s, 1H), 9.53 (br s, 1H), 7.81 (s, 1H), 7.70-7.61 (m, 2H), 7.34 (br d, J=4.2 Hz, 1H), 7.11 (dd, J=9.0, 10.6 Hz, 1H), 6.70 (td, J=3.6, 8.8 Hz, 1H), 5.27 (s, 2H), 5.13 (dd, J=5.2, 13.2 Hz, 1H), 4.51-4.44 (m, 1H), 4.38-4.31 (m, 1H), 2.98-2.86 (m, 1H), 2.64-2.56 (m, 1H), 2.40 (dq, J=4.6, 13.2 Hz, 1H), 2.07-1.96 (m, 1H), 1.26 (s, 9H). MS (ESI) m/z 427.9 [M−55]⁺

Compound 423: To a solution of 4-fluoro-3-nitrophenol (500 mg, 3.18 mmol, 1.00 eq) and potassium carbonate (879 mg, 6.37 mmol, 2.00 eq) in dimethylformamide (5.00 mL) was added iodoethane (595 mg, 3.82 mmol, 305 uL, 1.20 eq), the mixture was stirred at 50° C. for 5 h. The reaction mixture was poured into water (100 mL) and stirred for 10 min. The aqueous phase was extracted with ethyl acetate (3×50.0 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=1/0 to 50/1) to afford 4-ethoxy-1-fluoro-2-nitrobenzene (500 mg, 2.70 mmol, 84% yield) as colorless oil. ¹H NMR (400 MHz, CDCl₃) δ=7.51-7.41 (m, 1H), 7.17-7.03 (m, 2H), 3.99 (q, J=7.0 Hz, 2H), 1.37 (dt, J=0.9, 7.0 Hz, 3H).

To a solution of 4-ethoxy-1-fluoro-2-nitrobenzene (500 mg, 2.70 mmol, 1.00 eq) in methanol (10.0 mL) was added palladium/carbon (50.0 mg, 2.70 mmol, 10% purity), the mixture was stirred at 25° C. for 1 h under hydrogen. The reaction mixture was filtered to give a filtrate, the filtrate was concentrated to give 5-ethoxy-2-fluoro-aniline (400 mg, 2.58 mmol, 95% yield) as yellow oil. MS (ESI) m/z 156.2 [M+H]⁺

To a solution of 5-ethoxy-2-fluoro-aniline (200 mg, 1.29 mmol, 1.00 eq) and pyridine (509 mg, 6.44 mmol, 520 uL, 5.00 eq) in acetonitrile (4.00 mL) was added phenyl carbonochloridate (242 mg, 1.55 mmol, 193 uL, 1.20 eq) at 0° C., the mixture was stirred at 25° C. for 1 h. The reaction mixture was concentrated to give a residue. The residue was purified by reversed-phase HPLC (column: spherical C18, 20-45 um, 40 Å, SW 40, mobile phase: [water (0.1% Formic Acid)-acetonitrile]) to afford phenyl (5-ethoxy-2-fluorophenyl)carbamate (300 mg, 1.09 mmol, 84% yield) as yellow oil. MS (ESI) m/z 276.0[M+H]⁺

To a solution of phenyl (5-ethoxy-2-fluorophenyl)carbamate (120 mg, 437 umol, 1.20 eq) and 3-(6-(hydroxymethyl)-1-oxoisoindolin-2-yl)piperidine-2,6-dione 1 (100 mg, 364 umol, 1.00 eq) in dimethylformamide (1.00 mL) was added sodium hydride (28.0 mg, 700 umol, 60% purity, 1.92 eq) at 0° C., the mixture was stirred at 25° C. for 1 h. The reaction mixture was quenched with formic acid (0.100 mL) and filtered to give a filtrate. The filtrate was purified by Prep-HPLC (column: Phenomenex Synergi C18 150*25 mm*10 um; mobile phase: [water (0.225% formic acid)-acetonitrile]; B %: 30%-60%, 10 min) to afford a crude product. The crude product was purified by Prep-HPLC (column: YMC Triart 30*150 mm*7 um; mobile phase: [water (0.05% hydrochloric acid)-acetonitrile]; B %: 43%-63%, 9 min) to afford (2-(2,6-dioxopiperidin-3-yl)-3-oxoisoindolin-5-yl)methyl (5-ethoxy-2-fluorophenyl)carbamate 423 (33.82 mg, 74.2 umol, 20% yield) as an off white solid. ¹H NMR (400 MHz, DMSO-d₆) δ=11.00 (s, 1H), 9.53 (br s, 1H), 7.82 (s, 1H), 7.74-7.58 (m, 2H), 7.38-7.21 (m, 1H), 7.12 (dd, J=9.0, 10.4 Hz, 1H), 6.65 (td, J=3.4, 9.0 Hz, 1H), 5.28 (s, 2H), 5.14 (dd, J=5.1, 13.2 Hz, 1H), 4.56-4.43 (m, 1H), 4.40-4.30 (m, 1H), 3.96 (q, J=7.0 Hz, 2H), 2.98-2.86 (m, 1H), 2.66-2.57 (m, 1H), 2.47-2.35 (m, 1H), 2.07-1.96 (m, 1H), 1.30 (t, J=7.0 Hz, 3H). MS (ESI) m/z 456.0 [M+H]⁺

Compound 424: To a solution of 3-(tert-butoxycarbonyl)bicyclo[1.1.1]pentane-1-carboxylic acid (100 mg, 471 umol, 1.00 eq) in dioxane (2.00 mL) was added diphenylphosphoryl azide (259 mg, 942 umol, 204 uL, 2.00 eq) and triethylamine (143 mg, 1.41 mmol, 197 uL, 3.00 eq). The mixture was stirred at 25° C. for 0.5 h under nitrogen. Then the 3-(6-(hydroxymethyl)-1-oxoisoindolin-2-yl)piperidine-2,6-dione I (129 mg, 471 umol, 1.00 eq) was added into the mixture and it was stirred at 90° C. for 11.5 h. The mixture was concentrated under reduced pressure to give a residue. The residue was quenched by water (30 mL) and then extracted with ethyl acetate (3×30 mL). The combined organic phases were washed with brine (2×20 mL), dried over anhydrous sodium sulfate, filtered and concentrated in vacuum to give a crude product. The crude product was purified by prep-HPLC (column: Phenomenex Synergi C18 150*25 mm*10 um; mobile phase: [water (0.225% formic acid)-acetonitrile]; B %: 30%-60%, 10 min) and lyophilized to afford tert-butyl 3-((((2-(2,6-dioxopiperidin-3-yl)-3-oxoisoindolin-5-yl)methoxy)carbonyl)amino)bicyclo[1.1.1]pentane-1-carboxylate 424 (34.33 mg, 63.5 umol, 13% yield, 98% purity, formate) as a white solid. ¹H NMR (400 MHz, DMSO-d₆) δ=11.22-10.78 (m, 1H), 8.47 (s, 1H), 8.19-8.06 (m, 1H), 7.69 (s, 1H), 7.60 (s, 2H), 5.17-5.08 (m, 3H), 4.49-4.43 (m, 1H), 4.38-4.28 (m, 1H), 2.96-2.86 (m, 1H), 2.64-2.57 (m, 1H), 2.46-2.37 (m, 1H), 2.11 (s, 6H), 2.04-1.98 (m, 1H), 1.38 (s, 9H). MS (ESI) m/z 428.2 [M−55]⁺

Compound 425: To a solution of dimethyl cubane-1,4-dicarboxylate (200 mg, 908 umol, 1.00 eq) in tetrahydrofuran (6.00 mL) was added sodium hydroxide (2 M in methanol, 499 uL, 1.10 eq). The mixture was stirred at 25° C. for 12 h. The mixture was concentrated dryness to give a residue. The residue was dissolved in water (10.0 mL) and extracted with dichloromethane (10.0 mL). The pH of aqueous phase was adjust to 3-4, and then extracted with ethyl acetate (10.0 mL). The organic phase was concentrated to dryness to give 4-methoxycarbonylcubane-1-carboxylic acid (168 mg, 815 umol, 90% yield) as a white solid. ¹H NMR (400 MHz, CDCl3) δ=4.29 (s, 6H), 3.74 (s, 3H).

To a solution of 4-methoxycarbonylcubane-1-carboxylic acid (100 mg, 485 umol, 1.00 eq) in benzene (20.0 mL) was added lead acetate (280 mg, 630 umol, 1.30 eq) at 25° C. The mixture was stirred at 80° C. for 14 h under irradiation with a mercury lamp. The mixture was filtered and the filtrate was concentrated to dryness to give a residue. The residue was purified by Prep-TLC (Petroleum ether/Ethyl acetate=10/1) to give methyl 4-phenylcubane-1-carboxylate (40.0 mg, 168 umol, 35% yield) as a yellow solid. ¹H NMR (400 MHz, CDCl₃) δ=7.32-7.26 (m, 2H), 7.16-7.11 (m, 3H), 4.20-4.13 (m, 3H), 4.12-4.04 (m, 3H), 3.67 (s, 3H).

To a solution of methyl 4-phenylcubane-1-carboxylate (36.0 mg, 151 umol, 1.00 eq) in tetrahydrofuran (1.00 mL) and methanol (1.00 mL) was added hydrated lithium hydroxide (12.7 mg, 302 umol, 2.00 eq). The mixture was stirred at 25° C. for 12 h. 1 N hydrochloric acid was added to the mixture to pH=3-4, then water (5.00 mL) was added and extracted with ethyl acetate (15.0 mL). The organic phase was washed with brine (5.00 mL), dried over sodium sulfate, filtered and concentrated in vacuum to give 4-phenylcubane-1-carboxylic acid (27.0 mg, 120 umol, 80% yield) as a yellow solid which was used to next step without further purification.

To a mixture of 4-phenylcubane-1-carboxylic acid (27.0 mg, 120 umol, 1.00 eq) and diphenyl phosphoryl azide (53.0 mg, 193 umol, 1.60 eq) in dioxane (2.00 mL) was added triethylamine (24.4 mg, 241 umol, 2.00 eq). The mixture was stirred at 25° C. for 1 h and 3-(6-(hydroxymethyl)-1-oxoisoindolin-2-yl) piperidine-2,6-dione (36.3 mg, 132 umol, 1.10 eq) was added and then stirred at 100° C. for 2 h. The mixture was purified by Prep-HPLC (column: Phenomenex luna C18 150*25 mm*10 um; mobile phase: [water (0.225% FA)-ACN]; B %: 39%-69%, 10 min) to give (2-(2,6-dioxopiperidin-3-yl)-3-oxoisoindolin-5-yl)methyl (4-phenylcuban-1-yl)carbamate 425 (4.44 mg, 8.96 umol, 7.4% yield) as a white solid. ¹H NMR (400 MHz, DMSO-d₆) δ=10.84 (br s, 1H), 8.26 (br s, 1H), 7.74 (s, 1H), 7.63 (s, 2H), 7.39-7.31 (m, 2H), 7.26-7.15 (m, 3H), 5.23-5.08 (m, 3H), 4.52-4.43 (m, 1H), 4.39-4.30 (m, 1H), 3.99 (br d, J=17.8 Hz, 6H), 2.98-2.84 (m, 1H), 2.61 (br d, J=17.4 Hz, 1H), 2.45-2.30 (m, 1H), 2.05-1.96 (m, 1H). MS (ESI) m/z 496.1 [M+1]⁺

Compound 426: To a solution of 4-fluoro-3-nitrophenol (1.00 g, 6.37 mmol, 1.00 eq) in acetone (10.0 mL) was added potassium carbonate (1.76 g, 12.7 mmol, 2.00 eq) and 1-iodopropane (1.30 g, 7.64 mmol, 746 uL, 1.20 eq). The mixture was stirred at 60° C. for 4 h. The reaction mixture was concentrated under reduced pressure to afford a residue. The residue was diluted with water (80.0 mL) and extracted with ethyl acetate (3×30.0 mL). The combined organic layers were washed with brine (30.0 mL), dried over anhydrous sodium sulfate, filtered and concentrated under reduced pressure to give a residue. The residue was purified by column chromatography (SiO₂, petroleum ether/ethyl acetate=1/0 to 10/1) to afford 1-fluoro-2-nitro-4-propoxybenzene (567 mg, 2.85 mmol, 45% yield) as white oil. ¹H NMR (400 MHz, CDCl₃) δ=7.53 (dd, J=2.8, 5.6 Hz, 1H), 7.25-7.12 (m, 2H), 3.97 (t, J=6.4 Hz, 2H), 1.85 (sxt, J=7.2 Hz, 2H), 1.07 (t, J=7.2 Hz, 3H).

To a solution of 1-fluoro-2-nitro-4-propoxybenzene (560 mg, 2.81 mmol, 1.00 eq) in methanol (7.00 mL) was added palladium on carbon (600 mg, 10% purity). The mixture was stirred at 25° C. for 2 h under hydrogen atmosphere (15 Psi). The mixture was filtered, and the filtrate was concentrated under reduced pressure to afford 2-fluoro-5-propoxyaniline (375 mg, 2.22 mmol, 79% yield) as orange oil. ¹H NMR (400 MHz, DMSO-d₆) δ=6.84 (dd, J=8.8, 11.2 Hz, 1H), 6.32 (dd, J=2.9, 7.6 Hz, 1H), 6.03 (td, J=3.2, 8.8 Hz, 1H), 5.07 (s, 2H), 3.79 (t, J=6.4 Hz, 2H), 1.68 (sxt, J=7.2 Hz, 2H), 0.95 (t, J=7.6 Hz, 3H).

To a solution of 2-fluoro-5-propoxy-aniline (200 mg, 1.18 mmol, 1.00 eq) in acetonitrile (2.00 mL) was added phenyl carbonochloridate (194 mg, 1.24 mmol, 155 uL, 1.05 eq) and pyridine (281 mg, 3.55 mmol, 286 uL, 3.00 eq). The mixture was stirred at 25° C. for 2 h. The reaction mixture was concentrated under reduced pressure to afford a residue. The residue was diluted with water (40.0 mL) and extracted with ethyl acetate (3×10.0 mL). The combined organic layers were washed with brine (15.0 mL), dried over anhydrous sodium sulfate, filtered and concentrated under reduced pressure to give a residue. The residue was purified by column chromatography (SiO2, petroleum ether/ethyl acetate=1/0 to 5/1) to afford phenyl (2-fluoro-5-propoxyphenyl)carbamate (258 mg, 892 umol, 75% yield) as colorless oil. ¹H NMR (400 MHz, DMSO-d₆) δ=9.95 (br s, 1H), 7.50-7.37 (m, 2H), 7.36-7.09 (m, 5H), 6.78-6.66 (m, 1H), 3.88 (t, J=6.4 Hz, 2H), 1.71 (t, J=7.2 Hz, 2H), 0.96 (t, J=7.2 Hz, 3H).

To a solution of phenyl (2-fluoro-5-propoxyphenyl)carbamate (92.8 mg, 321 umol, 1.10 eq) in dimethylformamide (500 uL) was added 3-(6-(hydroxymethyl)-1-oxoisoindolin-2-yl)piperidine-2,6-dione 1 (80.0 mg, 292 umol, 1.00 eq) and sodium hydride (23.3 mg, 583 umol, 60% purity, 2.00 eq). The mixture was stirred at 25° C. for 1 h. The pH was adjusted to around 7 by progressively adding formic acid, then the mixture was diluted with dimethyl formamide (1.00 mL). The residue was purified by prep-HPLC (column: Phenomenex luna C18 150*25 mm*10 um; mobile phase: [water (formic acid)-acetonitrile]; B %: 33%-63%, 10 min) and Prep-NPLC (column: Welch Ultimate XB-SiOH 250*50*10 um; mobile phase: [Hexane-ethyl alcohol (0.1% formic acid)]; B %: 1%-35%, 15 min) to afford (2-(2,6-dioxopiperidin-3-yl)-3-oxoisoindolin-5-yl)methyl (2-fluoro-5-propoxyphenyl)carbamate 426 (41.49 mg, 87.5 umol, 30% yield, 99% purity) as a white solid. ¹H NMR (400 MHz, DMSO-d₆) δ=11.00 (br s, 1H), 9.55 (br s, 1H), 7.81 (s, 1H), 7.72-7.59 (m, 2H), 7.35-7.23 (m, 1H), 7.16-7.07 (m, 1H), 6.65 (td, J=3.2, 9.2 Hz, 1H), 5.27 (s, 2H), 5.13 (dd, J=5.1, 13.2 Hz, 1H), 4.53-4.42 (m, 1H), 4.39-4.29 (m, 1H), 3.85 (t, J=6.8 Hz, 2H), 2.99-2.85 (m, 1H), 2.60 (br dd, J=2.0, 15.6 Hz, 1H), 2.47-2.31 (m, 1H), 2.07-1.95 (m, 1H), 1.69 (sxt, J=7.2 Hz, 2H), 0.95 (t, J=7.2 Hz, 3H). MS (ESI) m/z 470.3 [M+H]⁺

Compound 427: To a mixture of 2-chloro-5-fluoropyridin-4-ol (1.00 g, 6.78 mmol, 1.00 eq) and potassium carbonate (2.00 g, 14.5 mmol, 2.13 eq) in N,N-dimethyl formamide (10.0 mL) was added sodium 2-chloro-2,2-difluoroacetate (2.01 g, 13.12 mmol, 1.94 eq). After stirring at 100° C. for 2 h, the mixture was poured into water (50 mL) and extracted with ethyl acetate (2×50 mL). The organic layer was washed with brine (30 mL), dried over anhydrous sodium sulfate, filtered and concentrated to give the crude product. The crude product was purified by flash silica gel chromatography (ISCO®; 20 g SepaFlash® Silica Flash Column, 0˜10% ethyl acetate/petroleum ether=0˜10%, 30 mL/min) to afford 2-chloro-4-(difluoromethoxy)-5-fluoro-pyridine (1.20 g, 6.07 mmol, 90% yield) as colorless oil. ¹H NMR (400 MHz, CDCl₃) δ=8.31 (d, J=1.6 Hz, 1H), 7.24 (d, J=5.6 Hz, 1H), 6.71 (t, J=71.2 Hz, 1H).

A mixture of sodium 2-chloro-2,2-difluoroacetate (1.00 g, 5.06 mmol, 1.00 eq), tert-butyl carbamate (889 mg, 7.59 mmol, 1.50 eq), cesium carbonate (4.95 g, 15.2 mmol, 3.00 eq), tris(dibenzylideneacetone)dipalladium (0) (463 mg, 506 umol, 0.100 eq) and 4,5-bis(diphenylphosphino)-9,9-dimethylxanthene (585 mg, 1.01 mmol, 0.200 eq) in dioxane (20.0 mL) was degassed under vacuum and purged with nitrogen for 3 times. After stirring at 110° C. for 16 h under nitrogen atmosphere, the reaction mixture was poured into saturated ammonium chloride aqueous solution (50 mL) and extracted with ethyl acetate (2×50 mL). The combined organic layer was washed with brine (30 mL), dried over anhydrous sodium sulfate, filtered and concentrated under vacuum to give a residue. The residue was purified by flash silica gel chromatography (ISCO®; 20 g SepaFlashOSilica Flash Column, ethyl acetate/petroleum ether=0˜20%, 50.0 mL/min) to afford tert-butyl N-(4-(difluoromethoxy)-5-fluoro-2-pyridyl)carbamate (500 mg, 1.64 mmol, 32% yield, 91% purity) as a yellow solid. ¹H NMR (400 MHz, CDCl₃) δ=8.18 (s, 1H), 8.11 (s, 1H), 7.94 (d, J=4.4 Hz, 1H), 6.76 (t, J=72.4 Hz, 1H), 1.54 (s, 9H). MS (ESI) m/z 222.9 [M+H]⁺

A solution of tert-butyl (4-(difluoromethoxy)-5-fluoropyridin-2-yl)carbamate (500 mg, 1.64 mmol, 91% purity, 1.00 eq) in hydrochloric acid/dioxane (4 moL/L, 10.0 mL) was stirred at 25° C. for 16 h. The mixture was concentrated under vacuum. The residue was triturated with ethyl acetate (10 mL) and filtered. The filter cake was basified with saturated sodium bicarbonate solution to pH=9. The resulting mixture was extracted with dichloromethane/methanol (10/1, 3×20 mL). The combined organic layers were washed with brine (2×15 mL), dried over anhydrous sodium sulfate, filtered and concentrated under reduced pressure to afford 4-(difluoromethoxy)-5-fluoro-pyridin-2-amine (300 mg, 1.60 mmol, 98% yield, 95% purity) as a white solid. MS (ESI) m/z 179.1 [M+H]⁺

To a solution of 4-(difluoromethoxy)-5-fluoro-pyridin-2-amine (270 mg, 1.44 mmol, 95% purity, 1.00 eq) and pyridine (342 mg, 4.32 mmol, 349 uL, 3.00 eq) in acetonitrile (3.00 mL) was added phenyl carbonochloridate (237 mg, 1.51 mmol, 189 uL, 1.05 eq) at 0° C. After stirring at 25° C. for 2 h, the mixture was poured into water (20 mL) and extracted with ethyl acetate (2×30 mL). The combined organic phase were washed with brine (2×20 mL), dried over anhydrous sodium sulfate, filtered and concentrated under vacuum to give a residue. The residue was purified by flash silica gel chromatography (20 g, ethyl acetate/petroleum ether=0/1-1/4) to afford phenyl (4-(difluoromethoxy)-5-fluoropyridin-2-yl)carbamate (350 mg, 1.16 mmol, 81% yield, 99% purity) as a yellow solid. ¹H NMR (400 MHz, DMSO-d₆) δ=11.07 (s, 1H), 8.47 (d, J=2.0 Hz, 1H), 7.83 (d, J=6.0 Hz, 1H), 7.48-7.41 (m, 3H), 7.31-7.26 (m, 1H), 7.25-7.20 (m, 2H). MS (ESI) m/z 299.0 [M+H]⁺

To a solution of 3-(6-(hydroxymethyl)-1-oxoisoindolin-2-yl)piperidine-2,6-dione 1 (80.0 mg, 292 umol, 1.00 eq) in N,N-dimethyl formamide (2.00 mL) was added sodium hydride (17.5 mg, 437 umol, 60% purity, 1.50 eq) at 0° C. under nitrogen atmosphere. The mixture was stirred at 0° C. for 15 min. Then phenyl (4-(difluoromethoxy)-5-fluoropyridin-2-yl)carbamate (91.5 mg, 292 umol, 95.0% purity, 1.00 eq) was added to the mixture at 0° C. After stirring at 25° C. for 1 h, the reaction mixture was poured into saturated ammonium chloride aqueous solution (10 mL) and extracted with ethyl acetate (2×20 mL). The combined organic layers were washed with brine (20 mL), dried over anhydrous sodium sulfate, filtered and concentrated under vacuum to give a residue. The residue was purified by Prep-HPLC (column: Phenomenex Synergi C18 150*25 mm*10 um; mobile phase: [water (FA)-ACN]; B %: 32%-52%, 10 min) and lyophilized to afford (2-(2,6-dioxopiperidin-3-yl)-3-oxoisoindolin-5-yl)methyl (4-(difluoromethoxy)-5-fluoropyridin-2-yl)carbamate 427 (25.77 mg, 53.3 umol, 18% yield, 99% purity, formate) as an off-white solid. ¹H NMR (400 MHz, DMSO-d₆) δ=10.99 (s, 1H), 10.70 (s, 1H), 8.50 (s, 1H), 8.40 (d, J=2.0 Hz, 1H), 7.85 (d, J=6.4 Hz, 1H), 7.80 (s, 1H), 7.70-7.64 (m, 2H), 7.50 (t, J=72.0 Hz, 1H), 5.32 (s, 2H), 5.16-5.08 (m, 1H), 4.51-4.29 (m, 2H), 2.98-2.85 (m, 1H), 2.65-2.56 (m, 1H), 2.44-2.35 (m, 1H), 2.06-1.96 (m, 1H). MS (ESI) m/z 478.9 [M+H]⁺

Compound 428: To a mixture of 2-bromo-4-fluoro-5-nitro-phenol (5.00 g, 21.1 mmol, 1.00 eq), methylboronic acid (3.80 g, 63.5 mmol, 3.00 eq), 1,1-bis(diphenylphosphino)ferrocene]dichloropalladium(II) (1.55 g, 2.12 mmol, 0.100 eq) and potassium phosphate (13.4 g, 63.5 mmol, 3.00 eq) in dioxane (100 mL) was purged with nitrogen for 3 times. After stirring at 80° C. for 12 h under nitrogen atmosphere, the reaction mixture was poured into saturated ammonium chloride aqueous solution (400 mL) and extracted with ethyl acetate (3×100 mL). The organic phase was concentrated under vacuum to give a residue. The residue was purified by column chromatography (SiO₂, petroleum ether/ethyl acetate=20/1 to 10/1) to afford 4-fluoro-2-methyl-5-nitrophenol (950 mg, 5.00 mmol, 23% yield, 90% purity) as a yellow solid. ¹H NMR (400 MHz, CDCl₃) δ=7.48 (d, J=6.4 Hz, 1H), 7.06 (d, J=11.2 Hz, 1H), 5.59 (s, 1H), 2.33 (s, 3H).

A mixture of 4-fluoro-2-methyl-5-nitro-phenol (400 mg, 2.10 mmol, 90% purity, 1.00 eq), iodoethane (656 mg, 4.21 mmol, 2.00 eq) and sodium carbonate (668 mg, 6.31 mmol, 3.00 eq) in N,N-dimethylformamide (10.0 mL) was stirred at 25° C. for 12 h. The reaction mixture was poured into saturated ammonium chloride aqueous solution (50 mL) and extracted with ethyl acetate (3×20 mL). The organic phase was concentrated under vacuum to give a residue. The residue was purified by column chromatography (petroleum ether/ethyl acetate=30/1 to 10/1) to afford 1-ethoxy-4-fluoro-2-methyl-5-nitro-benzene (420 mg, 1.79 mmol, 85% yield, 85% purity) as colorless oil. ¹H NMR (400 MHz, CDCl₃) δ=7.45 (d, J=6.2 Hz, 1H), 7.06 (d, J=11.2 Hz, 1H), 4.08 (q, J=2.8 Hz, 2H), 2.30 (s, 3H), 1.47 (t, J=2.8 Hz, 3H).

A mixture of 1-ethoxy-4-fluoro-2-methyl-5-nitro-benzene (420 mg, 1.79 mmol, 85% purity, 1.00 eq) and palladium on carbon (42.0 mg, 179 umol, 10% purity, 0.100 eq) in tetrahydrofuran (10.0 mL) was stirred at 25° C. for 4 h under hydrogen atmosphere (15 Psi). The reaction mixture was filtrated and the filtrate was concentrated under reduced pressure to give a residue. The residue was purified by column chromatography (SiO₂, petroleum ether/ethyl acetate=20/1 to 10/1) to afford 5-ethoxy-2-fluoro-4-methyl-aniline (200 mg, 1.12 mmol, 62% yield, 95% purity) as red oil. ¹H NMR (400 MHz, CDCl₃) δ=6.77 (d, J=11.2 Hz, 1H), 6.29 (d, J=7.6 Hz, 1H), 3.94 (q, J=7.2 Hz, 2H), 2.11 (s, 3H), 1.41-1.37 (t, J=7.2 Hz, 3H).

To a mixture of 5-ethoxy-2-fluoro-4-methyl-aniline (200 mg, 1.12 mmol, 95% purity, 1.00 eq) and pyridine (133 mg, 1.68 mmol, 1.50 eq) in tetrahydrofuran (2.00 mL) was added phenyl carbonochloridate (184 mg, 1.18 mmol, 1.05 eq) at 0° C. After addition, the mixture was stirred at 25° C. for 0.5 h. The reaction mixture was poured into saturated ammonium chloride aqueous solution (15 mL) and extracted with ethyl acetate (3×8 mL). The organic phase was concentrated under vacuum to give a residue. The residue was purified by column chromatography (SiO₂, petroleum ether/ethyl acetate=50/1 to 20/1) and concentrated to afford phenyl (5-ethoxy-2-fluoro-4-methylphenyl)carbamate (350 mg, 967 umol, 86% yield, 80% purity) as a white solid. ¹H NMR (400 MHz, DMSO-d₆) δ=9.83 (s, 1H), 7.44-7.38 (m, 2H), 7.26-7.16 (m, 4H), 7.07 (d, J=11.2 Hz, 1H), 3.96 (q, J=7.2 Hz, 2H), 2.11 (s, 3H), 1.31 (t, J=7.2 Hz, 3H).

To a mixture of 3-(6-(hydroxymethyl)-1-oxoisoindolin-2-yl)piperidine-2,6-dione 1 (79.6 mg, 290 umol, 1.05 eq) in N,N-dimethyl formamide (3.00 mL) was added sodium hydride (16.5 mg, 414 umol, 60% purity, 1.50 eq) at 0° C. The mixture was stirred at 0° C. for 0.2 h. Phenyl (5-ethoxy-2-fluoro-4-methylphenyl) carbamate (100 mg, 276 umol, 80% purity, 1.00 eq) was added to the mixture. After stirring at 0° C. for 0.3 h, the reaction mixture was poured into saturated ammonium chloride aqueous solution (20 ml) and extract with ethyl acetate (3×8 mL). The organic phase was concentrated under vacuum to give a residue. The residue was purified by Prep-HPLC (column: Phenomenex Synergi C18 150*25 mm*10 um; mobile phase: [water (FA)-ACN]; B %: 40%-60%, 10 min) and lyophilized to afford (2-(2,6-dioxopiperidin-3-yl)-3-oxoisoindolin-5-yl)methyl(5-ethoxy-2-fluoro-4-methylphenyl)carbamate 428 (91.2 mg, 192 umol, 69% yield, 99% purity) as a white solid. ¹H NMR (400 MHz, DMSO-d₆) δ=10.97 (s, 1H), 9.39 (s, 1H), 7.79 (s, 1H), 7.67-7.61 (m, 2H), 7.25-7.18 (m, 1H), 7.02 (d, J=11.0 Hz, 1H), 5.25 (s, 2H), 5.14-5.09 (m, 1H), 4.46 (d, J=17.6 Hz, 1H), 4.33 (d, J=17.6 Hz, 1H), 3.94 (q, J=6.8 Hz, 2H), 2.98-2.85 (m, 1H), 2.64-2.57 (m, 1H), 2.44-2.35 (m, 1H), 2.09 (s, 3H), 2.04-1.96 (m, 1H), 1.31 (t, J=6.8 Hz, 3H). MS (ESI) m/z 470.2 [M+H]⁺

Compound 429: To a solution of 4-fluoro-2-methyl-5-nitrophenol (400 mg, 2.10 mmol, 90% purity, 1.00 eq) and potassium carbonate (582 mg, 4.21 mmol, 2.00 eq) in N,N-dimethylformamide (5.00 mL) was added iodomethane (896 mg, 6.31 mmol, 393 uL, 3.00 eq). After stirring at 25° C. for 12 h, the mixture was poured into water (20 mL) and extracted with ethyl acetate (3×10 mL). The organic phase was washed with brine (20 mL), dried over anhydrous sodium sulfate, filtered and concentrated under vacuum to give 1-fluoro-4-methoxy-5-methyl-2-nitrobenzene (740 mg, crude) as light-yellow oil. The crude product was used for next step directly without purification. ¹H NMR (400 MHz, DMSO-d₆) δ=7.57 (d, J=6.4 Hz, 1H), 7.45 (d, J=11.2 Hz, 1H), 3.88 (s, 3H), 2.24 (s, 3H).

To a solution of 1-fluoro-4-methoxy-5-methyl-2-nitrobenzene (700 mg, 3.59 mmol, 1.00 eq) in methanol (30 mL) was added palladium on carbon (10% purity, 0.10 g) under nitrogen atmosphere. The suspension was degassed under vacuum and purged with hydrogen for 3 times. The mixture was stirred at 25° C. for 12 h under hydrogen atmosphere (15 Psi). After filtration, the filtrate was concentrated under vacuum to give a residue. The residue was purified by column chromatography (SiO₂, petroleum ether then petroleum ether/ethyl acetate=5/1) to afford 2-fluoro-5-methoxy-4-methylaniline (240 mg, 1.47 mmol, 41% yield, 95% purity) as a red solid. ¹H NMR (400 MHz, DMSO-d₆) δ=6.75 (d, J=12.0 Hz, 1H), 6.37 (d, J=8.0 Hz, 1H), 4.87 (s, 2H), 3.67 (s, 3H), 1.98 (s, 3H)

To a solution of 2-fluoro-5-methoxy-4-methylaniline (200 mg, 1.22 mmol, 95% purity, 1.00 eq) in acetonitrile (3.00 mL) was added pyridine (291 mg, 3.67 mmol, 297 uL, 3.00 eq) at 0° C. After stirring the mixture at 0° C. for 0.2 h, phenyl carbonochloridate (211 mg, 1.35 mmol, 169 uL, 1.10 eq) was added to the mixture. After addition, the mixture was stirred at 25° C. for 1.8 h. The mixture was poured into water (50 mL) and extracted with ethyl acetate (3×10 mL). The organic phase was washed with brine (20 mL), dried over anhydrous sodium sulfate, filtered and concentrated under vacuum to give a residue. The residue was purified by column chromatography (SiO₂, petroleum ether then petroleum ether/ethyl acetate=4/1) to afford phenyl (2-fluoro-5-methoxy-4-methylphenyl)carbamate (240 mg, 828 umol, 68% yield, 95% purity) as white solid. ¹H NMR (400 MHz, DMSO-d₆) δ=9.84 (s, 1H), 7.45-7.37 (m, 2H), 7.23-7.17 (m, 3H), 6.76-6.71 (m, 2H), 3.73 (s, 3H), 2.11 (s, 3H).

To a solution of 3-(6-(hydroxymethyl)-1-oxoisoindolin-2-yl)piperidine-2,6-dione 1 (99.6 mg, 363 umol, 1.00 eq) in N,N-dimethylformamide (3.00 mL) was added sodium hydride (29.1 mg, 727 umol, 60% purity, 2.00 eq) at 0° C. The mixture was stirred at 0° C. for 0.1 h. Then phenyl (2-fluoro-5-methoxy-4-methylphenyl)carbamate (100 mg, 363 umol, 1.00 eq) in N,N-dimethylformamide (0.500 mL) was added dropwise to the mixture at 0° C. After stirring at 25° C. for 0.4 h, the mixture was poured into saturated ammonium chloride aqueous solution (20 mL) and extracted with ethyl acetate (3×10 mL). The organic phase was washed with saturated sodium chloride aqueous solution (20 mL), brine (20 ml), dried over anhydrous sodium sulfate, filtered and concentrated under vacuum to give a residue. The residue was purified by Prep-HPLC (column: Phenomenex Synergi C18 150*25 mm*10 um; mobile phase: [water (FA)-ACN]; B %: 30%-60%, 10 min). to afford (2-(2,6-dioxopiperidin-3-yl)-3-oxoisoindolin-5-yl)methyl (2-fluoro-5-methoxy-4-methylphenyl)carbamate 429 (61.48 mg, 134 umol, 37% yield, 99% purity) as white solid. ¹H NMR (400 MHz, DMSO-d₆) δ=10.98 (s, 1H), 9.41 (s, 1H), 7.80 (s, 1H), 7.70-7.60 (m, 2H), 7.25-7.17 (m, 1H), 7.03 (d, J=11.2 Hz, 1H), 5.26 (s, 2H), 5.17-5.06 (m, 1H), 4.52-4.28 (m, 2H), 3.72 (s, 3H), 2.98-2.85 (m, 1H), 2.69-2.56 (m, 1H), 2.44-2.37 (m, 1H), 2.10 (s, 3H), 2.06-1.96 (m, 1H). MS (ESI) m/z 456.2 [M+H]⁺

Compound 430: To a mixture of 4-fluoro-2-methyl-5-nitro-phenol (250 mg, 1.46 mmol, 1.00 eq) and potassium carbonate (605 mg, 4.38 mmol, 3.00 eq) in N,N-dimethylformamide (10.0 mL) was added 2-iodopropane (745 mg, 4.38 mmol, 3.00 eq). After stirring at 50° C. for 2 h, the reaction mixture was poured into saturated ammonium chloride aqueous solution (40 mL) and extracted with ethyl acetate (3×20 mL). The organic phase was concentrated under vacuum to give a residue. The residue was purified by column chromatography (SiO₂, petroleum ether/ethyl acetate=50/1 to 20/1) to afford 1-fluoro-4-isopropoxy-5-methyl-2-nitro-benzene (150 mg, 633 umol, 43% yield, 90% purity) as colorless oil. ¹H NMR (400 MHz, CDCl₃) δ=7.47 (d, J=6.4 Hz, 1H), 7.06 (d, J=11.2 Hz, 1H), 4.59-4.53 (m, 1H), 2.27 (s, 3H), 1.38 (d, J=6.0 Hz, 6H).

A mixture of 1-fluoro-4-isopropoxy-5-methyl-2-nitro-benzene (120 mg, 506 umol, 90% purity, 1.00 eq) and palladium on carbon (12.0 mg, 50.6 umol, 10% purity, 0.100 eq) in tetrahydrofuran (5.00 mL) was stirred at 25° C. for 5 h under hydrogen atmosphere (15 Psi). The reaction mixture was filtered and the filtrate was concentrated under vacuum to give a residue. The residue was purified by column chromatography (SiO₂, petroleum ether/ethyl acetate=30/1 to 10/1) and concentrated to afford 2-fluoro-5-isopropoxy-4-methylaniline (110 mg, 90% yield, 90% purity) as red oil. ¹H NMR (400 MHz, CDCl₃) δ=6.77 (d, J=11.4 Hz, 1H), 6.33 (d, J=8.0 Hz, 1H), 4.38-4.29 (m, 1H), 3.51 (br s, 2H), 2.09 (s, 3H), 1.30 (d, J=6.0 Hz, 6H).

To a mixture of phenyl (2-fluoro-5-isopropoxy-4-methylphenyl)carbamate (100 mg, 491 umol, 90% purity, 1.00 eq) and pyridine (58.2 mg, 736 umol, 1.50 eq) in tetrahydrofuran (5.00 mL) was added phenyl carbonochloridate (80.7 mg, 515 umol, 1.05 eq) at 0° C. After addition, the mixture was stirred at 25° C. for 0.5 h. The reaction mixture was poured into saturated ammonium chloride aqueous solution (30 mL) and extracted with ethyl acetate (3×15 mL). The organic phase was concentrated under vacuum to give a residue. The residue was purified by column chromatography (SiO₂, petroleum ether/ethyl acetate=50/1 to 20/1) to afford phenyl (2-fluoro-5-isopropoxy-4-methylphenyl)carbamate (140 mg, 85% yield, 90% purity) as a red solid. ¹H NMR (400 MHz, CDCl₃) δ=7.72-7.71 (m, 1H), 7.46-7.38 (m, 2H), 7.27-7.24 (m, 1H), 7.23-7.19 (m, 2H), 7.12 (s, 1H), 6.90 (d, J=11.6 Hz, 1H), 4.50-4.44 (m, 1H), 2.16 (s, 3H), 1.31 (d, J=6.0 Hz, 6H).

To a mixture of 3-(6-(hydroxymethyl)-1-oxoisoindolin-2-yl)piperidine-2,6-dione 1 (87.9 mg, 321 umol, 0.90 eq) in N,N-dimethylformamide (2.00 mL) was added sodium hydride (28.5 mg, 712 umol, 60% purity, 2.00 eq) at 0° C. After stirring at 0° C. for 0.2 h, phenyl (2-fluoro-5-isopropoxy-4-methylphenyl)carbamate (120 mg, 356 umol, 90% purity, 1.00 eq) in N,N-dimethylformamide (0.50 mL) was added to the mixture. After stirring at 25° C. for 0.3 h, the reaction mixture was poured into saturated ammonium chloride aqueous solution (20 mL), extracted with ethyl acetate (3×20 mL), washed with brine (20 mL), dried over anhydrous sodium sulfate, filtered and concentrated. The organic phase was concentrated under vacuum to give a residue. The residue was purified by Prep-HPLC (column: Phenminomenex Synergi C18 150*25 mm*10 um; mobile phase: [water (FA)-ACN]; B %: 43%-63%, 10 min) and then lyophilized to afford (2-(2,6-dioxopiperidin-3-yl)-3-oxoisoindolin-5-yl)methyl (2-fluoro-5-isopropoxy-4-methylphenyl)carbamate 430 (92.22 mg, 189 umol, 53% yield, 99% purity) as a white solid. ¹H NMR (400 MHz, DMSO-d₆) δ=11.99 (s, 1H), 9.38 (s, 1H), 7.80 (s, 1H), 7.71-7.59 (m, 2H), 7.28-7.17 (m, 1H), 7.01 (d, J=11.2 Hz, 1H), 5.26 (s, 2H), 5.17-5.08 (m, 1H), 4.53-4.29 (m, 3H), 2.98-2.86 (m, 1H), 2.64-2.56 (m, 1H), 2.44-2.37 (m, 1H), 2.11-2.05 (m, 3H), 2.05-1.96 (m, 1H), 1.24 (d, J=6.0 Hz, 6H). MS (ESI) m/z 484.1 [M+H]⁺

Compound 431: To a solution of 4-fluoro-2-methyl-5-nitrophenol (400 mg, 1.99 mmol, 1.00 eq) in N,N-dimethylformamide (10.0 mL) were added 1-iodopropane (1.01 g, 5.96 mmol, 582 uL, 3.00 eq) and potassium carbonate (823 mg, 5.96 mmol, 3.00 eq). After addition, the mixture was stirred at 25° C. for 1 h. The mixture was poured into water (20 mL) and extracted with ethyl acetate (3×10 mL). The organic phase was washed with brine (20 ml), dried over anhydrous sodium sulfate, filtered and concentrated under vacuum to give 1-fluoro-5-methyl-2-nitro-4-propoxybenze (700 mg, crude) as red oil. The crude product was used for next step directly without purification. ¹H NMR (400 MHz, DMSO-d₆) δ=7.56 (d, J=6.4 Hz, 1H), 7.46 (d, J=12.4 Hz, 1H), 4.03 (t, J=6.4 Hz, 2H), 2.25 (s, 3H), 1.82-1.71 (m, 2H), 1.00 (t, J=7.2 Hz, 3H).

To a solution of 1-fluoro-5-methyl-2-nitro-4-propoxybenzene (700 mg, 3.28 mmol, 1.00 eq) in methanol (10.0 mL) was added palladium on carbon (10% purity, 0.10 g) under nitrogen atmosphere. The suspension was degassed and purged with hydrogen for 3 times. The mixture was stirred at 25° C. for 2 h under hydrogen atmosphere (15 Psi). After filtration, the filtrate was concentrated under vacuum to give a residue. The residue was purified by column chromatography (SiO₂, petroleum ether/ethyl acetate=1/0 to 4/1) to afford 2-fluoro-4-methyl-5-propoxyaniline (240 mg, 1.27 mmol, 39% yield, 97% purity) as a yellow solid. ¹H NMR (400 MHz, DMSO-d₆) δ=6.78 (d, J=11.2 Hz, 1H), 6.34 (d, J=7.6 Hz, 1H), 3.83 (t, J=6.4 Hz, 2H), 3.50-2.63 (m, 2H), 2.12 (s, 3H), 1.86-1.75 (m, 2H), 1.04 (t, J=7.2 Hz, 3H).

To a solution of 2-fluoro-4-methyl-5-propoxy-aniline (140 mg, 764 umol, 1.00 eq) in acetonitrile (3.00 mL) was added pyridine (181 mg, 2.29 mmol, 185 uL, 3.00 eq) at 0° C. After stirring the mixture for 10 min, phenyl carbonochloridate (132 mg, 841 umol, 105 uL, 1.10 eq) was added to the mixture. After addition, the mixture was stirred at 25° C. for 1 h. The mixture was poured into water (20 mL) and extracted with ethyl acetate (3×10 mL). The organic phase was washed with brine (30 mL), dried over anhydrous sodium sulfate, filtered and concentrated under vacuum to give a residue. The residue was purified by column chromatography (SiO₂, petroleum ether then petroleum ether/ethyl acetate=10/1) to afford phenyl (2-fluoro-4-methyl-5-propoxyphenyl)carbamate (100 mg, 320 umol, 42% yield, 97% purity) as a yellow solid. ¹H NMR (400 MHz, DMSO-d₆) δ=9.84 (s, 1H), 7.42 (t, J=8.0 Hz, 2H), 7.28-7.22 (m, 1H), 7.22-7.18 (m, 2H), 7.07 (d, J=11.2 Hz, 1H), 6.78-6.72 (m, 1H), 3.86 (t, J=6.4 Hz, 2H), 2.12 (s, 3H), 1.77-1.67 (m, 2H), 0.98 (t, J=7.2 Hz, 3H).

To a solution of 3-(6-(hydroxymethyl)-1-oxoisoindolin-2-yl)piperidine-2,6-dione 1 (81.4 mg, 297 umol, 1.00 eq) in N,N-dimethylformamide (3.00 mL) was added sodium hydride (23.7 mg, 593 umol, 60% purity, 2.00 eq) at 0° C. After addition, the mixture was stirred at 0° C. for 10 min, then phenyl (2-fluoro-4-methyl-5-propoxyphenyl)carbamate (90 mg, 297 umol, 1.00 eq) in N,N-dimethylformamide (1.00 mL) was added dropwise to the mixture at 0° C. After stirring at 25° C. for 20 min, the mixture was poured into saturated ammonium chloride aqueous solution (20 mL) and extracted with ethyl acetate (3×10 mL). The organic phase was washed with brine (20.0 ml), dried over anhydrous sodium sulfate, filtered and concentrated under vacuum to give a residue. The residue was purified by Prep-HPLC (FA condition; column: Phenomenex Synergi C18 150*25 mm*10 um; mobile phase: [water (FA)-ACN]; B %: 45%-67%, 11 min) to afford (2-(2,6-dioxopiperidin-3-yl)-3-oxoisoindolin-5-yl)methyl (2-fluoro-4-methyl-5-propoxyphenyl)carbamate 431 (62.0 mg, 127 umol, 43% yield, 99% purity) as a white solid. ¹H NMR (400 MHz, DMSO-d₆) δ=10.99 (s, 1H), 9.41 (s, 1H), 7.80 (s, 1H), 7.72-7.56 (m, 2H), 7.29-7.13 (m, 1H), 7.02 (d, J=11.2 Hz, 1H), 5.26 (s, 2H), 5.19-5.09 (m, 1H), 4.52-4.28 (m, 2H), 3.84 (t, J=6.4 Hz, 2H), 2.99-2.83 (m, 1H), 2.64-2.57 (m, 1H), 2.44-2.37 (m, 1H), 2.10 (s, 3H), 2.04-1.94 (m, 1H), 1.78-1.65 (m, 2H), 0.97 (t, J=7.2 Hz, 3H). MS (ESI) m/z 484.0 [M+H]⁺

Compound 432: To a solution of 2,5-dibromo-3-fluoropyridine (5.00 g, 19.6 mmol, 1.00 eq) in dioxane (50.0 mL) was added diphenylmethanimine (3.91 g, 21.6 mmol, 3.62 mL, 1.10 eq), cesium carbonate (19.2 g, 58.9 mmol, 3.00 eq), 4,5-bis(diphenylphosphino)-9,9-dimethylxanthene (2.27 g, 3.92 mmol, 0.200 eq) and tris(dibenzylideneacetone)dipalladium(0) (1.80 g, 1.96 mmol, 0.100 eq) under nitrogen atmosphere. The mixture was stirred at 80° C. for 12 h. The mixture was diluted with water (300 mL) and extracted with ethyl acetate (3×80 mL). The combined organic layer was washed with brine (50 mL), dried over anhydrous sodium sulfate, filtered and concentrated to give a crude product. The crude product was purified by column chromatography on silica gel (petroleum ether/ethyl acetate=1/0 to 10/1) to afford 5-bromo-N-(diphenylmethylene)-3-fluoropyridin-2-amine (4.50 g, 9.25 mmol, 47% yield) as a yellow solid. ¹H NMR (400 MHz, DMSO-d6) δ=8.24 (d, J=1.6 Hz, 1H), 7.99 (dd, J=2.0, 9.6 Hz, 1H), 7.72 (br d, J=7.6 Hz, 2H), 7.66-7.58 (m, 1H), 7.56-7.47 (m, 2H), 7.41-7.32 (m, 3H), 7.13 (br d, J=6.4 Hz, 2H).

To a solution of 5-bromo-N-(diphenylmethylene)-3-fluoropyridin-2-amine (300 mg, 845 umol, 1.00 eq) in dioxane (3.00 mL) was added (R)-2-methylpyrrolidine (154 mg, 1.27 mmol, 1.50 eq, hydrochloric acid), cesium carbonate (1.10 g, 3.38 mmol, 4.00 eq) and (2-dicyclohexylphosphino-2′,b′-dimethoxybiphenyl)[2-(2′-amino-1,1′-biphenyl)]palladium(II)methanesulfonate (65.9 mg, 84.5 umol, 0.100 eq) under nitrogen atmosphere. The mixture was stirred at 110° C. for 12 h. The reaction mixture was diluted with water (30 mL) and extracted with ethyl acetate (3×10 mL). The combined organic layers were washed with brine (10 mL), dried over anhydrous sodium sulfate, filtered and concentrated under reduced pressure to give a residue. The residue was purified by column chromatography on silica gel (petroleum ether/ethyl acetate=1/0 to 3/1) to afford (R)—N-(diphenylmethylene)-3-fluoro-5-(2-methylpyrrolidin-1-yl)pyridin-2-amine (450 mg, 1.25 mmol, 49% yield) as a yellow solid. ¹H NMR (400 MHz, DMSO-d6) δ=7.70-7.63 (m, 2H), 7.58-7.51 (m, 1H), 7.50-7.44 (m, 2H), 7.42 (d, J=1.2 Hz, 1H), 7.36-7.29 (m, 3H), 7.10 (dd, J=2.7, 6.0 Hz, 2H), 6.71 (dd, J=2.2, 13.2 Hz, 1H), 3.81 (brt, J=6.0 Hz, 1H), 3.29 (brt, J=7.6 Hz, 1H), 3.01 (q, J=8.4 Hz, 1H), 2.01-1.84 (m, 3H), 1.61 (br s, 1H), 1.01 (d, J=6.0 Hz, 3H).

To a solution of (R)—N-(diphenylmethylene)-3-fluoro-5-(2-methylpyrrolidin-1-yl)pyridin-2-amine (400 mg, 1.11 mmol, 1.00 eq) in tetrahydrofuran (24.0 mL) was added hydrochloric acid (5 M, 4.00 mL, 18.0 eq). The mixture was stirred at 25° C. for 2 h. The pH of the mixture was adjust to 8 with sodium hydroxide saturated solution. Then the mixture was diluted with water (50 mL) and extracted with ethyl acetate (4×20 mL). The residue was purified by column chromatography on silica gel (Petroleum ether/Ethyl acetate=1/0 to 3/1) to afford (R)-3-fluoro-5-(2-methylpyrrolidin-1-yl)pyridin-2-amine (200 mg, 1.02 mmol, 92% yield) as a white solid. ¹H NMR (400 MHz, DMSO-d6) δ=7.21 (d, J=2.0 Hz, 1H), 6.80 (dd, J=2.4, 13.2 Hz, 1H), 5.14 (s, 2H), 3.77-3.64 (m, 1H), 3.30-3.24 (m, 1H), 2.98 (q, J=8.4 Hz, 1H), 2.00-1.84 (m, 3H), 1.66-1.55 (m, 1H), 1.04 (d, J=6.4 Hz, 3H).

To a solution of (R)-3-fluoro-5-(2-methylpyrrolidin-1-yl)pyridin-2-amine (100 mg, 512 umol, 1.00 eq) in acetonitrile (0.500 mL) was added pyridine (122 mg, 1.54 mmol, 124 uL, 3.00 eq) and phenyl carbonochloridate (84.2 mg, 538 umol, 67.4 uL, 1.05 eq). The mixture was stirred at 25° C. for 2 h. The reaction mixture was concentrated under reduced pressure to afford a residue. The residue was purified by reverse-phase HPLC (column: spherical C18, 20-45 um, 100 Å, SW 120, mobile phase:[water (0.1% formic acid)-acetonitrile]) and the desired eluent was lyophilized to afford (R)-phenyl(3-fluoro-5-(2-methylpyrrolidin-1-yl)pyridin-2-yl)carbamate (100 mg, 311 umol, 61% yield, 98% purity) as a black solid. ¹H NMR (400 MHz, DMSO-d6) δ=9.37 (br s, 1H), 7.54 (d, J=2.0 Hz, 1H), 7.20 (d, J=2.0 Hz, 1H), 7.15 (t, J=8.0 Hz, 3H), 6.93 (dd, J=2.3, 12.8 Hz, 1H), 6.80 (dd, J=2.4, 13.6 Hz, 1H), 3.89 (brt, J=6.0 Hz, 1H), 3.16-3.05 (m, 1H), 2.98 (q, J=8.4 Hz, 1H), 2.01-1.93 (m, 3H), 1.57-1.53 (m, 1H), 1.04 (d, J=6.0 Hz, 3H). MS (ESI) m/z 316.1 [M+H]⁺

To a solution of 3-(6-(hydroxymethyl)-1-oxoisoindolin-2-yl)piperidine-2,6-dione 1 (45.4 mg, 166 umol, 0.950 eq) in dimethyl formamide (300 uL) was added sodium hydride (14.0 mg, 349 umol, 60% purity, 2.00 eq). The mixture was stirred at 0° C. for 0.5 h. Then a solution of (R)-phenyl (3-fluoro-5-(2-methylpyrrolidin-1-yl)pyridin-2-yl)carbamate (55.0 mg, 174 umol, 1.00 eq) in dimethyl formamide (200 uL) was added to the mixture dropwise. The mixture was stirred at 25° C. for 1 h. The pH of the mixture was adjusted to 7 with formic acid, then the mixture was diluted with dimethyl formamide (1.00 mL). The mixture was purified by prep-HPLC (column: Phenomenex luna C18 150*25 mm*10 um; mobile phase: [water (formic acid)-acetonitrile]; B %: 26%-56%, 10 min) and the desired eluent was lyophilized to afford (2-(2,6-dioxopiperidin-3-yl)-3-oxoisoindolin-5-yl)methyl (3-fluoro-5-((R)-2-methylpyrrolidin-1-yl)pyridin-2-yl)carbamate 432 (18.23 mg, 35.3 umol, 20% yield, 96% purity) as a white solid. ¹H NMR (400 MHz, DMSO-d6) δ=11.00 (br s, 1H), 9.33 (s, 1H), 7.75 (s, 1H), 7.63 (s, 2H), 7.56 (d, J=2.0 Hz, 1H), 6.87 (dd, J=2.2, 12.4 Hz, 1H), 5.21 (s, 2H), 5.13 (dd, J=4.9, 13.2 Hz, 1H), 4.51-4.42 (m, 1H), 4.38-4.28 (m, 1H), 3.95-3.86 (m, 1H), 3.40-3.37 (m, 1H), 3.16-3.06 (m, 1H), 2.97-2.87 (m, 1H), 2.62-2.58 (m, 1H), 2.40 (br dd, J=4.6, 13.2 Hz, 1H), 2.05-1.93 (m, 4H), 1.67 (br s, 1H), 1.09 (d, J=6.0 Hz, 3H). MS (ESI) m/z 496.3 [M+H]⁺

Compound 433: To a mixture of 1-bromo-4-fluoro-2-methyl-5-nitro-benzene (4.00 g, 17.1 mmol, 1.00 eq) in N-methyl pyrrolidone (40.0 mL) were added copper (I) iodide (4.88 g, 25.6 mmol, 1.50 eq) and methyl 2,2-difluoro-2-fluorosulfonyl-acetate (18.1 g, 94.3 mmol, 12.0 mL, 5.52 eq). After stirring at 150° C. for 16 h under nitrogen atmosphere, the reaction mixture was poured into saturated ammonium chloride aqueous solution (50 mL) and extracted with ethyl acetate (2×100 mL). The combined organic layers were washed with brine (30 mL), dried over anhydrous sodium sulfate, filtered and concentrated under vacuum to give a residue. The residue was purified by flash silica gel chromatography (ISCO®; 20 g SepaFlash® Silica Flash Column, ethyl acetate/petroleum ether=0-10%, 50.0 mL/min) to afford 1-fluoro-5-methyl-2-nitro-4-(trifluoromethyl)benzene (3.00 g, 13.4 mmol, 78% yield) as yellow oil. ¹H NMR (400 MHz, CDCl₃) δ=8.39 (d, J=7.2 Hz, 1H), 7.27 (d, J=11.2 Hz, 1H), 2.60 (s, 3H).

To a solution of 1-fluoro-5-methyl-2-nitro-4-(trifluoromethyl)benzene (1.00 g, 4.48 mmol, 1.00 eq) in methanol (20.0 mL) was added palladium on carbon (500 mg, 10% purity) under nitrogen atmosphere. The suspension was degassed under vacuum and purged with hydrogen (15 Psi) for 3 times. After stirring at 25° C. for 16 h under hydrogen atmosphere (15 Psi). The reaction mixture was filtered. The filtrate was concentrated to give a residue. The residue was purified by flash silica gel chromatography (ISCO®; 20 g SepaFlash® Silica Flash Column, ethyl acetate/petroleum ether=0-20%, 40 mL/min) to afford 2-fluoro-4-methyl-5-(trifluoromethyl)aniline (140 mg, 725 umol, 16% yield) as yellow oil. ¹H NMR (400 MHz, CDCl₃) δ=7.10 (d, J=8.4 Hz, 1H), 6.95-6.87 (m, 1H), 2.36 (s, 3H).

To a solution of 2-fluoro-4-methyl-5-(trifluoromethyl)aniline (120 mg, 621 umol, 1.00 eq) and pyridine (147 mg, 1.86 mmol, 150 uL, 3.00 eq) in acetonitrile (2.00 mL) was added phenyl carbonochloridate (97.3 mg, 621 umol, 77.8 uL, 1.00 eq) at 0° C. After stirring the mixture at 25° C. for 1 h, the mixture was poured into water (10 mL) and extracted with ethyl acetate (2×20 mL). The combined organic phase was washed with brine (2×10 mL), dried over anhydrous sodium sulfate, filtered and concentrated to give a residue. The residue was purified by flash silica gel chromatography (ethyl acetate/petroleum ether=0/1 to 1/10) to afford phenyl (2-fluoro-4-methyl-5-(trifluoromethyl)phenyl)carbamate (70.0 mg, 185 umol, 30% yield, 83% purity) as yellow oil. ¹H NMR (400 MHz, DMSO-d₆) δ=8.53-8.40 (m, 1H), 7.47-7.39 (m, 2H), 7.28 (s, 1H), 7.24-7.19 (m, 2H), 7.09-7.03 (m, 1H), 2.50-2.43 (m, 3H). MS (ESI) m/z 314.0 [M+H]⁺

To a solution of 3-(6-(hydroxymethyl)-1-oxoisoindolin-2-yl)piperidine-2,6-dione 1 (26.3 mg, 95.8 umol, 1.00 eq) in N,N-dimethyl formamide (1.00 mL) was added sodium hydride (6.00 mg, 150 umol, 60% purity, 1.57 eq) at 0° C. under nitrogen atmosphere. The mixture was stirred at 0° C. for 15 min, and then phenyl (2-fluoro-4-methyl-5-(trifluoromethyl)phenyl)carbamate (30.0 mg, 95.8 umol, 1.00 eq) was added to the mixture at 0° C. After stirring at 25° C. for 1 h, the reaction mixture was poured into saturated ammonium chloride aqueous solution (10 mL) and extracted with ethyl acetate (2×20 mL). The combined organic layers were washed with brine (20 mL), dried over anhydrous sodium sulfate, filtered and concentrated under vacuum to give a residue. The residue was purified by Prep-HPLC (column: Phenomenex Synergi C18 150*25 mm*10 um; mobile phase: [water (FA)-ACN]; B %: 41%-61%, 10 min) and lyophilized to afford (2-(2,6-dioxopiperidin-3-yl)-3-oxoisoindolin-5-yl)methyl(2-fluoro-4-methyl-5-(trifluoromethyl)phenyl)carbamate 433 (23.0 mg, 45.3 umol, 47% yield, 97% purity, formate) as a white solid. ¹H NMR (400 MHz, DMSO-d₆) δ=11.00 (s, 1H), 9.82 (s, 1H), 8.45 (s, 1H), 8.06 (d, J=6.0 Hz, 1H), 7.82 (s, 1H), 7.74-7.67 (m, 1H), 7.66-7.61 (m, 1H), 7.40 (d, J=11.6 Hz, 1H), 5.30 (s, 2H), 5.18-5.09 (m, 1H), 4.52-4.31 (m, 2H), 2.98-2.86 (m, 1H), 2.65-2.57 (m, 1H), 2.44-2.36 (m, 4H), 2.06-1.97 (m, 1H). MS (ESI) m/z 494.2 [M+H]⁺

Compound 434: To a solution of 1-(4-fluoro-3-nitro-phenyl)ethanone (5.00 g, 27.3 mmol, 1.00 eq) in dichloromethane (60.0 mL) was added diethylaminosulfurtrifluoride (22.0 g, 137 mmol, 18.0 mL, 5.00 eq) dropwise at −70° C. The mixture was stirred at 25° C. for 12 h. The reaction mixture was poured into sodium bicarbonate aqueous solution (300 mL). The mixture was extracted with ethyl acetate (3×100 mL), washed with brine, dried over anhydrous sodium sulfate, filtered and concentrated under reduced pressure to give a residue. The residue was purified by column chromatography (SiO₂, petroleum ether/ethyl acetate=20/1 to 8/1) to afford 4-(1,1-difluoroethyl)-1-fluoro-2-nitrobenzene (4.10 g, 20.0 mmol, 73% yield) as colorless oil. ¹H NMR (400 MHz, DMSO-d₆) δ=8.32-8.28 (m, 1H), 8.06-8.02 (m, 1H), 7.77-7.71 (m, 1H), 2.04 (t, J=19.2 Hz, 3H).

To a solution of 4-(1,1-difluoroethyl)-1-fluoro-2-nitrobenzene (1.50 g, 7.31 mmol, 1.00 eq) in methanol (20.0 mL) and water (20.0 mL) were added iron powder (2.04 g, 36.6 mmol, 5.00 eq) and ammonium chloride (3.91 g, 73.1 mmol, 10.0 eq). The mixture was stirred at 80° C. for 12 h. After filtration, the filtrate was concentrated under vacuum to give a residue. The residue was purified by column chromatography (SiO₂, petroleum ether then petroleum ether/ethyl acetate=5/1) to afford 5-(1,1-difluoroethyl)-2-fluoro-aniline (490 mg, 2.52 mmol, 34% yield, 90% purity) as light-yellow oil. ¹H NMR (400 MHz, DMSO-d₆) δ=7.05 (dd, J=8.4, 11.2 Hz, 1H), 6.94 (dd, J=2.4, 8.8 Hz, 1H), 6.70-6.62 (m, 1H), 5.36 (s, 2H), 1.88 (t, J=18.8 Hz, 3H).

To a solution of 5-(1,1-difluoroethyl)-2-fluoro-aniline (200 mg, 1.03 mmol, 90% purity, 1.00 eq) and pyridine (244 mg, 3.08 mmol, 249 uL, 3.00 eq) in acetonitrile (3.00 mL) was added phenyl carbonochloridate (177 mg, 1.13 mmol, 142 uL, 1.10 eq) dropwise at 0° C. over 30 min. The resulting mixture was stirred at 25° C. for 2.5 h. The mixture was poured into water (50.0 mL) and extracted with ethyl acetate (3×10.0 mL). The organic phase was washed by brine (20.0 mL), dried over anhydrous sodium sulfate, filtered and concentrated under reduced pressure to give a residue. The residue was purified by column chromatography (SiO₂, petroleum ether/ethyl acetate=1/0 to 20/1) to afford phenyl (5-(1,1-difluoroethyl)-2-fluorophenyl)carbamate (320 mg, 759 umol, 74% yield, 70% purity) as a light yellow solid. ¹H NMR (400 MHz, DMSO-d₆) δ=10.16 (s, 1H), 7.94 (d, J=6.8 Hz, 1H), 7.44-7.38 (m, 3H), 7.30-7.22 (m, 3H), 7.18-7.13 (m, 1H), 1.95 (t, J=18.8 Hz, 3H).

To a solution of phenyl (5-(1,1-difluoroethyl)-2-fluorophenyl)carbamate (120 mg, 285 umol, 70% purity, 1.00 eq) and 3-(6-(hydroxymethyl)-1-oxoisoindolin-2-yl)piperidine-2,6-dione 1 (78.0 mg, 285 umol, 1.00 eq) in N,N-dimethylformamide (2.00 mL) was added 4-dimethylaminopyridine (69.5 mg, 569 umol, 2.00 eq). The mixture was stirred at 50° C. for 1 h. The mixture was poured into water (20.0 mL) and extracted with ethyl acetate (3×10.0 mL). The organic phase was washed by saturated sodium chloride aqueous solution (20.0 mL) and brine (20.0 ml), dried over anhydrous sodium sulfate, filtered and concentrated under reduced pressure to give a residue. The residue was purified by Prep-HPLC (column: Phenomenex Synergi C18 150*25 mm*10 um; mobile phase: [water (formic acid)-acetonitrile]; B %: 31%-61%, 10 min) to afford (2-(2,6-dioxopiperidin-3-yl)-3-oxoisoindolin-5-yl)methyl(5-(1,1-difluoroethyl)-2-fluorophenyl)carbamate 434 (54.0 mg, 112 umol, 97% yield, 99% purity, formate) as a white solid. ¹H NMR (400 MHz, DMSO-d₆) δ=10.99 (s, 1H), 9.75 (s, 1H), 7.97-7.89 (m, 1H), 7.81 (s, 1H), 7.71-7.61 (m, 2H), 7.38-7.30 (m, 2H), 5.29 (s, 2H), 5.13 (dd, J=5.2, 13.6 Hz, 1H), 4.51-4.49 (m, 1H), 4.35-4.31 (m, 1H), 2.97-2.85 (m, 1H), 2.68-2.56 (m, 1H), 2.43-2.36 (m, 1H), 2.04-2.01 (m, 1H), 1.94 (t, J=18.8 Hz, 3H). MS (ESI-NEG) m/z 474.1 [M−H]⁺

Compound 435: To a mixture of 4-tert-butyl-1-chloro-2-nitro-benzene (2.00 g, 9.36 mmol, 1.00 eq) in sulfolane (40.0 mL) was added cesium fluoride (7.11 g, 46.8 mmol, 5.00 eq). The mixture was stirred at 180° C. for 3 h under microwave. The reaction mixture was poured into water (200 mL) and extracted with ethyl acetate (3×30 mL). The organic phase was concentrated under reduced pressure to give a residue. The residue was purified by column chromatography (SiO₂, petroleum ether/ethyl acetate=1/0) and concentrated to afford 4-tert-butyl-1-fluoro-2-nitro-benzene (600 mg, 1.83 mmol, 19% yield, 60% purity) as red oil. ¹H NMR (400 MHz, CDCl₃) δ=8.06-8.04 (m, 1H), 7.66-7.62 (m, 1H), 7.24-7.19 (m, 1H), 1.36 (s, 9H).

To a mixture of 4-tert-butyl-1-fluoro-2-nitro-benzene (500 mg, 1.52 mmol, 60% purity, 1.00 eq) in methanol (10.0 mL) was added palladium on carbon (50.0 mg, 10% purity) under nitrogen atmosphere. After addition, the mixture was purged with hydrogen for 3 times and stirred at 25° C. for 2 h under hydrogen atmosphere (15 psi). The reaction mixture was filtered and the filter cake was washed with methanol (3×30 mL). The filtrate was concentrated under reduced pressure to give a residue. The residue was purified by column chromatography (petroleum ether/ethyl acetate=1/0-50/1) to afford 5-tert-butyl-2-fluoro-aniline (200 mg, 956 umol, 62% yield, 80% purity) as red oil. ¹H NMR (400 MHz, CDCl₃) δ=6.97-6.89 (m, 2H), 6.82-6.77 (m, 1H), 1.28 (s, 9H).

To a mixture of 5-tert-butyl-2-fluoro-aniline (180 mg, 861 umol, 80% purity, 1.00 eq) and pyridine (102 mg, 1.29 mmol, 1.50 eq) in tetrahydrofuran (5.00 mL) was added phenyl carbonochloridate (141 mg, 904 umol, 1.05 eq) at 0° C. After stirring at 25° C. for 0.5 h, the reaction mixture was poured into saturated ammonium chloride aqueous (20 mL) and extracted with ethyl acetate (3×10 mL). The organic phase was concentrated under reduced pressure to give a residue. The residue was purified by column chromatography (petroleum ether/ethyl acetate=1/0-30/1) to afford phenyl N-(5-tert-butyl-2-fluoro-phenyl)carbamate (230 mg, 680 umol, 79% yield, 85% purity) as red oil. MS (ESI) m/z 288.0 [M+H]⁺

To a mixture of phenyl (5-(tert-butyl)-2-fluorophenyl)carbamate (100 mg, 296 umol, 85% purity, 1.20 eq) and 3-(6-(hydroxymethyl)-1-oxoisoindolin-2-yl)piperidine-2,6-dione 1 (67.6 mg, 247 umol, 1.00 eq) in N,N-dimethylformamide (2.00 mL) was added sodium hydride (14.8 mg, 370 umol, 60% purity, 1.50 eq) at 0° C. The mixture was stirred at 25° C. for 1.5 h. The mixture was quenched with hydrochloric acid (1 M, 1.00 mL), then diluted with ethyl acetate (1.00 mL). The aqueous phase was extracted with ethyl acetate (3×2 mL). The combined extracts were washed with brine, dried over anhydrous sodium sulfate, filtered and concentrated under vacuum to give a residue. The residue was purified by Prep-HPLC (column: Phenomenex Synergi C18 150*25 mm*10 um; mobile phase: [water (formic acid)-acetonitrile]; B %: 35%-68%, 11 min) to afford (2-(2,6-dioxopiperidin-3-yl)-3-oxoisoindolin-5-yl)methyl(5-(tert-butyl)-2-fluorophenyl)carbamate 435 (75.86 mg, 145 umol, 59% yield, 98% purity) as a white solid. ¹H NMR (400 MHz, DMSO-d₆) δ=10.99 (s, 1H), 9.44 (s, 1H), 8.50-8.43 (m, 1H), 7.81 (s, 1H), 7.73-7.60 (m, 3H), 7.20-7.07 (m, 2H), 5.27 (s, 2H), 5.19-5.09 (dd, J=5.2, 13.6 Hz, 1H), 4.52-4.44 (m, 1H), 4.39-4.31 (m, 1H), 2.98-2.87 (m, 1H), 2.69-2.57 (m, 1H), 2.42-2.32 (m, 1H), 2.09-1.95 (m, 1H), 1.25 (s, 9H). MS (ESI) m/z 468.2 [M+H]⁺

Compound 436: To a solution of dimethyl 2-methylmalonate (915 mg, 6.27 mmol, 832 uL, 1.10 eq) in dimethyl formamide (3.00 mL) was added sodium hydrogen (273 mg, 6.84 mmol, 60% purity, 1.20 eq) and the mixture was stirred at 0° C. for 0.5 h. Then to the mixture was added 2-chloro-1-fluoro-4-nitrobenzene (1.00 g, 5.70 mmol, 1.00 eq). The mixture was stirred at 25° C. for 2 h. The reaction was quenched by saturated ammonium chloride solution (50 mL), extracted with ethyl acetate (3×30 mL). The combined organic layers were concentrated to give a residue. The residue was purified by silica gel column chromatography (petroleum ether/ethyl acetate=10/1) to afford dimethyl 2-(2-chloro-4-nitrophenyl)-2-methylmalonate (5.00 g, 16.57 mmol, 97% yield) as yellow oil. ¹H NMR (400 MHz, DMSO-d₆) δ=8.32 (d, J=2.4 Hz, 1H), 8.18 (dd, J=2.5, 8.8 Hz, 1H), 7.60 (d, J=8.8 Hz, 1H), 3.74 (s, 6H), 1.86 (s, 3H).

To a mixture of dimethyl 2-(2-chloro-4-nitrophenyl)-2-methylmalonate (3.00 g, 9.94 mmol, 1.00 eq) in tetrahydrofuran (30.0 mL) were added lithium aluminium tetrahydride (400 mg, 10.5 mmol, 1.06 eq). The reaction mixture was stirred at 0° C. for 2 h under nitrogen atmosphere. The mixture was quenched with sodium sulfate decahydrate at 0° C. The mixture was filtered. The filtrate was concentrated to give a residue. The reaction was purified by silica gel column chromatography (petroleum ether/ethyl acetate=1/1) to afford 2-(2-chloro-4-nitro-phenyl)-2-methyl-propane-1,3-diol (500 mg, 2.04 mmol, 21% yield) as a yellow solid.

To a solution of 2-(2-chloro-4-nitrophenyl)-2-methylpropane-1,3-diol (500 mg, 2.04 mmol, 1.00 eq) in tetrahydrofuran (5.00 mL) was added n-butyllithium (2.50 M, 985 uL, 1.21 eq) and p-toluenesulfonyl chloride (582 mg, 3.05 mmol, 1.50 eq) at 0° C. The mixture was stirred at 25° C. for 1 h. After being cooled to 0° C., n-butyllithium (2.50 M, 985 uL, 1.21 eq) was added to the mixture. The mixture was stirred at 65° C. for 2 h. The reaction mixture was quenched by addition saturated ammonium chloride solution (10.0 mL), diluted with water (10.0 mL) and extracted with ethyl acetate (3×30.0 mL). The combined organic layers were dried over anhydrous sodium sulfate, filtered and concentrated under reduced pressure to give a residue. The residue was purified by chromatography on silica gel (petroleum ether/ethyl acetate=10/1 to dichloromethane/methanol=10/1) to afford 3-(2-chloro-4-nitro-phenyl)-3-methyloxetane (140 mg, 615 umol, 30% yield) as yellow oil. ¹H NMR (400 MHz, DMSO-d₆) δ=8.26 (d, J=2.4 Hz, 1H), 8.18 (dd, J=2.4, 8.5 Hz, 1H), 7.45 (d, J=8.4 Hz, 1H), 4.95 (d, J=6.0 Hz, 2H), 4.52 (d, J=6.4 Hz, 2H), 1.72 (s, 3H).

To a solution of 3-(2-chloro-4-nitrophenyl)-3-methyloxetane (140 mg, 615 umol, 1.00 eq) in methanol (2.00 mL) and water (1.00 mL) were added iron powder (103 mg, 1.84 mmol, 3.00 eq) and ammonium chloride (164 mg, 3.07 mmol, 5.00 eq). The mixture was stirred at 80° C. for 2 h. The mixture was diluted with water (20.0 mL) and extracted with ethyl acetate (3×20.0 mL). The combined organic layers were washed with brine (20.0 mL), dried over sodium sulfate, filtered and concentrated under reduced pressure to give a residue. The residue was purified by silica gel column chromatography (petroleum ether/ethyl acetate=1/1) to afford 3-chloro-4-(3-methyloxetan-3-yl)aniline (120 mg, 607 umol, 98% yield) as yellow oil. MS (ESI) m/z 198.1 [M+H]⁺

To a mixture of 3-chloro-4-(3-methyloxetan-3-yl)aniline (120 mg, 607 umol, 1.00 eq) and pyridine (144 mg, 1.82 mmol, 147 uL, 3.00 eq) in acetonitrile (1.00 mL) was added phenyl carbonochloridate (104 mg, 667 umol, 83.6 uL, 1.10 eq). The reaction was stirred at 25° C. for 2 h. The residue was diluted with water (20.0 mL) and extracted with ethyl acetate (3×20.0 mL). The combined organic layers were washed with brine (20.0 mL), dried over sodium sulfate, filtered and concentrated under reduced pressure to give a residue. The residue was purified by silica gel column chromatography (petroleum ether/ethyl acetate=10/1) to give phenyl (3-chloro-4-(3-methyloxetan-3-yl)phenyl)carbamate (80.0 mg, 251.7 umol, 42% yield) as yellow oil. ¹H NMR (400 MHz, DMSO-d₆) δ=10.40 (br s, 1H), 7.59 (d, J=2.0 Hz, 1H), 7.47-7.39 (m, 3H), 7.30-7.19 (m, 3H), 7.09 (d, J=8.4 Hz, 1H), 4.89 (d, J=5.6 Hz, 2H), 4.45 (d, J=6.0 Hz, 2H), 1.66 (s, 3H).

To a mixture of 3-(6-(hydroxymethyl)-1-oxoisoindolin-2-yl)piperidine-2,6-dione 1 (34.5 mg, 125 umol, 1.00 eq) and sodium hydrogen (10.0 mg, 251 umol, 60% purity, 2.00 eq) in dimethyl formamide (500 uL) was added phenyl (3-chloro-4-(3-methyloxetan-3-yl)phenyl)carbamate (40.0 mg, 126 umol, 1.00 eq). The reaction was stirred at 20° C. for 1 h. The pH of the mixture was adjusted to 7 with formic acid, then the mixture was diluted with dimethyl formamide (1.00 mL). The mixture was purified by Prep-HPLC (column: Phenomenex Luna C18 150*25 mm*10 um; mobile phase: [water (formic acid)-acetonitrile]; B %: 32%-62%, 10 min) to afford (2-(2,6-dioxopiperidin-3-yl)-3-oxoisoindolin-5-yl)methyl (3-chloro-4-(3-methyloxetan-3-yl)phenyl)carbamate 436 (27.58 mg, 54.8 umol, 44% yield, 99% purity, formate) as a white solid. ¹H NMR (400 MHz, DMSO-d₆) δ=10.98 (br s, 1H), 10.01 (s, 1H), 8.47 (s, 1H), 7.79 (s, 1H), 7.71-7.62 (m, 2H), 7.56 (d, J=1.6 Hz, 1H), 7.37 (dd, J=2.0, 8.4 Hz, 1H), 7.04 (d, J=8.4 Hz, 1H), 5.28 (s, 2H), 5.12 (dd, J=5.2, 13.3 Hz, 1H), 4.87 (d, J=5.6 Hz, 2H), 4.50-4.42 (m, 3H), 4.37-4.30 (m, 1H), 2.99-2.84 (m, 1H), 2.65-2.56 (m, 1H), 2.40 (dq, J=4.4, 13.2 Hz, 1H), 2.07-1.96 (m, 1H), 1.65 (s, 3H). MS (ESI) m/z 498.2 [M+H]⁺

Compound 437: To a solution of 5-chloro-4-methylpyridin-2-amine (2.00 g, 14.0 mmol, 1.00 eq) in sulfuric acid (30.0 mL) was added hydrogen peroxide (15.9 g, 140 mmol, 13.5 mL, 30% purity, 10.0 eq) dropwise at 0° C. After stirring at 25° C. for 12 h, the mixture was poured into ice water (60.0 mL), yellow solid was precipitate out. The mixture was filtered and the filter cake was dried under reduced pressure to afford 5-chloro-4-methyl-2-nitropyridine (2.00 g, 11.6 mmol, 83% yield) as a yellow solid. ¹H NMR (400 MHz, DMSO-d₆) δ=8.69 (s, 1H), 8.43 (s, 1H), 2.53 (s, 3H).

To a solution of 5-chloro-4-methyl-2-nitropyridine (1.00 g, 5.79 mmol, 1.00 eq) and (R)-2-methylpyrrolidine (1.97 g, 23.2 mmol, 4.00 eq) in N,N-dimethylformamide (10.0 mL) was added potassium carbonate (2.40 g, 17.4 mmol, 3.00 eq). After stirring at 80° C. for 12 h, the mixture was diluted with water (30.0 mL) and extracted with ethyl acetate (3×30 mL). The organic phase was washed with brine (100 mL), dried over anhydrous sodium sulfate, filtered and the filtrate was concentrated to give a residue. The residue was purified by flash silica gel chromatography (petroleum ether/ethylacetate=1/0 to 3/2) to afford (R)-4-methyl-5-(2-methylpyrrolidin-1-yl)-2-nitropyridine (340 mg, 1.38 mmol, 24% yield, 90% purity) as yellow oil. MS (ESI) m/z 222.0 [M+H]⁺

To a solution of (R)-4-methyl-5-(2-methylpyrrolidin-1-yl)-2-nitropyridine (340 mg, 1.54 mmol, 1.00 eq) in methanol (5.00 mL) was added palladium on carbon (34.0 mg, 10% purity) under nitrogen atmosphere. The suspension was degassed and purged with hydrogen atmosphere for 3 times. After stirring under hydrogen atmosphere (15 Psi) at 25° C. for 4 h, the reaction mixture was filtered and the filtrate was concentrated under reduced pressure to afford (R)-4-methyl-5-(2-methylpyrrolidin-1-yl)pyridin-2-amine (250 mg, 1.11 mmol, 72% yield, 85% purity) as yellow oil. MS (ESI) m/z 192.2 [M+H]⁺

To a mixture of (R)-4-methyl-5-(2-methylpyrrolidin-1-yl)pyridin-2-amine (150 mg, 784 umol, 1.00 eq) and pyridine (186 mg, 2.35 mmol, 190 uL, 3.00 eq) in acetonitrile (2.00 mL) was added phenyl carbonochloridate (111 mg, 706 umol, 88.4 uL, 0.900 eq) dropwise at 0° C. After stirring at 25° C. for 1 h, the mixture was concentrated under vacuum to give a residue. The residue was purified by Prep-TLC (SiO₂, petroleum ether/ethyl acetate=2/1) to afford (R)-phenyl (4-methyl-5-(2-methylpyrrolidin-1-yl)pyridin-2-yl)carbamate (65.0 mg, 188 umol, 24% yield, 90% purity) as yellow oil. MS (ESI) m/z 311.8 [M+H]⁺

To a mixture of (R)-phenyl (4-methyl-5-(2-methylpyrrolidin-1-yl)pyridin-2-yl)carbamate (60.0 mg, 193 umol, 1.00 eq) and 3-(6-(hydroxymethyl)-1-oxoisoindolin-2-yl)piperidine-2,6-dione 1 (52.9 mg, 193 umol, 1.00 eq) in dioxane (1.00 mL) were added 4-dimethylaminopyridine (23.5 mg, 193 umol, 1.00 eq) and N,N-diisopropylethylamine (29.9 mg, 231 umol, 40.3 uL, 1.20 eq) at 25° C. After stirring at 90° C. for 2 h, the reaction mixture was concentrated under reduced pressure to give a residue. The residue was purified by Prep-HPLC (column: Unisil 3-100 C18 Ultra 150*50 mm*3 um; mobile phase: [water (formic acid)-acetonitrile]; B %: 7%-37%, 10 min) to afford (2-(2,6-dioxopiperidin-3-yl)-3-oxoisoindolin-5-yl)methyl (4-methyl-5-((R)-2-methylpyrrolidin-1-yl)pyridin-2-yl)carbamate 437 (18.74 mg, 36.98 umol, 19% yield, 97% purity) as a white solid. ¹H NMR (400 MHz, DMSO-d₆) δ=10.98 (s, 1H), 10.01 (s, 1H), 8.49 (s, 1H), 7.87 (s, 1H), 7.79 (s, 1H), 7.68-7.61 (m, 2H), 7.59 (s, 1H), 5.27 (s, 2H), 5.12 (dd, J=5.2 Hz, 13.2 Hz, 1H), 4.49-4.44 (m, 1H), 4.36-4.31 (m, 1H), 3.70-3.64 (m, 1H), 3.50-3.40 (m, 1H), 2.95-2.86 (m, 1H), 2.75-2.66 (m, 1H), 2.62-2.61 (m, 1H), 2.42-2.37 (m, 1H), 2.21 (s, 3H), 2.13-2.01 (m, 2H), 1.88-1.73 (m, 2H), 1.51-1.46 (m, 1H), 0.91 (d, J=6.0 Hz, 3H). MS (ESI) m/z 492.0 [M+H]⁺

### Compound 438:

To a solution of 5-bromopicolinaldehyde (5.00 g, 26.9 mmol, 1.00 eq) in N,N-dimethylformamide (50.0 mL) was added trimethyl(trifluoromethyl)silane (4.59 g, 32.3 mmol, 1.20 eq) and potassium carbonate (7.43 g, 53.8 mmol, 2.00 eq) at 25° C. After stirring at 25° C. for 4 h, the reaction mixture was poured into water (50.0 mL) and then extracted with ethyl acetate (3×50.0 mL). The combined organic phase was washed with brine (3×20.0 mL), dried with anhydrous sodium sulfate, filtered and the filtrate was concentrated under reduced pressure to give a residue. The residue was purified by flash silica gel chromatography (petroleum ether/ethylacetate=1/0 to 5/1) to afford 1-(5-bromopyridin-2-yl)-2,2,2-trifluoroethanol (5.10 g, 19.7 mmol, 73% yield, 99% purity) as a yellow solid. ¹H NMR (400 MHz, DMSO-d₆) δ=8.73-8.72 (m, 1H), 8.18-8.15 (m, 1H), 7.61-7.58 (m, 1H), 7.15-7.13 (m, 1H), 5.18-5.11 (m, 1H).

To a mixture of 1-(5-bromopyridin-2-yl)-2,2,2-trifluoroethanol (5.10 g, 19.7 mmol, 99% purity, 1.00 eq), tert-butyl carbamate (3.47 g, 29.6 mmol, 1.50 eq), [2-(2-aminophenyl)phenyl]-methylsulfonyloxy-palladium; ditert-butyl-[2-(2,4,6-triisopropylphenyl)phenyl]phosphane (2.82 g, 3.55 mmol, 0.180 eq) in dioxane (60.0 mL) was added sodium tert-butoxide (5.69 g, 59.2 mmol, 3.00 eq) at 25° C., the mixture was degassed and purged with nitrogen atmosphere for 3 times. After stirring at 100° C. for 12 h, the reaction mixture was poured into water (60.0 mL) and then extracted with ethyl acetate (3×60.0 mL), the combined organic phase was washed with brine (3×30.0 mL), dried over anhydrous sodium sulfate, filtered and the filtrate was concentrated under reduced pressure to give a residue. The residue was purified by flash silica gel chromatography (petroleum ether/ethylacetate=1/0 to 2/3) to afford tert-butyl (6-(2,2,2-trifluoro-1-hydroxyethyl)pyridin-3-yl)carbamate (1.50 g, 4.77 mmol, 24% yield, 93% purity) as yellow oil. MS (ESI) m/z 293.0 [M+H]⁺.

A mixture of tert-butyl (6-(2,2,2-trifluoro-1-hydroxyethyl)pyridin-3-yl)carbamate (1.50 g, 4.77 mmol, 93% purity, 1.00 eq) in hydrochloric acid/ethyl acetate (4 M, 40.0 mL) was stirred at 25° C. for 2 h. The reaction mixture was filtered and the filter cake was dried under reduced pressure to give a residue, the residue was poured into saturated sodium bicarbonate solution (20.0 mL). The mixture was extracted with ethyl acetate (3×20.0 mL), the combined organic phase was washed with brine (3×10.0 mL), dried over anhydrous sodium sulfate, filtered and the filtrate was concentrated under reduced pressure to afford 1-(5-aminopyridin-2-yl)-2,2,2-trifluoroethanol (900 mg, 4.64 mmol, 97% yield, 99% purity) as a yellow solid. ¹H NMR (400 MHz, DMSO-d₆) δ=7.89-7.87 (m, 1H), 7.23-7.21 (m, 1H), 6.97-6.96 (m, 1H), 6.59-6.57 (m, 1H), 5.45 (s, 2H), 4.90-4.86 (m, 1H). MS (ESI) m/z 193.0 [M+H]⁺.

To a solution of 1-(5-aminopyridin-2-yl)-2,2,2-trifluoroethanol (400 mg, 2.06 mmol, 99% purity, 1.00 eq) in dichloromethane (8.00 mL) was added 2,6-dimethylpyridine (331 mg, 3.09 mmol, 360 uL, 1.50 eq) and tert-butyldimethylsilyl trifluoromethanesulfonate (654 mg, 2.47 mmol, 569 uL, 1.20 eq) at 0° C. After stirring at 25° C. for 12 h, the reaction mixture was concentrated under reduced pressure to give a residue. The residue was purified by flash silica gel chromatography (petroleum ether/ethylacetate=1/0 to 2/3) to afford 6-(1-((tert-butyldimethylsilyl)oxy)-2,2,2-trifluoroethyl)pyridin-3-amine (300 mg, 950 umol, 46% yield, 97% purity) as yellow oil. MS (ESI) m/z 307.1 [M+H]⁺.

To a solution of 6-(1-((tert-butyldimethylsilyl)oxy)-2,2,2-trifluoroethyl)pyridin-3-amine (250 mg, 791 umol, 97% purity, 1.00 eq) in tetrahydrofuran (3.00 mL) was added pyridine (125 mg, 1.58 mmol, 128 uL, 2.00 eq) and phenyl carbonochloridate (136 mg, 871 umol, 109 uL, 1.10 eq) at 25° C. After stirring at 25° C. for 2 h, the reaction mixture was concentrated under reduced pressure to give a residue. The residue was purified by flash silica gel chromatography (petroleum ether/ethylacetate=1/0 to 4/1) to afford phenyl (6-(1-((tert-butyldimethylsilyl)oxy)-2,2,2-trifluoroethyl)pyridin-3-yl)carbamate (360 mg, 777 umol, 98% yield, 92% purity) as a yellow solid. MS (ESI) m/z 427.2 [M+H]⁺.

To a solution of 3-(6-(hydroxymethyl)-1-oxoisoindolin-2-yl)piperidine-2,6-dione (300 mg, 1.09 mmol, 1.33 eq) in tetrahydrofuran (5.00 mL) was added sodium hydride (100 mg, 2.50 mmol, 60% purity, 3.05 eq) at 0° C. under nitrogen atmosphere. After stirring at 25° C. for 1 h under nitrogen atmosphere, the mixture was cooled to 0° C. A solution of phenyl (6-(1-((tert-butyldimethylsilyl)oxy)-2,2,2-trifluoroethyl)pyridin-3-yl) carbamate (350 mg, 821 umol, 1.00 eq) in tetrahydrofuran (5.00 mL) was added to the mixture at 0° C. under nitrogen atmosphere, then the resulting mixture was stirred at 25° C. for another 1 h. The residue was quenched by water (10.0 mL) and partitioned between ethyl acetate (40.0 mL) and brine (20.0 mL). The aqueous layer was extracted with ethyl acetate (2×40.0 mL). The combined organic layers were dried over sodium sulfate and evaporated to afford (2-(2,6-dioxopiperidin-3-yl)-3-oxoisoindolin-5-yl)methyl (6-(1-((tert-butyldimethylsilyl)oxy)-2,2,2-trifluoroethyl)pyridin-3-yl)carbamate (200 mg, crude) as yellow gum. MS (ESI) m/z 607.1 [M+H]⁺.

To a solution of (2-(2,6-dioxopiperidin-3-yl)-3-oxoisoindolin-5-yl)methyl(6-(1-((tert-butyldimethylsilyl)oxy)-2,2,2-trifluoroethyl)pyridin-3-yl)carbamate (180 mg, 297 umol, 1.00 eq) in tetrahydrofuran (5.00 mL) was added tetrabutylammonium fluoride (4 M, 2.00 mL, 27.0 eq), the reaction solution was degassed and purged under nitrogen atmosphere for three times. After stirring at 25° C. for 12 h, the mixture was extracted between ethyl acetate (50.0 mL) and brine (4×20.0 mL). The combined organic layers were dried over sodium sulfate, filtered and the filtrate was evaporated to give a residue, which was purified by prep-HPLC (column: Unisil 3-100 C18 Ultra 150*50 mm*3 um; mobile phase: [water (FA)-ACN]; B %: 14%-44%, 10 min) for three times and lyophilized to afford (2-(2,6-dioxopiperidin-3-yl)-3-oxoisoindolin-5-yl)methyl(6-(2,2,2-trifluoro-1-hydroxyethyl)pyridin-3-yl)carbamate 438: (60 mg, 122 umol, 41% yield, 99% purity) as a white solid. ¹H NMR (400 MHz, DMSO-d₆) δ=10.98 (s, 1H), 10.16 (s, 1H), 8.62-8.61 (m, 1H), 7.98-7.70 (m, 1H), 7.68 (s, 1H), 7.63-7.53 (m, 2H), 7.55-7.53 (m, 1H), 6.95 (s, 1H), 5.30 (s, 2H), 5.12 (dd, J=5.2 Hz, 13.2 Hz, 1H), 5.05 (d, J=7.2 Hz, 1H), 4.47 (d, J=17.2 Hz, 1H), 4.47 (d, J=17.6 Hz, 1H), 2.91-2.86 (m, 1H), 2.63-2.58 (m, 1H), 2.46-2.40 (m, 1H), 2.03-2.01 (m, 1H). MS (ESI) m/z 493.2 [M+H]⁺.

Compound 439: To a mixture of 3-chloroaniline (500 mg, 3.92 mmol, 416 uL, 1.00 eq) and pyridine (930 mg, 11.7 mmol, 949 uL, 3.00 eq) in acetonitrile (5.00 mL) was added phenyl carbonochloridate (797 mg, 5.10 mmol, 638 uL, 1.30 eq) dropwise. The mixture was stirred at 15° C. for 2 h. The mixture was concentrated to give crude product. The crude product was purified by reversed-phase HPLC (column: spherical C18, 20-45 um, 100 Å, SW 80, mobile phase: [water (0.1% Formic Acid)-ACN]). The desired fraction was collected and lyophilized to give phenyl (3-chlorophenyl)carbamate (900 mg, 3.63 mmol, 92% yield) as a white solid. ¹H NMR (400 MHz, DMSO-d₆) δ=10.46 (br s, 1H), 7.51-7.45 (m, 2H), 7.45-7.41 (m, 2H), 7.40-7.37 (m, 1H), 7.36-7.32 (m, 1H), 7.28 (br d, J=7.5 Hz, 1H), 7.26-7.21 (m, 2H).

To a mixture of 3-(6-(hydroxymethyl)-1-oxoisoindolin-2-yl)piperidine-2,6-dione 1 (80.0 mg, 291 umol, 1.00 eq) and phenyl (3-chlorophenyl)carbamate (86.6 mg, 350 umol, 1.20 eq) in dimethyl formamide (1.00 mL) was added sodium hydride (17.5 mg, 437 umol, 60% purity, 1.50 eq) in one portion at 0° C. The mixture was stirred at 15° C. for 1 h. The mixture was quenched with 1 M hydrochloric acid (0.500 mL) and filtered. The filtrate was purified by prep-HPLC (column: 3_Phenomenex Luna C18 75*30 mm*3 um; mobile phase: [water (0.05% HCl)-ACN]; B %: 35%-55%, 6.5 min) and lyophilization to give (2-(2,6-dioxopiperidin-3-yl)-3-oxoisoindolin-5-yl)methyl (3-chlorophenyl)carbamate 439 (10.72 mg, 24.8 umol, 8% yield, 99% purity) as an off-white solid. ¹H NMR (400 MHz, DMSO-d₆) δ=11.00 (s, 1H), 10.05 (s, 1H), 7.80 (s, 1H), 7.70-7.67 (m, 1H), 7.66-7.59 (m, 2H), 7.41-7.36 (m, 1H), 7.33-7.27 (m, 1H), 7.07-7.03 (m, 1H), 5.28 (s, 2H), 5.13 (dd, J=5.1, 13.4 Hz, 1H), 4.51-4.43 (m, 1H), 4.38-4.30 (m, 1H), 2.97-2.85 (m, 1H), 2.60 (br d, J=17.7 Hz, 1H), 2.40-2.28 (m, 1H), 2.06-1.96 (m, 1H). MS (ESI) m/z 428.1 [M+H]⁺

Compound 440: To a solution of 1,2-difluoro-4-nitrobenzene (3.00 g, 18.9 mmol, 2.08 mL, 1.00 eq) in tetrahydrofuran (30.0 mL) was added sodium hydride (1.51 g, 37.7 mmol, 60% purity, 2.00 eq) slowly at 0° C., then stirred at 0° C. for 0.5 h. Dimethyl 2-methylmalonate (4.13 g, 28.3 mmol, 3.76 mL, 1.50 eq) was added to the mixture, the mixture was stirred at 25° C. for 3 h. The reaction mixture was poured into water (200 mL) slowly, then extracted with ethyl acetate (3×100 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=1/0 to 10/1) to afford dimethyl 2-(2-fluoro-4-nitrophenyl)-2-methylmalonate (3.80 g, 13.3 mmol, 71% yield) as yellow oil. ¹H NMR (400 MHz, DMSO-d₆) δ=8.16 (dd, J=2.3, 11.2 Hz, 1H), 8.08 (dd, J=2.3, 8.8 Hz, 1H), 7.65 (t, J=8.4 Hz, 1H), 3.74 (s, 6H), 1.83 (s, 3H).

To a solution of dimethyl 2-(2-fluoro-4-nitrophenyl)-2-methylmalonate (2.80 g, 9.82 mmol, 1.00 eq) in tetrahydrofuran (30.0 mL) was added lithium aluminum hydride (559 mg, 14.7 mmol, 1.50 eq) slowly at 0° C. under nitrogen atmosphere. Then the mixture was stirred at 0° C. for 3 h. The reaction mixture was added sodium sulfate decahydrate (10.0 g) slowly, then filtered to give a filtrate, the filtrate was concentrated to give a residue. The residue was purified by silica gel chromatography (Petroleum ether/Ethyl acetate=1/0 to 2/1) to afford 2-(2-fluoro-4-nitrophenyl)-2-methylpropane-1,3-diol (300 mg, 1.31 mmol, 13% yield) as yellow oil. ¹H NMR (400 MHz, DMSO-d₆) δ=8.12-7.95 (m, 2H), 7.68 (t, J=8.4 Hz, 1H), 4.76 (t, J=5.2 Hz, 2H), 3.82-3.70 (m, 2H), 3.67-3.58 (m, 2H), 1.30 (d, J=1.2 Hz, 3H).

To a solution of 2-(2-fluoro-4-nitrophenyl)-2-methylpropane-1,3-diol (500 mg, 2.18 mmol, 1.00 eq) in tetrahydrofuran (2.00 mL) was added n-butyllithium (2.50 M, 1.31 mL, 1.50 eq) and paratoluensulfonyl chloride (624 mg, 3.27 mmol, 1.50 eq) in 0° C. The mixture was stirred at 25° C. for 1 h and then stirred at 65° C. for 2 h. The reaction mixture was cooled to 25° C., then quenched with ammonium chloride (30.0 mL), then poured into water (80.0 mL). The aqueous phase was extracted with ethyl acetate (3×60.0 mL). The combined organic phase was washed with brine (60.0 mL), dried with anhydrous sodium sulfate, filtered and concentrated in vacuum to give a residue. The residue was purified by silica gel chromatography (Petroleum ether/Ethyl acetate=1/0 to 3/1) to afford 3-(2-fluoro-4-nitrophenyl)-3-methyloxetane (200 mg, 947 umol, 43% yield) as a yellow solid. ¹H NMR (400 MHz, CDCl₃) δ=8.13-8.02 (m, 1H), 7.94 (dd, J=2.3, 10.4 Hz, 1H), 7.19 (t, J=8.0 Hz, 1H), 5.05 (d, J=5.6 Hz, 2H), 4.65 (d, J=6.0 Hz, 2H), 1.79 (s, 3H).

To a solution of 3-(2-fluoro-4-nitrophenyl)-3-methyloxetane (200 mg, 947 umol, 1.00 eq) in methanol (2.00 mL) and water (1.00 mL) was added iron powder (159 mg, 2.84 mmol, 3.00 eq) and ammonium chloride (253 mg, 4.74 mmol, 5.00 eq). Then the mixture was stirred at 80° C. for 2 h. The mixture was filtered to give a filtrate. The filtrate was diluted with water (100 mL) and extracted with ethyl acetate (3×100 mL), washed with brine (200 mL), dried over anhydrous sodium sulfate, filtered and concentrated under reduced pressure to give 3-fluoro-4-(3-methyloxetan-3-yl)aniline (100 mg, 552 umol, 58% yield) as yellow oil. MS (ESI) m/z 182.2 [M+H]

To a solution of 3-fluoro-4-(3-methyloxetan-3-yl)aniline (100 mg, 551 umol, 1.00 eq) and pyridine (218 mg, 2.76 mmol, 222 uL, 5.00 eq) in acetonitrile (2.00 mL) was added phenyl carbonochloridate (95.0 mg, 607 umol, 76.0 uL, 1.10 eq) at 25° C. for 2 h. The reaction was concentrated under reduced pressure to give a residue. The crude product was purified by reversed-phase HPLC (0.1% formic acid condition) to afford phenyl (3-fluoro-4-(3-methyloxetan-3-yl)phenyl)carbamate as a white solid. ¹H NMR (400 MHz, DMSO-d₆) δ=10.53-10.33 (m, 1H), 7.50-7.38 (m, 2H), 7.31-7.21 (m, 3H), 7.19-7.13 (m, 2H), 6.38-6.29 (m, 1H), 4.86 (d, J=5.6 Hz, 2H), 4.48 (d, J=5.6 Hz, 2H), 1.62 (s, 3H). MS (ESI) m/z 302.0 [M+H]

To a solution of 3-(6-(hydroxymethyl)-1-oxoisoindolin-2-yl)piperidine-2,6-dione I (20.0 mg, 72.9 umol, 1.00 eq) and phenyl(3-fluoro-4-(3-methyloxetan-3-yl)phenyl)carbamate (32.9 mg, 109 umol, 1.50 eq) in dimethylformamide (1.00 mL) was added sodium hydride (5.83 mg, 145 umol, 60% purity, 2.00 eq) at 0° C., The reaction mixture was stirred at 25° C. for 1 h. The reaction mixture quenched with 1 M hydrochloric acid (500 uL) and filtered to give a filtrate. The residue was purified by Prep-HPLC (column: Shim-pack C18 150*25*10 um; mobile phase: [water (formic acid)-acetonitrile]; B %: 28%-58%, 10 min) to afford (2-(2,6-dioxopiperidin-3-yl)-3-oxoisoindolin-5-yl)methyl (3-fluoro-4-(3-methyloxetan-3-yl)phenyl)carbamate 440 (37.21 mg, 76.5 umol, 49% yield, 99% purity) as a white solid. ¹H NMR (400 MHz, DMSO-d₆) δ=10.99 (br d, J=2.4 Hz, 1H), 10.03 (s, 1H), 7.80 (s, 1H), 7.74-7.60 (m, 2H), 7.35 (br d, J=13.2 Hz, 1H), 7.22 (dd, J=1.6, 8.4 Hz, 1H), 7.13-7.02 (m, 1H), 5.29 (s, 2H), 5.13 (dd, J=5.0, 13.2 Hz, 1H), 4.85 (d, J=5.6 Hz, 2H), 4.51-4.47 (m, 1H), 4.46 (s, 2H), 4.41-4.29 (m, 1H), 3.00-2.85 (m, 1H), 2.65-2.59 (m, 1H), 2.44-2.35 (m, 1H), 2.09-1.95 (m, 1H), 1.61 (s, 3H). MS (ESI) m/z 482.1 [M+H]

TABLE 3
Specific examples 501-573

| Compound | No. |
|---|---|
| 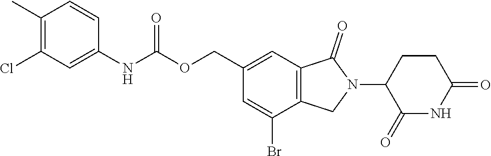 [SMILES: Cc1ccc(NC(=O)OCc2cc(Br)c3c(c2)C(=O)N(C2CCC(=O)NC2=O)C3)cc1Cl] | 501 |
| 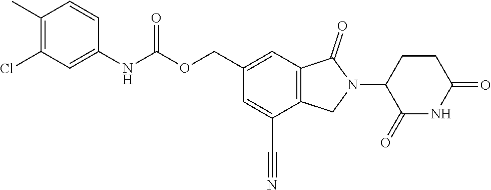 [SMILES: Cc1ccc(NC(=O)OCc2cc(C#N)c3c(c2)C(=O)N(C2CCC(=O)NC2=O)C3)cc1Cl] | 502 |
| 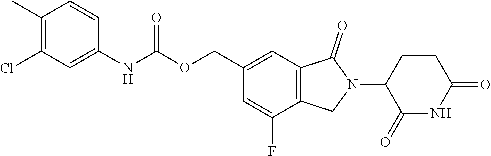 [SMILES: Cc1ccc(NC(=O)OCc2cc(F)c3c(c2)C(=O)N(C2CCC(=O)NC2=O)C3)cc1Cl] | 503 |
| 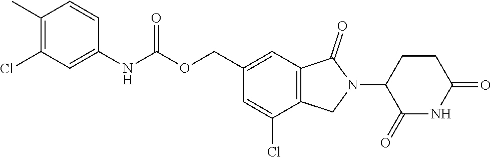 [SMILES: Cc1ccc(NC(=O)OCc2cc(Cl)c3c(c2)C(=O)N(C2CCC(=O)NC2=O)C3)cc1Cl] | 504 |
| 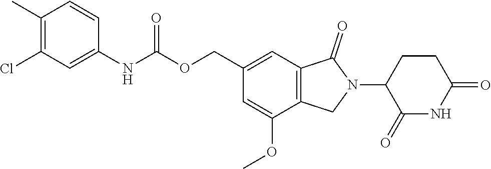 [SMILES: COc1cc(COC(=O)Nc2ccc(C)c(Cl)c2)cc2c1CN(C1CCC(=O)NC1=O)C2=O] | 505 |
| 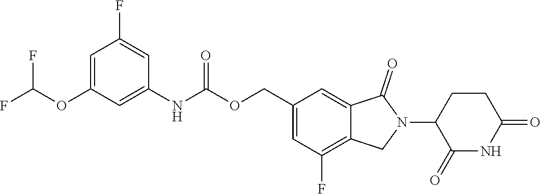 [SMILES: O=C1CCC(N2Cc3c(F)cc(COC(=O)Nc4cc(F)cc(OC(F)F)c4)cc3C2=O)C(=O)N1] | 506 |
| 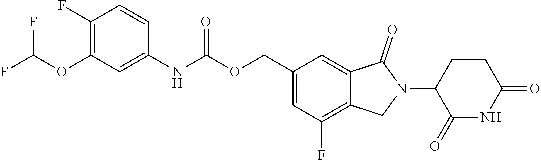 [SMILES: O=C1CCC(N2Cc3c(F)cc(COC(=O)Nc4ccc(F)c(OC(F)F)c4)cc3C2=O)C(=O)N1] | 507 |
| 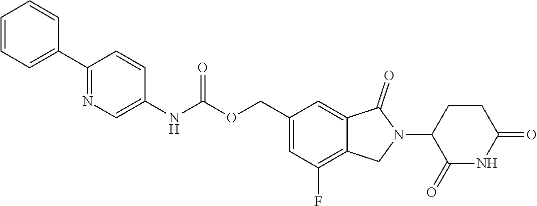 [SMILES: O=C1CCC(N2Cc3c(F)cc(COC(=O)Nc4ccc(-c5ccccc5)nc4)cc3C2=O)C(=O)N1] | 508 |
| 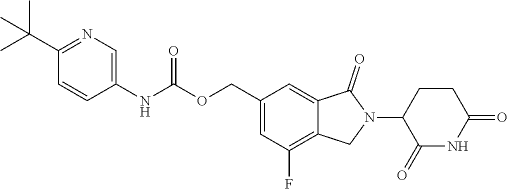 [SMILES: CC(C)(C)c1ccc(NC(=O)OCc2cc(F)c3c(c2)C(=O)N(C2CCC(=O)NC2=O)C3)cn1] | 509 |
| 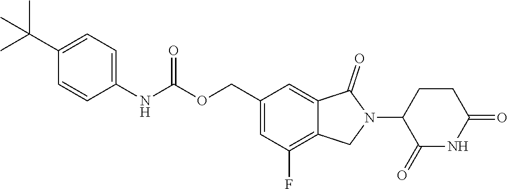 [SMILES: CC(C)(C)c1ccc(NC(=O)OCc2cc(F)c3c(c2)C(=O)N(C2CCC(=O)NC2=O)C3)cc1] | 510 |
| 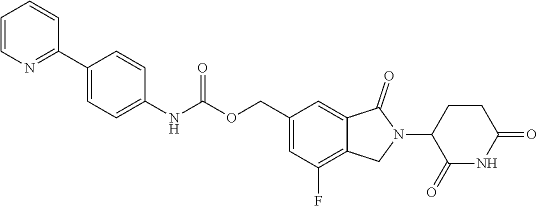 [SMILES: O=C1CCC(N2Cc3c(F)cc(COC(=O)Nc4ccc(-c5ccccn5)cc4)cc3C2=O)C(=O)N1] | 511 |
| 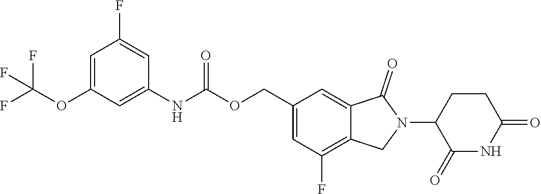 [SMILES: O=C1CCC(N2Cc3c(F)cc(COC(=O)Nc4cc(F)cc(OC(F)(F)F)c4)cc3C2=O)C(=O)N1] | 512 |
| 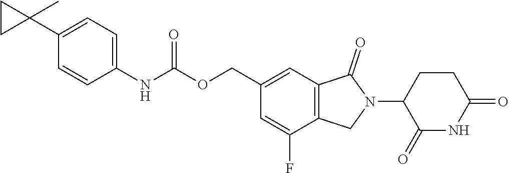 [SMILES: CC1(c2ccc(NC(=O)OCc3cc(F)c4c(c3)C(=O)N(C3CCC(=O)NC3=O)C4)cc2)CC1] | 513 |
| 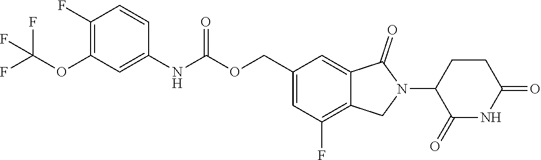 [SMILES: O=C1CCC(N2Cc3c(F)cc(COC(=O)Nc4ccc(F)c(OC(F)(F)F)c4)cc3C2=O)C(=O)N1] | 514 |
| 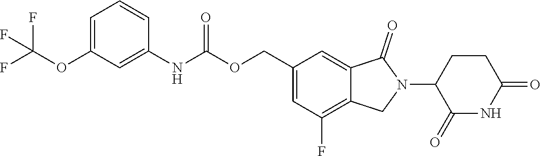 [SMILES: O=C1CCC(N2Cc3c(F)cc(COC(=O)Nc4cccc(OC(F)(F)F)c4)cc3C2=O)C(=O)N1] | 515 |
| 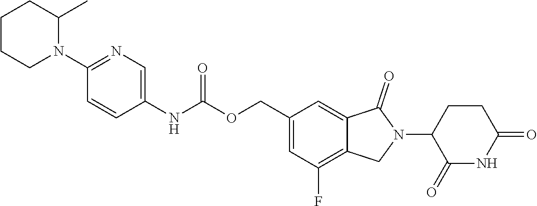 [SMILES: CC1CCCCN1c1ccc(NC(=O)OCc2cc(F)c3c(c2)C(=O)N(C2CCC(=O)NC2=O)C3)cn1] | 516 |
| 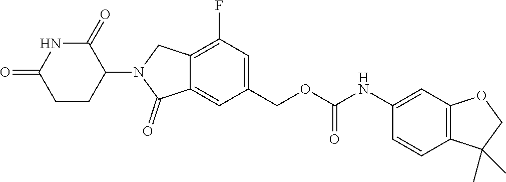 [SMILES: CC1(C)COc2cc(NC(=O)OCc3cc(F)c4c(c3)C(=O)N(C3CCC(=O)NC3=O)C4)ccc21] | 517 |
| 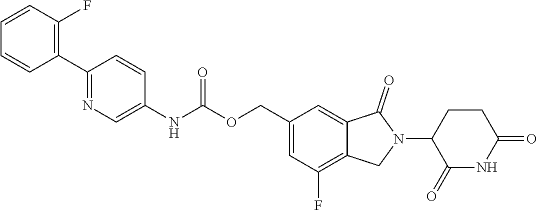 [SMILES: O=C1CCC(N2Cc3c(F)cc(COC(=O)Nc4ccc(-c5ccccc5F)nc4)cc3C2=O)C(=O)N1] | 518 |
| 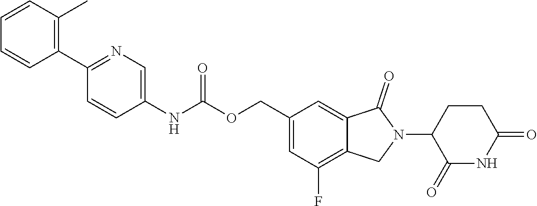 [SMILES: Cc1ccccc1-c1ccc(NC(=O)OCc2cc(F)c3c(c2)C(=O)N(C2CCC(=O)NC2=O)C3)cn1] | 519 |
| 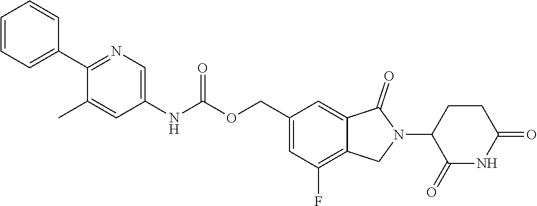 [SMILES: Cc1cc(NC(=O)OCc2cc(F)c3c(c2)C(=O)N(C2CCC(=O)NC2=O)C3)cnc1-c1ccccc1] | 520 |
| 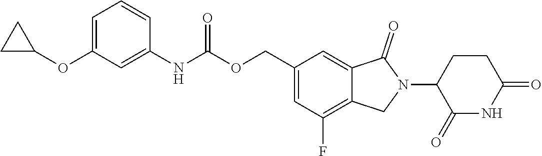 [SMILES: O=C1CCC(N2Cc3c(F)cc(COC(=O)Nc4cccc(OC5CC5)c4)cc3C2=O)C(=O)N1] | 521 |
| 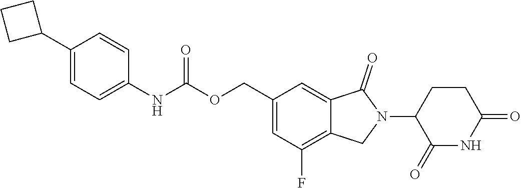 [SMILES: O=C1CCC(N2Cc3c(F)cc(COC(=O)Nc4ccc(C5CCC5)cc4)cc3C2=O)C(=O)N1] | 522 |
| 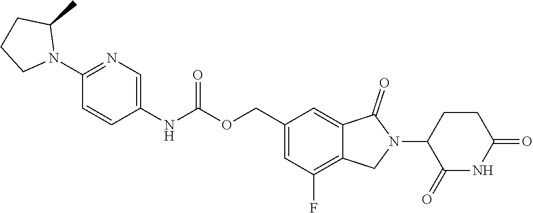 [SMILES: C[C@@H]1CCCN1c1ccc(NC(=O)OCc2cc(F)c3c(c2)C(=O)N(C2CCC(=O)NC2=O)C3)cn1] | 523 |
| 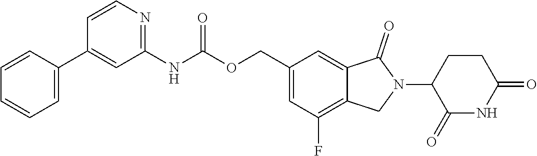 [SMILES: O=C1CCC(N2Cc3c(F)cc(COC(=O)Nc4cc(-c5ccccc5)ccn4)cc3C2=O)C(=O)N1] | 524 |
| 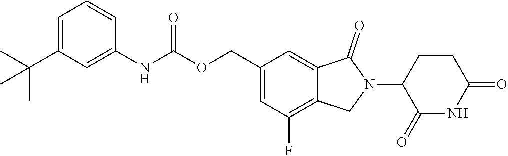 [SMILES: CC(C)(C)c1cccc(NC(=O)OCc2cc(F)c3c(c2)C(=O)N(C2CCC(=O)NC2=O)C3)c1] | 525 |
| 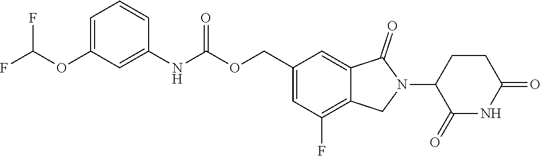 [SMILES: O=C1CCC(N2Cc3c(F)cc(COC(=O)Nc4cccc(OC(F)F)c4)cc3C2=O)C(=O)N1] | 526 |
| 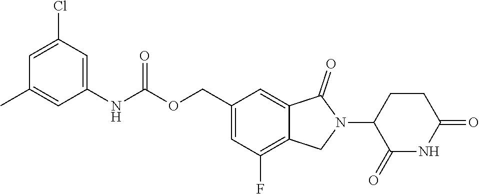 [SMILES: Cc1cc(Cl)cc(NC(=O)OCc2cc(F)c3c(c2)C(=O)N(C2CCC(=O)NC2=O)C3)c1] | 527 |
| 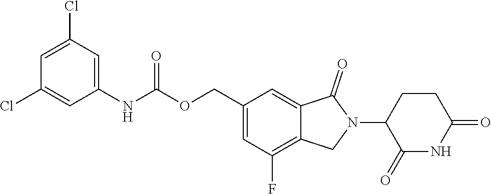 [SMILES: O=C1CCC(N2Cc3c(F)cc(COC(=O)Nc4cc(Cl)cc(Cl)c4)cc3C2=O)C(=O)N1] | 528 |
| 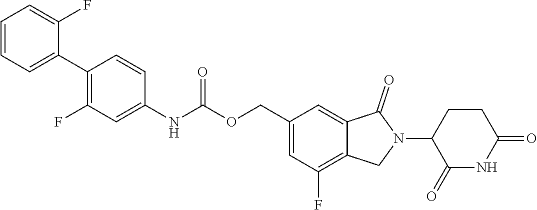 [SMILES: O=C1CCC(N2Cc3c(F)cc(COC(=O)Nc4ccc(-c5ccccc5F)c(F)c4)cc3C2=O)C(=O)N1] | 529 |
| 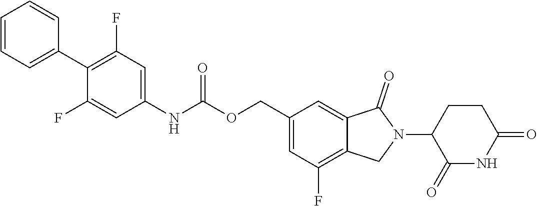 [SMILES: O=C1CCC(N2Cc3c(F)cc(COC(=O)Nc4cc(F)c(-c5ccccc5)c(F)c4)cc3C2=O)C(=O)N1] | 530 |
| 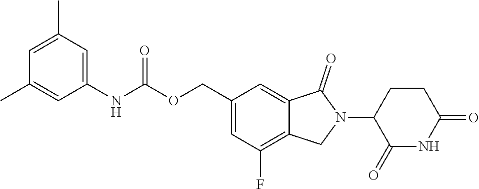 [SMILES: Cc1cc(C)cc(NC(=O)OCc2cc(F)c3c(c2)C(=O)N(C2CCC(=O)NC2=O)C3)c1] | 531 |
| 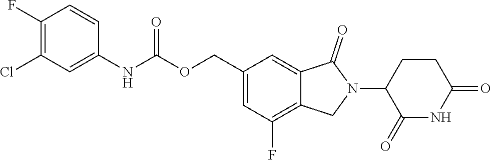 [SMILES: O=C1CCC(N2Cc3c(F)cc(COC(=O)Nc4ccc(F)c(Cl)c4)cc3C2=O)C(=O)N1] | 532 |
| 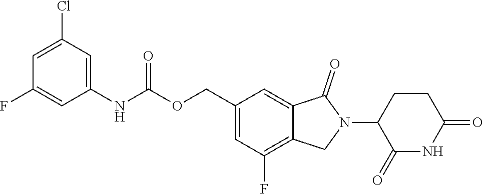 [SMILES: O=C1CCC(N2Cc3c(F)cc(COC(=O)Nc4cc(F)cc(Cl)c4)cc3C2=O)C(=O)N1] | 533 |
| 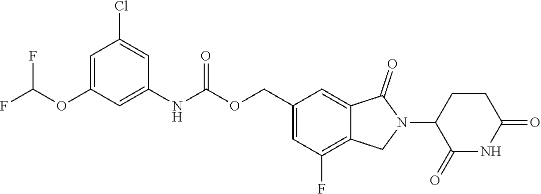 [SMILES: O=C1CCC(N2Cc3c(F)cc(COC(=O)Nc4cc(Cl)cc(OC(F)F)c4)cc3C2=O)C(=O)N1] | 534 |
| 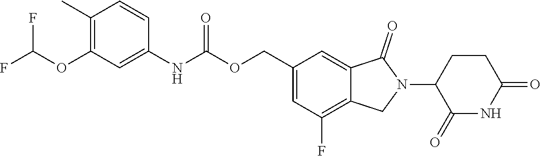 [SMILES: Cc1ccc(NC(=O)OCc2cc(F)c3c(c2)C(=O)N(C2CCC(=O)NC2=O)C3)cc1OC(F)F] | 535 |
| 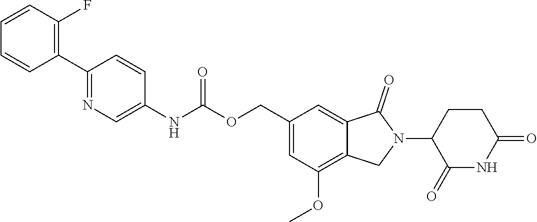 [SMILES: COc1cc(COC(=O)Nc2ccc(-c3ccccc3F)nc2)cc2c1CN(C1CCC(=O)NC1=O)C2=O] | 536 |
| 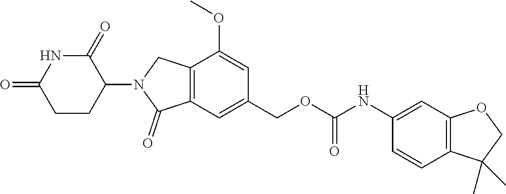 [SMILES: COc1cc(COC(=O)Nc2ccc3c(c2)OCC3(C)C)cc2c1CN(C1CCC(=O)NC1=O)C2=O] | 537 |
| 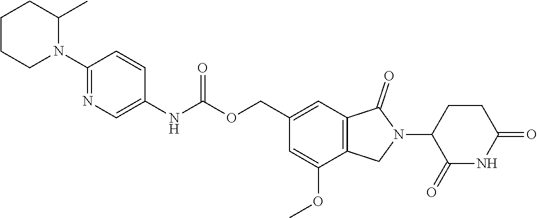 [SMILES: COc1cc(COC(=O)Nc2ccc(N3CCCCC3C)nc2)cc2c1CN(C1CCC(=O)NC1=O)C2=O] | 538 |
| 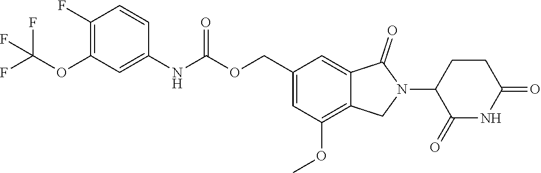 [SMILES: COc1cc(COC(=O)Nc2ccc(F)c(OC(F)(F)F)c2)cc2c1CN(C1CCC(=O)NC1=O)C2=O] | 539 |
| 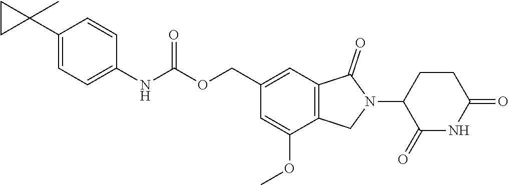 [SMILES: COc1cc(COC(=O)Nc2ccc(C3(C)CC3)cc2)cc2c1CN(C1CCC(=O)NC1=O)C2=O] | 540 |
| 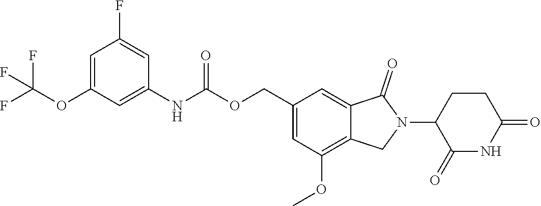 [SMILES: COc1cc(COC(=O)Nc2cc(F)cc(OC(F)(F)F)c2)cc2c1CN(C1CCC(=O)NC1=O)C2=O] | 541 |
| 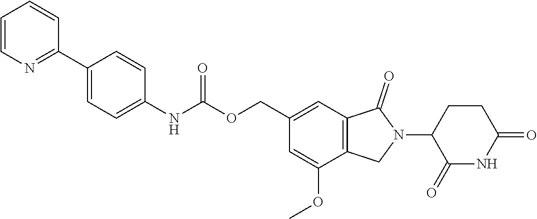 [SMILES: COc1cc(COC(=O)Nc2ccc(-c3ccccn3)cc2)cc2c1CN(C1CCC(=O)NC1=O)C2=O] | 542 |
| 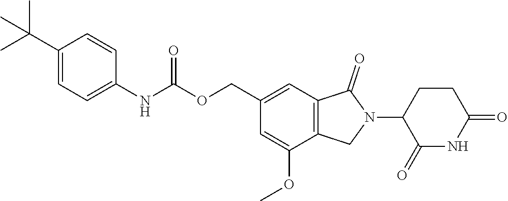 [SMILES: COc1cc(COC(=O)Nc2ccc(C(C)(C)C)cc2)cc2c1CN(C1CCC(=O)NC1=O)C2=O] | 543 |
| 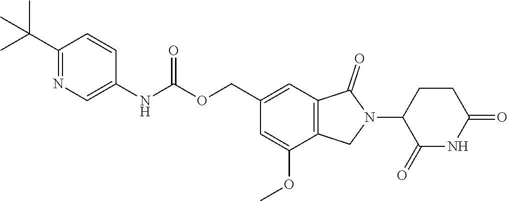 [SMILES: COc1cc(COC(=O)Nc2ccc(C(C)(C)C)nc2)cc2c1CN(C1CCC(=O)NC1=O)C2=O] | 44 |
| 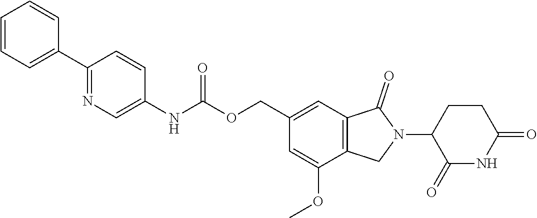 [SMILES: COc1cc(COC(=O)Nc2ccc(-c3ccccc3)nc2)cc2c1CN(C1CCC(=O)NC1=O)C2=O] | 545 |
| 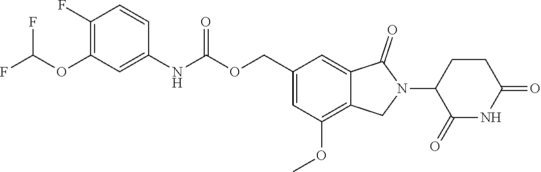 [SMILES: COc1cc(COC(=O)Nc2ccc(F)c(OC(F)F)c2)cc2c1CN(C1CCC(=O)NC1=O)C2=O] | 546 |
| 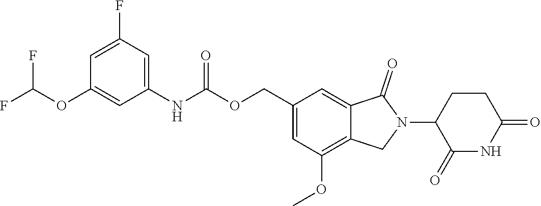 [SMILES: COc1cc(COC(=O)Nc2cc(F)cc(OC(F)F)c2)cc2c1CN(C1CCC(=O)NC1=O)C2=O] | 547 |
| 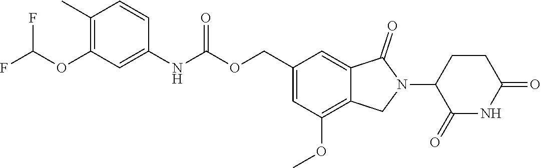 [SMILES: COc1cc(COC(=O)Nc2ccc(C)c(OC(F)F)c2)cc2c1CN(C1CCC(=O)NC1=O)C2=O] | 548 |
| 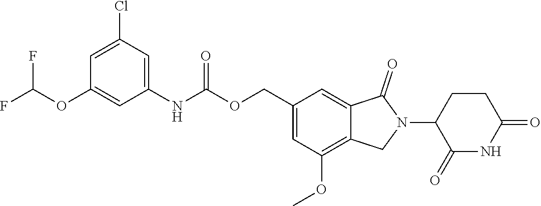 [SMILES: COc1cc(COC(=O)Nc2cc(Cl)cc(OC(F)F)c2)cc2c1CN(C1CCC(=O)NC1=O)C2=O] | 549 |
| 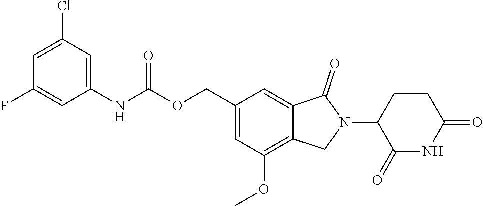 [SMILES: COc1cc(COC(=O)Nc2cc(F)cc(Cl)c2)cc2c1CN(C1CCC(=O)NC1=O)C2=O] | 550 |
| 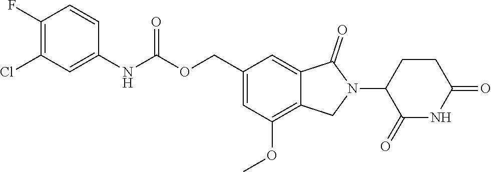 [SMILES: COc1cc(COC(=O)Nc2ccc(F)c(Cl)c2)cc2c1CN(C1CCC(=O)NC1=O)C2=O] | 551 |
| 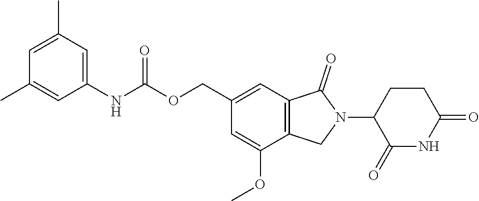 [SMILES: COc1cc(COC(=O)Nc2cc(C)cc(C)c2)cc2c1CN(C1CCC(=O)NC1=O)C2=O] | 552 |
| 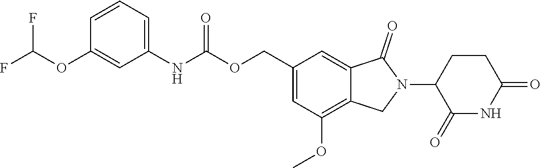 [SMILES: COc1cc(COC(=O)Nc2cccc(OC(F)F)c2)cc2c1CN(C1CCC(=O)NC1=O)C2=O] | 553 |
| 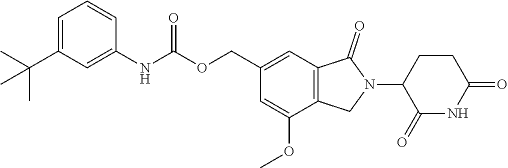 [SMILES: COc1cc(COC(=O)Nc2cccc(C(C)(C)C)c2)cc2c1CN(C1CCC(=O)NC1=O)C2=O] | 554 |
| 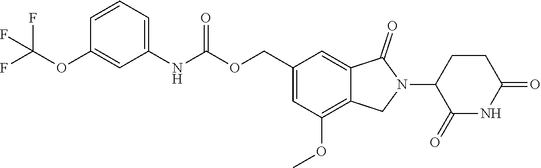 [SMILES: COc1cc(COC(=O)Nc2cccc(OC(F)(F)F)c2)cc2c1CN(C1CCC(=O)NC1=O)C2=O] | 555 |
| 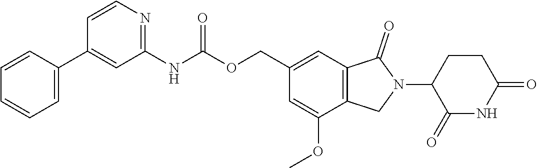 [SMILES: COc1cc(COC(=O)Nc2cc(-c3ccccc3)ccn2)cc2c1CN(C1CCC(=O)NC1=O)C2=O] | 556 |
| 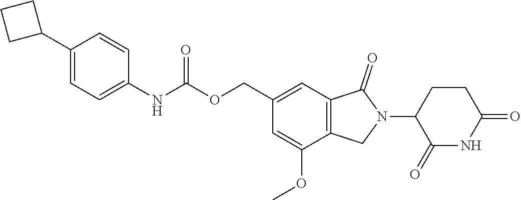 [SMILES: COc1cc(COC(=O)Nc2ccc(C3CCC3)cc2)cc2c1CN(C1CCC(=O)NC1=O)C2=O] | 557 |
| 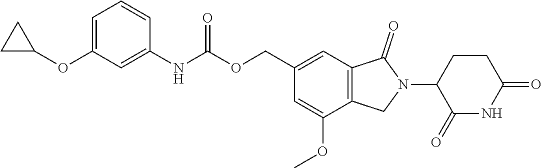 [SMILES: COc1cc(COC(=O)Nc2cccc(OC3CC3)c2)cc2c1CN(C1CCC(=O)NC1=O)C2=O] | 558 |
| 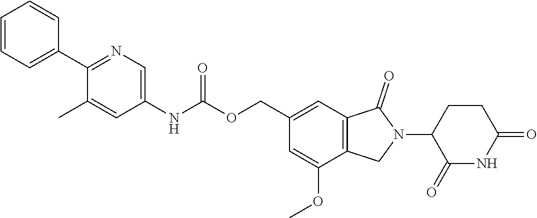 [SMILES: COc1cc(COC(=O)Nc2cnc(-c3ccccc3)c(C)c2)cc2c1CN(C1CCC(=O)NC1=O)C2=O] | 559 |
| 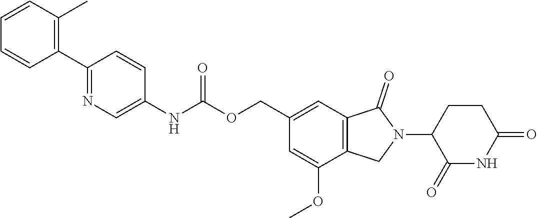 [SMILES: COc1cc(COC(=O)Nc2ccc(-c3ccccc3C)nc2)cc2c1CN(C1CCC(=O)NC1=O)C2=O] | 560 |
| 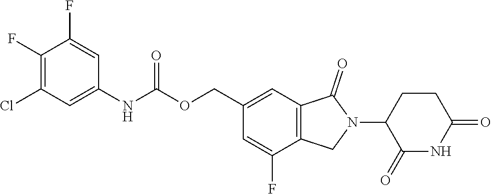 [SMILES: O=C1CCC(N2Cc3c(F)cc(COC(=O)Nc4cc(F)c(F)c(Cl)c4)cc3C2=O)C(=O)N1] | 561 |
| 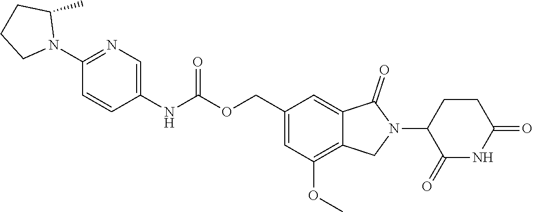 [SMILES: COc1cc(COC(=O)Nc2ccc(N3CCC[C@@H]3C)nc2)cc2c1CN(C1CCC(=O)NC1=O)C2=O] | 562 |
| 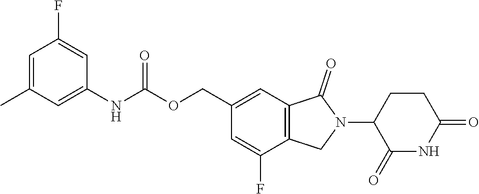 [SMILES: Cc1cc(F)cc(NC(=O)OCc2cc(F)c3c(c2)C(=O)N(C2CCC(=O)NC2=O)C3)c1] | 563 |
| 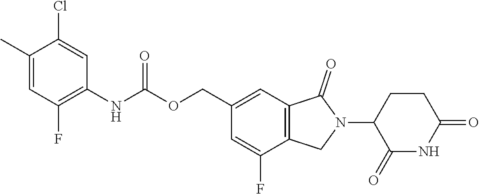 [SMILES: Cc1cc(F)c(NC(=O)OCc2cc(F)c3c(c2)C(=O)N(C2CCC(=O)NC2=O)C3)cc1Cl] | 564 |
| 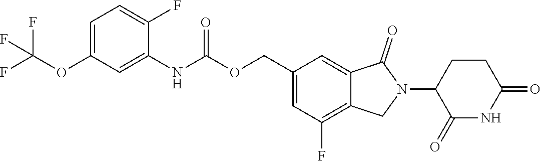 [SMILES: O=C1CCC(N2Cc3c(F)cc(COC(=O)Nc4cc(OC(F)(F)F)ccc4F)cc3C2=O)C(=O)N1] | 565 |
| 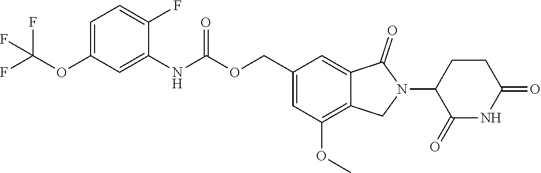 [SMILES: COc1cc(COC(=O)Nc2cc(OC(F)(F)F)ccc2F)cc2c1CN(C1CCC(=O)NC1=O)C2=O] | 566 |
| 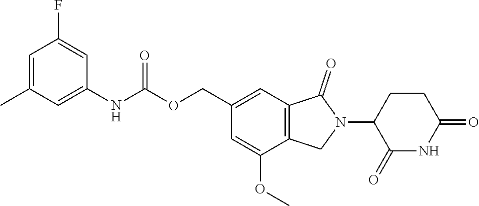 [SMILES: COc1cc(COC(=O)Nc2cc(C)cc(F)c2)cc2c1CN(C1CCC(=O)NC1=O)C2=O] | 567 |
| 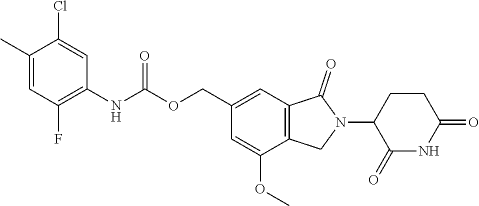 [SMILES: COc1cc(COC(=O)Nc2cc(Cl)c(C)cc2F)cc2c1CN(C1CCC(=O)NC1=O)C2=O] | 568 |
| 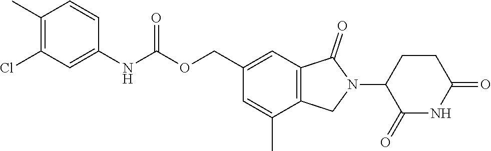 [SMILES: Cc1ccc(NC(=O)OCc2cc(C)c3c(c2)C(=O)N(C2CCC(=O)NC2=O)C3)cc1Cl] | 569 |
| 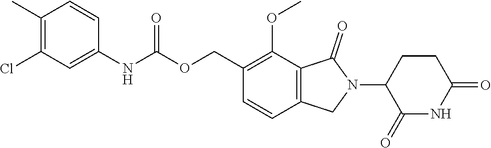 [SMILES: COc1c(COC(=O)Nc2ccc(C)c(Cl)c2)ccc2c1C(=O)N(C1CCC(=O)NC1=O)C2] | 570 |
| 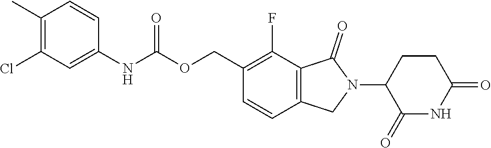 [SMILES: Cc1ccc(NC(=O)OCc2ccc3c(c2F)C(=O)N(C2CCC(=O)NC2=O)C3)cc1Cl] | 571 |
| 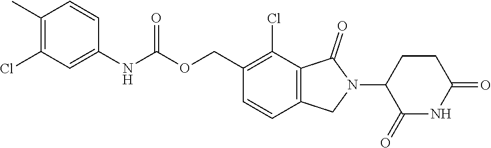 [SMILES: Cc1ccc(NC(=O)OCc2ccc3c(c2Cl)C(=O)N(C2CCC(=O)NC2=O)C3)cc1Cl] | 572 |
| 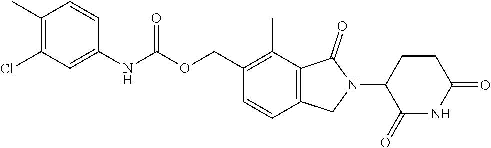 [SMILES: Cc1ccc(NC(=O)OCc2ccc3c(c2C)C(=O)N(C2CCC(=O)NC2=O)C3)cc1Cl] | 573 |

Compounds 501-573 can be prepared using variations of the methods used to prepare compounds 201-440. These variations are known to those of skill in the art.

### Example 2: IC50 Data—Compound Activity by Fluorescent Polarization Assay

Compound activity was monitored in a fluorescence polarization (FP) homogeneous assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Unless otherwise stated, all reagents were purchased from Sigma Aldrich. Enzymatic reactions were conducted in Perkin-Elmer Black 384 well ProxiPlate Plus (catalogue no. 6008269) in 10 μL total volume. Full length wild-type cereblon CRBN (80.0 nM, 10 μL) was incubated in assay buffer containing 20 mM HEPES (pH 8.0), 150 NaCl, 0.5 mM TCEP and 0.05% Tween 20 in the presence or absence of compound (300 nL). Inhibitors were stored as 10 mM DMSO stocks in an inert environment (low humidity, dark, low oxygen, room temperature) using the Storage Pod System. Compounds and DMSO were dispensed using the Echo E5XX (Labcyte Inc. USA) to give concentrations from 300 to 0.937 or 3000 to 9.3 nM in a 12 data point curve. Mutant YWAA CRBN (80.0 nM, 10 μL) which does not interact with the fluorescent probe was used as a negative control for the assay. Following incubation at room temperature for 30 min, the assay was initiated by dispensing the probe to a final concentration of 5 nM (2.5 nL of a 20 μM stock) using the Echo E5XX. FP was measured after a period of 12 hours using a Pherastar plate reader (BMG Labtech, Germany) exciting at 590 nm and measuring the amount of parallel and perpendicular light at 675 nm. The FP signal was subsequently normalized to the no-compound control (i.e., DMSO). Analysis and IC50 values were derived using Dotmatics (Dotmatics UK) software.

TABLE 3a
IC50 values of compounds 1 to 165 and 201 to 307
determined in the fluorescence polarization
assay indicating the cereblon binding

| | rFP IC50 |
|---|---|
| Compound | [nM] |
| 1 | B |
| 2 | A |
| 3 | A |
| 4 | A |
| 5 | A |
| 6 | A |
| 7 | A |
| 8 | A |
| 9 | B |
| 10 | B |
| 11 | C |
| 12 | B |
| 13 | B |
| 14 | C |
| 15 | B |
| 16 | B |
| 17 | B |
| 18 | B |
| 19 | B |
| 20 | B |
| 21 | B |
| 22 | A |
| 23 | A |
| 24 | C |
| 25 | B |
| 26 | B |
| 27 | A |
| 28 | B |
| 29 | C |
| 30 | B |
| 31 | B |
| 32 | B |
| 33 | B |
| 34 | A |
| 35 | B |
| 36 | B |
| 37 | B |
| 38 | B |
| 39 | B |
| 40 | B |
| 41 | B |
| 42 | B |
| 43 | B |
| 44 | B |
| 45 | A |
| 46 | C |
| 47 | B |
| 48 | B |
| 49 | B |
| 50 | A |
| 51 | A |
| 52 | A |
| 53 | A |
| 54 | A |
| 55 | A |
| 56 | A |
| 57 | A |
| 58 | B |
| 59 | A |
| 60 | A |
| 61 | A |
| 62 | A |
| 63 | A |
| 64 | A |
| 65 | B |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | B |
| 78 | B |
| 79 | A |
| 80 | B |
| 81 | B |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | B |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | B |
| 123 | B |
| 124 | A |
| 125 | B |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | C |
| 138 | B |
| 139 | B |
| 140 | B |
| 141 | A |
| 142 | B |
| 143 | D |
| 144 | B |
| 145 | B |
| 146 | A |
| 147 | A |
| 148 | A |
| 149 | C |
| 150 | A |
| 151 | B |
| 152 | B |
| 153 | D |
| 154 | B |
| 155 | B |
| 156 | B |
| 157 | C |
| 158 | C |
| 159 | B |
| 160 | A |
| 161 | A |
| 162 | B |
| 163 | C |
| 164 | C |
| 165 | C |
| 201 | C |
| 202 | B |
| 203 | C |
| 204 | B |
| 205 | C |
| 206 | B |
| 207 | B |
| 208 | B |
| 209 | C |
| 210 | C |
| 211 | D |
| 212 | C |
| 213 | B |
| 214 | C |
| 215 | B |
| 216 | A |
| 217 | D |
| 218 | B |
| 219 | D |
| 220 | C |
| 221 | D |
| 222 | D |
| 223 | A |
| 224 | B |
| 225 | B |
| 226 | B |
| 227 | D |
| 228 | C |
| 229 | B |
| 230 | A |
| 231 | D |
| 232 | D |
| 233 | B |
| 234 | D |
| 235 | A |
| 236 | A |
| 237 | D |
| 238 | B |
| 239 | B |
| 240 | A |
| 241 | B |
| 242 | D |
| 243 | D |
| 244 | B |
| 245 | D |
| 246 | A |
| 247 | C |
| 248 | C |
| 249 | C |
| 250 | D |
| 251 | B |
| 252 | B |
| 253 | B |
| 254 | A |
| 255 | B |
| 256 | C |
| 257 | D |
| 258 | B |
| 259 | A |
| 260 | A |
| 261 | D |
| 262 | B |
| 263 | A |
| 264 | B |
| 265 | A |
| 266 | A |
| 267 | B |
| 268 | B |
| 269 | A |
| 270 | A |
| 271 | C |
| 272 | B |
| 273 | A |
| 274 | A |
| 275 | A |
| 276 | B |
| 277 | B |
| 278 | A |
| 279 | C |
| 280 | A |
| 281 | A |
| 282 | A |
| 283 | B |
| 284 | A |
| 285 | D |
| 286 | B |
| 287 | B |
| 288 | B |
| 289 | B |
| 290 | C |
| 291 | B |
| 292 | B |
| 293 | B |
| 294 | A |
| 295 | C |
| 296 | C |
| 297 | B |
| 298 | B |
| 299 | C |
| 300 | B |
| 301 | D |
| 302 | B |
| 303 | B |
| 304 | D |
| 305 | B |
| 306 | B |
| 307 | A |

In some embodiments, the disclosure is directed to compounds with an IC50 value of less than 1100 nM, i.e. directed to compounds 1, 2, 3, 4, 5, 6, 7, 8, 9, 10, 12, 13, 15, 16, 17, 18, 19, 20, 21, 22, 23, 26, 26, 27, 28, 30, 31, 32, 33, 34, 35, 36, 37, 38, 39, 40, 41, 42, 43, 44, 45, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131,132, 133, 134, 135, 136, 138, 139, 140, 141, 142, 144, 145, 146, 147, 148, 150, 151, 152, 154, 155, 156, 158, 159, 160, 161,162, 202, 204, 206, 207, 208, 213, 215, 216, 218, 223, 224, 225, 226, 229, 230, 233, 235, 236, 238, 239, 240, 241, 244, 246, 251, 252, 253, 254, 255, 258, 259, 260, 262, 263, 264, 265, 266, 267, 268, 269, 270, 272, 273, 274, 275, 276, 277, 278, 280, 281, 282, 283, 284, 286, 287, 288, 289, 291, 292, 293, 294, 297, 298, 300, 302, 303, 305, 306, and 307.

TABLE 3B
IC50 values determined in the fluorescence polarization
assay indicating the cereblon binding

| | rFP IC50 |
|---|---|
| Compound | [nM] |
| 501 | D |
| 502 | D |
| 503 | D |
| 504 | D |
| 505 | B |
| 506 | D |
| 507 | C |
| 508 | D |
| 509 | C |
| 510 | D |
| 511 | A |
| 512 | D |
| 513 | D |
| 514 | D |
| 515 | D |
| 516 | B |
| 517 | A |
| 518 | C |
| 519 | A |
| 520 | B |
| 521 | C |
| 522 | D |
| 523 | B |
| 524 | D |
| 525 | D |
| 526 | C |
| 527 | C |
| 528 | D |
| 529 | D |
| 530 | D |
| 531 | D |
| 532 | D |
| 533 | D |
| 534 | D |
| 535 | D |
| 536 | C |
| 537 | A |
| 538 | A |
| 539 | C |
| 540 | A |
| 541 | C |
| 542 | B |
| 543 | B |
| 544 | A |
| 545 | B |
| 546 | B |
| 547 | B |
| 548 | C |
| 549 | B |
| 550 | B |
| 551 | B |
| 552 | A |
| 553 | A |
| 554 | A |
| 555 | B |
| 556 | A |
| 557 | A |
| 558 | A |
| 559 | A |
| 560 | A |
| 561 | D |
| 562 | A |
| 563 | |
| 564 | |
| 565 | |
| 566 | |
| 567 | |
| 568 | |
| 569 | — |
| 570 | D |
| 571 | D |
| 572 | D |
| 573 | A |

Table 3B assigns each compound a code indicating the ability for cereblon binding by means of their IC50 values: A, B, C or D. According to the code, A represents an IC50 value of ≤600 nM, B represents an IC50 value>600 nM and ≤1200 nM, C represents an IC50 value of >1200 nM and ≤1900 nM and D represents an IC50 value of >1900 nM.

In some embodiments, the disclosure is directed to compounds with an IC50 value of less than 1900 nM, i.e. directed to compounds 11, 17, 19, 37, 38, 40, 44, 52, 53, 54, 56, 57, 58, 59, 60, and 62.

In some embodiments, the disclosure is directed to compounds with an IC50 value of less than 1200 nM, i.e. directed to compounds 5, 11, 16, 17, 19, 20, 23, 37, 38, 40, 42, 43, 44, 45, 46, 47, 49, 50, 51, 52, 53, 54, 56, 57, 58, 59, 60, and 62.

In some embodiments, the disclosure is directed to compounds with an IC50 value of less than 600 nM, i.e. directed to compounds 5, 7, 9, 11, 16, 17, 18, 19, 20, 21, 23, 26, 27, 36, 37, 38, 39, 40, 41, 42, 43, 44, 45, 46, 47, 48, 49, 50, 51, 52, 53, 54, 56, 57, 58, 59, 60, and 62.

### Example 3: Compound Binding by Immunofluorescence Assay

In order to demonstrate the ability of the compounds to bind to degrade a specific protein of interest, GSPT1 was chosen and tested in an immunofluorescence assay. CAL-51 cells were purchased from DSMZ (cat. Number ACC302), sub-cultured in 90% Dulbecco's MEM (4.5 g/L glucose, Gibco 11965)+10% heat inactivated FBS (BioConcept, 2-01F1361) and incubated at 37° C., 5% CO₂. For the assay, imaging microtiterplate Cell Carrier 96 Ultra (Perkin Elmer 6055302) were pre-coated with Fibronectin (Sigma F085, 30 μl at 0.2 μg/ml) in PBS (100 μl, Gibco 14190) for 45 min at room temperature, rinsed with PBS and CAL-51 cells (30K cells/well) were plated and let to adhere overnight. Cells were treated with compounds typically using a serial dilution ranging from 30 μM to 0.1 nM for 6 hours. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50 μl, Sigma HT5011)) for 20 mins at room temperature. Following three consecutive PBS washes (100 μl), cells were permeabilized in 0.1% Triton X-100 in PBS (Sigma 93443, 50 μl) for 15 mins at room temperature. Following three further PBS washes, 50 μl blocking buffer (1% BSA, Sigma A4503, in PBS) was added for 45 min for signal-to-noise reduction. Primary antibody (human GSPT1, Sigma HPA052488) was diluted in blocking buffer (dil. 1/300, 35 μl/well) and incubated with the cells overnight at 4° C. After three PBS washes, Alexa-fluor 488 coupled secondary antibodies (Invitrogen, A32731, dil. 1/1000), Alexa-fluor 647-Phalloidin (Invitrogen, A22287, dil. 1/200) and DAPI (Thermo, #62248, dil. 1/1000) were diluted in blocking buffer and incubated with the samples for 2 hours at room temperature. After three final PBS washes, samples were conserved in 100 μl PBS in the dark, until measurement. Image acquisition was performed on the Operetta High-Content Imager (Perkin-Elmer). Fluorescence intensity of Alexa-Fluor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus. The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

Table 4 assigns each compound a code indicating the ability for GSPT1 degradation: A, B or C. According to the code, A represents a DC50 value of ≤100 nM, B represents a DC50 value>100 nM and ≤300 nM and C represents a DC50 value of >300 nM.

TABLE 4
Activity for GSPT1 degradation for compounds 1 to 165

| Compound | Code |
|---|---|
| 1 | C |
| 2 | C |
| 3 | C |
| 4 | C |
| 5 | C |
| 6 | C |
| 7 | C |
| 8 | A |
| 9 | C |
| 10 | C |
| 11 | C |
| 12 | C |
| 13 | C |
| 14 | C |
| 15 | C |
| 16 | C |
| 17 | C |
| 18 | C |
| 19 | C |
| 20 | A |
| 21 | C |
| 22 | C |
| 23 | C |
| 24 | C |
| 25 | C |
| 26 | C |
| 27 | C |
| 28 | C |
| 29 | C |
| 30 | A |
| 31 | B |
| 32 | C |
| 33 | A |
| 34 | C |
| 35 | C |
| 36 | C |
| 37 | C |
| 38 | B |
| 39 | C |
| 40 | C |
| 41 | B |
| 42 | B |
| 43 | C |
| 44 | C |
| 45 | C |
| 46 | A |
| 47 | A |
| 48 | C |
| 49 | C |
| 50 | C |
| 51 | C |
| 52 | C |
| 53 | C |
| 54 | C |
| 55 | C |
| 56 | A |
| 57 | C |
| 58 | C |
| 59 | B |
| 60 | C |
| 61 | A |
| 62 | C |
| 63 | C |
| 64 | C |
| 65 | C |
| 66 | C |
| 67 | C |
| 68 | B |
| 69 | C |
| 70 | C |
| 71 | A |
| 72 | C |
| 73 | C |
| 74 | C |
| 75 | B |
| 76 | A |
| 77 | A |
| 78 | A |
| 79 | C |
| 80 | C |
| 81 | C |
| 82 | A |
| 83 | C |
| 84 | C |
| 85 | B |
| 86 | C |
| 87 | C |
| 88 | C |
| 89 | B |
| 90 | A |
| 91 | C |
| 92 | C |
| 93 | C |
| 94 | C |
| 95 | C |
| 96 | C |
| 97 | C |
| 98 | C |
| 99 | C |
| 100 | C |
| 101 | B |
| 102 | C |
| 103 | C |
| 104 | C |
| 105 | C |
| 106 | B |
| 107 | C |
| 108 | C |
| 109 | B |
| 110 | C |
| 111 | C |
| 112 | C |
| 113 | C |
| 114 | C |
| 115 | B |
| 116 | C |
| 117 | C |
| 118 | A |
| 119 | A |
| 120 | C |
| 121 | B |
| 122 | A |
| 123 | C |
| 124 | C |
| 125 | A |
| 126 | C |
| 127 | A |
| 128 | C |
| 129 | A |
| 130 | C |
| 131 | C |
| 132 | A |
| 133 | C |
| 134 | C |
| 135 | C |
| 136 | C |
| 137 | C |
| 138 | A |
| 139 | A |
| 140 | A |
| 141 | A |
| 142 | A |
| 143 | B |
| 144 | A |
| 145 | A |
| 146 | C |
| 147 | C |
| 148 | C |
| 149 | C |
| 150 | C |
| 151 | A |
| 152 | C |
| 153 | C |
| 154 | C |
| 155 | B |
| 156 | C |
| 157 | C |
| 158 | C |
| 159 | C |
| 160 | A |
| 161 | C |
| 162 | C |
| 163 | A |
| 164 | C |
| 165 | C |

Table 5A assigns each compound a code indicating the ability for GSPT1 degradation: A, B or C. According to the code, A represents a DC50 value of ≤30 nM, B represents a DC50 value>30 nM and ≤300 nM and C represents a DC50 value of >300 nM.

TABLE 5A
Activity for GSPT1 degradation for compounds 201 to 307

| Compound | Code |
|---|---|
| 201 | A |
| 202 | A |
| 203 | A |
| 204 | A |
| 205 | A |
| 206 | A |
| 207 | B |
| 208 | A |
| 209 | B |
| 210 | A |
| 211 | A |
| 212 | B |
| 213 | A |
| 214 | C |
| 215 | A |
| 216 | C |
| 217 | C |
| 218 | B |
| 219 | B |
| 220 | C |
| 221 | A |
| 222 | C |
| 223 | C |
| 224 | A |
| 225 | A |
| 226 | C |
| 227 | C |
| 228 | C |
| 229 | C |
| 230 | C |
| 231 | B |
| 232 | B |
| 233 | B |
| 234 | A |
| 235 | C |
| 236 | C |
| 237 | B |
| 238 | C |
| 239 | A |
| 240 | B |
| 241 | A |
| 242 | A |
| 243 | A |
| 244 | B |
| 245 | B |
| 246 | A |
| 247 | A |
| 248 | A |
| 249 | A |
| 250 | A |
| 251 | B |
| 252 | C |
| 253 | B |
| 254 | C |
| 255 | A |
| 256 | B |
| 257 | A |
| 258 | A |
| 259 | B |
| 260 | B |
| 261 | A |
| 262 | B |
| 263 | C |
| 264 | B |
| 265 | C |
| 266 | B |
| 267 | B |
| 268 | A |
| 269 | A |
| 270 | B |
| 271 | B |
| 272 | B |
| 273 | B |
| 274 | A |
| 275 | B |
| 276 | B |
| 277 | A |
| 278 | B |
| 279 | A |
| 280 | B |
| 281 | B |
| 282 | C |
| 283 | A |
| 284 | A |
| 285 | A |
| 286 | A |
| 287 | A |
| 288 | A |
| 289 | C |
| 290 | A |
| 291 | A |
| 292 | A |
| 293 | B |
| 294 | B |
| 295 | A |
| 296 | A |
| 297 | C |
| 298 | C |
| 299 | B |
| 300 | B |
| 301 | B |
| 302 | C |
| 303 | B |
| 304 | B |
| 305 | B |
| 306 | B |
| 307 | C |

TABLE 5B
Activity for GSPT1 degradation

| Compound | Code |
|---|---|
| 501 | B |
| 502 | B |
| 503 | B |
| 504 | B |
| 505 | A |
| 506 | B |
| 507 | B |
| 508 | A |
| 509 | A |
| 510 | B |
| 511 | A |
| 512 | B |
| 513 | B |
| 514 | B |
| 515 | B |
| 516 | A |
| 517 | A |
| 518 | A |
| 519 | A |
| 520 | A |
| 521 | A |
| 522 | A |
| 523 | A |
| 524 | B |
| 525 | A |
| 526 | B |
| 527 | B |
| 528 | B |
| 529 | A |
| 530 | B |
| 531 | B |
| 532 | B |
| 533 | B |
| 534 | B |
| 535 | A |
| 536 | A |
| 537 | A |
| 538 | A |
| 539 | A |
| 540 | A |
| 541 | B |
| 542 | A |
| 543 | A |
| 544 | A |
| 545 | A |
| 546 | B |
| 547 | A |
| 548 | B |
| 549 | A |
| 550 | A |
| 551 | B |
| 552 | B |
| 553 | A |
| 554 | A |
| 555 | A |
| 556 | A |
| 557 | A |
| 558 | C |
| 559 | A |
| 560 | A |
| 561 | A |
| 562 | B |
| 563 | B |
| 564 | B |
| 565 | B |
| 566 | C |
| 567 | A |
| 568 | C |
| 570 | D |
| 571 | A |
| 572 | D |
| 573 | D |

Table 5B assigns each compound a code indicating the ability for GSPT1 degradation: A, B or C. According to the code, A represents a DC50 value of ≤30 nM, B represents a DC50 value>30 nM and ≤300 nM and C represents a DC50 value of >300 nM.

In some embodiments, the compounds of any of formula I to XIII exhibits a DC50 value of 30 nM or less, i.e. compounds with code A. In some embodiments the compound is selected from the group consisting of 5, 8, 9, 11, 16, 17, 18, 19, 20, 21, 22, 23, 25, 29, 35, 36, 37, 38, 39, 40, 42, 43, 44, 45, 47, 49, 50, 53, 54, 55, 56, 57, 59, 60, 61, 67 and 71.

### Example 4: 3D Spheroid Experiments—HMEC

Human mammary epithelial cells (HMEC) were engineered to express either c-Myc tagged with EGFP or EGFP alone (non myc) (analogous but distinct from Kessler J D et al, Science. 2012 Jan. 20; 335(6066):348-53. doi: 10.1126/science.1212728; Hsu T Y et al, Nature. 2015 Sep. 17; 525(7569):384-8. doi: 10.1038/nature14985). The engineered cells were seeded at 1000 (myc) or 4000 (non myc) cells/well in a 384 ultra-low attachment plate in a total volume of 40 μL HMEC cell culture media (DMEM/F12+10% HI-FBS+15 mM HEPES+0.5 ug/ml Hydrocortisone+10 ug/ml Insulin+20 ng/ml EGF). Plates were spun at 1200 rpm for 5 minutes at room temperature to ensure that cells have gathered in the middle of the well and incubated at 37° C. for 48 hrs before firing with compounds. On Day 2 (48 h post seeding) cells were imaged (brightfield and EGFP fluorescence) using the Celigo imaging cytometer prior to firing compounds. For primary screens, compounds were added at three concentrations (1.25, 10 and 30 uM) in a volume of 120 nL using the ECHO acoustic dispenser and spun at 2000 rpm for 2 minutes at room temperature before incubating at 37° C. for 5 days. For counter-screen and establishment of IC50 concentrations, a 12-point dose was prepared starting from 30 uM with 3-fold dilutions and added to cells in a volume of 120 nL using the ECHO acoustic dispenser and plates spun at 2000 rpm for 2 minutes at room temperature before incubating at 37° C. for 5 days. On Day 7, prior to measuring cell viability using CellTiterGlo3D, cells were imaged (brightfield and EGFP fluorescence) on the Celigo imaging cytometer. The CellTiterGlo3D reagent is added at 30 uL/well and incubated at room temperature for 30 mins. After 30 mins of incubation, luminescence readings were recorded using the Perkin Elmer EnVision reader. Table 6 assigns each compound a code indicating the EC50 value in the myc-HMEC assay as well as in the non myc-HMEC assay: D, E, F or G. According to the code, D represents an EC50 value of ≤400 nM in the myc-HMEC assay, E represents an EC50 value>400 nM and ≤2000 nM in the myc-HMEC assay, F represents an EC50 value of >2000 nM in the myc-HMEC assay and G represents an EC50 value of >10000 nM in the non myc-HMEC assay.

TABLE 6
3D spheroid assay

| Com- | | | Com- | | | Com- | | |
|---|---|---|---|---|---|---|---|---|
| pound | Code | Code | pound | Code | Code | pound | Code | Code |
| 1 | F | G | 2 | F | G | 3 | F | G |
| 4 | F | G | 5 | F | G | 6 | F | G |
| 7 | F | G | 8 | D | G | 9 | F | G |
| 10 | F | G | 11 | F | G | 12 | F | G |
| 13 | F | G | 14 | F | G | 15 | F | G |
| 16 | F | G | 17 | F | G | 18 | F | G |
| 19 | F | G | 20 | E | G | 21 | F | G |
| 22 | F | G | 23 | F | G | 24 | F | G |
| 25 | F | G | 26 | F | G | 27 | F | G |
| 28 | F | G | 29 | F | G | 30 | D | G |
| 31 | E | G | 32 | F | G | 33 | D | G |
| 34 | F | G | 35 | F | G | 36 | F | G |
| 37 | F | G | 38 | D | G | 39 | F | G |
| 40 | F | G | 41 | E | G | 42 | E | G |
| 43 | F | G | 44 | F | G | 45 | F | G |
| 46 | E | G | 47 | E | G | 48 | F | G |
| 49 | F | G | 50 | F | G | 51 | F | G |
| 52 | F | G | 53 | F | G | 54 | F | G |
| 55 | F | G | 56 | D | G | 57 | F | G |
| 58 | F | G | 59 | E | G | 60 | F | G |
| 61 | D | G | 62 | F | G | 63 | F | G |
| 64 | F | G | 65 | F | G | 66 | F | G |
| 67 | F | G | 68 | E | G | 69 | F | G |
| 70 | F | G | 71 | D | G | 72 | F | G |
| 73 | F | G | 74 | F | G | 75 | | G |
| 76 | | G | 77 | | G | 78 | | G |

In some embodiments, the compounds of any of formula I to IV exhibits an EC50 value of 2000 nM or less, i.e. compounds with code D and E. In some embodiments the compound is selected from the group consisting of 8, 20, 30, 31, 33, 38, 41, 42, 46, 47, 56, 59, 61, 68, and 71.

In some embodiments, the compounds of any of formula I to IV exhibits an EC50 value of 400 nM or less, i.e. compounds with code D. In some embodiments the compound is selected from the group consisting of 8, 30, 33, 38, 56, 61 and 71.

### Example 5: Viability Assay

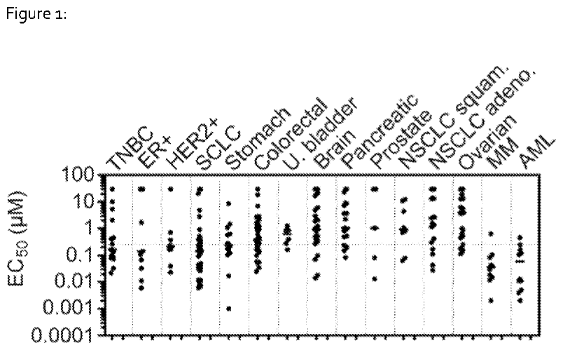

Compound 8 was tested on a panel of 303 cell lines using a 3-day CellTiter Glo assay format. Cells were sub-cultures as recommended by the providers (ATCC, DSMZ) and incubated at 37° C., 5% CO2. All cells were authenticated and confirmed by Short Tandem Repeat (STR) profiling. Briefly, cells were seeded to the appropriate density to allow logarithmic growth over the assay period in 96-well opaque-walled clear bottom plates (from Corning, Cat. No. 3903, Lot. No. 16419061; 100 μL total volume) and incubated overnight. Compound 8 and DMSO control were subsequently dispensed to the plates using HPD300 and a 9 data-point titration curve with concentrations ranging from 30 μM to 0.1 nM. Dilutions were prepared fresh prior to the assay using a 10 mM DMSO stock. Final DMSO concentration was 0.5%. The plates were incubated for 3 days at 5% CO2, 37° C. Cell viability was assessed using the CellTiter-Glo assay kit as recommended by the Manufacturer (Promega, Cat. No. G7558, Lot. No. 0000385738) and measurements performed on the EnSpire multimode plate reader (Perkin Elmer). EC50 values were determined using Prism8 (GraphPad) and the asymmetric (five-parameter) equation and least squares fit (see FIG. 1).

### Example 6: Pharmacogenomics and Clustering Analyses

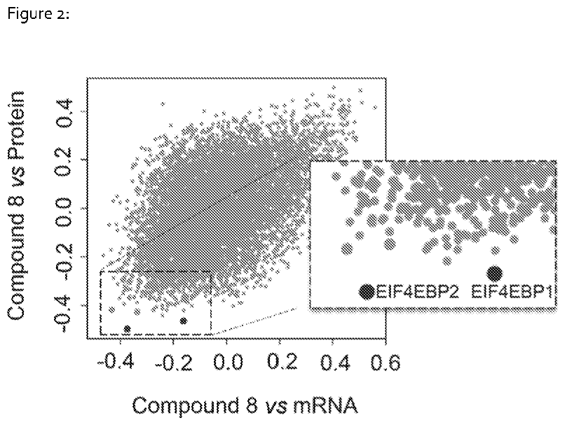

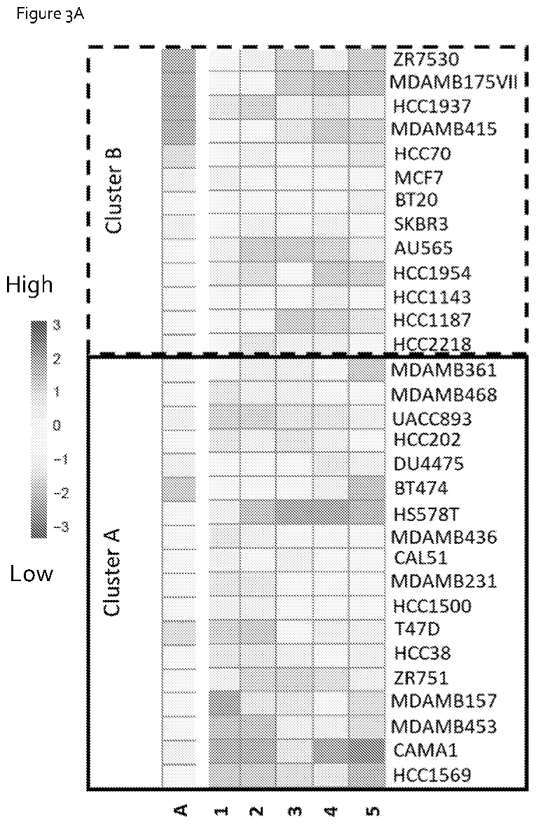

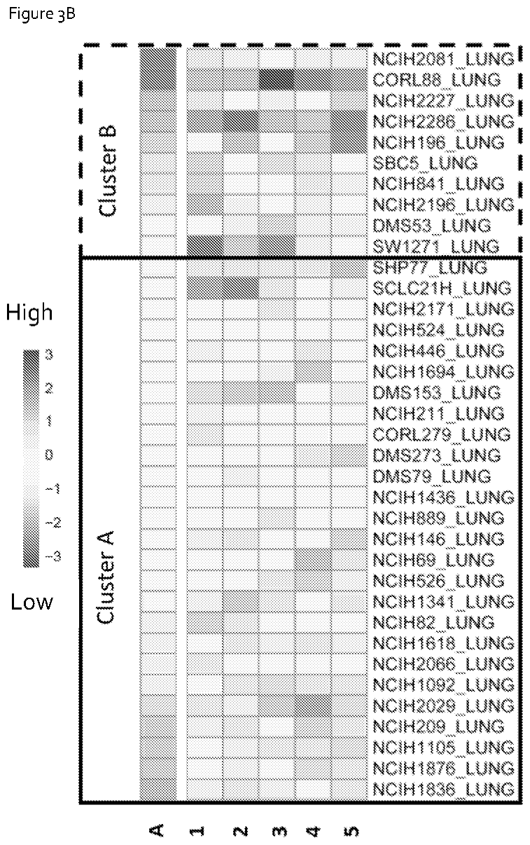

For each gene, Pearson correlation coefficients were calculated between compound 8 sensitivity values (EC50s) and protein expression levels and between compound 8 sensitivity values (EC50s) and mRNA expression levels in the corresponding cell lines. Protein expression and mRNA expression data were obtained from the Cancer Cell Lines Encyclopedia (CCLE) project (Ghandi et al. Nature 2019, 569, 503-508; Nusinow et al. Cell 2019, 180, 387-402). Genes with protein expression level quantified in fewer than 100 cell lines were excluded. The Pearson correlation coefficients for compound 8 sensitivity vs mRNA expression and compound 8 sensitivity vs protein expression were plotted using a scatter plot and genes with outlier correlation with compound 8 sensitivity (EIF4EBP1, EIF4EBP2) were identified (see FIG. 2). The mRNA expression, protein and phospho protein data from the CCLE RNAseq and Reverse-phase protein arrays (RPPA) datasets for these genes were then applied to unsupervised hierarchical clustering analysis for each cancer type. This was done using pheatmap function in R programming language with the clustering method parameter set to “ward.D”. Hierarchical clustering is an unsupervised clustering method assigns the samples with higher similarity in the feature space to similar clusters. By applying this method to the breast cancer cell lines data, a cluster with high EIF4EBP1/EIF4EBP2 that corresponded with higher sensitivity to compound 8 and a cluster with low EIF4EBP1/EIF4EBP2 that corresponded with lower sensitivity to compound 8 was identified. Therefore, concluding that high EIF4EBP1/EIF4EBP2 is a marker of sensitivity to compound 8 in breast cancer and could be used to predict the treatment efficacy.

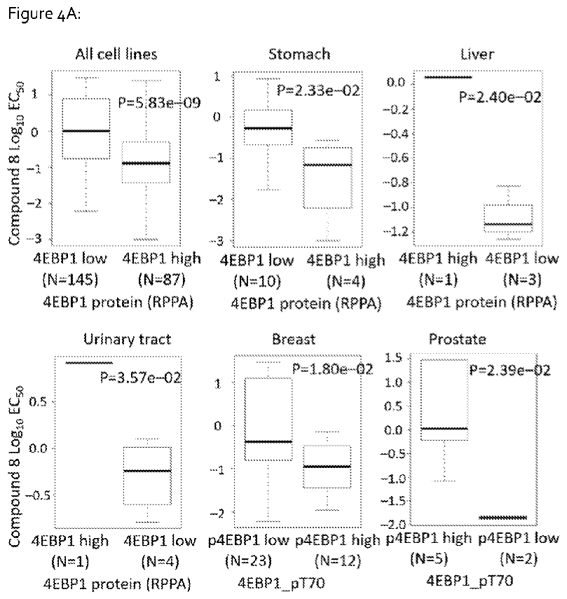

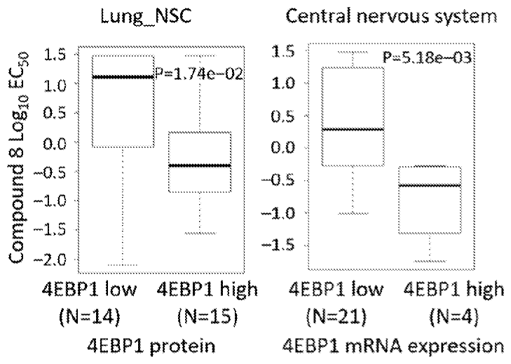

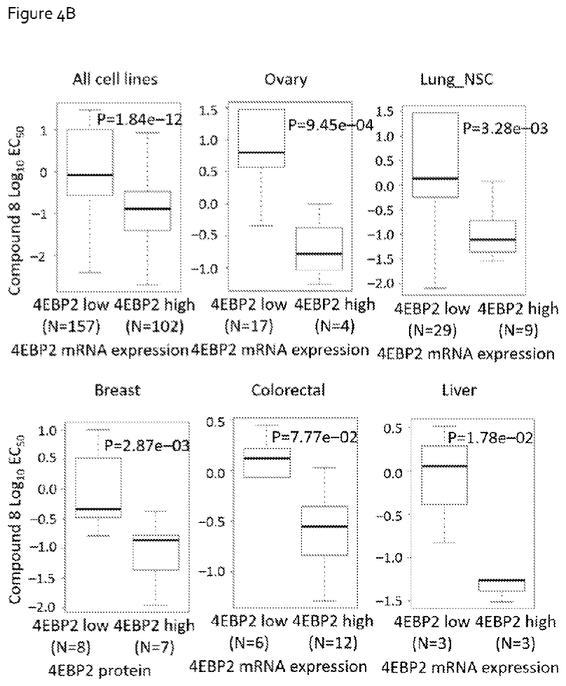

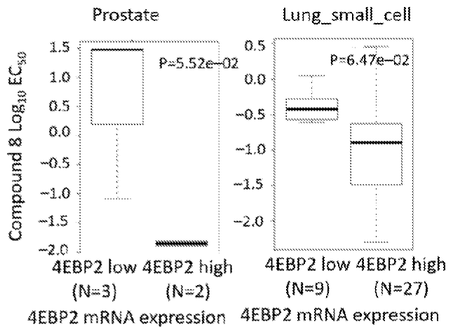

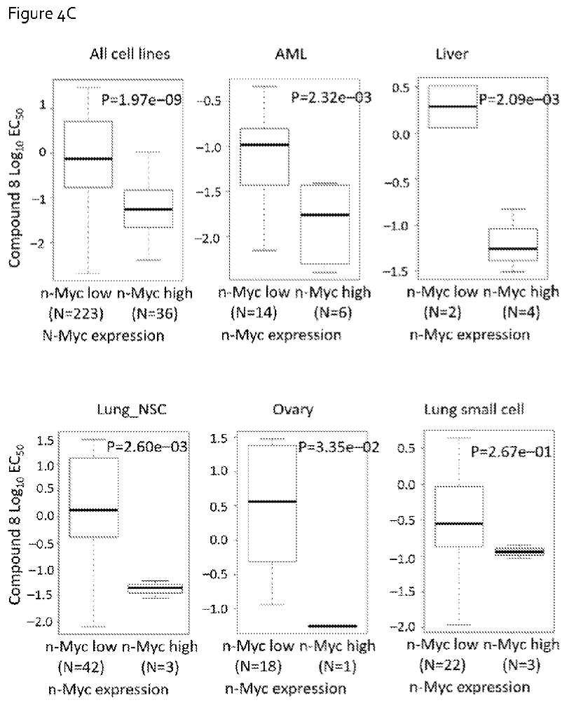

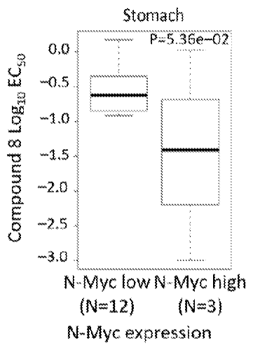

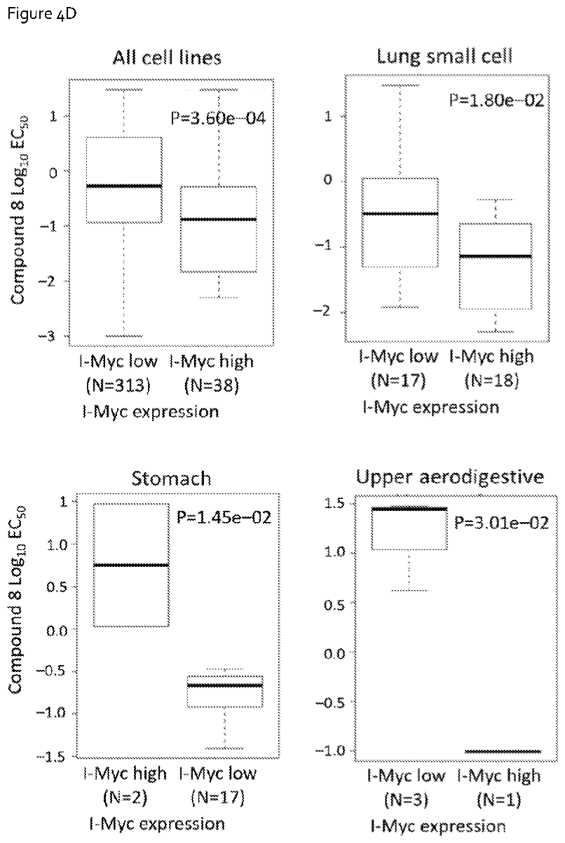

Based on an evaluation of the association between Myc signaling related genes, specifically C-Myc, N-Myc, and L-Myc mRNA expression and sensitivity to compound 8 using Pearson correlation analysis, cancer types with significant correlation between N-Myc and L-Myc expression and sensitivity to compound 8 were identified. In particular, high N-Myc and L-Myc expression level, respectively, to be correlated with sensitivity to compound 8 in NSCLC, stomach cancer and acute myeloid leukemia (AML) and SCLC, respectively (see FIGS. 4C and 4D), was identified.

### Example 7

Example 7A: Representative associations between 4EBP1 levels and sensitivity to GSPT1 degrader compound 8. High 4EBP1 levels is associated with the highest levels of sensitivity to compound 8. Representative examples of cancer subtypes include, but are not restricted to, prostate, breast, stomach, liver, central nervous system, urinary track, non small cell lung cancers (see FIG. 4A).

Example 7B: Representative associations between 4EBP2 levels and sensitivity to GSPT1 degrader Compound 8. High 4EBP2 levels is associated with the highest levels of sensitivity to Compound 8. Representative examples of cancer subtypes include, but are not restricted to, liver, non small cell lung, small cell lung, breast, prostate, ovary cancers (see FIG. 4B).

Example 7C: Representative associations between N-Myc levels and sensitivity to GSPT1 degrader Compound 8. High N-Myc levels is associated with the highest levels of sensitivity to Compound 8. Representative examples of cancer subtypes include, but are not restricted to, AML, liver, non small cell lung, small cell lung, ovary, stomach cancers (see FIG. 4C).

Example 7D: Representative associations between L-Myc levels and sensitivity to GSPT1 degrader Compound 8. High L-Myc levels is associated with the highest levels of sensitivity to Compound 8. Representative examples of cancer subtypes include, but are not restricted to, small cell lung, upper digestive, stomach cancers (see FIG. 4D).

### Example 8: C-Myc and CRBN Dependency on the C-Myc HMEC Isogenic Cell Line Model

Human mammary epithelial cells (HMEC) were engineered to express either c-Myc tagged with EGFP or EGFP alone (non myc) (analogous but distinct from Kessler J D et al, Science. 2012 Jan. 20; 335(6066):348-53. doi: 10.1126/science.1212728; Hsu T Y et al, Nature. 2015 Sep. 17; 525(7569):384-8. doi: 10.1038/nature14985). The engineered cells were plated in 6 well plates or seeded at 1000 (myc) or 4000 (non myc) cells/well in a 384 ultra-low attachment plate in a total volume of 40 μL HMEC cell culture media (DMEM/F12+10% HI-FBS+15 mM HEPES+0.5 ug/ml Hydrocortisone+10 ug/ml Insulin+20 ng/ml EGF). Cells were cultured in the presence or absence of doxycycline for 25 population doublings and cell lysates generated for western blotting probing for c-Myc, 4EBP1, phosphor-4EBP1 (p4EBP1) as indicated. β-actin used as a loading control. For the wash-off lane, doxycycline was removed 24 hours before cell lysis. Cell viability was assessed in the presence (Myc on) or absence (Myc off, −Dox) of doxycycline and also following doxycycline removal (Myc off, wash-off). Cell viability was performed following treatment with compound 8 for 72 hours as recommended by the provider (Promega, CellTiterGlo). Similarly, CRBN-dependency was assessed in the HMEC proficient and deficient cell lines induced for c-Myc expression with doxycycline and viability performed as above following 72 hours of treatment with compound 8. EC50 values were determined using Prism8 (GraphPad) and the asymmetric (five-parameter) equation and least squares fit.

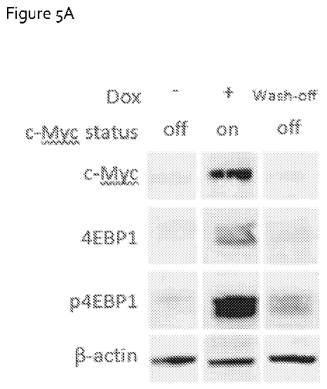

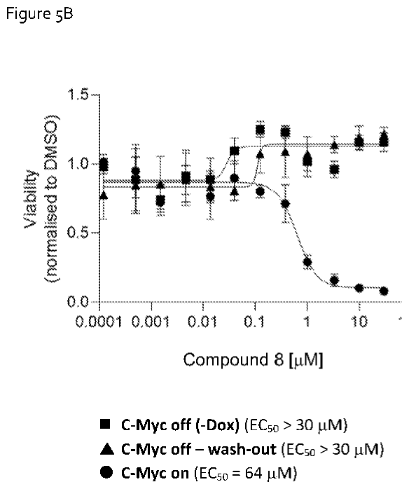

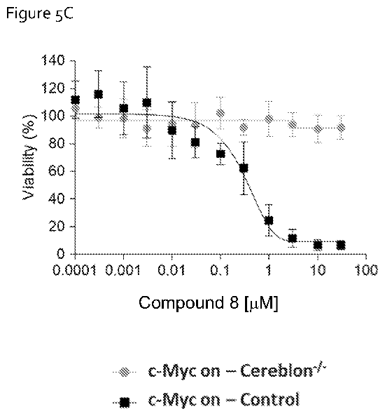

FIG. 5A shows that after c-Myc induction, the cells displayed key biomarkers of enhanced protein translation, including upregulation and phosphorylation of 4EBP1. FIG. 5B shows that compound 8 induced cell death with an EC50 of 0.64 μM in the presence of high c-Myc expression but did not induce cell death at the highest concentration tested of 30 μM in the absence of doxycycline-driven c-Myc expression or after doxycycline was washed out to remove c-Myc expression in cells that previously expressed c-Myc. FIG. 5C shows that compound 8 did not induce death in cells for which cereblon was knocked out, confirming cereblon-dependence of compound 8's viability effect.

### Example 9: Viability Assay and GSPT1 Degradation in Representative NSCLC Cell Lines

The anti-proliferative activity of compound 210 was tested on representative N-Myc high and N-Myc low cell lines (NCI-H1155, ABC-1 and EBC-1, NCI-H2023, respectively) following treatment with compound for 72 hours. Cells were sub-cultures as recommended by the providers (ATCC, DSMZ) and incubated at 37° C., 5% CO2. All cells were authenticated and confirmed by Short Tandem Repeat (STR) profiling. Briefly, cells were seeded to the appropriate density to allow logarithmic growth over the assay period in 96-well opaque-walled clear bottom plates (from Corning, Cat. No. 3903, Lot. No. 16419061; 100 μL total volume) and incubated overnight. Compound 210 and DMSO control were subsequently dispensed to the plates using HPD300 and a 9 data-point titration curve with concentrations ranging from 30 μM to 0.1 nM. Dilutions were prepared fresh prior to the assay using a 10 mM DMSO stock. Final DMSO concentration was 0.5%. The plates were incubated for 3 days at 5% CO2, 37° C. Cell viability was assessed using the CellTiter-Glo assay kit as recommended by the Manufacturer (Promega, Cat. No. G7558, Lot. No. 0000385738) and measurements performed on the EnSpire multimode plate reader (Perkin Elmer). EC50 values were determined using Prism8 (GraphPad) and the asymmetric (five-parameter) equation and least squares fit. GSPT1 levels were assessed in the NCI-H1155 and ABC-1 cell lines by densitometry analysis following treatment with compound 210 for 6 hours and western blotting analysis. Briefly, cells were rinsed with PBS and lysed using 50 μL RIPA lysis buffer (Pierce 8990) supplemented with protease and phosphate inhibitors (Sigma P8340, Sigma 5726, and Sigma 0044). Primary antibody (human GSPT1, Sigma HPA052488) was diluted in blocking buffer (dil. 1/5000) and incubated with the membranes overnight at 4° C. After two rinses with 1×TBST and two washes with 1×TBST (10 minutes each with shaking at room temperature), HRP-coupled secondary antibody (Invitrogen, A16110, dil. 1/5000) and DyLight680-coupled GAPDH antibody (Invitrogen, MA5-15738-D680, dil. 1/2500) were diluted in blocking buffer, added to membranes and incubated with shaking for at least 45 minutes at room temperature protected from light. Membranes were then transferred between two clean sheets of plastic and chemiluminescence (GSPT1), Dylight680 fluorescence (GAPDH) as well as colorimetric (molecular weight standard) signals were detected using a Chemidoc MP imaging system (BioRad 17001402). For chemiluminescence and colorimetric signals “optimal automatic exposure” settings (3×3 binning) were employed while fluorescence was detected using 1-4 seconds exposure time to achieve best GAPDH signals and avoid overexposure. For image analysis “Image Lab” software (BioRad version 6.0.1) was used. If automatic lane and band detection failed, lanes and bands were indicated manually. Default background correction settings were used. Relative GSPT1 protein levels were calculated by setting GSPT1 levels in vehicle (DMSO)-treated control samples to 100% and normalized to the GAPDH singlas. Dose response data was visualized and a four-parameter non-linear regression curve fitting algorithm (Graphpad Prism version 8.3.0) was used to calculate DC₅₀ (value corresponding to 50% reduction in GSPT1 total levels).

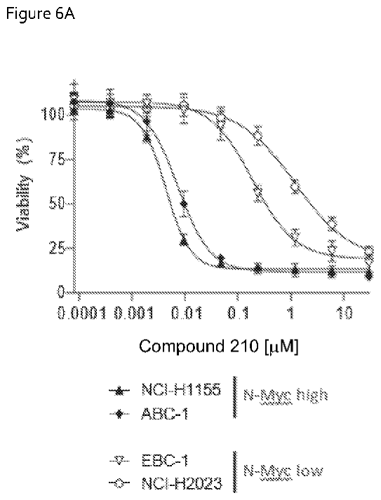

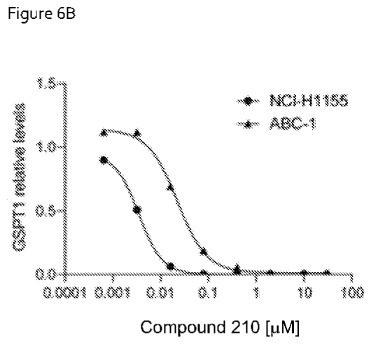

Complete degradation of GSPT1 by compound 210 was observed after six hours of treatment in high N-Myc NCI-Hi 155 and ABC-1 cells with a DC50 of 3 nM and 22 nM, respectively. Compound 210 sensitivity correlated with the expression of N-Myc in the NSCLC cell line (FIG. 6A). Degradation took place in a concentration dependent manner (FIG. 6B).

### Example 10: In Vivoefficacy Study—Tumor Growth Inhibition

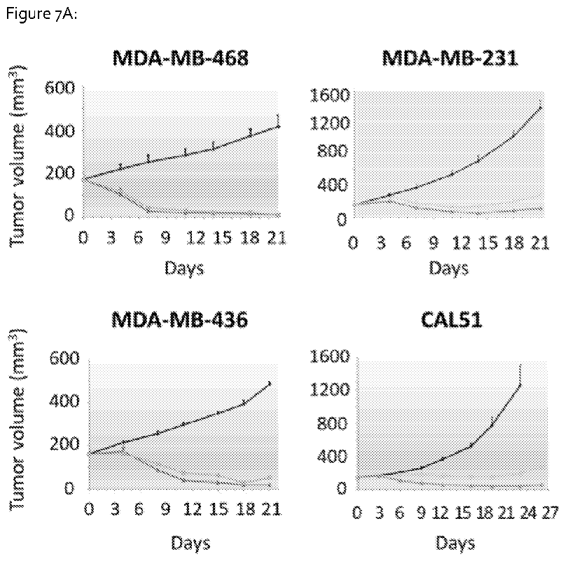

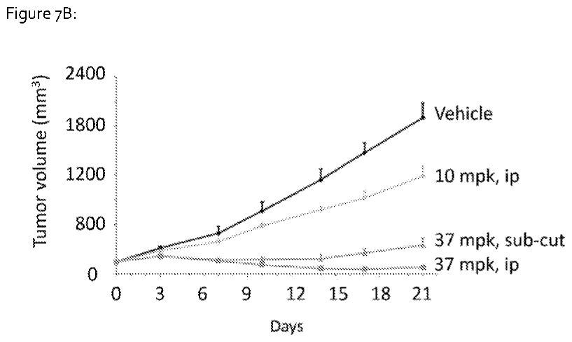

CAL51 (DSMZ-ACC-302), MDA-MB-468 (ATCC HTB-132), MDA-MB-231 (ATCC HTB-26) and MDA-MB-436 (ATCC HTB-130) cells were maintained in vitro in individual medium and conditions as recommended by the providers and incubated at 37° C. in an atmosphere of 5% CO₂ in air. All cell lines were authenticated and confirmed by STR. 10 million cells (5 million for CAL51), resuspended in 0.2 mL of PBS with Matrigel (50:50), were inoculated into Balb/c nude female mice (SCID beige female mice for CAL51) and allowed to grow to 150 mm³ in size. Mice were dosed daily i.p. with either vehicle or compound 8 (at 37 or 75 mg per kilogram). Compound formulations were prepared fresh daily in 0.5% MC4000 and 0.2% Tween80. Mice were dosed for 21 continuous days (26 days for CAL51) and tumor volumes measured every 3 days using the formula: V=0.5 a×b2 where a and b are the long and short diameters of the tumor, respectively. TGI was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment (see FIG. 7).

### Example 11: In Vivo Efficacy Study: Tumor Growth Inhibition in MDA-MB-213 Model (FIG. 7B)

MDA-MB-231 cells were maintained in vitro in DMEM medium supplemented with 20% heat inactivated FBS at 37° C. in an atmosphere of 5% CO₂ in air. Cells (10 millions) resuspended in 0.2 mL of PBS with Matrigel (50:50) were inoculated into female BALB/c nude mice and allowed to grow to 150 mm³ in size. Mice were dosed daily i.p. with vehicle or compound 8 at 10 and 37 mg per kilogram i.p. or 37 mg per kilogram sub-cut. Compound formulations were prepared fresh daily in 5% DMSO/95% (30% HP-β-CD in purified water). Mice were dosed for 21 days or 24 days and tumor volumes measured every 3 days. Results are shown in and FIG. 7B for compound 8 (x-axis: days; y-axis: tumor volume in mm³)

### Example 12 In Vivo Efficacy Studies in N-Myc High NSCLC and SCLC Cell Lines

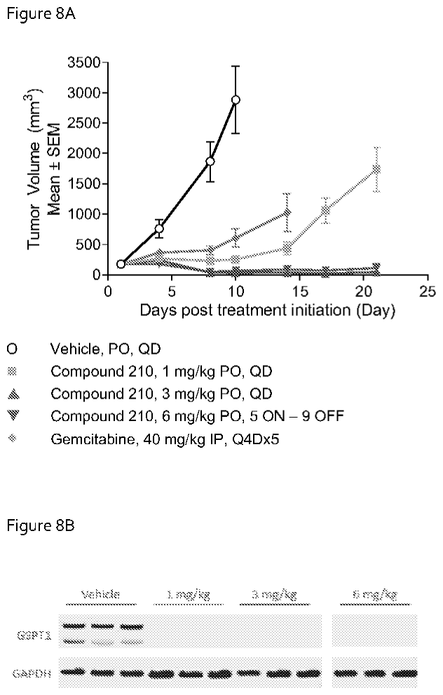

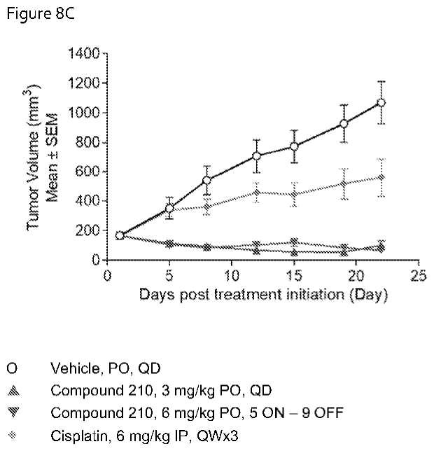

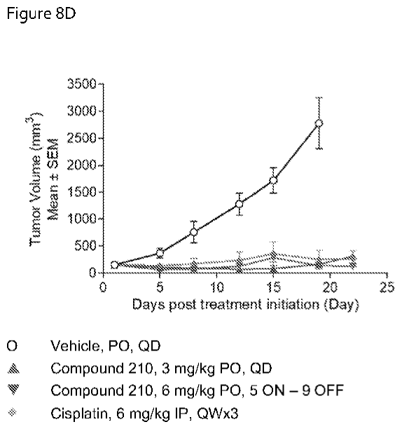

For the NCI-H1155 xenograft study, cells (human non small cell lung cancer, ATCC CRL-5818) were maintained in vitro as suspension culture in ATCC-formulated DMEM:F12 Medium supplemented as recommended by the provider and kept at 37° C. in an atmosphere of 5% CO₂ in air. Cells (0.5 million) were resuspended in 0.2 mL of PBS with Matrigel (50:50) and inoculated into female BALB/c nude mice, 6-8 weeks old, and allowed to grow to 150 mm³ in size. Mice were dosed orally with either vehicle or compound 210 at 1 or 3 mg per kilogram daily (filled squares and filled triangles, respectively) or 6 mg per kilogram for 5 continuous days followed by 9 days treatment holidays (5 On-9 off) (filled inverted triangles). Compound formulations were prepared fresh daily in 5% vDMSO/95% (30% w/v HP-β-CD in water). Gemcitabine dosed at 40 mg per kilogram, IP, Q4D×5 was used as a positive control (filled rhombi). For this study, mice were dosed for 21 days with tumor volumes measured every 3 days. Results are shown in FIG. 8A for compound 210 (x-axis: days; y-axis: tumor volume in mm³) and indicate that oral administration of compound 210 in a N-Myc-driven mouse xenograft model using the human cell line NCI-H1155 led to tumor growth inhibition (with no body weight loss observed). At a dose of 1 mg/kg once daily, tumor growth was suppressed for two weeks. At a dose of 3 mg/kg once daily or 6 mg/kg dosed for five days on and nine days off, tumor size decreased, became undetectable by day eight and remained so until the end of the study at day 21.

GSPT1 levels in tumors were determined by western blotting 6 hours after the third dose. Western blotting was performed as described previously using GAPDH as loading control. Complete degradation of GSPT1 was observed in tumors of mice treated with compound 210 at all three dose levels as compared to mice treated with vehicle control (FIG. 8B).

For the NCI-H1770 study, cells (human non small cell lung cancer, ATCC CRL-5893, Lot No. 5188933) were maintained in vitro as suspension culture in ATCC-formulated RPMI-1640 Medium supplemented by 10% FBS and cultured at 37° C. in an atmosphere of 5% CO₂ in air. Cells (10.0 million) were resuspended in 0.2 mL of PBS with Matrigel (50:50) and inoculated into female BALB/c nude mice, 6-8 weeks old, and allowed to grow to 150 mm³ in size. Mice were dosed orally with either vehicle (empty circles) or compound 210 at 3 mg per kilogram daily or 6 mg per kilogram for 5 continous days followed by 9 days treatment holidays (5 On-9 off) (filled triangles and filled inverted triangles, respectively). Compound formulations were prepared fresh daily in 5% vDMSO/95%(30% w/v HP-β-CD in water). Cisplatin dosed at 6 mg per kilogram, IP, QW×3 was used as a positive for this model (filled rhombi). Mice were dosed for 21 days with tumor volumes measured every 3 days. Similar results were observed as for NCI-H1155 as shown in FIG. 8C for compound 210 (x-axis: days; y-axis: tumor volume in mm³).

For the NCI-H526 study, cells (human small cell lung cancer, ATCC CRL-5811) were maintained in vitro as suspension culture in ATCC-formulated RPMI-1640 Medium supplemented with 10% FBS and cultured at 37° C. in an atmosphere of 5% CO₂ in air. Cells (2.5 million) were resuspended in 0.2 mL of PBS with Matrigel (50:50) and inoculated into female BALB/c nude mice, 6-8 weeks old, and allowed to grow to 150 mm³ in size. Mice were dosed orally with either vehicle (empty circles) or compound 210 at 3 mg per kilogram daily or 6 mg per kilogram for 5 continous days followed by 9 days treatment holidays (5 On-9 off) (filled triangles and filled inverted triangles, respectively). Compound formulations were prepared fresh daily in 5% vDMSO/95% (30% w/v HP-β-CD in water). Cisplatin dosed at 6 mg per kilogram, IP, QW×3 was used as a positive for this model (filled rhombi). Mice were dosed for 21 days with tumor volumes measured every 3 days. Similar results were observed as for NCI-H1155 as shown in FIG. 8D for compound 210 (x-axis: days; y-axis: tumor volume in mm³).

### Example 13: CK1Alpha/Ikaros/Aiolos/ZFP91 Selectivity Determination by Western Blot Assay

MM1 S cells were purchased from ATCC (cat. Number CRL-2974), sub-cultured in 90% RPMI 1640 with 10% FBS, supplemented with 1×P/S and incubated at 37° C., 5% CO2. Compounds were stored as 10 mM DMSO stock. For the assay, MM1 S cells (3 million cells/well) were plated in 6-well plates and incubated over night. Cells were treated with respective compounds using a serial dilution: 0.3 μM, 3 μM and 30 μM as well as a vehicle only (DMSO) control for 6 hours. Media with suspension cells was subsequently transferred to 15 mL conical tubes, wells rinsed twice with ice-cold PBS and merged with cell suspension in respective 15 mL conical tube. Cells were spinned down, supernatant aspirated, pellets resuspended in ice-cold PBS and transferred to microtubes. Cells were spinned down, supernatant aspirated and pellets resuspended in 120 μL RIPA lysis buffer supplemented with protease and phosphatase inhibitors. Cell lysates were incubated on ice for 20 minutes followed by centrifugation at >20,000×g for 5 min. Supernatants were transferred to fresh microtubes and stored at −80° C. Total protein concentration was determined using a BCA assay with a BSA standard curve and concentration of all samples was adjusted to 1 mg/mL. 25 μL 4×LDS sample buffer supplemented with 100 mM DTT was added to 75 μL sample. Samples were centrifuged (8,000×g, 1 min) and incubated at 95° C. for 5 min followed by another centrifugation step (8,000×g, 1 min). 20 μL of each sample was loaded on a 4-12% gel alongside a protein molecular weight marker. Gels were run in the presence of MOPS buffer at 80 Volts for 30 min, followed by 120 Volts for 1.5 h and proteins subsequently transferred onto nitrocellulose membranes at 20 Volts for 7 min using an iBlot2 Gel Transfer Device. Membranes were then cut horizontally into two pieces, covering 80-50 kDa and 50-25 kDa. Blocking the membranes was performed by gently shaking in 5% (w/v) skim milk in TSB-T for 1 hr at room temperature. All primary antibodies were used at a 1/1000 in 5% (w/v) BSA dissolved in TBST and incubated with membranes over night at 4° C. After three washes with 1×TBST for 5 min, HRP-coupled secondary antibodies diluted in 5% (w/v) BSA/TBST (Goat Anti-Rb IgG, dil. 1/10,000; Goat Anti-Mouse IgG, dil. 1/5000) were added for 1 hr at room temperature. After three washes with 1×TBST (5 minutes each), membranes were incubated with ECL reagent for 1 min at room temperature. Chemiluminescence signals were then detected using a LAS-4000 system with default settings and signals quantified using Image Studio Lite software (version 5.2). Membrane parts previously incubated with antibody against CK1 alpha were stripped off antibodies by incubating with stripping buffer for 30 minutes followed by three washed with TBST (5 min each), blocking with 5% (w/v) skim milk for 1 hr, and incubation with primary antibody against uGAPDH overnight at 4° C. Subsequent washes, incubation with secondary antibody and signal acquisition were performed as described above.

Table 7 assigns each compound a code indicating the ability for the degradation of IKZF1, IKZF3, CK1 alpha and ZFP91: A, B, C, D, E or F. According to the code, A represents no degradation observed at 30 μM, B represents trace degradation at 30 μM (below 20%), C represents weak degradation at 30 μM (below 50%), D represents degradation at 30 μM (>90%), E represents degradation at 3 μM (>90%) and F represents degradation at 0.3 μM (>90%).

TABLE 7
Selectivity for relevant Zincfinger proteins:

| Compound | IKZF1 | IKZF3 | CK1alpha | ZFP91 | GSPT1 |
|---|---|---|---|---|---|
| 8 | A | B | A | B | F |
| 75 | A | A | A | A | E |
| 82 | A | A | A | A | F |
| 85 | A | A | B | A | E |
| 87 | A | A | A | A | E |
| 105 | B | A | A | B | E |
| 132 | A | A | C | C | F |
| 138 | C | B | A | A | F |
| 201 | A | B | B | A | F |
| 203 | A | A | B | B | F |
| 206 | A | B | A | | F |
| 210 | A | B | B | | D |
| 215 | A | B | C | | F |
| 242 | A | A | B | | F |
| 243 | A | B | C | | F |
| 247 | A | A | B | B | F |
| 248 | A | B | B | | F |
| 249 | A | A | B | B | F |

### Example 14: GSPT1 Levels in Tumors

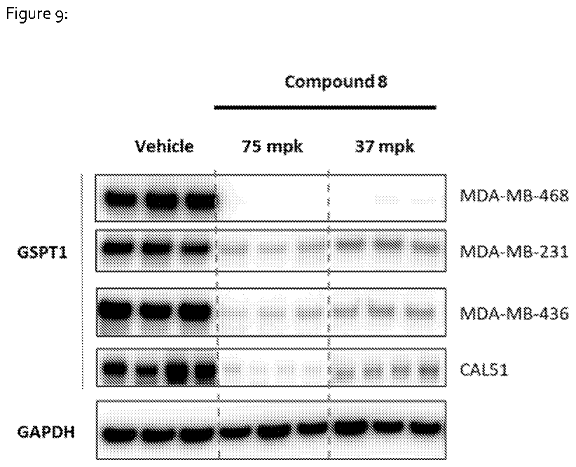

For the PD analysis, tumors were harvested 24 hr post third dose and GSPT1 total levels in tumors were determined by western blotting analysis. GADPH levels were measured as control Briefly, 30-50 mg of tumor was placed in 350 μL of RIPA buffer containing 1% Protease Inhibitor Cocktail and 1% Phosphatase Inhibitor Cocktail 2. Tumors were grinded using the Tissuelyser LT at 50 Hz for 5 min. the tissue lysates were kept on ice for 30 min, centrifuged at 12,000 rpm at 4° C. for 10 min and protein concentration measured using the Pierce™ BCA Protein Assay Kit as recommended by the manufacturer. Samples were diluted to the same final concentration (at 4 μg/μL) using RIPA buffer plus 4×LDS Sample Buffer and 10× Sample Reducing Agent and subsequently heat denatured at 100° C. for 10 min. Western blot analysis was performed using 5 μL per slot for each sample, loaded on a NuPAGE® 4-12% Bis-Tris gel, probing for GSPT1 (Sigma-HPA052488; 1/250 dilution) and GAPDH as loading control (CST-5174; 1/2000 dilution). FIG. 9 shows that no or insignificant amounts of GSPT1 for mice that received 75 mg/kg and 37 mp/kg of compound 8, respectively, and high GSPT1 levels for mice that did not receive any active compound 8.

### Example 15: Induced-Degradation of GSPT1 Following Treatment with Compound 345 in NSCLC Cancer Cell Lines

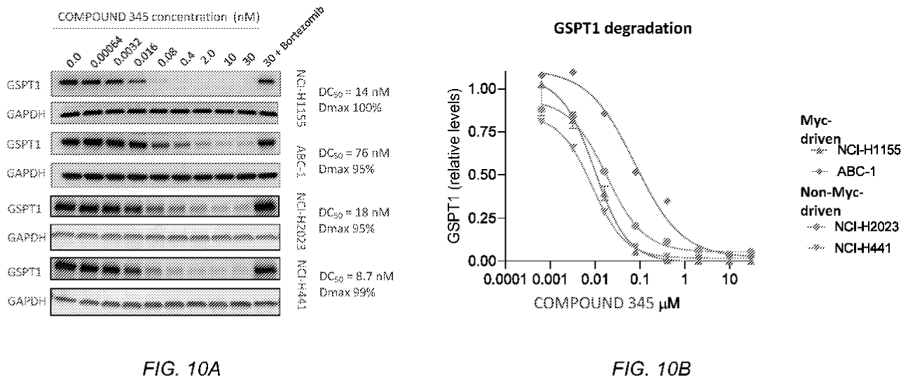

Two high N-Myc expressing NSCLC cell lines (NCI-H155 and ABC-1) and two low N-Myc expressing NSCLC cell lines (NCI-H2023 and NCI-H441) were treated with Compound 345 in culture. Cells were treated with increasing concentrations of the compound (0.0; 0.00064; 0.0032; 0.016; 0.08; 0.4; 2.0; 10; and 30 nM), lysates were prepared, SDS-PAGE and immunoblotting was performed, and transfer membranes were probed for GSPT1 and GAPDH as a loading control. Representative immunoblots from this experiment are shown in FIG. 10A. Expression levels of GSPT1 detected in the immunoblots of this experiment are plotted against concentration of Compound 345 in FIG. 10B. DC₅₀ values (the concentration of the compound at which the target protein is degraded by 50%) were estimated for each cell line. NCI-H155, DC₅₀=14 nM; ABC-1, DC₅₀=76 nM; NCI-H2023, DC₅₀=18 nM; NCI-H441, DC₅₀=8.7 nM. Estimated Dₘₐₓ for all cell lines was 95%-99%. This experiment demonstrated that Compound 345 effectively induces degradation of GSPT1 in high N-Myc expression and low N-Myc expressing NSCLC cell lines.

### Example 16: Induced-Degradation of GSPT1 Following Treatment with Compound 345 and Anti-Proliferative Activity in NSCLC Cancer Cell Lines

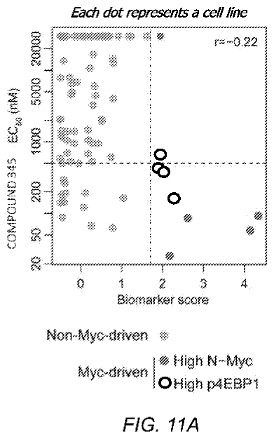

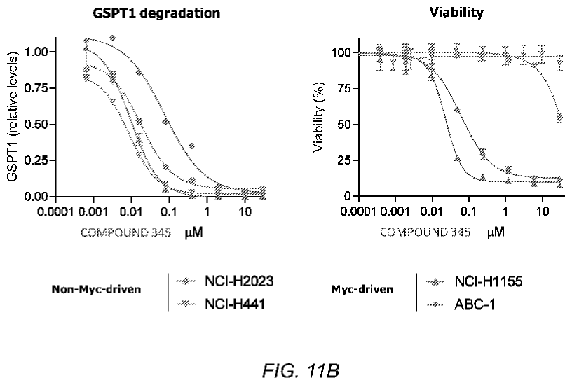

EC₅₀ scores for Compound 345 were measured in a panel of NSCLC cell lines. Each cell line was scored for NSCLC biomarkers and EC₅₀ values were plotted against NSCLC biomarker score. Results of this experiment are shown in the scatter plot of FIG. 11A. High N-Myc expressing cell lines were enriched in the lower right quadrant of the scatterplot, indicating high NSCLC biomarker scores and high sensitivity to Compound 345. Additionally, Compound 345 was shown to degrade GSPT1 in Myc-driven and non-Myc-driven NSCLC, however, cell viability was significantly reduced in only Myc-driven NSCLC as shown in FIG. 11B. These results indicate that Myc-driven NSCLC cell lines are highly sensitive to Compound 345.

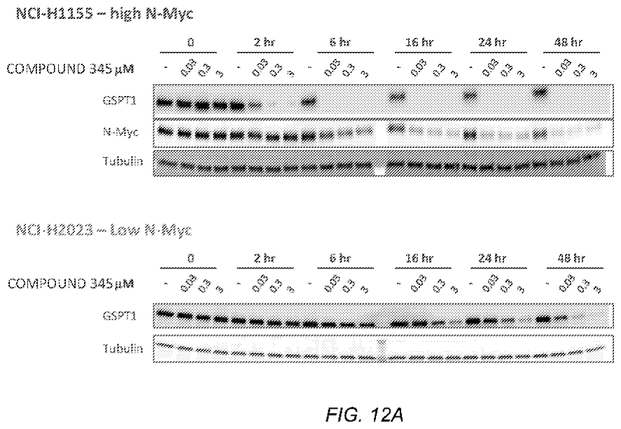

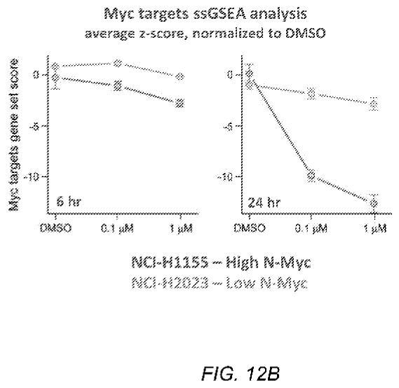

Example 17: Time Course Experiment Following the Degradation of GSPT1 and Concomitant Downregulation in NSCLC Cancer Lines Following Treatment with Compound 345. A High N-Myc expressing NSCLC cell lines (NCI-H155) and a low N-Myc expressing NSCLC cell lines (NCI-H2023) were treated with Compound 345 in culture. Cells were treated with increasing concentrations of the compound (0.0; 0.03; 0.3; 3 μM), cells were withdrawn from the culture at time points of 0, 2, and 6 hours, lysates were prepared, SDS-PAGE and immunoblotting was performed, and transfer membranes were probed for GSPT1, N-Myc, and Tubulin as a loading control (N-Myc was not probed in the NCI-2023 experiment). Representative immunoblots from this experiment are shown in FIG. 12A. The immunoblots show efficient degradation of GSPT1 in a time and dose dependent manner in response to treatment with Compound 345. For the same cell lines, RNAseq followed by single sample gene set enrichment analysis (ssGSEA) was performed at six hours and 24 hours after treatment with Compound 345. Results of this experiment are shown in FIG. 12B. ssGSEA scores for Myc target genes are plotted against Compound 345 concentration normalized to DMSO at six hours after treatment (left plot) and 24 hours after treatment (right plot). At six hours there is a modest down regulation of the Myc gene set in the high N-Myc expressing cells at 0.1 μM and 1 μM doses. After 24 hours there is a significant downregulation of the Myc gene set in the high N-Myc expressing cells at 0.1 μM and 1 μM doses. This experiment demonstrates that Compound 345 efficiently degrades GSPT1 and downregulates the Myc pathway in high N-Myc expressing NSCLC cell lines.

### Example 18: Compound 345 Induces Tumor Regression in N-Myc-Driven Xenograft Models of NSCLC

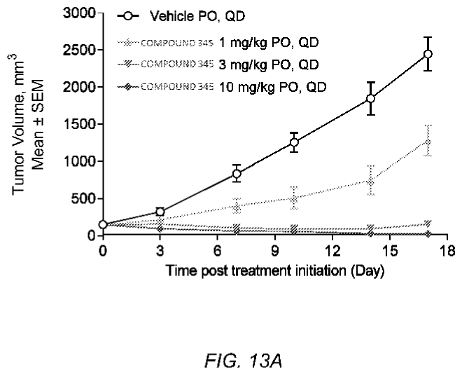

Cell line derived xenograft (CDX) models were produced for the NCI-1155 NSCLC cell line in immunodeficient mice. Oral dosing of Compound 345 was performed daily at doses of 1 mg/kg, 3 mg/kg, and 10 mg/kg with a no-drug vehicle control. Tumor volumes were measured (mm³) at days 3, 7, 10, 14, and 17. Results from this experiment are shown in FIG. 13A. This experiment demonstrates that oral dosing of Compound 345 shows anti-tumor activity and regression in a NCI-1155 CDX model.

Additionally, plasma concentrations of Compound 345 and levels of GSPT1 and M-Myc (relative to vehicle-only control) were measured in a time-course experiment on day 5 for the animals receiving 1 mg/kg and 10 mg/kg. Results of this time course experiment are shown in FIG. 13B. GSPT1 and N-Myc are levels are substantially reduced relative to vehicle-only control, with the strongest effect at the 10 mg/kg dose. This experiment demonstrates that dose and time dependent degradation of GSPT1 by treatment with Compound 345 is associated with N-Myc downregulation.

### Example 19: Anti-Tumor Activity of Compound 345 in Patient Derived Xenograft Models of NSCLC

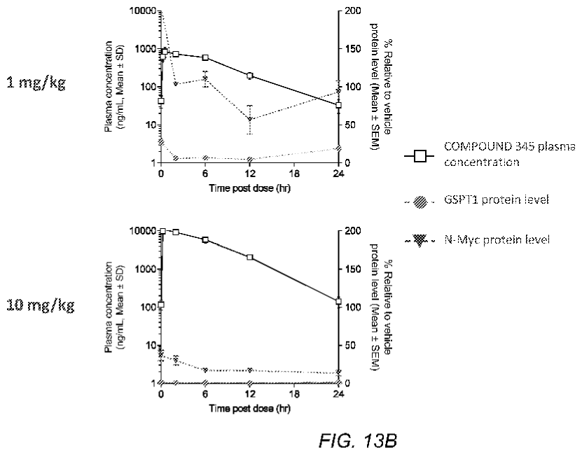

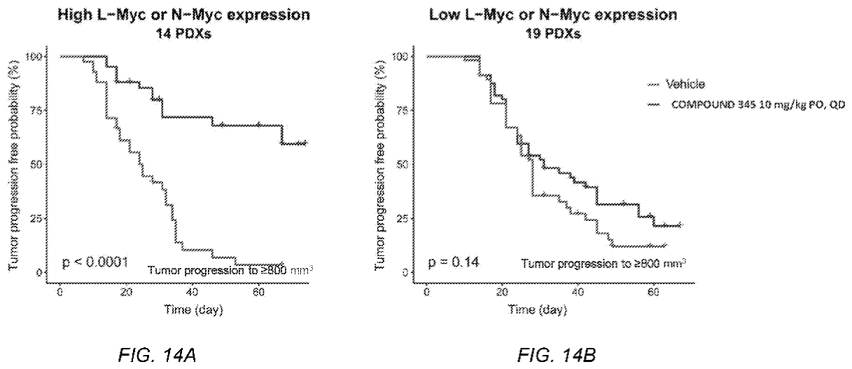

Patient derived NSCLC xenograft models (PDX) were made using immunodeficient mice. 14 PDXs were made for high L-Myc or N-Myc expressing NSCLC samples and 19 PDXs were made for low L-Myc or N-Myc expressing NSCLC samples. Animals were divided into a treatment group that received an oral dose of 10 mg/kg Compound 345 daily and a vehicle-only control group. Animals were observed for up to 80 days and tumor progression was measured at regular intervals. Data from these experiments are shown in FIG. 14A (high L-Myc or high N-Myc expressing) and FIG. 14B (low L-Myc or low N-Myc expressing). Tumor progression free probability was plotted over time, with a threshold of tumor progression set at greater than 800 mm³. In the high L-Myc or N-Myc expressing models, treatment with Compound 345 significantly extended the probability that an animal remained tumor-free.

### Example 20: Compound 345 Induces Degradation of GSPT1 and Associated with L-Myc Downregulation in Myc High SCLC Cells Lines

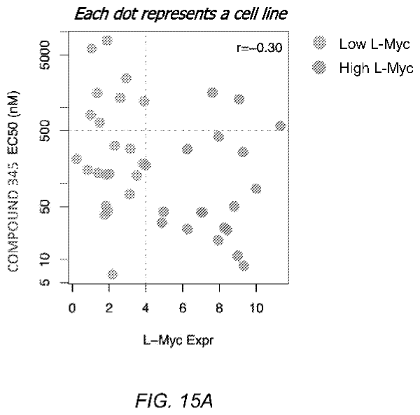

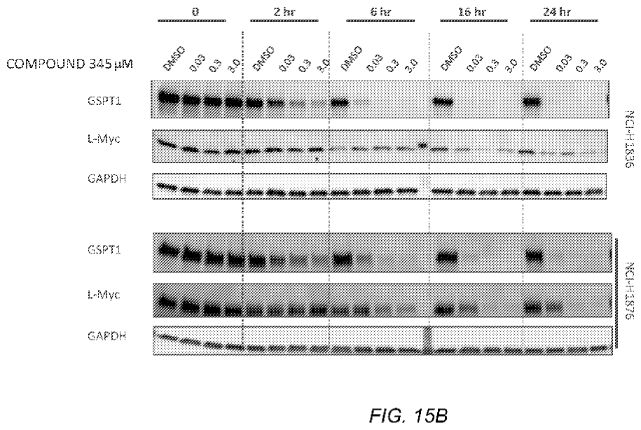

FIG. 15A shows the anti-proliferative activity of Compound 345 against a panel of SCLC cancer cell lines and association with L-Myc (red) levels. Two high L-Myc expressing small cell lung cancer (SCLC) cell lines (NCI-H1836 and NCI-H1876) were treated with Compound 345 in culture. Cells were treated with increasing concentrations of the compound (0.0; 0.03; 0.3; 3 μM), cells were withdrawn from the culture at time points of 0, 2, 6, 16, and 24 hours, lysates were prepared, SDS-PAGE and immunoblotting was performed, and transfer membranes were probed for GSPT1, L-Myc, and GAPDH as a loading control. Representative immunoblots from this experiment are shown in FIG. 15B. The immunoblots show efficient degradation of GSPT1 in SCLC cell lines in a time and dose dependent manner in response to treatment with Compound 345.

### Example 21: Degradation of GSPT1 is Associated with c-Myc Downregulation and Apoptosis in MM

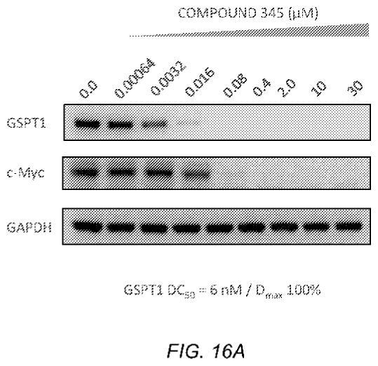

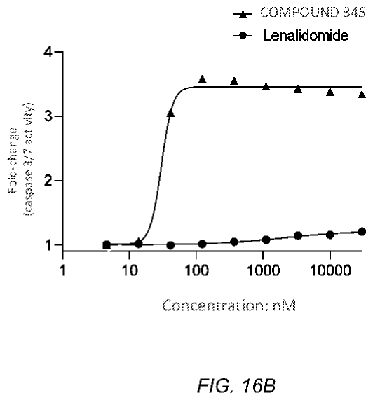

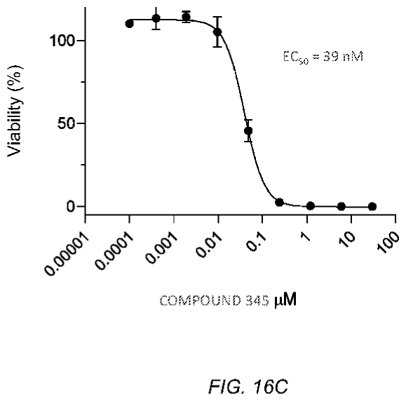

A c-Myc expressing multiple myeloma (MM) cell line (MM1 S) was treated with Compound 345 in culture. Cells were treated with increasing concentrations of the compound (0.0; 0.00064; 0.0032; 0.016; 0.08; 0.4; 2.0; 10; and 30 μM) and after 24 hours lysates were prepared, SDS-PAGE and immunoblotting was performed, and transfer membranes were probed for GSPT1, c-Myc, and GAPDH as a loading control. Representative immunoblots from this experiment are shown in FIG. 16A. GSPT1 DC₅₀ was estimated at 6 nM and Dₘₐₓ was 100%. The immunoblots show efficient degradation of GSPT1 in the MM cell line in a dose dependent manner in response to treatment with Compound 345.

For the same MM cell line, the effect of Compound 345 on apoptosis was evaluated. MM1 S cells were treated with Compound 345 in culture at increasing concentrations ranging from 0 to 30 μM and a control group was treated with lenalidomide, a well-known MM drug. After 48 hours, fold-change caspase 3/7 activity (a marker of apoptosis) was measured and plotted against concentration of Compound 345 compared to lenalidomide as the control. Results from this experiment are shown in FIG. 16B. The results indicate that Compound 345 promotes a strong induction of apoptosis in MM cells. For the same MM cell line, the impact of Compound 345 on cell viability was evaluated. MM1 S cells were treated with Compound 345 in culture at increasing concentrations ranging from 0 to 100 μM. Cell viability was measured as a percentage survival after 72 hours. Results from this experiment are shown in FIG. 16C.

### Example 22: Compound 345 Induces Tumor Regression in c-Myc-Driven Xenograft Model of MM

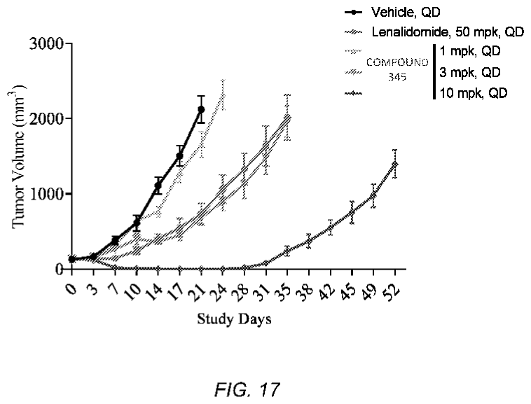

Cell line derived xenograft (CDX) models were produced for the MM1 S multiple myeloma (MM) cell line in immunodeficient mice. Oral dosing of Compound 345 was performed daily at doses of 1 mg/kg, 3 mg/kg, and 10 mg/kg as well as lenalidomide at 50 mg/kg and a no-drug vehicle control. Tumor volumes were measured (mm³) at regular intervals over the course of 20 to 50 days depending on the animal. Results from this experiment are shown in FIG. 17. This experiment demonstrates that oral dosing of Compound 345 shows anti-tumor activity in a c-Myc-driven MM CDX model.

### Example 23: Compound 345 Activity in a Triple Hit Lymphoma Model

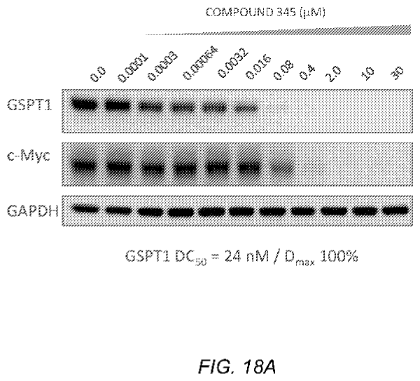

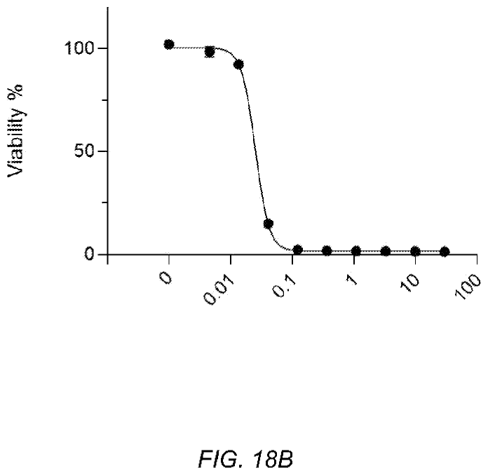

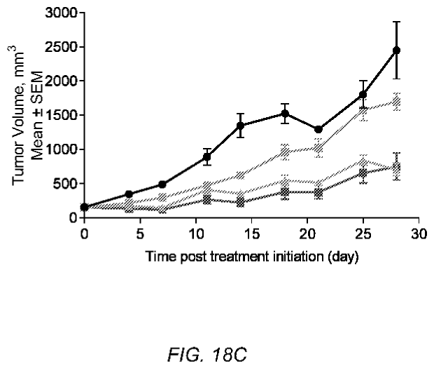

A c-Myc expressing triple hit lymphoma cell line (WSU-DLCL2) was treated with Compound 345 in culture. Cells were treated with increasing concentrations of the compound (0.0; 0.0001; 0.0003; 0.00064; 0.0032; 0.016; 0.08; 0.4; 2.0; 10; and 30 μM) and after 24 hours lysates were prepared, SDS-PAGE and immunoblotting was performed, and transfer membranes were probed for GSPT1, c-Myc, and GAPDH as a loading control. Representative immunoblots from this experiment are shown in FIG. 18A. GSPT1 DC₅₀ was estimated at 24 nM and Dₘₐₓ was 100%. The immunoblots show efficient degradation of GSPT1 in the triple hit lymphoma cell line in a dose dependent manner in response to treatment with Compound 345. FIG. 18B shows the antiproliferative activity of Compound 345 (as indicated) following treatment with Compound 345 for 72 hr.

Additionally, cell line derived xenograft (CDX) models were produced for the triple hit lymphoma cell line (WSU-DLCL2) in immunodeficient mice. Oral dosing of Compound 345 was performed daily at doses of 3 mg/kg and 10 mg/kg as well as the CHOP combination (a well-known lymphoma treatment regimen) and a no-drug vehicle control. Tumor volumes were measured (mm³) at regular intervals over the course of up to 30 days depending on the animal. Results from this experiment are shown in FIG. 18C. This experiment demonstrates that oral dosing of Compound 345 shows anti-tumor activity in a c-Myc-driven triple hit lymphoma CDX model.

### Example 24: Activity of Compound 345 in the cMyc High DOHH-2 Lymphoma Cell Line

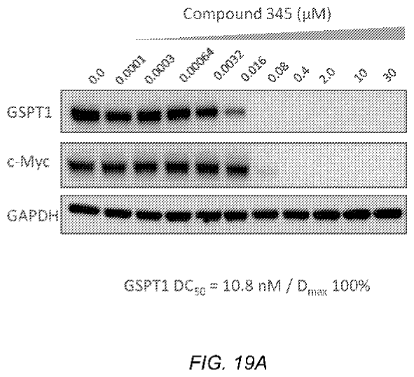

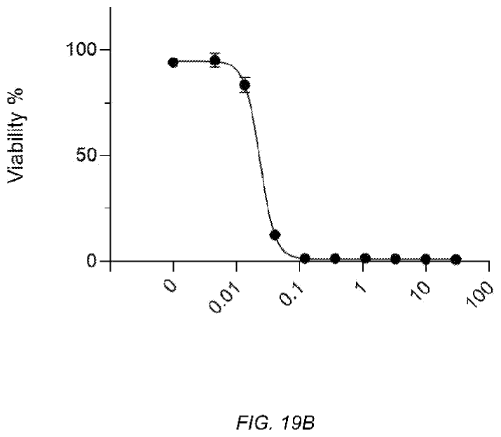

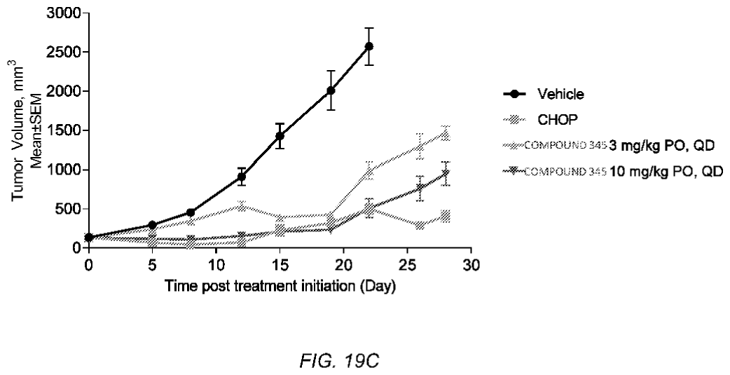

A c-Myc expressing lymphoma cell line (DOHH2) was treated with Compound 345 in culture. Cells were treated with increasing concentrations of the compound (0.0; 0.0001; 0.0003; 0.00064; 0.0032; 0.016; 0.08; 0.4; 2.0; 10; and 30 μM) and after 24 hours lysates were prepared, SDS-PAGE and immunoblotting was performed, and transfer membranes were probed for GSPT1, c-Myc, and GAPDH as a loading control. Representative immunoblots from this experiment are shown in FIG. 19A. Antiproliferative activity of Compound 345 (as indicated) following treatment with Compound 345 for 72 is shown in FIG. 19B.

Additionally, cell line derived xenograft (CDX) models were produced for lymphoma cell line (DOHH2) in immunodeficient mice. Oral dosing of Compound 345 was performed daily at doses of 3 mg/kg and 10 mg/kg as well as the CHOP combination (a well-known lymphoma treatment regimen) and a no-drug vehicle control. Tumor volumes were measured (mm³) at regular intervals over the course of up to 30 days depending on the animal. Results from this experiment are shown in FIG. 19C. This experiment demonstrates that oral dosing of Compound 345 shows anti-tumor activity in a c-Myc-driven double hit lymphoma CDX model.

### Example 25: Activity of Compound 345 in Multiple Melanoma Cell Lines

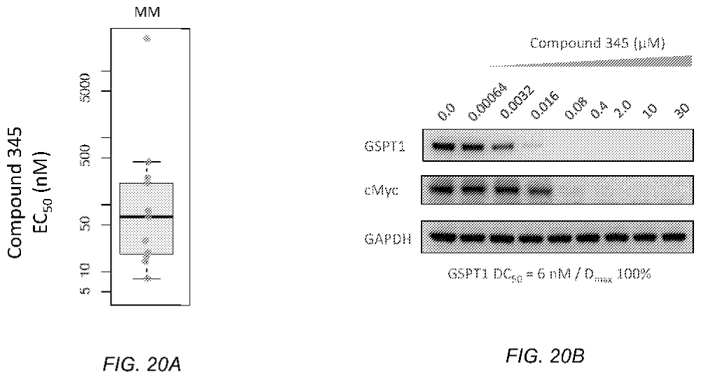

The anti-proliferative activity of Compound 345 against a panel of multiple myeloma:cancer cell lines. The results of this analysis are presented in FIG. 20A. As note previously, Compound 345 causes degradation of GSPT1 and concomitant downregulation of c-Myc following in the MM1 S multiple myeloma cancer cell line (FIG. 20B and FIG. 16A).

### Example 26: Anti-Proliferative Activity and GSPT1 Downregulation by Compound 345 in Lymphoma Cancer Cell Lines

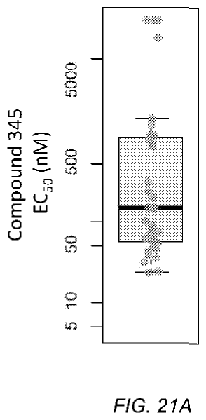

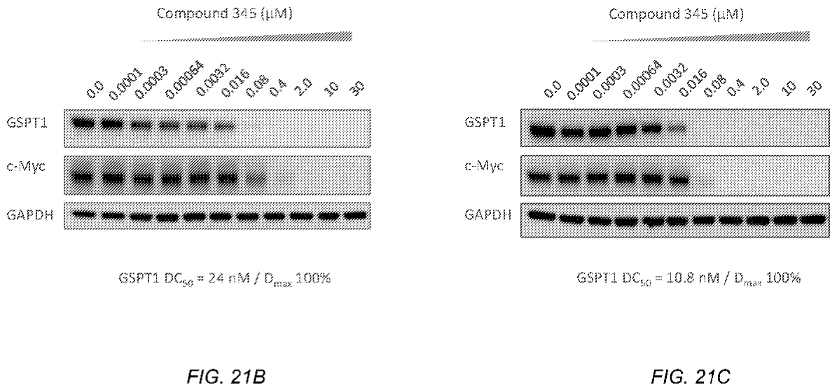

The EC50 of Compound 345 was measured in a panel of lymphoma cancer cell lines. The results of this analysis are shown in FIG. 21A. The degradation of GSPT1 and concomitant downregulation of cMyc following treatment with Compound 345 in the WSU-DLCL2 and DOHH-2 lymphoma cancer cell lines is shown in FIG. 211B and FIG. 21C, respectively.

### Experimental Protocols for Examples 15-26

The studies in Examples 15-26 were carried out essentially as described below.

Viability assay: Compound 345 was tested in a panel of 156 lung cancer cell lines (including non small cell lung cancer (NSCLC) and small cell lung cancer (SCLC) cell lines and 46 lymphoma cell lines using a 72 hr CellTiter Glo assay format. Cells were sub-cultured as recommended by the providers (ATCC, DSMZ) and incubated at 37° C., 5% CO2. All cells were authenticated and confirmed by Short Tandem Repeat (STR) profiling. Briefly, each lung cancer cell line was preferentially seeded in 384-well plates (100 μL/well) at a density previously determined to be in log growth phase 72 hour post-seeding (typically in the 400-2400 cells/well range). Lymphoma cell lines growing in suspension were preferentially seeded in 96-well plates (100 μL/well) typically in the 6000-20000 cells/well range. Cells were incubated overnight in a humidified incubator at 37° C. with 5% CO2. Compound 345 and DMSO control were subsequently dispensed to the plates using a HP D300 dispenser (Tecan) and a 9 data-point titration curve with concentrations ranging from 30 μM to 0.1 nM. Dilutions were prepared fresh prior to the assay using a 10 mM DMSO stock. Final DMSO concentration was 0.5%. The plates were incubated for 3 days at 5% CO2, 37° C. Cell viability was assessed using the CellTiter-Glo® 2.0 Assay as recommended by the Manufacturer (Promega, Cat. No. G7558, Lot. No. 0000385738) and measurements performed on the EnSpire multimode plate reader (Perkin Elmer). EC50 values were determined using Prism8.1.2 (GraphPad) and the asymmetric (five-parameter) equation and least squares fit.

For follow-up studies, NCI-H1155 (ATCC, Cat. No. CRL-5818), NCI-H2023 (ATCC, Cat. No. CRL-5912), NCI-H441 (ATCC, Cat. No. HTB-174), ABC-1 (Japanese Cancer Research Resources Bank JCBR, Cat. No. JCRB0815), NCI-H1836 (ATCC, Cat. No. CRL-5898), NCI-H1876 (ATCC, Cat. No. CRL-5902), MM.1S (ATCC, Cat. No. CRL-2974), WSU-DLCL2 (DSMZ, Cat. No. ACC-575) and DOHH2 (DSMZ, Cat. No. ACC-47) were cultured as recommended by the providers and seeded in white 384 or 96 well plate (cell density typically between 500 to 12500 cells/mL for 80-100 μL/well in 384 well plate; and between 6000-20000 cells/mL for 100-150 μL/well in 96 well plate) and incubated overnight. Wells located on the outer part of the plate were filled with media only and will be used a blanks for the experiment. Compound 345 was dissolved in DMSO to obtain a 10 mM stock solution, and aliquots were stored at −20° C. The next day, cells were treated in triplicates with various concentrations of Compound 345 (typically 0.1 nM to 30 μM) or DMSO (Sigma, D848) using a HP D300 dispenser (Tecan) and placed in the incubator for 72 hours. Cell viability was assessed using the CellTiter-Glo® 2.0 Assay (Promega, G9243). Briefly, assay plates and CellTiter-Glo reagents were equilibrated at room temperature for thirty minutes. Addition of 10 to 15 μL CellTiter-Glo reagent per well was followed by 20 min incubation under vigorous agitation in the dark. Luminescence readout was captured on a PHERAstar® FSX multi-mode plate reader (BMG LabTech). At least two independent experiments were performed. EC50 values were determined using Prism8.1.2 (GraphPad) and the asymmetric (five-parameter) equation and least squares fit).

GSPT1 and N-Myc western blotting analyses from cell line lysates: NCI-H1155, NCI-H2023, NCI-H441, ABC-1, NCI-H1836, NCI-H1876, MM.1S, DOHH2 and WSU-DLCLC2 cells were typically seeded in a 6 or 12 well plate (0.3 to 1.0×106 cells/mL) and incubated overnight. Compound 345 was dissolved in DMSO to obtain a 10 mM stock solution, and aliquots were stored at −20° C. The next day, cells were treated with various concentrations of Compound 345 or DMSO (Sigma, D2650) using a HP D300 dispenser (Tecan). Controls were pretreated for 30 minutes with Bortezomib (0.2 μM). Cells were washed with ice cold PBS at the indicated timepoints post addition of Compound 345 and lysed with 50 to 100 μL lysis buffer containing RIPA buffer (Invitrogen, R0278) and a mix of protease inhibitor cocktail (Sigma, P8340, 5726 and 0044). Protein concentration was assessed using the BCA assay (Pierce™ Rapid Gold BCA Protein Assay Kit, A53227) and 10 μg of protein was typically loaded onto a TGX Stain free 4-20% Criterion gel (Biorad, 5678095) for separation. Gel was transferred onto a 0.2 μm nitrocellulose membrane using the TransBlot transfer system (Biorad). Membranes were blocked in Every Blot Blocking Buffer (Biorad, 12010020) and incubated overnight at 4° C. with either of the following primary antibody: anti-GSPT1 diluted 1/5000th (Sigma, HPA052488), anti-N-Myc clone D1V2A diluted 1/1000th (Cell Signaling Technology, 84406), anti-α-Tubulin clone DM1A Alexa Fluor® 488 Conjugated (Cell Signaling Technology, Cat. No. CST8058) or anti-GAPDH Alexa647-coupled diluted 1/2500th (Invitrogen, MA5-15738-A647). The next day, a secondary HRP-conjugated goat anti-rabbit secondary antibody (Invitrogen, A16110) was used to allow detection of unlabelled primary antibodies. Membranes were imaged on a ChemiDoc imager (Biorad) using either fluorescence or chemiluminescence detection. Images were quantified using the Image Lab Software Version 6.1 (Biorad). Images were adjusted for alignment and contrast. Columns were manually defined to enable the band detection by the software. Band intensities, or densitometries, were exported to Excel (Microsoft® Excel® for Microsoft 365 MSO Version 2110) for further analysis. Briefly, band intensity for the protein of interest was normalized to the DMSO control, before being normalized to the GAPDH band intensity for the same column. Normalised data was transferred to GraphPad Prism 8.1.2 (GraphPad) and fitted using a four parameters models ([Inhibitor] vs. response—Variable slope) defined by the following equation: Y=Dmax+(Top−Dmax)/(1+(DC50/X)\hat{ }HillSlope). Key degradation parameters reported included DC50 (concentration at which the GSPT1 total protein levels are reduced by 50%) and Dmax (maximum fractional amount of protein degraded).

Apoptosis assay: MM1S cells were sub-cultures as recommended by the providers (ATCC, DSMZ) and incubated at 37° C., 5% CO2. Briefly, cells were seeded in white 96-well plates (100 μL/well) at a density of 50000 cells/well and incubated overnight at 37° C. with 5% CO2. The compound and the DMSO control were subsequently dispensed to the plates in a 9 data-point titration with concentrations ranging from 30 μM to 4.5 nM. Dilutions were prepared fresh prior to the assay using a 10 mM DMSO stock. Final DMSO concentration was 0.5%. The plates were incubated for 3 days at 5% CO2, 37° C. Cell apoptosis was assessed using the Caspase-Glo® 3/7 Assay kit as recommended by the manufacturer (Promega, Cat. No. G8091, Lot. No. 0000385738) and measurements were performed on the EnSpire multimode plate reader (Perkin Elmer). EC50 values were determined using Prism8.1.2 (GraphPad) and the asymmetric (five-parameter) equation and least squares fit.

Transcriptomics experiment in NCI-H1155 and NCI-H2023 NSCLC lines: NCI-H1155 and NCI-H2023 were typically seeded in a 6 well plate (0.5×106 NCI-H1155 cells/mL; 0.3×106 NCI-H2023 cells/mL; 2 ml/well) and incubated overnight. The next day, cells were treated with various concentrations of Compound 345 or 0.1% DMSO (Sigma, D2650) and incubated for 6 h or 24 h. Compound dilutions were prepared fresh prior to the assay using a 10 mM DMSO stock. Cells were washed with ice cold DPBS (Pan-Biotech, P04-36500), and collected into Nunc™ 96-Well Polypropylene DeepWell™ Storage Plates (Thermo Fisher, 278743). Cell pellets were then snap-frozen in dry ice and stored at −80° C. Cell pellets were thawed, and total RNA was isolated using a MagMax mirVana Total RNA Isolation Kit (Thermo Fisher, A27828) and following manual extraction user guide. Total RNA yields and quality were measured using the DeNovix DS-11 Spectrophotometer. Sequencing libraries were prepared using 500 ng/sample total RNA while following the “Illumina Stranded mRNA Prep Ligation” reference guide and corresponding kits (Illumina, 20040534, Illumina 20040553). Library yields were measured using a QuBit 4 fluorometer (Qiagen, Q33238) and sizes were measured using a Tapestation 4150 (Agilent, G2992AA) and screentape (Agilent, 5067-5562). Libraries were then normalized, pooled, and sequenced using Illumina Nextseq 2000 (Illumina, 20038897) at 750 μM loading density using a Nextseq 2000, P3 reagents 200 cycle flowcell (Illumina, 20040560). Sequencing run was setup according to Illumina Stranded mRNA prep ligation suggested read 1 (76), index 1 (10), index 2 (10), read 2 (76). The modulation of Myctarget gene expression upon treatment with MRT-2359 was analyzed in RNAseq profiles of both Myc-driven (NCI-Hi 155) and non-Mycdriven (NCI-H2023) cell lines. Using a set of annotated Myctarget genes (MYC_TARGETS_V1) from the Broad molecular signatures database [Liberzon, et al. (2015, Cell Systems)], an aggregate score was computed for each RNASeq profile using a single-sample gene-set projection method (ssGSEA, [Barbie et. al., 2009]) via the gsva package in R. Given a single profile, the method first rank-orders the genes in the profile according to their absolute expression (log 2(TPM+1)) values and then computes an enrichment score by comparing the rank distributions of genes in the Myc targets gene-set to the remaining genes. The raw scores were normalized by computing z-scores relative to vehicle treated DMSO samples independently for each cell line tested.

In vivo efficacy study: NCI-H1155, MM1 S, WSU-DLCL2, DOHH-2 cells were maintained in vitro in individual medium and conditions as recommended by the providers and incubated at 37° C. in an atmosphere of 5% CO2 in air. 0.5 million (NCI-H1155), 10 million (MM1S) or 5 million (WSU-DLCL2 and DOHH-2) cells were resuspended in typically 0.1-0.2 mL of PBS with Matrigel (50:50) and were inoculated into 6-8 weeks old BALB/c nude female (NCI-H115, WSU-DLCL2), NOD/SCID (MM1S) or CB-17SCID (DOHH-2) mice and allowed to grow to 150-200 mm3 in size. Mice were dosed orally (PO) and daily (QD) with either vehicle or Compound 345 typically at 1.0 mg/kg, 3.0 mg/kg and 10 mg/kg. Compound 345 was formulated in 5% DMSO/95% (30% w/v HP-β-CD in water) and prepared freshly prior to dosing as a clear solution. Solutions were used within 2 hr from preparation. Lenalidomide PO dosed at 50 mg/kg was used as positive control for the MM1S model. CHOP was used as positive control for the DOHH-2 and WSU-DLCL2 models. CHOP was composed of cyclophosphamide (30 mg/kg, i.p.), doxorubicin (2.47 mg/kg, i.v.), vincristine (0.375 mg/kg, i.v.) all dosed at Day 1 and Day 2 as well as prednisone (0.15 mg/kg, PO) dosed from day 1 to 5 and day 22 to 26. Tumor volumes were measured at least twice weekly during the treatment period using a caliper and the volume was expressed in mm3 using the formula: V=0.5 a×b2 where a and b were the long and short diameters of the tumor, respectively. The tumor volume was then used tumor growth inhibition TGI (%) values calculations. TGI (%) was calculated according to the following equation: TGI (%)=(1−(TVTreatment/Dn−TVTreatment/D1)/(TVControl/Dn−TVControl/D1))×100%. Data are expressed as the mean±SEM. All data was analyzed using GraphPad Prism 8.1.2 (GraphPad).

GSPT1 and N-Myc western blotting analyses from NCI-H1155 tumors: The NCI-H1155 model was run as described previously and tumors were harvested following 5 consecutive daily PO doses of Compound 345 at 1.0 and 10 mg/kg. Tumors were lysed in 1× lysis buffer (CST, Cat. No. 9803, Lot No. 0000122383) supplemented with protease (Sigma, Cat. No. P8340) and phosphatase ((Sigma, Cat. No. P5726 and P0044) cocktail inhibitors using TissueLyser II (QIAGEN #85300). Samples were spined at 15,000 rpm in an Eppendorf microfuge for 15 min at 4° C. The protein supernatant was collected in a new 1.5 ml tube and the protein concentrations were determined by the Pierce BCA kit (Thermo, Cat. No. 23225). The tumor lysate samples were mixed with 5×SDS-PAGE loading buffer boiled for 5 min and brought to ice for 5 min. 30 μg of protein was loaded onto a NUPAGE 4-12% BT gel (Invitrogen, Cat. No. NPO336BOX) for separation. Gels were run in SDS-page running buffer and with a constant voltage (100V) for 2 hr. Gels were transferred onto a 0.2 μm nitrocellulose membrane using the IBLOT transfer stacks (Invitrogen). Membranes were blocked in blocking buffer (Li-COR, Cat. No. 927-60001) for at least 1 hr and incubated overnight at 4° C. with either of the following primary antibody: anti-GSPT1 diluted 1/1000 (Sigma, HPA052488), anti-N-Myc clone D1V2A diluted 1/1000th (Cell Signaling Technology, 84406) or anti-actin (CST, Cat. No. 3700). The next day, a secondary donkey anti-rabbit antibody 1/10000 (Li-COR, Cat. No. 926-32213) was used incubating at RT for at least 45 min. Membranes were imaged on the Odyssey imager (Li-COR) using fluorescence or cheminulinescence detection. Data visualization was performed in GraphPad Prism 8.1.2 (GraphPad) representing Compound 345 levels in plasma on they-axis (in ng/mL) and GSPT1 and N-Myc total protein levels on the secondary y-axis (% relative to vehicle). Data are expressed as mean±SEM derived from at least three different mice and collected at 0, 2, 6, 12, 24, 48, 72 and 96 hr as indicated.

Anti-tumor activity in L- and N-Myc positive NSCLC PDXs: To assess the anti-tumor activity of Compound 345 in vivo and its association with N-Myc and L-Myc expression, we selected a panel of non-small cell lung cancer patient-derived xenografts (PDXs; N=14 with high N-Myc or L-Myc mRNA expression and N=19 with low N-Myc and low L-Myc mRNA expression). mRNA expression levels for the PDXs were measured using RNAseq and quantified in FPKM units. We used log 2FPKM cutoff of 1.5 and 3.4 for N-Myc and L-Myc expression respectively. PDX tumor fragments (2-3 mm in diameter) were inoculated subcutaneously at the upper right dorsal flank of 6-8 weeks old female BALB7c or NODS/CID nude mice. Treatment with Compound 345 was initiated when the tumors reached 80-120 mm3 in size (n=3 mice/arm). Mice were PO dosed with vehicle or Compound 345 at 10 mg/kg daily and tumor volumes recorded at least twice a week. To assess the activity of Compound 345 in N/L-Myc-high tumors we ran a Kaplan-Meier survival analysis. For each mouse, we calculated the first day the tumor volume reached 800 mm3 as the endpoint. If for a mouse, the tumor volume never reached 800 mm3, that case was annotated as a censored case and the last measurement day was used as the event time. Kaplan-Meier curves were generated in R (ver 4.0.2, survminer package) and log-rank (Mantel-Cox) test P-values were calculated using R (ver 4.0.2, survival package) to assess the differences in survival curves between the Compound 345-treated and vehicle treated samples. Similar analysis was done in N/L-Myc-low tumors separately.

1. A method of treating a patient suffering from a Myc-driven tumor, comprising:
(a) determining the expression level of one or more Myc transcription factor biomarkers in a biological sample obtained from the patient; and
(b) treating the patient with a treatment regimen comprising administering a therapeutically effective amount of a GSPT 1 negative modulator if the expression level of the one more Myc transcription factor biomarkers is greater than a reference level for the one more Myc transcription factor biomarkers.

2. The method of claim 1, wherein the biological sample comprises tumor cells or tumor nucleic acid.

3. The method of claim 1, wherein the step of determining comprises acquiring data.

4. The method of claim 1, wherein the step of determining comprises obtaining a biological sample and measuring expression or having a biological sample obtained and having expression measured.

5. The method of claim 1, wherein the tumor nucleic acid is tumor DNA or tumor RNA.

6. The method of claim 1, wherein the step of determining expression level comprising measuring the copy number a gene encoding a Myc transcription factor biomarker.

7. The method of claim 1, wherein the one or more Myc transcription factor biomarkers are selected from the group consisting of: L-Myc, N-Myc, c-Myc, EIF4EBP1 and EIF4EBP2.

8. The method of claim 1, wherein the method further comprises treating the patient with a treatment regime other than administering a GSPT1 negative modulator if the expression level of the one more Myc transcription factor biomarkers is not greater than a reference level for the one more Myc transcription factor biomarkers.

9. The method of claim 1, wherein the GSPT1 negative modulator is a targeted protein degraders that promotes degradation of GSPT1 protein in cells.

10. The method of claim 1, wherein the GSPT1 negative modulator is a molecular glue degrader.

11. The method of claim 1, wherein the biological sample is obtained before the patient is treated with a GSPT1 negative modulator.

12. The method of claim 1, wherein the biological sample is obtained after the patient is treated with a GSPT1 negative modulator.

13. A method of treating a patient suffering from a Myc-driven tumor, comprising:
(a) determining the phosphorylation level of one or more of EIF4EBP1 and EIF4EBP2 in a biological sample obtained from the patient; and
(b) treating the patient with a treatment regimen comprising administering a therapeutically effective amount of a GSPT1 negative modulator if the phosphorylation level of one or more of EIF4EBP1 and EIF4EBP2 is greater than a reference level for the one more Myc transcription factor biomarkers.

14. The method of claim 13, wherein the step of determining comprises acquiring data.

15. The method of claim 13, wherein the step of determining comprises obtaining a biological sample and measuring expression or having a biological sample obtained and having expression measured.

16. The method of claim 13, wherein the method further comprises treating the patient with a treatment regime other than administering a GSPT1 negative modulator if the expression level of the one more Myc transcription factor biomarkers is not greater than a reference level for the one more Myc transcription factor biomarkers.

17. The method of claim 13, wherein the GSPT1 negative modulator is a targeted protein degraders that promotes degradation of GSPT1 protein in cells.

18. The method of claim 13, wherein the GSPT1 negative modulator is a molecular glue degrader.

19. The method of claim 13, wherein the biological sample is obtained before the patient is treated with a GSPT 1 negative modulator.

20. The method of claim 13, wherein the biological sample is obtained after the patient is treated with a GSPT1 negative modulator.

21. A method of treating a patient suffering from a Myc-driven tumor, comprising:
(a) identifying a patient having a Myc-driver tumor; and
(b) treating the patient with a treatment regimen comprising administering a therapeutically effective amount of a GSPT 1 negative modulator.

22. A method of treating a patient suffering from a Myc-driven tumor, comprising administering a therapeutically effective amount of a GSPT1 negative modulator.

23. The method of claim 21, wherein the GSPT1 negative modulator is a targeted protein degraders that promotes degradation of GSPT 1 protein in cells.

24. The method of claim 23, wherein the GSPT1 negative modulator is a molecular glue degrader.

25. A method for determining a treatment for a patient suffering from a Myc-driven tumor from whom a biological sample was obtained and the expression level of one or more Myc transcription factor biomarkers was measured, the method comprising selecting a treatment regimen from the group consisting of:
(a) treating with a therapeutically effective amount of a GSPT1 negative modulator if the expression level of the one more Myc transcription factor biomarkers is greater than a reference level for the one more Myc transcription factor biomarkers; and
(b) treating with a therapeutic agent that other than a GSPT1 negative modulator if the expression level of the one more Myc transcription factor biomarkers is not greater than a reference level for the one more Myc transcription factor biomarkers.

26. The method of claim 1, wherein the Myc-driven cancer is selected from the group consisting of: breast cancer, small cell lung carcinoma, non-small cell lung carcinoma, a neuroendocrine cancer, acute myelogenous leukemia, lymphoma, and multiple myeloma.

27. The method of claim 1, wherein the GSPT1 negative modulator is a compound or a pharmaceutically acceptable salt or stereoisomer thereof of formula I: 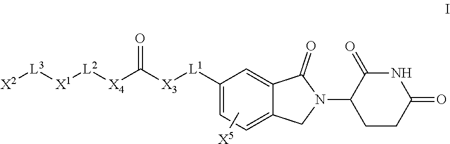
wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ forms together with X⁴ a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₆ alkylhydroxy;
X³ is —NH—, —O—;
X⁴ is —NH—, —CH₂—;
X⁵ is H, linear or branched C₁₋₆ alkyl, —C₁₋₄ alkoxy, —CN, halogen, CF₃, CHF₂, CMeF₂, OCF₃, OCHF₂;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

28. The method of claim 1, wherein the GSPT1 modulator is a compound or a pharmaceutically acceptable salt or stereoisomer thereof of formula II, 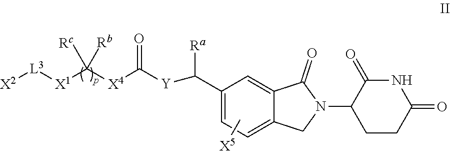
wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alky, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, C₆₋₁₀ aryloxy, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X⁴ is —NH—;
X⁵ is H, linear or branched C₁₋₆ alkyl, —C₁₋₄ alkoxy, —CN, halogen, CF₃, CHF₂, CMeF₂, OCF₃, OCHF₂;
Y is N or O;
R^{a} is a H or C₁₋₄ alkyl;
R^{b}, R^{c} are independently of each other H, C₁₋₄ alkyl, preferably methyl, ethyl, or halogen, preferably F;
L³ is a covalent bond, —O—, —C₁₋₄ alkoxy or C₁₋₆ alkyl, which is unsubstituted or substituted with one or more of C₁₋₄ alkyl, halogen; and
p is 0, 1, 2.

29. The method of claim 1, wherein the compound of formula 1 is a compound or a pharmaceutically acceptable salt or stereoisomer thereof of formula Va: 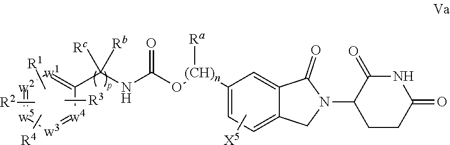
wherein
w¹, w², w³, w⁴, w⁵ are independently of each other selected from C and N, with the proviso that at least three of w¹, w², w³, w⁴, w⁵ are C;
X⁵ is H, linear or branched C₁₋₆ alkyl, —C₁₋₄ alkoxy, —CN, halogen, CF₃, CHF₂, CMeF₂, OCF₃, OCHF₂;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl, R^{b}, R^{c} are independently of each other H, linear or branched C₁₋₄ alkyl; n is 1, or 2; and p is 0 or 1.

30. The method of claim 1, wherein the GSPT 1 negative modulator is selected from any of Compounds 1-160, 201-440 and 501 to 573 and pharmaceutically acceptable salts thereof.

31. The method of claim 1, wherein the GSPT1 negative modulator is selected from any of: 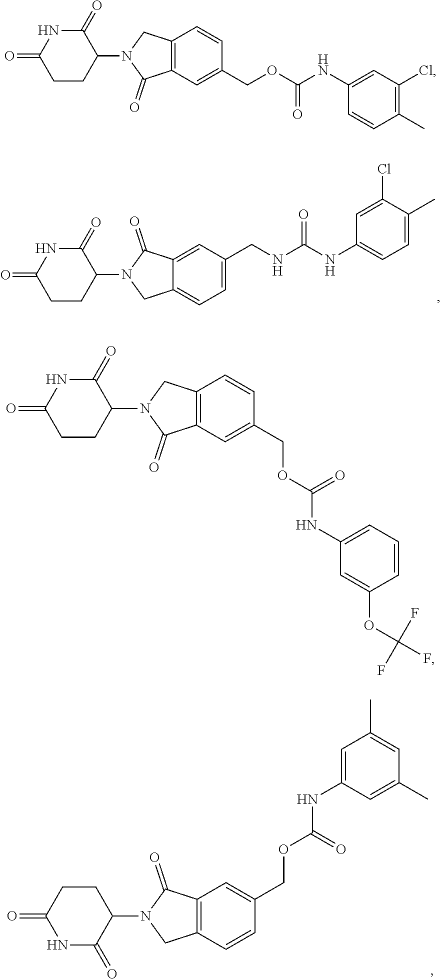 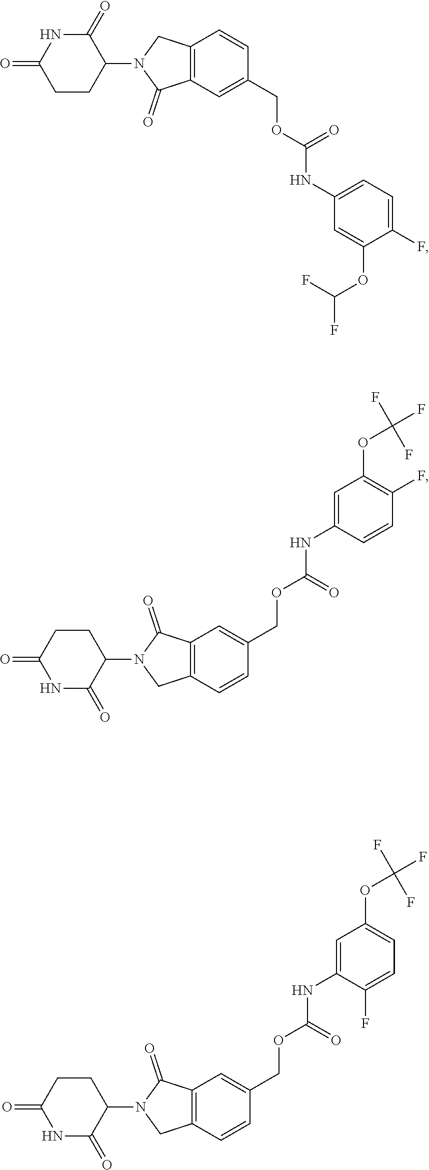 [SMILES: O=C1CCC(N2Cc3ccc(COC(=O)Nc4ccc(F)c(OC(F)F)c4)cc3C2=O)C(=O)N1 ; O=C1CCC(N2Cc3ccc(COC(=O)Nc4ccc(F)c(OC(F)(F)F)c4)cc3C2=O)C(=O)N1 ; O=C1CCC(N2Cc3ccc(COC(=O)Nc4cc(OC(F)(F)F)ccc4F)cc3C2=O)C(=O)N1]
and pharmaceutically acceptable salts thereof.
